(19) United States
(12) Patent Application Publication
(10) Pub. No.: US 2026/0193668 A1
(43) Pub. Date: Jul. 9, 2026

(54) NOVEL PEPTIDES AND USE THEREOF FOR MODULATING PROTEIN ACCUMULATION

(71) Applicants: UNIVERSITE TOULOUSE III - PAUL SABATIER, TOULOUSE Cedex 9 (FR); CENTRE NATIONAL DE LA RECHERCHE SCIENTIFIQUE, PARIS (FR); MICROPEP TECHNOLOGIES, AUZEVILLE TOLOSANE (FR)

(72) Inventor: Jean-Philippe COMBIER, CASTANET-TOLOSAN (FR)

(73) Assignees: UNIVERSITE TOULOUSE III - PAUL SABATIER, TOULOUSE Cedex 9 (FR); CENTRE NATIONAL DE LA RECHERCHE SCIENTIFIQUE, PARIS (FR); MICROPEP TECHNOLOGIES, AUZEVILLE TOLOSANE (FR)

(21) Appl. No.: 19/121,841

(22) PCT Filed: Oct. 19, 2023
(86) PCT No.: PCT/EP2023/079154

§ 371 Date: Apr. 17, 2025

(30) Foreign Application Priority Data
Oct. 19, 2022 (FR) FR2210830

(51) Int. Cl. C12N 15/82; A01N 37/46; A01P 13/00; A01P 21/00

(52) U.S. Cl. CPC C12N 15/8271; A01N 37/46; A01P 13/00; A01P 21/00; C12N 15/8262

(57) ABSTRACT

Novel non-naturally occurring peptides that are not naturally produced by a plant (cPEPs) and novel naturally occurring peptides that are naturally produced by the plant (altPEPs). Also, the method for preparing the cPEPS and altPEPs and the use of the cPEPS and altPEPs for modulating the accumulation of specific proteins in the plant.

### FIELD OF THE INVENTION

The present invention relates to novel peptides (cPEPs & altPEPs), to a method for the preparation thereof, and to the use thereof for modulating the accumulation of specific proteins.

### PRIOR ART

Generally, peptides are short sequences of 2 to around 100 amino acids. They are often highly active molecules, such as hormones or venom compounds.

In plants, peptides fulfil numerous biological functions, such as development or defence mechanisms. As peptide detection is relatively difficult, only a limited number of peptides have been identified, probably underestimating the quantity and role of peptides in these organisms.

Most of the peptides characterised in plants probably result from the degradation of functional proteins. However, it has been shown that primary transcripts of plant microRNAs (miRs) actually contain small open reading frames (miORFs) encoding regulatory peptides, called miPEPs (Lauresserg ues D et al. Primary transcripts of microRNAs encode regulatory peptides. Nature. 2015 Apr. 2; 520(7545):90-3.). The miPEPs are produced at the same site as the miRs from which they originate and enhance transcription of the corresponding pri-miRs. The activity of a miPEP is highly specific to the corresponding miR, making it possible to up-regulate the chosen miR without affecting the expression of other miRs. The use of miPEPs can modulate the expression of a gene if it is regulated by a miR, itself regulated by a miPEP (WO 2015/063431).

### BRIEF OVERVIEW

In this context, the following disclosure provides a universal, and readily exploitable, means for specifically modulating the accumulation of a selected protein in a plant using either a non-naturally occurring peptide (Invention No. 1; cPEP), i.e. a peptide that is not naturally produced by the plant, or a naturally occurring peptide (Invention No. 2; altPEP), i.e. a peptide that is naturally produced by the plant.

One aspect of these inventions is to propose a method for preparing and determining a “cPEP” or “altPEP” peptide capable of modulating the accumulation (expression) of a specific protein in a plant cell. A second aspect of these inventions is to propose a method for modulating the accumulation of a protein in a plant using a cPEP or an altPEP. A third aspect of these inventions is to propose the use of a cPEP or an altPEP to modulate the accumulation of a protein in a plant. A fourth aspect of these inventions is to propose a method for promoting, slowing down or preventing the development of a plant. A fifth aspect of these inventions is to propose cPEP peptides or altPEP peptides to modulate the accumulation of a protein in a plant. Other complementary aspects of these inventions concern a nucleic acid encoding a cPEP or an altPEP, compositions comprising a cPEP or an altPEP and modified or transgenic plants comprising a cPEP or an altPEP.

### DETAILED DESCRIPTION

Considering the first invention (cPEP), a first aspect thereof concerns a method for preparing and determining a cPEP, said cPEP:
having a size from 4 to 70 amino acids, in particular from 4 to 41 amino acids;
being capable of modulating the accumulation of a protein in a plant cell; and
not being capable of modulating the accumulation of the mRNA encoding said protein,
   said method comprising:
a. a step for determining the nucleic acid sequence of the messenger RNA (mRNA) encoding said protein;
b. a step for determining within this mRNA the nucleic acid sequence naturally translated in said plant cell and which corresponds to the open reading frame encoding said protein;
c. a step for determining a fragment of this nucleic acid sequence naturally translated in the said plant cell, said fragment having a size of 3n nucleotides, n being from 4 to 70, in particular n being from 4 to 41, and said fragment having a size smaller than that of the nucleic acid sequence naturally translated in said plant cell;
d. a step for producing the peptide encoded by said fragment; and
e. a comparison step:
   between the accumulation of said protein in a plant cell in the presence of said peptide and the accumulation of said protein in a plant cell of the same type in the absence of said peptide; and/or
   between the phenotype of a plant in the presence of said peptide and the phenotype of a plant of the same type in the absence of said peptide,
wherein:
   a difference in the amount of said protein in the presence of said peptide compared to the amount of said protein in the absence of said peptide; and/or
   a difference in the phenotype in the presence of said peptide compared with the phenotype in the absence of said peptide,
indicates that said peptide is a cPEP capable of modulating the accumulation of said protein in a plant cell.

The present invention is based on the Inventors' unexpected observation that it is possible to specifically modulate the accumulation of a protein using a particular peptide not produced naturally, the sequence of which corresponds to the (artificial) translation of a fragment of the messenger RNA (mRNA) encoding said protein, said fragment being chosen from the nucleic acid sequence naturally translated in said plant cell and which corresponds to the open reading frame encoding said protein.

In this invention, the term “cPEP” (complementary peptide) refers to an artificial peptide capable of specifically modulating the accumulation of a protein when introduced into a plant cell.

According to the invention, a cPEP is not naturally present in a plant cell. This means that the plant cell contains the cPEP information but does not contain the nucleic sequence capable of enabling its expression. Only the peptide sequence of the cPEP can be deduced from the sequence of the mRNA encoding the protein whose accumulation is to be modulated.

A cPEP is only present in a plant cell once it has been introduced in the form of a peptide or in the form of a nucleic acid encoding said peptide.

The specificity of the cPEP with respect to a target protein (a target gene) is determined by its amino acid sequence. The sequence of a cPEP corresponds to the in silico (artificial) translation of a fragment of the mRNA encoding said protein, said fragment being chosen from the nucleic acid sequence naturally translated in said plant cell and which corresponds to the open reading frame encoding said protein.

The peptide sequence of a cPEP can therefore be determined from a fragment of the mRNA encoding said protein by applying, from the first nucleotide of said fragment, the genetic code assigning a specific amino acid to each triplet of nucleotides (AUC=Isoleucine, ACA=Threonine, etc.).

In the invention, the mRNA fragment used to determine the cPEP sequence can be selected from any of the three existing reading frames on the mRNA sequence. In other words, a fragment can be selected from reading frames +1, +2 or +3. In this respect, it is possible that all three reading frames contain the information of a cPEP, just as it is possible that only one of the three reading frames (the +1, the +2 or the +3) or two of the three reading frames (the +1 and the +2, the +1 and the +3, or the +2 and the +3) contain the information of a cPEP.

In the invention, the term “reading frame” refers to the grouping of nucleotides making up a nucleic acid sequence into consecutive triplets (or codons), which follow one another without interruption or overlap.

Generally speaking, cPEPs have a size from 4 to 70 amino acids, in particular a size from 4 to 41 amino acids, in particular a size from 5 to 40 amino acids, from 7 to 20 amino acids or more particularly a size from 8 to 15 amino acids. Consequently, the sequence of a cPEP corresponds to the translation of a fragment comprising from 4 to 70 triplets of nucleotides, in particular a fragment comprising from 4 to 41 triplets of nucleotides, in particular a fragment comprising from 5 to 40 triplets of nucleotides, from 7 to 20 triplets of nucleotides or more particularly a fragment comprising from 8 to 15 triplets of nucleotides.

In other words, the sequence of a cPEP corresponds to the translation into amino acids of a fragment of “3n” nucleotides of the mRNA of the target protein, n being from 4 to 70, in particular from 4 to 41, in particular from 5 to 40, from 7 to 20 or more particularly from 8 to 15.

For example, if n is equal to 5, the cPEP has a size of 5 amino acids and corresponds to the translation of a fragment of 15 (=3×5) nucleotides. For example, if n is equal to 40, the cPEP is 40 amino acids in size and corresponds to the translation of a fragment of 120 (=3×40) nucleotides. For example, if n is equal to 70, the cPEP is 70 amino acids in size and corresponds to the translation of a fragment of 210 (=3×70) nucleotides. And so on.

The cPEPs have a size from 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids, and correspond respectively to the translation of fragments of 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, 204, 207 or 210 nucleotides.

In one embodiment, the invention relates to the method for preparing and determining a cPEP as described above, wherein said fragment has a size of 3n nucleotides, n being comprised:
from 4 to 41;
from 5 to 40;
from 7 to 20; or
from 8 to 15.

In one embodiment, the invention relates to the method for preparing and determining a cPEP as described above, wherein said peptide has a size selected from: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 and 70 amino acids.

In one embodiment, the invention relates to the method for preparing and determining a cPEP as described above, wherein said cPEP has a size smaller than that of said protein (i.e. the protein whose accumulation is modulated by the cPEP).

As indicated above, cPEPs have the ability to specifically modulate the accumulation of a protein without affecting the accumulation of the corresponding mRNA. In other words, the addition of a cPEP to a plant cell does not modify the quantity of mRNA used to express the protein that it has the capacity to regulate, but only the quantity of the said protein.

According to the invention, the term “protein” refers to a sequence of amino acids whose information is encoded by a gene present in the genome of a plant cell. By “gene” is therefore meant, in particular, the nucleic acid sequence necessary for the synthesis of the said protein. A gene also includes more than the nucleotides encoding the amino acid sequence of the protein. For example, a gene includes the DNA sequences required to synthesise a pre-messenger (pre-mRNA), which is then matured by the cellular machinery into a messenger RNA (mRNA). This can then be translated into a protein via the ribosomes.

In view of the above, it is clear that pre-messenger RNA (pre-mRNA) has not undergone splicing and is likely to contain introns, whereas mature messenger RNA (mRNA) may have undergone splicing and contains only exons.

To prepare a cPEP capable of modulating the accumulation of a protein, it is necessary to translate a fragment of the mRNA encoding said protein, said fragment being chosen from the nucleic acid sequence naturally translated in said plant cell and which corresponds to the open reading frame encoding said protein, which comprises neither the 5′-UTR region nor the 3′-UTR region of the mRNA.

In the invention, “modulation” of the accumulation of a protein designates either an increase in the accumulation of said protein (i.e. an increase in the quantity of protein in the plant cell), or a decrease in the accumulation of said protein (i.e. a decrease in the quantity of protein in the plant cell). In other words, one embodiment of the invention relates to the method for preparing and determining a cPEP as described above, wherein said modulation of the accumulation of said protein induced by said cPEP is:
a reduction in the accumulation of the said protein; or
an increase in the accumulation of the said protein.

The increase and decrease in the accumulation of said protein can be measured and monitored using methods well known to those skilled in the art, such as coupling the protein to a marker using specific expression cassettes, or a Western blot.

In one embodiment, the invention relates to the method for preparing and determining a cPEP as described above, wherein in step e., the amount of protein in the presence of said peptide is greater than the amount of protein in the absence of said peptide. In other words, in the presence of a cPEP promoting increased protein accumulation, translation of the corresponding mRNA is increased, leading to greater production of the protein without altering the amount of said mRNA.

In another embodiment, the invention relates to the method for preparing and determining a cPEP as described above, wherein in step e., the amount of protein in the presence of said peptide is less than the amount of protein in the absence of said peptide. In other words, in the presence of a cPEP favouring a reduction in the accumulation of the protein, the translation of the corresponding mRNA is reduced (inhibited), which leads to a lower production of the protein without the quantity of said mRNA being modified.

In the invention, although the fragment of the mRNA encoding said cPEP is located within the nucleic acid sequence naturally translated in said plant cell and which corresponds to the open reading frame encoding said protein, said fragment as such is not naturally translated in said plant cell. This is the case regardless of the reading frame used. The existence of a cPEP within the said plant cell is therefore artificial and originates from human action. To achieve this, it is possible either to artificially introduce said cPEP as such, or to introduce an expression cassette comprising the nucleic acid sequence encoding said cPEP and the means of expressing it in said plant cell.

In the invention, the nucleic acid sequence naturally translated in the said plant cell, which comprises a fragment carrying the information of a cPEP, is a region of the mRNA described as “coding”, i.e. it corresponds to a region of the mRNA which codes for all or part of the functional protein. This sequence therefore corresponds to the main open reading frame, which codes for the protein whose accumulation is to be modulated.

In the invention, the terms “open reading frame” and “ORF” (open reading frame) are equivalent and can be used interchangeably. They correspond to a sequence of nucleotides (nucleic acids) in a DNA or RNA molecule that can potentially encode a peptide or protein: the said open reading frame begins with a START codon (the START codon generally encoding a methionine), followed by a series of codons (each codon encoding an amino acid), and ends with a STOP codon (the STOP codon not being translated).

The mRNA coding region therefore corresponds to the genetic sequence delimited by the START codon or initiation codon (most often encoding a methionine) at the 5′ end and by the STOP codon at the 3′ end. The mRNA coding region therefore does not include any intronic sequences that may be present in the sequence of a gene or pre-messenger RNA (pre-mRNA), or the 5′UTR and 3′UTR regions, as these are not translated and therefore do not code for part of the gene's functional protein.

In the invention, the sequence of a cPEP is determined by (artificially) translating a fragment of the mRNA of the protein whose accumulation is to be modulated, said fragment being chosen from the (coding) nucleic acid sequence naturally translated in said plant cell and which corresponds to the open reading frame coding for said protein. Also, the same nucleic acid sequence naturally translated in said plant cell can give different cPEPs depending on the mRNA fragment chosen. Furthermore, this same mRNA fragment may also give different cPEPs depending on the reading frame used to translate it (artificially), i.e. depending on the grouping of the nucleotides of the sequence into consecutive triplets. As mentioned above, a translation can be carried out in three different reading frames, potentially leading to three different cPEPs.

According to the invention, the “+1” reading frame corresponds to the reading frame determined by the initiation codon of the protein, i.e. the START codon of the open reading frame naturally used for mRNA translation. In other words, in the case of an mRNA fragment corresponding to a coding region and translated according to the +1 reading frame, the cPEP obtained has a sequence identical to that of a fragment of the amino acid sequence of the protein naturally encoded by said mRNA.

The “+2” and “+3” reading frames correspond to reading frames that are not (or are only rarely) used naturally for mRNA translation. According to the invention, the “+2” reading frame corresponds to the reading frame shifted by one nucleotide at 3′ (or by two nucleotides at 5′) relative to the open reading frame naturally used for translation of the mRNA and the protein it encodes. According to the invention, the “+3” reading frame corresponds to the reading frame shifted by two nucleotides at 3′ (or one nucleotide at 5′) with respect to the open reading frame naturally used for translation of the mRNA and the protein it encodes.

Generally, in the case of an mRNA fragment corresponding to a coding region and translated according to the +2 or +3 reading frame, the cPEPs obtained have a different sequence to that of a fragment of the amino acid sequence of the protein naturally encoded by the said mRNA.

In one embodiment, the invention relates to the method for preparing and determining a cPEP as described above, wherein said fragment is devoid of:
the initiation codon AUG encoding an initiator methionine; or
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

In one embodiment, the invention relates to the method for preparing and determining a cPEP as described above, wherein said fragment is devoid of:
the AUG initiation codon encoding a initiator methionine; and
a STOP codon chosen from: UAG, UGA and UAA,
   and wherein said fragment is selected from:
either in the same reading frame as the open reading frame encoding said protein;
or in a reading frame shifted by one or two nucleotides relative to the open reading frame encoding said protein.

It is therefore understood that a cPEP, according to the above embodiments, comprises either an AUG codon (and no STOP codon), or a STOP codon (and no AUG codon), or neither of these two elements. In the present case, it is up to the person skilled in the art to add, if necessary, the missing element or elements to enable, in a non-limiting manner, either the in vitro production of a cPEP by means, for example, of a microorganism and then its use (in a composition, for example), or the introduction of its sequence and the means of expressing it via a vector into a plant cell or a plant.

In one embodiment, the invention therefore relates to the method for preparing and determining a cPEP as described above, wherein said fragment comprises an AUG initiation codon encoding an initiator methionine and lacks a STOP codon chosen from the codons: UAG, UGA and UAA. The invention also relates to the method for preparing and determining a cPEP as described above, wherein said fragment is devoid of an initiation codon AUG encoding an initiator methionine and comprises a STOP codon selected from codons: UAG, UGA and UAA.

In view of the foregoing, it is understood that in another embodiment, the invention relates to the method for preparing and determining of a cPEP as described above, said cPEP:
having a size from 4 to 70 amino acids, in particular from 4 to 41 amino acids;
being capable of modulating the accumulation of a protein in a plant cell; and
not being capable of modulating the accumulation of the mRNA encoding said protein,
   said method comprising:
a. a step for determining the nucleic acid sequence of the messenger RNA (mRNA) encoding said protein;
b. a step for determining within this mRNA the nucleic acid sequence naturally translated in said plant cell and which corresponds to the open reading frame encoding said protein;
c. a step for determining, within this nucleic acid sequence naturally translated in said plant cell, a non-naturally translated fragment thereof, said fragment not naturally translated:
   having a size of 3n nucleotides, n being from 4 to 70, in particular n being from 4 to 41,
   having a size smaller than that of the nucleic acid sequence naturally translated in the said plant cell,
   lacking the initiation codon AUG encoding an initiator methionine and/or a STOP codon chosen from the codons: UAG, UGA and UAA, and
   being chosen either in the same reading frame as the open reading frame encoding said protein, or in a reading frame shifted by one or two nucleotides relative to the open reading frame encoding said protein;
d. a step for producing the peptide encoded by said non-naturally translated fragment; and
e. a comparison stage:
   between the accumulation of said protein in a plant cell in the presence of said peptide and the accumulation of said protein in a plant cell of the same type in the absence of said peptide; and/or
   between the phenotype of a plant in the presence of said peptide and the phenotype of a plant of the same type in the absence of said peptide,
wherein:
   a difference in the amount of said protein in the presence of said peptide compared to the amount of said protein in the absence of said peptide; and/or
   a difference in the phenotype in the presence of said peptide compared with the phenotype in the absence of said peptide,
indicates that said peptide is a cPEP capable of modulating the accumulation of said protein in a plant cell.

In one embodiment, the invention relates to the method for preparing and determining a cPEP as described above, wherein the translation of the mRNA fragment is carried out in the same reading frame as the open reading frame of the nucleic acid sequence naturally translated in the said plant cell. In other words, the invention relates to the method for preparing and determining a cPEP as described above, wherein the translation of the mRNA fragment is carried out in the reading frame determined by the initiation codon of the open reading frame of the said protein.

In one embodiment, the invention relates to the method for preparing and determining a cPEP as described above, wherein the translation of the mRNA fragment is carried out in a reading frame different from the open reading frame of the nucleic acid sequence naturally translated in the said plant cell. In particular, the invention relates to the method for preparing and determining a cPEP as described above, wherein the translation of the mRNA fragment is carried out in a reading frame shifted by one nucleotide 3′ (or by two nucleotides 5′) with respect to the open reading frame of the nucleic acid sequence naturally translated in said plant cell. In particular, the invention also relates to the method for preparing and determining a cPEP as described above, wherein the translation of the mRNA fragment is carried out in a reading frame shifted by two nucleotides 3′ (or one nucleotide 5′) with respect to the open reading frame of the nucleic acid sequence naturally translated in the said plant cell. In other words, the invention relates to the method for preparing and determining a cPEP as described above, wherein the translation of the mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in said plant cell.

In one embodiment, the invention relates to the method for preparing and determining a cPEP as described above, wherein said cPEP is a hydrophobic peptide or a hydrophilic peptide. In particular, the invention relates to the method for preparing and determining a cPEP as described above, wherein said cPEP is a hydrophobic peptide. By “hydrophobic peptide” is meant a peptide the amino acid sequence of which comprises more than 50% hydrophobic amino acids. More than 50%” means that the amino acid sequence comprises more than 55%, more than 60%, more than 65%, more than 70%, more than 75% or more than 80% hydrophobic amino acids. By “more than 50%” is also meant that the amino acid sequence comprises at least 51%, at least 56%, at least 61%, at least 66%, at least 71%, at least 76% or at least 81% hydrophobic amino acids. By “hydrophobic amino acids” is meant amino acids selected from: alanine (Ala/A), isoleucine (Ile/I), leucine (Leu/L), methionine (Met/M), phenylalanine (Phe/F), tryptophan (Trp/W), tyrosine (Tyr/Y) and valine (Val/V).

In particular, the invention also relates to the method for preparing and determining a cPEP as described above, wherein said cPEP is a hydrophilic peptide. By “hydrophilic peptide” is meant a peptide the amino acid sequence of which comprises more than 50% hydrophilic amino acids. More than 50%” means that the amino acid sequence comprises more than 55%, more than 60%, more than 65%, more than 70%, more than 75% or more than 80% hydrophilic amino acids. By “more than 50%” is also meant that the amino acid sequence comprises at least 51%, at least 56%, at least 61%, at least 66%, at least 71%, at least 76% or at least 81% hydrophilic amino acids. By “hydrophilic amino acids” is meant amino acids selected from: aspartic acid (Asp/D), glutamic acid (Glu/E), arginine (Arg/R), asparagine (Asn/N), glutamine (Gln/Q), histidine (His/H), lysine (Lys/K), serine (Ser/S) and threonine (Thr/T).

In one embodiment, the invention relates to a method for preparing and determining a cPEP, said cPEP:
having a size from 4 to 70 amino acids, in particular from 4 to 41 amino acids;
being capable of modulating the accumulation of a protein in a plant cell; and
not being capable of modulating the accumulation of the mRNA encoding said protein,
   said method comprising:
a. a step for determining the nucleic acid sequence of the messenger RNA (mRNA) encoding said protein;
b. a step for determining within this mRNA one of the nucleic acid sequences comprising two contiguous parts:
   a part located within a nucleic acid sequence known to be non-coding (i.e. not naturally translated, e.g. 5′UTR or 3′UTR), and
   a part located within a nucleic acid sequence considered to be coding (i.e. naturally translated, e.g. exon);
c. a step for determining within this nucleic acid sequence comprising two contiguous parts of a fragment thereof, said fragment having a size of 3n nucleotides capable of being translated via the genetic code into a peptide, n being from 4 to 70, in particular n being from 4 to 41;
d. a step for producing said peptide; and
e. a comparison step:
   between the accumulation of said protein in a plant cell in the presence of said peptide and the accumulation of said protein in a plant cell of the same type in the absence of said peptide; and/or
   between the phenotype of a plant in the presence of said peptide and the phenotype of a plant of the same type in the absence of said peptide,
wherein:
   a difference in the amount of said protein in the presence of said peptide compared to the amount of said protein in the absence of said peptide; and/or
   a difference in the phenotype in the presence of said peptide compared with the phenotype in the absence of said peptide,
indicates that said peptide is a cPEP capable of modulating the accumulation of said protein in a plant cell.

According to the invention, a cPEP can be produced by any type of means accessible to the person skilled in the art.

A cPEP can be produced either by synthesis or by recombinant expression in homologous or heterologous systems. The cPEP thus produced can then be introduced into a cell to modulate the accumulation of a target protein.

It is also possible to produce a cPEP directly in the plant cell containing the target protein, by artificially introducing a nucleic acid (such as an expression vector) encoding said cPEP.

In one embodiment, the invention relates to the method for preparing and determining a cPEP as described above, wherein, in step d., the said peptide is produced by peptide synthesis or by recombinant expression.

In one embodiment, the invention relates to the method for preparing and determining a cPEP as described above, wherein, in step d., said peptide is produced using a nucleic acid encoding said peptide introduced into a cell.

In one embodiment, the invention relates to the method for preparing and determining a cPEP as described above, wherein, in step e., the production of said peptide is carried out using a nucleic acid encoding said peptide introduced into said plant cell or into said plant.

In one embodiment, the invention relates to a method for preparing and determining a cPEP as described above, wherein, in step e., said peptide is brought into contact with said plant cell or in said plant.

In one embodiment, the invention relates to the method for preparing and determining a cPEP as described above, wherein, in step e., said peptide is present in said plant cell or in said plant following expression of a nucleic acid encoding said peptide in said plant cell or in said plant.

In view of the foregoing, it is understood that another embodiment of the invention relates to the method for preparing and determining a cPEP as described above, wherein, in step e., the presence of said peptide in said plant cell or in said plant results:
the introduction of a nucleic acid sequence encoding said peptide and comprising the means for expressing it; or
the introduction of an amino acid sequence corresponding to the said peptide.

A cPEP can be used to modulate the accumulation of a protein present naturally (i.e. endogenously) or not (i.e. exogenously) in said plant cell or in said plant.

A “protein naturally present in a plant cell or plant” is an endogenous protein encoded by a gene present in the genome of the plant cell or plant without the need for direct or indirect intervention by a human being.

A “protein which is not naturally present in a plant cell or in a plant” corresponds to an exogenous protein encoded by a nucleic acid sequence present on the genome of the plant cell or plant which required the intervention of a human being and the use of means known to the person skilled in the art. Such a nucleic acid sequence may come from the same plant species or from another plant species.

In one embodiment, the invention relates to the method for preparing and determining a cPEP as described above, wherein said protein is of endogenous origin in said plant cells or plants used in step e.

In one embodiment, the invention relates to the method for preparing and determining a cPEP as described above, wherein said protein is of exogenous origin in said plant cells or said plants used in step e., said plant cells or said plants used in step e. then comprising a nucleic acid sequence allowing expression of said protein.

In one embodiment, the invention relates to the method for preparing and determining a cPEP as described above, wherein the accumulation of the said protein is determined using a technique chosen from: Western blot, measurement of enzymatic activity, mass spectrometry and translational fusion. In particular, the invention relates to the method for preparing and determining a cPEP as described above, wherein the accumulation of the said protein is determined using a Western blot.

Surprisingly, the Inventors have found that the use of cPEPs makes it possible to modify the phenotypes of a plant that are visible on a macroscopic scale. It is therefore entirely possible to use cPEPs to confirm (or deny) that the peptide determined in steps a., b. and c., and possibly produced in step d., is a cPEP (or not). This is also made possible by the so-called phenotypic comparison alternative implemented in step e. For example, if the peptide determined on the mRNA of a protein involved in the size of the stem of a plant causes an increase, or a decrease, in the size of the stem of a plant treated with the latter compared with an untreated plant, this means that the said peptide is a cPEP capable of modulating the accumulation of the said protein in the size of the stem.

In the invention, the term “plant” refers generally to:
a set of plant cells organised in all or part of a plant, whatever its stage of development (including the plant in the form of a seed or young shoot);
one or more plant organs (e.g. leaves, roots, stems, flowers);
to one or more plant cells; or
a cluster of plant cells (e.g. a callus).

In the invention, the term “phenotype” designates, in a non-limiting manner, macroscopically visible characteristics such as the number of lateral roots, the number of leaves, the size of the stem, the duration of flowering and resistance to stress. In one embodiment, the invention therefore relates to the method for preparing and determining a cPEP as described above, wherein said protein is involved in at least one plant phenotype chosen from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

According to the invention, a protein is “involved in a phenotype” if a change in its accumulation is associated with a change in the said phenotype. In other words, a protein is involved in a phenotype if it is involved in the character(s) corresponding to the said phenotype.

In view of the foregoing, it is understood that an object of the invention is the method for preparing and determining a cPEP as described above, wherein the phenotype observed in step e. is chosen from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and duration of juvenile phase;
duration of flowering;
resistance to biotic stress;
resistance to abiotic stress; and
the number of cells.

In one embodiment, the invention relates to the method for preparing and determining a cPEP as described above, said cPEP:
having a size from 4 to 70 amino acids, in particular from 4 to 41 amino acids;
being capable of modulating the accumulation of a protein in a plant cell; and
not being capable of modulating the accumulation of the mRNA encoding said protein,
   and wherein the said plant cell (i.e. that wherein it is desired to modulate the accumulation of a protein) belongs to a plant species chosen from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (trefoil), Medicago sativa (alfalfa), Medicago truncatula (lucerne), Nicotiana benthamiana (tobacco), Oryza sativa (rice), Pisum sativum (pea), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In one embodiment, the invention relates to the method for preparing and determining a cPEP as described above, wherein said plant cells or said plants used in step e. belong to: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (trefoil), Medicago sativa (alfalfa), Medicago truncatula (lucerne), Nicotiana benthamiana (tobacco), Oryza sativa (rice), Pisum sativum (pea), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In one embodiment, the invention relates to the method for preparing and determining a cPEP as described above, wherein said plant cell (i.e. the one wherein it is desired to modulate the accumulation of a protein) is an algal cell.

In one embodiment, the invention relates to the method for preparing and determining a cPEP as described above, wherein said plant cells or said plants used in step e belong to an alga.

In one embodiment, the invention relates to the method for preparing and determining a cPEP as described above, wherein said protein is encoded by a gene selected from: Aae15 (Acyl-activating enzyme 15), Aae16 (AMP-dependent synthetase and ligase family protein), Abcg11 (White-brown complex-like protein), Abdcg34 (ABC transporter G family member 34), Acc1 (Acetyl-CoA Carboxylase), Agb1 (GTP binding protein beta 1), Als (Acetolactate synthase (chloroplastic)), Anac076 (NAC domain-containing protein 76), Apg9 (Autophagy 9), Arlb1 (GTP-binding protein 1), Arr1 (Two-component response regulator ARR1), Arr5 (Two-component response regulatorARR5), Arr6 (Two-component response regulatorARR6), At59 (Pectate lyase family protein), Bak1 (Brassinosteroid insensitive 1-associated receptor kinase 1), Bccp1 (Acetyl-CoA Carboxylase (chloroplastic) subunit 1), Bccp2 (Acetyl-CoA Carboxylase (chloroplastic) subunit 2), Bri1 (Brassinosteroid insensitive 1), Bzo2 h3 (bZIP transcription factor family protein), Cesa6 (Cellulose synthase A catalytic subunit 6), Cipk3 (CBL-interacting protein kinase 3), Cks1 (Cyclin-dependent kinases regulatory subunit 1), Cobl8 (COBRA-like protein 8 precursor), Coi1 (Coronatine-insensitive protein 1), Cpk3 (Calcium-dependent protein kinase 3), Crk34 (Cysteine-rich receptor-like protein kinase 34), Cyp705a18 (Cytochrome P450, family 705, subfamily A, polypeptide 18), Cyp71b26 (Cytochrome P450, family 71, subfamily B, polypeptide 26), Cyp78a8 (Cytochrome P450, family 78, subfamily A, polypeptide 8), Cyp97b3 (Cytochrome P450, family 97, subfamily B, polypeptide 3), Dcl1 (Endoribonuclease Dicer homolog 1), Dur3 (Urea-proton symporter DUR3), Ein2 (Ethylene-insensitive protein 2), Emb175 (Pentatricopeptide repeat-containing protein), Emb2726 (Elongation factor Ts family protein), Emb9 (Dihydrofolate synthetase), Epsps (5-enolpyruvylshikimate-3-phosphate (chloroplastic)), Fnr1 (Ferredoxin-NADP[+]-oxidoreductase 1), Fve (Transducin family protein/WD-40 repeat family protein), Ga2ox7 (Gibberellin 2-beta-dioxygenase 7), Gapc (Glyceraldehyde-3-phosphate dehydrogenase), Gcn2 (ABC transporter family protein), Gdi2 (Guanosine nucleotide diphosphate dissociation inhibitor 2), Gln2 (Glutamine synthetase (chloroplastic)), Gsl3 (Callose synthase 2), Hag5 (Histone acetyltransferase of the MYST family 2), Hda18 (Histone deacetylase 18), Hexo1 (Beta-hexosaminidase 1), Hppd (4-hydroxyphenyl-pyruvate-dioxygenase), Hsl1 (B3 domain-containing transcription repressor VAL2), Iaa31 (Indole-3-acetic acid inducible 31), Iqd28 (IQ-domain 28), Jac1 (J-domain protein required for chloroplast accumulation response 1), Jar (Jasmonoyl-L-amino acid synthetase), Kp1 (Kinesin-like protein 1), Lrx2 (Leucine-rich repeat/extensin 2), Mapkkk3 (Mitogen-activated protein kinase kinase kinase 3), Mapkkk5 (Mitogen-activated protein kinase kinase kinase 5), Mfp2 (Multifunctional protein 2), Mrb1 (Transmembrane protein, putative (DUF3537)), Nsp1 (Nodulation signaling pathway 1), Pds (Phytoene desaturase (chloroplastic)), Pen3 (Phosphatidylinositol 3,4,5-trisphosphate 3-phosphatase and protein-tyrosine-phosphatase), Phyb (Phytochrome B), Pif3 (Phytochrome interacting factor 3), Pizza (Brassinosteroid-related acyltransferase 1), Ppox1 (Protoporphyrinogen oxidase (chloroplastic) 1), Ppox2 (Protoporphyrinogen oxidase (chloroplastic) 2), Prp39 (Tetratricopeptide repeat (TPR)-like superfamily protein), PsbA (Photosystem II D1 protein), Pskr1 (Phytosulfokin receptor 1), Rd21 (Granulin repeat cysteine protease family protein), Ring1 (RING/U-box superfamily protein), Ros1 (DNA glycosylase/AP lyase ROS1), Rpt4a (26S proteasome regulatory subunit 10B homolog A), Sfr6 (Mediator of RNA polymerase II transcription subunit 16), Shr (Protein SHORT-ROOT), Shy2 (Auxin-responsive protein IAA3), Skl (EIN2-like protein, nramp transporter), Sps1 (Sucrose phosphate synthase 2F), Spt (Transcription factor SPATULA), Stn8 (Serine/threonine-protein kinase), Tap46 (PP2A regulatory subunit TAP46), Topp6 (Serine/threonine-protein phosphatase PP1 isozyme 7), TubB6 (Tubulin), TubB8 (Tubulin), Uba1a (RNA-binding (RRM/RBD/RNP motifs) family protein), Vim3 (E3 ubiquitin-protein ligase), Sgr1 (Magnesium dechelatase), Abi5 (Abscisic acid (ABA)-insensitive 5), Hsp101 (Heat shock protein 101), Rh10 (ATP-dependent RNA helicase) and Wus (WUSCHEL).

In particular, the invention relates to the method for preparing and determining a cPEP as described above, wherein said protein is encoded by a gene selected from: Cpk3, Dcl1 and Nsp1. In particular, the invention relates to the method for preparing and determining a cPEP as described above, wherein said protein is encoded by the Cpk3 gene. In particular, the invention relates to the method for preparing and determining a cPEP as described above, wherein the said protein is encoded by the Dcl1 gene. In particular, the invention relates to the method for preparing and determining a cPEP as described above, wherein the said protein is encoded by the Nsp1 gene.

The genes Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2 h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexo1, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Ski, Sps1, Spt, Stn8, Tap46, Topp6, TubB6, TubB8, Uba1a, Vim3, Sgr1, Abi5, Hsp101, Rh10 and Wus refer to the proteins indicated in brackets. Of course, the invention also relates to homologous and/or similar genes that may bear different names. For example, in A. thaliana the Gsl3 gene encoding callose synthase 2 is also called Cals2.

In one embodiment, the invention relates to the method for preparing and determining a cPEP as described above, wherein said protein is encoded by an ORF comprising a nucleic acid sequence having at least 80% identity, preferably at least 90% identity, with a sequence chosen from the sequences: SEQ ID NO: 1 (ORF of the Aae15 protein, A. thaliana), SEQ ID NO: 2 (ORF of the Aae16 protein, A. thaliana), SEQ ID NO: 3 (ORF of the abcg11 protein, A. thaliana), SEQ ID NO: 4 (ORF of the Abdcg34 protein, A. thaliana), SEQ ID NO: 5 (ORF of the Acc1 protein, A. thaliana), SEQ ID NO: 6 (ORF of the Agb1 protein, A. thaliana), SEQ ID NO: 7 (ORF of the Als protein, A. thaliana), SEQ ID NO: 8 (ORF of the Anac076 protein, A. thaliana), SEQ ID NO: 9 (ORF of the Apg9 protein, A. thaliana), SEQ ID NO: 10 (ORF of the Arlb1 protein, A. thaliana), SEQ ID NO: 11 (ORF of Arr1 protein, A. thaliana), SEQ ID NO: 12 (ORF of Arr5 protein, A. thaliana), SEQ ID NO: 13 (ORF of Arr6 protein, A. thaliana), SEQ ID NO: 14 (ORF of At59 protein, A. thaliana), SEQ ID NO: 15 (ORF of Bak1 protein, A. thaliana), SEQ ID NO: 16 (ORF of the Bccp1 protein, A. thaliana), SEQ ID NO: 17 (ORF of the Bccp2 protein, A. thaliana), SEQ ID NO: 18 (ORF of the Bri1 protein, A. thaliana), SEQ ID NO: 19 (ORF of the Bzo2 h3 protein, A. thaliana), SEQ ID NO: 20 (ORF of the Cesa6 protein, A. thaliana), SEQ ID NO: 21 (ORF of the Cipk3 protein, A. thaliana), SEQ ID NO: 22 (ORF of the Cks1 protein, A. thaliana), SEQ ID NO: 23 (ORF of the Cobl8 protein, A. thaliana), SEQ ID NO: 24 (ORF of Coi1 protein, A. thaliana), SEQ ID NO: 25 (ORF of Coi1 protein, A. thaliana), SEQ ID NO: 26 (ORF of Cpk3 protein, A. thaliana), SEQ ID NO: 27 (ORF of Cpk3 protein, A. hypochondriacus), SEQ ID NO: 28 (ORF of Cpk3 protein, B. distachyon), SEQ ID NO: 29 (ORF of Cpk3 protein, B. distachyon), SEQ ID NO: 30 (ORF of Cpk3 protein, G. max), SEQ ID NO: 31 (ORF of Cpk3 protein, G. max), SEQ ID NO: 32 (ORF of Cpk3 protein, G. max), SEQ ID NO: 33 (ORF of Cpk3 protein, G. max), SEQ ID NO: 34 (ORF of Cpk3 protein, O. sativa), SEQ ID NO: 35 (ORF of Cpk3 protein, O. sativa), SEQ ID NO: 36 (ORF of Cpk3 protein, S. lycopersicum), SEQ ID NO: 37 (ORF of Cpk3 protein, Z. mays), SEQ ID NO: 38 (ORF of Cpk3 protein, Z. mays), SEQ ID NO: 39 (ORF of Cpk3 protein, Z. mays), SEQ ID NO: 40 (ORF of Cpk3 protein, B. rapa), SEQ ID NO: 41 (ORF of Cpk3 protein, B. rapa), SEQ ID NO: 42 (ORF of Cpk3 protein, H. vulgare), SEQ ID NO: 43 (ORF of Cpk3 protein, H. vulgare), SEQ ID NO: 44 (ORF of Cpk3 protein, S. tuberosum), SEQ ID NO: 45 (ORF of Cpk3 protein, A. palmeri), SEQ ID NO: 46 (ORF of Cpk3 protein, M. truncatula), SEQ ID NO: 47 (ORF of Cpk3 protein, M. truncatula), SEQ ID NO: 48 (ORF of Cpk3 protein, T. aestivum), SEQ ID NO: 49 (ORF of Cpk3 protein, T. aestivum), SEQ ID NO: 50 (ORF of Cpk3 protein, T. aestivum), SEQ ID NO: 51 (ORF of Cpk3 protein, T. aestivum), SEQ ID NO: 52 (ORF of Cpk3 protein, L. perenne), SEQ ID NO: 53 (ORF of Cpk3 protein, L. perenne), SEQ ID NO: 54 (ORF of Cpk3 protein, L. perenne), SEQ ID NO: 55 (ORF of protein Cpk3, L. perenne), SEQ ID NO: 56 (ORF of protein Crk34, A. thaliana), SEQ ID NO: 57 (ORF of protein Cyp705a18, A. thaliana), SEQ ID NO: 58 (ORF of protein Cyp71b26, A. thaliana), SEQ ID NO: 59 (ORF of protein Cyp78a8, A. thaliana), SEQ ID NO: 60 (ORF of Cyp97b3 protein, A. thaliana), SEQ ID NO: 61 (ORF of Dcl1 protein, A. thaliana), SEQ ID NO: 62 (ORF of Dcl1 protein, A. thaliana), SEQ ID NO: 63 (ORF of Dcl1 protein, A. hypochondriacus), SEQ ID NO: 64 (ORF of Dcl1 protein, B. distachyon), SEQ ID NO: 65 (ORF of Dcl1 protein, G. max), SEQ ID NO: 66 (ORF of Dcl1 protein, G. max), SEQ ID NO: 67 (ORF of Dcl1 protein, O. sativa), SEQ ID NO: 68 (ORF of Dcl1 protein, S. lycopersicum), SEQ ID NO: 69 (ORF of Dcl1 protein, Z. mays), SEQ ID NO: 70 (ORF of Dcl1 protein, B. rapa), SEQ ID NO: 71 (ORF of Dcl1 protein, H. vulgare), SEQ ID NO: 72 (ORF of Dcl1 protein, S. tuberosum), SEQ ID NO: 73 (ORF of Dcl1 protein, M. truncatula), SEQ ID NO: 74 (ORF of Dcl1 protein, T. aestivum), SEQ ID NO: 75 (ORF of Dcl1 protein, T. aestivum), SEQ ID NO: 76 (ORF of Dcl1 protein, T. aestivum), SEQ ID NO: 77 (ORF of Dcl1 protein, T. aestivum), SEQ ID NO: 78 (ORF of Dcl1 protein, L. perenne), SEQ ID NO: 79 (ORF of the Dcl1 protein, L. perenne), SEQ ID NO: 80 (ORF of the Dur3 protein, A. thaliana), SEQ ID NO: 81 (ORF of the Ein2 protein, A. thaliana), SEQ ID NO: 82 (ORF of Emb175 protein, A. thaliana), SEQ ID NO: 83 (ORF of Emb2726 protein, A. thaliana), SEQ ID NO: 84 (ORF of Emb9 protein, A. thaliana), SEQ ID NO: 85 (ORF of Epsps protein, A. thaliana), SEQ ID NO: 86 (ORF of Fnr1 protein, A. thaliana), SEQ ID NO: 87 (ORF of the Fve protein, A. thaliana), SEQ ID NO: 88 (ORF of the Ga2ox7 protein, A. thaliana), SEQ ID NO: 89 (ORF of the Gapc protein, N. benthamiana), SEQ ID NO: 90 (ORF of the Gcn2 protein, A. thaliana), SEQ ID NO: 91 (ORF of the Gdi2 protein, A. thaliana), SEQ ID NO: 92 (ORF of Gln2 protein, A. thaliana), SEQ ID NO: 93 (ORF of Gsl3 protein, A. thaliana), SEQ ID NO: 94 (ORF of Hag5 protein, A. thaliana), SEQ ID NO: 95 (ORF of the Hda18 protein, A. thaliana), SEQ ID NO: 96 (ORF of the Hexo1 protein, A. thaliana), SEQ ID NO: 97 (ORF of the Hppd protein, A. thaliana), SEQ ID NO: 98 (ORF of the Hsl1 protein, A. thaliana), SEQ ID NO: 99 (ORF of the Iaa31 protein, A. thaliana), SEQ ID NO: 100 (ORF of the Iqd28 protein, A. thaliana), SEQ ID NO: 101 (ORF of the Jac1 protein, A. thaliana), SEQ ID NO: 102 (ORF of the Jar1 protein, A. thaliana), SEQ ID NO: 103 (ORF of the Kp1 protein, A. thaliana), SEQ ID NO: 104 (ORF of the Lrx2 protein, A. thaliana), SEQ ID NO: 105 (ORF of the Mapkkk3 protein, A. thaliana), SEQ ID NO: 106 (ORF of the Mapkkk5 protein, A. thaliana), SEQ ID NO: 107 (ORF of the Mfp2 protein, A. thaliana), SEQ ID NO: 108 (ORF of the Mrb1 protein, A. thaliana), SEQ ID NO: 109 (ORF of the Nsp1 protein, M. truncatula), SEQ ID NO: 110 (ORF of the Nsp1 protein, A. thaliana), SEQ ID NO: 111 (ORF of the Nsp1 protein, B. distachyon), SEQ ID NO: 112 (ORF of the Nsp1 protein, G. max), SEQ ID NO: 113 (ORF of the Nsp1 protein, G. max), SEQ ID NO: 114 (ORF of the Nsp1 protein, O. sativa), SEQ ID NO: 115 (ORF of the Nsp1 protein, S. lycopersicum), SEQ ID NO: 116 (ORF of the Nsp1 protein, S. lycopersicum), SEQ ID NO: 117 (ORF of the Nsp1 protein, Z. mays), SEQ ID NO: 118 (ORF of the Nsp1 protein, Z. mays), SEQ ID NO: 119 (ORF of Nsp1 protein, Z. mays), SEQ ID NO: 120 (ORF of Nsp1 protein, Z. mays), SEQ ID NO: 121 (ORF of Nsp1 protein, B. rapa), SEQ ID NO: 122 (ORF of Nsp1 protein, H. vulgare), SEQ ID NO: 123 (ORF of Nsp1 protein, H. vulgare), SEQ ID NO: 124 (ORF of Nsp1 protein, H. vulgare), SEQ ID NO: 125 (ORF of the Nsp1 protein, H. vulgare), SEQ ID NO: 126 (ORF of the Nsp1 protein, H. vulgare), SEQ ID NO: 127 (ORF of the Nsp1 protein, H. vulgare), SEQ ID NO: 128 (ORF of the Nsp1 protein, H. vulgare), SEQ ID NO: 129 (ORF of the Nsp1 protein, S. tuberosum), SEQ ID NO: 130 (ORF of the Nsp1 protein, S. tuberosum), SEQ ID NO: 131 (ORF of the Nsp1 protein, T. aestivum), SEQ ID NO: 132 (ORF of the Nsp1 protein, T. aestivum), SEQ ID NO: 133 (ORF of the Nsp1 protein, L. perenne), SEQ ID NO: 134 (ORF of the Nsp1 protein, L. perenne), SEQ ID NO: 135 (ORF of the Pds protein, A. thaliana), SEQ ID NO: 136 (ORF of the Pen3 protein, A. thaliana), SEQ ID NO: 137 (ORF of the Phyb protein, A. thaliana), SEQ ID NO: 138 (ORF of the Pif3 protein, A. thaliana), SEQ ID NO: 139 (ORF of the Pizza protein, A. thaliana), SEQ ID NO: 140 (ORF of the Ppox1 protein, A. thaliana), SEQ ID NO: 141 (ORF of the Ppox2 protein, A. thaliana), SEQ ID NO: 142 (ORF of the Prp39 protein, A. thaliana), SEQ ID NO: 143 (ORF of the PsbA protein, A. thaliana), SEQ ID NO: 144 (ORF of the Pskr1 protein, A. thaliana), SEQ ID NO: 145 (ORF of the Rd21 protein, A. thaliana), SEQ ID NO: 146 (ORF of the Ring1 protein, A. thaliana), SEQ ID NO: 147 (ORF of the Ros1 protein, A. thaliana), SEQ ID NO: 148 (ORF of the Rpt4a protein, A. thaliana), SEQ ID NO: 149 (ORF of the Sfr6 protein, A. thaliana), SEQ ID NO: 150 (ORF of the Shr protein, A. thaliana), SEQ ID NO: 151 (ORF of the Shy2 protein, A. thaliana), SEQ ID NO: 152 (ORF of the Skl protein, M. truncatula), SEQ ID NO: 153 (ORF of the Sps1 protein, A. thaliana), SEQ ID NO: 154 (ORF of the Spt protein, A. thaliana), SEQ ID NO: 155 (ORF of the Stn8 protein, A. thaliana), SEQ ID NO: 156 (ORF of the Tap46 protein, A. thaliana), SEQ ID NO: 157 (ORF of the Topp6 protein, A. thaliana), SEQ ID NO: 158 (ORF of the TubB6 protein, A. thaliana), SEQ ID NO: 159 (ORF of the TubB8 protein, A. thaliana), SEQ ID NO: 160 (ORF of the Uba1a protein, A. thaliana), SEQ ID NO: 161 (ORF of the Vim3 protein, A. thaliana), SEQ ID NO: 381 (ORF of the Sgr1 protein, A. thaliana), SEQ ID NO: 382 (ORF of the Abi5 protein, A. thaliana), SEQ ID NO: 383 (ORF of the Hsp101 protein, A. thaliana), SEQ ID NO: 384 (ORF of the Rh10 protein, M. truncatula) and SEQ ID NO: 385 (ORF of the Wus protein, A. thaliana).

In one embodiment, the invention relates to the method for preparing and determining a cPEP as described above, wherein said protein is encoded by an ORF comprising a nucleic acid sequence having at least 80% identity, preferably at least 90% identity, with a sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method for preparing and determining a cPEP as described above, wherein the said protein is encoded by a gene comprising a nucleic acid sequence having at least 80% identity, preferably at least 90% identity, with a sequence chosen from the sequences: SEQ ID NOs: 26 to 55 (Cpk3). In particular, the invention relates to the method for preparing and determining a cPEP as described above, wherein said protein is encoded by a gene comprising a nucleic acid sequence having at least 80% identity, preferably at least 90% identity, with a sequence chosen from the sequences: SEQ ID NOs: 61 to 79 (Dcl1). In particular, the invention relates to the method for preparing and determining a cPEP as described above, wherein said protein is encoded by a gene comprising a nucleic acid sequence having at least 80% identity, preferably at least 90% identity, with a sequence chosen from the sequences: SEQ ID NOs: 109 to 134 (Nsp1).

“Percentage identity” between two nucleic acid (or amino acid) sequences refers to the percentage of identical nucleotides (or amino acid residues) between the two sequences to be compared, obtained after the best alignment. This percentage is purely statistical and the differences between the two sequences are randomly distributed over the entire length of the sequences. The best alignment (or optimal alignment) is the alignment for which the percentage of identity between the two sequences to be compared, as calculated below, is the highest. Sequence comparisons between two nucleic acid (or amino acid) sequences are traditionally performed by comparing these sequences after they have been optimally aligned, said comparison being performed by segment or by comparison window to identify and compare local regions of sequence similarity. The optimal alignment of sequences for comparison can be carried out manually or using algorithms and software available to those skilled in the art, for example, the BLAST platform or the MatGat program (Campanella, Bitincka and Smalley, 2003).

The percentage of identity between two sequences is determined by comparing these two optimally aligned sequences by comparison window wherein the region of the sequence to be compared may comprise additions or deletions relative to the reference sequence for optimal alignment between these two sequences. The percentage identity is calculated by determining the number of identical positions for which the nucleotide (or amino acid) is identical between the two sequences, dividing this number of identical positions by the total number of positions in the comparison window and multiplying the result obtained by 100.

Within the meaning of the invention, it is understood that sequences showing “at least 80% identity” with a reference sequence may in particular show at least 80%, 81%, 82%, 83%, 84%, 85%, 86%, 87%, 88%, 89%, 90%, 91%, 92%, 93%, 94%, 95%, 96%, 97%, 98%, 99% or 100% identity with said reference sequence.

In one embodiment, the invention relates to the method for preparing and determining a cPEP as described above, wherein the said protein is encoded by a gene comprising a nucleic acid sequence chosen from the sequences: SEQ ID NOs: 1 to 161 and 381 to 385. In one embodiment, the invention also relates to the method for preparing and determining a cPEP as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method for preparing and determining a cPEP as described above, wherein said protein is encoded by a gene comprising one selected from the sequences: SEQ ID NOs: 26 to 55 (Cpk3). In particular, the invention relates to the method for preparing and determining a cPEP as described above, wherein said protein is encoded by a gene comprising one selected from the sequences: SEQ ID NOs: 61 to 79 (Dcl1). In particular, the invention relates to the method for preparing and determining a cPEP as described above, wherein said protein is encoded by a gene comprising one selected from the sequences: SEQ ID NOs: 109 to 134 (Nsp1).

In another embodiment, the invention relates to the method for preparing and determining a cPEP as described above, wherein the sequence of said peptide is chosen from the sequences: SEQ ID NO: 162 (cPEPcpk3), SEQ ID NO: 163 (cPEPdcl1), SEQ ID NO: 164 (cPEPnsp1_3), SEQ ID NO: 165 (cPEPnsp1_1), SEQ ID NO: 166 (cPEPnsp1_2), SEQ ID NO: 167 (cPEPnsp1_4), SEQ ID NO: 168 (cPEPnsp1_5), SEQ ID NO: 169 (cPEPnsp1 5aa), SEQ ID NO: 170 (cPEPnsp1 20aa), SEQ ID NO: 171 (cPEPnsp1 30aa), SEQ ID NO: 172 (cPEPnsp1 40aa), SEQ ID NO: 173 (cPEPnsp1 60aa), SEQ ID NO: 174 (cPEPnsp1 80aa), SEQ ID NO: 175 (cPEPgapc), SEQ ID NO: 176 (cPEPbri1), SEQ ID NO: 177 (cPEPbak1), SEQ ID NO: 178 (cPEPshy2), SEQ ID NO: 179 (cPEPpizza), SEQ ID NO: 180 (cPEPmrb1), SEQ ID NO: 181 (cPEPtap46), SEQ ID NO: 182 (cPEPspt), SEQ ID NO: 183 (cPEPga2ox7), SEQ ID NO: 184 (cPEPphyb), SEQ ID NO: 185 (cPEPhag5), SEQ ID NO: 186 (cPEPshr), SEQ ID NO: 187 (cPEPmapkkk3), SEQ ID NO: 188 (cPEPmapkkk5_1), SEQ ID NO: 189 (cPEPmapkkk5_2), SEQ ID NO: 190 (cPEPring1), SEQ ID NO: 191 (cPEPros1), SEQ ID NO: 192 (cPEPjar1), SEQ ID NO: 193 (cPEPcoi1), SEQ ID NO: 194 (cPEPabcg34), SEQ ID NO: 195 (cPEPagb1), SEQ ID NO: 196 (cPEPwus), SEQ ID NO: 197 (AhEIN2), SEQ ID NO: 198 (AhBRI1), SEQ ID NO: 199 (AhBAK1), SEQ ID NO: 200 (AtEIN2cPEP1), SEQ ID NO: 201 (AtEIN2cPEP2), SEQ ID NO: 202 (AtEIN2cPEP3), SEQ ID NO: 203 (AtEIN2cPEP13), SEQ ID NO: 204 (cPEPein2_1), SEQ ID NO: 205 (cPEPein2_2), SEQ ID NO: 206 (cPEPein2_3), SEQ ID NO: 404 (cPEPhsp101), SEQ ID NO: 406 (cPEPabi5), SEQ ID NO: 407 (cPEPsgr1), SEQ ID NO: 408 (cPEPhsp101), SEQ ID NO: 409 (cPEPmrb1), SEQ ID NO: 410 (cPEPshy2), SEQ ID NO: 411 (cPEPsk1), SEQ ID NO: 412 (cPEPrh10), SEQ ID NO: 413 (cPEpjar1), SEQ ID NO: 414 (cPEPbak1), SEQ ID NO: 415 (cPEPbri1), SEQ ID NO: 416 (cPEPwus) and SEQ ID NO: 417 (cPEPein2).

In particular, the invention relates to the method for preparing and determining a cPEP as described above, wherein the sequence of the said peptide is chosen from the sequences: SEQ ID NO: 162, SEQ ID NO: 163 and SEQ ID NOs: 164 to 174. In particular, the invention relates to the method for preparing and determining a cPEP as described above, wherein the sequence of said peptide is the sequence SEQ ID NO: 162. In particular, the invention relates to the method for preparing and determining a cPEP as described above, wherein the sequence of said peptide is the sequence: SEQ ID NO: 163. In particular, the invention relates to the method for preparing and determining a cPEP as described above, wherein the sequence of said peptide is selected from the sequences: SEQ ID NOs: 164 to 174.

In a second aspect, the above invention relates to a cPEP as obtained by implementing the method as described above. According to this same aspect, the invention also relates to a cPEP, from 4 to 70 amino acids, in particular from 4 to 41 amino acids, the amino acid sequence of which corresponds to the translation via the genetic code of a fragment (not naturally translated) of a nucleic acid sequence naturally translated on an mRNA in a plant cell which corresponds to the open reading frame of a protein encoded by the said mRNA, said cPEP being capable of modulating the accumulation of said protein in the plant cell and not being capable of modulating the accumulation of the mRNA encoding said protein.

In one embodiment, the invention relates to cPEP as previously described, said fragment having a size of 3n nucleotides, n being comprised:
from 4 to 41;
from 5 to 40;
from 7 to 20; or
from 8 to 15.

In other words, the invention relates to cPEP as described above, said cPEP comprising 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

In particular, the invention relates to cPEP as described above, said cPEP comprising from 4 to 41 amino acids. In particular, the invention relates to cPEP as described above, said cPEP comprising from 5 to 40 amino acids. In particular, the invention relates to cPEP as described above, said cPEP comprising from 7 to 20 amino acids. In particular, the invention also relates to cPEP as described above, said cPEP comprising from 8 to 15 amino acids.

In one embodiment, the invention relates to cPEP as described above, wherein the size of said cPEP is smaller than that of said protein.

In one embodiment, the invention relates to the cPEP as described above, said fragment lacking:
the initiation codon AUG encoding an initiator methionine; or
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention therefore relates to the cPEP as described above, said fragment comprising an AUG initiation codon encoding an initiator methionine and lacking a STOP codon chosen from the codons: UAG, UGA and UAA. The invention also relates to the cPEP as described above, said fragment lacking an initiation codon AUG encoding an initiator methionine and comprising a STOP codon chosen from codons: UAG, UGA and UAA.

In one embodiment, the invention relates to cPEP as described above, said fragment lacking:
the AUG initiation codon encoding a initiator methionine; and
a STOP codon chosen from: UAG, UGA and UAA,
   and wherein said fragment being selected from:
either in the same reading frame as the open reading frame encoding said protein;
or in a reading frame shifted by one or two nucleotides relative to the open reading frame encoding said protein.

In one embodiment, the invention relates to the cPEP as described above, wherein the translation of the mRNA fragment is carried out in the same reading frame as the open reading frame of the nucleic acid sequence naturally translated in said plant cell. In other words, the invention relates to the cPEP as described above, wherein the translation of the mRNA fragment is carried out in the reading frame determined by the initiation codon of the open reading frame of the said protein.

In one embodiment, the invention relates to the cPEP as described above, wherein the translation of the mRNA fragment is carried out in a reading frame different from the open reading frame of the nucleic acid sequence naturally translated in said plant cell. In particular, the invention relates to the cPEP as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the cPEP as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the cPEP as described above, wherein the translation of the mRNA fragment is carried out in a reading frame shifted by one (or two nucleotides at 5′) or two nucleotides at 3′ (or one nucleotide at 5′) with respect to the initiation codon of the open reading frame of the nucleic acid sequence naturally translated in said plant cell.

In one embodiment, the invention relates to a cPEP, from 4 to 70 amino acids, in particular from 4 to 41 amino acids, the amino acid sequence of which corresponds to the translation via the genetic code of a fragment of a nucleic acid sequence of a protein mRNA, said nucleic acid sequence comprising two contiguous parts:
a part located within a nucleic acid sequence known to be non-coding (i.e. not naturally translated, e.g. 3′UTR or 5′UTR); and
a part located within a nucleic acid sequence considered to be coding (i.e. naturally translated, e.g. exon),
   said fragment having a size of 3n nucleotides, n being from 4 to 70, in particular n being from 4 to 41, and said cPEP being capable of modulating the accumulation of said protein in a plant cell and not being capable of modulating the accumulation of the mRNA encoding said protein.

In one embodiment, the invention relates to cPEP as previously described, wherein said cPEP is capable of increasing the accumulation of said protein in said plant cell.

In one embodiment, the invention relates to cPEP as previously described, wherein said cPEP is capable of decreasing the accumulation of said protein in said plant cell.

In one embodiment, the invention relates to cPEP as described above, wherein said cPEP is a synthetic peptide.

In one embodiment, the invention relates to cPEP as described above, wherein said cPEP is an isolated peptide.

In one embodiment, the invention relates to cPEP as described previously, wherein said cPEP is a recombinant peptide.

In one embodiment, the invention relates to cPEP as described above, said cPEP being a hydrophobic peptide or a hydrophilic peptide.

In one embodiment, the invention relates to cPEP as described above, wherein said protein is naturally present in said plant cell.

In one embodiment, the invention relates to cPEP as described above, wherein said protein is not naturally present in said plant cell. In particular, the invention relates to cPEP as described above, wherein said protein is encoded by a transgene artificially introduced into said plant cell. In particular, the invention relates to cPEP as described above, wherein said protein is encoded by a vector artificially introduced into said plant cell.

In one embodiment, the invention relates to cPEP as described above, wherein the said plant cell (i.e. the one wherein it is desired to modulate the accumulation of a protein) belongs to a plant species chosen from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (trefoil), Medicago sativa (alfalfa), Medicago truncatula (lucerne), Nicotiana benthamiana (tobacco), Oryza sativa (rice), Pisum sativum (pea), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In one embodiment, the invention relates to cPEP as described above, wherein said plant cell is an algal cell.

In one embodiment, the invention relates to cPEP as described above, wherein said protein is encoded by a gene selected from: Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2 h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexo1, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Skl, Sps1, Spt, Stn8, Tap46, Topp6, TubB6, TubB8, Uba1a, Vim3, Sgr1, Abi5, Hsp101, Rh10 and Wus.

In particular, the invention relates to cPEP as described above, wherein the said protein is encoded by a gene chosen from the following genes: Cpk3, Dcl and Nsp1. In particular, the invention relates to cPEP as described above, wherein the said protein is encoded by the Cpk3 gene. In particular, the invention relates to cPEP as described above, wherein the said protein is encoded by the Dcl gene. In particular, the invention relates to cPEP as described above, wherein the said protein is encoded by the Nsp1 gene.

In one embodiment, the invention relates to cPEP as described above, wherein said protein is encoded by a gene comprising a nucleic acid sequence having at least 80% identity, preferably at least 90% identity, with a sequence chosen from the sequences: SEQ ID NOs: 1 to 161 and 381 to 385.

In particular, the invention relates to the cPEP as described above, wherein the said protein is encoded by a gene comprising a nucleic acid sequence having at least 80% identity, preferably at least 90% identity, with a sequence chosen from the sequences: SEQ ID NOs: 26 to 55 (Cpk3), SEQ ID NOs: 61 to 79 (Dcl1) and SEQ ID NOs: 109 to 134 (Nsp1). In particular, the invention also relates to cPEP as described above, wherein the said protein is encoded by a gene comprising a nucleic acid sequence having at least 80% identity, preferably at least 90% identity, with a sequence chosen from the sequences: SEQ ID NOs: 26 to 55 (Cpk3). In particular, the invention also relates to cPEP as described above, wherein the said protein is encoded by a gene comprising a nucleic acid sequence having at least 80% identity, preferably at least 90% identity, with a sequence chosen from the sequences: SEQ ID NOs: 61 to 79 (Dcl1). In particular, the invention also relates to cPEP as described above, wherein the said protein is encoded by a gene comprising a nucleic acid sequence exhibiting at least 80% identity, preferably at least 90% identity, with a sequence chosen from the sequences: SEQ ID NOs: 109 to 134 (Nsp1).

In one embodiment, the invention relates to the cPEP as described above, wherein the said protein is encoded by a gene comprising a nucleic acid sequence chosen from the sequences: SEQ ID NOs: 1 to 161 and 381 to 385. The invention relates in particular to cPEP as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to cPEP as described above, wherein said protein is encoded by a gene comprising one selected from the sequences: SEQ ID NOs: 26 to 55 (Cpk3). In particular, the invention relates to cPEP as described above, wherein said protein is encoded by a gene comprising one selected from the sequences: SEQ ID NOs: 61 to 79 (Dcl1). In particular, the invention relates to cPEP as described above, wherein said protein is encoded by a gene comprising one selected from the sequences: SEQ ID NOs: 109 to 134 (Nsp1).

In another embodiment, the invention relates to cPEP as described above, wherein the sequence of said peptide is chosen from the sequences: SEQ ID NOs: 162 to 206, 404 and 406 to 417.

In particular, the invention relates to the cPEP as described above, wherein the sequence of said peptide is chosen from the sequences: SEQ ID NO: 162, SEQ ID NO: 163 and SEQ ID NOs: 164 to 174. In particular, the invention relates to the cPEP as described above, wherein the sequence of said peptide is the sequence SEQ ID NO: 162. In particular, the invention relates to the cPEP as described above, wherein the sequence of said peptide is the sequence: SEQ ID NO: 163. In particular, the invention relates to the cPEP as described above, wherein the sequence of the said peptide is chosen from the sequences: SEQ ID NOs: 164 to 174.

In one embodiment, the invention relates to cPEP as described above, wherein said protein is involved in at least one plant phenotype selected from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

In the invention, a cPEP can be fused or linked to one or more molecules that facilitate entry of the cPEP into the cell. These molecules include penetrating peptides (Numata, K., et al.

Library screening of cell-penetrating peptide for BY-2 cells, leaves of Arabidopsis, tobacco, tomato, poplar, and rice callus. Sci Rep 8, 10966 (2018).) and palmitic acid. Penetrating peptide” (hereinafter CPP) refers to small peptides that penetrate cellular lipid bilayers or destabilise cell membranes. CPPs can be classified into three groups: cationic, amphipathic and hydrophobic. In particular:
Cationic CPPs contain many positively charged amino acids, such as lysine (Lys) and arginine (Arg);
amphipathic CPPs are generally composed of an alternating sequence of polar and non-polar amino acids; and
hydrophobic CPPs are composed of non-polar amino acids with relatively low net charges.

In one embodiment, the invention relates to cPEP as described above, said cPEP being fused to a peptide facilitating its entry into the plant cell. In particular, the invention relates to cPEP as described above, said cPEP being fused to a penetrating peptide.

In one embodiment, the invention relates to cPEP as described above, said cPEP being fused at the N-terminus or at the C-terminus with said peptide facilitating its entry into the plant cell.

In particular, the invention relates to cPEP as described above, said cPEP being fused at the N-terminus or at the C-terminus with said penetrating peptide.

In one embodiment, the invention relates to cPEP as described above, said cPEP being fused with:
TAT peptide (SEQ ID NO: 380);
penetratin;
a polyhistidine peptide (in particular a peptide with at least 4 histidine residues);
or
a polyarginine peptide (in particular a peptide with 4 arginine residues).

In one embodiment, the invention relates to cPEP as described above, said cPEP being linked to one or more palmitic acid molecules.

In one embodiment, the invention relates to cPEP as described above, said cPEP being linked at the N-terminus or at the C-terminus to one or more palmitic acid molecules.

On this point, it should be noted that the quantity of cPEP required to modulate the accumulation of a protein may vary depending on whether or not the cPEP is modified with one of the molecules facilitating its cellular penetration.

In a third aspect, the above invention relates to a nucleic acid encoding a cPEP as described above. According to this same aspect, the invention also has as its object a nucleic acid of 3n nucleotides, which nucleic acid corresponds to a fragment of a nucleic acid sequence naturally translated on an mRNA in a plant cell which corresponds to the open reading frame of a protein encoded by said mRNA.

In one embodiment, the invention relates to the nucleic acid described above, said fragment lacking:
the initiation codon AUG encoding an initiator methionine; or
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention therefore relates to the nucleic acid as described above, said fragment comprising an AUG start codon encoding a initiator methionine and lacking a STOP codon chosen from the codons: UAG, UGA and UAA. The invention also relates to the nucleic acid as described above, the said fragment lacking an initiation codon AUG encoding an initiator methionine and comprising a STOP codon chosen from the codons: UAG, UGA and UAA.

In one embodiment, the invention relates to the nucleic acid described above, said fragment lacking:
the initiation codon AUG encoding an initiator methionine; and
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention relates to the nucleic acid as described above, wherein the translation of the mRNA fragment is carried out in the same reading frame as the open reading frame of the nucleic acid sequence naturally translated in said plant cell. In other words, the invention relates to the nucleic acid as described above, wherein the translation of the mRNA fragment is carried out in the reading frame determined by the initiation codon of the open reading frame of the said protein.

In one embodiment, the invention relates to the nucleic acid as described above, wherein the translation of the mRNA fragment is carried out in a reading frame different from the open reading frame of the nucleic acid sequence naturally translated in said plant cell. In particular, the invention relates to the nucleic acid as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the nucleic acid as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the nucleic acid as described above, wherein the translation of the mRNA fragment is carried out in a reading frame shifted by one (or two nucleotides at 5′) or two nucleotides at 3′ (or one nucleotide at 5′) with respect to the initiation codon of the open reading frame of the nucleic acid sequence naturally translated in said plant cell.

In one embodiment, the invention relates to a nucleic acid of 3n nucleotides, which nucleic acid corresponds to a fragment of a nucleic acid sequence of a protein mRNA, said nucleic acid sequence comprising two contiguous parts:
a part located within a nucleic acid sequence known to be non-coding (i.e. not naturally translated, e.g. 3′UTR or 5′UTR); and
a part located within a nucleic acid sequence considered to be coding (i.e. naturally translated, e.g. exon).

In particular, the invention relates to nucleic acid as described above, where n is:
from 4 to 70;
from 4 to 41;
from 5 to 40;
from 7 to 20; or
from 8 to 15.

In another aspect, the above invention relates to a composition comprising a cPEP as described above as active substance.

In one embodiment, the invention relates to a composition comprising a cPEP as active substance, said cPEP:
having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of a protein encoded by said mRNA; and
being capable of modulating the accumulation of said protein in the plant cell and not being capable of modulating the accumulation of the mRNA encoding said protein.

In one embodiment, the invention relates to the composition as described above, wherein said fragment is devoid of:
the initiation codon AUG encoding an initiator methionine; or
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention therefore relates to the composition as described above, wherein the said fragment comprises an AUG initiation codon encoding an initiator methionine and lacks a STOP codon chosen from the codons: UAG, UGA and UAA. The invention also relates to the composition as described above, wherein the said fragment lacks an initiation codon AUG encoding an initiator methionine and comprises a STOP codon chosen from codons: UAG, UGA and UAA.

In one embodiment, the invention relates to the composition as described above, wherein said fragment is devoid of:
the initiation codon AUG encoding an initiator methionine; and
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention relates to the composition as described above, wherein the translation of the mRNA fragment is carried out in the same reading frame as the open reading frame of the nucleic acid sequence naturally translated in the said plant cell. In other words, the invention relates to the composition as described above, wherein the translation of the mRNA fragment is carried out in the reading frame determined by the initiation codon of the open reading frame of the said protein.

In one embodiment, the invention relates to the composition as described above, wherein the translation of the mRNA fragment is carried out in a reading frame different from the open reading frame of the nucleic acid sequence naturally translated in the said plant cell. In particular, the invention relates to the composition as described above, wherein the translation of the mRNA fragment is carried out in a reading frame shifted by one nucleotide at 3′ (or by two nucleotides at 5′) with respect to the open reading frame of the nucleic acid sequence naturally translated in the said plant cell. In particular, the invention also relates to the composition as described above, wherein the translation of the mRNA fragment is carried out in a reading frame shifted by two nucleotides 3′ (or one nucleotide 5′) with respect to the open reading frame of the nucleic acid sequence naturally translated in the said plant cell. In other words, the invention relates to the composition as described above, wherein the translation of the mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in the said plant cell.

In one embodiment, the invention relates to a composition comprising a cPEP as active substance, said cPEP:
having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of a protein encoded by said mRNA, said fragment having a size of 3n nucleotides, n being from 4 to 70, in particular n being from 4 to 41, and said nucleic acid sequence comprising two contiguous parts:
   a part located within a nucleic acid sequence known to be non-coding (i.e. not naturally translated, e.g. 3′UTR or 5′UTR); and
   a part located within a nucleic acid sequence known to be coding (i.e. naturally translated, e.g. exon); and
being capable of modulating the accumulation of said protein in the plant cell and not being capable of modulating the accumulation of the mRNA encoding said protein.

In one embodiment, the invention relates to the composition as described above, wherein the said cPEP is at a concentration from 10⁻⁹ M to 10⁻³ M. On this point, it should be noted on the one hand that the composition of the invention does not exist in the natural state, and this is all the more true as such a concentration of cPEP cannot exist within a plant cell. Furthermore, by “concentration from 10⁻⁹ M to 10⁻³ M”, is meant that the concentration of cPEP can be from 10⁻⁹ to 10⁻⁴ M, from 10⁻⁸ to 10⁻⁴ M, from 10⁻⁹ to 10⁻⁵ M, from 10⁻⁸ to 10⁻⁵ M, or from 5 μM to 500 μM, from 30 μM to 70 μM, or even 50 μM.

In particular, the invention relates to the composition as described above, wherein the said cPEP is at a concentration from 10⁻⁹ and 10⁻⁴ M, from 10⁻⁸ to 10⁻⁴ M, from 10⁻⁹ to 10⁻⁵ M or from 10⁻⁸ to 10⁻⁵ M. In particular, the invention relates to the composition as described above, wherein the said cPEP is at a concentration from 5 μM to 500 μM or from 30 μM to 70 μM. In particular, the invention relates to the composition as described above, wherein the said cPEP is at a concentration of 50 μM. Alternatively, this concentration may be 10⁻⁹ M, 10⁻⁸ M, 10⁻⁷ M, 10⁻⁶ M, 10⁻⁵ M or 10⁻⁴ M.

In view of the above, it is understood that the invention also relates to the composition comprising a cPEP as active substance, said cPEP:
having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of a protein encoded by said mRNA;
being capable of modulating the accumulation of said protein in the plant cell and not being capable of modulating the accumulation of the mRNA encoding said protein; and
being in particular at a concentration from 5 μM to 500 μM or from 30 μM to 70 μM, or being in particular at a concentration of 50 μM.

It should be noted that “composition comprising a cPEP” means that the composition of the invention comprises at least one cPEP. In other words, a mixture of cPEPs is conceivable, said cPEPs being able to target the same protein or several proteins depending on the nucleic acid fragment from which they are derived. In this respect, the aforementioned concentrations relate either to the mixture of cPEPs as such, or to each of the cPEPs in the said mixture, the said cPEPs possibly being at the same concentration or possibly being at different concentrations from those mentioned above.

In one embodiment, the invention relates to the composition as described above, said composition being a phytopharmaceutical composition, a herbicidal composition or a coating composition, in particular said coating composition further comprising at least one fixing agent.

In particular, the invention relates to the composition as described above, said composition being a phytopharmaceutical composition. In particular, the invention relates to the composition as described above, the said composition being a herbicidal composition. In particular, the invention relates to the composition as described above, said composition being a coating composition. Preferably, the invention relates to the composition as described above, the said composition being a coating composition additionally comprising at least one fixing agent.

In one embodiment, the invention relates to the composition as described above, said composition further comprising at least one solvent. Preferably, the said solvent is chosen from: acetone, acetonitrile, acetic acid, formic acid, dimethyl adipate, benzyl acetate, bi-butyl carbonate, dimethyl sulphoxide (DMSO), water, dimethyl glutarate, ammonium hydroxide, iso-butanol, iso-propanol, diethyl hexyl lactate, light aromatic naphtha solvent, heavy aromatic naphtha solvent, diethyl succinate and mixtures thereof (e.g. mixture [water; acetic acid]). g. mixture [water; acetic acid]; [acetonitrile; acetic acid], [water, acetonitrile; acetic acid], [water; DMSO], [water; acetonitrile] or [water; ammonium hydroxide]).

The solubility properties of cPEPs are determined in particular by their amino acid composition.

Hydrophilic cPEPs can be solubilised and packaged in aqueous solutions, such as water.

Hydrophobic cPEPs can be solubilised and packaged in solvents, such as organic solvents.

For treatment of plants with cPEPs, the organic solvents are non-toxic for the plants in small quantities, i.e. they have no deleterious effect on the development of the plant. By way of example, the organic solvents may be those mentioned above and in particular selected from acetonitrile and acetic acid.

As mentioned above, cPEPs can also be solubilised and packaged in solvent mixtures such as, for example, an organic solvent mixture [acetonitrile; acetic acid], a mixture [water; DMSO] in a volume:volume ratio from 99:1 to 1:99, a mixture [water; acetonitrile] in a volume:volume ratio from 99:1 to 1:99 or a mixture [water; ammonium hydroxide]:volume ratio from 99:1 to 1:99, a [water; acetonitrile] mixture in a volume:volume ratio from 99:1 to 1:99 or a [water; ammonium hydroxide] mixture in a volume:volume ratio from 99:1 to 99.9:0.1. The cPEPs can also be solubilised in a solution comprising 50% acetonitrile, 10% acetic acid and 40% water (v/v/v).

In one embodiment, the invention relates to the composition as described above, said composition further comprising at least one diluent.

In one embodiment, the invention relates to the composition as described above, said composition further comprising at least one adjuvant.

In one embodiment, the invention relates to the composition as described above, said composition further comprising at least one fixing agent.

By “fixing agent” is meant a chemical or natural agent which enables the composition of the invention to adhere to a plant seed so as to coat the said plant seed. It also means a substance that makes it possible to apply and hold the active substance(s) on the seed. Available fixing agents include carboxymethyl cellulose (CMC) and gum arabic. In addition, and without limitation, a fixing agent may comprise organic solvents, water, dispersants, emulsifiers, surfactants, wetting agents and dyes.

In one embodiment, the invention relates to the composition as described above, said composition further comprising at least one plant nutrient. In particular, the invention relates to the composition as described above, the said composition additionally comprising at least one fixing agent and at least one plant nutrient.

By “plant nutrient” is meant an element assimilated by the plant to enable it to develop. By no means restrictive, a plant nutrient can be chosen from: nitrogen, phosphorus, potassium, calcium, magnesium, sulphur, manganese, iron, copper, boron, zinc, molybdenum and mixtures thereof.

In view of the foregoing, it is understood that another aspect of the invention relates to a coated seed comprising a plant seed, said plant seed being coated with a coating composition as previously described.

The coating can be produced using methodes conventionally used in the food industry and can be obtained by using a material capable of disintegrating in a solvent or in the earth, such as a binder or clay.

According to the invention, the coating can be used to confer particular properties on a seed in combination with a cPEP, such as improved growth or resistance to certain biotic or abiotic stresses.

In one embodiment, the invention relates to the coated seed as described above, wherein said plant seed to a plant species selected from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In one embodiment, the invention relates to coated seed as described above, said seed being treated by dipping in a cPEP-containing composition. During dipping, the seed is then totally or partially immersed in a composition containing a cPEP.

In another aspect, the above invention relates to a use of a cPEP as a plant protection agent to modulate the accumulation of a protein in a plant cell, said cPEP having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of said protein encoded by said mRNA, said cPEP being capable of modulating the accumulation of said protein in the plant cell and not being capable of modulating the accumulation of the mRNA encoding said protein.

In one embodiment, the invention relates to the use of a cPEP as described above, wherein said fragment is devoid of:
the initiation codon AUG encoding an initiator methionine; or
a STOP codon chosen from: UAG, UGA and UAA,
   and said fragment being selected from:
either the same open reading frame as that encoding the said protein;
or in an open reading frame shifted by one or two nucleotides relative to that encoding said protein.

In one embodiment, the invention therefore relates to the use of a cPEP as described above, wherein said fragment comprises an AUG initiation codon encoding an initiator methionine and lacks a STOP codon chosen from the codons: UAG, UGA and UAA. The invention also relates to the use of a cPEP as described above, wherein said fragment is devoid of an AUG start codon encoding a initiator methionine and comprises a STOP codon selected from codons: UAG, UGA and UAA.

In one embodiment, the invention relates to the use of a cPEP as described above, wherein said fragment is devoid of:
the AUG initiation codon encoding a initiator methionine; and
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention relates to the use of a cPEP as described above, wherein the translation of the mRNA fragment is carried out in the same reading frame as the open reading frame of the nucleic acid sequence naturally translated in said plant cell. In other words, the invention relates to the use of a cPEP as described above, wherein the translation of the mRNA fragment is carried out in the reading frame determined by the initiation codon of the open reading frame of the said protein.

In one embodiment, the invention relates to the use of a cPEP as described above, wherein the translation of the mRNA fragment is carried out in a reading frame different from the open reading frame of the nucleic acid sequence naturally translated in said plant cell. In particular, the invention relates to the use of a cPEP as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the use of a cPEP as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the use of a cPEP as described above, wherein the translation of the mRNA fragment is carried out in a reading frame shifted by one (or two nucleotides 5′) or two nucleotides 3′ (or one nucleotide 5′) with respect to the initiation codon of the open reading frame of the nucleic acid sequence naturally translated in said plant cell.

In one embodiment, the invention relates to the use of a cPEP as a plant protection agent to modulate the accumulation of a protein in a plant cell, said cPEP having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of said protein encoded by said mRNA, said fragment having a size of 3n nucleotides, n being from 4 to 70, in particular n being from 4 to 41, and said nucleic acid sequence comprising two contiguous parts:
a part located within a nucleic acid sequence known to be non-coding (i.e. not naturally translated, e.g. intron); and
a part located within a nucleic acid sequence considered to be coding (i.e. naturally translated, e.g. exon);
said cPEP being capable of modulating the accumulation of said protein in the plant cell and not being capable of modulating the accumulation of the mRNA encoding said protein.

In one embodiment, the invention relates to the use of a cPEP as described above to increase the accumulation of said protein in the plant cell. The presence of the cPEP causes the amount of said protein in the treated plant cell to be greater than that in an untreated plant cell.

In one embodiment, the invention relates to the use of a cPEP as described above to decrease (inhibit) the accumulation of said protein in the plant cell. The presence of the cPEP causes the amount of said protein in the treated plant cell to be less than that in an untreated plant cell.

In one embodiment, the invention relates to the use of a cPEP as described above, wherein said cPEP is produced outside said plant cell prior to being introduced into said plant cell.

In one embodiment, the invention relates to the use of a cPEP as described above, wherein said cPEP is a synthetic peptide.

In one embodiment, the invention relates to the use of a cPEP as described above, wherein said cPEP is an isolated peptide.

In one embodiment, the invention relates to the use of a cPEP as described above, wherein said cPEP is a recombinant peptide.

In one embodiment, the invention relates to the use of a cPEP as described above, wherein said cPEP is a hydrophobic peptide or a hydrophilic peptide.

In one embodiment, the invention relates to the use of a cPEP as described above, wherein said cPEP is introduced into said plant cell in the form of a nucleic acid encoding said cPEP.

In particular, the invention relates to the use of a cPEP as described above, wherein said cPEP is introduced into said plant cell in the form of a nucleic acid encoding said cPEP and comprising the means for expressing it.

In one embodiment, the invention relates to the use of a cPEP as described above, wherein said protein is naturally present in said plant cell.

In one embodiment, the invention relates to the use of a cPEP as described above, wherein said protein is not naturally present in said plant cell. In particular, the invention relates to the use of a cPEP as described above, wherein said protein is encoded by a transgene artificially introduced into said plant cell. In particular, the invention relates to the use of a cPEP as described above, wherein said protein is encoded by a vector artificially introduced into said plant cell.

In one embodiment, the invention relates to the use of a cPEP as described above, wherein the accumulation of said protein is determined via the implementation of a technique selected from: Western blot, measurement of enzymatic activity, mass spectrometry and translational fusion. In particular, the invention relates to the use of a cPEP as described above, wherein the accumulation of said protein is determined via the implementation of a Western blot.

In one embodiment, the invention relates to the use of a cPEP as described above, wherein said cPEP has a size from 4 to 41 amino acids, from 5 to 40 amino acids, from 7 to 20 amino acids or more particularly a size from 8 to 15 amino acids. In particular, said cPEP has a size from 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

In one embodiment, the invention relates to the use of a cPEP as described above, wherein said plant cell belongs to a plant species selected from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (trefoil), Medicago sativa (alfalfa), Medicago truncatula (lucerne), Nicotiana benthamiana (tobacco), Oryza sativa (rice), Pisum sativum (pea), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In particular, the invention relates to the use of a cPEP as described above, wherein said plant cell is an algal cell.

In one embodiment, the invention relates to the use of a cPEP as described above, wherein said protein is encoded by a gene selected from: Aae15, Aae16, abcg11, Abdcg34, Acc1, Agb1, Als, Anac076, Apg9, ArIb1, Arr1, Arr5, Arr6, At59, Bak1, Bccp1, Bccp2, Bri1, Bzo2 h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gs/3, Hag5, Hda18, Hexo1, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Skl, Sps1, Spt, Stn8, Tap46, Topp6, TubB6, TubB8, Uba1a, Vim3, Sgr1, Abi5, Hsp101, Rh10 and Wus.

In view of the above, it is understood that in another embodiment, the invention relates to the use of a cPEP as described above, said cPEP having a size from 4 to 70 amino acids, in particular from 4 to 41 amino acids, the amino acid sequence of which corresponds to the translation via the genetic code of a non-naturally translated fragment of a nucleic acid sequence naturally translated on an mRNA in a plant cell which corresponds to the open reading frame of said protein encoded by said mRNA, said non-naturally translated fragment lacking the initiation codon AUG encoding an initiator methionine and/or a STOP codon chosen from the codons: UAG, UGA and UAA, and being chosen either in the same reading frame as the open reading frame encoding said protein, or in a reading frame shifted by one or two nucleotides relative to the open reading frame encoding said protein, and said cPEP being capable of modulating the accumulation of said protein in the plant cell and not being capable of modulating the accumulation of the mRNA encoding said protein, said protein being encoded by a gene selected from: Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2 h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexo1, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Ski, Sps1, Spt, Stn8, Tap46, Topp6, TubB6, TubB8, Uba1a, Vim3, Sgr1, Abi5, Hsp101, Rh10 and Wus.

In one embodiment, the invention relates to the use of a cPEP as described above, wherein said protein is encoded by an ORF comprising a nucleic acid sequence having at least 80% identity, preferably at least 90% identity, with a sequence selected from the sequences: SEQ ID NOs: 1 to 161 and 381 to 385.

In one embodiment, the invention relates to the use of a cPEP as described above, wherein said protein is encoded by an ORF comprising a nucleic acid sequence having at least 80% identity, preferably at least 90% identity, with a sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the use of a cPEP as described above, wherein the said protein is encoded by a gene comprising a nucleic acid sequence having at least 80% identity, preferably at least 90% identity, with a sequence chosen from the sequences: SEQ ID NOs: 26 to 55 (Cpk3). In particular, the invention relates to the use of a cPEP as described above, wherein said protein is encoded by a gene comprising a nucleic acid sequence having at least 80% identity, preferably at least 90% identity, with a sequence selected from the sequences: SEQ ID NOs: 61 to 79 (Dcl1). In particular, the invention relates to the use of a cPEP as described above, wherein said protein is encoded by a gene comprising a nucleic acid sequence having at least 80% identity, preferably at least 90% identity, with a sequence selected from the sequences: SEQ ID NOs: 109 to 134 (Nsp1).

In one embodiment, the invention relates to the use of a cPEP as described above, wherein said protein is encoded by a gene comprising a nucleic acid sequence selected from the sequences: SEQ ID NOs: 1 to 161 and 381 to 385. In one embodiment, the invention also relates to the use of a cPEP as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the use of a cPEP as described above, wherein said protein is encoded by a gene comprising one selected from the sequences: SEQ ID NOs: 26 to 55 (Cpk3). In particular, the invention relates to the use of a cPEP as described above, wherein said protein is encoded by a gene comprising one selected from the sequences: SEQ ID NOs: 61 to 79 (Dcl1). In particular, the invention relates to the use of a cPEP as described above, wherein said protein is encoded by a gene comprising one selected from the sequences: SEQ ID NOs: 109 to 134 (Nsp1).

In another embodiment, the invention relates to the use of a cPEP as described above, wherein said cPEP is selected from the sequences: SEQ ID NOs: 162 to 206, 404 and 406 to 417.

In particular, the invention relates to the use of a cPEP as described above, wherein said cPEP is selected from the sequences: SEQ ID NO: 162, SEQ ID NO: 163 and SEQ ID NOs: 164 to 174. In particular, the invention relates to the use of a cPEP as described above, wherein said cPEP is of the sequence SEQ ID NO: 162. In particular, the invention relates to the use of a cPEP as previously described, wherein said cPEP is of sequence: SEQ ID NO: 163. In particular, the invention relates to the use of a cPEP as described above, wherein said cPEP is selected from the sequences: SEQ ID NOs: 164 to 174.

In another embodiment, the invention relates to the use of a cPEP as described above, wherein said protein is involved in at least one plant phenotype selected from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

In another embodiment, the invention concerns the use of a cPEP as described above, to modulate the accumulation of a recombinant protein whose nucleic acid sequence encoding it corresponds to the fusion of the nucleic acid sequences of two distinct genes.

In particular, the coding sequence of at least one of the two genes is that of a reporter gene, for example a gene encoding a fluorescent protein (such as GFP) or a protein enabling the plant to resist a compound.

In one embodiment, the invention relates to the use of a cPEP as described above, to modulate the accumulation of a recombinant protein whose nucleic acid sequence encoding it corresponds to the fusion:
a nucleic acid sequence known to be non-coding for a first gene; and
of a nucleic acid sequence coding for a second gene, the sequence of said cPEP corresponding to the translation via the genetic code of a fragment of the nucleic acid sequence deemed non-coding of the first gene.

In another aspect, the above invention relates to a method for modulating the accumulation of a protein in a plant cell comprising a step for introduction:
a cPEP; or
of a nucleic acid encoding said cPEP and the means of expressing it, in said plant cell, the introduction of said cPEP resulting in a modulation of the quantity of said protein in said plant cell, said cPEP having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of said protein encoded by said mRNA, said cPEP being capable of modulating the accumulation of said protein in the plant cell and not being capable of modulating the accumulation of the mRNA encoding said protein.

In one embodiment, the invention relates to the method as described above, wherein said fragment is devoid of:
the AUG initiation codon encoding a initiator methionine; or
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention therefore relates to the method as described above, wherein said fragment comprises an AUG initiation codon encoding an initiator methionine and lacks a STOP codon chosen from the codons: UAG, UGA and UAA. The invention also relates to the method as described above, wherein said fragment is devoid of an initiation codon AUG encoding an initiator methionine and comprises a STOP codon selected from codons: UAG, UGA and UAA.

In one embodiment, the invention relates to the method as described above, wherein said fragment is devoid of:
the AUG initiation codon encoding a initiator methionine; and
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention relates to the method as described above, wherein the translation of the mRNA fragment is carried out in the same reading frame as the open reading frame of the nucleic acid sequence naturally translated in said plant cell. In other words, the invention relates to the method as described above, wherein the translation of the mRNA fragment is carried out in the reading frame determined by the initiation codon of the open reading frame of said protein.

In one embodiment, the invention relates to the method as described above, wherein the translation of the mRNA fragment is carried out in a reading frame different from the open reading frame of the nucleic acid sequence naturally translated in said plant cell. In particular, the invention relates to the method as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the method as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the method as described above, wherein the translation of the mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in said plant cell.

In one embodiment, the invention relates to a method for modulating the accumulation of a protein in a plant cell comprising a step for introducing:
a cPEP; or
of a nucleic acid encoding said cPEP and the means of expressing it,
   in said plant cell, the introduction of said cPEP resulting in a modulation of the quantity of said protein in said plant cell,
   said cPEP having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of said protein encoded by said mRNA, said fragment having a size of 3n nucleotides, n being from 4 to 70, in particular n being from 4 to 41, and said nucleic acid sequence comprising two contiguous parts:
a part located within a nucleic acid sequence known to be non-coding (i.e. not naturally translated, e.g. 3′UTR or 5′UTR); and
a part located within a nucleic acid sequence considered to be coding (i.e. naturally translated, e.g. exon);
   said cPEP being capable of modulating the accumulation of said protein in the plant cell and not being capable of modulating the accumulation of the mRNA encoding said protein.

In one embodiment, the invention relates to the method as described above, said method allowing:
promote the development of a plant; or
slow down or prevent the development of a plant.

In particular, the invention relates to the method as described above, said method making it possible to promote the development of a plant. In particular, the invention relates to the method as described above, said method making it possible to slow down or prevent the development of a plant.

In one embodiment, the invention relates to the method as described above for increasing the accumulation of said protein in the plant cell. The presence of cPEP causes the amount of said protein in the treated plant cell to be greater than that in an untreated plant cell.

In one embodiment, the invention relates to the method as described above for decreasing (inhibiting) the accumulation of said protein in the plant cell. The presence of cPEP means that the quantity of the said protein in the treated plant cell is less than that in an untreated plant cell.

In one embodiment, the invention relates to the method as described above, wherein said cPEP is produced outside said plant cell before being introduced into said plant cell.

In one embodiment, the invention relates to the method as described above, wherein said cPEP is a synthetic peptide.

In one embodiment, the invention relates to the method as described above, wherein said cPEP is an isolated peptide.

In one embodiment, the invention relates to the method as described above, wherein said cPEP is a recombinant peptide.

In one embodiment, the invention relates to the method as described above, wherein said cPEP being a hydrophobic peptide or a hydrophilic peptide.

In one embodiment, the invention relates to the method as previously described, wherein said cPEP is introduced into said plant cell in the form of a nucleic acid encoding said cPEP. In particular, the invention relates to the method as described above, wherein said cPEP is introduced into said plant cell in the form of a nucleic acid encoding said cPEP and comprising the means for expressing it.

In one embodiment, the invention relates to the method as described above, wherein said protein is naturally present in said plant cell.

In one embodiment, the invention relates to the method as described above, wherein said protein is not naturally present in said plant cell. In particular, the invention relates to the method as described above, wherein said protein is encoded by a transgene artificially introduced into said plant cell. In particular, the invention relates to the method as described above, wherein said protein is encoded by a vector artificially introduced into said plant cell.

In one embodiment, the invention relates to the method as described above, wherein the accumulation of the said protein is determined using a technique selected from: Western blot, measurement of enzyme activity, mass spectrometry and translational fusion. In particular, the invention relates to the method as described above, wherein the accumulation of the said protein is determined using a Western blot.

In one embodiment, the invention relates to the method as described above, wherein said cPEP has a size from 4 to 41 amino acids, from 5 to 40 amino acids, from 7 to 20 amino acids or more particularly a size from 8 to 15 amino acids. In particular, said cPEP has a size from 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

In one embodiment, the invention relates to the method as described above, wherein said plant cell or said plant belongs to a plant species selected from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In particular, the invention relates to the method as described above, wherein said plant cell is an algal cell.

In one embodiment, the invention relates to the method as described above, wherein said protein is encoded by a gene selected from: Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2 h3, Cesa6, Cipk3, Cks1, Cob18, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexo1, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Skl, Sps1, Spt, Stn8, Tap46, Topp6, TubB6, TubB8, Uba1a, Vim3, Sgr1, Abi5, Hsp101, Rh10 and Wus.

In view of the above, it is understood that in another embodiment, the invention relates to the method as described above comprising a step for introducing:
a cPEP; or
of a nucleic acid encoding said cPEP and the means of expressing it, in said plant cell, the introduction of said cPEP resulting in a modulation of the quantity of said protein in said plant cell, said cPEP having a size from 4 to 70 amino acids, in particular from 4 to 41 amino acids, the amino acid sequence of which corresponds to the translation via the genetic code of a non-naturally translated fragment of a nucleic acid sequence naturally translated on an mRNA in a plant cell which corresponds to the open reading frame of said protein encoded by said mRNA, said non-naturally translated fragment lacking the initiation codon AUG encoding an initiator methionine and/or a STOP codon chosen from the codons: UAG, UGA and UAA, and being chosen either in the same reading frame as the open reading frame encoding said protein, or in a reading frame shifted by one or two nucleotides relative to the open reading frame encoding said protein, and said cPEP being capable of modulating the accumulation of said protein in the plant cell and not being capable of modulating the accumulation of the mRNA encoding said protein, said protein being encoded by a gene selected from: Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2 h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexo1, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Ski, Sps1, Spt, Stn8, Tap46, Topp6, TubB6, TubB8, Uba1a, Vim3, Sgr1, Abi5, Hsp101, Rh10 and Wus.

In one embodiment, the invention relates to the method as described above, wherein said protein is encoded by an ORF comprising a nucleic acid sequence having at least 80% identity, preferably at least 90% identity, with a sequence chosen from the sequences: SEQ ID NOs: 1 to 161 and 381 to 385.

In one embodiment, the invention relates to the method as described above, wherein said protein is encoded by an ORF comprising a nucleic acid sequence having at least 80% identity, preferably at least 90% identity, with a sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method as described above, wherein the said protein is encoded by a gene comprising a nucleic acid sequence having at least 80% identity, preferably at least 90% identity, with a sequence chosen from the sequences: SEQ ID NOs: 26 to 55 (Cpk3). In particular, the invention relates to the method as described above, wherein said protein is encoded by a gene comprising a nucleic acid sequence having at least 80% identity, preferably at least 90% identity, with a sequence chosen from the sequences: SEQ ID NOs: 61 to 79 (Dcl1). In particular, the invention relates to the method as described above, wherein said protein is encoded by a gene comprising a nucleic acid sequence having at least 80% identity, preferably at least 90% identity, with a sequence chosen from the sequences: SEQ ID NOs: 109 to 134 (Nsp1).

In one embodiment, the invention relates to the method as described above, wherein said protein is encoded by a gene comprising a nucleic acid sequence chosen from the sequences: SEQ ID NOs: 1 to 161 and 381 to 385. In one embodiment, the invention also relates to the method as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method as described above, wherein said protein is encoded by a gene comprising one selected from the sequences: SEQ ID NOs: 26 to 55 (Cpk3). In particular, the invention relates to the method as described above, wherein said protein is encoded by a gene comprising one selected from the sequences: SEQ ID NOs: 61 to 79 (Dcl1). In particular, the invention relates to the method as described above, wherein the said protein is encoded by a gene comprising one selected from the sequences: SEQ ID NOs: 109 to 134 (Nsp1).

In another embodiment, the invention relates to the method as described above, wherein said cPEP is chosen from the sequences: SEQ ID NOs: 162 to 206, 404 and 406 to 417.

In particular, the invention relates to the method as described above, wherein the said cPEP is chosen from the sequences: SEQ ID NO: 162, SEQ ID NO: 163 and SEQ ID NOs: 164 to 174. In particular, the invention relates to the method as described above, wherein the said cPEP is of sequence SEQ ID NO: 162. In particular, the invention relates to the method as previously described, wherein said cPEP is of sequence: SEQ ID NO: 163. In particular, the invention relates to the use of a cPEP as described above, wherein said cPEP is selected from the sequences: SEQ ID NOs: 164 to 174.

In another embodiment, the invention relates to the method as described above, wherein said protein is involved in at least one plant phenotype selected from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

In one embodiment, the invention relates to the method as described above, wherein the introduction of said cPEP leads to earlier bolting in said plant.

In one embodiment, the invention relates to the method as described above, wherein the introduction of said cPEP results in earlier flowering in said plant.

In one embodiment, the invention relates to the method as described above, wherein introduction of said cPEP results in an increase in stem size in said plant.

In one embodiment, the invention relates to the method as described above, wherein the introduction of said cPEP results in earlier stem growth in said plant.

The Inventors have unexpectedly found that it is possible to apply a cPEP directly to the plant, e.g. by using the composition of the invention (see above) comprising a cPEP, to modulate the accumulation of a target protein in the plant, which indicates that the cPEP is taken up by the plant.

Therefore, in one embodiment, the invention relates to the method as described above, wherein said cPEP is introduced into said plant:
by watering, by spraying or by adding a fertiliser, a potting soil, a culture substrate or a support in contact with the plant, the said cPEP being administered to the plant in particular in the form of a composition comprising from 10⁻⁹ M to 10⁻⁴ M of the said cPEP;
by watering, soaking, spraying or by adding a fertiliser, a potting soil, a growing substrate or a support in contact with the plant, the said cPEP being administered in particular to a seed or a seedling in the form of a composition comprising from 10⁻⁹ M to 10⁻⁴ M of the said cPEP; or
by means of a nucleic acid encoding said cPEP and comprising the means for expressing said cPEP, said nucleic acid being artificially introduced into the plant.

In one embodiment, the invention relates to the method as defined above, wherein said cPEP is artificially introduced externally into the plant, preferably by watering, spraying or by the addition of a fertiliser, potting soil, growing substrate or inert support.

In one embodiment, the invention relates to the method as defined above, wherein said cPEP is introduced by watering.

In one embodiment, the invention relates to the method as defined above, wherein said cPEP is introduced by spraying.

In one embodiment, the invention relates to the method as defined above, wherein said cPEP is introduced by the addition of a fertiliser.

In one embodiment, the invention relates to the method as defined above, wherein the plant is treated with a composition comprising from 10⁻⁹ M to 10⁻⁴ M of said cPEP, or comprising in particular 10⁻⁹ M, 10⁻⁸ M, 10⁻⁷ M, 10⁻⁶ M, 10⁻⁵ M or 10⁻⁴ M of said cPEP. Preferably, the compositions have a concentration of 10⁻⁸ M to 10⁻⁵ M for application to the plant by watering or spraying.

In addition, more or less concentrated compositions can be used to treat the plant with cPEP. For example, and without limitation, more concentrated compositions comprising from 10⁻¹ M to 10⁻³ M, or comprising in particular 10⁻² M of cPEP, can be used in the case where the cPEP artificially introduced externally is administered to the plant by spreading.

In another aspect, the above invention concerns a modified plant containing a cPEP, which “modified plant” corresponds to a plant into which a cPEP has been artificially introduced, in particular by watering, spraying or via a fertiliser.

In one embodiment, the invention relates to the modified plant comprising a cPEP introduced exogenously, said cPEP having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of a protein encoded by said mRNA, said cPEP being capable of modulating the accumulation of said protein in the plant cell and not being capable of modulating the accumulation of the mRNA encoding said protein.

In one embodiment, the invention relates to the modified plant as described above, wherein said fragment is devoid of:
the AUG initiation codon encoding a initiator methionine; or
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention therefore relates to the modified plant as described above, wherein said fragment comprises an AUG initiation codon encoding an initiator methionine and lacks a STOP codon chosen from the codons: UAG, UGA and UAA. The invention also relates to the modified plant as described above, wherein said fragment is devoid of an initiation codon AUG encoding an initiator methionine and comprises a STOP codon selected from codons: UAG, UGA and UAA.

In one embodiment, the invention relates to the modified plant as described above, wherein said fragment is devoid of:
the AUG initiation codon encoding a initiator methionine; and
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention relates to the modified plant as described above, wherein the translation of the mRNA fragment is carried out in the same reading frame as the open reading frame of the nucleic acid sequence naturally translated in said plant cell. In other words, the invention relates to the modified plant as described above, wherein the translation of the mRNA fragment is carried out in the reading frame determined by the initiation codon of the open reading frame of the said protein.

In one embodiment, the invention relates to the modified plant as described above, wherein the translation of the mRNA fragment is carried out in a reading frame different from the open reading frame of the nucleic acid sequence naturally translated in said plant cell. In particular, the invention relates to the modified plant as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the modified plant as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the modified plant as described above, wherein the translation of the mRNA fragment is carried out in a reading frame shifted by one (or two nucleotides at 5′) or two nucleotides at 3′ (or one nucleotide at 5′) with respect to the initiation codon of the open reading frame of the nucleic acid sequence naturally translated in said plant cell.

In one embodiment, the invention relates to a modified plant comprising an exogenously introduced cPEP, said cPEP having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of a protein encoded by said mRNA, said fragment having a size of 3n nucleotides, n being from 4 to 70, in particular n being from 4 to 41, and said nucleic acid sequence comprising two contiguous parts:
a part located within a nucleic acid sequence known to be non-coding (i.e. not naturally translated, e.g. 3′UTR or 5′UTR); and
a part located within a nucleic acid sequence considered to be coding (i.e. naturally translated, e.g. exon);
said cPEP being capable of modulating the accumulation of said protein in the plant cell and not being capable of modulating the accumulation of the mRNA encoding said protein.

In one embodiment, the invention relates to the modified plant as described above, said plant belonging to a plant species selected from: Alopecurus myosuroides, Amaranthus hypochondriacusAmaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In another aspect, the above invention relates to a transgenic plant comprising a nucleic acid encoding a cPEP and the means of expressing it, said cPEP having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of a protein encoded by said mRNA, said cPEP being capable of modulating the accumulation of said protein in the plant cell and not being capable of modulating the accumulation of the mRNA encoding said protein.

In one embodiment, the invention relates to the transgenic plant as described above, wherein said fragment is devoid of:
the AUG initiation codon encoding a initiator methionine; or
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention therefore relates to the transgenic plant as described above, wherein said fragment comprises an AUG initiation codon encoding an initiator methionine and lacks a STOP codon chosen from the codons: UAG, UGA and UAA. The invention also relates to the transgenic plant as described above, wherein the said fragment lacks an AUG initiation codon encoding an initiator methionine and comprises a STOP codon chosen from the codons: UAG, UGA and UAA.

In one embodiment, the invention relates to the transgenic plant as described above, wherein said fragment is devoid of:
the AUG initiation codon encoding a initiator methionine; and
a STOP codon chosen from: UAG, UGA and UAA,
   and said fragment being selected from:
either in the same open reading frame as that encoding the said protein;
or in an open reading frame shifted by one or two nucleotides relative to that encoding said protein.

In one embodiment, the invention relates to the transgenic plant as described above, wherein the translation of the mRNA fragment is carried out in the same reading frame as the open reading frame of the nucleic acid sequence naturally translated in said plant cell. In other words, the invention relates to the transgenic plant as described above, wherein the translation of the mRNA fragment is carried out in the reading frame determined by the initiation codon of the open reading frame of the said protein.

In one embodiment, the invention relates to the transgenic plant as described above, wherein the translation of the mRNA fragment is carried out in a reading frame different from the open reading frame of the nucleic acid sequence naturally translated in said plant cell. In particular, the invention relates to the transgenic plant as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the transgenic plant as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the transgenic plant as described above, wherein the translation of the mRNA fragment is carried out in a reading frame shifted by one (or two nucleotides at 5′) or two nucleotides at 3′ (or one nucleotide at 5′) with respect to the initiation codon of the open reading frame of the nucleic acid sequence naturally translated in the said plant cell.

In one embodiment, the invention relates to a transgenic plant comprising an exogenously introduced cPEP, said cPEP having a size from 4 to 70 amino acids, in particular the amino acid sequence of which corresponds to the translation via the genetic code of a fragment of a nucleic acid sequence naturally translated on an mRNA in a plant cell which corresponds to the open reading frame of a protein encoded by said mRNA, said fragment having a size of 3n nucleotides, n being from 4 to 70, in particular n being from 4 to 41, and said nucleic acid sequence comprising two contiguous parts:
a part located within a nucleic acid sequence known to be non-coding (i.e. not naturally translated, e.g. 3′UTR or 5′UTR); and
a part located within a nucleic acid sequence considered to be coding (i.e. naturally translated, e.g. exon);
said cPEP being capable of modulating the accumulation of said protein in the plant cell and not being capable of modulating the accumulation of the mRNA encoding said protein.

In one embodiment, the invention relates to the transgenic plant as defined above, wherein the sequence encoding said cPEP is shorter than the sequence of the mRNA encoding said protein.

In one embodiment, the invention relates to the transgenic plant as described above, said plant belonging to a plant species selected from: Alopecurus myosuroides, Amaranthus hypochondriacus, Amaranthus palmeri, Amaranthus tuberculatus, Arabidopsis halleri, Arabidopsis lyrata, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In one embodiment, the invention relates to the transgenic plant as described above, wherein expression of said cPEP is placed under the control of a strong promoter, preferably a constitutive strong promoter such as the 35S promoter.

In any event, it should be noted that the various aspects of Invention No. 1, like the various embodiments thereof, are interdependent. The latter may therefore be combined with one another to obtain aspects and/or preferred embodiments of Invention No. 1 not explicitly described. This also applies to all the definitions provided in this description, which apply to all aspects of Invention No. 1 and its embodiments.

Considering the second invention (altPEP), a first aspect thereof concerns a method for preparing and determining an altPEP, said altPEP:
having a size from 4 to 70 amino acids, in particular from 4 to 41 amino acids;
being capable of modulating the accumulation of a protein in a plant cell; and
not being capable of modulating the accumulation of the mRNA encoding said protein,
   said method comprising:
a. a step for determining the nucleic acid sequence of the messenger RNA (mRNA) encoding said protein;
b. a step for determining within this mRNA the nucleic acid sequence naturally translated in said plant cell and which corresponds to the open reading frame encoding said protein;
c. a step for determining, within this nucleic acid sequence naturally translated in said plant cell, a naturally translated fragment thereof, said fragment having a size of 3n nucleotides capable of being translated via the genetic code into a peptide, n being from 4 to 70, in particular n being from 4 to 41, and said fragment having a size smaller than that of the nucleic acid sequence naturally translated in said plant cell;
d. a step for producing said peptide; and
e. a comparison step:
   between the accumulation of said protein in a plant cell in the presence of said peptide and the accumulation of said protein in a plant cell of the same type in the absence of said peptide; and/or
   between the phenotype of a plant in the presence of said peptide and the phenotype of a plant of the same type in the absence of said peptide,
wherein:
   a difference in the amount of said protein in the presence of said peptide compared to the amount of said protein in the absence of said peptide; and/or
   a difference in the phenotype in the presence of said peptide compared with the phenotype in the absence of said peptide, indicates that said peptide is an altPEP capable of modulating the accumulation of said protein in a plant cell.

The present invention is based on the Inventors' unexpected observation that it is possible to specifically modulate the accumulation of a protein using a particular naturally produced peptide, the sequence of which corresponds to the translation of a fragment of the messenger RNA (mRNA) encoding said protein, said fragment being selected from the nucleic acid sequence naturally translated in said plant cell and which corresponds to the open reading frame encoding said protein.

In this invention, the term “altPEP” (alternative peptide) refers to a peptide capable of specifically modulating the accumulation of a protein once introduced into a plant cell.

According to the invention, an altPEP is naturally present in a plant cell. This means that the plant cell contains the altPEP information and the means to enable its expression (i.e. START codon and STOP codon).

An altPEP can therefore be present in a plant cell and the quantity of it can be modified by artificially adding it, in the form of a peptide or in the form of a nucleic acid encoding said peptide, to the plant cell.

The specificity of the altPEP with respect to a target protein (a target gene) is determined by its amino acid sequence. The sequence of an altPEP corresponds to the translation of a fragment of the mRNA encoding said protein, said fragment being selected from the nucleic acid sequence naturally translated in said plant cell and which corresponds to the open reading frame encoding said protein.

The peptide sequence of an altPEP can therefore be determined from a fragment of the mRNA encoding said protein.

In the invention, the mRNA fragment used to determine the altPEP sequence can be selected in the two reading frames other than the one encoding the protein whose accumulation is to be modulated existing on the mRNA sequence. In other words, a fragment can be selected in reading frames+2 or +3. On this point, it is possible that the other two reading frames (+2 and +3) contain the information of an altPEP, just as it is possible that only one of the two reading frames (+2 or +3) contains the information of an altPEP.

In the invention, the term “reading frame” refers to the grouping of nucleotides making up a nucleic acid sequence into consecutive triplets (or codons), which follow one another without interruption or overlap.

Generally speaking, altPEPs have a size from 4 to 70 amino acids, in particular from 4 to 41 amino acids, in particular a size from 5 to 40 amino acids, from 7 to 20 amino acids or more particularly a size from 8 to 15 amino acids. Consequently, the sequence of an altPEP corresponds to the translation of a fragment comprising from 4 to 41 triplets of nucleotides, in particular a fragment comprising from 5 to 40 triplets of nucleotides, from 7 to 20 triplets of nucleotides or more particularly a fragment comprising from 8 to 15 triplets of nucleotides.

In other words, the sequence of an altPEP corresponds to the translation into amino acids of a fragment of “3n” nucleotides of the mRNA of the target protein, n being from 4 to 70, in particular n being from 4 to 41, in particular from 5 to 40, from 7 to 20 or more particularly from 8 to 15.

For example, if n is equal to 5, the altPEP is 5 amino acids in size and corresponds to the translation of a fragment of 15 (=3×5) nucleotides. For example, if n is equal to 40, the altPEP is 40 amino acids in size and corresponds to the translation of a fragment of 120 (=3×40) nucleotides. For example, if n is equal to 70, the altPEP is 70 amino acids in size and corresponds to the translation of a fragment of 210 (=3×70) nucleotides. And so on. altPEPs are 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids, and correspond respectively to the translation of fragments of 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114, 117, 123, 126, 129, 132, 135, 138, 141, 144, 147, 150, 153, 156, 159, 162, 165, 168, 171, 174, 177, 180, 183, 186, 189, 192, 195, 198, 201, 204, 207 or 210 nucleotides.

In one embodiment, the invention relates to the method for preparing and determining an altPEP as described above, wherein said fragment has a size of 3n nucleotides, n being comprised:
from 4 to 41;
from 5 to 40;
from 7 to 20; or
from 8 to 15.

In one embodiment, the invention relates to the method for preparing and determining an altPEP as described above, wherein said peptide has a size selected from: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 and 70 amino acids.

In one embodiment, the invention relates to the method for preparing and determining an altPEP as described above, wherein said altPEP has a size smaller than that of said protein (i.e. the protein whose accumulation is modulated by the altPEP).

As indicated above, altPEPs have the ability to specifically modulate the accumulation of a protein without affecting the accumulation of the corresponding mRNA. In other words, the addition of an altPEP to a plant cell does not modify the quantity of mRNA used to express the protein that it has the capacity to regulate, but only the quantity of the said protein.

According to the invention, the term “protein” refers to a sequence of amino acids whose information is encoded by a gene present in the genome of a plant cell. By “gene” is meant, in particular, the nucleic acid sequence necessary for the synthesis of the said protein. A gene also includes more than the nucleotides encoding the amino acid sequence of the protein. For example, a gene includes the DNA sequences required to synthesise a pre-messenger (pre-mRNA), which is then matured by the cellular machinery into a messenger RNA (mRNA). This can then be translated into a protein via the ribosomes.

In view of the above, it is clear that pre-messenger RNA (pre-mRNA) has not undergone splicing and is likely to contain introns, whereas mature messenger RNA (mRNA) may have undergone splicing and contains only exons.

To prepare an altPEP capable of modulating the accumulation of a protein, it is necessary to translate a fragment of the mRNA encoding said protein, said fragment being chosen from the nucleic acid sequence naturally translated in said plant cell and which corresponds to the open reading frame encoding said protein, which comprises neither the 5′-UTR region nor the 3′-UTR region of the mRNA.

In the invention, “modulation” of the accumulation of a protein designates either an increase in the accumulation of said protein (i.e. an increase in the quantity of protein in the plant cell), or a decrease in the accumulation of said protein (i.e. a decrease in the quantity of protein in the plant cell). In other words, one embodiment of the invention relates to the method for preparing and determining an altPEP as described above, wherein said modulation of the accumulation of said protein induced by said altPEP is:
a reduction in the accumulation of the said protein; or
an increase in the accumulation of the said protein.

The increase and decrease in the accumulation of said protein can be measured and monitored using methods well known to those skilled in the art, such as coupling the protein to a marker using specific expression cassettes, or a Western blot.

In one embodiment, the invention relates to the method for preparing and determining an altPEP as described above, wherein in step e., the amount of protein in the presence of said peptide is greater than the amount of protein in the absence of said peptide. In other words, in the presence of an altPEP promoting increased protein accumulation, translation of the corresponding mRNA is increased, leading to greater production of the protein without altering the amount of said mRNA.

In another embodiment, the invention relates to the method for preparing and determining an altPEP as described above, wherein in step e., the amount of protein in the presence of said peptide is less than the amount of protein in the absence of said peptide. In other words, in the presence of an altPEP favouring a reduction in the accumulation of the protein, the translation of the corresponding mRNA is reduced (inhibited), which leads to a lower production of the protein without the quantity of said mRNA being modified.

In the invention, the fragment of the mRNA encoding said altPEP is located within the nucleic acid sequence naturally translated in said plant cell and which corresponds to the open reading frame encoding said protein, and said fragment as such can also be translated naturally in said plant cell. This is the case regardless of the reading frame used (i.e. the +2 and/or the +3). The existence of an altPEP within the said plant cell is therefore natural and originates either from the cellular machinery or from human action. To achieve this, it is possible either to artificially introduce said altPEP as such, or to introduce an expression cassette comprising the nucleic acid sequence encoding said altPEP and the means of expressing it in said plant cell.

In the invention, the nucleic acid sequence naturally translated in the said plant cell, which comprises a fragment carrying the information of an altPEP, is a region of the mRNA described as “coding”, i.e. it corresponds to a region of the mRNA which codes for all or part of the functional protein. This sequence therefore corresponds to the main open reading frame (i.e. the +1), which encodes the protein whose accumulation is to be modulated.

In the invention, the terms “open reading frame” and “ORF” are equivalent and can be used interchangeably. They correspond to a sequence of nucleotides (nucleic acids) in a DNA or RNA molecule that can potentially encode a peptide or protein: the said open reading frame begins with a START codon (the START codon generally encoding a methionine), followed by a series of codons (each codon encoding an amino acid), and ends with a STOP codon (the STOP codon not being translated).

The mRNA coding region therefore corresponds to the genetic sequence delimited by the START codon or initiation codon (most often encoding a methionine) at the 5′ end and by the STOP codon at the 3′ end. The mRNA coding region therefore does not include any intronic sequences that may be present in the sequence of a gene or pre-messenger RNA (pre-mRNA), or the 5′UTR and 3′UTR regions, as these are not translated and therefore do not code for part of the gene's functional protein.

In the invention, the sequence of an altPEP is determined by translating a fragment of the mRNA of the protein whose accumulation is to be modulated, said fragment being chosen from the (coding) nucleic acid sequence naturally translated in said plant cell and which corresponds to the open reading frame coding for said protein. Also, the same nucleic acid sequence naturally translated in said plant cell can give different altPEPs depending on the mRNA fragment chosen. Furthermore, this same mRNA fragment may also give different altPEPs depending on the reading frame used to translate it, i.e. depending on the grouping of the nucleotides of the sequence into consecutive triplets. As previously mentioned, a translation can be carried out in two different reading frames (the +2 and/or the +3), potentially leading to two different altPEPs.

According to the invention, the “+1” reading frame corresponds to the reading frame determined by the protein initiation codon, i.e. the START codon of the open reading frame naturally used for mRNA translation.

The “+2” and “+3” reading frames correspond to reading frames that are not or are rarely used naturally for mRNA translation. According to the invention, the “+2” reading frame corresponds to the reading frame shifted by one nucleotide at 3′ (or by two nucleotides at 5′) relative to the open reading frame naturally used for translation of the mRNA and the protein it encodes.

According to the invention, the “+3” reading frame corresponds to the reading frame shifted by two nucleotides at 3′ (or one nucleotide at 5′) with respect to the open reading frame naturally used for translation of the mRNA and the protein it encodes.

Generally, in the case of an mRNA fragment corresponding to a coding region and translated according to the +2 or +3 reading frame, the altPEPs obtained have a different sequence in comparison of the amino acid sequence of a fragment of the protein naturally encoded by the said mRNA.

In one embodiment, the invention relates to the method for preparing and determining an altPEP as described above, wherein said fragment comprises:
an initiation codon encoding an initiator methionine; and
a STOP codon chosen from: UAG, UGA and UAA, and wherein said fragment is chosen in a reading frame shifted by one or two nucleotides relative to the open reading frame encoding said protein.

In one embodiment, the invention relates to the method for preparing and determining an altPEP as described above, wherein the translation of the mRNA fragment is carried out in a reading frame different from the open reading frame of the nucleic acid sequence naturally translated in the said plant cell. In particular, the invention relates to the method for preparing and determining an altPEP as described above, wherein the translation of the mRNA fragment is carried out in a reading frame shifted by one nucleotide 3′ (or by two nucleotides 5′) with respect to the open reading frame of the nucleic acid sequence naturally translated in said plant cell. In particular, the invention also relates to the method for preparing and determining an altPEP as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the method for preparing and determining an altPEP as described above, wherein the translation of the mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in said plant cell.

In one embodiment, the invention relates to the method for preparing and determining an altPEP as described above, wherein said altPEP is a hydrophobic peptide or a hydrophilic peptide. In particular, the invention relates to the method for preparing and determining an altPEP as described above, wherein said altPEP is a hydrophobic peptide. By “hydrophobic peptide” is meant a peptide the amino acid sequence of which comprises more than 50% hydrophobic amino acids. More than 50%” means that the amino acid sequence comprises more than 55%, more than 60%, more than 65%, more than 70%, more than 75% or more than 80% hydrophobic amino acids. By “more than 50%” is also meant that the amino acid sequence comprises at least 51%, at least 56%, at least 61%, at least 66%, at least 71%, at least 76% or at least 81% hydrophobic amino acids. By “hydrophobic amino acids” is meant amino acids selected from: alanine (Ala/A), isoleucine (Ile/I), leucine (Leu/L), methionine (Met/M), phenylalanine (Phe/F), tryptophan (Trp/W), tyrosine (Tyr/Y) and valine (Val/V).

In particular, the invention also relates to the method for preparing and determining an altPEP as described above, wherein said altPEP is a hydrophilic peptide. By “hydrophilic peptide” is meant a peptide the amino acid sequence of which comprises more than 50% hydrophilic amino acids. More than 50%” means that the amino acid sequence comprises more than 55%, more than 60%, more than 65%, more than 70%, more than 75% or more than 80% hydrophilic amino acids. By “more than 50%” is also meant that the amino acid sequence comprises at least 51%, at least 56%, at least 61%, at least 66%, at least 71%, at least 76% or at least 81% hydrophilic amino acids. By “hydrophilic amino acids” is meant amino acids selected from: aspartic acid (Asp/D), glutamic acid (Glu/E), arginine (Arg/R), asparagine (Asn/N), glutamine (Gln/Q), histidine (His/H), lysine (Lys/K), serine (Ser/S) and threonine (Thr/T).

According to the invention, an altPEP can be produced by any type of means accessible to the person skilled in the art.

Without limitation, an altPEP can be produced either by synthesis or by recombinant expression in homologous or heterologous systems. The altPEP thus produced can then be introduced into a cell to modulate the accumulation of a target protein.

It is also possible to produce an altPEP directly in the plant cell containing the target protein, by artificially introducing a nucleic acid (such as an expression vector) encoding said altPEP.

In one embodiment, the invention relates to the method for preparing and determining an altPEP as described above, wherein, in step d., the said peptide is produced by peptide synthesis or by recombinant expression.

In one embodiment, the invention relates to the method for preparing and determining an altPEP as described above, wherein, in step d., said peptide is produced using a nucleic acid encoding said peptide introduced into a cell.

In one embodiment, the invention relates to the method for preparing and determining an altPEP as described above, wherein, in step e., the production of said peptide is carried out using a nucleic acid encoding said peptide introduced into said plant cell or into said plant.

In one embodiment, the invention relates to a method for preparing and determining an altPEP as described above, wherein, in step e., said peptide is brought into contact with said plant cell or in said plant.

In one embodiment, the invention relates to the method for preparing and determining an altPEP as described above, wherein, in step e., said peptide is present in said plant cell or in said plant following expression of a nucleic acid encoding said peptide in said plant cell or in said plant.

In view of the foregoing, it is understood that another embodiment of the invention relates to the method for preparing and determining an altPEP as described above, wherein, in step e., the presence of said peptide in said plant cell or in said plant results:
the introduction of a nucleic acid sequence encoding said peptide and comprising the means for expressing it; or
the introduction of an amino acid sequence corresponding to the said peptide.

An altPEP can be used to modulate the accumulation of a protein present naturally (i.e. endogenously) or not (i.e. exogenously) in said plant cell or in said plant.

A “protein naturally present in a plant cell or plant” is an endogenous protein encoded by a gene present in the genome of the plant cell or plant without the need for direct or indirect intervention by a human being.

A “protein which is not naturally present in a plant cell or in a plant” corresponds to an exogenous protein encoded by a nucleic acid sequence present on the genome of the plant cell or the plant which required the intervention of a human being and the use of means known to the person skilled in the art. Such a nucleic acid sequence may come from the same plant species or from another plant species.

In one embodiment, the invention relates to the method for preparing and determining an altPEP as described above, wherein said protein is of endogenous origin in said plant cells or plants used in step e.

In one embodiment, the invention relates to the method for preparing and determining an altPEP as described above, wherein said protein is of exogenous origin in said plant cells or said plants used in step e., said plant cells or said plants used in step e. then comprising a nucleic acid sequence allowing expression of said protein.

In one embodiment, the invention relates to the method for preparing and determining an altPEP as described above, wherein the accumulation of the said protein is determined using a technique chosen from: Western blot, measurement of enzymatic activity, mass spectrometry and translational fusion. In particular, the invention relates to the method for preparing and determining an altPEP as described above, wherein the accumulation of the said protein is determined using a Western blot.

Surprisingly, the Inventors have found that altPEPs can be used to modify the phenotypes of a plant that are visible on a macroscopic scale. It is therefore entirely possible to use altPEPs to confirm (or deny) that the peptide determined in steps a., b. and c., and possibly produced in step d., is an altPEP (or not). This is also made possible by the so-called phenotypic comparison alternative implemented in step e. For example, if the peptide determined on the mRNA of a protein involved in the size of the stem of a plant causes an increase, or a decrease, in the size of the stem of a plant treated with the latter compared with an untreated plant, this means that the said peptide is an altPEP capable of modulating the accumulation of the said protein in the size of the stem.

In the invention, the term “plant” refers generally to:
a set of plant cells organised in all or part of a plant, whatever its stage of development (including the plant in the form of a seed or young shoot);
one or more plant organs (e.g. leaves, roots, stems, flowers);
to one or more plant cells; or
a cluster of plant cells (e.g. a callus).

In the invention, the term “phenotype” designates, in a non-limiting manner, characters visible on a macroscopic scale such as the number of lateral roots, the number of leaves, the size of the stem, the duration of flowering and resistance to stress. In one embodiment, the invention therefore relates to the method for preparing and determining an altPEP as described above, wherein said protein is involved in at least one plant phenotype chosen from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

According to the invention, a protein is “involved in a phenotype” if a change in its accumulation is associated with a change in the said phenotype. In other words, a protein is involved in a phenotype if it is involved in the character(s) corresponding to the said phenotype.

In view of the foregoing, it is understood that an object of the invention is the method for preparing and determining an altPEP as described above, wherein the phenotype observed in step e. is chosen from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

In one embodiment, the invention relates to the method for preparing and determining an altPEP as described above, said altPEP:
having a size from 4 to 70 amino acids, in particular from 4 to 41 amino acids;
being capable of modulating the accumulation of a protein in a plant cell; and
not being capable of modulating the accumulation of the mRNA encoding said protein, and wherein the said plant cell (i.e. that wherein it is desired to modulate the accumulation of a protein) belongs to a plant species chosen from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In one embodiment, the invention relates to the method for preparing and determining an altPEP as described above, wherein said plant cells or said plants used in step e. belong to: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In one embodiment, the invention relates to the method for preparing and determining an altPEP as described above, wherein said plant cell (i.e. the one wherein it is desired to modulate the accumulation of a protein) is an algal cell.

In one embodiment, the invention relates to the method for preparing and determining an altPEP as described above, wherein said plant cells or said plants used in step e belong to an alga.

In one embodiment, the invention relates to the method for preparing and determining an altPEP as described above, wherein said protein is encoded by a gene selected from: Aae15 (Acyl-activating enzyme 15), Aae16 (AMP-dependent synthetase and ligase family protein), Abcg11 (White-brown complex-like protein), Abdcg34 (ABC transporter G family member 34), Acc1 (Acetyl-CoA Carboxylase), Agb1 (GTP binding protein beta 1), Als (Acetolactate synthase (chloroplastic)), Anac076 (NAC domain-containing protein 76), Apg9 (Autophagy 9), Arlb1 (GTP-binding protein 1), Arr1 (Two-component response regulator ARR1), Arr5 (Two-component response regulatorARR5), Arr6 (Two-component response regulatorARR6), At59 (Pectate lyase family protein), Bak1 (Brassinosteroid insensitive 1-associated receptor kinase 1), Bccp1 (Acetyl-CoA Carboxylase (chloroplastic) subunit 1), Bccp2 (Acetyl-CoA Carboxylase (chloroplastic) subunit 2), Bri1 (Brassinosteroid insensitive 1), Bzo2 h3 (bZIP transcription factor family protein), Cesa6 (Cellulose synthase A catalytic subunit 6), Cipk3 (CBL-interacting protein kinase 3), Cks1 (Cyclin-dependent kinases regulatory subunit 1), Cob/8 (COBRA-like protein 8 precursor), Coi1 (Coronatine-insensitive protein 1), Cpk3 (Calcium-dependent protein kinase 3), Crk34 (Cysteine-rich receptor-like protein kinase 34), Cyp705a18 (Cytochrome P450, family 705, subfamily A, polypeptide 18), Cyp71b26 (Cytochrome P450, family 71, subfamily B, polypeptide 26), Cyp78a8 (Cytochrome P450, family 78, subfamily A, polypeptide 8), Cyp97b3 (Cytochrome P450, family 97, subfamily B, polypeptide 3), Dcl1 (Endoribonuclease Dicer homolog 1), Dur3 (Urea-proton symporter DUR3), Ein2 (Ethylene-insensitive protein 2), Emb175 (Pentatricopeptide repeat-containing protein), Emb2726 (Elongation factor Ts family protein), Emb9 (Dihydrofolate synthetase), Epsps (5-enolpyruvylshikimate-3-phosphate (chloroplastic)), Fnr1 (Ferredoxin-NADP[+]-oxidoreductase 1), Fve (Transducin family protein/WD-40 repeat family protein), Ga2ox7 (Gibberellin 2-beta-dioxygenase 7), Gapc (Glyceraldehyde-3-phosphate dehydrogenase), Gcn2 (ABC transporter family protein), Gdi2 (Guanosine nucleotide diphosphate dissociation inhibitor 2), G1n2 (Glutamine synthetase (chloroplastic)), Gsl3 (Callose synthase 2), Hag5 (Histone acetyltransferase of the MYST family 2), Hda18 (Histone deacetylase 18), Hexo1 (Beta-hexosaminidase 1), Hppd (4-hydroxyphenyl-pyruvate-dioxygenase), Hsl1 (B3 domain-containing transcription repressor VAL2), Iaa31 (Indole-3-acetic acid inducible 31), Iqd28 (IQ-domain 28), Jac1 (J-domain protein required for chloroplast accumulation response 1), Jar1 (Jasmonoyl-L-amino acid synthetase), Kp1 (Kinesin-like protein 1), Lrx2 (Leucine-rich repeat/extensin 2), Mapkkk3 (Mitogen-activated protein kinase kinase kinase 3), Mapkkk5 (Mitogen-activated protein kinase kinase kinase 5), Mfp2 (Multifunctional protein 2), Mrb1 (Transmembrane protein, putative (DUF3537)), Nsp1 (Nodulation signaling pathway 1), Pds (Phytoene desaturase (chloroplastic)), Pen3 (Phosphatidylinositol 3,4,5-trisphosphate 3-phosphatase and protein-tyrosine-phosphatase), Phyb (Phytochrome B), Pif3 (Phytochrome interacting factor 3), Pizza (Brassinosteroid-related acyltransferase 1), Ppox1 (Protoporphyrinogen oxidase (chloroplastic) 1), Ppox2 (Protoporphyrinogen oxidase (chloroplastic) 2), Prp39 (Tetratricopeptide repeat (TPR)-like superfamily protein), PsbA (Photosystem II D1 protein), Pskr1 (Phytosulfokin receptor 1), Rd21 (Granulin repeat cysteine protease family protein), Ring1 (RING/U-box superfamily protein), Ros1 (DNA glycosylase/AP lyase ROS1), Rpt4a (26S proteasome regulatory subunit 10B homolog A), Sfr6 (Mediator of RNA polymerase II transcription subunit 16), Shr (Protein SHORT-ROOT), Shy2 (Auxin-responsive protein IAA3), Skl (EIN2-like protein, nramp transporter), Sps1 (Sucrose phosphate synthase 2F), Spt (Transcription factor SPATULA), Stn8 (Serine/threonine-protein kinase), Tap46 (PP2A regulatory subunit TAP46), Topp6 (Serine/threonine-protein phosphatase PP1 isozyme 7), TubB6 (Tubulin), TubB8 (Tubulin), Uba1a (RNA-binding (RRM/RBD/RNP motifs) family protein), Vim3 (E3 ubiquitin-protein ligase), Sgr1 (Magnesium dechelatase), Abi5 (Abscisic acid (ABA)-insensitive 5), Hsp101 (Heat shock protein 101), Rh10 (ATP-dependent RNA helicase) and Wus (WUSCHEL).

In particular, the invention relates to the method for preparing and determining an altPEP as described above, wherein said protein is encoded by a gene chosen from: Cpk3, Dcl and Nsp1. In particular, the invention relates to the method for preparing and determining an altPEP as described above, wherein said protein is encoded by the Cpk3 gene. In particular, the invention relates to the method for preparing and determining an altPEP as described above, wherein said protein is encoded by the Dcl gene. In particular, the invention relates to the method for preparing and determining an altPEP as described above, wherein said protein is encoded by the Nsp1 gene.

The genes Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2 h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexo1, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Skl, Sps1, Spt, Stn8, Tap46, Topp6, TubB6, TubB8, Uba1a, Vim3, Sgr1, Abi5, Hsp101, Rh10 and Wus refer to the proteins indicated in brackets. Of course, the invention also relates to homologous and/or similar genes that may bear different names. For example, in A. thaliana the Gsl3 gene encoding callose synthase 2 is also called Cals2.

In one embodiment, the invention relates to the method for preparing and determining an altPEP as described above, wherein said protein is encoded by an ORF comprising a nucleic acid sequence having at least 80% identity, preferably at least 90% identity, with a sequence chosen from the sequences: SEQ ID NO: 1 (ORF of the Aae15 protein, A. thaliana), SEQ ID NO: 2 (ORF of the Aae16 protein, A. thaliana), SEQ ID NO: 3 (ORF of the abcg11 protein, A. thaliana), SEQ ID NO: 4 (ORF of the Abdcg34 protein, A. thaliana), SEQ ID NO: 5 (ORF of the Acc1 protein, A. thaliana), SEQ ID NO: 6 (ORF of the Agb1 protein, A. thaliana), SEQ ID NO: 7 (ORF of the Als protein, A. thaliana), SEQ ID NO: 8 (ORF of the Anac076 protein, A. thaliana), SEQ ID NO: 9 (ORF of the Apg9 protein, A. thaliana), SEQ ID NO: 10 (ORF of the Arlb1 protein, A. thaliana), SEQ ID NO: 11 (ORF of Arr1 protein, A. thaliana), SEQ ID NO: 12 (ORF of Arr5 protein, A. thaliana), SEQ ID NO: 13 (ORF of Arr6 protein, A. thaliana), SEQ ID NO: 14 (ORF of At59 protein, A. thaliana), SEQ ID NO: 15 (ORF of Bak1 protein, A. thaliana), SEQ ID NO: 16 (ORF of the Bccp1 protein, A. thaliana), SEQ ID NO: 17 (ORF of the Bccp2 protein, A. thaliana), SEQ ID NO: 18 (ORF of the Bri1 protein, A. thaliana), SEQ ID NO: 19 (ORF of the Bzo2 h3 protein, A. thaliana), SEQ ID NO: 20 (ORF of the Cesa6 protein, A. thaliana), SEQ ID NO: 21 (ORF of the Cipk3 protein, A. thaliana), SEQ ID NO: 22 (ORF of the Cks1 protein, A. thaliana), SEQ ID NO: 23 (ORF of the Cobl8 protein, A. thaliana), SEQ ID NO: 24 (ORF of Coi1 protein, A. thaliana), SEQ ID NO: 25 (ORF of Coi1 protein, A. thaliana), SEQ ID NO: 26 (ORF of Cpk3 protein, A. thaliana), SEQ ID NO: 27 (ORF of Cpk3 protein, A. hypochondriacus), SEQ ID NO: 28 (ORF of Cpk3 protein, B. distachyon), SEQ ID NO: 29 (ORF of Cpk3 protein, B. distachyon), SEQ ID NO: 30 (ORF of Cpk3 protein, G. max), SEQ ID NO: 31 (ORF of Cpk3 protein, G. max), SEQ ID NO: 32 (ORF of Cpk3 protein, G. max), SEQ ID NO: 33 (ORF of Cpk3 protein, G. max), SEQ ID NO: 34 (ORF of Cpk3 protein, O. sativa), SEQ ID NO: 35 (ORF of Cpk3 protein, O. sativa), SEQ ID NO: 36 (ORF of Cpk3 protein, S. lycopersicum), SEQ ID NO: 37 (ORF of Cpk3 protein, Z. mays), SEQ ID NO: 38 (ORF of Cpk3 protein, Z. mays), SEQ ID NO: 39 (ORF of Cpk3 protein, Z. mays), SEQ ID NO: 40 (ORF of Cpk3 protein, B. rapa), SEQ ID NO: 41 (ORF of Cpk3 protein, B. rapa), SEQ ID NO: 42 (ORF of Cpk3 protein, H. vulgare), SEQ ID NO: 43 (ORF of Cpk3 protein, H. vulgare), SEQ ID NO: 44 (ORF of Cpk3 protein, S. tuberosum), SEQ ID NO: 45 (ORF of Cpk3 protein, A. palmeri), SEQ ID NO: 46 (ORF of Cpk3 protein, M. truncatula), SEQ ID NO: 47 (ORF of Cpk3 protein, M. truncatula), SEQ ID NO: 48 (ORF of Cpk3 protein, T. aestivum), SEQ ID NO: 49 (ORF of Cpk3 protein, T. aestivum), SEQ ID NO: 50 (ORF of Cpk3 protein, T. aestivum), SEQ ID NO: 51 (ORF of Cpk3 protein, T. aestivum), SEQ ID NO: 52 (ORF of Cpk3 protein, L. perenne), SEQ ID NO: 53 (ORF of Cpk3 protein, L. perenne), SEQ ID NO: 54 (ORF of Cpk3 protein, L. perenne), SEQ ID NO: 55 (ORF of protein Cpk3, L. perenne), SEQ ID NO: 56 (ORF of protein Crk34, A. thaliana), SEQ ID NO: 57 (ORF of protein Cyp705a18, A. thaliana), SEQ ID NO: 58 (ORF of protein Cyp71b26, A. thaliana), SEQ ID NO: 59 (ORF of protein Cyp78a8, A. thaliana), SEQ ID NO: 60 (ORF of Cyp97b3 protein, A. thaliana), SEQ ID NO: 61 (ORF of Ddll protein, A. thaliana), SEQ ID NO: 62 (ORF of Ddll protein, A. thaliana), SEQ ID NO: 63 (ORF of Ddll protein, A. hypochondriacus), SEQ ID NO: 64 (ORF of Ddll protein, B. distachyon), SEQ ID NO: 65 (ORF of Ddll protein, G. max), SEQ ID NO: 66 (ORF of Ddll protein, G. max), SEQ ID NO: 67 (ORF of Ddll protein, O. sativa), SEQ ID NO: 68 (ORF of Ddll protein, S. lycopersicum), SEQ ID NO: 69 (ORF of Ddll protein, Z. mays), SEQ ID NO: 70 (ORF of Ddll protein, B. rapa), SEQ ID NO: 71 (ORF of Ddll protein, H. vulgare), SEQ ID NO: 72 (ORF of Ddll protein, S. tuberosum), SEQ ID NO: 73 (ORF of Ddll protein, M. truncatula), SEQ ID NO: 74 (ORF of Ddll protein, T. aestivum), SEQ ID NO: 75 (ORF of Ddll protein, T. aestivum), SEQ ID NO: 76 (ORF of the Doll protein, T. aestivum), SEQ ID NO: 77 (ORF of the Doll protein, T. aestivum), SEQ ID NO: 78 (ORF of the Doll protein, L. perenne), SEQ ID NO: 79 (ORF of the Doll protein, L. perenne), SEQ ID NO: 80 (ORF of the Dur3 protein, A. thaliana), SEQ ID NO: 81 (ORF of the Ein2 protein, A. thaliana), SEQ ID NO: 82 (ORF of Emb175 protein, A. thaliana), SEQ ID NO: 83 (ORF of Emb2726 protein, A. thaliana), SEQ ID NO: 84 (ORF of Emb9 protein, A. thaliana), SEQ ID NO: 85 (ORF of Epsps protein, A. thaliana), SEQ ID NO: 86 (ORF of Fnr1 protein, A. thaliana), SEQ ID NO: 87 (ORF of the Fve protein, A. thaliana), SEQ ID NO: 88 (ORF of the Ga2ox7 protein, A. thaliana), SEQ ID NO: 89 (ORF of the Gapc protein, N. benthamiana), SEQ ID NO: 90 (ORF of the Gcn2 protein, A. thaliana), SEQ ID NO: 91 (ORF of the Gdi2 protein, A. thaliana), SEQ ID NO: 92 (ORF of GIn2 protein, A. thaliana), SEQ ID NO: 93 (ORF of Gsl3 protein, A. thaliana), SEQ ID NO: 94 (ORF of Hag5 protein, A. thaliana), SEQ ID NO: 95 (ORF of the Hda18 protein, A. thaliana), SEQ ID NO: 96 (ORF of the Hexo1 protein, A. thaliana), SEQ ID NO: 97 (ORF of the Hppd protein, A. thaliana), SEQ ID NO: 98 (ORF of the Hsl1 protein, A. thaliana), SEQ ID NO: 99 (ORF of the Iaa31 protein, A. thaliana), SEQ ID NO: 100 (ORF of the Iqd28 protein, A. thaliana), SEQ ID NO: 101 (ORF of the Jac1 protein, A. thaliana), SEQ ID NO: 102 (ORF of the Jar1 protein, A. thaliana), SEQ ID NO: 103 (ORF of the Kp1 protein, A. thaliana), SEQ ID NO: 104 (ORF of the Lrx2 protein, A. thaliana), SEQ ID NO: 105 (ORF of the Mapkkk3 protein, A. thaliana), SEQ ID NO: 106 (ORF of the Mapkkk5 protein, A. thaliana), SEQ ID NO: 107 (ORF of the Mfp2 protein, A. thaliana), SEQ ID NO: 108 (ORF of the Mrb1 protein, A. thaliana), SEQ ID NO: 109 (ORF of the Nsp1 protein, M. truncatula), SEQ ID NO: 110 (ORF of the Nsp1 protein, A. thaliana), SEQ ID NO: 111 (ORF of the Nsp1 protein, B. distachyon), SEQ ID NO: 112 (ORF of the Nsp1 protein, G. max), SEQ ID NO: 113 (ORF of the Nsp1 protein, G. max), SEQ ID NO: 114 (ORF of the Nsp1 protein, O. sativa), SEQ ID NO: 115 (ORF of the Nsp1 protein, S. lycopersicum), SEQ ID NO: 116 (ORF of the Nsp1 protein, S. lycopersicum), SEQ ID NO: 117 (ORF of the Nsp1 protein, Z. mays), SEQ ID NO: 118 (ORF of the Nsp1 protein, Z. mays), SEQ ID NO: 119 (ORF of Nsp1 protein, Z. mays), SEQ ID NO: 120 (ORF of Nsp1 protein, Z. mays), SEQ ID NO: 121 (ORF of Nsp1 protein, B. rapa), SEQ ID NO: 122 (ORF of the Nsp1 protein, H. vulgare), SEQ ID NO: 123 (ORF of the Nsp1 protein, H. vulgare), SEQ ID NO: 124 (ORF of the Nsp1 protein, H. vulgare), SEQ ID NO: 125 (ORF of the Nsp1 protein, H. vulgare), SEQ ID NO: 126 (ORF of the Nsp1 protein, H. vulgare), SEQ ID NO: 127 (ORF of the Nsp1 protein, H. vulgare), SEQ ID NO: 128 (ORF of the Nsp1 protein, H. vulgare), SEQ ID NO: 129 (ORF of the Nsp1 protein, S. tuberosum), SEQ ID NO: 130 (ORF of the Nsp1 protein, S. tuberosum), SEQ ID NO: 131 (ORF of the Nsp1 protein, T. aestivum), SEQ ID NO: 132 (ORF of the Nsp1 protein, T. aestivum), SEQ ID NO: 133 (ORF of the Nsp1 protein, L. perenne), SEQ ID NO: 134 (ORF of the Nsp1 protein, L. perenne), SEQ ID NO: 135 (ORF of the Pds protein, A. thaliana), SEQ ID NO: 136 (ORF of the Pen3 protein, A. thaliana), SEQ ID NO: 137 (ORF of the Phyb protein, A. thaliana), SEQ ID NO: 138 (ORF of the Pif3 protein, A. thaliana), SEQ ID NO: 139 (ORF of the Pizza protein, A. thaliana), SEQ ID NO: 140 (ORF of the Ppox1 protein, A. thaliana), SEQ ID NO: 141 (ORF of the Ppox2 protein, A. thaliana), SEQ ID NO: 142 (ORF of the Prp39 protein, A. thaliana), SEQ ID NO: 143 (ORF of the PsbA protein, A. thaliana), SEQ ID NO: 144 (ORF of the Pskr1 protein, A. thaliana), SEQ ID NO: 145 (ORF of the Rd21 protein, A. thaliana), SEQ ID NO: 146 (ORF of the Ring1 protein, A. thaliana), SEQ ID NO: 147 (ORF of the Ros1 protein, A. thaliana), SEQ ID NO: 148 (ORF of the Rpt4a protein, A. thaliana), SEQ ID NO: 149 (ORF of the Sfr6 protein, A. thaliana), SEQ ID NO: 150 (ORF of the Shr protein, A. thaliana), SEQ ID NO: 151 (ORF of the Shy2 protein, A. thaliana), SEQ ID NO: 152 (ORF of the Skl protein, M. truncatula), SEQ ID NO: 153 (ORF of the Sps1 protein, A. thaliana), SEQ ID NO: 154 (ORF of the Spt protein, A. thaliana), SEQ ID NO: 155 (ORF of the Stn8 protein, A. thaliana), SEQ ID NO: 156 (ORF of the Tap46 protein, A. thaliana), SEQ ID NO: 157 (ORF of the Topp6 protein, A. thaliana), SEQ ID NO: 158 (ORF of the TubB6 protein, A. thaliana), SEQ ID NO: 159 (ORF of the TubB8 protein, A. thaliana), SEQ ID NO: 160 (ORF of the Uba1a protein, A. thaliana), SEQ ID NO: 161 (ORF of the Vim3 protein, A. thaliana), SEQ ID NO: 381 (ORF of the Sgr1 protein, A. thaliana), SEQ ID NO: 382 (ORF of the Abi5 protein, A. thaliana), SEQ ID NO: 383 (ORF of the Hsp101 protein, A. thaliana), SEQ ID NO: 384 (ORF of the Rh10 protein, M. truncatula) and SEQ ID NO: 385 (ORF of the Wus protein, A. thaliana).

In one embodiment, the invention relates to the method for preparing and determining an altPEP as described above, wherein said protein is encoded by an ORF comprising a nucleic acid sequence having at least 80% identity, preferably at least 90% identity, with a sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method for preparing and determining an altPEP as described above, wherein the said protein is encoded by a gene comprising a nucleic acid sequence having at least 80% identity, preferably at least 90% identity, with a sequence chosen from the sequences: SEQ ID NOs: 26 to 55 (Cpk3). In particular, the invention relates to the method for preparing and determining an altPEP as described above, wherein said protein is encoded by a gene comprising a nucleic acid sequence having at least 80% identity, preferably at least 90% identity, with a sequence chosen from the sequences: SEQ ID NOs: 61 to 79 (Dcl1). In particular, the invention relates to the method for preparing and determining an altPEP as described above, wherein said protein is encoded by a gene comprising a nucleic acid sequence having at least 80% identity, preferably at least 90% identity, with a sequence chosen from the sequences: SEQ ID NOs: 109 to 134 (Nsp1).

“Percentage identity” between two nucleic acid (or amino acid) sequences refers to the percentage of identical nucleotides (or amino acid residues) between the two sequences to be compared, obtained after the best alignment. This percentage is purely statistical and the differences between the two sequences are randomly distributed over the entire length of the sequences. The best alignment (or optimal alignment) is the alignment for which the percentage of identity between the two sequences to be compared, as calculated below, is the highest. Sequence comparisons between two nucleic acid (or amino acid) sequences are traditionally performed by comparing these sequences after they have been optimally aligned, said comparison being performed by segment or by comparison window to identify and compare local regions of sequence similarity. The optimal alignment of sequences for comparison can be carried out manually or using algorithms and software available to those skilled in the art, for example, the BLAST platform or the MatGat program (Campanella, Bitincka and Smalley, 2003).

The percentage of identity between two sequences is determined by comparing these two optimally aligned sequences by comparison window wherein the region of the sequence to be compared may comprise additions or deletions relative to the reference sequence for optimal alignment between these two sequences. The percentage identity is calculated by determining the number of identical positions for which the nucleotide (or amino acid) is identical between the two sequences, dividing this number of identical positions by the total number of positions in the comparison window and multiplying the result obtained by 100.

Within the meaning of the invention, it is understood that sequences showing “at least 80% identity” with a reference sequence may in particular show at least 80%, 81%, 82%, 83%, 84%, 85%, 86%, 87%, 88%, 89%, 90%, 91%, 92%, 93%, 94%, 95%, 96%, 97%, 98%, 99% or 100% identity with said reference sequence.

In one embodiment, the invention relates to the method for preparing and determining an altPEP as described above, wherein the said protein is encoded by a gene comprising a nucleic acid sequence chosen from the sequences: SEQ ID NOs: 1 to 161 and 381 to 385. In one embodiment, the invention also relates to the method for preparing and determining an altPEP as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method for preparing and determining an altPEP as described above, wherein said protein is encoded by a gene comprising one selected from the sequences: SEQ ID NOs: 26 to 55 (Cpk3). In particular, the invention relates to the method for preparing and determining an altPEP as described above, wherein said protein is encoded by a gene comprising one selected from the sequences: SEQ ID NOs: 61 to 79 (Dcl1). In particular, the invention relates to the method for preparing and determining an altPEP as described above, wherein said protein is encoded by a gene comprising one selected from the sequences: SEQ ID NOs: 109 to 134 (Nsp1).

In another embodiment, the invention relates to the method for preparing and determining an altPEP as described above, wherein the sequence of said peptide is chosen from the sequences: SEQ ID NO: 224 (AtDCL1alt18), SEQ ID NO: 225 (CrDCL1alt18), SEQ ID NO: 226 (EsDCL1alt18), SEQ ID NO: 227 (AIDCL1 alt19), SEQ ID NO: 228 (CsDCL1alt19), SEQ ID NO: 229 (RsDCL1alt15), SEQ ID NO: 230 (BnDCL1alt14), SEQ ID NO: 231 (BoDCL1alt15), SEQ ID NO: 232 (BRDCL1alt17), SEQ ID NO: 233 (BsEIN2alt1), SEQ ID NO: 234 (CgEIN2alt1), SEQ ID NO: 235 (CrEIN2alt1), SEQ ID NO: 236 (EsEIN2alt1), SEQ ID NO: 237 (ThEIN2alt1), SEQ ID NO: 238 (BrEIN2alt1), SEQ ID NO: 239 (BoEIN2alt1), SEQ ID NO: 240 (BnEIN2alt1), SEQ ID NO: 241 (AtEIN2alt1), SEQ ID NO: 242 (AIEIN2alt1), SEQ ID NO: 243 (AtAP2alt1), SEQ ID NO: 244 (AIAP2alt1), SEQ ID NO: 245 (BoAP2alt1), SEQ ID NO: 246 (CgAP2alt1), SEQ ID NO: 247 (CrAP2alt1), SEQ ID NO: 248 (BrAP2alt1), SEQ ID NO: 249 (BsAP2alt1), SEQ ID NO: 250 (EsAP2alt1), SEQ ID NO: 251 (ThAP2alt1), SEQ ID NO: 252 (altPEP_DCL1), SEQ ID NO: 253 (altPEP_CYP78A8), SEQ ID NO: 254 (altPEP_PRP39), SEQ ID NO: 255 (altPEP_PIF3), SEQ ID NO: 256 (altPEP_PIF3), SEQ ID NO: 257 (altPEP_IQD28), SEQ ID NO: 258 (altPEP_AT59), SEQ ID NO: 259 (altPEP_AT59), SEQ ID NO: 260 (altPEP_ABCG11), SEQ ID NO: 261 (altPEP_ABCG11), SEQ ID NO: 262 (altPEP_ABCG11), SEQ ID NO: 263 (altPEP_ABCG11), SEQ ID NO: 264 (altPEP_ABCG11), SEQ ID NO: 265 (altPEP_ABCG11), SEQ ID NO: 266 (altPEP_ABCG11), SEQ ID NO: 267 (altPEP_RD21), SEQ ID NO: 268 (altPEP_RD21), SEQ ID NO: 269 (altPEP_RD21), SEQ ID NO: 270 (altPEP_RD21), SEQ ID NO: 271 (altPEP_LRX2), SEQ ID NO: 272 (altPEP_LRX2), SEQ ID NO: 273 (altPEP_LRX2), SEQ ID NO: 274 (altPEP_JAC1), SEQ ID NO: 275 (altPEP_JAC1), SEQ ID NO: 276 (altPEP_JAC1), SEQ ID NO: 277 (altPEP_PSKR1), SEQ ID NO: 278 (altPEP_PSKR1), SEQ ID NO: 279 (altPEP_PSKR1), SEQ ID NO: 280 (altPEP_PSKR1), SEQ ID NO: 281 (altPEP_PSKR1), SEQ ID NO: 282 (altPEP_PSKR1), SEQ ID NO: 283 (altPEP_FVE), SEQ ID NO: 284 (altPEP_FVE), SEQ ID NO: 285 (altPEP_UBA1A), SEQ ID NO: 286 (altPEP_CIPK3), SEQ ID NO: 287 (altPEP_CIPK3), SEQ ID NO: 288 (altPEP_APG9), SEQ ID NO: 289 (altPEP_APG9), SEQ ID NO: 290 (altPEP_COI1), SEQ ID NO: 291 (altPEP_COI1), SEQ ID NO: 292 (altPEP_COI1), SEQ ID NO: 293 (altPEP_COI1), SEQ ID NO: 294 (altPEP_COI1), SEQ ID NO: 295 (altPEP_COI1), SEQ ID NO: 296 (altPEP_COI1), SEQ ID NO: 297 (altPEP_COI1), SEQ ID NO: 298 (altPEP_COI1), SEQ ID NO: 299 (altPEP_MFP2), SEQ ID NO: 300 (altPEP_MFP2), SEQ ID NO: 301 (altPEP_MFP2), SEQ ID NO: 302 (altPEP_COBL8), SEQ ID NO: 303 (altPEP_IAA31), SEQ ID NO: 304 (altPEP_CYP705A18), SEQ ID NO: 305 (altPEP_AAE16), SEQ ID NO: 306 (altPEP_AAE16), SEQ ID NO: 307 (altPEP_CYP71B26), SEQ ID NO: 308 (altPEP_CYP71B26), SEQ ID NO: 309 (altPEP_CYP71B26), SEQ ID NO: 310 (altPEP_CYP71B26), SEQ ID NO: 311 (altPEP_CYP71B26), SEQ ID NO: 312 (altPEP_CYP71B26), SEQ ID NO: 313 (altPEP_KP1), SEQ ID NO: 314 (altPEP_KP1), SEQ ID NO: 315 (altPEP_PEN3), SEQ ID NO: 316 (altPEP_PEN3), SEQ ID NO: 317 (altPEP_HEXO1), SEQ ID NO: 318 (altPEP_GD12), SEQ ID NO: 319 (altPEP_GD12), SEQ ID NO: 320 (altPEP_GD12), SEQ ID NO: 321 (altPEP_GD12), SEQ ID NO: 322 (altPEP_GD12), SEQ ID NO: 323 (altPEP_SFR6), SEQ ID NO: 324 (altPEP_CRK34), SEQ ID NO: 325 (altPEP_AAE15), SEQ ID NO: 326 (altPEP_CYP97B3), SEQ ID NO: 327 (altPEP_CYP97B3), SEQ ID NO: 328 (altPEP_CYP97B3), SEQ ID NO: 329 (altPEP_CYP97B3), SEQ ID NO: 330 (altPEP_AMB2726), SEQ ID NO: 331 (altPEP_HSL1), SEQ ID NO: 332 (altPEP_HSL1), SEQ ID NO: 333 (altPEP_HSL1), SEQ ID NO: 334 (altPEP_ANAC076), SEQ ID NO: 335 (altPEP_STN8), SEQ ID NO: 336 (altPEP_EMB175), SEQ ID NO: 337 (altPEP_EMB175), SEQ ID NO: 338 (altPEP_EMB175), SEQ ID NO: 339 (altPEP_EMB175), SEQ ID NO: 340 (altPEP_EMB175), SEQ ID NO: 341 (altPEP_GCN2), SEQ ID NO: 342 (altPEP_SPS1), SEQ ID NO: 343 (altPEP_SPS1), SEQ ID NO: 344 (altPEP_SPS1), SEQ ID NO: 345 (altPEP_SPS1), SEQ ID NO: 346 (altPEP_BZO2H3), SEQ ID NO: 347 (altPEP_VIM3), SEQ ID NO: 348 (altPEP_EMB9), SEQ ID NO: 349 (altPEP_EMB9), SEQ ID NO: 350 (altPEP_RPT4A), SEQ ID NO: 351 (altPEP_TOPP6), SEQ ID NO: 352 (altPEP_DUR3), SEQ ID NO: 353 (altPEP_DUR3), SEQ ID NO: 354 (altPEP_DUR3), SEQ ID NO: 355 (altPEP_ARLB1), SEQ ID NO: 356 (altPEP_ARLB1), SEQ ID NO: 357 (altPEP_HDA18), SEQ ID NO: 358 (altPEP_HDA18), SEQ ID NO: 359 (altPEP_HDA18), SEQ ID NO: 360 (altPEP_CESA6), SEQ ID NO: 361 (altPEP_FNR1), SEQ ID NO: 362 (altPEP_FNR1), SEQ ID NO: 363 (altPEP_FNR1), SEQ ID NO: 364 (EIN2alt1), SEQ ID NO: 365 (EIN2alt2) and SEQ ID NO: 366 (EIN2alt3).

In a second aspect, the object of the above invention is an altPEP as obtained by implementing the method as described above. According to this same aspect, the invention also relates to an isolated altPEP, from 4 to 70 amino acids, in particular from 4 to 41 amino acids, the amino acid sequence of which corresponds to the translation via the genetic code of a naturally translated fragment of a nucleic acid sequence naturally translated on an mRNA in a plant cell which corresponds to the open reading frame of a protein encoded by the said mRNA, said altPEP being capable of modulating the accumulation of said protein in the plant cell and not being capable of modulating the accumulation of the mRNA encoding said protein.

In one embodiment, the invention relates to the isolated altPEP as previously described, said fragment having a size of 3n nucleotides, n being comprised:
from 4 to 41;
from 5 to 40;
from 7 to 20; or
from 8 to 15.

In other words, the invention relates to the isolated altPEP as described above, said isolated altPEP comprising 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

In particular, the invention relates to the isolated altPEP as described above, said isolated altPEP comprising from 4 to 41 amino acids. In particular, the invention relates to the isolated altPEP as described above, said isolated altPEP comprising from 5 to 40 amino acids. In particular, the invention relates to the isolated altPEP as described above, said isolated altPEP comprising from 7 to 20 amino acids. In particular, the invention also relates to the isolated altPEP as described above, said isolated altPEP comprising from 8 to 15 amino acids.

In one embodiment, the invention relates to the isolated altPEP as described above, wherein the size of said isolated altPEP is smaller than that of said protein.

In one embodiment, the invention relates to the isolated altPEP as described above, said fragment comprising:
an initiation codon encoding an initiator methionine; and
a STOP codon chosen from: UAG, UGA and UAA, and said fragment being chosen in an open reading frame shifted by one or two nucleotides relative to that encoding said protein.

In one embodiment, the invention relates to the isolated altPEP as described above, wherein the translation of the mRNA fragment is carried out in a reading frame different from the open reading frame of the nucleic acid sequence naturally translated in said plant cell. In particular, the invention relates to the isolated altPEP as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the isolated altPEP as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the isolated altPEP as described above, wherein the translation of the mRNA fragment is carried out in a reading frame shifted by one (or two nucleotides at 5′) or two nucleotides at 3′ (or one nucleotide at 5′) with respect to the initiation codon of the open reading frame of the nucleic acid sequence naturally translated in said plant cell.

In one embodiment, the invention relates to the isolated altPEP as described above, wherein said isolated altPEP is capable of increasing the accumulation of said protein in said plant cell.

In one embodiment, the invention relates to the isolated altPEP as described above, wherein said isolated altPEP is capable of decreasing the accumulation of said protein in said plant cell.

In one embodiment, the invention relates to the isolated altPEP as described above, wherein said isolated altPEP is a synthetic peptide.

In one embodiment, the invention relates to altPEP as described above, wherein said isolated altPEP is a recombinant peptide.

In one embodiment, the invention relates to the isolated altPEP as described above, said isolated altPEP being a hydrophobic peptide or a hydrophilic peptide.

In one embodiment, the invention relates to the isolated altPEP as described above, wherein said protein is naturally present in said plant cell.

In one embodiment, the invention relates to the isolated altPEP as described above, wherein said protein is not naturally present in said plant cell. In particular, the invention relates to the isolated altPEP as described above, wherein said protein is encoded by a transgene artificially introduced into said plant cell. In particular, the invention relates to the isolated altPEP as described above, wherein said protein is encoded by a vector artificially introduced into said plant cell.

In one embodiment, the invention relates to the isolated altPEP as described above, wherein said plant cell (i.e. the one wherein it is desired to modulate the accumulation of a protein) belongs to a plant species chosen from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In one embodiment, the invention relates to the isolated altPEP as described above, wherein said plant cell is an algal cell.

In one embodiment, the invention relates to the isolated altPEP as described above, wherein said protein is encoded by a gene selected from: Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2 h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, G1n2, Gsl3, Hag5, Hda18, Hexo1, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Ski, Sps1, Spt, Stn8, Tap46, Topp6, TubB6, TubB8, Uba1a, Vim3, Sgr1, Abi5, Hsp101, Rh10 and Wus.

In particular, the invention relates to the isolated altPEP as described above, wherein said protein is encoded by a gene selected from the genes: Cpk3, Dcl1 and Nsp1. In particular, the invention relates to the isolated altPEP as described above, wherein the said protein is encoded by the Cpk3 gene. In particular, the invention relates to the isolated altPEP as described above, wherein said protein is encoded by the Dcl1 gene. In particular, the invention relates to the isolated altPEP as described above, wherein said protein is encoded by the Nsp1 gene.

In one embodiment, the invention relates to the isolated altPEP as described above, wherein said protein is encoded by a gene comprising a nucleic acid sequence having at least 80% identity, preferably at least 90% identity, with a sequence selected from the sequences: SEQ ID NOs: 1 to 161 and 381 to 385.

In particular, the invention relates to the isolated altPEP as described above, wherein the said protein is encoded by a gene comprising a nucleic acid sequence having at least 80% identity, preferably at least 90% identity, with a sequence chosen from the sequences: SEQ ID NOs: 26 to 55 (Cpk3), SEQ ID NOs: 61 to 79 (Dcl1) and SEQ ID NOs: 109 to 134 (Nsp1). In particular, the invention also relates to the isolated altPEP as described above, wherein said protein is encoded by a gene comprising a nucleic acid sequence having at least 80% identity, preferably at least 90% identity, with a sequence selected from the sequences: SEQ ID NOs: 26 to 55 (Cpk3). In particular, the invention also relates to the isolated altPEP as described above, wherein said protein is encoded by a gene comprising a nucleic acid sequence having at least 80% identity, preferably at least 90% identity, with a sequence selected from the sequences: SEQ ID NOs: 61 to 79 (Dcl1). In particular, the invention also relates to the isolated altPEP as described above, wherein said protein is encoded by a gene comprising a nucleic acid sequence having at least 80% identity, preferably at least 90% identity, with a sequence selected from the sequences: SEQ ID NOs: 109 to 134 (Nsp1).

In one embodiment, the invention relates to the isolated altPEP as described above, wherein the said protein is encoded by a gene comprising a nucleic acid sequence chosen from the sequences: SEQ ID NOs: 1 to 161 and 381 to 385. The invention relates in particular to the isolated altPEP as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the isolated altPEP as described above, wherein said protein is encoded by a gene comprising one selected from the sequences: SEQ ID NOs: 26 to 55 (Cpk3). In particular, the invention relates to the isolated altPEP as described above, wherein said protein is encoded by a gene comprising one selected from the sequences: SEQ ID NOs: 61 to 79 (Dcl1). In particular, the invention relates to the isolated altPEP as described above, wherein said protein is encoded by a gene comprising one selected from the sequences: SEQ ID NOs: 109 to 134 (Nsp1).

In another embodiment, the invention relates to the isolated altPEP as described above, wherein the sequence of said peptide is selected from the sequences: SEQ ID NOs: 224 to 366.

In one embodiment, the invention relates to the isolated altPEP as described above, wherein said protein is involved in at least one plant phenotype selected from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

In the invention, an altPEP can be fused or linked to one or more molecules that facilitate the entry of the altPEP into the cell. These molecules include peptides and palmitic acid. These molecules include penetrating peptides (Numata, K., et al. Library screening of cell-penetrating peptide for BY-2 cells, leaves of Arabidopsis, tobacco, tomato, poplar, and rice callus. Sci Rep 8, 10966 (2018).) and palmitic acid. Penetrating peptide” (hereinafter CPP) refers to small peptides that penetrate cellular lipid bilayers or destabilise cell membranes. CPPs can be classified into three groups: cationic, amphipathic and hydrophobic. In particular:
Cationic CPPs contain many positively charged amino acids, such as lysine (Lys) and arginine (Arg);
Amphipathic CPPs are generally composed of an alternating sequence of polar and non-polar amino acids; and
Hydrophobic CPPs are composed of non-polar amino acids with relatively low net charges.

In one embodiment, the invention relates to the isolated altPEP as described above, said isolated altPEP being fused to a peptide facilitating its entry into the plant cell. In particular, the invention relates to altPEP as described above, said altPEP being fused to a penetrating peptide.

In one embodiment, the invention relates to the isolated altPEP as described above, said isolated altPEP being fused at the N-terminus or at the C-terminus with said peptide facilitating its entry into the plant cell. In particular, the invention relates to altPEP as described above, said altPEP being fused at the N-terminus or at the C-terminus with said penetrating peptide.

In one embodiment, the invention relates to the isolated altPEP as described above, said isolated altPEP being fused with:
TAT peptide (SEQ ID NO: 380);
penetratin;
a polyhistidine peptide (in particular a peptide with at least 4 histidine residues);
or
a polyarginine peptide (in particular a peptide with 4 arginine residues).

In one embodiment, the invention relates to isolated altPEP as described above, said isolated altPEP being linked to one or more palmitic acid molecules.

In one embodiment, the invention relates to the isolated altPEP as described above, said isolated altPEP being linked at the N-terminus or at the C-terminus to one or more palmitic acid molecules.

On this point, it should be noted that the quantity of altPEP required to modulate the accumulation of a protein may vary depending on whether or not the altPEP is modified with one of the molecules facilitating its cellular penetration.

In a third aspect, the object of the above invention is a nucleic acid encoding an altPEP as described above. According to this same aspect, the invention also has as its object a nucleic acid of 3n nucleotides, which nucleic acid corresponds to a fragment of a nucleic acid sequence naturally translated onto an mRNA in a plant cell which corresponds to the open reading frame of a protein encoded by said mRNA.

In one embodiment, the invention relates to the nucleic acid as described above, said fragment comprising:
an initiation codon encoding an initiator methionine; and
a STOP codon chosen from: UAG, UGA and UAA, and said fragment being chosen in an open reading frame shifted by one or two nucleotides relative to that encoding said protein.

In one embodiment, the invention relates to the nucleic acid as described above, wherein the translation of the mRNA fragment is carried out in a reading frame different from the open reading frame of the nucleic acid sequence naturally translated in said plant cell. In particular, the invention relates to the nucleic acid as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the nucleic acid as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the nucleic acid as described above, wherein the translation of the mRNA fragment is carried out in a reading frame shifted by one (or two nucleotides at 5′) or two nucleotides at 3′ (or one nucleotide at 5′) with respect to the initiation codon of the open reading frame of the nucleic acid sequence naturally translated in said plant cell.

In particular, the invention relates to nucleic acid as described above, where n is:
from 4 to 70;
from 4 to 41;
from 5 to 40;
from 7 to 20; or
from 8 to 15.

In another aspect, the above invention relates to a composition comprising an altPEP as described above as active substance.

In another embodiment, the above invention relates to a composition comprising an altPEP as active substance, said altPEP:
having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of a protein encoded by said mRNA; and
being capable of modulating the accumulation of said protein in the plant cell and not being capable of modulating the accumulation of the mRNA encoding said protein.

In one embodiment, the invention relates to the composition as previously described, wherein said fragment comprises:
an initiation codon encoding an initiator methionine; and
a STOP codon chosen from: UAG, UGA and UAA, and said fragment being chosen in an open reading frame shifted by one or two nucleotides relative to that encoding said protein.

In one embodiment, the invention relates to the composition as described above, wherein the translation of the mRNA fragment is carried out in a reading frame different from the open reading frame of the nucleic acid sequence naturally translated in the said plant cell. In particular, the invention relates to the composition as described above, wherein the translation of the mRNA fragment is carried out in a reading frame shifted by one nucleotide at 3′ (or by two nucleotides at 5′) with respect to the open reading frame of the nucleic acid sequence naturally translated in the said plant cell. In particular, the invention also relates to the composition as described above, wherein the translation of the mRNA fragment is carried out in a reading frame shifted by two nucleotides 3′ (or one nucleotide 5′) with respect to the open reading frame of the nucleic acid sequence naturally translated in the said plant cell. In other words, the invention relates to the composition as described above, wherein the translation of the mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in the said plant cell.

In one embodiment, the invention relates to the composition as described above, wherein the said altPEP is at a concentration from 10⁻⁹ M to 10⁻³ M. On this point, it should be noted on the one hand that the composition of the invention does not exist in the natural state, and this is all the more true as such a concentration of altPEP cannot exist within a plant cell.

Furthermore, by “concentration from 10⁻⁹ M to 10⁻³ M”, is meant that the concentration of cPEP can be from 10⁻⁹ to 10⁻⁴ M, from 10⁻⁸ to 10⁻⁴ M, from 10⁻⁹ to 10⁻⁵ M, from 10⁻⁸ to 10⁻⁵ M, or from 5 μM to 500 μM, from 30 μM to 70 μM, or even 50 μM.

In particular, the invention relates to the composition as described above, wherein the said altPEP is at a concentration from 10⁻⁹ and 10⁻⁴ M, from 10⁻⁸ to 10⁻⁴ M, from 10⁻⁹ to 10⁻⁵ M or from 10⁻⁸ to 10⁻⁵ M. In particular, the invention relates to the composition as described above, wherein the said altPEP is at a concentration from 5 μM to 500 μM or from 30 μM to 70 μM. In particular, the invention relates to the composition as described above, wherein the said altPEP is at a concentration of 50 μM. Alternatively, this concentration may be 10⁻⁹ M, 10⁻⁸ M, 10⁻⁷ M, 10⁻⁶ M, 10⁻⁵ M or 10⁻⁴ M.

In view of the above, it is understood that the invention also relates to the composition comprising an altPEP as active substance, said altPEP:
having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of a protein encoded by said mRNA;
being capable of modulating the accumulation of said protein in the plant cell and not being capable of modulating the accumulation of the mRNA encoding said protein; and
being in particular at a concentration from 5 μM to 500 μM or from 30 μM to 70 μM, or being in particular at a concentration of 50 μM.

It should be noted that “composition comprising an altPEP” means that the composition of the invention comprises at least one altPEP. In other words, a mixture of altPEPs is conceivable, said altPEPs being able to target the same protein or several proteins depending on the nucleic acid fragment from which they are derived. In this respect, the aforementioned concentrations relate either to the mixture of altPEPs as such, or to each of the altPEPs in the said mixture, the said altPEPs possibly being at the same concentration or possibly being at different concentrations from those mentioned above.

In one embodiment, the invention relates to the composition as described above, said composition being a phytopharmaceutical composition, a herbicidal composition or a coating composition, in particular said coating composition further comprising at least one fixing agent.

In particular, the invention relates to the composition as described above, said composition being a phytopharmaceutical composition. In particular, the invention relates to the composition as described above, the said composition being a herbicidal composition. In particular, the invention relates to the composition as described above, said composition being a coating composition. Preferably, the invention relates to the composition as described above, the said composition being a coating composition additionally comprising at least one fixing agent.

In one embodiment, the invention relates to the composition as described above, said composition further comprising at least one solvent. Preferably, the said solvent is chosen from: acetone, acetonitrile, acetic acid, formic acid, dimethyl adipate, benzyl acetate, bi-butyl carbonate, dimethyl sulphoxide (DMSO), water, dimethyl glutarate, ammonium hydroxide, iso-butanol, iso-propanol, diethyl hexyl lactate, light aromatic naphtha solvent, heavy aromatic naphtha solvent, diethyl succinate and mixtures thereof (e.g. mixture [water; acetic acid]). g. mixture [water; acetic acid]; [acetonitrile; acetic acid], [water, acetonitrile; acetic acid], [water; DMSO], [water; acetonitrile] or [water; ammonium hydroxide]).

The solubility properties of altPEPs are determined in particular by their amino acid composition. Hydrophilic altPEPs can be solubilised and packaged in aqueous solutions, such as water. Hydrophobic altPEPs can be solubilised and packaged in solvents, such as organic solvents.

For treatment of plants with altPEPs, the organic solvents are non-toxic for the plants in small quantities, i.e. they have no deleterious effect on the development of the plant. By way of example, the organic solvents may be those mentioned above and in particular selected from acetonitrile and acetic acid.

As mentioned above, altPEPs can also be solubilised and packaged in solvent mixtures, such as, for example, an organic solvent mixture [acetonitrile; acetic acid], a mixture [water; DMSO] in a volume:volume ratio from 99:1 to 1:99, a mixture [water; acetonitrile] in a volume:volume ratio from 99:1 to 1:99 or a mixture [water; ammonium hydroxide]:volume ratio from 99:1 to 1:99, a [water; acetonitrile] mixture in a volume:volume ratio from 99:1 to 1:99 or a [water; ammonium hydroxide] mixture in a volume:volume ratio from 99:1 to 99.9:0.1. The altPEPs can also be solubilised in a solution comprising 50% acetonitrile, 10% acetic acid and 40% water (v/v/v).

In one embodiment, the invention relates to the composition as described above, said composition further comprising at least one diluent.

In one embodiment, the invention relates to the composition as described above, said composition further comprising at least one adjuvant.

In one embodiment, the invention relates to the composition as described above, said composition further comprising at least one fixing agent.

By “fixing agent” is meant a chemical or natural agent which enables the composition of the invention to adhere to a plant seed so as to coat the said plant seed. It also means a substance that makes it possible to apply and hold the active substance(s) on the seed. Available fixing agents include carboxymethyl cellulose (CMC) and gum arabic. In addition, and without limitation, a fixing agent may comprise organic solvents, water, dispersants, emulsifiers, surfactants, wetting agents and dyes.

In one embodiment, the invention relates to the composition as described above, said composition further comprising at least one plant nutrient. In particular, the invention relates to the composition as described above, the said composition additionally comprising at least one fixing agent and at least one plant nutrient.

By “plant nutrient” is meant an element assimilated by the plant to enable it to develop. By no means restrictive, a plant nutrient can be chosen from: nitrogen, phosphorus, potassium, calcium, magnesium, sulphur, manganese, iron, copper, boron, zinc, molybdenum and mixtures thereof.

In view of the foregoing, it is understood that another aspect of the invention relates to a coated seed comprising a plant seed, said plant seed being coated with a coating composition as previously described.

The coating can be produced using methodes conventionally used in the food industry and can be obtained by using a material capable of disintegrating in a solvent or in the earth, such as a binder or clay.

According to the invention, the coating can be used to confer particular properties on a seed in combination with an altPEP, such as improved growth or resistance to certain biotic or abiotic stresses.

In one embodiment, the invention relates to the coated seed as described above, wherein said plant seed to a plant species selected from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (trefoil), Medicago sativa (alfalfa), Medicago truncatula (lucerne), Nicotiana benthamiana (tobacco), Oryza sativa (rice), Pisum sativum (pea), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In one embodiment, the invention relates to coated seed as described above, said seed being treated by dipping in a composition containing an altPEP. During dipping, the seed is then totally or partially immersed in a composition containing an altPEP.

In another aspect, the invention relates to the use of an altPEP as a plant protection agent to modulate the accumulation of a protein in a plant cell, said altPEP having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of said protein encoded by said mRNA, said altPEP being capable of modulating the accumulation of said protein in the plant cell and not being capable of modulating the accumulation of the mRNA encoding said protein.

In one embodiment, the invention relates to the use of an altPEP as described above, wherein said fragment comprises:
an initiation codon encoding an initiator methionine; and
a STOP codon chosen from: UAG, UGA and UAA, and said fragment being chosen in an open reading frame shifted by one or two nucleotides relative to that encoding said protein.

In one embodiment, the invention relates to the use of an altPEP as described above, wherein the translation of the mRNA fragment is carried out in a reading frame different from the open reading frame of the nucleic acid sequence naturally translated in said plant cell. In particular, the invention relates to the use of an altPEP as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the use of an altPEP as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the use of an altPEP as described above, wherein the translation of the mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in said plant cell.

In one embodiment, the invention relates to the use of an altPEP as described above to increase the accumulation of said protein in the plant cell. The presence of the altPEP causes the amount of said protein in the treated plant cell to be greater than that in an untreated plant cell.

In one embodiment, the invention relates to the use of an altPEP as described above to decrease (inhibit) the accumulation of said protein in the plant cell. The presence of the altPEP causes the amount of said protein in the treated plant cell to be less than that in an untreated plant cell.

In one embodiment, the invention relates to the use of an altPEP as described above, wherein said altPEP is produced outside said plant cell prior to being introduced into said plant cell.

In one embodiment, the invention relates to the use of an altPEP as described above, wherein said altPEP is a synthetic peptide.

In one embodiment, the invention relates to the use of an altPEP as described above, wherein said altPEP is an isolated peptide.

In one embodiment, the invention relates to the use of an altPEP as described above, wherein said altPEP is a recombinant peptide.

In one embodiment, the invention relates to the use of an altPEP as described above, wherein said altPEP is a hydrophobic peptide or a hydrophilic peptide.

In one embodiment, the invention relates to the use of an altPEP as described above, wherein said altPEP is introduced into said plant cell in the form of a nucleic acid encoding said altPEP.

In particular, the invention relates to the use of an altPEP as described above, wherein said altPEP is introduced into said plant cell in the form of a nucleic acid encoding said altPEP and comprising the means for expressing it.

In one embodiment, the invention relates to the use of an altPEP as described above, wherein said protein is naturally present in said plant cell.

In one embodiment, the invention relates to the use of an altPEP as described above, wherein said protein is not naturally present in said plant cell. In particular, the invention relates to the use of an altPEP as described above, wherein said protein is encoded by a transgene artificially introduced into said plant cell. In particular, the invention relates to the use of an altPEP as described above, wherein said protein is encoded by a vector artificially introduced into said plant cell.

In one embodiment, the invention relates to the use of an altPEP as described above, wherein the accumulation of said protein is determined via the implementation of a technique chosen from: Western blot, measurement of enzymatic activity, mass spectrometry and translational fusion. In particular, the invention relates to the use of a alt PEP as described above, wherein the accumulation of said protein is determined via the implementation ofa Western blot.

In one embodiment, the invention relates to the use of an altPEP as described above, wherein said altPEP has a size from 4 to 41 amino acids, from 5 to 40 amino acids, from 7 to 20 amino acids or more particularly a size from 8 to 15 amino acids. In particular, said altPEP has a size from 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

In one embodiment, the invention relates to the use of an altPEP as described above, wherein said plant cell belongs to a plant species selected from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In particular, the invention relates to the use of an altPEP as described above, wherein said plant cell is an algal cell.

In one embodiment, the invention relates to the use of an altPEP as described above, wherein said protein is encoded by a gene selected from: Aae15, Aae16, abcg11, Abdcg34, Acc1, Agb1, Als, Anac076, Apg9, ArIb1, Arr1, Arr5, Arr6, At59, Bak1, Bccp1, Bccp2, Bri1, Bzo2 h3, Cesa6, Cipk3, Cks1, Cobl8, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gs/3, Hag5, Hda18, Hexo1, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Skl, Sps1, Spt, Stn8, Tap46, Topp6, TubB6, TubB8, Uba1a, Vim3, Sgr1, Abi5, Hsp101, Rh10 and Wus.

In one embodiment, the invention relates to the use of an altPEP as described above, wherein said protein is encoded by an ORF comprising a nucleic acid sequence having at least 80% identity, preferably at least 90% identity, with a sequence selected from the sequences: SEQ ID NOs: 1 to 161 and 381 to 385.

In one embodiment, the invention relates to the use of an altPEP as described above, wherein said protein is encoded by an ORF comprising a nucleic acid sequence having at least 80% identity, preferably at least 90% identity, with a sequence selected from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the use of an altPEP as described above, wherein the said protein is encoded by a gene comprising a nucleic acid sequence having at least 80% identity, preferably at least 90% identity, with a sequence chosen from the sequences: SEQ ID NOs: 26 to 55 (Cpk3). In particular, the invention relates to the use of an altPEP as described above, wherein said protein is encoded by a gene comprising a nucleic acid sequence having at least 80% identity, preferably at least 90% identity, with a sequence selected from the sequences: SEQ ID NOs: 61 to 79 (Dcl1). In particular, the invention relates to the use of an altPEP as described above, wherein said protein is encoded by a gene comprising a nucleic acid sequence having at least 80% identity, preferably at least 90% identity, with a sequence selected from the sequences: SEQ ID NOs: 109 to 134 (Nsp1).

In one embodiment, the invention relates to the use of an altPEP as described above, wherein said protein is encoded by a gene comprising a nucleic acid sequence selected from the sequences: SEQ ID NOs: 1 to 161 and 381 to 385. In one embodiment, the invention also relates to the use of an altPEP as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the use of an altPEP as described above, wherein said protein is encoded by a gene comprising one selected from the sequences: SEQ ID NOs: 26 to 55 (Cpk3). In particular, the invention relates to the use of an altPEP as described above, wherein said protein is encoded by a gene comprising one selected from the sequences: SEQ ID NOs: 61 to 79 (Dcl1). In particular, the invention relates to the use of an altPEP as described above, wherein said protein is encoded by a gene comprising one selected from the sequences: SEQ ID NOs: 109 to 134 (Nsp1).

In another embodiment, the invention relates to the use of an altPEP as described above, wherein said altPEP is selected from the sequences: SEQ ID NOs: 224 to 366.

In another embodiment, the invention relates to the use of a alt PEP as described above, wherein said protein is involved in at least one plant phenotype selected from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

In another embodiment, the invention concerns the use of an altPEP as described above, to modulate the accumulation of a recombinant protein whose nucleic acid sequence encoding it corresponds to the fusion of the nucleic acid sequences of two distinct genes.

In particular, the coding sequence of at least one of the two genes is that of a reporter gene, for example a gene encoding a fluorescent protein (such as GFP) or a protein enabling the plant to resist a compound.

In one embodiment, the invention relates to the use of an altPEP as described above, to modulate the accumulation of a recombinant protein whose nucleic acid sequence encoding it corresponds to the fusion:
a nucleic acid sequence known to be non-coding for a first gene; and
of a nucleic acid sequence coding for a second gene, the sequence of said altPEP corresponding to the translation via the genetic code of a fragment of the nucleic acid sequence deemed non-coding of the first gene.

In another aspect, the above invention relates to a method for modulating the accumulation of a protein in a plant cell comprising a step for introduction:
an altPEP; or
of a nucleic acid encoding said altPEP and the means of expressing it, in said plant cell, the introduction of said altPEP resulting in a modulation of the quantity of said protein in said plant cell, said altPEP having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of said protein encoded by said mRNA, said altPEP being capable of modulating the accumulation of said protein in the plant cell and not being capable of modulating the accumulation of the mRNA encoding said protein.

In one embodiment, the invention relates to the method as previously described, wherein said fragment comprising:
an initiation codon encoding an initiator methionine; and
a STOP codon chosen from: UAG, UGA and UAA, and said fragment being chosen in an open reading frame shifted by one or two nucleotides relative to that encoding said protein.

In one embodiment, the invention relates to the method as described above, wherein the translation of the mRNA fragment is carried out in a reading frame different from the open reading frame of the nucleic acid sequence naturally translated in said plant cell. In particular, the invention relates to the method as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the method as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the method as described above, wherein the translation of the mRNA fragment is carried out in a reading frame shifted by one (or two nucleotides at 5′) or two nucleotides at 3′ (or one nucleotide at 5′) relative to the initiation codon of the open reading frame of the nucleic acid sequence naturally translated in said plant cell.

In one embodiment, the invention relates to the method as described above, said method allowing:
promote the development of a plant; or
slow down or prevent the development of a plant.

In particular, the invention relates to the method as described above, said method making it possible to promote the development of a plant. In particular, the invention relates to the method as described above, said method making it possible to slow down or prevent the development of a plant.

In one embodiment, the invention relates to the method as described above for increasing the accumulation of said protein in the plant cell. The presence of altPEP causes the amount of said protein in the treated plant cell to be greater than that in an untreated plant cell.

In one embodiment, the invention relates to the method as described above for decreasing (inhibiting) the accumulation of said protein in the plant cell. The presence of altPEP causes the amount of said protein in the treated plant cell to be less than that in an untreated plant cell.

In one embodiment, the invention relates to the method as described above, wherein said altPEP is produced outside said plant cell before being introduced into said plant cell.

In one embodiment, the invention relates to the method as described above, wherein said altPEP is a synthetic peptide.

In one embodiment, the invention relates to the method as described above, wherein said altPEP is an isolated peptide.

In one embodiment, the invention relates to the method as described above, wherein said altPEP is a recombinant peptide.

In one embodiment, the invention relates to the method as described above, wherein said altPEP is a hydrophobic peptide or a hydrophilic peptide.

In one embodiment, the invention relates to the method as previously described, wherein said altPEP is introduced into said plant cell in the form of a nucleic acid encoding said altPEP. In particular, the invention relates to the method as described above, wherein said altPEP is introduced into said plant cell in the form of a nucleic acid encoding said altPEP and comprising the means for expressing it.

In one embodiment, the invention relates to the method as described above, wherein said protein is naturally present in said plant cell.

In one embodiment, the invention relates to the method as described above, wherein said protein is not naturally present in said plant cell. In particular, the invention relates to the method as described above, wherein said protein is encoded by a transgene artificially introduced into said plant cell. In particular, the invention relates to the method as described above, wherein said protein is encoded by a vector artificially introduced into said plant cell.

In one embodiment, the invention relates to the method as described above, wherein the accumulation of the said protein is determined using a technique selected from: Western blot, measurement of enzyme activity, mass spectrometry and translational fusion. In particular, the invention relates to the method as described above, wherein the accumulation of the said protein is determined using a Western blot.

In one embodiment, the invention relates to the method as described above, wherein said altPEP has a size from 4 to 41 amino acids, from 5 to 40 amino acids, from 7 to 20 amino acids or more particularly a size from 8 to 15 amino acids. In particular, said altPEP has a size from 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

In one embodiment, the invention relates to the method as described above, wherein said plant cell or said plant belongs to a plant species selected from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In particular, the invention relates to the method as described above, wherein said plant cell is an algal cell.

In one embodiment, the invention relates to the method as described above, wherein said protein is encoded by a gene selected from: Aae15, Aae16, abcg11, Abdcg34, Acc1, Agb1, Als, Anac076, Apg9, Arlb1, Arr1, Arr5, Arr6, At59, Bak1, Bccp1, Bccp2, Bri1, Bzo2 h3, Cesa6, Cipk3, Cks1, Cob18, Coi1, Cpk3, Crk34, Cyp705a18, Cyp71b26, Cyp78a8, Cyp97b3, Dcl1, Dur3, Ein2, Emb175, Emb2726, Emb9, Epsps, Fnr1, Fve, Ga2ox7, Gapc, Gcn2, Gdi2, Gln2, Gsl3, Hag5, Hda18, Hexo1, Hppd, Hsl1, Iaa31, Iqd28, Jac1, Jar1, Kp1, Lrx2, Mapkkk3, Mapkkk5, Mfp2, Mrb1, Nsp1, Pds, Pen3, Phyb, Pif3, Pizza, Ppox1, Ppox2, Prp39, PsbA, Pskr1, Rd21, Ring1, Ros1, Rpt4a, Sfr6, Shr, Shy2, Skl, Sps1, Spt, Stn8, Tap46, Topp6, TubB6, TubB8, Uba1a, Vim3, Sgr1, Abi5, Hsp101, Rh10 and Wus.

In one embodiment, the invention relates to the method as described above, wherein said protein is encoded by an ORF comprising a nucleic acid sequence having at least 80% identity, preferably at least 90% identity, with a sequence chosen from the sequences: SEQ ID NOs: 1 to 161 and 381 to 385.

In one embodiment, the invention relates to the method as described above, wherein said protein is encoded by an ORF comprising a nucleic acid sequence having at least 80% identity, preferably at least 90% identity, with a sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method as described above, wherein the said protein is encoded by a gene comprising a nucleic acid sequence having at least 80% identity, preferably at least 90% identity, with a sequence chosen from the sequences: SEQ ID NOs: 26 to 55 (Cpk3). In particular, the invention relates to the method as described above, wherein said protein is encoded by a gene comprising a nucleic acid sequence having at least 80% identity, preferably at least 90% identity, with a sequence chosen from the sequences: SEQ ID NOs: 61 to 79 (Dcl1). In particular, the invention relates to the method as described above, wherein said protein is encoded by a gene comprising a nucleic acid sequence having at least 80% identity, preferably at least 90% identity, with a sequence chosen from the sequences: SEQ ID NOs: 109 to 134 (Nsp1).

In one embodiment, the invention relates to the method as described above, wherein said protein is encoded by a gene comprising a nucleic acid sequence chosen from the sequences: SEQ ID NOs: 1 to 161 and 381 to 385. In one embodiment, the invention also relates to the method as described above, wherein the said protein is encoded by a gene comprising a nucleic acid sequence chosen from the sequences: SEQ ID NOs: 26 to 55 (Cpk3), SEQ ID NOs: 61 to 79 (Dcl1) and SEQ ID NOs: 109 to 134 (Nsp1).

In particular, the invention relates to the method as described above, wherein said protein is encoded by a gene comprising one selected from the sequences: SEQ ID NOs: 26 to 55 (Cpk3). In particular, the invention relates to the method as described above, wherein said protein is encoded by a gene comprising one selected from the sequences: SEQ ID NOs: 61 to 79 (Dcl1). In particular, the invention relates to the method as described above, wherein the said protein is encoded by a gene comprising one selected from the sequences: SEQ ID NOs: 109 to 134 (Nsp1).

In another embodiment, the invention relates to the method as described above, wherein said altPEP is selected from the sequences: SEQ ID NOs: 224 to 366.

In another embodiment, the invention relates to the method as described above, wherein said protein is involved in at least one plant phenotype selected from:
size, shape, surface area, volume, mass and number of leaves;
size, shape, surface area, volume, mass and number of flowers;
pruning the stem (or flower stalk);
root biomass;
the number, length and degree of branching of the roots;
early germination;
earliness of budding;
the earliness of floral induction (or floral transition);
germinative vigour and the duration of the juvenile phase;
duration of flowering;
resistance to biotic stress;
resistance to abiotic stress; and
the number of cells.

In one embodiment, the invention relates to the method as described above, wherein the introduction of said altPEP leads to earlier bolting in said plant.

In one embodiment, the invention relates to the method as described above, wherein the introduction of said altPEP results in earlier flowering in said plant.

In one embodiment, the invention relates to the method as described above, wherein introduction of said altPEP results in an increase in stem size in said plant.

In one embodiment, the invention relates to the method as described above, wherein the introduction of said altPEP results in earlier stem growth in said plant.

The Inventors have unexpectedly found that it is possible to apply an altPEP directly to the plant, e.g. via the use of the composition of the invention (see above) comprising an altPEP, to modulate the accumulation of a target protein in the plant, which indicates that the altPEP is taken up by the plant.

Therefore, in one embodiment, the invention relates to the method as described above, wherein said altPEP is introduced into said plant:
by watering, by spraying or by adding a fertiliser, a potting soil, a culture substrate or a support in contact with the plant, the said altPEP being administered to the plant in particular in the form of a composition comprising from 10⁻⁹ M to 10⁻⁴ M of the said altPEP;
by watering, soaking, spraying or by adding a fertiliser, a potting soil, a growing substrate or a support in contact with the plant, the said altPEP being administered in particular to a seed or a seedling in the form of a composition comprising from 10⁻⁹ M to 10⁻⁴ M of the said altPEP; or
by means of a nucleic acid encoding said altPEP and comprising the means for expressing said altPEP, said nucleic acid being artificially introduced into the plant.

In one embodiment, the invention relates to the method as defined above, wherein said altPEP is artificially introduced externally into the plant, preferably by watering, spraying or by the addition of a fertiliser, potting soil, growing substrate or inert support.

In one embodiment, the invention relates to the method as defined above, wherein said altPEP is introduced by watering.

In one embodiment, the invention relates to the method as defined above, wherein said altPEP is introduced by spraying.

In one embodiment, the invention relates to the method as defined above, wherein said altPEP is introduced by the addition of a fertiliser.

In one embodiment, the invention relates to the method as defined above, wherein the plant is treated with a composition comprising from 10⁻⁹ M to 10⁻⁴ M of said altPEP, or comprising in particular 10⁻⁹ M, 10⁻⁸ M, 10⁻⁷ M, 10⁻⁶ M, 10⁻⁵ M or 10⁻⁴ M of said altPEP. Preferably, the compositions have a concentration of 10⁻⁸ M to 10⁻⁵ M for application to the plant by watering or spraying.

In addition, more or less concentrated compositions can be used to treat the plant with altPEP. For example, and without limitation, more concentrated compositions comprising from 10⁻¹ M to 10⁻³ M, or comprising in particular 10⁻² M of altPEP, can be used in the case where the altPEP artificially introduced externally is administered to the plant by spreading.

In another aspect, the above invention concerns a modified plant containing an altPEP, which “modified plant” corresponds to a plant into which an altPEP has been artificially introduced, in particular by watering, spraying or via a fertiliser.

In one embodiment, the invention relates to the modified plant comprising an exogenously introduced altPEP, said altPEP having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of a protein encoded by said mRNA, said altPEP being capable of modulating the accumulation of said protein in the plant cell and not being capable of modulating the accumulation of the mRNA encoding said protein.

In one embodiment, the invention relates to the modified plant as described above, wherein said fragment comprises:
an initiation codon encoding an initiator methionine; and
a STOP codon chosen from: UAG, UGA and UAA,
   and said fragment being chosen in an open reading frame shifted by one or two nucleotides relative to that encoding said protein.

In one embodiment, the invention relates to the modified plant as described above, wherein the translation of the mRNA fragment is carried out in a reading frame different from the open reading frame of the nucleic acid sequence naturally translated in said plant cell. In particular, the invention relates to the modified plant as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the modified plant as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the modified plant as described above, wherein the translation of the mRNA fragment is carried out in a reading frame shifted by one (or two nucleotides at 5′) or two nucleotides at 3′ (or one nucleotide at 5′) with respect to the initiation codon of the open reading frame of the nucleic acid sequence naturally translated in said plant cell.

In one embodiment, the invention relates to the modified plant as described above, said plant belonging to a plant species selected from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In another aspect, the above invention relates to a transgenic plant comprising a nucleic acid encoding an altPEP and the means of expressing it,
said altPEP having a size from 4 to 70 amino acids, in particular from 4 to 41 amino acids, the amino acid sequence of which corresponds to the translation via the genetic code of a fragment of a nucleic acid sequence naturally translated on an mRNA in a plant cell which corresponds to the open reading frame of a protein encoded by said mRNA,
said altPEP being capable of modulating the accumulation of said protein in the plant cell and not being capable of modulating the accumulation of the mRNA encoding said protein.

In one embodiment, the invention relates to the transgenic plant as described above, wherein said fragment comprises:
an initiation codon encoding an initiator methionine; and
a STOP codon chosen from: UAG, UGA and UAA,
   and said fragment being chosen in an open reading frame shifted by one or two nucleotides relative to that encoding said protein.

In one embodiment, the invention relates to the transgenic plant as described above, wherein the translation of the mRNA fragment is carried out in a reading frame different from the open reading frame of the nucleic acid sequence naturally translated in said plant cell. In particular, the invention relates to the transgenic plant as described above, wherein the translation of the mRNA fragment is carried out in a reading frame shifted by one nucleotide at 3′ (or by two nucleotides at 5′) with respect to the open reading frame of the nucleic acid sequence naturally translated in said plant cell. In particular, the invention also relates to the transgenic plant as described above, wherein the translation of the mRNA fragment is carried out in a reading frame shifted by two nucleotides 3′ (or one nucleotide 5′) with respect to the open reading frame of the nucleic acid sequence naturally translated in said plant cell. In other words, the invention relates to the transgenic plant as described above, wherein the translation of the mRNA fragment is carried out in a reading frame shifted by one (or two nucleotides at 5′) or two nucleotides at 3′ (or one nucleotide at 5′) with respect to the initiation codon of the open reading frame of the nucleic acid sequence naturally translated in the said plant cell.

In one embodiment, the invention relates to the transgenic plant as defined above, wherein the sequence encoding said altPEP is shorter than the sequence of the mRNA encoding said protein.

In one embodiment, the invention relates to the transgenic plant as described above, said plant belonging to a plant species selected from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (trefoil), Medicago sativa (alfalfa), Medicago truncatula (lucerne), Nicotiana benthamiana (tobacco), Oryza sativa (rice), Pisum sativum (pea), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

In one embodiment, the invention relates to the transgenic plant as described above, wherein expression of said altPEP is placed under the control of a strong promoter, preferably a constitutive strong promoter such as the 35S promoter.

In any event, it should be noted that the various aspects of Invention No. 2, like the various embodiments thereof, are interdependent. They may therefore be combined to obtain aspects and/or preferred embodiments of Invention No. 2 not explicitly described. This also applies to the set of definitions provided in this description, which applies to all aspects of Invention No. 2 and its embodiments.

In addition, inventions No. 1 and No. 2 are illustrated by, but not limited to, the following Figures and Examples.

### LIST OF FIGURES

FIG. 1

FIG. 1 is a schematic representation illustrating the first steps of the method for preparing and determining a cPEP. In particular, the steps for determining within a protein mRNA one of the nucleic acid sequences from which the peptide to be tested (i.e. the potential cPEP) is identified are illustrated.

The sequences: SEQ ID NOs: 375 to 379 are provided as examples only.

FIG. 2

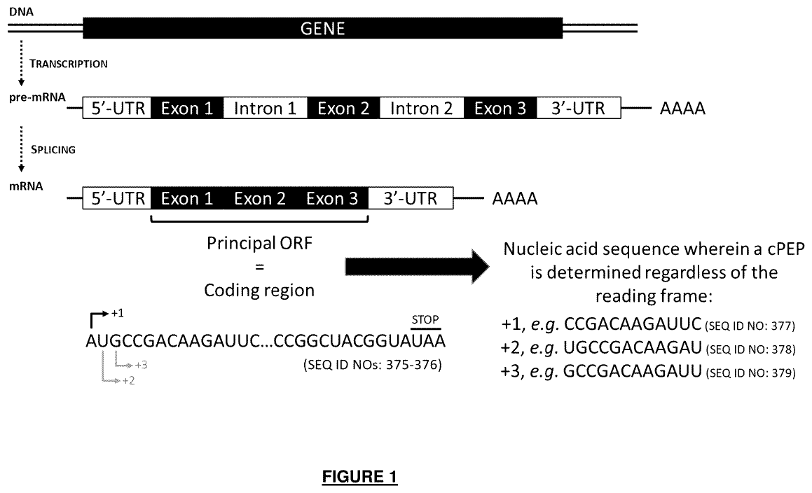

Identification and characterisation of altPEPs

(a) Schematic representation of a gene encoding altORFs producing altPEPs. (b) ORF ranking distribution of altPEPs detected by MS. (c) Frequency of altPEPs detected by MS by length ranges. (d) Main root length of A. thaliana seedlings treated with 100 μM peptide for 4 days. (e, f) Quantification of DCL1 expression by RT-qPCR (e) and Western blot using anti-DCL1 antibodies (f) in A. thaliana leaves treated for 5 days with 100 μM peptide. (g, h) Quantification of COI1, AP2, CPK3 (g) and EIN2 (h) by Western blot using specific antibodies in A. thaliana leaves treated for 5 days with 100 μM peptide.

Results are representative of four independent experiments. Figures indicate mean and SEM. Controls are irrelevant peptide and water. Error bars represent SEM, asterisks indicate a significant difference between test condition and control according to Student's t-test (d) or Wilcoxon's t-test (e-h) (n=100 (d), n=4 (e-h); p<0.05).

### FIG. 3

(a) Distribution of altPEPs detected by MS as a function of the number of Brassicaceae species wherein homologues (more than 80) were found. (b) Distribution of altPEPs detected by MS as a function of their encoding framework. (c) Alignment of AtDCL1alt18 (SEQ ID NO: 224) homologues in different Brassicaceae species. (d, e) Conservation of the first altPEP of AP2 (d) and EIN2 (e) in different Brassicaceae species. (f) EIN2 refAUG activity compared to altAUG1 activity performed with the in vitro transcription/translation system.

At: Arabidopsis thaliana, Al: Arabidopsis lyrata, Bn: Brassica napus, Bo: Brassica oleracea, Br: Brassica rapa, Bs: Boechera stricta, Cg: Capsella grandiflora, Cr: Capsella rubella, Cs: Camelina sativa, Es: Eutrema salsugineum, Rs: Raphanus sativus, Th: Thellungiella halophila.

FIG. 4

Quantification by qRT PCR of the expression of different genes in response to 100 μM of different peptides

(a) Treatment of plants with the peptide AtCOI1alt1; (b) Treatment of plants with the peptide AtAP2alt1; (c) Treatment of plants with the peptide AtEIN2alt1 (d) Treatment of plants with the peptide AtCPK3alt1.

Error bars represent SEM.

FIG. 5

Complementary peptides increase protein expression

Analysis of luciferase expression in A. thaliana constitutively expressing the LUC transgene. (a) Agarose gel after PCR amplification of RNA obtained by immunoprecipitation using anti-HA magnetic beads, followed by RT-PCR, on plants treated with the indicated peptide (SC-HA: Scrambled cPEPluc-HA; Luc-HA: cPEPluc-HA). (b) Relative expression of the luciferase transgene in plants treated with the indicated peptide, quantified by RT-qPCR. (c-e) Relative quantification of LUC in plants treated with the indicated peptide. (f, g) Ratio between induction and quantification of LUC in plants treated with cPEPluc compared to plants treated with Scrambled cPEPluc, using different peptide concentrations (f) or taken at different time points (g). (h) Transcriptomic analysis of plants treated with cPEPluc-HA compared to plants treated with Scrambled cPEPluc-HA. (i) Proteomic analysis of plants treated with cPEPluc-HA compared to plants treated with Scrambled cPEPluc-HA. Protein expression was analysed by Western blots and quantified using ImageJ.

Error bars represent SEM, asterisks indicate a significant difference between test condition and control according to the Wilcoxon test (a, h, i, n=5; c, n=6; b, d-g, n=12; p<0.05).

FIG. 6

cPEPs require sequence complementarity with their target for their activity to be effective

(a) FRET-FLIM analysis of the interaction between cPEPnsp1-FAM and NSP1 (left) or NSP1ΔcPEP (right) in planta. (b) qRT-PCR quantification of NSP1 expression in M. truncatula roots treated with cPEPnsp1 or Scrambled cPEPnsp1. (c) GUS quantification of peptide-treated M. truncatula roots carrying the Pro NSP1-NSP1-GUS fusion. (d, e) Quantification of NSP1 expression in N. benthamiana leaves after infiltration of different constructs: ‘wild type’ i.e. version of NSP1 wherein the cPEP sequence has been deleted (NSP1 ΔcPEP) or version of NSP1 wherein the cPEP sequence has been replaced by an artificial sequence (NSP1 ΔcPEP-cPEPartificial), with an empty vector (control) or cPEPnsp1 or cPEPartificial. (f, g) Formation of lateral roots of M. truncatula WT seedlings, nsp1 or overexpressing NSP1, in response to cPEPnsp1 or Scrambled cPEPnsp1. (h, i) Quantification of nodules on roots of M. truncatula roots treated with an irrelevant peptide or cPEPskl. (j) Root development of M. truncatula seedlings infected with A. euteiches and treated with an irrelevant peptide or cPEPrh10. (k) Relative expression of A. euteiches α-tubulin in M. truncatula seedlings infected with A. euteiches and treated with an irrelevant peptide or cPEPrh10.

Error bars represent SEM, asterisks indicate a significant difference between test condition and control according to Student's t-test (f-i, j) or Wilcoxon test (b-e, k) (b-e, k, n=6; f-i, n=50; j, n=60; p<0.05).

FIG. 7

cPEPs can modulate the development of A. thaliana and its response to stresses

(a, b) Length of primary roots of A. thaliana seedlings treated with a Scrambled cPEPdcl1 or with a cPEPdcl1. (c) Relative chlorophyll content of A. thaliana seedlings treated with the corresponding peptide. (d) Resumption of growth of A. thaliana seedlings after heat shock at 45° C. for 45 min and treated with the corresponding peptide. (e) Relative lesion area of A. thaliana seedlings infected with B. cinerea and treated with the indicated peptide. (f) Measurement of the flowering day of A. thaliana plants treated with the indicated peptides. (g, h) Flowering day of A. thaliana plants treated with an irrelevant peptide or a mixture of cPEPs (targeting EIN2, BR11, BAK1 and WUS). (i, j) Leaf area of A. thaliana plants treated with an irrelevant peptide or a mixture of cPEPs (targeting EIN2, BR11, BAK1 and WUS).

Error bars represent SEM, asterisks indicate a significant difference between test condition and control according to Student's t-test (b, c, e, f, h, j) (b, n=50; c, e, f, h, j, n=40; d, n=5; p<0.05).

FIG. 8

cPEPs can modulate the development of A. thaliana and its response to stresses

(a) Quantification of CPK3 expression, using CPK3 antibodies, in peptide-treated A. thaliana plants. (b-c) Infection assay of peptide-treated A. thaliana leaves inoculated with B. cinerea spores.

Error bars represent SEM, asterisks indicate a significant difference between the test condition and the control according to the Wilcoxon test (a) and Student's t-test (c) (a: n=6; c, n=50; p<0.05).

FIG. 9

cPEP increases protein translation

(a) Analysis of luciferase activity in A. thaliana plants constitutively expressing the LUC transgene and treated with Scrambled cPEPluc or cPEPluc, with or without cycloheximide. (b) Analysis of luciferase activity after in vitro transcription/translation of luciferase expressed with cPEPluc or Scrambled cPEPluc.

Error bars represent SEM, asterisks indicate a significant difference between test condition and control according to the Wilcoxon (a-b) test (a, b, n=6; p<0.05).

FIG. 10

cPEPs are useful tools in agronomy

(a, b) Relative area of lesions on S. lycopersicum leaves infected with B. cinerea and treated with an irrelevant peptide or cPEPjar1. (c, d) Heat stress resistance of soybean plants treated with an irrelevant peptide or cPEPsp101. (e, f) Growth (plant height) of soybean plants treated with an irrelevant peptide or a mixture of cPEPs (targeting MRB1, SHY2 and SGR1). (g, h) Leaf area of B. vulgaris plants treated with an irrelevant peptide or a mixture of cPEPs (targeting EIN2, BR11, BAK1 and WUS). (i, j) Leaf surface of A. hypochondriacus plants treated with an irrelevant peptide or a mixture of cPEPs (targeting EIN2, BR11, BAK1 and WUS).

Error bars represent SEM, asterisks indicate a significant difference between test condition and control according to Student's t-test (b, d, f, h, j, n=40; p<0.05).

### EXAMPLES

### Materials & Methods

### Biological Material and Growth Conditions

Medicago truncatula Gaertn cv. Jemalong genotype A17 were grown on Long Aston medium as described in Delaux P M et al (New Phytol. 2013 July; 199(1):59-65). Arabidopsis thaliana Col-0 plants were grown in soil until 4 weeks of age in a growth chamber (22/20° C., 16 h/8 h L/D, RH 80%, ~75 μmol·m⁻² s⁻¹). Barbarea vulgaris seeds were stratified for 24 h at 4° C. before being grown in pots in a growth chamber. Seeds of Amaranthus hypochondriacus, Glycine max and Solanum lycopersicum were sown in pots and grown in a growth chamber. Nicotiana benthamiana and M. truncatula plants were grown as described in Combier J P et al (Genes and Development. 22, 1549-1559, 2008). ABRE-LUC seedlings were provided by MR Knight (Arabidopsis. Plant Cell. 23, 4079-95, 2011). Arabidopsis thaliana LUC seedlings were sterilised and sown in 96-well plates containing 100 μL of MS/2 medium.

### Peptides

Peptides with sequences SEQ ID NOs: 162 to 223 and 386 to 418 were synthesised by Smart Biosciences and dissolved at a concentration of 2 to 10 mM in water, aliquoted and stored at −80° C.

### Plasmid Constructs

Plasmids were obtained using the Golden Gate cloning strategy in modified pCAMBIA220. Mt-NSP1 (Medtr8g020840):GUS translational fusion was performed using 3183 bp of the NSP1 promoter and 3180 bp of the post CDS section of NSP1. Expression in N. benthamiana leaves was performed using the 35S promoter.

### Plant Methoding

Nicotiana benthamiana leaves were transformed according to Combier J P et al (Genes and Development. 22, 1549-1559, 2008).

### Gene Expression Analysis

Quantification of mRNAs was performed by qRT-PCR using appropriate primer pairs selected from the sequences: SEQ ID NOs: 367 to 374. Expression levels for the controls were set at 100. The primers used for the A. euteiches assays are described in Camborde et al (New Phytol. 233:2232-2248. 2022).

### Pathogenicity Tests

Wild-type or mutant A. thaliana leaves were sprayed daily with 100 μM of peptide or 100 μM of the corresponding scramble version for 3 days. Six hours after the last treatment, five mature leaves per plant were inoculated with a 5 μL droplet of 2.5×10⁵ spores/mL of Botrytis cinerea strain B05.10 diluted in 100 μM of peptide or its corresponding scramble version. After inoculation, plants were maintained at 100% relative humidity. Then, a 2 μL droplet of 100 μM of peptide or 100 μM of the corresponding scramble version was deposited daily on B. cinerea-infected leaves for 3 days (until symptoms appeared). Leaves were removed from the plants to determine lesion areas using the ImageJ programme. For tomato, the protocol was the same except that inoculation was carried out with 5,000 B. cinerea spores, without peptides. Peptides were added 1 h after inoculation (1 μL of 500 μM) and each day for two days with 5 μL of 100 μM peptide. For infection of M. truncatula with A. euteiches, plants were grown on agar medium and treated with 10 μL of 100 μM peptide, 24 h before, 24 h and 72 h after infection with 10 μL (1000 spores) of A. euteiches spores. Plants were harvested 7 days after inoculation for RNA extraction.

Treatment with cPEP

N. benthamiana plants were treated by spraying the leaves 24 h before harvesting. For Luc assays, 100 μL of MS/2 liquid medium containing the peptides was added to each well. 5 μL of luciferin was added and luciferase activity was read 30 min later using a spectrophotometer. All other assays were performed by spraying or watering the plants. To quantify the protein level induced by cPEPs in Arabidopsis, rosettes were sprayed daily with 100 μM of peptide or its control for 5 days. Leaves were harvested 6 h after the last treatment for western blot analysis. For the flowering assay and chlorophyll content, 10-day-old Arabidopsis plants were sprayed with 500 μL of 10 μM peptide three times a week until flowering. Chlorophyll content was measured using the SPAD chlorophylometer (Konica Minolta). Leaf area was measured using ImageJ software. Barbarea vulgaris and A. hypochondriacus seedlings were treated immediately after sowing and 3 times a week with 500 μL of a 20 μM mixture of each peptide. For western blotting, a heat shock was performed by placing 20-day-old plants grown in 24-well plates on MS/2 for 90 min at 37° C. before harvesting. The plants were treated with 100 μM of peptide for 24 h. For the heat shock resistance test on A. thaliana, 3-day-old seedlings were treated for three days with 100 μM of peptide before placing the plants at 45° C. for 45 min. The plants were placed in a growth chamber to recover and treated 24 h after heat shock with 100 μM of peptide. For soybean heat shock, one-week-old seedlings were treated for 48 h, 24 h and 30 min before being placed at 45° C. for 24 h. For soybean growth, seedlings were treated 3 times a week for a fortnight with 500 μL of 100 μM peptide.

### Protein Extraction and Western Blot Analysis

Total proteins from Arabidopsis plants were extracted according to Ormancey et al (Plant Sci. 2019 March; 280:12-17). Western blot analyses were performed according to Combier et al. (Genes Dev. 2008 Jun. 1; 22(11):1549-59). Protein levels were normalised to Ponceau staining. Antibodies were purchased from Agrisera and Sigma.

### In Vitro Transcription/Translation

The TnT® SP6 high-throughput wheat germ protein expression system (Promega) was used according to the manufacturer's instructions. The LUC sequence was amplified using the GoTaq Polymerase® (Promega), cPEPs or their corresponding Scramble versions were added just prior to the start of the reaction. The reaction was stopped after 60 min, after which LUC activity was measured using a plate spectrophotometer (Perkin-Elmer Victor Nivo).

Treatment with Cycloheximide

Arabidopsis thaliana plants comprising the LUC construct were treated by infiltrating the leaves with a solution of peptide with or without cycloheximide (200 μg·mL⁻¹) in MS/2 medium, 24 h before harvesting for the LUC assay.

### RNA Immunoprecipitation

RNA co-immunoprecipitation was adapted from Merret et al. (Plant Physiol. 174:1216-1225. 2017). 400 mg of tissue powder was incubated in 3 mL of lysis buffer (200 mM Tris, pH 9.0, 110 mM potassium acetate, 0.5% Triton X-100, 0.1% Tween© 20, 5 mM DTT, 1.5% protease inhibitor and 80 units·ml-1 RNasin). The lysate was incubated on ice for 10 minutes and then centrifuged at 16,000 g for 10 minutes at 4° C. 1.5 mL of crude extract was incubated with 25 μL of anti-HA magnetic beads (Thermo Scientific) for 1.5 h at 4° C. under rotation. After binding, the beads were washed five times with 0.75 mL of lysis buffer. Elution was performed with 200 μL of 8 M guanidium for 5 min on ice and precipitated overnight with 300 μL of 100% ethanol. After centrifugation (16,000 g, 45 min, 4° C.), pellets were resuspended in 200 μL of Monarch DNA/RNA Protection Reagent (New England Biolabs) and RNA was extracted according to the manufacturer's instructions and concentrated into 10 μL using the Monarch RNA Cleanup Kit© (New England Biolabs). 300 μL of input and unbound fractions were retained and RNA was extracted as described above. Reverse transcription was performed on 10 μL of eluate or 500 ng input/unbound using the Superscript© IV kit (Thermo Scientific). PCR amplification was performed on 1 μL of cDNA with specific primers.

### FRET FLIM Analysis, Preparation of Leaf Samples for Testing

Samples were prepared based on the protocols of Camborde et al (Nat Protoc. 12:1933-1950. 2017). Briefly, Agrobacterium tumefaciens strain GV3101pmp90 carrying 35S-NSP1 or 35S-NSP1ΔORF plasmids were used to infiltrate N. benthamiana leaves. Agro-infiltrated discs from at least three different leaves were fixed after 48 h by vacuum infiltration of a 4% (w/v) paraformaldehyde fixation solution, followed by a permeabilization step using proteinase K treatment. After washing, nucleic acid staining was performed by vacuum infiltration of a 5 μM Sytox Orange solution (Invitrogen). The discs were then washed and mounted on TBS prior to FLIM measurements of the nuclei.

FRET FLIM Analysis, Preparation of cPEP Sheet Samples for FRET-FLIM Experiments

Plasmids 35s-NSP1 or 35s-NSP1ΔcPEP were transformed into A. tumefaciens strain pmp90 GV3101 and agroinfiltrated into N. benthamiana leaves. 40 h later, a 10 μM solution of cPEP-FAM was infiltrated into the same leaves and plants were incubated for 3 h. Next, leaf discs were fixed and treated as described in Camborde et al (Nat Protoc. 12:1933-1950. 2017), then washed and mounted on TBS prior to cytoplasmic FLIM measurements.

### FRET FLIM Analysis, TCSPC-FLIM Data Acquisition

FLIM was performed on a Leica TCS SP8 SMD, which consists of a LEICA DMi8 inverted microscope equipped with a PicoQuant TCSPC system. Excitation of the FITC donor at 470 nm was performed by a picosecond pulsed diode laser at a repetition rate from 40 MHz, through an oil immersion objective (63×, N.A. 1.4). The emitted light was detected by a Leica HyD detector in the emission range 500-550 nm. Images were acquired with acquisition photons of up to 1500 per pixel.

### FRET FLIM Analysis, FLIM Data Analysis

From the fluorescence intensity images, decay curves were calculated per pixel and fitted (by Poisson maximum likelihood estimation) with a mono- or double-exponential decay model using SymphoTime 64 software (PicoQuant, Germany). The single-exponential model function was applied for donor samples with only FITC present. The double-exponential model function was used for samples containing FITC and Sytox. The experiments were repeated at least three times to obtain statistically valid data. Energy transfer efficiency (E) based on fluorescence lifetime (T) was calculated as follows: E=1−(τD+A/τD−A), where τD+A is the fluorescence lifetime of the donor in the presence of the acceptor and τD-A is the fluorescence lifetime of the donor in the absence of the acceptor.

### Statistical Analysis

Mean values of relative gene expression, protein level or phenotypic parameters were compared using the Wilcoxon or Student's t-test. Error bars represent the standard error of the mean (SEM). Asterisks indicate significant differences (p<0.05).

### Results

AltPEPs Control the Translation of their Coding Gene

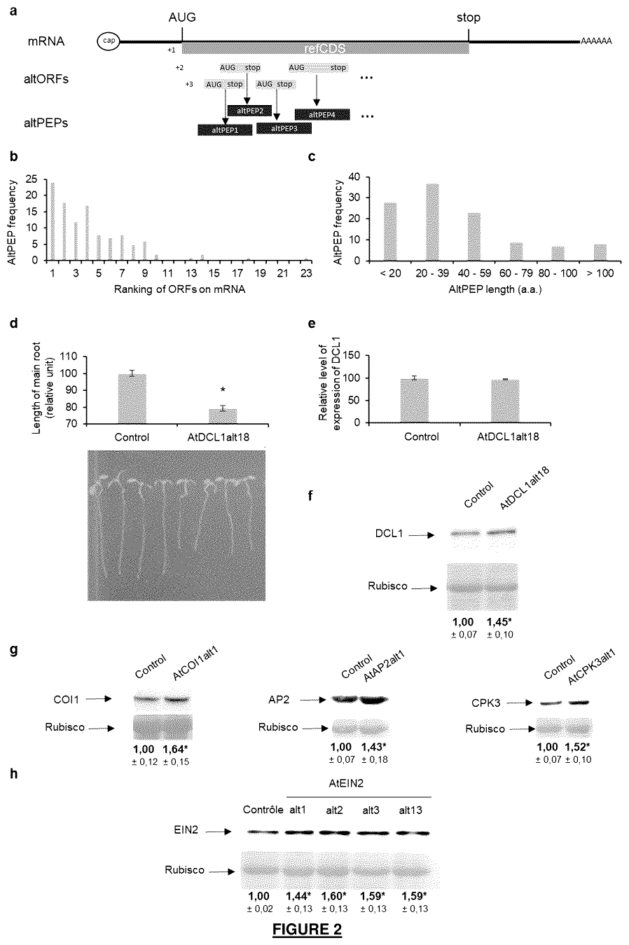

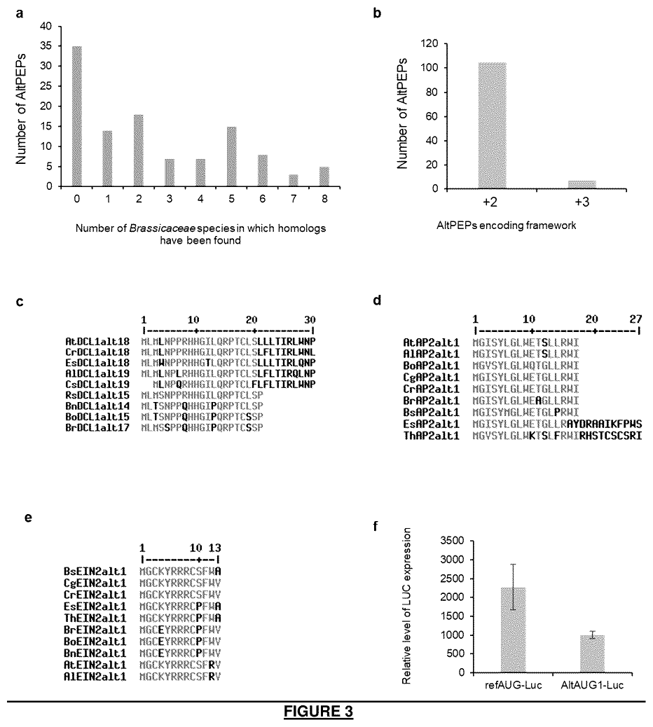

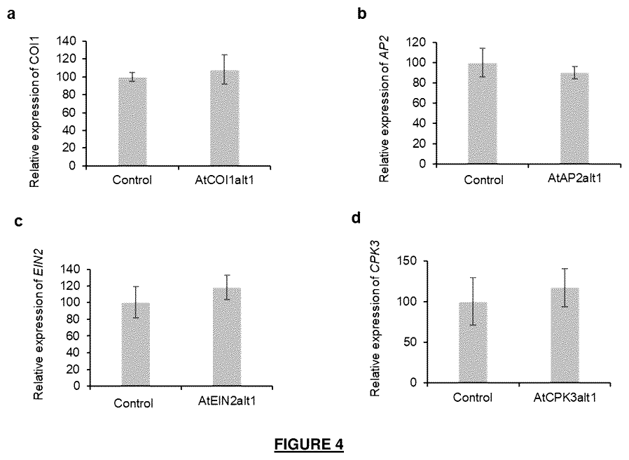

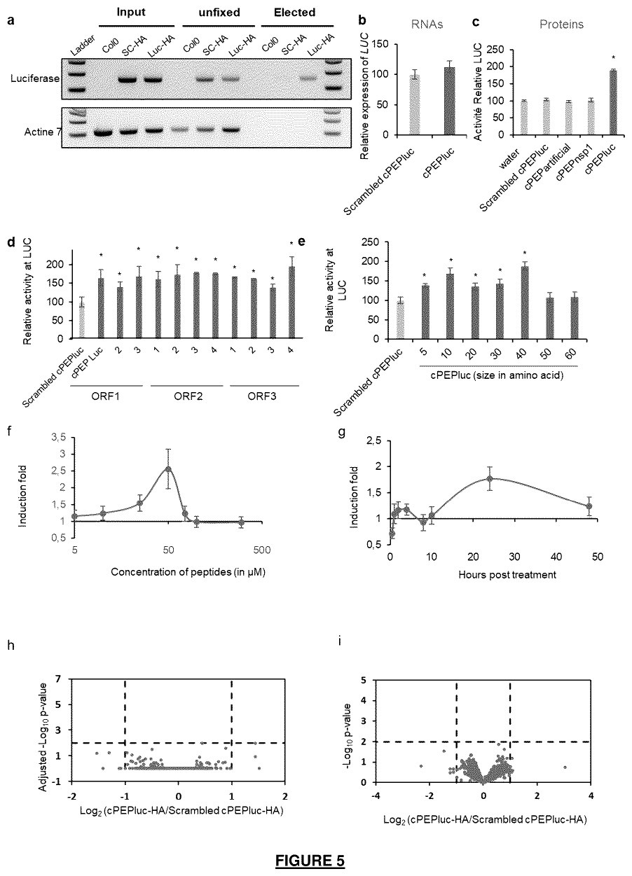

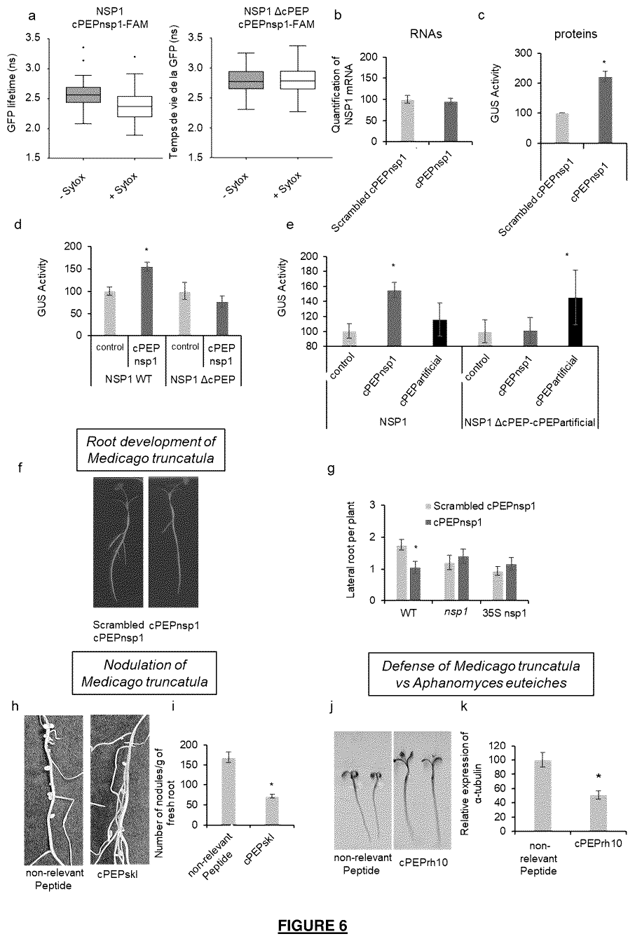

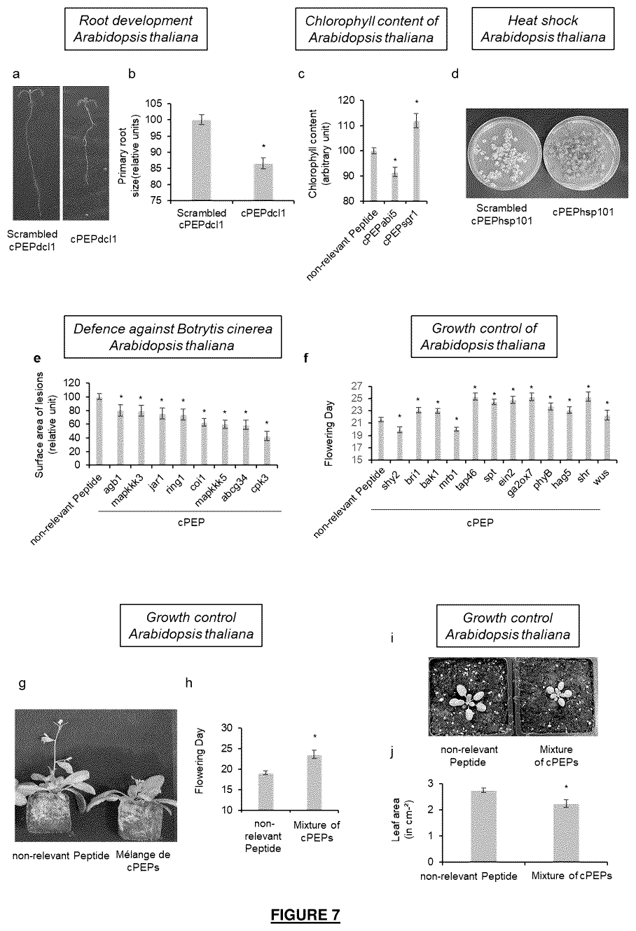

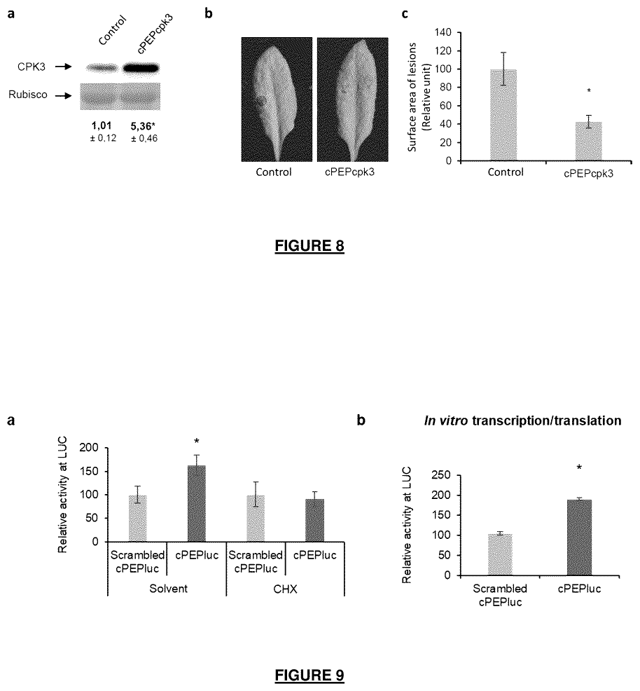

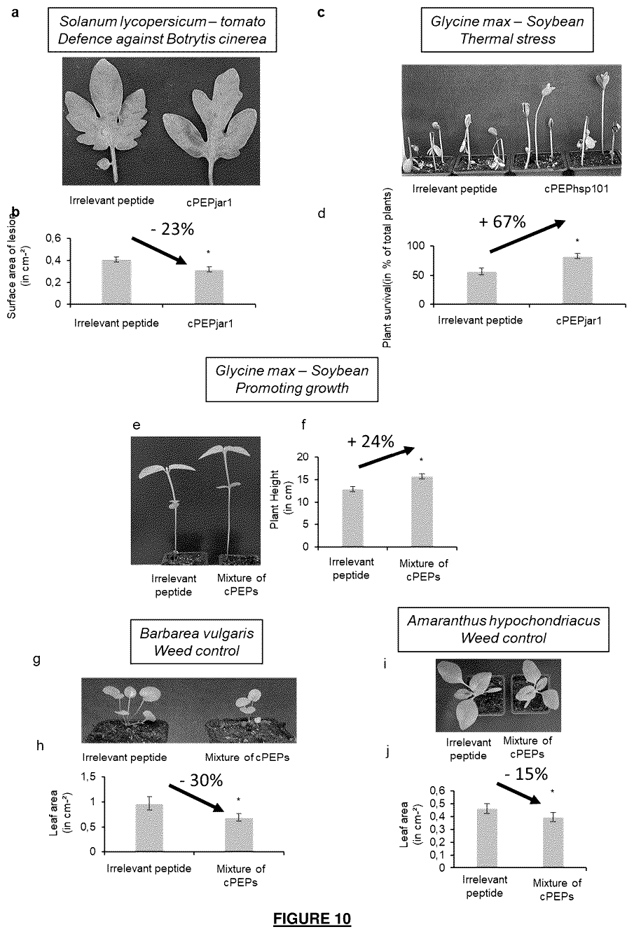

The first objective was to examine whether natural altPEPs could be detected in A. thaliana (FIG. 2a). To do this, a list of all potential altORFs longer than 10 amino acids and located out-of-frame in the coding sequences of A. thaliana was generated bioinformatically. Based on this list, we analysed a recently published MS dataset (Müller J. B. et al. Nature. 582, 592-596 (2020)) and identified 112 new altPEPs, showing that altPEPs are naturally represented in the in planta proteome (SEQ ID NOs: 252-363). At the same time, 85 of the 112 corresponding reference proteins were identified in the same dataset, suggesting that altPEPs could in some cases be more highly expressed than their reference proteins. Of the 112 altPEPs identified, 24 corresponded to the first altORF found in the coding sequence (FIG. 2b; SEQ ID NOs: 258, 261, 262, 263, 270, 274, 284, 285, 286, 290, 295, 303, 312, 316, 320, 321, 323, 324, 332, 343, 344, 352, 353 and 358). Surprisingly, the majority of altPEPs identified were encoded from other alternative AUGs (altAUGs), from the second to the 23rd^{ème}, showing that several altAUGs, located downstream of the canonical AUGs, can be translated and be active in planta (FIG. 2b). The average length of the peptides encoded by the altORFs detected was 45 amino acids, while the longest contained 212 amino acids (FIG. 2c, SEQ ID NO: 295). Interestingly, 35 altPEPs shared no homology within the Brassicaceae (FIG. 3a). Finally, it is surprising that 105 out of 112 altPEPs are encoded by altAUGs located in frame 2 (considering that the main protein is encoded by frame 1) (FIG. 3b). These altPEPs have not been described by others (Wang S. et al. Mol Plant 13, 1-16 (2020)), providing additional translated peptides to those already described. This analysis showed that altPEPs are naturally expressed at detectable levels in planta, and that they are probably widespread among the various annotated coding genes, although the altPEPs identified here probably correspond to only a fraction of the altPEPs actually present in plant cells.

To study the biological roles of altPEPs, two types of altPEPs were studied: one well conserved among Brassicaceae, and corresponding to the altORF18 of the DCL1 gene (FIG. 3c), and another, corresponding to the first altORF of the COI1 gene, but not conserved among Brassicaceae. A. thaliana seedlings were treated with the peptide AtDCL1alt18 (SEQ ID NO: 224) and root development analysed, since dcl mutants show longer main roots (Park, W., et al. Curr. Biol. 12, 1484-1495 (2002)). Interestingly, treatment with the peptide AtDCL1alt18 led to a decrease in root development (FIG. 2d). qRT-PCR and Western blot analysis to quantify DCL1 expression was performed and while no effect on mRNA expression was observed, application of the AtDCL1alt18 peptide increased DCL1 protein accumulation (FIG. 2e, f). Interestingly, treatment of plants with the peptide ΔtCOI1alt1 revealed an increase in COI1 protein levels, showing that this property is not restricted to DCL1 (FIG. 2g; FIG. 4a).

To extend this observation, bioinformatics was used to identify the first altPEPs of more than 10 amino acids in several well-known coding genes in A. thaliana. These were three genes: AP2 and EIN2, for which the first potential altPEP was conserved among the Brassicaceae (FIG. 3d, e), and CPK3, for which the first altPEP was not conserved. We also tested whether the altAUG1 of the EIN2 gene is translated in vitro, using an in vitro transcription/translation system in wheat germ extracts. To do this, the luciferase CDS was fused to the 5′UTR of EIN2 up to refAUG or altAUG1, and luciferase expression was compared. Interestingly, altAUG1 was shown to be effective in promoting translation (FIG. 3f). The translation efficiency was of the same order as the activity of refAUG, suggesting a similar amount of co-translation of the canonical EIN2 protein and altPEP. Plants were then treated with the three synthetic altPEPs (AtAP2alt1; SEQ ID NO: 243, AtCPK3alt1 and AtEIN2alt1; SEQ ID NO: 241). In each case, an increase in the reference protein was observed, without detecting any change in mRNA expression (FIG. 2g, h; FIG. 4b, c, d). This suggests that the increase in reference protein expression is probably a common property of altPEPs. In parallel, the conservation of altPEPs does not appear to be important for their activity. Based on these two findings, three altPEPs (SEQ ID NOs: 334 to 366) corresponding to different regions of the EIN2 gene were synthesised and A. thaliana plants were independently treated with them. Interestingly, all three peptides were able to increase the level of the EIN2 protein (FIG. 2h), showing that all the altPEPs tested share the property of increasing the levels of their reference protein.

Complementary Peptides or cPEP

It was demonstrated in the previous section that short peptides can physically interact with their nascent RNA (Lauressergues D, et al. Cell Reports. 38:110339, 2022). It was asked whether any peptide translated from a sequence located in a coding gene could share the same property (FIG. 5a). To this end, a complementary peptide of 10 amino acids (SEQ ID NO: 213) was designed corresponding to a 10 amino acid fragment of the luciferase protein, constitutively expressed in Arabidopsis thaliana (Whalley et al. Plant Cell. 23, 4079-95, 2011), and showing no homology with any sequence in the A. thaliana genome. Using RNA-IP followed by PCR on plants treated with HA-tagged peptides (cPEPluc-HA; SEQ ID NO: 387), it was validated that such a cPEP was specifically able to interact with luciferase mRNA, whereas a corresponding Scrambled peptide (SEQ ID NO: 386) could not (FIG. 5a). To determine whether this interaction might have biological relevance, plants were treated with synthetic cPEPluc and luciferase expression was analysed. While qPCR analysis showed no effect on mRNA abundance (FIG. 5b), treatment with the peptide increased luciferase activity compared with treatments with water or non-luciferase-specific peptides (FIG. 5c). A 96-well microplate assay was developed, wherein seeds were sown on MS solid half medium and 14-day-old seedlings were treated by adding 100 μL of liquid medium containing the peptides. In a second step, ten additional 10 amino acid peptides targeting distinct sequences of the luciferase gene were developed (SEQ ID NOs: 388 to 397), in all three frames (ORF1 corresponds to the canonical luciferase protein), and all were able to increase luciferase activity (FIG. 5d). In parallel, 7 cPEPs of 5 to 60 amino acids (SEQ ID NOs:398 to 403) were designed and synthesised. Exogenous treatment with these different peptides revealed an increase in luciferase activity for peptides ranging from 5 to 40 amino acids, while longer peptides had no effect (FIG. 5e). The effect of peptide concentration and treatment time on cPEPluc activity was then analysed. The optimum concentration of cPEP under these conditions was found to be 50 μM (FIG. 5f), and 24 hours of treatment showed the maximum effect of cPEP (FIG. 5g). Finally, to investigate whether cPEP could have side effects, A. thaliana plants expressing luciferase were treated with cPEPluc (SEQ ID NO: 213) or its Scrambled peptide (SEQ ID NO: 220) and transcriptomic and proteomic analysis was performed (FIG. 5h, i). Interestingly, no changes in the transcriptome and proteome of plants treated with cPEPluc were detected compared to plants treated with the Scrambled peptide, suggesting a high specificity of cPEPs for their target protein.

### Generalisation of the Concept of Peptides Increasing Protein Expression

In order to verify whether cPEPs can target any protein, the activity of 12 cPEPs targeting 12 different proteins for which antibodies were available was studied and validated in three different plant species. The question was whether the increase in the quantity of proteins observed after treatment with cPEP was sufficient to modulate plant development. To answer this question, development focused on different proteins in the model plant M. truncatula. Application of cPEPnsp1 (SEQ ID NO:164) decreased the amount of lateral roots (FIG. 6f, g), as did the nsp1 mutant and overexpression of NSP1, although both remained insensitive to the peptide (FIG. 6g). The M. truncatula Sickle (SKL) gene is homologous to AtEIN2, and skl mutants develop more nodules than wild-type plants (Penmetsa et al. Plant J. 55:580-595, 2008). Consistently, the application of a cPEPskl (SEQ ID NO: 411) led to a decrease in the amount of nodules (FIG. 6h, i). The RH10 protein of M. truncatula, which modulates the response of plants to a pathogenic oomycete, Aphanomyces euteiches, was then targeted (Camborde et al. New Phytol. 233:2232-2248. 2022). Treatment of plants with a cPEP targeting MtRH10 (SEQ ID NO: 412) increased plant resistance to the pathogen, as revealed by increased root development and decreased expression of A. euteiches α-tubulin in roots (FIG. 6j, k).

### Generalisation to Other Plant Models and Other Proteins

The work then focused on different A. thaliana proteins involved in different plant functions. Firstly, treatment of A. thaliana seedlings with a cPEPdcl1 (SEQ ID NO: 163) led to a reduction in primary root growth since dcl1 mutants have longer primary roots (Park et al. Curr. Biol. 12:1484-1495 (2002). (FIG. 7a, b). A. thaliana plants were then treated with cPEPs targeting regulators of chlorophyll content. Interestingly, a cPEP targeting the ABI5 protein (SEQ ID NO: 406) could be identified, decreasing chlorophyll content, and a cPEP targeting the SGR1 protein (SEQ ID NO: 407), increasing this content (FIG. 7c). Similarly, a cPEP targeting the HSP101 protein (SEQ ID NO: 404), involved in tolerance to heat stress (Queitsch et al, Plant Cell. 12:479-492, 2000), improved the viability of seedlings to heat shock (FIG. 7d). In parallel, several A. thaliana plant defence regulators were targeted, namely AGB1 (SEQ ID NO: 195), MAPKKK3 (SEQ ID NO: 187), JAR1 (SEQ ID NO: 192), MAPKKK5 (SEQ ID NO: 188), ABCG34 (SEQ ID NO: 194) and CPK3 (SEQ ID NO: 162). Interestingly, these cPEPs improved plant defence against the necrotrophic fungus Botrytis cinerea, as revealed by the decrease in lesion size observed in plants treated with the cPEPs compared with plants treated with an irrelevant peptide (FIG. 7e; FIG. 8). Next, several cPEPs were designed targeting different proteins involved in plant development by measuring the day of flowering. It was possible to identify cPEPs that increased plant development (SHY2 and MRB1, SEQ ID NOs: 178 and 180) while others were able to decrease plant development (BR11, SEQ ID NO: 198; BAK1, SEQ ID NO: 199; TAP46, SEQ ID NO: 181; SPT, SEQ ID NO: 182; EIN2, SEQ ID NO: 204; GA2OX7, SEQ ID NO: 183; PHYB, SEQ ID NO: 184; HAG5, SEQ ID NO: 185; SHR, SEQ ID NO: 186 and WUS, SEQ ID NO: 196) (FIG. 7f). Finally, it was investigated whether the cPEPs could have synergistic effects by mixing some of them. Interestingly, while each peptide separately decreased development by up to 17% (FIG. 7f), a mixture of cPEPs targeting EIN2, BR11, BAK1 and WUS (respectively SEQ ID NOs: 204, 198, 199 and 196), decreased development by 23%, as measured by flowering day (FIG. 7g, h) and leaf growth (FIG. 7i, j), showing a synergistic effect of the cPEPs. All these data showed that, in addition to improving protein expression, cPEPs are useful tools for precisely modulating several plant phenotypes.

cPEP Increases the Efficiency of Protein Translation

cPEPs increase protein quantity without disturbing mRNA levels, suggesting that cPEPs increase protein translation or stability. To distinguish between the two options, A. thaliana plants expressing the LUC gene were treated with cPEPluc (SEQ ID NO: 213) and cycloheximide (CHX), a translation inhibitor. The luciferase activity assay showed that the effect of cPEPluc was inhibited in the presence of CHX, demonstrating that cPEP does not affect protein stability (FIG. 9a). To support these data, the LUC gene was expressed with or without cPEPluc in the wheat germ in vitro transcription/translation system, where no protease activity occurs. This revealed that cPEPluc increased LUC activity in vitro, strongly supporting the idea that cPEPs increase protein translation efficiency (FIG. 9b).

Agronomic Benefits of cPEP

The main interest in cPEPs could be their use in agronomy to improve crop yields. To prove this, they were tested on plants of agronomic interest, focusing on the same phenotypes as those studied on the model plants. In this way, the work first focused on plant defence and the tomato JAR1 protein was targeted. In line with the previous observation in A. thaliana, treatment of tomato with cPEPjar1 (SEQ ID NO: 413) improved the plant's resistance to B. cinerea (FIG. 10a, b). In parallel, the homolog of HSP101 in soybean was identified and a cPEP targeting this protein was designed (SEQ ID NO: 408). Interestingly, treatment of soybean plants with this peptide increased their tolerance to heat stress (FIG. 10c, d). In parallel, it was validated in soybean that the use of cPEP can improve plant growth, using a mixture of cPEP targeting SHY2, MRB1 and SGR1 (respectively SEQ ID NOs: 410, 409 and 407) (FIG. 10e, f). Finally, it was tested whether cPEPs could reduce weed growth by targeting a Brassicaceae species, Barbarea vulgaris, and it was shown that a mixture of cPEPs targeting EIN2, BR11, BAK1 and WUS (respectively SEQ ID NOs: 417, 415, 414 and 416) was capable of doing so (FIG. 10g, h). To take this further, one of the most invasive and problematic weeds, Amaranthus, was selected and cPEPs were designed to target the corresponding proteins (SEQ ID NOs: 204, 176, 177 and 196 respectively). A mixture of these cPEPs was able to reduce plant growth (FIG. 10i, j).

### CONCLUSION

The results presented demonstrated the possibility of modulating the expression of any coding gene by external application of small synthetic peptides, thus facilitating the study of genes without the need for transgenic plants. This can be particularly relevant in the case of plants that are recalcitrant to genetic transformation. Simply watering or spraying plants with cPEPs enables a biological response in line with what is known about the function of the targeted proteins, such as modulating plant growth or improving plant resistance to certain pathogens. Agriculture in the 21st century faces a number of major challenges in feeding the world's growing population. Against this backdrop, there is an urgent need to find new molecules to maintain or improve crop yields. Until now, no credible alternative to chemical products has emerged. The use of CRISPR in agriculture is promising, particularly for improving crop growth, but it is difficult to think of weed control with this strategy. Despite its fantastic potential, the use of small RNAs faces the intractable problem of low penetration into plant cells, leading to low activity in the field, except for insect control. At the same time, it has been shown here that cPEPs are capable of modulating various plant traits, such as heat resistance or chlorophyll content, which are very difficult to manage with chemicals or other molecules.

In this context, the development of cPEP technology opens up a new avenue in agriculture with the use of small peptides. At the same time, the mode of action of cPEPs, based on the complementarity of their sequence with the targeted protein, will make it easier to identify non-specific interactions using bioinformatics, making it possible to target a single species, a family or all plants.

Finally, as peptides are short polymers of amino acids, they are likely to be rapidly degraded by the soil microbiota, unlike chemical pollutants. Furthermore, it seems difficult for peptides to penetrate animal cells without the presence of cell-penetrating peptides, which suggests that cPEPs will have no biological activity in animals or humans, apart from possible intrinsic toxicity.

1-11. (canceled)

12. A composition comprising a cPEP as an active substance, said cPEP:
having a size from 4 to 70 amino acids, the amino acid sequence of which corresponds to the translation via the genetic code of a fragment of a nucleic acid sequence naturally translated on an mRNA in a plant cell which corresponds to the open reading frame of a protein encoded by said mRNA; and
being capable of modulating the accumulation of said protein in the plant cell and not being capable of modulating the accumulation of the mRNA encoding said protein.

13. The composition according to claim 12, said cPEP having a size from 4 to 41 amino acids.

14. The composition according to claim 12, said protein being encoded by an ORF comprising a nucleic acid sequence having at least 80% identity with a sequence chosen from the sequences: SEQ ID NO: 1 (ORF of the Aae15 protein, A. thaliana), SEQ ID NO: 2 (ORF of the Aae16 protein, A. thaliana), SEQ ID NO: 3 (ORF of the abcg11 protein, A. thaliana), SEQ ID NO: 4 (ORF of the Abdcg34 protein, A. thaliana), SEQ ID NO: 5 (ORF of the Acc1 protein, A. thaliana), SEQ ID NO: 6 (ORF of the Agb1 protein, A. thaliana), SEQ ID NO: 7 (ORF of the Als protein, A. thaliana), SEQ ID NO: 8 (ORF of the Anac076 protein, A. thaliana), SEQ ID NO: 9 (ORF of the Apg9 protein, A. thaliana), SEQ ID NO: 10 (ORF of the Arlb1 protein, A. thaliana), SEQ ID NO: 11 (ORF of Arr1 protein, A. thaliana), SEQ ID NO: 12 (ORF of Arr5 protein, A. thaliana), SEQ ID NO: 13 (ORF of Arr6 protein, A. thaliana), SEQ ID NO: 14 (ORF of At59 protein, A. thaliana), SEQ ID NO: 15 (ORF of Bak1 protein, A. thaliana), SEQ ID NO: 16 (ORF of the Bccp1 protein, A. thaliana), SEQ ID NO: 17 (ORF of the Bccp2 protein, A. thaliana), SEQ ID NO: 18 (ORF of the Bri1 protein, A. thaliana), SEQ ID NO: 19 (ORF of the Bzo2 h3 protein, A. thaliana), SEQ ID NO: 20 (ORF of the Cesa6 protein, A. thaliana), SEQ ID NO: 21 (ORF of the Cipk3 protein, A. thaliana), SEQ ID NO: 22 (ORF of the Cks1 protein, A. thaliana), SEQ ID NO: 23 (ORF of the Cobl8 protein, A. thaliana), SEQ ID NO: 24 (ORF of Coi1 protein, A. thaliana), SEQ ID NO: 25 (ORF of Coi1 protein, A. thaliana), SEQ ID NO: 26 (ORF of Cpk3 protein, A. thaliana), SEQ ID NO: 27 (ORF of Cpk3 protein, A. hypochondriacus), SEQ ID NO: 28 (ORF of Cpk3 protein, B. distachyon), SEQ ID NO: 29 (ORF of Cpk3 protein, B. distachyon), SEQ ID NO: 30 (ORF of Cpk3 protein, G. max), SEQ ID NO: 31 (ORF of Cpk3 protein, G. max), SEQ ID NO: 32 (ORF of Cpk3 protein, G. max), SEQ ID NO: 33 (ORF of Cpk3 protein, G. max), SEQ ID NO: 34 (ORF of Cpk3 protein, O. sativa), SEQ ID NO: 35 (ORF of Cpk3 protein, O. sativa), SEQ ID NO: 36 (ORF of Cpk3 protein, S. lycopersicum), SEQ ID NO: 37 (ORF of Cpk3 protein, Z. mays), SEQ ID NO: 38 (ORF of Cpk3 protein, Z. mays), SEQ ID NO: 39 (ORF of Cpk3 protein, Z. mays), SEQ ID NO: 40 (ORF of Cpk3 protein, B. rapa), SEQ ID NO: 41 (ORF of Cpk3 protein, B. rapa), SEQ ID NO: 42 (ORF of Cpk3 protein, H. vulgare), SEQ ID NO: 43 (ORF of Cpk3 protein, H. vulgare), SEQ ID NO: 44 (ORF of Cpk3 protein, S. tuberosum), SEQ ID NO: 45 (ORF of Cpk3 protein, A. palmeri), SEQ ID NO: 46 (ORF of Cpk3 protein, M. truncatula), SEQ ID NO: 47 (ORF of Cpk3 protein, M. truncatula), SEQ ID NO: 48 (ORF of Cpk3 protein, T. aestivum), SEQ ID NO: 49 (ORF of Cpk3 protein, T. aestivum), SEQ ID NO: 50 (ORF of Cpk3 protein, T. aestivum), SEQ ID NO: 51 (ORF of Cpk3 protein, T. aestivum), SEQ ID NO: 52 (ORF of Cpk3 protein, L. perenne), SEQ ID NO: 53 (ORF of Cpk3 protein, L. perenne), SEQ ID NO: 54 (ORF of Cpk3 protein, L. perenne), SEQ ID NO: 55 (ORF of protein Cpk3, L. perenne), SEQ ID NO: 56 (ORF of protein Crk34, A. thaliana), SEQ ID NO: 57 (ORF of protein Cyp705a18, A. thaliana), SEQ ID NO: 58 (ORF of protein Cyp71b26, A. thaliana), SEQ ID NO: 59 (ORF of protein Cyp78a8, A. thaliana), SEQ ID NO: 60 (ORF of Cyp97b3 protein, A. thaliana), SEQ ID NO: 61 (ORF of Dcl1 protein, A. thaliana), SEQ ID NO: 62 (ORF of Dcl1 protein, A. thaliana), SEQ ID NO: 63 (ORF of Dcl1 protein, A. hypochondriacus), SEQ ID NO: 64 (ORF of Dcl1 protein, B. distachyon), SEQ ID NO: 65 (ORF of Dcl1 protein, G. max), SEQ ID NO: 66 (ORF of Dcl1 protein, G. max), SEQ ID NO: 67 (ORF of Dcl1 protein, O. sativa), SEQ ID NO: 68 (ORF of Dcl1 protein, S. lycopersicum), SEQ ID NO: 69 (ORF of Dcl1 protein, Z. mays), SEQ ID NO: 70 (ORF of Dcl1 protein, B. rapa), SEQ ID NO: 71 (ORF of Dcl1 protein, H. vulgare), SEQ ID NO: 72 (ORF of Dcl1 protein, S. tuberosum), SEQ ID NO: 73 (ORF of Dcl1 protein, M. truncatula), SEQ ID NO: 74 (ORF of Dcl1 protein, T. aestivum), SEQ ID NO: 75 (ORF of Dcl1 protein, T. aestivum), SEQ ID NO: 76 (ORF of Dcl1 protein, T. aestivum), SEQ ID NO: 77 (ORF of Dcl1 protein, T. aestivum), SEQ ID NO: 78 (ORF of Dcl1 protein, L. perenne), SEQ ID NO: 79 (ORF of the Dcl1 protein, L. perenne), SEQ ID NO: 80 (ORF of the Dur3 protein, A. thaliana), SEQ ID NO: 81 (ORF of the Ein2 protein, A. thaliana), SEQ ID NO: 82 (ORF of Emb175 protein, A. thaliana), SEQ ID NO: 83 (ORF of Emb2726 protein, A. thaliana), SEQ ID NO: 84 (ORF of Emb9 protein, A. thaliana), SEQ ID NO: 85 (ORF of Epsps protein, A. thaliana), SEQ ID NO: 86 (ORF of Fnr1 protein, A. thaliana), SEQ ID NO: 87 (ORF of the Fve protein, A. thaliana), SEQ ID NO: 88 (ORF of the Ga2ox7 protein, A. thaliana), SEQ ID NO: 89 (ORF of the Gapc protein, N. benthamiana), SEQ ID NO: 90 (ORF of the Gcn2 protein, A. thaliana), SEQ ID NO: 91 (ORF of the Gdi2 protein, A. thaliana), SEQ ID NO: 92 (ORF of Gln2 protein, A. thaliana), SEQ ID NO: 93 (ORF of Gsl3 protein, A. thaliana), SEQ ID NO: 94 (ORF of Hag5 protein, A. thaliana), SEQ ID NO: 95 (ORF of the Hda18 protein, A. thaliana), SEQ ID NO: 96 (ORF of the Hexo1 protein, A. thaliana), SEQ ID NO: 97 (ORF of the Hppd protein, A. thaliana), SEQ ID NO: 98 (ORF of the Hsl1 protein, A. thaliana), SEQ ID NO: 99 (ORF of the Iaa31 protein, A. thaliana), SEQ ID NO: 100 (ORF of the Iqd28 protein, A. thaliana), SEQ ID NO: 101 (ORF of the Jac1 protein, A. thaliana), SEQ ID NO: 102 (ORF of the Jar1 protein, A. thaliana), SEQ ID NO: 103 (ORF of the Kp1 protein, A. thaliana), SEQ ID NO: 104 (ORF of the Lrx2 protein, A. thaliana), SEQ ID NO: 105 (ORF of the Mapkkk3 protein, A. thaliana), SEQ ID NO: 106 (ORF of the Mapkkk5 protein, A. thaliana), SEQ ID NO: 107 (ORF of the Mfp2 protein, A. thaliana), SEQ ID NO: 108 (ORF of the Mrb1 protein, A. thaliana), SEQ ID NO: 109 (ORF of the Nsp1 protein, M. truncatula), SEQ ID NO: 110 (ORF of the Nsp1 protein, A. thaliana), SEQ ID NO: 111 (ORF of the Nsp1 protein, B. distachyon), SEQ ID NO: 112 (ORF of the Nsp1 protein, G. max), SEQ ID NO: 113 (ORF of the Nsp1 protein, G. max), SEQ ID NO: 114 (ORF of the Nsp1 protein, O. sativa), SEQ ID NO: 115 (ORF of the Nsp1 protein, S. lycopersicum), SEQ ID NO: 116 (ORF of the Nsp1 protein, S. lycopersicum), SEQ ID NO: 117 (ORF of the Nsp1 protein, Z. mays), SEQ ID NO: 118 (ORF of the Nsp1 protein, Z. mays), SEQ ID NO: 119 (ORF of Nsp1 protein, Z. mays), SEQ ID NO: 120 (ORF of Nsp1 protein, Z. mays), SEQ ID NO: 121 (ORF of Nsp1 protein, B. rapa), SEQ ID NO: 122 (ORF of Nsp1 protein, H. vulgare), SEQ ID NO: 123 (ORF of Nsp1 protein, H. vulgare), SEQ ID NO: 124 (ORF of Nsp1 protein, H. vulgare), SEQ ID NO: 125 (ORF of the Nsp1 protein, H. vulgare), SEQ ID NO: 126 (ORF of the Nsp1 protein, H. vulgare), SEQ ID NO: 127 (ORF of the Nsp1 protein, H. vulgare), SEQ ID NO: 128 (ORF of the Nsp1 protein, H. vulgare), SEQ ID NO: 129 (ORF of the Nsp1 protein, S. tuberosum), SEQ ID NO: 130 (ORF of the Nsp1 protein, S. tuberosum), SEQ ID NO: 131 (ORF of the Nsp1 protein, T. aestivum), SEQ ID NO: 132 (ORF of the Nsp1 protein, T. aestivum), SEQ ID NO: 133 (ORF of the Nsp1 protein, L. perenne), SEQ ID NO: 134 (ORF of the Nsp1 protein, L. perenne), SEQ ID NO: 135 (ORF of the Pds protein, A. thaliana), SEQ ID NO: 136 (ORF of the Pen3 protein, A. thaliana), SEQ ID NO: 137 (ORF of the Phyb protein, A. thaliana), SEQ ID NO: 138 (ORF of the Pif3 protein, A. thaliana), SEQ ID NO: 139 (ORF of the Pizza protein, A. thaliana), SEQ ID NO: 140 (ORF of the Ppox1 protein, A. thaliana), SEQ ID NO: 141 (ORF of the Ppox2 protein, A. thaliana), SEQ ID NO: 142 (ORF of the Prp39 protein, A. thaliana), SEQ ID NO: 143 (ORF of the PsbA protein, A. thaliana), SEQ ID NO: 144 (ORF of the Pskr1 protein, A. thaliana), SEQ ID NO: 145 (ORF of the Rd21 protein, A. thaliana), SEQ ID NO: 146 (ORF of the Ring1 protein, A. thaliana), SEQ ID NO: 147 (ORF of the Rosa protein, A. thaliana), SEQ ID NO: 148 (ORF of the Rpt4a protein, A. thaliana), SEQ ID NO: 149 (ORF of the Sfr6 protein, A. thaliana), SEQ ID NO: 150 (ORF of the Shr protein, A. thaliana), SEQ ID NO: 151 (ORF of the Shy2 protein, A. thaliana), SEQ ID NO: 152 (ORF of the Skl protein, M. truncatula), SEQ ID NO: 153 (ORF of the Sps1 protein, A. thaliana), SEQ ID NO: 154 (ORF of the Spt protein, A. thaliana), SEQ ID NO: 155 (ORF of the Stn8 protein, A. thaliana), SEQ ID NO: 156 (ORF of the Tap46 protein, A. thaliana), SEQ ID NO: 157 (ORF of the Topp6 protein, A. thaliana), SEQ ID NO: 158 (ORF of the TubB6 protein, A. thaliana), SEQ ID NO: 159 (ORF of the TubB8 protein, A. thaliana), SEQ ID NO: 160 (ORF of the Uba1a protein, A. thaliana), SEQ ID NO: 161 (ORF of the Vim3 protein, A. thaliana), SEQ ID NO: 381 (ORF of the Sgr1 protein, A. thaliana), SEQ ID NO: 382 (ORF of the Abi5 protein, A. thaliana), SEQ ID NO: 383 (ORF of the Hsp101 protein, A. thaliana), SEQ ID NO: 384 (ORF of the Rh10 protein, M. truncatula) and SEQ ID NO: 385 (ORF of the Wus protein, A. thaliana).

15. The composition according to claim 12, wherein the sequence of said cPEP is chosen from the sequences: SEQ ID NO: 162 (cPEPcpk3), SEQ ID NO: 163 (cPEPdcl1), SEQ ID NO: 164 (cPEPnsp1_3), SEQ ID NO: 165 (cPEPnsp1_1), SEQ ID NO: 166 (cPEPnsp1_2), SEQ ID NO: 167 (cPEPnsp1_4), SEQ ID NO: 168 (cPEPnsp1_5), SEQ ID NO: 169 (cPEPnsp1 5aa), SEQ ID NO: 170 (cPEPnsp1 20aa), SEQ ID NO: 171 (cPEPnsp1 30aa), SEQ ID NO: 172 (cPEPnsp1 40aa), SEQ ID NO: 173 (cPEPnsp1 60aa), SEQ ID NO: 174 (cPEPnsp1 80aa), SEQ ID NO: 175 (cPEPgapc), SEQ ID NO: 176 (cPEPbri1), SEQ ID NO: 177 (cPEPbak1), SEQ ID NO: 178 (cPEPshy2), SEQ ID NO: 179 (cPEPpizza), SEQ ID NO: 180 (cPEPmrb1), SEQ ID NO: 181 (cPEPtap46), SEQ ID NO: 182 (cPEPspt), SEQ ID NO: 183 (cPEPga2ox7), SEQ ID NO: 184 (cPEPphyb), SEQ ID NO: 185 (cPEPhag5), SEQ ID NO: 186 (cPEPshr), SEQ ID NO: 187 (cPEPmapkkk3), SEQ ID NO: 188 (cPEPmapkkk5_1), SEQ ID NO: 189 (cPEPmapkkk5_2), SEQ ID NO: 190 (cPEPring1), SEQ ID NO: 191 (cPEPros1), SEQ ID NO: 192 (cPEPjar1), SEQ ID NO: 193 (cPEPcoi1), SEQ ID NO: 194 (cPEPabcg34), SEQ ID NO: 195 (cPEPagb1), SEQ ID NO: 196 (cPEPwus), SEQ ID NO: 197 (AhEIN2), SEQ ID NO: 198 (AhBRI1), SEQ ID NO: 199 (AhBAK1), SEQ ID NO: 200 (AtEIN2cPEP1), SEQ ID NO: 201 (AtEIN2cPEP2), SEQ ID NO: 202 (AtEIN2cPEP3), SEQ ID NO: 203 (AtEIN2cPEP13), SEQ ID NO: 204 (cPEPein2_1), SEQ ID NO: 205 (cPEPein2_2), SEQ ID NO: 206 (cPEPein2_3), SEQ ID NO: 404 (cPEPhsp101), SEQ ID NO: 406 (cPEPabi5), SEQ ID NO: 407 (cPEPsgr1), SEQ ID NO: 408 (cPEPhsp101), SEQ ID NO: 409 (cPEPmrb1), SEQ ID NO: 410 (cPEPshy2), SEQ ID NO: 411 (cPEPsk1), SEQ ID NO: 412 (cPEPrh10), SEQ ID NO: 413 (cPEpjar1), SEQ ID NO: 414 (cPEPbak1), SEQ ID NO: 415 (cPEPbri1), SEQ ID NO: 416 (cPEPwus) and SEQ ID NO: 417 (cPEPein2).

16. The composition according to claim 12, said cPEP being at a concentration from 5 μM to 500 μM or from 30 μM to 70 μM, or at a concentration of 50 μM.

17. The composition according to claim 12, said composition being a phytopharmaceutical composition, a herbicidal composition or a coating composition.

18. The composition according to claim 12, said composition being a coating composition further comprising at least one fixing agent.

19. The composition according to claim 12, said composition being a herbicidal to decrease the growth of weeds by targeting a Amaranthus and Brassicaceae species.

20. A method of modulating accumulation of a protein in a plant cell, comprising introducing to said plant cell a cPEP as a plant protection agent,
said cPEP having a size from 4 to 70 amino acids, the amino acid sequence of which corresponds to the translation via the genetic code of a fragment of a nucleic acid sequence naturally translated on an mRNA in a plant cell which corresponds to the open reading frame of said protein encoded by said mRNA,
said cPEP being capable of modulating the accumulation of said protein in the plant cell and not being capable of modulating the accumulation of the mRNA encoding said protein.

21. The method according to claim 20, said cPEP having a size from 4 to 41 amino acids.

22. The method according to claim 20, said protein being encoded by an ORF comprising a nucleic acid sequence having at least 80% identity with a sequence chosen from the sequences: SEQ ID NOs: 1 to 161 and 381 to 385.

23. The method according to claim 20, wherein the sequence of said cPEP is chosen from the sequences: SEQ ID NOs: 162 to 206, 404 and 406 to 417.

24. A method of modulating the accumulation of a protein in a plant cell comprising a step for introducing:
a cPEP; or
of a nucleic acid encoding said cPEP and the means of expressing it,
in said plant cell, the introduction of said cPEP resulting in a modulation of the quantity of said protein in said plant cell,
said cPEP having a size from 4 to 70 amino acids, the amino acid sequence of which corresponds to the translation via the genetic code of a fragment of a nucleic acid sequence naturally translated on an mRNA in a plant cell which corresponds to the open reading frame of said protein encoded by said mRNA,
said cPEP being capable of modulating the accumulation of said protein in the plant cell and not being capable of modulating the accumulation of the mRNA encoding said protein.

25. The method according to claim 24, said cPEP having a size from 4 to 41 amino acids.

26. The method according to claim 24, said protein being encoded by an ORF comprising a nucleic acid sequence having at least 80% identity with a sequence chosen from the sequences: SEQ ID NOs: 1 to 161 and 381 to 385.

27. The method according to claim 24, wherein the sequence of said cPEP is chosen from the sequences: SEQ ID NOs: 162 to 206, 404 and 406 to 417.

28. The method according to claim 24, said method:
promoting the development of a plant; or
slowing down or prevent the development of a plant, or
promoting the stress tolerance of a plant.

29. The method according to claim 24, said method modulating the accumulation of a protein in a plant cell chosen from: Alopecurus myosuroides, Amaranthus hypochondriacus, Amaranthus palmeri, Amaranthus tuberculatus, Arabidopsis halleri, Arabidopsis lyrata, Arabidopsis thaliana, Barbarea vulgaris, Boechera stricta, Brachypodium distachyon, Brassica napus (oilseed rape), Brassica oleracea, Brassica rapa (oilseed rape), Camelina sativa, Capsella grandiflora, Capsella rubella, Carica papaya, Eutrema salsugineum, Glycine max (soya), Gossypium raimondii, Gossypium spp. (cotton), Hordeum vulgare (barley), Lollium spp, Lotus japonicus (birdsfoot trefoil), Medicago sativa (alfalfa), Medicago truncatula (lucerne), Nicotiana benthamiana (tobacco), Oryza sativa (rice), Pisum sativum (peas), Raphanus sativus, Solanum lycopersicum (tomato), Solanum melongena (aubergine), Solanum tuberosum (potato), Thellungiella halophila, Theobroma cacao, Triticum spp. (wheat), Vitis vinifera (grapevine) and Zea mays (maize).

30. The method according to claim 24, said method modulating the accumulation of a protein in a plant cell chosen from: Brassica napus, Brassica oleracea, Brassica rapa, Camelina sativa, Carica papaya, Glycine max, Gossypium raimondii, Gossypium spp., Hordeum vulgare, Lollium spp., Lotus japonicus, Medicago sativa, Medicago truncatula, Nicotiana benthamiana, Oryza sativa, Pisum sativum, Raphanus sativus, Solanum lycopersicum, Solanum melongena, Solanum tuberosum, Theobroma cacao, Triticum spp., Vitis vinifera and Zea mays.


SEQUENCE LISTING (418 sequences)

SEQ ID NO: 1 - DNA, 2184 bp, Arabidopsis thaliana
  misc_feature 1..2184: note = ORF of protein Aae15
  source 1..2184: mol_type = unassigned DNA
atgcaaattcgtctgaaacctgattattccttcttcatcgcctcttcctcaacgatggcttcgacttcttctttgggaccttctacactactctcttacggttctccttctcgtcagtttcctgattttgggttcagattgatttcgggtcacgaaagtgttcgaattccatcattccggcgatttcgggttcactgcgagtcaaaggaaaaagaagtgaagccgtcttctccatttcttgaaagctcctcgttttcgggagatgctgctttgcgctctagtgaatggaaggctgttcctgatatttggagatcatctgcagaaaagtatggtgatagagttgcattggtggatccttatcatgatcctcctttgaaactgacgtacaagcagttggaacaagaaattttggactttgctgagggcttacgagttcttggagtgaaagcagatgagaagattgcactttttgctgataactcctgccgatggcttgtttcagatcaaggtataatggccacaggggcagtcaatgttgtcagaggatctaggtcctctgttgaagagttactgcagatataccgtcattctgaaagcgtagccattgttgtggataatcctgagtttttcaaccgcattgctgagtcatttacgtcaaaggcatctctgagatttttgatacttctctggggtgagaaatcatcactggtcacacaggggatgcagattccagtttacagttatgcagaaattataaaccaaggacaggagagtcgtgcaaaattatcagcatctaatgataccaggagctatagaaatcaattcatcgattcagatgatacagctgcaattatgtataccagtggtaccacgggaaatccaaaaggcgttatgcttacacatcggaatctcttacaccagataaaacatttatccaaatatgtacctgctcaagctggggataaatttctaagcatgctaccatcatggcatgcctatgaacgtgctagtgaatacttcatattcacttgtggagttgagcaaatgtatacatctataagatacttaaaggatgatctaaagcggtaccaaccgaactatattgtgtccgttcctctagtatatgagacactttacagtgggattcaaaagcaaatttctgcaagttctgctggccgtaaatttctagcacttacattgatcaaagtcagtatggcatatatggagatgaaaaggatatatgagggtatgtgtctgacaaaagagcaaaagcctccaatgtatattgttgcttttgtggattggttgtgggcgagagtaattgctgccttgttgtggccattacatatgttggccaaaaagcttatctacaagaaaattcattcgtctattgggatatcgaaggctggtattagcggaggtggtagtttacccattcatgttgacaagttttttgaggccatcggtgtgattctacaaaatggttatggtttgacagagacctcacctgttgtctgtgcacggacacttagctgcaatgttcttggctcagctgggcatccaatgcatggtacagaattcaaaattgtagatcctgagactaataatgtactccctcctggttcaaagggcattatcaaagtcagaggtccacaggttatgaagggttattataagaatccatcgactacaaagcaagttctaaatgagagtggatggttcaatacaggagacaccggttggattgctcctcatcactcaaaagggcggagtcgccactgtggaggtgtcattgttcttgaaggccgtgcaaaagacacaattgtactttccacaggtgaaaatgtggaaccgttggagattgaagaagccgccatgagaagcagggtgattgaacaaattgttgttattggacaggaccgacgtcgccttggagctatcattatcccaaacaaagaggaagcacaaagagtagatcctgaaacatccaaagaaacactaaagagcttggtctaccaagaactgagaaaatggacatcagaatgttcgtttcaagtcggaccagtcttgatcgtcgacgaccctttcacgatagacaacgggttaatgacaccaactatgaagattagacgggacatggtcgtggctaaatacaaagaggagattgatcaactctacagttaa

SEQ ID NO: 2 - DNA, 2169 bp, Arabidopsis thaliana
  misc_feature 1..2169: note = ORF of protein Aae16
  source 1..2169: mol_type = unassigned DNA
atggcttcaacgtctctcggagcttcgattctcgtttctcactgctcctcagctcctgaatttcaagtttctgggatgagattggtgtttggttacaaggcttttggctgcagaacttcacgcaggggatttcgagttcgctgcgaatccaagattcaggagaaggagttaaggcggtgttcgccattcttagaacgcttatcattgccaagggaggctgctttgagctctaatgaatggaagtctgttcctgatatttggagatcatctgtggagaagtacggtgacagagttgcggtggtagatccgtatcatgacccgccttctacattcacgtacagacagttggaacaagaaatcttggactttgttgagggtttacgagtcgttggagtgaaagcagacgagaagattgcactttttgctgataactcctgtcgatggcttgttgcggatcaaggtataatggccacaggagcagtcaatgttgttagaggatccagatcgtctgtggaagagttattgcagatatactgtcattcggaaagtgtagcccttgttgtggataaccctgagtttttcaatcgcattgcggagtcattttcttacaaggcagctccaaaatttgtgattcttctctggggggaaaaatcgtcgttggttacagcgggtaggcacacaccagtctatagttacaacgaaattaaaaagtttggacaagagagacgtgcaaaatttgcaagatctaatgattctgggaagtatgaatatgaatacatcgatccagatgatatagccacaattatgtataccagtggaaccacaggaaatccaaaaggtgttatgctcacacatcagaatttgttacaccagataagaaacttgtccgattttgtgcctgcggaagctggggaaagatttctgagtatgttgccatcatggcatgcttatgaacgggcttgtgaatacttcatatttacatgtggagttgagcaaaagtatacgtctataagattcttaaaggatgatctcaagcgttatcaaccacactatcttatttcagttcctttagtatatgagacactctacagcgggattcaaaagcaaatttctgcaagctcccctgctcgtaaatttttggcacttacattgatcaaagtcagcctggcatatacggaaatgaaaagagtttatgagggtctttgtttgacaaaaaatcaaaagcctccaatgtatattgtttcgttggtggattggttgtgggcgagagtagttgcatttttcctatggccattgcatatgttggctgaaaagcttgtacacagaaaaattcgttcgtctattgggataacaaaggctggtgttagtggaggtggtagtttacctatgcatgttgacaagttttttgaggccatcggtgtgaatgtacaaaatgggtatggtttgacagaaacctcaccggttgtctctgcgcggaggcttaggtgtaacgttcttggctcagttgggcatcctattaaagatacagagttcaaaattgtagatcatgagactggtactgttcttccacctggttcaaagggcattgtcaaagtcagaggcccaccggtgatgaaaggttactacaagaatccactggccaccaagcaggttatagatgacgatggatggttcaatactggagatatgggttggattactcctcagcactcaacaggacggagtcgtagctgtggaggtgtcatcgttcttgaagggcgtgccaaggacactatcgtgctttccacaggtgaaaatgtggaaccattggagattgaagaagcggccatgagaagcaatttgattcaacaaatagtggttattggacaggatcaacgccgccttggagctattgttatcccaaacaaagaagcagcagaaggagcagcaaagcagaaaatttcacctgtagattctgaagtcaatgaacttagcaaggagacgataacaagcatggtctatgaagaactaaggaaatggacgtcacaatgctcgttccaagttggaccagttctgatcgtggatgaaccattcacgatagacaacggtttaatgacaccgacaatgaaaataagacgggacaaggtggttgatcaatacaagaatgaaatagaaagactctacaagtag

SEQ ID NO: 3 - DNA, 2112 bp, Arabidopsis thaliana
  misc_feature 1..2112: note = ORF of protein Abcg11
  source 1..2112: mol_type = unassigned DNA
atggagatagaagcaagcagacaacaaaccaccgtaccggtttcagtcggcggtgggaattttccggttggtgggttaagtccgttgagtgaagctatatggagagagaaagctccaacggagtttgtcggagacgtgtcagcgaggcttacgtggcaagatctgacggtgatggtgactatgggagatggtgagactcaaaatgttcttgaaggtcttaccggttacgctgaaccgggatctctcacggctctcatgggcccttccggctccgggaaatctactatgctcgacgcattagctagccgcctcgctgctaacgcttttctctccggcaccgttcttcttaacggccgcaaaaccaaactctcctttggaacagctgcatatgtgacccaagacgataacttgattggtacgttaactgtgagagagacgatttggtattcagcgagagttcgtcttccggacaagatgttacggtcggagaagcgtgctctagtggaaaggacaataatagagatgggtcttcaggattgtgcagatactgtcataggaaattggcatttgcgtggaattagtggtggagagaagagaagagtcagtattgctcttgagattttaatgagacctcgtttgctgtttctcgatgagccaacaagtggtcttgatagtgcttctgctttctttgtgactcagactttgcgtgctttatcacgagatggaaggacggtgatagcatcgatccaccaaccgagcagtgaggtttttgagttgtttgatcggttgtatctgctttctggaggcaaaactgtttactttggtcaagcttcagatgcatatgagttctttgcacaagctgggtttccatgtcctgcgttaaggaacccttctgatcatttcctgagatgcataaactcagactttgacaaagttagagccactttgaaaggttctatgaagctaaggtttgaagcaagcgatgatccgttggaaaagattaccacagccgaagctattagactattggttgactattaccacacttctgattactactacacagcaaaggctaaggttgaggagatatcccaatttaaagggactattcttgactccggaggcagccaggctagtttccttctgcagacctacactttaacaaagcgatcattcatcaacatgtcaagggactttggttattactggctcagacttctaatttacatcttggtcacagtctgcattggaaccatctatctgaacgttgggactagctacagcgccattctggcacggggctcatgtgcgtctttcgtctttggctttgtaacattcatgtcaattggtggatttccttcgttcgttgaagacatgaaggtcttccaaagagagagactaaacgggcactatggagtagctgcgtttgtgatagccaacacattatctgcaacaccattcctgatcatgataactttcatctccgggacaatctgttacttcatggtgggtctgcatccaggattcactcactacctcttctttgttctctgcctctatgcaagtgtcaccgttgtggagagcttaatgatggcaattgctagtattgttcccaacttcctcatgggtatcatcatcggagctggaatccaggggatcttcatgctggtttctggattcttcaggcttccaaatgacatcccaaaacctttctggcgttacccaatgtcctacatcagcttccacttctgggctctacaaggtcagtaccagaatgatctgagaggcttgacgtttgacagccaagggagtgctttcaagattccgggtgaatatgttcttgagaacgtctttcagatcgacttgcatcgatcaaaatggattaacctcagtgtgattcttagcatgattatcatctaccgcatcatcttcttcatcatgattaagaccaacgaggatgttactccttgggtcagaggttacatagcacgcagaaggatgaagcagaagaatggaactcagaacaccacagttgcaccagatggtcttacccagtctccttctctgagaaattacattgctacacgaacagatggagctcgcagatggtag

SEQ ID NO: 4 - DNA, 4362 bp, Arabidopsis thaliana
  misc_feature 1..4362: note = ORF of protein Abdcg34
  source 1..4362: mol_type = unassigned DNA
atgttaggacgagatgaagatctggtccgaacaatgagcgggagaggaagtttaggctccacaagtcaccggagtttagccggagctgcttcaaaaagcttcagagatgtgtttgctcccccaacggacgacgtgtttggtcggagcgatagacgagaagaagatgacgtggagctccgatgggcggcgcttgagagattaccgacttacgatcggttaagaaaaggtatgttgccgcaaacgatggtcaacggtaagattggacttgaagatgttgacgtcacgaatcttgctcctaaggagaagaaacatcttatggaaatgattctgaaattcgttgaagaagataacgaaaagtttcttcggcgattgagagaaagaactgacagagtgggaatcgaagttccgaaaattgaagtaaggtatgagaatctttcagtggaaggagatgttcgtagtgcaagcagagctcttcctactctcttcaacgtcactttgaatacaatcgagagtattcttggattattccacctgcttccatctaagaagaggaagattgagattcttaaagatataagtggcattatcaaaccatcaaggatgactttattacttggtccacctagttcagggaaaacaactttgttacaagctttagctgggaagcttgatgacactctccagatgtctgggaggataacttactgcggtcatgagtttcgtgagtttgttcctcagaagacttgtgcttacattagtcagcatgaccttcactttggagaaatgactgtgcgagagtcattggacttttcgggacgatgtttaggtgttgggactcggtaccagcttttgactgagctctcaaggagggagagagaagctggaattaagcctgaccctgagattgatgcatttatgaaatccattgctatatctggccaagaaactagtttggttacagactatgtacttaagttacttggtctggacatttgtgctgacacacttgttggagatgtgatgagacgaggtatttctggtggacagcggaaacgtctaacaacaggagagatgttggttggaccagcaacagctcttttcatggatgaaatatcaacagggttagacagttccacaacattccaaatttgcaagttcatgaggcaactagttcatatcgcggatgtcacaatggtcatttcgcttctacaacctgcgccagagacatttgagcttttcgacgacattatcctactctcagagggccaaattgtctaccaggggtcacgagacaacgttcttgagttctttgagtacatgggtttcaaatgtcctgaaaggaaaggtattgcagattttctgcaagaagttacgtctaagaaggaccaagaacagtattggaacaggagagaacatccttacagctatgtatcagtgcatgacttctcaagcggcttcaattcttttcacgcagggcaacaacttgcttcagaattcagggttccttatgacaaagcaaaaactcatcctgcagcactagtgacacaaaagtatggtatatcaaacaaggatctattcaaggcatgctttgatagagaatggcttcttatgaaacgtaactcctttgtgtacgtgttcaagaccgtccagataaccatcatgtctttgattgccatgacggtttattttcggacagaaatgcatgtcggcactgtgcaagatggtcaaaagttttatggtgctctctttttcagcttgatcaatctaatgtttaatggaatggctgaactagccttcacagtgatgaggcttccggttttcttcaagcagagggacttcttgttctatcctccatgggctttcgcattacccggttttcttctcaagattccgttatctcttattgaatcagttatatggatcgctcttacatactacactattggttttgctccttctgctgccaggttcttccggcagttgctagcatacttctgtgtgaatcagatggcactttctttatttagattccttggagcccttggaagaacagaagtcatcgccaactctggcgggacattagcattgctagtcgtatttgttcttggaggctttattatttctaaagatgacatcccgtcgtggctgacttggtgctattatacatcacctatgatgtatggacagactgcattagttataaatgaatttttggatgagcgatggggcagcccaaacaatgatacacgcatcaacgcgaaaacagttggagaagtcttgctgaagagcagaggcttctttacagaaccatattggttttggatctgtattggggcgctacttgggtttactgtgttgttcaacttctgttacataatagccttgatgtatttgaaccctcttggtaactccaaagctactacagttgtggaagaaggtaaagacaaacacaaagggagtcacagtggaacaggaggttctgtagtggaactcactagtacttcaagtcatggaccaaagaaaggaatggttttacctttccaaccactttctctagcattcaacaatgtgaactactacgtggatatgcctgcagaaatgaaggctcaaggagttgaaggagatcgacttcagttactaagagacgttggtggagctttcaggcctggcgtattgacagcattggtcggtgttagtggtgcgggtaagactaccttaatggatgtcttggccggtaggaaaacaggaggctatgtagaagggagtattaacatatctggttacccaaagaaccaagcaacatttgctagagtcagtggttactgtgagcaaaatgatatccattctccacatgttaccgtttatgaatcccttatctattcagcatggcttcgtctttccgccgatatagataccaaaacacgagagatgtttgtggaggaagtgatggagttggtggagcttaaacctcttagaaactctatagttggtcttcctggtgtagatggtctttcaacagaacagaggaagaggcttactatagcagttgaattggtagctaatccatcaataatcttcatggacgagccaacatctggtcttgatgcaagagctgctgccattgttatgcgtactgttaggaatactgttgatacaggaagaactgttgtttgtacaattcaccaacctagcatcgacattttcgaatccttcgatgagttgctgttgatgaaacgaggaggacaagttatatatgctggaacactaggacatcactcacaaaaactcgttgaatactttgaggctattgaaggggttccaaagatcaaggacggatacaatcctgcgacgtggatgcttgacgtcactactccttcaatggagtcacaaatgagcgtggactttgctcaaatattcgttaactcctctgttaatcggagaaatcaggaactcatcaaagagctgagtactccaccaccgggatcgaatgatctctacttccgaaccaagtacgcgcaaccattttctactcaaaccaaagcttgcttctggaaaatgtattggtcaaactggagatatcctcagtacaatgccattcggtttctcatgacagtagtcattggtgtcttgtttggtctacttttctggcaaacaggaactaaaatagagaaagagcaagacctgaataatttctttggagccatgtatgctgctgtattgttcctcggtgccaccaacgctgcaacagttcaacccgcagttgctattgagcgaacggttttctaccgcgaaaaagccgctggaatgtactccgccattccctatgcaatttctcaggtagcagtagaaatcatgtacaacactatacaaaccggagtttacacacttatcctttactcgatgatcggatacgactggactgtggtcaaattcttctggttctactactacatgttaacatgcttcgtctacttcaccctatacggtatgatgcttgtcgccttgacaccaaactatcaaatagctggaatctgcttgtccttcttcctcagtttctggaatcttttctccggtttcctcatccctagaccgcaaataccaatatggtggagatggtactattgggcatcacctgtggcttggacattgtatggaatcatcacatctcaagtaggagacagggattcgattgtgcacatcactggtgttggagatatgagtcttaaaacgttgctcaaaaatggattcgggttcgattatgacttcttaccagttgtagccgttgtccacatcgcctggatcttgatctttctctttgcctttgcttatggtatcaagttcctcaacttccaaagaaggtga

SEQ ID NO: 5 - DNA, 6765 bp, Arabidopsis thaliana
  misc_feature 1..6765: note = ORF of protein Acc1
  source 1..6765: mol_type = unassigned DNA
atggctggctcggttaacgggaatcatagtgctgtaggacctggtataaattatgagacggtgtctcaagtggatgagttctgtaaagcacttagagggaaaaggccgatccatagtattttgatagctaacaatggaatggcggctgtgaagtttatacgtagtgtcagaacatgggcttatgaaacatttggtacggaaaaagccatattgttggtggggatggcaacccctgaagacatgcggatcaatgcggagcatatcagaatcgctgatcagtttgttgaggttcccggaggaaccaacaataacaattatgctaacgttcagctgattgtggagatggctgaagtaacacgcgtggatgcagtttggcctggttggggtcatgcatctgaaaaccccgaattacctgatgccctagatgcaaaaggaatcatatttcttggtcctccagcatcttcaatggcagcactgggagataagattggttcttcgttgattgcacaagctgctgatgtacccactctgccatggagtggttcccatgttaaaatacctcctaatagcaacttggtaaccatcccagaggagatctaccggcaagcatgtgtctacacaactgaagaagcgattgctagctgtcaagttgtcggttacccagcaatgatcaaagcatcgtggggtggtggtggtaaaggaatcaggaaggttcataatgatgatgaggttagggctctattcaagcaagttcagggtgaggtcccaggctcaccaatattcataatgaaggttgcgtcacagagtcggcatctagaggtccagctgctctgtgacaagcatggaaatgtttcagctctgcatagccgtgattgtagcgtccagagaagacatcaaaagatcatagaggagggtccaattactgtggctccgccagaaactgtcaagaaacttgaacaagcagctagaaggttggctaagagtgttaactatgttggagctgctactgttgagtatctctacagtatggacactggggagtactacttcttagagcttaaccctcgcttacaggttgagcatcctgtcactgagtggattgccgagataaatcttcctgctgcccaagttgctgtggggatgggaattcctctctggcaaatccctgagataagacggttctatggaatagaacatggtggaggttatgattcttggcgaaaaacatctgttgtagccttcccttttgattttgataaagctcaatctataaggccaaaaggtcattgtgtggctgtacgtgtgacaagtgaggatcctgatgacgggttcaaaccaaccagcggtagagttcaggagttgagttttaagagcaagccaaatgtgtgggcgtacttctctgtcaagtctggtggaggcatccacgagttctcggattcccagtttggacatgtttttgcatttggggaatccagagccctggcgatagcgaatatggttcttgggctaaaagaaattcagatccgtggagaaattaggactaacgttgactacacgatcgaccttttacatgcttctgattaccgtgataacaaaattcacactggttggttggatagtaggattgctatgcgggtcagagctgagaggcctccatggtatctctctgttgtcggcggagctctctataaagcatcagcgaccagtgctgctgtggtttcagattacgttggttatctggagaaggggcaaatccctccaaagcatatatctcttgtacattctcaagtgtctctgaatattgaaggaagtaaatatacgattgatgtagtccggggtggatcaggaacctacaggctaagaatgaacaagtcagaagtggtagcagaaatacacactctacgtgatggaggtctgttgatgcagttggatggcaaaagccatgtgatatatgcagaggaagaagctgcaggaactcgtcttctcattgatggaagaacttgtttgctacagaatgaccacgatccatcaaagttaatggctgagacaccgtgcaagttgatgaggtatttgatttccgacaacagcaatattgacgctgatacgccttatgccgaagttgaggtcatgaagatgtgcatgccacttctttcacctgcttcaggagttatccattttaaaatgtctgaaggacaagccatgcaggctggtgaacttatagccaatcttgatcttgatgatccttctgctgtaagaaaggccgaacccttccatggaagtttcccaagattagggcttccaactgcaatatccggtagagttcatcagagatgtgccgcaacattaaatgctgcacgcatgattcttgctggctatgagcataaagtagatgaggttgttcaagacttacttaattgccttgatagccctgaactcccatttcttcagtggcaagagtgctttgcagttctggcgacacgactacctaaaaatctcaggaacatgctagaatcaaagtatagggaatttgagagtatttccagaaactctttgaccaccgatttccctgccaaacttttaaaaggcattcttgaggcacatttatcttcttgtgatgagaaagagagaggtgcccttgaaaggctcattgaaccattgatgagccttgcaaaatcttatgaaggtggtagagaaagtcatgcccgtgttattgttcattctctctttgaagaatatctatcagtagaagaattattcaatgataacatgctggctgatgttatagaacgcatgcgtcagctatacaagaaagatctgttgaaaattgtggatatagtgctctcacaccagggcataaaaaacaaaaacaaactcgttctccggctcatggagcagcttgtttaccctaatcctgctgcttacagagataaacttattcgattctcaacacttaaccatactaactactctgagttggcgctcaaggcgagtcaattacttgaacagaccaaactaagtgagcttcgttcaaacattgctagaagcctttcagagttagaaatgtttacagaggacggagaaaatatggatactcccaagaggaaaagtgccattaatgaaagaatagaagatcttgtaagcgcatctttagctgttgaagacgctctcgtgggactatttgaccatagcgatcacacacttcaaagacgggttgttgagacttatattcgcagattataccagccctacgtcgttaaagatagcgtgaggatgcagtggcaccgttctggtcttcttgcttcctgggagttcctagaggagcatatggaaagaaaaaacattggcttagacgatcccgacacatctgaaaaaggattggttgagaagcgtagtaagagaaaatggggggctatggttataatcaaatctttgcagtttcttccaagtataataagtgcagcattgagagaaacaaagcacaacgactatgaaactgccggagctcctttatctggcaatatgatgcacattgctattgtgggcatcaacaaccagatgagtctgcttcaggacagtggggatgaagaccaagctcaggaaagagtaaacaagttggccaaaattcttaaagaggaagaagtgagttcaagcctctgttctgccggtgttggtgtaatcagctgtataattcagcgagatgaaggacgaacacccatgagacattctttccattggtcgttggagaaacagtattatgtagaagagccgttgctgcgtcatcttgaacctcctctgtccatttaccttgagttggataagctgaaaggatactcaaatatacaatatacgccttctcgagatcgtcaatggcatctgtatactgttacagacaagccagtgccaatcaagaggatgttcctgagatctcttgttcgacaggctacaatgaacgatggatttatattgcagcaagggcaggataagcagcttagccaaacactgatctccatggcgtttacgtcgaaatgtgttctgaggtctttgatggatgccatggaggaactggaactgaatgcccataatgctgcaatgaaaccagatcacgcacatatgtttctttgcatattgcgtgagcagcagatagatgatcttgtgcctttccccaggagagttgaagtgaatgcggaggatgaagaaactacagttgaaatgatcttagaagaagcagcacgagagatacatagatctgttggagtgagaatgcataggttgggcgtgtgcgagtgggaagtgcggctgtggttggtgtcctctggactggcatgtggtgcttggagggttgtggttgcaaacgtgacaggccgtacatgcactgtccacatataccgagaagttgaaactcctggaagaaacagtttaatctaccactcaataaccaagaagggacctttgcatgaaacaccaatcagtgatcaatataagcccctgggatatctcgacaggcaacgtttagcagcaaggaggagtaacactacttattgctatgacttcccgttggcatttgggacagccttggaactgttgtgggcatcacaacacccaggagttaagaaaccatataaggatactctgatcaatgttaaagagcttgtattctcaaaaccagaaggttcttcgggtacatctctagatctggttgaaagaccacccggtctcaacgactttggaatggttgcctggtgcctagatatgtcgaccccagagtttcctatggggcggaaacttctcgtgattgcgaatgatgtcaccttcaaagctggttcttttggtcctagagaggacgcgtttttccttgctgttactgaactcgcttgtgccaagaagcttcccttgatttacttggcagcaaattctggtgcccgacttggggttgctgaagaagtcaaagcctgcttcaaagttggatggtcggatgaaatttcccctgagaatggttttcagtatatatacctaagccctgaagaccacgaaaggattggatcatctgtcattgcccatgaagtaaagctctctagtggggaaactaggtgggtgattgatacgatcgttggcaaagaagatggtattggtgtagagaacttaacaggaagtggggccatagcgggtgcttactcaaaggcatacaatgaaacttttactttaacctttgttagtggaagaacggttggaattggtgcttatcttgcccgcctaggtatgcggtgcatacagagacttgatcagccgatcatcttgactggcttctctacactcaacaagttacttgggcgtgaggtctatagctctcacatgcaactgggtggcccgaaaatcatgggcacaaatggtgttgttcatcttacagtctcagatgatcttgaaggcgtatcagcaattctcaactggctcagctacattcctgcttacgtgggtggtcctcttcctgttcttgcccctttagatccaccggagagaattgtggagtatgtcccagagaactcttgcgacccacgagcggctatagctggggtcaaagacaataccggtaaatggcttggaggtatctttgataaaaatagtttcattgagactcttgaaggctgggcaaggacggtagtgactggtagagccaagctcgggggaatacccgttggagttgttgcagttgagacacagactgtcatgcagatcatcccagccgatcctggacagcttgactctcatgaaagagtggttccgcaagcagggcaagtctggtttcctgattcagcggccaagactgctcaagcgcttatggatttcaaccgggaagagcttccattgtttatcctagcgaactggagagggttttcaggtgggcagagagatcttttcgaaggaatacttcaggcaggttcaactatagtagaaaatctgagaacctatcgtcagccagtgtttgtgtacatcccaatgatgggagagctgcgcggtggagcgtgggttgttgttgacagccagataaattcggattatgttgaaatgtatgctgatgaaacagctcgtggaaatgtgcttgagccagaagggacaatagagataaaatttagaacaaaagagctattagagtgcatgggaaggttggaccagaagctaatcagtctgaaagcaaaactgcaagatgccaagcaaagcgaggcctatgcaaacatcgagcttctccagcaacagattaaagcccgagagaaacagcttttaccagtttatatccaaatcgccaccaaatttgcagaacttcatgacacttccatgagaatggctgcaaagggagtgatcaaaagtgttgtggaatggagcggctcgcggtccttcttctacaaaaagctcaataggagaatcgctgagagctctcttgtgaaaaacgtaagagaagcatctggagacaacttagcatataaatcttcaatgcgtctgattcaggattggttctgcaactctgatattgcaaaggggaaagaagaagcttggacagacgaccaagtgttctttacatggaaggacaatgttagtaactacgagttgaagctgagcgagttgagagcgcagaaactactgaaccaacttgcagagattgggaattcctcagatttgcaagctctgccacaaggacttgctaatcttctaaacaaggtggagccgtcgaaaagagaagagctggtggctgctattcgaaaggtcttgggttga

SEQ ID NO: 6 - DNA, 1134 bp, Arabidopsis thaliana
  misc_feature 1..1134: note = ORF of protein Agb1
  source 1..1134: mol_type = unassigned DNA
atgtctgtctccgagctcaaagaacgccacgccgtcgctacggagaccgttaataacctccgtgaccagcttagacagagacgcctccagctcctcgataccgatgtggcgaggtattcagcggcgcaaggacgtactcgggtgagcttcggagcaacggatctggtttgttgtcgtactcttcagggacacaccggaaaggtttattcattagattggacaccggagaggaaccggattgtcagtgcatctcaagatgggagattaatcgtgtggaatgctctaacgagtcagaaaactcatgctattaaactcccttgtgcatgggttatgacatgtgctttctctccaaatggtcagtcggttgcgtgtggtggattagacagtgtatgttctatctttagccttagctcaacggcggacaaggatggaactgtaccggtttcaagaatgctcactggtcacaggggatatgtttcgtgctgtcagtatgtcccaaatgaggatgcccaccttatcaccagttcaggtgatcaaacttgtatcttatgggatgtaactactggtctcaaaacttctgtttttggcggtgaatttcagtctggacatactgctgatgtactaagcgtctcaatcagtggatcaaacccaaactggtttatatctggttcatgcgattccacagcacggttgtgggacactcgtgctgcaagccgagcagtgcgtacctttcatggtcacgagggagatgttaatacggtcaagttctttccggatgggtatagatttgggactggatcagacgatggaacatgcaggctgtatgacataaggactggtcaccaactccaggtctatcagccacatggtgatggtgagaacggacctgtcacctccattgcattctctgtgtcagggagacttcttttcgctggctatgcgagcaacaacacttgctacgtttgggataccctcttgggagaggttgtattggatttgggattacagcaggattcacacaggaatagaataagctgtttggggttgtcagcagatggaagtgccttgtgtacaggaagttgggattcaaatctaaagatatgggcgtttggaggacacaggagagtgatttga

SEQ ID NO: 7 - DNA, 2013 bp, Arabidopsis thaliana
  misc_feature 1..2013: note = ORF of protein Als
  source 1..2013: mol_type = unassigned DNA
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatactga

SEQ ID NO: 8 - DNA, 1134 bp, Arabidopsis thaliana
  misc_feature 1..1134: note = ORF of protein Anac076
  source 1..1134: mol_type = unassigned DNA
atggaatcggtggatcaatcatgtagtgttcctccgggattcagattccatccaacagatgaagagctcgttggttactatttaaggaaaaaagttgcatcgcaaaagatcgatcttgatgtcataagagatattgatctctacagaatcgaaccatgggatttacaagagagctgccgaatcggatatgaggaaagaaatgaatggtatttcttcagccacaaagataagaaatatccaacaggaacaagaacaaacagagcgaccatggctgggttttggaaagccacgggccgagacaaggctgtttacgacaagtcaaaactgattggtatgagaaaaacacttgtgttctacaaaggaagagcccctaatggccaaaaaaccgattggatcatgcatgaataccggctagagtcagatgagaatgcacctcctcaggaagaagggtgggtggtttgtagagcgttcaagaaaaagccaatgaccgggcaagccaagaacacagaaacttggagctcaagttacttttacgacgaattaccgagtggagtacgctcagttacggagcctcttaactacgtatctaagcagaaacaaaacgtttttgcacaagatttaatgttcaagcaagaactagaaggatcagatatcggtttaaacttcatccactgcgatcaattcattcaacttccgcagcttgaaagcccttcactccctcttaccaaaagaccagtgagcttgacgtcaattacatcattggagaagaataaaaatatctacaaaagacatttaatagaagaggatgtgagcttcaatgcgctaataagtagtggaaataaagataagaagaagaagaaaacatcagtgatgacgacggattggagagcactcgataaatttgttgcttctcaacttatgagccaagaagatggagtttcgggttttggaggtcatcatgaagaagataacaataaaatcggtcattacaataacgaagagagcaataacaagggatcagtagagactgcttcttccacgttattgagtgatagagaagaagagaacagattcatcagtggattattgtgttccaacttggactatgacttatatagggatttacatgtttga

SEQ ID NO: 9 - DNA, 2601 bp, Arabidopsis thaliana
  misc_feature 1..2601: note = ORF of protein Apg9
  source 1..2601: mol_type = unassigned DNA
atgatgagcagtgggcataagggtccaaatgtacgtaacttttttaaatggcaacgtggtgaatcatcatcgtctttgactactggattgcttcataatgagtcacacgagattgagctatctaactacggaggaataccaagtcctggtagtgagagcccatcgggacttcttaatggagagagtttaaatgttcaaccaattgctgatttggatctttttgtcgaaaggctctacagctactacagagacaaaggactttggtgtattatagtaaagtgggctgttgagcttctaagtctgggcttcatcatatgcttctctgggtttttcttgctgtatgttgattggaatggtcttcagaatgcaaaatgtggaatggatgcagttgaatcaggaactaaaccatgtgatcttgtcaaagaagctatccatccgcatcctttatcccctttcacgctcacaactgctataattgttggatatttggccctcttctctgtgtactggctcttctgcttcttaaggttttttgcccagttgaaagatactttggactttcggcacttttattacaacaatctccatgtcacagacaatgaaatccttactatgccatgggcaacagtccttgagaaggttgttcagttacaaagctcacaatgcctttgtgtggttaaggatctttctgctcatgatatggttatgcgccttatgcggaaagaaaactacttgattggaatgctcaacaaaggtttgctttctttcccaatttcccattggatccctggtgctggtccagcagtcaaatccgcaccagatgggacacagtatcaccttgtattgacaaagacccttgagtggacccttaactggtgcatattgcagagcatgtttgactgcaattttcgtgttagaagggactttgtttcaaatcctacaacactgaagaaacgactatttgtggttgggcttgctatgcttctgctgtcaccgtttcttgtcatctttatgttggtgtatttattcctaaggcatgctgaacagttctacaatcatccaagtactgcatcttctcgaagatggtccaatttgtcaaagtggctcttcagggaattcaatgaggttgaccatttattcaagcatcggattaatagcagtgtagtacatgcatccgaatatctaaagcaatttccctcgcctatcatctccattattgcaaagtttgtctcgtttgtttctggcggctttgctgctgtcttaatcatcattgcgtttcttgaagaatctctacttgagggacatatattcggccgcaatctgttctggtatgccgctgtttttggaacaattacagctattagtagagctgccatatcggatgaacttttggtcctcgacccagtaggaactatgtctttggtggtccaaaacactcattatatgcctaagagatggcgtggaaaggagaacaaggatgatgtccgcttggagttagagactctgtttcagtatactggaatgatgctattggaggagatagcttcaatcttcatcacaccatttttgctgatgtttgtcgtgccaaagcgggtggatgacatcttgcaattcattaaggatttcacagttgatattgaaggtgtaggccatgtttgcagctttagtgctttctactttgagaaccatggaaatatcaaatatggttcaccacataatgcaactcgtcgtgagcagagaagctcacaagggaaaatggagaaatcattcttgagtttccagagtagttatccttcttgggagtcagattctcttgggaagcagtttctgtcaaatctcagaactttccgggaccgaaagcttcatgaaataaacacaagacactcatctcctagtagggcgtggcgagaaagcactaatacccctgctttatacagagacattccaagaaatccgcttgcgagcggcaatcacactgattcaatgtggctgattgatccagatcagaggaatcatccttatcttcttgactggtattacacttctcaggctcacaacagaactgatcacccaatagagagagcaaatgaaatcttaaccgcaaatcagaacgctacagattgttggcctcctgatttgggaatccgtggtgaagatagcagagatctgctcaacatggaggcaagcacatcaggtcagtttttcagagagagcattctgcgccatgaccaacccgagggagaggatagttatgggagtcagcatcctcttgatggtaggaaccagtggtggggaagaggcaaccactctcagatcagcacagctcatcccgcaaccaccaatagcttcatcgagccacctgattttataaaccgctacacagcaggaaacttattagacaactcttggagcagaagaagcatagaagaagaagatgaagaagaagaagaattggattgggaagagaatgcaagaagaaatttgtcgagaactacattcatggacgataacgatatcgaagctgggattgatctccatttcgacgatgtatatagttcaagacctcaagaaacttcaacatcaagcaccacattacggtga

SEQ ID NO: 10 - DNA, 618 bp, Arabidopsis thaliana
  misc_feature 1..618: note = ORF of protein Arlb1
  source 1..618: mol_type = unassigned DNA
atgttttctcttatgtctggactatggagttacatgtttagcaagactgagtttaatgtgcttattcttggtatcgacaaagctgggaagacgacatttttggagaaactgaagacgatatactcaatctcggaaggtcttcctcatgatcgaattgttcctactgtggggcttaatattggtcgcattgaagtctccaatgctaaaattgtgttctgggatctgggaggtcagcctggtttgcggtctatttgggagaaatactatgaagaagcacacgcgttgatatatttgattgatgcagcttgcccaacccgctttgaagattctaaatctgctttagaaaaagccctgcggcatgaggatttgcaaggagcccctcttttaatattggcaaacaagcaagacctcacaaatgctgtatcggctgaggaactggaccgataccttgatttaaagaaattggatgaaagagtgtacatgtttgaagcagtttctggatatgatgggcgaggaatcaaagaaagtatagaatggctggttggagtaatggagaaaagcaagagaaccgaatcattaagagcccgtgcagggtacactcctgtgccaaattcatag

SEQ ID NO: 11 - DNA, 2073 bp, Arabidopsis thaliana
  misc_feature 1..2073: note = ORF of protein Arr1
  source 1..2073: mol_type = unassigned DNA
atgatgaatccgagtcacggaagaggactcggatcggctggtgggtccagctccggtagaaatcaaggaggtggtggtgagaccgtcgtcgagatgtttccttctggtcttcgagttcttgtcgttgacgatgacccaacttgtctcatgatcttagagaggatgcttaggacttgtctttacgaagtaacgaaatgcaacagagcagagatggcattgtctctgctccggaagaacaaacatggattcgatatagtaatcagtgatgttcatatgcctgacatggacggtttcaagcttcttgagcatgttggtctagagatggacttacctgttatcatgatgtctgcggatgattcaaagagtgtggttctaaagggagtaacgcacggtgcggttgattaccttatcaagcctgtacgtatggaggcacttaagaacatatggcagcatgtagttaggaagaggagaagtgaatggagtgtaccggaacattctgggagcattgaggagactggcgagagacagcagcagcaacatagaggaggtggtggtggtgcagctgtttctggtggagaggatgcggtggatgataactcatcctcggttaacgaaggtaacaattggaggagcagttcacggaagaggaaagacgaggaaggagaagagcaaggagatgataaggacgaagatgcgtcgaatttgaagaaaccgcgtgtcgtctggtctgttgaattgcatcagcagtttgttgctgctgttaatcagctcggcgttgagaaggcggttcctaaaaagatcttagagctgatgaatgttcctggtctaacccgagaaaacgtagcaagtcacctccagaaataccggatatatctaagacggcttggaggggtatcgcagcaccaaggcaatcttaacaactcgtttatgacgggtcaggatgcgagcttcggacctctttcgacattgaatgggtttgatcttcaagcactagccgtcacaggtcagttacctgcacagagtcttgcacagcttcaagccgctggtttaggccggcctgcgatggtctctaagtcaggtttgccggtttcctccattgtggatgagagaagcatcttcagctttgacaacacgaaaacaagatttggagaagggcttgggcatcacgggcaacaaccccaacagcaaccacagatgaacttacttcacggtgtccccacgggtttacaacagcagcttcctatgggtaatcgaatgagtattcaacaacagattgctgctgttcgagctggaaatagtgttcaaaacaacggaatgctgatgcctctagcgggtcagcagtctttgcctcggggaccaccgcctatgctaacctcttcgcaatcatccatcaggcagccgatgttatcaaaccgcatttccgagagaagtggtttctctggaaggaacaatatccccgagagcagcagagtgttaccgacaagttacactaatctcacaacacaacactcatcaagctcgatgccttataacaacttccaaccagaacttcccgtgaacagtttcccgctggcaagtgcaccagggatatcagtaccggttcggaaagccacttcttaccaggaagaggttaacagctccgaagcgggtttcactacgccgagctacgacatgttcaccaccagacagaatgattgggatctgaggaatattggaatagcctttgactcacatcaggactcagaatccgctgcgttttccgcttcagaagcctactcttcttcgtccatgtcaagacacaacacgacagttgcagccaccgagcatggccgaaaccaccagcagccaccatcgggaatggtacagcaccatcaggtttatgcagacggaaacggtggttcagtgagggtgaaatcagagagagtggctacggatacagcaacaatggcgtttcacgagcagtatagtaatcaagaagatcttatgagcgcacttcttaagcaggaagggattgcaccggttgatggcgaattcgactttgacgcatactccatcgataacattccggtttga

SEQ ID NO: 12 - DNA, 555 bp, Arabidopsis thaliana
  misc_feature 1..555: note = ORF of protein Arr5
  source 1..555: mol_type = unassigned DNA
atggctgaggttttgcgtcccgagatgttagatatctctaacgacacttcttcattagcatcaccgaaacttcttcatgttcttgctgttgatgatagtatggttgatcggaagttcatcgagcggttactcagagtctcatcttgtaaagttactgttgtcgatagtgcgacaagagctttacaatatcttggattagatggagagaataatagttcggttggatttgaggatctgaagattaatttgataatgacggattactctatgcctgggatgactggatatgaactactgaagaagatcaaagaatcatcagctttcagagaaataccagttgtgattatgtcttcagagaacatcttgcctcgtatcgatagatgtcttgaagaaggagctgaagatttcttattgaagcctgtgaaattagctgatgtgaagagattaagagattctttaatgaaagctgaggaaagagcttttaagaatattatgcacaagagagagcttgaagctaatgatatctactcgcagctaaaacgcgcaaagatctga

SEQ ID NO: 13 - DNA, 561 bp, Arabidopsis thaliana
  misc_feature 1..561: note = ORF of protein Arr6
  source 1..561: mol_type = unassigned DNA
atggctgaagttatgctaccgaggaagatggagattctcaaccattcttcaaagtttggatcaccggatcctcttcatgttcttgccgtcgacgacagtcacgttgatcgtaaattcatcgagcgtttgctcagagtatcttcttgcaaagttactgttgttgatagtgcaacaagagctctccaataccttggtttggatgttgaggaaaaatcagtcggttttgaggatttgaaggttaatttaattatgactgattactcaatgcccggtatgactggatatgaactcttgaagaagatcaaagaatcctcagcttttagagaagtaccggtggtaattatgtcctccgagaacattttgcctcgtattgatagatgtcttgaagaaggggctgaagatttcttactgaagccggtaaaactctcggatgtaaaaagattaagagattctctaatgaaggttgaagatttgtctttcacaaagagtattcagaagagagagctagaaacagagaatgtctaccctgttcactcgcagctcaaacgcgcaaagatctga

SEQ ID NO: 14 - DNA, 1380 bp, Arabidopsis thaliana
  misc_feature 1..1380: note = ORF of protein At59
  source 1..1380: mol_type = unassigned DNA
atggcagcagctttcttgaatcttggcggctatgttttcgttttcttctcttcgttcttagctatagtggccccacaggttcgaggcaatgttgctgtctttgatagctactggacgcaacgacaaagtgatgcattgaaacaaaccatcggctcttacgatcctcatccacttaacgtcaccaaccattttaattaccatgtcaatatagcagtggatgcatcggaatcaaggaacgatacaaggagagagcttactcaggtgagaagtggtcgtaaaacgcacaaaagcagcggaaaatgcctagcttataacccgatagacaactgctggcgatgcgatcgtaactgggctaataaccggaagaaactagccgactgtgtgctcggattcggccggagaaccaccggaggaaaagacggaccgatatacgtcgtcaaagacgcgtctgacaacgatcttatcaaccctaaaccaggaacgttaaggcatgcagtgactcgagacggaccgctttggatcatattcgcaagaagcatgatcataaaactgcaacaagaactgatgataacgagcgacaaaacaatcgatggaagaggagctagggtttacatcatggaaggtgctggattaacgttgcaatttgtgaataatgtgatcattcacaatatttatgtcaagcacattgttcctgggaacggtgggttgatcagagactctgaggctcacatagggcttaggacaaaaagtgatggtgatggaatcagtttgtttggagcaaccaatatttggatcgaccatgtctcaatgacaagatgtgcagatggtatgatcgatgctattgacggctccactgctgtaactatttccaacagtcatttcaccgaccaccaagaggtgatgttgtttggggcgagggacgaacacgtgatcgacaagaaaatgcagataacggttgcgtttaaccacttcggtaagcgattggaacagagaatgcctcgttgcagatacggaacgatacatgtggtgaacaatgactacacacattgggaaatgtatgcaattggtgggaacatgaaccctacaattattagtcaaggcaatcgtttcattgctcctcctaatgaagaagccaaacagattacaaaaagagagtacacaccatacggtgaatggaaatcttggaattggcaatcagaaggagattactttctcaacggagcttattttgtgcaatccggaaaagcaaatgcgtggagctctaaaccgaaaaccccgctcccaaacaagtttacaattcgacctaaaccgggaacgatggtccggaaacttaccatggatgcaggagtacttggctgcaaacttggtgaagcttgctga

SEQ ID NO: 15 - DNA, 1848 bp, Arabidopsis thaliana
  misc_feature 1..1848: note = (ORF of protein Bak1
  source 1..1848: mol_type = unassigned DNA
atggaacgaagattaatgatcccttgcttcttttggttgattctcgttttggatttggttctcagagtctcgggcaacgccgaaggtgatgctctaagtgcactgaaaaacagtttagccgaccctaataaggtgcttcaaagttgggatgctactcttgttactccatgtacatggtttcatgttacttgcaatagcgacaatagtgttacacgtgttgaccttgggaatgcaaatctatctggacagctcgtaatgcaacttggtcagcttccaaacttgcagtacttggagctttatagcaataacattactgggacaatcccagaacagcttggaaatctgacggaattggtgagcttggatctttacttgaacaatttaagcgggcctattccatcaactctcggccgacttaagaaactccgtttcttgcgtcttaataacaatagcttatctggagaaattccaaggtctttgactgctgtcctgacgctacaagttctggatctctcaaacaatcctctcaccggagatattcctgttaatggttccttttcacttttcactccaatcagttttgccaacaccaagttgactccccttcctgcatctccaccgcctcctatctctcctacaccgccatcacctgcagggagtaatagaattactggagcgattgcgggaggagttgctgcaggtgctgcacttctatttgctgttccggccattgcactagcttggtggcgaaggaaaaagccgcaggaccacttctttgatgtaccagctgaagaggacccagaagttcatttaggacaactgaagaggttttcattgcgtgaactacaagttgcttcggataattttagcaacaagaacatattgggtagaggtggttttggtaaagtttataaaggacggttagctgatggtactttagtggccgttaaaaggctaaaagaggagcgcacccaaggtggcgaactgcagttccagacagaggttgagatgattagtatggcggttcacagaaacttgcttcggcttcgtggattttgcatgactccaaccgaaagattgcttgtttatccctacatggctaatggaagtgttgcctcctgtttaagagaacgtcccgagtcccagccaccacttgattggccaaagagacagcgtattgcgttgggatctgcaagagggcttgcgtatttacatgatcattgcgacccaaagattattcatcgagatgtgaaagctgcaaatattttgttggatgaagagtttgaagccgtggttggggattttggacttgcaaaactcatggactacaaagacacacatgtgacaaccgcagtgcgtgggacaattggtcatatagcccctgagtacctttccactggaaaatcatcagagaaaaccgatgtctttgggtatggagtcatgcttcttgagcttatcactggacaaagggcttttgatcttgctcgcctcgcgaatgatgatgatgtcatgttactagactgggtgaaagggttgttaaaagagaagaaattggaagcactagtagatgttgatcttcagggtaattacaaagacgaagaagtggagcagctaatccaagtggctttactctgcactcagagttcaccaatggaaagacccaaaatgtctgaagttgtaagaatgcttgaaggagatggtttagctgagagatgggaagagtggcaaaaggaggaaatgttcagacaagatttcaactacccaacccaccatccagccgtgtctggctggatcattggcgattccacttcccagatcgaaaacgaatacccctcgggtccaagataa

SEQ ID NO: 16 - DNA, 843 bp, Arabidopsis thaliana
  source 1..843: mol_type = unassigned DNA
  misc_feature 1..843: note = ORF of protein Bccp1
atggcgtcttcgtcgttctcagtcacatctccagctgctgctgcttccgtctatgcagtcactcaaacctcctcgcacttcccaatccaaaaccgctctcgcagagtttctttccgtctctctgctaagcccaagcttcgctttctctccaagcctagtcgcagtagctaccctgtggtgaaagcacaatctaacaaggttagtactggtgcatcatcaaatgctgccaaagttgatgggccatcatcagctgaaggaaaggagaaaaactcattgaaggagtcgtctgcttcttctcctgaattagctacagaagagtctatttctgagttccttacccaagtaacaactcttgtcaagcttgtggattcaagagacattgttgagttgcagttgaaacaactcgactgtgaactagtcattcgaaaaaaagaagccttacctcaacctcaagctcctgcatcttatgttatgatgcagcaaccaaatcaaccatcttatgcccagcaaatggctcctcctgctgcacctgctgctgccgcaccagccccttctacgccagcctctctgcctccaccatccccacctactccagccaaatcgtcacttcctactgttaaaagccccatggctggcacattctaccgtagtcctgcacctggtgaaccaccctttattaaggttggagacaaagtgcagaaggggcaagttctatgcattgttgaagccatgaagttaatgaatgaaatagagtctgaccataccggaaccgtagtcgatattgtcgcagaagacggcaagcctgtcagcctcgacactcctctgtttgtggttcaaccgtag

SEQ ID NO: 17 - DNA, 768 bp, Arabidopsis thaliana
  misc_feature 1..768: note = ORF of protein Bccp2
  source 1..768: mol_type = unassigned DNA
atggcgtcattgtcggtaccttgcgtcaagatctgtgctctgaacaggcgggtcggatctctccctggaatctctactcaaagatggcagccgcaacccaatgggatctcctttccctccgatgtttctcagaatcattctgcattctggaggttgcgtgcaacaactaatgaggttgtttctaactccactccaatgactaatggtgggtatatgaacggaaaagcgaaaaccaatgttcctgaacccgccgagctctctgaatttatggctaaagtctctggtcttcttaagcttgtggattcaaaagacatagtggaacttgaactaaagcagctcgactgtgagatcgttattcgaaagaaggaagctttacagcaagctgtaccaccagctccagtttatcactcaatgcctcctgtaatggcagacttttcaatgcctccagctcaaccagtggctcttcctccttctcctactcctacctcaacgcctgcaacagcaaaaccaacatccgccccatcctcgtctcatcctccactcaagagtcccatggctggtactttctatagatctcctggacccggtgaacccccttttgtaaaggttggagataaggtacagaagggtcaaattgtttgtattatcgaagctatgaaactgatgaacgagattgaggctgagaagtcaggaaccatcatggaactactggctgaagatggaaaaccagtcagcgttgacacgcctctttttgtcatcgcaccttga

SEQ ID NO: 18 - DNA, 3591 bp, Arabidopsis thaliana
  misc_feature 1..3591: note = ORF of protein Bri1
  source 1..3591: mol_type = unassigned DNA
atgaagactttttcaagcttctttctctctgtaacaactctcttcttcttctccttcttttctctttcatttcaagcttcaccatctcagtctttatacagagaaatccatcagcttataagcttcaaagacgttcttcctgacaagaatcttctcccagactggtcttccaacaaaaacccgtgtactttcgatggcgttacttgcagagacgacaaagttacttcgattgatctcagctccaagcctctcaacgtcggattcagtgccgtgtcctcgtctctcctgtctctcaccggattagagtctctgtttctctcaaactcacacatcaatggctccgtttctggcttcaagtgctctgcttctttaaccagcttggatctatctagaaactctctttcgggtcctgtaacgactctaacaagccttggttcttgctccggtctgaagtttcttaacgtctcttccaatacacttgattttcccgggaaagtttcaggtgggttgaagctaaacagcttggaagttctggatctttctgcgaattcaatctccggtgctaacgtcgttggttgggttctctccgatgggtgtggagagttgaaacatttagcgattagcggaaacaaaatcagtggagacgtcgatgtttctcgctgcgtgaatctcgagtttctcgatgtttcctccaacaatttctccactgggattcctttcctcggagattgctctgctctgcaacatcttgacatctccgggaacaaattatccggcgatttctcccgtgctatctctacttgcacagagctcaagttgttgaacatctctagtaaccaattcgtcggaccaatccctccgctaccgcttaaaagtctccaatacctctctctggccgagaacaaattcaccggcgagatccctgactttctctccggcgcgtgtgatacactcactggtctcgatctctctggaaatcatttctacggtgcggttcctccattcttcggttcatgttctcttctcgaatcactcgcgttgtcgagtaacaacttctctggcgagttaccgatggatacgttgttgaagatgagaggactcaaagtacttgatctgtctttcaacgagttttccggcgaattaccggaatctctgacgaatctatccgcttcgttgctaacgttagatctcagctccaacaatttctccggtccgattctcccaaatctctgccagaaccctaaaaacactctgcaggagctttaccttcagaacaatggcttcaccgggaagattccaccgactttaagcaactgttctgagctggtttcgcttcacttgagcttcaattacctctccgggacaatcccttcgagcttaggctctctatcgaagcttcgagatctgaaactatggctgaatatgttagaaggagagatccctcaggagctcatgtatgtcaagaccttagagactctgatcctcgacttcaacgatttaaccggtgaaatcccttccggtttaagtaactgtaccaatcttaactggatttctctgtcgaataaccggttaaccggtgagattccgaaatggattggccggttagagaatctcgctatcctcaagctaagcaacaattcattctccgggaacattccggctgagctcggcgactgcagaagcttaatctggcttgatctcaacaccaatctcttcaatggaacgattccggcggcgatgtttaaacaatccgggaaaatcgctgccaatttcatcgccggtaagaggtacgtttatatcaaaaacgatgggatgaagaaagagtgtcatggagctggtaatttacttgagtttcaaggaatcagatccgaacaattaaaccggctttcaacgaggaacccttgtaatatcactagcagagtctatggaggtcacacttcgccgacgtttgataacaatggttcgatgatgtttctggacatgtcttacaacatgttgtctggatacataccgaaggagattggttcgatgccttatctgtttattctcaatttgggtcataacgatatctctggttcgattcctgatgaggtaggtgatctaagaggtttaaacattcttgatctttcaagcaataagctcgatgggaggattcctcaggctatgtcagctcttactatgcttacggaaatcgatttgtcgaataataatttgtctggtccgattcctgagatgggtcagtttgagacttttccaccggctaagttcttgaacaatcctggtctctgtggttatcctcttccgcggtgtgatccttcaaatgcagacggttatgctcatcatcagagatctcatggaaggagaccagcgtcccttgctggtagtgtggcgatgggattgttgttctcttttgtgtgtatatttgggctgatccttgttggtagagagatgaggaagagacggagaaagaaagaggcggagttggagatgtatgcggaaggacatggaaactctggcgatagaactgctaacaacaccaattggaagctgactggtgtgaaagaagccttgagtatcaatcttgctgctttcgagaagccattgcggaagctcacgtttgcggatcttcttcaggctaccaatggtttccataatgatagtctgattggttctggtgggtttggagatgtttacaaagcgattttgaaagatggaagcgcggtggctatcaagaaactgattcatgttagcggtcaaggtgatagagagttcatggcggagatggaaaccattgggaagatcaaacatcgaaatcttgtgcctcttcttggttattgcaaagttggagacgagcggcttcttgtgtatgagtttatgaagtatggaagtttagaagatgttttgcacgaccccaagaaagctggggtgaaactaaactggtccacacggcggaagattgcgataggatcagctagagggcttgctttccttcaccacaactgcagtccgcatatcatccacagagacatgaaatccagtaatgtgttgcttgatgagaatttggaagctcgggtttcagattttggcatggcgaggctgatgagtgcgatggatacgcatttaagcgtcagtacattagctggtacaccgggttacgttcctccagagtattaccaaagtttcaggtgttcaacaaaaggagacgtttatagttacggtgtggtcttactcgagctactcacgggtaaacggccaacggattcaccggattttggagataacaaccttgttggatgggtgaaacagcacgcaaaactgcggattagcgatgtgtttgaccccgagcttatgaaggaagatccagcattagagatcgaacttttacaacatttaaaagttgcggttgcgtgtttggatgatcgggcttggagacgaccgacaatggtacaagtcatggccatgtttaaggagatacaagccgggtcagggatagattcacagtcaacgatcagatcaatagaggatggagggttcagtacaatagagatggttgatatgagtataaaagaagttcctgaaggaaaattatga

SEQ ID NO: 19 - DNA, 753 bp, Arabidopsis thaliana
  misc_feature 1..753: note = ORF of protein Bzo2h3
  source 1..753: mol_type = unassigned DNA
atggaaaaagttttctccgacgaagaaatctccggtaaccatcactggtcggttaatggaatgacgtcgttgaatcgcagtgcttccgaatgggcattcaatcgtttcatacaagaatcctccgccgctgcagacgacggagaatctacgacggcgtgtggtgtttccgtctcctctcctcctaatgttcctgtagattcagaggaatacagagcatttctcaagagtaaacttaatcttgcttgtgctgctgtcgccatgaaaaggggaactttcatcaaacctcaggatacttctggtagatctgacaatggtggagccaatgaatcagaacaagcctctcttgcttcttccaaagctacaccaatgatgagcagtgctataacaagtggatctgagctctctggtgatgaagaagaagctgatggtgaaactaatatgaaccctactaatgttaaacgcgttaaaaggatgctctctaatagagaatcagctagacggtccagaagaagaaagcaagcacacttgagtgagctagagacacaagtttcacagcttcgtgtagagaattcaaaactcatgaagggtctcactgatgtaactcaaacattcaatgatgcatctgtagaaaacagagttttaaaagccaatattgagacactacgagcaaaggtaaaatactttcttttatctctctgtctaccaaaactcgctattgaagcatgtttgtttttggcaggtgaaaatggctga

SEQ ID NO: 20 - DNA, 3255 bp, Arabidopsis thaliana
  misc_feature 1..3255: note = ORF of protein Cesa6
  source 1..3255: mol_type = unassigned DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 21 - DNA, 1356 bp, Arabidopsis thaliana
  misc_feature 1..1356: note = ORF of protein Cipk3
  source 1..1356: mol_type = unassigned DNA
atgttgatccccaacaaaaaattaagagaaatgaatcggagacagcaagtgaaacgtagagtaggtaaatatgaagttggaagaacaattggagaaggaacgtttgctaaagttaagtttgctagaaactctgaaactggagaacctgttgctctcaagattcttgataaagagaaagttctcaagcataaaatggctgaacagattagaagagagatagctactatgaagttgataaaacatccaaatgttgttcaattatatgaggtgatggcaagtaagacgaaaatatttatcatcttggagtatgttacaggaggagaactctttgataagattgtaaatgatgggcggatgaaagaagatgaggcgcggagatatttccaacagcttatacatgctgtggactactgtcatagcagaggggtctaccatagagacctcaagcctgaaaatttactattggactcctatggaaacctcaagatctcagattttggattaagtgctttgtcccaacaagtcagggatgatggactcttgcatacatcgtgtggaacaccaaactacgttgctcctgaggttctcaatgatagaggctatgatggagcaacagctgacatgtggtcatgcggtgttgtactctatgtcctgcttgcaggttacttaccttttgatgattctaatctaatgaatctttataaaaaaatatcatctggtgaattcaactgtcctccgtggctctcactcggtgccatgaaactcatcactagaatcttagatccgaatccgatgactcgtgtaacaccgcaagaggttttcgaagatgaatggttcaagaaagattacaagccacctgttttcgaggagagggatgattcaaacatggacgatattgatgctgttttcaaggactctgaggaacatcttgttactgagaagagagaagaacagccagcggcgatcaatgccttcgagatcatttcaatgtcaaggggacttaacctagagaatctgtttgatccagaacaggaatttaagagggaaacaaggataacattgagaggaggcgcgaatgagatcatcgagaagatagaagaagctgcaaagcctctcggtttcgatgttcaaaagaagaactacaagatgaggcttgagaatgtgaaggctggaagaaaggggaatctcaatgtagcgacagagatattccaagtagcgccaagtcttcatatggttcaagtatcgaagtcgaaaggagacactctcgaatttcacaagttctataagaagctctctaattctctggagcaagtagtctggacgaataacgaagttaagaaagaaacagcaaagtga

SEQ ID NO: 22 - DNA, 264 bp, Arabidopsis thaliana
  misc_feature 1..264: note = ORF of protein Cks1
  source 1..264: mol_type = unassigned DNA
atgggtcagatccaatactccgagaaatacttcgatgacactttcgaatacaggcacgtcgttcttcctcctgaagtcgctaaacttcttccaaagaatcgtcttctctccgaaaacgaatggcgagcgataggagtgcagcaaagccgcggatgggtacattacgcggttcatcgacctgagccgcacataatgctattcaggaggcctcttaactatcagcagcagcaggagaatcaagctcagaacatgcttgttaagtga

SEQ ID NO: 23 - DNA, 1962 bp, Arabidopsis thaliana
  misc_feature 1..1962: note = ORF of protein Cobl8
  source 1..1962: mol_type = unassigned DNA
atgggtttaactcgaaatttcattttatggattcttttgtcatctctcttcacggcgattcaactaacgtcgtctcaacgcaacactccaccaccacgctctaaagacgccgatctctgcaacggcgtgttcgtttcctacacttacttaaccggtaccaagattaaaccaaacgacacgaagaaccagccgtaccggttcgaatcagagatcacggtcctaaacaacggccgcgacgagcttaaatcgtggcaagtctttgttaaattcgcccaccgcgagattcttgtttcagcgaccaacgctgttctctctgacggttctagcctcccggctagcgttgagaacgggacaatcttcgccgggtttccatcggcggatttgaagacggcgatcatgacggccggcgacgtaactcagatggaagcacgtgttgagctcgtgggtactcagtttggagttgctccgccgagtgttccgcttccgaagaacatcactcttgttaatgatggatggtcttgtcctaaacccactcaacaaggtagtaatgttctgcaagtatgttgcacaccgaatccgaacattacaacaagcaaaattggtcaaaagttccttcctaggcaagaaggagatctaaccataatgtacgatgtgaccagagcatatcagtcgagttactcagcgcaggtcactatcgagaatcataacctgcttggtcggcttgacaattgggatctaagttttatgtggatgaaagatgagtttctcttctcaaccaaaggagcttatccaagtgttgtcgattcgtcggattgcatcactggtcctcaagcaaaatactataaagatcttgacttctctaatgtcatgagctgtgcaagaaggccacacatcatagacctacccctgactaagtataacgataccaatgtcgggcgtatcccatactgctgcagaaatggaactattctgccacggtccatggatccagagaagtctaaatcggtttttcaaatagaagtttacaaaatgcctcctgatctcaacatctcctctataacacctccccagagctggcagattaaaggtaaccttaacccggattacaaatgtggccctcctcttcgtgtcagctcaagccagttccctgatacaagcggtttgccttcaaacaagagtgcatttgctagctggcaagtcgtctgcaacatcactcaaccaactcctcctaaatgctgcgtctcattctcttcttatttcaatgactcagtaatcccgtgtaagacctgcgcttgtggaggttgctctagcgacagagtggcgcgtacttgcagcacaacttctccagctcttcctcttccatatcaggctcttctaattccatttgacaaccgaaccaaacttaccaacgcttgggcagtgttaaaaaaccggaaagtcccggacccattaccttgtggagataactgtggagtcagcataaactggcacctagctacagactatcgaggcggatggactgctcgagtcacactcttcaactggggtgatactgatttcgtggattggtttaccgcggttgaactgagaaatgctgcacccggttttcagaaagcgtactcgtttaacggaagtatcatcgcggtcaacgggaagaacacaactgttttaatggaagggctccctggactgaactatctccttgcagagaaagacggaaaaaacccgagtgaagactttcggatacccgggaaacagcagtcggttatctccttcaccaagaaactgactcctggaatcaaagttggttccaaagatgggtttcccaccaaagtactcttcaacggccaggaatgttcactcccttctgtactacccacgagcaacagtcacaggaaacatgtatccacctttcttctcatcctgacaccatttttggctctactgtttcttcggatttaa

SEQ ID NO: 24 - DNA, 1779 bp, Arabidopsis thaliana
  misc_feature 1..1779: note = ORF of protein Coi1
  source 1..1779: mol_type = unassigned DNA
atggaggatcctgatatcaagaggtgtaaattgagctgcgtcgcgacggttgatgatgtcatcgagcaagtcatgacctatataactgacccgaaagatcgcgattcggcttctttggtgtgtcggagatggttcaagattgattccgagacgagagagcatgtgactatggcgctttgctacactgcgacgcctgatcgtcttagccgtcgattcccgaacttgaggtcgctcaagcttaaaggcaagcctagagcagctatgtttaatctgatccctgagaactggggaggttatgttactccttgggttactgagatttctaacaaccttaggcagctcaaatcggtgcacttccgacggatgattgtcagtgacttagatctagatcgtttagctaaagctagagcagatgatcttgagactttgaagctagacaagtgttctggttttactactgatggacttttgagcatcgttacacactgcaggaaaataaaaactttgttaatggaagagagttcttttagtgaaaaggatggtaagtggcttcatgagcttgctcagcacaacacatctcttgaggttttaaacttctacatgacggagtttgccaaaatcagtcccaaagacttggaaaccatagctagaaattgccgctctctggtatctgtgaaggtcggtgactttgagattttggaactagttgggttctttaaggctgcagctaatcttgaagaattttgtggtggctccttgaatgaggatattggaatgcctgagaagtacatgaatctggtttttccccgaaaactatgtcggcttggtctctcttacatgggacctaatgaaatgccaatactatttccattcgcggcccaaatccgaaagctggatttgctttatgcattgctagaaactgaagaccattgtacgcttatccaaaagtgtcctaatttggaagttctcgagacaaggaatgtaatcggagataggggtctagaggtccttgcacagtactgtaagcagttgaagcggctgaggattgaacgcggtgcagatgaacaaggaatggaggacgaagaaggcttagtctcacaaagaggattaatcgctttggctcagggctgccaggagctagaatacatggcggtgtatgtctcagatataactaacgaatctcttgaaagcataggcacatatctgaaaaacctctgtgacttccgccttgtcttactcgaccgggaagaaaggattacagatctgccactggacaacggagtccgatctcttttgattggatgcaagaaactcagacgatttgcattctatctgagacaaggcggcttaaccgacttgggcttaagctacatcggacagtacagtccaaacgtgagatggatgctgctgggttacgtaggtgaatcagatgaaggtttaatggaattctcaagaggctgtccaaatctacagaagctagagatgagaggttgttgcttcagtgagcgagcaatcgctgcagcggttacaaaattgccttcactgagatacttgtgggtacaaggttacagagcatcgatgacgggacaagatctaatgcagatggctagaccgtactggaacatcgagctgattccatcaagaagagtcccggaagtgaatcaacaaggagagataagagagatggagcatccggctcatatattggcttactactctctggctggccagagaacagattgtccaacaactgttagagtcctgaaggagccaatatga

SEQ ID NO: 25 - DNA, 1779 bp, Arabidopsis thaliana; identical to SEQ ID NO: 24
  misc_feature 1..1779: note = ORF of protein Coi1
  source 1..1779: mol_type = unassigned DNA

SEQ ID NO: 26 - DNA, 1590 bp, Arabidopsis thaliana
  misc_feature 1..1590: note = ORF of protein Cpk3
  source 1..1590: mol_type = unassigned DNA
atgggccacagacacagcaagtccaaatcctccgatccgccaccgtcctcctcctcctcttcctccggcaacgtagtccaccatgttaaacctgccggagaacgccgcggatcatccggttccggaaccgtcggatcgtcaggctccggaacaggcggatctcgttccaccacatctactcagcagaatggtcggatcttaggtcggccgatggaggaagtacggagaacctatgagtttggtcgtgagcttggtcgtggccagtttggtgtcacgtacctcgtaactcacaaagagacgaaacaacaggtcgcttgcaagtcaatccctacgcgccgccttgttcacaaagacgacattgaagatgttcgccgtgaagtccagattatgcaccatctcagcggtcaccggaacatagtggacttgaagggagcctacgaggacagacactctgtgaatctgataatggagttgtgtgaaggaggggaattgttcgataggatcatttctaaaggtctttactcagagagagctgctgcggatttgtgtaggcagatggtgatggttgtgcatagttgtcattctatgggtgtaatgcaccgagacttgaagcccgaaaactttctctttcttagtaaagatgagaactcaccattgaaagctacagactttggtctctctgtcttcttcaagccaggtgataagtttaaggatcttgttggaagtgcatactatgttgccccagaagttctaaaacggaactatggaccagaggctgatatctggagtgctggtgtgattctatacatccttctcagtggtgttccgcctttctggggagaaaatgaaacggggatctttgatgccattcttcaagggcaacttgatttttcagctgatccatggccagcactatcagatggtgccaaagatcttgtgaggaaaatgttaaaatatgaccctaaagatcggcttacagctgcggaagtgctaaatcatccgtggattagagaagatggggaggcatcagacaaaccgcttgacaatgcggtgttatccaggatgaaacaattccgggcgatgaacaaactaaaaaaaatggccctgaaggttatcgctgagaacctttctgaagaagaaatcattggtcttaaagagatgttcaaatctctagacactgataacaatggaatagtcaccttggaggaattgagaaccggtctcccaaaacttggttcaaagatctctgaagctgaaataaggcagttaatggaagcagctgatatggatggagatggatcaattgattacttggagtttatatcagccacaatgcatatgaacagaatcgaacgtgaggatcacttgtacaccgcattccagttctttgacaatgacaacagcggctacataacaatggaagagctagagctagccatgaagaaatacaacatgggtgatgataaatccatcaaagagatcattgctgaagtagacaccgatcgtgacggaaaaatcaactacgaagaatttgtagcgatgatgaagaagggaaatccagaattggtgccaaaccgacgcagaatgtga

SEQ ID NO: 27 - DNA, 963 bp, Amaranthus hypochondriacus
  misc_feature 1..963: note = ORF of protein Cpk3
  source 1..963: mol_type = unassigned DNA
atttcacaaaatataagctggttctggccaaccccacaacacccttacctgaccttattcggatgccaaagagaagtcttcagagatcttgtcggaagcgcatattatgtagctcctgaggttttacgtcgtagttatggggctgaagctgatgtttggagtgctggagtgatattatacatcctacttagtggagttccaccattttggggagagaatgagcagggaatttttgattctattctgcggggcaatcttgatttcaattcagatccatggctctcaatatctagcagtgccaaagacctggtgacaaagatgttgcggcaggatcctaaggaaagactttctgctcgtgatgctttaaatcatccttggatgagagaggatggagaagctcctgataagcctattgatattgctgttttgactagaatgaagcagtttagggcaatgaacaaactaaaaaaagtagcactaagggtaattgccgagaacctttctgaagaaaaaataattggtttaaaagaaatgtttaaatccatagatactgataatagtggaactatcacctatgaagaattaaagagcggtcttccaaagctgggaactagactttctgagaaagaagtcaagcagttaatggaggcggctgatgttgatggtaatggaaccatagattacatggaattcatcacagctacaatgcatatgaataggatggaggaaggggaccatatatacaaagcctttgaatattttgatgtggataaaagcgggtacattaccatggaggaattggcgcaagccttgaagaagtataatatgggtgatgcaaaaacaatacgggagataattgcagaagttgatacagacaatgatggtaaaatcaattatgatgaatttgtagcaatgatgaagaaaggcaaccctgatatggttaccaacagacggaagagatga

SEQ ID NO: 28 - DNA, 1647 bp, Brachypodium distachyon
  misc_feature 1..1647: note = ORF of protein Cpk3
  source 1..1647: mol_type = unassigned DNA
atgggcgcccgcgcctcccgccaccgccaccaatccccctcccaatcccaatccagcgacgactcccagtcccaccacaagccacgacccaagcccaagcccaagcatcaccaccaaccaccacaaccccctccgcggcatcagccccgccaccaccaccacccacccccgcaacgccctccgccgcaacatcaacaacaacaccagcagcagcaccatgcggcggcggcggcggaggacacgggccgggtcctgggccgcccgctgtcggacgtgcgcgcgacctacacgttcggccgggagctgggccggggccagttcggggtcacctacctagcaacccacaagtccaccggcgcgcgctacgcctgcaagtcgatctccgcccggaagctgtcccgcggggacgacgccgacgacgtccgccgcgaggtgcagatcatgcaccacctcacgggccaccgcagcatcgtcgagctcaagggcgcccacgaggaccgccactcggtcaacctcgtcatggagctctgcgagggcggggagctcttcgaccgcatcatcgcccggggacactactccgagagcgccgcggcggcggtctgcagggaggtggttaatgtcgtccactgctgccattccatgggggtcatgcatcgggacctcaagccggagaactttttgtttctgaataagcgggaggactcgccgcttaaggctactgattttggtctctccgtcttcttcaagcccggggagcagttcagggatctcgttgggagcgcctactatgtggctccggaggtgctaaagcggcgatatggagctgaggcggacatatggagtgctggagtcatcctctacattctcctctccggtgtccctcctttctgggcagagaatgaggatggtatattcgatgctgttctgcaaggtcatattgatttctcatctgatccctggccatcaatttcacacggggctaaagacttggtcaggaggatgctgcggcaggaccccaaggagcgcctcactgctgctgaaattttgaaccatccatggattagagaggacggagaggccccagataaaccacttgatattacagtgatcagtagaatgaagcagttcagagcaatgaacaaacttaagaaggttgccttgaagattgttgcagaaagcttgtcagaagaagagatcgtgggcttaaaacaaatgttcaaatccctggatactgacaacagcgggacaattactcttgaagaactacgggctggattaccaaagctcggtaccaaaattactgaatcagaaataagacagttgatggaggcagctgatgttgatggaaatgggaccattgattatgttgaattcatatcggcaacgatgcacatgaatagactagagaaggaggatcacatatttaaagcatttgaatattttgacaaggaccacagtggctacataacagttgatgagttggaagaagccttgaagaagtatgatatgggtgatgaggcaacaatcaaggaaatcattgctgaagtggatacagatcatgatgggaaaataaactaccaagagtttgttgccatgatgaaaaacaatagcccagagattgttccaaatcgacgccgcttgttttaa

SEQ ID NO: 29 - DNA, 1581 bp, Brachypodium distachyon
  misc_feature 1..1581: note = ORF of protein Cpk3
  source 1..1581: mol_type = unassigned DNA
atgggcaaccgcacctcgcggcaccgccgcgccgccgccgccgagcagccgacgccggttccgtccccgccgccggcccagcccaaacccgagccgcaacagcagacgctccactggccgcagccgaagccccagccgccccccgccccggcggtggaagcgggcgtggtggcgatggggcgcgtgctggggcggccgatggaggacgtgcgcgcgacctacaccttcggccgcgagctcggccggggccagttcggggtcacctacctcgtcacgcacaaggccacgggccagcgcttcgcctgcaagtccatcgccacgcggaagctcgtccaccgcgacgacatcgaggacgtgcagcgggaggtgcagatcatgcaccacctcacgggccaccgcaacatcgtcgagctccgcggcgcctacgaggaccgccactcggtcaacctcgtcatggagctctgcgagggcggggagctgttcgaccgcatcatcgcgcggggccactactccgagcgagccgccgccgtgctctgccgcgagatggtctccgtcgtgcacagctgccactcgatgggggttttccatcgggatctcaagcctgagaactttctgtttctgaataacaaggaagactcgccactcaaggccactgactttggcctctccgtcttcttcaagcatggggagcagtttaaggatcttgttggaagcgcatattatgttgctcctgaggtgctgaaacggcactatggagcagaagctgacatatggagtgcaggggttattctttacatccttctgtctggtgttcctcctttttgggcagataatgaggatggcatatttaaggctgttttgcttggtcacattgatttctcgtctgacccctggccttcaatttctaatcccgcgaaagatttggtcaagaagatgctgcgacaagaccccaaagagcgcttaactgctgcagagattttagaccacccatggattaaggaagatggagaggccccggataagccacttgacattacagttatcagtagaatgaagcagttcagggcgatgaacaagcttaagaaagttgcactgaagattgttgcagagaacttgtctgaggaagagattacaggcttgaaagaaatgttcagatccctggatactgataacagcgggacaattactcttgaagagctacgatctggtttaccaaaacttggcaccaaaatatctgaatcagaaattacacaactgatggaggcggctgatgttgatggaaatgggaccattgattattctgagtttgtatcggcgacaatgcacatgaatagattggagaaggaagaccacatacttaaagcatttgaatactttgataaggaccacagcggatacataacagtagatgagttggaagaagctctgaagaaatatgacatgggggacgataaaacaattaaagaaatcattgctgaagtagatacagataatgacggaagaatcaactaccaggagttcgttgctatgatgagaaacaacagccctgagattgttccaaatcggagacgcatgttttaa

SEQ ID NO: 30 - DNA, 1314 bp, Glycine max
  misc_feature 1..1314: note = ORF of protein Cpk3
  source 1..1314: mol_type = unassigned DNA
atgggcaactgcagcggcggcggcggaatcggcagcaccacctactccgacgaccatccccgccgcaacggcatcaccgtcctccctccgaactcgaacctttcagccccaccagcgacctcctcgctgggccggcgcatggaggacatgcgatccatttacatattcgggcgggagctggggcgcgggcagttcggcgtgacgtacctggttacccacaaggccacgaaggagcagttcgcgtgcaagtcaatcgccacgcggaagctggtgaaccgcgacgacatcgacgacatccggcgcgaggtgcagatcatgcaccacctcaccggccaccgcaacatcgtcgagctcaagggcgcctacgaggaccgccactccgtcaacctcgtcatggagctctgcgccggcggcgagctcttcgaccgcatcatcaccaagagccactactccgagcgcgccgccgccaactcctgccgccagatcgtcaccgtcgtccataactgccactccatgggagtcatgcatagggacttgacgagaatttcttgttgctcaaccataacgatgattcacccctcaaggccacggattttggtctctccgtcttttttaagccaggtgctgagaagaagttacgggcctgaaactgatatttggagtgcaggagttatattgtacattttgctttctggtgttccacctttttgggcagaaaatgaacagggtatctttgatgctattcttcgtggacatattgatttcgcatcagatccttggccttccatatcaagtagtgccaaagatctggtcaagaagatgctacgagctgaccctaaagaacggctttcagcagttgaagtacttaatcatccctggatgagagtggatggagatgcatctgataagcctcttgatattgctgttttaaccagaatgaaacatttccgggcaatgaacaagctaaaaaaagtagccctgaaggttattgcggaaaacctttctgaagaagaaattattggcttgaaggaaatgttcaaatccatggacacagataacagtggaacaatcacatttgaagagttgaaagctggtctcccaaaattgggtagtaaactttctgaatctgaagttaggcagttgatggaagcgggtctgattttagcctaccttataaatacctttccctgccttgtactctctctccaacaaacccgcatgcacttccccaaccccatgatcaggtgcaatgcaccttttgcctctcattacatacaccttagtgatctttggctgtggggccacatcacttaa

SEQ ID NO: 31 - DNA, 1557 bp, Glycine max
  misc_feature 1..1557: note = ORF of protein Cpk3
  source 1..1557: mol_type = unassigned DNA
atgggaaactgcagcagcggggccggagcccccaccacctactccgacgaccatccccaccacaacggcataaccgtcctccctcccaactcaaacccttcaccccaactccccaagcccccaccgacctcctcctcctcctcctcgctgggccgggttttgggccggcccatggaggatgtccggtccatttacatattcgggcgggagctggggcgcgggcagttcggcgtgacgtacctggtgacccacaaggccacgaaggagcagttcgcgtgcaagtcaatcgcgacgcggaagctggtcaaccgcgacgacatcgacgacatccgccgcgaggtccagatcatgcaccacctcaccggccaccgcaacatcgtcgagctcaagggcgcctacgaggaccgccactccgtcaacctcgtcatggagctctgcgccggcggtgagctcttcgaccgcatcatcaccaagggccactactccgagcgcgccgccgccaactcctgccgccagatcgtcaccgtcgtccataactgccactccatgggagtcatgcatagggacttgaagcccgagaatttcttgttgctcaacaagaacgatgattcccccctcaaggccacagattttggcctctccgtcttttttaagccaggggatgtgtttagagaccttgttgggagtgcttactatgttgctcctgaggtgctgagaagaagttatggacctgaagctgatatttggagtgctggagttatattgtacattctactttctggtgttccacctttttgggcagaaaatgaacagggtatctttgatgctattcttcgtggacatattgattttgcatcagatccttggccttccatatcaagtagtgccaaagatctggtcaagaagatgctacgagctgaccctaaagagcgactttcagcggttgaagtacttaatcatccctggatgagagtggatggagatgcacctgataagcctcttgatattgctgttttaaccagaatgaaacaattccgagcaatgaacaagctaaaaaaagtagctctgaaggttattgctgaaaacctttctgaagaagaaattattggcttgaaagaaatgttcaaatccatggacacagataacagtggaacaatcacattcgaagagttgaaagctggtctcccaaaattgggtactaaactttccgaatctgaagttaggcagttgatggaagcggctgatgtagatggaaatggaaccattgattacattgaatttataactgctaccatgcacatgaatagaatggaaagagaggatcacctatataaagcctttgaatattttgacaatgataagagcgggtacatcacaatggaagagttggaatctgcccttaagaaatataatatgggtgatgagaaaacaataaaggagatcattgctgaagttgatacagacaatgatggaagaattaactatgatgagtttgtagccatgatgaggaaaggcaacccagacataacgcacataacccaaagacgtcgcaaataa

SEQ ID NO: 32 - DNA, 1518 bp, Glycine max
  misc_feature 1..1518: note = ORF of protein Cpk3
  source 1..1518: mol_type = unassigned DNA
atggggaactgtaacagcgaaccctcttcccaaacccagccacattccgatcaccaccatcatcacccccaaccctccaacggcatcactgtccttcctcccaattcgaagccctccgtaggccgggttctgggccggcccatggaggacgcccgctccacctacaccttcggccgcgagctgggccgcggccaattcggcgtgacctaccaagtgacccacaaacacacgaaacagcaattcgcgtgcaaatccatcgccacgcgcaagctcgtccaccgcgatgacttggaggacgtccggcgagaggtgcagatcatgcaccacctcaccggccaccgcaacatcgtggagctcaagggcgcttacgaggaccgccactccgtcaacctcatcatggagctctgcggcggcggcgagctcttcgaccgcatcatcgccaagggccactactccgagcgcgccgccgcagatctctgccgccagatcgtgacggtggtccacgattgccacaccatgggagtgatgcacagagatctcaagccggagaacttcctgttcctcagcaaggacgagaactcgccgcttaaggccacggacttcggtctctctgtcttcttcaagcccggcgatgtgtttaaggatcttgtcgggagtgcgtactatgttgctcctgaggtgctgcgcagaagctatggacccggagctgatatttggagtgctggcgttatattgttcattttgctctctggtgtccctcccttctggtcagaaaaggaacagggtatatttgatgctattcttcgtggccatattgatttcgcatcagatccttggccttccatatcgagtagtgccaaagatctggttaagaagatgttacgagctgaccccaaacaaagactctcagcagttgaagttcttaatcatccttggatgagagaagatggagcatctgataaacctctagatgttgctgttttatccagaatgaaacaatttagggcaatgaacaagctaaagaaagttgctctgaaggttattgctgaaaacctttctgaagaagaaatcattggtttgaaggaaatgtttaaatccatggacacagataacagtggaacaatcacttttgaagagttgaaagctggtctgccaaaactgggtactaaggtttctgagtctgaagtaaggcaattgatggaagcggctgatgtggacggaaatgggaccattgattacattgaatttataacagctaccatgcacatgaatagaatggaaagagaggaccacctatataaagcttttgaatattttgacaaggatagaagcgggtacatcacagtggaagagttggagtctgcccttaaaaaatataatatgggtgatgagaaaacaataaaggaaatcattgccgaagttgatgcagacaatgatggaagaattaactatgacgagtttgtagccatgatgaggaaaggcaatccagatttagtaaacaacagacgtcgcaaataa

SEQ ID NO: 33 - DNA, 1587 bp, Glycine max
  misc_feature 1..1587: note = ORF of protein Cpk3
  source 1..1587: mol_type = unassigned DNA
atggtttcacgtttcagtcctgatcgagtaaacaccaagaagcagcagcgcagtaacgacaccgtttcgatggggaactgtaacagcgaagcctcttcccaaactcagccacattccgatcaccatccccaaccacgctccaactccaacggcatcaccgtccttcctcccaattcgaagccctccgttggccgggtcttgggccggcccatggaggacgtccgctccacctacaccttcggccgcgaactgggccgcggccaattcggcgtgacataccttgtgacccacaaacacaccaaacaacaattcgcgtgcaaatccatcgccacgcccaaactcgtccaccgcgacgacttggaggatgtccggcgagaggtgcagatcatgcaccacctcaccggccaccgcaacatcgtggagctcaagggcgcttacgaggaccgccactccgttaacctcatcatggagctctgcggcggcggcgagctcttcgaccggatcattgccaagggccactactccgagcgcgccgccgccgacctctgccgccagatcgtgacggtggtccacgattgccacaccatgggagtgatgcacagggatctcaagccagagaacttcctgttcctcagcaaggacgagaactcgccgcttaaggccacggacttcggtctctccgtgttcttcaagcccggcgacgtgtttaaggatcttgtcgggagtgcgtactatgttgctcctgaggtgctgcgtagaagctatggacccgaagctgatatttggagtgctggggttatattgttcattttgctctccggtgtccctcccttctggtcaaaaaaggaacaaggtatctttgatgctattcttcgtggccatattgatttcacatcagatccttggccttccatatcgagtagtgccaaagatctggtcaagaagatgttacgagctgaccccaaacaaaggctctcagcagttgaagtccttgatcatccttggatgagagaagatggagcatctgataagcctctagatgttgctgttttatccagaatgaaacaattcagggcaatgaacaagctaaagaaagttgctctgaaggttattgctgaaaacctttctgaagaagaaatcattggcttgaaggaaatgtttaaatccatggacacagataacagtggaacaatcacttttgaagagttgaaagctggtctgccaaaactgggtactaaggtttctgagtctgaagtaaggcagttgatggaagcggctgatgtagacggaaatgggaccattgattacattgaatttataacagctaccatgcacatgaatagaatggaaagagaggaccacctatataaagcttttgaatattttgacaaggatagaagcgggtacatcacaatggaagagttggaatctacccttaaaaaatataatatgggtgatgagaaaacaataaaggaaatcattgtggaagtcgatacagacaatgatggaagaattaattatgatgagtttgtagccatgatgaggaaaggcaaaccagatttagtaaccaacagacgtcgcaaatga

SEQ ID NO: 34 - DNA, 1698 bp, Oryza sativa
  misc_feature 1..1698: note = ORF of protein Cpk3
  source 1..1698: mol_type = unassigned DNA
atggtctcctcctcctcctccccccaatccaaacccccaaaaccaaagccaaaacccctctcgccaccgatgggcgcccgcgcctctcgccaccgccaatcccccgaccaatcccaatcccaatccccatccccacaccacaagcaccaccaccaccaccaaaccacccgggcccccaagcccaagcccaagccccagccaccgccgccgcagcagccgcgatctcagcctccgccgccgccgcggcaccagccccagcaggcgccccagcaggcggcggcggaggatggggtggggcgggtgctggggcggcccatggaggacgtccgtgcgacctacaccttcgggcgggagctggggcgggggcagttcggggtgacctacctcgccacccacaagcccaccggccgacgctacgcctgcaagtccatcgccgcccgcaagctcgcccgccccgacgacctcgacgacgtccgccgcgaggtccacatcatgcaccacctcaccggccaccgcaacatcgtcgagctgcgcggcgcctacgaggaccgccactccgtcaacctcgtcatggagctctgcgagggcggcgagctcttcgaccgcatcatcgccaggggccactactccgagcgcgccgccgccgccctctgcagggagatcgtctccgtcgtccacagctgccactccatgggggtcatgcacagggacctcaagcccgagaacttcctcttcctcaacaagcgcgaggactccccgctcaaagccaccgattttggcctctccgtcttcttcaagcccggtgagcagttcagagatcttgttggaagtgcatattatgtggctcctgaggtcctaaaacgactatatggagctgaggcagacatatggagtgctggagttatcctttacatccttctatcaggggttcctccattctgggcagaaaacgaggacggtatatttgatgctgttctgcaaggtcatatcgacttctcatctgaaccatggccttctatatctagtggtgcaaaagacttggtcaagcggatgcttcggcaggacccaaaggagcggttaactgctgctgaaattttgaaccacccatggattagagaggatggagaggccccagataaaccacttgatattacagtgatcagtagaatgaagcagttcagagcaatgaacaaacttaagaaggttgccttgaaggtcgttgcagagaacttgtcagaggaagagattgtgggtttaaaggaaatgttcaaatctttagatactgataacagtgggacgataactcttgaagaactaagagctggtctaccaaagcttggcactaaaatttcggaatcagaattaaggcagttgatggaagcggctgatgttgatggaaatgggtccattgattatgttgaatttatatcagcaacgatgcacatgaatagattagagaaggaagatcacatatataaagcatttgaatattttgacaaggaccacagcgggttcataacagtcgatgaattggaagaagctctgacaaagtacgacatgggtgatgaagcgacaattaaagaaataattgctgaagtggatacagaccatgatggaagaattaactaccaggagtttgttgccatgatgaagaacaacagccctgagattgttccaaatcgacggcggatgttttaa

SEQ ID NO: 35 - DNA, 1557 bp, Oryza sativa
  misc_feature 1..1557: note = ORF of protein Cpk3
  source 1..1557: mol_type = unassigned DNA
atgggcaaccgcacgtcgcgccaccaccgcgccgctcccgagcagccgccgccccagcccaagccgaagcctcagccgcagcagcagcagcagcagtggccgaggccgcagcagccgaccccgccgccggcggcggcgccggacgcggcgatggggagggtgctggggcggccgatggaggacgtccgcgcgacctacaccttcgggcgcgagctcgggcggggccagttcggggtcacctacctcgtcacccacaaggccacggggaagcgcttcgcctgcaagtccatcgccacgcggaagctcgcccaccgcgacgacatcgaggacgtgcgccgggaggtgcagatcatgcaccacctcacgggccaccgcaacatcgtcgagctccggggcgcctacgaggaccgccactcggtcaacctcatcatggagctctgcgagggcggggagctcttcgaccgcatcatcgccagggggcactactccgagcgcgccgccgccgcgctctgccgcgagatcgtcgccgtcgtgcacagctgccactccatgggggtcttccaccgggatctcaagccggagaactttttgttcctcagtaaaagcgaggactcgccgctcaaggccactgacttcggtctttccgttttcttcaagcctggggagcattttaaggaccttgttggaagtgcgtattatgttgcacctgaggtgctgaaacgaaactatggagcagaggctgacatatggagtgctggcgttattctttacatccttctttctggtgttccacctttttgggcagagagtgaagatggtatatttgatgccgtattacgtggtcacattgatttctcgtctgaaccttggccttcgatatccaatggtgcaaaagatttggttaagaagatgttgcggcaagacccaaaagagcgccttacttctgctgaaattttgaaccacccatggattagagaggatggagaagccccagacaagccacttgacattacagttatcagcaggatgaagcagttcagggcaatgaacaagcttaagaaagttgcattgaaggttgttgcagagaacttgtcagatgaagagattacaggcttaaaagaaatgttcagatccttggatactgataacagtgggacaataactcttgaggagctaagatctggtttaccaaaacttggcaccaaaatttctgaatcagaaattagacagctgatggaggcggctgatgttgatggaaatggaaccattgactatgctgagttcatatcagcgacaatgcacatgaacagattggagaaggaagaccacatacttaaagcatttgagtattttgataaggaccacagcggatacataacagtagatgagttggaagaagctctgaagaagtatgatatgggggacgataaaacgattaaggaaatcatcgctgaagtagatactgatcatgatggaagaattaactaccaggagtttgtcgccatgatgaggaacaacaaccctgagattgctcccaaccggcggcgcatgttctaa

SEQ ID NO: 36 - DNA, 1551 bp, Solanum lycopersicum
  misc_feature 1..1551: note = ORF of protein Cpk3
  source 1..1551: mol_type = unassigned DNA
atgggcaactgtaacagcttatcttccgaccaagccaccaccacttccggcggcggcggaggcggcggcgctccttcaacgccgtcaaccaccggtatcagaatcatcccttcctcacaaccaccgcctccacgaccactctccggcatcggccgtgttctcggccggccaatggaggatgtccggtccatctacattttcggcggcgaactcggccggggtcaattcggagtcacctacttagttactaacaaaaaaacgcgccagcagttcgcgtgcaagtcaatcgctacacggaaactcatcaacaaggacgatgtcgacgacgtccgccgtgaagttcagatcatgcatcaccttaccggtcaccggaatatcgttgagcttaaaggaactttcgaggataaacaccatgttcatctcgtcatggaactttgtgccggcggcgagctttttgaccggataattgctaaaggacactattccgagcgagctgctgctggagtttgccggcagatggtaactgttgttcacaattgtcattctatgggagttatgcatagggatttaaaacctgaaaatttcctttttttgagctccgatgaaacttcaccattaaaggctactgattttggactatcagtctttttcaaaccaggtgatgtgttcaaagacctggttggtagtgcatattatgttgctcctgaagttctgcgccgagaatatggacctgaagctgatatttggagtgctggagttattctgtacattctactcagtggtgttccaccattttatggagagaatgatcagagtatctttgatgccgttcttcgaggtcatcttgatttttcatctgatccttggccttcagtatcgagtagcgctaaagatcttgttaagaagatgctgcgatctgatcctagagagaggatttctgcagctgaagtcctaaatcatccatggatgagagaagatggtgatgcatctgacaaacctcttgacattgctgtcttatctagaatgaaacaattccgtgctatgaacaagctgaagaaagtagcactgaaggtaattgctgaaaatctttctgaagaagaaatcattggcttgaaggaaatgttcaaatctatagacacagatgacagtgggacaattacatttgaagaattgaaagccggtcttacaaaaatgggtacaaagctttcagagtcggaagtcaggcagttgatggaagcggctgatgttgatggaaatggaacaattgattaccttgagttcataacagctacgatgcacatgaatcgcatggaaagagaagatcacttgtacaaagcctttgagtattttgataaggataagagcgggtatattacaatggaagaattggagcatgcgctgaaggaatacaacattactgatgacaaaacaataaaagagatcattgctgaagttgacacagacaatgatggaagaatcaattacgatgagttcgctgccatgatgaggaaaggtaatccagattttgtcaacaataggcgccgcagatga

SEQ ID NO: 37 - DNA, 1620 bp, Zea mays
  misc_feature 1..1620: note = ORF of protein Cpk3
  source 1..1620: mol_type = unassigned DNA
atgggcggccgcgcctcccgccaccgcgcgcagccgcacgaccagtcttctcaccctcctcctcggccgccgcccaagcagcagcagggccagcagcaccccaacaaccgccccaacaagcccaagcagcagcagcagcccccacctccacgtcctccgccgccacaccaggctccccccgccgcctcccccgccgccccgggcgcggccaccgtcggccgcgtgctgggccggccgatggaggacgtgcgcgcgtcctacacgttcggccgggagctgggccggggccagttcggcgtcacctacctggccacgcacaagcccaccggccgccggtacgcgtgcaagtccatcgcgacgcggaagctcgcgcacagcgacgacgtcgacgacgtccgccgggaggtgcagatcatgcaccacctcacgggccaccgcagcatcgtcgagctccggggcgcctacgaggaccgccactccgtcaacctcgtcatggagctgtgcgagggtggggagctcttcgaccgcatcatcgcgagggggcactactccgagcgtgccgccgccgccctctgcagggagatcgtctccgtcgtccacagctgccactccatgggggtcatgcatcgggacctcaagcccgaaaacttcctcttccttaacaagagggaggactcgccgctcaaggccacggatttcggactctccgtcttcttcaagcccggcgagcagttcagggatcttgtcggcagcgcttactacgtcgccccagaggtgctcaagcggcgatacggagctgaggcagacatatggagtgccggagttatcctctacatcctgctatccggcgtccctcctttctgggccgagaacgaggatggcatatttgatgctgttttgcgaggtcatattgacttcgcatctgatccctggccttccatatcgaatagcgcaaaagatttggtcaagaagatgctgcgccaagaccccaaggagcggcttactgctgctgaaattttgaaccatccatggattagagaggatggagaggccccagacaagccactagacataacagtaataagcagaatgaagcagttccgggccatgaacaagcttaagaaggttgccttgaaggttgttgctgagaacttgtcagaggaagagatcgtgggcttgaaggaaatgttcaaatccctggatactgacaacagtgggacaataacgctggaagaattgagagctggtttaccaaagctcggcaccaaaatctctgaatcagaaataaggcagttgatggaagcagctgatgttgatgggaatggcaccattgattatgttgaatttatatcagcaacaatgcacatgaatagactagagaaggaggatcatatatttaaagcattcgagtattttgacaaggatcatagcgggcacattacagttgatgagttggaagaagctttgaagaagtatgatatgggcgatgaggcaacagttaaagaaattattgctgaagtagacacagatcacgatgggagaatcaactaccaggagtttgttgccatgatgaagaataatagccccgagattgttccaaatcggaggcgcatgttttaa

SEQ ID NO: 38 - DNA, 1593 bp, Zea mays
  misc_feature 1..1593: note = ORF of protein Cpk3
  source 1..1593: mol_type = unassigned DNA
atgggcaaccgtgcgtcgcgccaccaccgccaagccgccgagcagcaaccggcgacgccgaccctgccgaagccccagccccagatccagccgccgcctccgcctccgccgacgcagacgcagcagtaccagcaggcgccgccgcagtcgcagcagaagccgaagccggcggcggacgtgggggcggtggggcgcgtgctggggcggcccatggaggacgtgcgcgcgacctacactttcggccgcgagctcgggcggggccagttcggggtcacctacctcgtcacgcactcggagaccgggcagcgcttcgcctgcaagtccatcgccacgcggaagctcgtccaccgcgacgacattgaggacgtgcgccgggaggtgcagatcatgcaccacctcacgggccaccgcaacatcgtcgagctccggggagcatacgaggacaggcactcggtcaaccttgttatggagctctgcgagggcggggagctcttcgaccgcatcatagccaagggccactacaccgagcgagccgccgcctcgctctgccgcgagatcgtcgccgtcgtccacagctgccactccatgggggtcttccatcgggatctcaagcccgagaacttcctgttcctcaataacaaggaggactccccgctcaaggccacggacttcggtctctccgtcttcttcaagcacggggagacgtttaaggatcttgtgggaagtgcatattatgttgctcctgaggtactgaaacggaaatatggggcagaggctgacatatggagtgctggggtcattctttacatccttctgtctggtgttcctcctttttgggcagagaatgaggatggcatatttgatgctgtcttgcgcggtcacatcgatttctcttctgacccttggccttcaatatccaatggtgcaaaggatttggttaagaagatgctgcgacaagacccaaaagaacgcctgactgctgctgaaattttgaaccacccatggattagagaggatggagaggccccagataagccacttgacattactgtcattggtagaatgaaacagttcagggcaatgaacaagctcaagaaagttgcattgaaggttgtggctgagaacttatcagatgaagagattatgggcctaaaagaaatgttcagatccctggatacagataacagtgggacaattactcttgaagagctaagatctggtttaccaaaacttggtacaaaaatttctgaatcagaaattaaacaattgatggaggcggctgatgttgatggaaatggtaccattgattatgcggagtttatatcagccacaatgcatttgaatagattggagaaggaagaccacatactcaaagcttttgaatatttcgataaggatcacagcggatacataaccgtagatgagttggaagaagctttgaagaagtatgatatgggagatgacaaaactataaaagaaatcattgctgaagtagatacagatcatgatggaagaattaattaccaggagtttgttgccatgatgaggaacaacagccctgagattgttccaaatcggaggcgcatgttctaa

SEQ ID NO: 39 - DNA, 1575 bp, Zea mays
  misc_feature 1..1575: note = ORF of protein Cpk3
  source 1..1575: mol_type = unassigned DNA
atgggcaaccgtgcgtcgcgccaccaccgccacgccaccgagcagcagccagagacgccgacccttcccaagccccagatccagccgccgccgccgccgacgcagacgcagcagtaccagcaggccccgccgccgccgaagccgaagccggcggcggacgcgagggcggtggggcgcgtgctggggcggcccatggaggacgtcaaggcgacctacaccttcggccgcgagctcgggcggggccagttcggggtcacctacctcgtcacgcaccgggagactgggcagcgcttcgcctgcaagtccatagccacgcggaagctcgtccaccgcgacgacatcgaggacgtgcgccgggaggtgcagatcatgcaccacctcaccggccaccggaacatcgtcgagctccggggagcctacgaggacaggcgctcggtcaacctcgctatggagctctgcgagggcggggagctcttcgaccgcatcattgccaggggccactacaccgagcgcgctgccgccgcgctttgccgggagatcgtcgccgtcgtccacagctgccactccatgggggtcttccaccgggacctcaagcccgagaacttcctgttcctcaataacaaggaggactccccgctcaaggccacggacttcggcctctccgtcttcttcaagcccggggacacgtttaaggatcttgtgggaagtgcatattatgttgctcccgaggtattgaagcggcactatggggcagaggctgacatatggagtgctggggttattctttacatccttttatctggtgttcctcctttttgggcagagaatgaggatggcatatttgatgctgtcttgcacagtcacattgatttctcttctgatccttggccttcaatatctaatggtgcaaaagatttggttaagaagatgctgcgacaagacccaaaagaacgcctgactgctgctgaaattttgaaccacccatggattagagaggatggagaggccccagataagccacttgacattacggtcatcagtagaatgaaacagttcagggcaatgaacaagctcaagaaagttgcattgaaggttgtggcagagaacttatcagatgaagagattatgggcctaaaagaaatgttcagatccctggatactgataacagtgggacaattactcttgaagagctaagatctggtttaccaaaacttggtactaaaatttctgaatcagaaatcaaacagttgatggaggcggctgatgttgatggaaatggtaccattgattacgcggagttcatatcagccacaatgcatttgaatagattggagaaggaagaccgtatactcaaagcttttgaatattttgataaggaccatagcgggtacataacagtagatgagttggaagaagctctgaagaagtatgatatgggagatgataaaacaataaaagagatcattgctgaagtagatacagatcatgatggaagaattaattaccaggagtttgttgccatgatgaggaacaatacccctgagattgttccaaaccggagacgcatgttctaa

SEQ ID NO: 40 - DNA, 1599 bp, Brassica rapa
  misc_feature 1..1599: note = ORF of protein Cpk3
  source 1..1599: mol_type = unassigned DNA
atgggacacagacacagcaagtccaaatcctccggtccgccaccgtcctcctcctcctcctcttcctccaccaacgtcgtccaccatgtccaaccttccggagaacgccgcggatcttccggttctggccccacacccgtcggatcttcctcaaccggcggatcccgctccgccgcatctgctcagcagaacggtcggatcttaggccgtccgatggagaatgtcaggggaacgtacgacttcggcaaggagcttggtcgtggccagttcggagtcacgtatctcgtcactcacaaagagaccaaaaaactcttcgcctgcaagtcgatccccacgcgccgcctcgtccaccgtgacgacattgaagacgtccgccgtgaagtccagattatgcaccatctcagtggtcacaggaacatagtggacttgaagggagcttacgaggacagacactcggtgaatctgataatggagctgtgcgaaggaggggagcttttcgacaggatcatagcgaaaggccattacacggagagagccgctgcggatttgtgcaggcagatggtgatggttgtgcatagttgccactctatgggtgtgatgcaccgtgatttgaagcctgagaactttctctttctcagcaaagatgagaactctccgttgaaagctacagactttggtctatccgtcttctttaaaccaggagataagtttaaggatcttgttgggagtgcatactatgtggccccggaggttttgaaacggaactatggaccagaggctgatatctggagtgccggtgtgattctcttcatccttatcagcggtgttccgccgttttggggagaaaatgagacagggatctttgatgctattctaaaaggagagcttgacttctcagctgatccgtggccaacggtgtcggctggtgccaaagatcttgtgaagaaaatgttgaagtatgatcctaaagaccggcttacagctagcgaagtgctgaaccatccgtggattaaagaagatggagaggcctcagacaaaccgcttgacaatgctgtgttgtccaggatgaaacagttcagagccatgaacaaactaaagaaaatggcactgaatgtaattgcagagaacctgtctgaagaagaaatcattggtctgaaagagatgttcaaagctctagacaccgataaaaacggaatagtcaccttggaggaattgagaaccggtctcccaaagcttggcaataagatctctgaggctgaaataaaacagttaatggaagcagctgatatggatggagatggatcgatagattacttggagtttatatcagcgacaatgcatatgaacagaatcgaacgtgaggatcatttgtacactgctttccagtacttcgacaaggacaacagcgggtacataacaatggaagagctagagcaggccatgaagaaatacaacatgggtgatgacaaatccattaaagagatcattgctgaagttgacaccgatcgtgacgggaaaataaactacgaagagttcgtagcgatgatgaagaagggaaatccagaactggtgacgaaccgacgcagagtcaacatgtaa

SEQ ID NO: 41 - DNA, 1578 bp, Brassica rapa
  misc_feature 1..1578: note = ORF of protein Cpk3
  source 1..1578: mol_type = unassigned DNA
atgggacacagacacagcaagtccaagacctcctcctcctcctcttcctccgacaacgtcgtccaccatgtcaaaccttccggcgaacgccgcggatcctccggctccggttccggacccgtcaaatcctcctcaggctccggaaccggcggatcccgttctcaacagaacggtcggatcctaggcaagccgatggaggatgtcaagggaacctacgacttgggtcgcgagcttggtcgtgggcagttcggagtcacgcacctcgtcactcacaaagagaccaagaaagtcttcgcttgcaagtcaatccccacgcgccgactcgtccaaagtgacgacattgaagatgttcgccgtgaagtccagattatgcaccatctcagcggtcacaggaacatagtggacttgaagggagcttacgaggacagacagtcggtgaatctgatcatggagctgtgtgaaggaggggagcttttcgataggatcatagctaaaggtcattacacagagagagccgctgcggatttgtgcaggcagatggtgatggttgtgcacagctgccactctatgggcgtgatgcaccgagacttgaagcccgaaaacttcctctttctgagcaaagatgagagctccccgttgaaagctacagactttggcctctccgtcttcttcaaaccaggagataagtttaaggatcttgttggaagtgcatactatgttgccccggaagttttaaaacggaactatggaccagaggctgatatctggagtgccggtgtgattctgtacatccttctcagtggtgtcccgccattttggggagaaaatgagacagggatcttcgatgccattctagaaggaaagcttgatttttcagctgatccatggcctgatgtatccaacggtgccaaagatcttgtgaagaagatgttgacgtatgaccctaaagaccggcttacagcgagtgaagtgctaaaccatccgtggattaaagaagacggagaggcgtcagacaaaccgcttgacaatgctgtgttatccaggatgaaacagttccgagcgatgaacaaactcaagaaaatggcactcaaggtcattgcagagaacttatctgaagaagaaatcattggtctgaaagagatgttcaaagctctagacactgataaaaacggaatagtcaccttggaggagttgagaaccggtctccccaagcttgggtctaagatctcagaggctgagataaaacagttaatggaagcagctgatatggatggagatggatcaatagattacttggagtttatatcagcgacaatgcatatgaacagaatcgaacgtgaggatcacttgtacactgctttccaatacttcgacaaggacaacagcggctacataacaatggaagagctagaacaagccatgaagaaatacaacatgggtgatgacaaatccatcaaagagatcattgctgaagtagacaccgatcgtgacggaaaaatcaattacgaagaattcgtggcgatgatgaagaaaggacatccagagttggtgacaaaccgacgcagagtcaatatgtaa

SEQ ID NO: 42 - DNA, 1443 bp, Hordeum vulgare
  misc_feature 1..1443: note = ORF of protein Cpk3
  source 1..1443: mol_type = unassigned DNA
caacagcccccaccccagcccgccctccccccagacgtcggccgggtgctgggccggccgatggaggacgtgcgcgccacctacaccttcggccgcgagctgggcaggggccagttcggggtcacctacctcgccacccacaagtcaaccggcgcgcgctacgcctgcaagtccatcgcggcccggaagctggcccgcgccgacgacgtggaggacgcgcggcgggaggtgcagatcatgcaccacctcacgggccaccgcagcatcgtcgagctgcgcggcgcctacgaggaccgccactccgtcaacctcgtcatggagctctgcgagggcggggagctcttcgaccgcatcatcgcccgcggccactactccgagcgcgccgccgccacgctctgccgcgaggtcgtcagcgttgtccacagctgccactccatgggggtcatgcaccgcgacctcaagcccgagaacttcttgttcttgaataagcgggaggactccccgctcaaggccaccgatttcgggctctccgtcttcttcaagcccggcgagcagttcagggatcttgttgggagtgcctattacgtggctccggaggtgctgaagcgccgatatggagccgaggccgacatttggagtgctggggtgatcctctacatcctcctatccggcgttcctccattctgggcagagaacgaggaaggtatatttgatgctgttctgaaaggtcacattgatttctcgtccgatccatggccatcgatttcaaatggggctaaagacttggtcaagaggatgctacggcaggaccccaaggagcggctcacggctgctgaaattttgaaccatccatggattagagaggatggcgaggccccagacaaaccacttgatattacagtgatcagtagaatgaagcagttcagggcgatgaacaagcttaagaaggttgccttgaagattgttgcagagagcttgtcagaggaagagatcgtgggcttaagagaaatgttcaagtccctggatactgataacagcgggacaattaccctggatgaactaagggctggcttaccaaagcttgggaccaaaattactgaatcagaaataagacagttgatggaggcggctgatgttgatggaaatgggaccattgattatgttgaattcatatcggcaacaatgcacatgaatagactagagaaggaagaccacatatttaaagcattcgaatactttgacaaggaccatagcgggtacataacagttgatgagctggaggaagcgctgaagaagtatgatatgggtgatgaggcaacaatcaaagatatcattgctgaagtggatacagatcatgatgggaaaattaactaccaggagtttgttgcgatgatgaagaataacagcccagagattgttccaaatcgacggcgcctattttaa

SEQ ID NO: 43 - DNA, 1785 bp, Hordeum vulgare
  misc_feature 1..1785: note = ORF of protein Cpk3
  source 1..1785: mol_type = unassigned DNA
cccaccccaacaccgcagcgcagatcacgcggtctccacggtcgtctctcccccaaaactcccacgctagcaccgctgctgcttcaaaccccgagcgagccccctatcgcctcgatctcccttcgtcaaacccattcgcacacaatgggcaaccgcacctcgcgccaccgccgcgccgccgccgaccagccggccgcggcccctccgccgacggcccagcccaaaccccagccgcagccgaagccgaagccacagacggcgccggccccggccccgacgccggaagcgggacaggtagccatgggccgcgttctgggacgtccgatggaggacgtgcgcgcgacctacaccttcggccgcgagctcggccgggggcagttcggggtcacctacctcgtcacgcacaaggctacgggccagcgcttcgcctgcaagtccatcgccacgcggaagctcgtccaccgcgatgacatcgaggatgtgcagcgcgaggtgcagatcatgcaccacctcacgggtcaccgcaacatcgtcgagctccgcggcgcctacgaggaccgccactcggtcaacctcatcatggagctgtgtgagggaggggagctattcgaccgcatcatcgcgcgggggcactactccgagcgagccgccgccttgctctgccgcgagatggtctccgttgtgcatagctgccactccatgggggttttccaccgggatctcaagcccgagaactttttgtttctgaataacaaggaggactcgccgctcaaggccactgacttcggcctctccgtcttcttcaaacacggggagcagtttaaggatcttgttggaagtgcgtattatgttgctcctgaggttctgaagcggcactatggagcagaagctgacatatggagtgctgggattattctgtacatccttctgtctggtgttcctcctttttgggcaggtgatgtgtgccaattctcatccgagttagattttctgctttttatttatccattaacacacatgttctattttccagacaatgaggatggcatatttgaggctgtcttgcttggtcacatagatttctcatctgacccttggccttcaatatctaatggtgcaaaagatttggtgaagaagatgttgcggcaagaccccaaagagcgcttgactgctgcggagattttgaatcacccatggatcagggaagatggagaggctccagataagccacttgacattactgttatcagtagaatgaagcagttcagggcgatgaacaagcttaagaaagttgcattgaagatcgttgcagagaacttgtctgaggaagagattacaggcttgaaagaaatgttcagatccctggacactgataacagtgggacaattactcttgaagagctaagatctggtttaccaaaacttggcaccaaaatttctgaatcagaaattacacagttaatggaggcggctgatgttgatggaaatgggaccattgattattctgagttcgtatcagcgacaatgcacatgaatagattggagaaggaagaccacatacttaaagcatttgaatattttgataaggaccacagcggatacataacagtagatgagctggaagaagctctgaagaagtatgacatgggggatgataaaacaattaaagatatcattgctgaagtagatacagatcatgatggaagaatcaactaccaggagtttgttgccatgatgagaaacaacagccctgagattgttccaaatcggcggcgcatgttctaa

SEQ ID NO: 44 - DNA, 1554 bp, Solanum tuberosum
  misc_feature 1..1554: note = ORF of protein Cpk3
  source 1..1554: mol_type = unassigned DNA
atgggcaactgtaacagcttatcttccgaccaagccgccaccacttcctccgccgccggcggtggcggcggcgccacttcagcgccgtcgaccaccggtatcagaatcatcccttcctcacaaacaccacctccacgaccattctccggcatcggccgtgttctcggccggccaatggaggatgtccggtccatctacattttcggcggcgaactcggccgcggtcaattcggagtcacctacttagttactaacaaaaaaacgcgccagcagttcgcgtgcaagtcaatcgctacacgaaaactcatcaacaaggacgatgtcgacgacgtccgccgtgaagttcagatcatgcatcaccttaccggtcaccggaacatcgttgagcttaaaggaacttttgaggataaacatcacgttcatctcgtcatggaactttgtgccggcggcgaactttttgaccggataattgctaaaggacactattccgagcgagctgctgctggagtttgccggcagatggtgactgttgttcacaattgtcattctatgggagttatgcatagggatttaaaacctgaaaatttcctgtttttgagctccgatgaaacttcaccattgaaggctactgattttgggctatcagtctttttcaaaccaggtgatgtgttcaaagacctggttggtagtgcatattatgtcgctcctgaagttctgcgccgagaatatggacctgaagctgatatttggagtgctggagttattctatacattctactcagcggtgttccaccattttatggagagaatgatcagagtatctttgatgctgttcttcgaggtcatcttgatttttcatctgatccttggccttcagtatcgagtagcgctaaagatcttgttaagaagatgctacgatctgatcctagagaaaggatttctgcagctgaagtcctaaatcatccatggatgagagaagatggtgatgcatctgacaaacctcttgacattgctgtcttatctagaatgaaacaattccgtgctatgaacaagttgaagaaagtagcactgaaggtaattgctgaaaatctttctgaagaagaaatcattggcttgaaggaaatgttcaaatctatagacacagatgacagtgggacaattacatttgaagaattgaaagctggtcttacaaaaatgggtacaaagctttctgagtcagaagtcaggcagttgatggaagcggctgatgttgatggaaatggaacaattgattaccttgagttcataacagctacgatgcacctgaatcgcatggaaagagaagatcacttgtacaaagcctttgagtattttgacaaggataagagcgggtatattacgatggaagaattggagcatgcgctgaaggaatacaacattactgatgaaaaaacaataaaagagatcattgctgaagttgacacagacaatgatggaagaatcaattacgatgagttcgctaccatgatgaggaaaggtaatccagattttgtcaacaataggcgccgcagatga

SEQ ID NO: 45 - DNA, 1578 bp, Amaranthus palmeri
  misc_feature 1..1578: note = ORF of protein Cpk3
  source 1..1578: mol_type = unassigned DNA
atggggaactgtaatggcttaccttccactctgtctcactcccgtctccgctccattgacgctaccaacaacgatactctctcctccacgttatcgccgcctccgccattacagcaacctcaacctcaacctccacaacctgcatcaccacaacctccaccgtcaaactccaatctcggccgtgttctaggtcgccctatcgaggatgttcgcgcaacatacactttcggccgcgaactaggtcgcggacaattcggagtaacttatttagttactcataaacaatctaagctacaatttgcttgtaaaaagattgctactcgtaatctgcttaatcaagatgatattgatgatgttcgtcgtgaagttcaaattatgcatcatcttactggtcatagaaacattgttgaacttaaaggtgcgtttgaagatcggcattatgttaatttggttatggaattgtgtgctggtggagagctttttgataggattattgctaaaggacactattctgaacgtgaagcggctaaattgtttaggcagattgttaccgttgtgtataattgtcattctatgggagttatgcatagagatcttaagccggagaatttcctgtttttgagctcagatgaagagtcgccattgaaagctactgattttggtctatctgtcttctttaagcctggagaagtcttcagagaccttgtcggaagcgcatattatgtagctcctgaggttttacgtcgtagttatggggctgaagctgatgtttggagtgctggagtgatattatacatactacttagtggagttccaccattttggggagagaatgagcagggaatttttgattctattctgcggggcaatcttgatttcaattcagatccatggctctcaatatctagcagtgccaaagacctggtgacaaagatgttgcggcaggatcctaaggaaagactttctgctcgtgatgctttaaatcatccttggatgagagaggatggggaagctcctgataagcctattgatattgctgttttgactagaatgaagcagtttagggcaatgaacaaactgaaaaaagtagcactaagggtaattgccgagaacctttctgaagaaaaaataattggtttaaaagaaatgtttaaatctatagatactgataatagtggaactatcacctatgaagaattaaagagcggtcttccaaagctgggaactaaactttctgagaaagaagtcaagcagttaatggaggcggctgacgtcgatggtaatggaaccatagattacatggaattcatcacagctacaatgcatatgaataggatggaggaagaggaccatatatacaaagcctttgaatattttgatgtggataaaagcgggtacattactatggaggaattggcgcaagccttgaagaggtataatatgggtgatgcaaaaacaatacgggagataattgcagaagttgatacagacaatgatggtaaaatcaattatgatgaatttgtagcgatgatgaagaaaggcaaccctgatatggttaccaacagacggaagagatga

SEQ ID NO: 46 - DNA, 1551 bp, Medicago truncatula
  misc_feature 1..1551: note = ORF of protein Cpk3
  source 1..1551: mol_type = unassigned DNA
atggggggcaactgcagcaccggcgccggcgccggaatcaacaccacaaccaccaccccccattccgatcctccacccaaaaacgccccaacaatcctcccacaaaacttcactccctcaccccctcaaccacccttacccaccaccaccctcggccgcatcctgaaccgtccaatggaagacgttcgctccatcttcatcttcggccgtgaactcggccgaggccaattcggcgttacatatttagtaacccacaaagttaccaaagaacaattcgcttgtaaatcaatcgctacacgtaaactcgtcaaccgtgacgatatcgatgatgtccgccgcgaggttcaaatcatgcatcatctgaccggtcatcgtaacattgttgaactcaaaggtgcttatgaggataaacactctgttaaccttgttatggagctttgtgctggtggtgaactttttgatcggattattctcaaaggacattactctgaacgtgctgctgctaatctttgtcgtcagattgttacggttgttcataattgtcatactatgggagttatgcatagggacttgaaacctgagaatttcttgtttcttggtaaagaagaaaattcgcctctcaaggccacggattttggtctctctgtgtttttcaagccaggagacgtgtttaaagatcttgttgggagtgcatattatgttgctcctgaagtgctgcgcaaaagttatgggcctgaaactgatatttggagcgccggaattatattatatattctgctttctggtgtcccccccttctgggcagaaaatgaacagggtatctttgatgctattcttcgtggacaaattgattttgcgtcggatccttggccttctatatcaagcagtgccaaagatctggtcaagaagatgctacgaaatgacgctaaagaacggatatcagcagttgaagtactcaatcatccttggatgagagtagatggagatgcatctgacaagcctcttgatattgctgttttgagcagaatgaaacagttccgagcaatgaacaagctaaagaaagtagctttaaaggttatcgccgaaaatctttctgaagaagaaattattggcttgaaggaaatgttcaaatccatggatacagacaatagtggaacaatcacttttgaagagttgaaagctggcctccctaaactcggtagtaaaatgtcagagtctgaagtaaggcagttgatggaagcggctgacgtggacggtaatggaaccatcgattatattgagtttataaccgctaccatgcatatgaatagaatggaaagagaggatcacctatataaagcctttgagtattttgaccaagataagagcgggtacatcacaaaggaagagttggaatctgcccttaagaagtataatatgggcgacgagaacacaataaaggagatcattgccgaagttgatacagacaacgatgcaagaattaactatgatgagtttgtagctatgatgaggaaaggcaacccagatataacccacaggcgtcgcaaataa

SEQ ID NO: 47 - DNA, 1554 bp, Medicago truncatula
  misc_feature 1..1554: note = ORF of protein Cpk3
  source 1..1554: mol_type = unassigned DNA
atgggcaaccgcatcagctccaaacacaaaccctcttccgattcccaaacccgttccaaatcagaaccctcacattccactcccacttccttccatcctcctaatcgaacccccaaacaatccactcacacccccaccccttcctcctcccccctcggccgcgtcttaggcaaacccatggaagacgtccgatcaacctacaccttcggccgtgaactcggtcgcggccaattcggtgtaacctacttagttacacacaaactcaccaaagaacaattcgcctgcaaatctatagccactcggaaactaatccgccgcgatgatcttgacgatattcgccgtgaagttcagatcatgcatcatctcaccggtcaccggaacattgttgagcttaaaggtgcttatgaggatcgtcattcggttaatcttatcatggagctttgtgctggtggtgagctttttgataggattatatccaaaggtcattactctgaacgtgctgcggctgagctgtgtcgccagattgtcactgttgttcataattgtcatactatgggtgttatgcatagggatttgaagcctgagaatttcttgtttcttagtaagcatgagaattcgccgcttaaagctacagattttggtctctccgtgtttttcaagcccgaagatgtattcaaagatcttgttgggagtgcatattatgttgctcctgaggtgttgcgtagaagctatggacctgaagctgatatttggagtgctgggattattttgtacattctcctctctggtgttcctcccttctgggcagaaaatgaacaaggtatcttcgatgctattcttcgtggacatcttgatttcgcatctgatccttggcctaaaatatcgagtattgctaaagatctgatcaaaaagatgttgcgagctgaccctaaagaacgaatttcagctgttgaagtccttgaccattcttggatgaaagaagatggagcatccgataagcctcttgatattgctgtcttaaccagaatgaaacaattcagggcaatgaacaagctaaagaaagtagctctaaaggttattgctgaaaatctttctgaagaagaaattataggcttgaaggaaatgtttaaatccatggatacagataacagtggaacaatcacttttgaagagttgaaagctggtctccccaaactaggtactaagatttcggaatctgaagtaaggcagttgatggaagcggctgatgttgacggaaatggaaccattgattatattgaatttataacagctaccatgcacttgaatagaatggaaagagaggaccacctatacaaagcttttgaatattttgacaacgacaaaagcgggtatattacaaaagaagagttggaatctgcccttacaaagtacaacatgggtgatgagaaaacaataaaggagataattgacgaagttgattcagacaatgatggaagaattaactatgaggagtttgtagccatgatgaggaaaggcaatccagacttgataaccaacaaacgtcgcaaataa

SEQ ID NO: 48 - DNA, 1008 bp, Triticum aestivum
  misc_feature 1..1008: note = ORF of protein Cpk3
  source 1..1008: mol_type = unassigned DNA
atgggggttttccaccgggatctcaagcccgagaactttttgtttctcaacaacaaggaggactcgccgctcaaggccactgactttggcctctccgtcttcttcaaacacggggagcagtttaaggatctcgttggaagtgcgtattatgttgctcctgaggttctgaagcggcactatggagcagaagctgacatatggagtgcggggattattctgtacatccttctgtctggtgttcctcctttttgggcagacaatgaggatggcatatttgaggctgtcttgcttggtcacatagatttctcatctgacccttggccttcaatatctaatggtgcaaaagatttggtgaagaagatgttgcggcaagaccccaaagagcgcttgactgctgcagaaattttgaatcacccatggattagggaagatggagaggctccagataagccacttgacattactgttatcagtagaatgaagcaattccgggcgatgaacaagcttaagaaagttgcattgaagatcgttgcagagaacttgtctgaggaagagataacaggcttgaaagaaatgttcagatccctggacactgataacagtgggacaattactcttgaagagctaagatctggtttaccaaaacttggcaccaaaatttctgaatcagaaattacacagttaatggaggcggctgatgttgatggaaatgggaccattgattattctgagttcgtatcagcgacaatgcacatgaatagattggagaaggaagaccacatacttaaagcatttgaatattttgataaggaccacagcggatacataacagtagatgagctggaagaagctctgaagaagtatgacatgggggatgataaaacaattaaagatattatcgctgaagtagatacagatcatgatggaagaatcaactaccaggagttcgttgccatgatgagaaacaacagccctgagattgttccaaatcggcggcgcatgttctaa

SEQ ID NO: 49 - DNA, 903 bp, Triticum aestivum
  misc_feature 1..903: note = ORF of protein Cpk3
  source 1..903: mol_type = unassigned DNA
aaatggcgggagcagtttaaggatctcgttggaagtgcgtattatgttgctcctgaggttctgaagcggcactatggagcagaagctgacatatggagtgcggggattattctgtacatccttctgtctggtgttcctcctttttgggcagacaatgaggatggcatatttgaggctgtcttgcttggtcacatagatttctcatctgacccctggccttcaatatctaatggtgcaaaagatttggtgaagaagatgttgcggcaagaccccaaagagcgcttgactgctgcagaaattttgaatcacccatggattagggaagatggagaggctccagataagccacttgacattactgttatcagtagaatgaagcagttccgggcgatgaacaagcttaagaaagttgcattgaagatcgttgcagagaacttgtctgaggaagagattacaggcttgaaagaaatgttcagatccctggacactgataacagtgggacaattactcttgaagagctaagatctggtttaccaaaacttggcaccaaaatttctgaatcagaaattacacagttaatggaggcggctgatgttgatggaaatgggaccattgattattctgagttcgtatcagcgacaatgcacatgaatagattggagaaggaagaccacatacttaaagcatttgaatattttgataaggaccacagcggatacataacagtagatgagctggaagaagctctgaagaagtatgacatgggggatgataaaacaattaaagatattatcgctgaagttgatacagatcatgatggaagaatcaactaccaggagttcgttgccatgatgagaaacaacagcccggagattgttccaaatcggcggcgcatgttctaa

SEQ ID NO: 50 - DNA, 897 bp, Triticum aestivum
  misc_feature 1..897: note = ORF of protein Cpk3
  source 1..897: mol_type = unassigned DNA
ggcgagcagttcagggatcttgttgggagtgcatactatgtggctccagaggtgctgaagcggcgatatggagccgaggccgacatttggagtgctggagtgatcctctacattctgctctccggcgttcctcctttctgggcagagaatgaagaaggtatatttgatgctgttctgaaaggtcacattgatttctcgtctgatccctggccatcgatttcaaatggggctaaagacttggtcaagaggatgctacggcaggaccccaaggagcggctcacggctgcggaaatattgaatcatccatggatcagagaggatggagaggccccagacaaaccacttgatattacagtgatcagtagaatgaagcagttcagggcgatgaacaagcttaagaaggttgccttgaagattgttgcagagagcttgtcagaggaagagatcgtgggcttaagagaaatgttcaagtccctggatactgataacagtgggacaattactcttgatgaactaagggctggcttaccaaagcttgggaccaaaattactgaatcagaaataagacagttaatggaggcggctgatgttgatggaaatgggaccattgattatgttgaattcatatcggcaacaatgcacatgaatagactagagaaggaagaccacatatttaaagcatttgaatactttgacaaggaccacagcgggtacataacagttgatgagttggaggaagctctgaagaagtatgatatgggtgatgaggcaacaattaaagatatcattgctgaagtggatacagatcatgatgggaaaattaactaccaggagtttgttgcgatgatgaagaacaacagtccagagattgttccaaatcgacgccgcctattttaa

SEQ ID NO: 51 - DNA, 1407 bp, Triticum aestivum
  misc_feature 1..1407: note = ORF of protein Cpk3
  source 1..1407: mol_type = unassigned DNA
atgggccgcgttctgggacgcccgatggaggacgtgcgcgcgacctacaccttcggccgcgagctcggccgggggcagttcggggtcacctacctcgtcacgcacaaggctacgggccagcgcttcgcctgcaagtccatcgccacgcggaagctcgtccaccgcgatgacatcgaggatgtgcagcgggaggtgcagatcatgcaccacctcacgggtcaccgcaacatcgtcgagctccgcggcgcctacgaggaccgccactcggtcaacctcatcatggagctatgtgagggcggggagctattcgaccgcatcatcgcgcgggggcactactccgagcgagccgccgccctgctctgccgcgagatggtctccgtcgtgcacagctgccactccatgggggttttccaccgggatctcaagcccgaaaactttttgtttctcaacaacaaggaggactcgccgctcaaggccactgacttcggtctctccgtcttcttcaaacacggggagcagtttaaggatctcgttggaagtgcgtattatgttgctcctgaggtactgaagcggcactatggagcagaagctgacatatggagtgcggggattattctgtacatccttctgtctggtgttcctcctttttgggcagacaatgaggatggcatatttgaggctgtcttgcttggtcacatagatttctcatctgacccctggccttcaatatctaatggtgcaaaagatttggtgaagaagatgttgcggcaagaccccaaagagcgcttgactgctgcagaaattttgaatcacccatggattagggaagatggagaggctccagataagccacttgacattactgttatcagtagaatgaagcagttccgggcgatgaacaagcttaagaaagttgcattgaagatcgttgcagagaacttgtctgaggaagagattacaggcttgaaagaaatgttcagatccctggacactgataacagtgggacaattactcttgaagagctaagatctggtttaccaaaacttggcaccaaaatttctgaatcagaaattacacagttaatggaggcggctgatgttgatggaaatgggaccattgattattctgagttcgtatcagcgacaatgcacatgaatagattggagaaggaagaccacatacttaaagcatttgaatattttgataaggaccacagcggatacataacagtagatgagctggaagaagctctgaagaagtatgacatgggggatgataaaacaattaaagatattatcgctgaagttgatacagatcatgatggaagaatcaactaccaggagttcgttgccatgatgagaaacaacagcccggagattgttccaaatcggcggcgcatgttctaa

SEQ ID NO: 52 - DNA, 1524 bp, Lolium perenne
  misc_feature 1..1524: note = ORF of protein Cpk3
  source 1..1524: mol_type = unassigned DNA
atgggcggccgcgcctcccgcccgcgcgccccaccgcccgacgagcaccaccagtcctcccaccccaagcagcccaactggccgcgccccaagaaccggcgccaaccccctcccctccctgcccctcccccacagccgcagccccccgccccccagccccagcccgcgctccccccagacgtcggccgcgtcctcggccgcccaatggaggacgtgcgcgcgacctacaccttcgggcgcgagctgggccgcggccagttcggcgtcacctacctggccacccacaaggcctccggcgccaggcgcgcctgcaagtccatcgccgcccgcaagctcgcgcacgcctccgacgccgaggacgtgcgccgcgaggtgcagatcatgcaccacctcaccggccaccgcagcatcgtcgagctgcacggcgccttcgaggaccgccactccgtcaacctcgtcatggagctatgcgagggcggcgagctcttcgaccgcatcatcgcccgcggacactactccgagcgcgccgccgccgcgctctgcagggaggtcgtctccgtcgtccacagctgccactccatgggggtcatgcatagggatctaaagcccgagaatttcttgtttctcaataagagggaggactcgccgctcaaggccaccgatttcggactatcagtcttcttcaagccaggtgagcagttcaaggatcttgttgggagcgcgtattatgtcgctccggaggtgctcaagcggcgatatggtgccgaggcagacatatggagtgccggagttatcctctacattctcctctccggcgtccctcctttctgggcagagaacgaggatggtatattcgacgccgttcttaaaggccatatagatttctcgtcggaaccctggccatcgatttcaaatggtgcaaaagacttggtcaagaggatgctaaagcaagaccctagggagcggcttactgctgcggaaattttgaaccatccatggattagagaggatggagaggccccagacaaaccacttgatattacagtgatcagtagaatgaagcagttcagggcgatgaacaagcttaagaaggttgccttgaagatcgttgcagagagtttgtcagaggaagagatcatgggcttaagagaaatgttcaaatcgctggatactgataacagtggtacaattactcttgaagaactaagggctggcttaccaaagcttggtaccaaaatcaccgaaccagaattgagacagttgatggaagcggctgatgttgatggaaatgggaccattgattatggcgaattcatatcagcaaccatgcacatgaatagactagagaaggaagatcacatatttaaagcatttgagtattttgacaaggaccatagcgggtacataacagttgatgagttggaagaagctctcaagaagtatgatatgggtgacgaggcaacaatcaaagatatcattgctgaagtggatacagatcatgtgagtaactccagtaaactgtaa

SEQ ID NO: 53 - DNA, 1587 bp, Lolium perenne
  misc_feature 1..1587: note = ORF of protein Cpk3
  source 1..1587: mol_type = unassigned DNA
atgggcggccgcgcctcccgcccgcgcgccccaccgcccgacgagcaccaccagtcctcccaccccaagcagcccaactggccgcgccccaagaaccggcgccaaccccctcccctccctgcccctcccccacagccgcagccccccgccccccagccccagcccgcgctccccccagacgtcggccgcgtcctcggccgcccaatggaggacgtgcgcgcgacctacaccttcgggcgcgagctgggccgcggccagttcggcgtcacctacctggccacccacaaggcctccggcgccaggcgcgcctgcaagtccatcgccgcccgcaagctcgcgcacgcctccgacgccgaggacgtgcgccgcgaggtgcagatcatgcaccacctcaccggccaccgcagcatcgtcgagctgcacggcgccttcgaggaccgccactccgtcaacctcgtcatggagctatgcgagggcggcgagctcttcgaccgcatcatcgcccgcggacactactccgagcgcgccgccgccgcgctctgcagggaggtcgtctccgtcgtccacagctgccactccatgggggtcatgcatagggatctaaagcccgagaatttcttgtttctcaataagagggaggactcgccgctcaaggccaccgatttcggactatcagtcttcttcaagccaggtgagcagttcaaggatcttgttgggagcgcgtattatgtcgctccggaggtgctcaagcggcgatatggtgccgaggcagacatatggagtgccggagttatcctctacattctcctctccggcgtccctcctttctgggcagagaacgaggatggtatattcgacgccgttcttaaaggccatatagatttctcgtcggaaccctggccatcgatttcaaatggtgcaaaagacttggtcaagaggatgctaaagcaagaccctagggagcggcttactgctgcggaaattttgaaccatccatggattagagaggatggagaggccccagacaaaccacttgatattacagtgatcagtagaatgaagcagttcagggcgatgaacaagcttaagaaggttgccttgaagatcgttgcagagagtttgtcagaggaagagatcatgggcttaagagaaatgttcaaatcgctggatactgataacagtggtacaattactcttgaagaactaagggctggcttaccaaagcttggtaccaaaatcaccgaaccagaattgagacagttgatggaagcggctgatgttgatggaaatgggaccattgattatggcgaattcatatcagcaaccatgcacatgaatagactagagaaggaagatcacatatttaaagcatttgagtattttgacaaggaccatagcgggtacataacagttgatgagttggaagaagctctcaagaagtatgatatgggtgacgaggcaacaatcaaagatatcattgctgaagtggatacagatcatgatgggaagattaactatgaggagtttgttgcaatgatgaagaataacggcccagagattgttccaaacagacggcgactattttaa

SEQ ID NO: 54 - DNA, 1581 bp, Lolium perenne
  misc_feature 1..1581: note = ORF of protein Cpk3
  source 1..1581: mol_type = unassigned DNA
atgggcaaccgcacctcgcgccaccgccgcgccgccgccgagcagccggcgacgcccccgccgcccgcgcagcccaagccccagcccaagcaggagacgccgccgcaccgcccgcagccgacgacggcgccgcccccctgcccagccccggcgccggaggcggcggggcaggcgatgggccgcgtcctggggcgcccgatggaggacgtgcgcgcgacctacaccttcgggcgcgagctgggccggggccagttcggggtcacctacctcgtcacgcacaaggccacggggcagcgcttcgcctgcaagtccatcgccacccggaagctcgtccaccgcgacgacatcgaggacgtgcagcgggaggtgcagatcatgcaccacctcacgggccaccgcaacatcgtcgagctccgcggcgccttcgaggaccgccactcggtcaacctcatcatggagctctgcgagggcggggagctcttcgaccgcatcatcgccaggggacactactccgagcgggccgccgcggggctatgccgcgagatggtctccgtcgtgcacagctgccactccatgggggtattccaccgggatctcaagcccgagaactttctattcctcaataacaaggaggactcgccgctcaaggcaactgacttcggcctatccgtattcttcaagcatggggagcagtttaaggatctcgttggaagtgcatattatgttgctcccgaggttttgaaacgccactatggagcagaagctgacatatggagtgctgggattattctctacatccttctgtccggtgttcctcctttttgggcagacactgaggatggcatatttgaggctgtcttgcttggcaacatagatttctcatctgacccttggccttcagtatccaatggtgcaaaagatttggtgaagaagatgctgcggcaagaccccaaagagcgcttgactgctgcggagattttgaatcacccatggattagagaagatggagaggccccagacaagccacttgacatcacagttatcagtagaatgaagcagttcagggcaatgaacaaactcaaaaaagttgcattgaagatcgttgcagagaacttgtctgcagaagaaattactggcttgaaagaaatgttcagatccttggatactgataacagtggtacaattacactggaagagctaagatctggtttaccaaaacttggcaccaaaatttctgagtcagaaattgcacagttaatggaggcggctgacgttgatggaaatgggaccattgattattctgagttcgtgtcagccacaatgcacatgaatagattggagaaggaagaccacatactgaaagcatttgaatattttgataaggaccacagcggatacataacagtagatgagttggaagaagctctgaagaagtatgacatgggagatgataaaacaatcaaagaaatcatcgctgaagtagatacagataatgatggaagaatcaactaccaggagtttgttgccatgatgagaaacaacagccctgagatcgttccgaatcggcggcgcatgttctaa

SEQ ID NO: 55 - DNA, 1581 bp, Lolium perenne; identical to SEQ ID NO: 54
  misc_feature 1..1581: note = ORF of protein Cpk3
  source 1..1581: mol_type = unassigned DNA

SEQ ID NO: 56 - DNA, 2031 bp, Arabidopsis thaliana
  misc_feature 1..2031: note = ORF of protein Crk34
  source 1..2031: mol_type = unassigned DNA
atgaagctaaagatttcgtttttaccaaccttcttgatttttctcataagcttagattctgttactgctcaagaaatctgtttcagtggattctttaaacccaatagtacatacgatttaaaccgtcgtcaaatcctctctactctttcttctaatgtcacaagtcacaacggctttttcaactccaagtttggtcaagctcccaatagagtcttcatcaatggcatgtgcatccctgggactaaaccagagacttgttcagattgtattaaaggtgcatcagacaagatatcagagagttgtcctaacaaaacagatgcttatacatggcctgattgttgtatggtacgttattccaacgtttctttctctggttcactcgttatggaaccttctgaaactctctatcatacgggagatatcgaagatacaggcactaatttaacagtgtttgatagaatttgggaagagttaatgctccgtacaataactgcagcttcattgagtagtagtaatggatcatcatttggacaaaagtactttgcagctgaggttgcttcattgactactttccaaaccatgtatgcgatgatgcaatgtacgccggatgtttcttcgaaagattgtgagttttgtttgaagacaagtgttggtgattatgagtcatgttgccgtggtaaacaaggtggtgcggtgataagaccaagttgctttgtccggtgggatttgtatccttatgctggagcttttgaaaatgttacatttccaccgcctccaccgcagtctttacctcagccaccggtttctttgattcctcctccagttagcgatagggcaaatacaaccatcaaagatggaaaatcattttctacagggattattgtggctattgttgttcctatcattgttatcttggtttcacttgttgtactactcgtggtttgcagaagaaagaagtcatataaaacaactgaggttcaagctactgatgagatcacaacaacacactcactgcagtttagttttaagacaattgaagctgcaactgacaagttttcggacagtaacatgatcgggcgaggtggatttggtgaagtttacaggggaaagctttcaagtggacctgaagtagcagtgaagagactgtcgaaaacatcaggacaaggcgcggaagagttcaagaatgaggctgttcttgtgtcgaagcttcaacataagaatctggtcaggcttcttggattttgtttggaaggagaagaaaagatactcgtctatgagtttgtacccaacaaaagccttgactattttctctttgaccctgcaaaacaaggtgaactagattggacaagacggtacaatatcattggggggattgctcgaggaattctatatcttcatcaagattcacggcttacaatcatacaccgtgatctcaaagcgagtaacattctcttagatgctgatatgaaccccaaaattgcagattttggcatggcgaggatttttggagttgatcaaagtcaagccaacacaagaagaatcgccggaacatttggttatatgtctccagaatatgcaatgcgtggccatttctccatgaagtctgatgtctatagctttggagtattggtactagagattataagtggcaaaaagaatagcagtttctacaacattgatgattctggtagcaatttggtaacacacgcttggagactttggagaaatgggtcaccgttagaactggtggatccaaccataggagaaagttatcagagtagcgaggctactagatgcatccatatcgcactattatgtgttcaagaagatcctgcagatcgtccattgttaccagcgatcatcatgatgcttactagtagcacaaccactttacatgtgcctcgagcgcccggattttgcctaagcgggcgtgatctggaacaggatggtgtagagtataccgaatccacaagcaggtctattcctggttctatcaatgatgcatcgattacagagttctatcctcgttga

SEQ ID NO: 57 - DNA, 1575 bp, Arabidopsis thaliana
  misc_feature 1..1575: note = ORF of protein Cyp705a18
  source 1..1575: mol_type = unassigned DNA
atggcaacacttatcagcgttgactttcagcactgtttcatgtacataatcctcctatgcctcttctcacttctctgttactctctcttcttcaggaaaccaaagggcacacgagttggctgtgatctgcctccgagccctccgtctcttcctatcattggtcatcttcaccatctcctctcttctctaactcacaagtctttacaaaaactctcctccaagtatggacctttcctccatctccggatctttagtgttcccatcatccttgtctcatctgcttcagtggcatatgagatctttaaggcccacgatgtgaacgtctcatctcgtggtgctgctgcgatcgatgagtccctcttgtttggatcttctagcttcatttacgctccctatggagattactggaagttcatgaagaagatcttcgccactaagctgctccgaccacaggtgctggagaattcacgtggtgttcgtgctgaagagttaaagcggttttacaggagaattctcgataaggcgaggaagaatgagaacatcgagatcagtaaggaatcggcgatgctcatgaacaacatcttatgcaggatgagcatgggaaggagtttttcagaggaaaatggtgaggcagagagagtcaggggcctggtgggtgaatcatatgccttggtgaagaagattttcttcgcagctacattgcgcagactgcttgagaagcttcggatcccactattcaggaaggagataatgggtgtgtccgacagatttgatgagctgctagagaggattattgtggaacacaaagacaaacttgaaaaggagcatcaagttatggatatgatggatgtgttgttggcagcttatcgagacaaaaacgcagagtataagatcactaggaatcatatcaagtcgttttttgtggaccttttcgttgggggcactgatacctcggtccaaacaacacaatggactatggctgagatcattaataaccctaacattcttcagacactgagaaaagaaatcgattcagttgtagggaaatcaaggttgattcacgaaacggatataccaaaccttccttatttgcaagcagtggttaaggaaggactaagattgcatcctccagggcccctcttgataaggacttttcaagaaagatgtgagatgaaagggttctacataccggagaagacaacactcgttatcaacgcttatgctgtgatgagagatcccgactcttgggaagatcctgacgagtttaagccagagaggtttctaagttattcaagatcagggcaagaagacgagaaagagcaaacacttaagtacctttcttttggtggcggaaggagaggatgtcctggagtaaatctaggttatatatttgttggaactgcaataggaatgatggtgcagtgctttgactggaaaatcgaaggagataaggtcaacatggaagagacttatggaggaatgaacctaaccatggttaacccgcttaagtgcactccagttcctcgaacccaacctttttcttttacttctaatctcgagattcttagttcctga

SEQ ID NO: 58 - DNA, 1503 bp, Arabidopsis thaliana
  misc_feature 1..1503: note = ORF of protein Cyp71b26
  source 1..1503: mol_type = unassigned DNA
atggatagcatttggattctatcacttctcttcttcatcatctttcttcttctggccgccttcaaacgcaaaaatcatgggaaacatcgacgaataccatctcctcctggttttccaatcatcggaaacttacatcagctcggagaattacaacatcagtctctatggaagctctcaaagaagtatggccctgtgatgcttttgaagcttggaaaagtccccacactcatactttcttcatcagaaacagcaaaacaagctctaagagactatgatctccattgttgtagccgtccttccttagcaggaggaagagagctctcttacaacaatctagatatgtcttcatctccttataatgaatattggaaggaactaaggaagctctgcagtcaagaactctttagtgctaataaaattcaatcgattcaacctattaaggacgaggaggtcaagaaagttatcgattcaatcgcggaatcatcttctctaaagaatccggttaacttgagcaagacgtttcttgctttaactacaagtgtagtatgcaaggcagcatttggtgtgagttttgagggaagtgtgctcaacagtgatagattcaataagttagtccgagatactttcgagatgttgggaagcttctctgcctcagattttattccgtatgtcggatggatcatcgataagttcaatggtttacaagggtggagaaagaaaagctttcgagatcttgatgcgttctatgaacaaatttttgatctgcataaggaagagaaggaagtaggaagtgaagatttagtggatgtgctcttgaggttggagaaagaagaaattgttgttggaaatggcaagctcacaagaaatcatatcaaagcaattttgatgaacattcttttaggaggaatcgatacttctgcaataacaatgacatgggcaatggcagaacttgctaaaaaccctagagtgatgaagaaagttcaagcagaaatcagaaaccaaatcaagaacaaagaaagaatcagctttgatgacactgataagctcgagtacttgaaaatggtgatcaaagaaacatggaggttacatcctccaacacctcttctcctcccaagagatgtaatcactgaatttgagatcaatggctacacgattcctgccaaaacacggcttcatgtgaatgtttgggctatcgggcgtgatcctgatacatggaaagatccagaaatgtttctccccgaaaggtttaatgatagtaacattgatgcaaaaggacagaactttgagttgttgtcgtttgggagtggtaggagaatttgtcctggactgtacatgggaacaacaatggtggagtttggcctagctaatatgttgtatcattttgattggaagttaccagaaggcatggtagtcgaagatatcgatatggaagaagctcctggacttactgtgagcaaaaaaagtgagcttgtacttgttccagtgaagtatttagaccattga

SEQ ID NO: 59 - DNA, 1608 bp, Arabidopsis thaliana
  misc_feature 1..1608: note = ORF of protein Cyp78a8
  source 1..1608: mol_type = unassigned DNA
atgagaaccgaaatagaaagtttgtgggtatttgctcttgcatcaaaattcaatatttacatgcaacaacattttgcttcccttctcgtcgccattgctatcacttggtttaccataaccatcgtattttggtctactccgggtggaccggcttggggaaaatacttcttcactcgccggtttatttctctcgattacaaccgaaaatacaagaatctcattcccggtcctagagggtttccacttgtgggaagcatgagccttaggtcaagccacgtggctcatcagcgcatagcgtctgtggctgagatgagtaacgccaagcggctcatggcgtttagcctcggtgatactaaggtggtggtgacgtgtcatcctgccgtggcaaaggagatactaaacagttcggtttttgctgaccgaccggttgacgaaaccgcttacggtttgatgtttaaccgagccatgggatttgctcccaatggtacttattggcgtacgctgcgtcggttaggctcgaaccatctttttaacccgaagcaaatcaaacaatcggaggatcagagacgggtgatagcgactcagatggtgaatgcgtttgcacgtaaccctaaatccgcgtgtgcagtgcgtgatttgctcaaaacagcgtcgttgtgtaacatgatgggtttggttttcgggagagagtatgaattggagtcaaataacaacttggaatctgaatgcttaaagggtttggttgaagaagggtacgatcttctaggtacgttaaattggaccgaccatcttccttggttagccggtttagatttccaacaaatccggtttaggtgctcgcagctcgtaccgaaagtaaatctgttattgagccgtatcatacatgaacaacgtgctgccacgggtaactttcttgacatgttactttctcttcaaggttcagaaaaattatcagaatccgacatggttgctgttctttgggaaatgatatttaggggaacggacactgttgcggttttggtcgagtgggtgctagcgaggattgtgatgcatcccaaagttcaattaacggtccacgatgagcttgaccgagtcgttggcagatcaagaaccgtggatgagtcagaccttccatcactcacgtatctaacggctatgatcaaagaagtgttgaggctgcatccaccaggtccactgctttcttgggcacgactgtctataacagacacttccgtagatggatatcacgtgccggctgggaccaccgcgatggtcaacatgtgggctatagcacgtgacccacacgtgtgggaggatcctttagagtttaagcctgagaggttcgtggctaaagagggtgaagctgagttctctgttttcgggtcggatctgaggttggcaccgttcgggtcgggtaagagggtttgccctggaaagaatttgggacttacaacggtgtcgttttgggttgcaacgctcttgcatgagtttgagtggcttcctagcgtcgaagctaaccctccagatctctcggaggttttgaggctctcgtgtgagatggcttgtccactcatcgttaacgtaagctcaaggcgtaagataatgtaa

SEQ ID NO: 60 - DNA, 1743 bp, Arabidopsis thaliana
  misc_feature 1..1743: note = ORF of protein Cyp97b3
  source 1..1743: mol_type = unassigned DNA
atggtagcagccatggcttttcctgccgctgctacttatcccacccatttccaaggcggcgctcttcatctgggtaggaccgatcattgcctcttcggtttctaccctcaaaccatttcctctgtgaattctcggagagcttctgtttccatcaagtgccaatctacggagccaaagacgaatggtaacatattggacaatgcgagcaaccttttgacaaattttttaagtggtggaagtttggggtcaatgcctactgctgaaggctctgtctctgatttgtttggaaagcctctctttttatctctttacgactggttcttggagcatggaggaatttataaacttgcgtttggtccaaaagcctttgttgtcatctcagatcccattattgcaaggcatgtcctccgggaaaatgctttttcttatgacaagggagttcttgctgagatcttagagccgattatgggaaaagggttaataccggctgatctagatacgtggaagttaagaagaagagctatcactcccgcattccataaattgtatctagaggccatggtcaaagtatttagtgactgttcggagaaaatgatattgaaatctgagaaactcataagggagaaagaaacttcaagcggggaggacaccattgagttggatctggaagcagaattctcgagtctggctcttgatattataggtcttagcgtgttcaactacgattttggctctgtcacaaaagagtcccctgtgatcaaggcagtttatggaactcttttcgaggcagagcatcggtctactttctacttcccttattggaactttcctccagctagatggatagttccgaggcaacgaaagttccaaagcgatctgaagattataaacgattgccttgatggcctcattcaaaatgctaaagagacaagacaggaaacagatgttgagaagctccaggaaagggactacactaatctcaaggatgcaagtcttttgcggttcttagtcgatatgcgcggtgttgacattgatgaccggcagctgagggatgacttgatgactatgctaattgctggtcatgagacaacagcagcagtacttacttgggctgttttccttctgtcacaaaatcctgaaaaaattaggaaagctcaagctgagattgatgctgtgcttggtcaaggtccacccacttatgaatcaatgaaaaagctcgagtacatacgactgatcgttgtagaagtccttcgtctctttcctcagccacctttgctcatcagacgcactctcaaaccagaaacattacccggaggacacaaaggggaaaaagaaggtcataaagttccaaaagggactgatatcttcatttctgtgtataatctccatagatctccatacttttgggataatccccacgattttgagcctgagaggtttttaagaacaaaggagagcaatggaattgaaggatgggctggctttgatccatctcgtagccccggggcactatatccgaatgagataatagcagactttgcattcttaccatttggtggaggaccaagaaaatgcattggagaccagtttgcactaatggaatcgaccgtcgcactagctatgttgtttcagaaattcgatgtggagctgcgtggaacgccagaatctgttgaactcgtgagcggcgcaacgattcatgccaaaaatgggatgtggtgcaaactaaagagaagatcaaagtga

SEQ ID NO: 61 - DNA, 5730 bp, Arabidopsis thaliana
  misc_feature 1..5730: note = ORF of protein Dcl1
  source 1..5730: mol_type = unassigned DNA
atggtaatggaggatgagcctagagaagccacaataaagccttcttattggctagatgcttgcgaggacatctcttgtgatcttatcgatgatctcgtgtctgaatttgatccttcctctgttgctgtcaatgaatccactgatgaaaacggcgtcatcaatgattttttcggtgggattgatcacattttagatagtatcaagaacggtggaggcttaccaaacaatggcgtttctgataccaattctcaaatcaacgaggttactgtaactcctcaggttattgctaaggagacagtgaaggagaatgggttgcaaaagaatggcggtaagagagacgaattctcgaaagaggaaggagacaaggataggaagagagctagggtttgtagttatcagagtgaaaggagtaacctttcaggtagagggcatgttaataattctagggagggagataggtttatgaataggaaacgtactcgtaattgggacgaggcgggtaacaataagaagaaaagggaatgtaacaattacagaagagatggtagagatagagaagttaggggttattgggagagggataaagttggttccaatgagttggtttataggtcagggacttgggaagctgatcatgaaagagatgttaagaaagtgagtggtggaaaccgcgaatgcgatgtcaaggcagaggagaacaagagtaagcctgaagaacgtaaagagaaggttgtggaagagcaagcaaggcgataccagttggatgttcttgaacaagctaaagcgaaaaacacgattgctttccttgagaccggtgctggaaagacacttatcgcgattcttcttattaaaagtgttcataaggatctgatgagccagaacagaaaaatgctctcggtgttcttggttcccaaagtgcctttggtttatcagcaagcagaagtgatccgtaatcaaacttgttttcaagttggacattattgtggtgagatgggacaggacttttgggattctcgaaggtggcaacgagagtttgagtctaagcaggttctagttatgacagcacaaattctgttgaatatactgagacacagtatcattagaatggaaacaattgatcttcttattctcgacgagtgtcaccacgctgtcaagaaacatccatactctttagtgatgtcagagttttaccatacaactcctaaagataaaagacctgccatctttggaatgactgcttcgcctgttaatttaaagggtgtttcaagccaagtagattgtgcgataaagatacgtaacctcgagaccaagttggattctacggtttgtactataaaagatcgaaaagaattagagaaacatgtgcctatgccttcagagatagtcgtcgagtatgacaaagctgctactatgtggtctcttcatgagacaataaagcaaatgattgcagctgttgaagaagcggcacaagcaagttcaaggaaaagcaagtggcaatttatgggggctagggatgctggagcaaaggatgaattgagacaggtttatggcgtctctgaaagaacggagagcgatggtgctgccaatttgattcataaacttagagctatcaattatactcttgctgaattgggtcaatggtgtgcttacaaggtgggacaatcattcttgtctgctttgcaaagtgatgagagggtgaatttccaagtcgacgtgaagtttcaagaatcatacctcagtgaggtggtgtcactcttgcaatgtgagcttctggaaggcgctgctgctgaaaaagtcgcggcggaagttggcaaaccagaaaatggtaatgcacatgacgagatggaggagggagagctccctgatgatcctgtggtctcgggaggggagcacgttgatgaagtaataggcgccgcagtggctgatgggaaagttactccaaaagtacaatcattgatcaaactactcctcaaatatcagcacacagctgattttcgagctattgttttcgttgagagggtggttgctgctttggttcttcctaaggtttttgcggagctgccttcgcttagttttatacggtgtgccagcatgattggacacaataacagccaggagatgaaatcatctcaaatgcaggatacaatttccaaattccgagatgggcatgtgacactgttagttgccacaagcgttgctgaggaaggacttgatattaggcaatgtaacgttgttatgcgtttcgaccttgcaaagacggtgctggcatacattcagtctcgtggccgggcaagaaagcctggatcagactacatactcatggttgagagaggaaatgtatctcacgcagcgttcctaaggaatgctaggaacagtgaggagacacttcgaaaagaagcaatagaaaggactgatcttagtcatctcaaagatacatcgagattaatctcaattgatgctgtgcctggtacagtttataaggtggaggcaactggtgccatggttagcttgaattccgcggttggtcttgtacatttctactgctctcagcttcctggtgacaggtatgcaatccttcgtcctgagtttagcatggagaagcatgaaaagcctgggggccacacggaatattcatgtaggcttcagcttccttgcaatgcaccgtttgaaatacttgagggtcctgtttgcagttcaatgcgtcttgcacaacaggctgtatgtttagctgcttgcaagaaactgcatgagatgggtgcatttaccgatatgctattaccggacaaaggaagtggtcaagacgctgagaaggctgaccaagatgatgaaggtgagcctgttcctggaactgctagacatagagagttctatcctgaaggtgtggcggatgtacttaagggagaatgggtttcatctggaaaggaagtttgtgagagctcaaagctattccatttatacatgtataatgtcagatgtgtagattttggctcttcaaaagatccattcctaagcgaagtttcagagttcgcgattctttttggcaatgagctggatgcagaggtattatcgatgtctatggatctttatgttgctcgggccatgatcactaaagcatctcttgctttcaagggatcacttgatattacagaaaaccagctatcatctctaaaaaagtttcatgtgaggttaatgagtatcgtgttggatgttgatgttgaaccctccacgacaccatgggatcctgcaaaggcctacctgtttgtccctgttactgacaatacgtctatggaacccataaaagggatcaactgggaattggttgaaaagattacgaaaaccacagcgtgggacaaccctcttcagagagctcgtcccgatgtatatctcgggactaatgagagaactcttggtggggacagaagggaatatgggtttggtaaacttcgtcacaacattgtatttgggcagaaatctcacccaacttatggtattagaggagctgttgcatccttcgatgttgtgagagcttctggattgttacctgtgagagatgcttttgagaaggaagtagaagaggatttatcaaaaggaaaattgatgatggctgatgggtgcatggttgcagaagatcttattgggaaaatagtgacagccgcacattccgggaagcggttttacgtagattcaatttgttatgacatgagtgcagaaacatctttccctaggaaagagggatatcttggtcccctagagtacaacacgtacgctgactattacaagcaaaagtatggagttgatttgaactgtaagcaacaacctttgattaaaggacgtggtgtttcgtattgcaagaaccttctttctcctcggtttgaacagtcaggtgaatctgagacagtccttgataagacatattacgtgtttcttccacctgaactatgcgttgtgcatccgctttcgggttcacttatccgaggtgctcagaggttaccctctataatgagaagagttgagagcatgttactcgctgttcaactcaaaaatttgattagttatcctattcccacatcaaagattcttgaagccttgactgccgcctcgtgccaggaaacgttctgctacgagagagctgagcttttaggagatgcgtatctaaaatgggttgttagtcgttttctgtttctcaagtatcctcaaaagcacgagggtcagcttacaaggatgaggcaacaaatggttagtaatatggttctttatcagtttgctctggttaaagggcttcagtcatatatccaggcggatcgattcgccccgtctaggtggtctgctcctggtgtgcctccggttttcgacgaggacacaaaagatggaggatcttcgtttttcgatgaagagcaaaaacctgtttccgaggaaaacagcgatgtgtttgaagatggggagatggaggatggtgaactagagggtgatttgagttcgtaccgagttttatctagcaaaacgttagctgatgttgttgaggctttgattggtgtttattacgtcgaagggggtaagattgcagctaatcatttgatgaaatggattgggattcacgtggaggatgatcctgatgaagtcgatggaacattgaaaaatgttaatgttccagagagtgtgctcaagagcatcgactttgttggtcttgagagagctcttaaatatgagtttaaagagaaaggtcttcttgttgaagctataacacatgcttcaagaccatcttcaggtgtttcgtgttaccagagattggaatttgttggtgacgcggtcttggatcatctcatcacaagacatctatttttcacatacacaagccttcctcctggtcggttaacagatcttcgagctgcagcggttaacaacgagaattttgctcgcgttgcggttaaacataaactccacttgtaccttcgtcacggttcaagcgccctcgaaaaacagattcgggaatttgtgaaggaggttcaaaccgagtcatcgaaaccggggtttaactcttttggtttgggagactgcaaagcaccaaaagttcttggagacattgttgaatctattgcaggtgctatttttcttgatagtggaaaagatacaactgctgcttggaaggtttttcaacctttgcttcagcccatggtgacaccagagacacttccaatgcatccggtgcgagagctacaagagcggtgccagcaacaagcagaagggttagaatacaaagcgagtaggagtggtaacacagcgactgtggaagttttcatcgacggtgttcaagttggagtagcgcaaaacccgcagaagaaaatggctcaaaagctagctgcgaggaacgcacttgcagctttgaaagagaaagaaatagcagaatcaaaggagaagcatatcaacaacggtaatgcgggagaggatcaaggcgagaatgagaatgggaacaagaagaatgggcatcagccgtttacgagacaaacgttgaatgatatttgtttgaggaagaattggccaatgccttcttacagatgtgtgaaagaaggaggaccggctcatgcaaagagatttacgtttggggtaagagttaatacgagcgatagaggatggaccgatgagtgtattggcgagccaatgccgagtgttaagaaagctaaggattcagctgcggttcttctacttgagcttttaaataaaactttttcttga

SEQ ID NO: 62 - DNA, 5733 bp, Arabidopsis thaliana
  misc_feature 1..5733: note = ORF of protein Dcl1
  source 1..5733: mol_type = unassigned DNA
atggtaatggaggatgagcctagagaagccacaataaagccttcttattggctagatgcttgcgaggacatctcttgtgatcttatcgatgatctcgtgtctgaatttgatccttcctctgttgctgtcaatgaatccactgatgaaaacggcgtcatcaatgattttttcggtgggattgatcacattttagatagtatcaagaacggtggaggcttaccaaacaatggcgtttctgataccaattctcaaatcaacgaggttactgtaactcctcaggttattgctaaggagacagtgaaggagaatgggttgcaaaagaatggcggtaagagagacgaattctcgaaagaggaaggagacaaggataggaagagagctagggtttgtagttatcagagtgaaaggagtaacctttcaggtagagggcatgttaataattctagggagggagataggtttatgaataggaaacgtactcgtaattgggacgaggcgggtaacaataagaagaaaagggaatgtaacaattacagaagagatggtagagatagagaagttaggggttattgggagagggataaagttggttccaatgagttggtttataggtcagggacttgggaagctgatcatgaaagagatgttaagaaagtgagtggtggaaaccgcgaatgcgatgtcaaggcagaggagaacaagagtaagcctgaagaacgtaaagagaaggttgtggaagagcaagcaaggcgataccagttggatgttcttgaacaagctaaagcgaaaaacacgattgctttccttgagaccggtgctggaaagacacttatcgcgattcttcttattaaaagtgttcataaggatctgatgagccagaacagaaaaatgctctcggtgttcttggttcccaaagtgcctttggtttatcagcaagcagaagtgatccgtaatcaaacttgttttcaagttggacattattgtggtgagatgggacaggacttttgggattctcgaaggtggcaacgagagtttgagtctaagcaggttctagttatgacagcacaaattctgttgaatatactgagacacagtatcattagaatggaaacaattgatcttcttattctcgacgagtgtcaccacgctgtcaagaaacatccatactctttagtgatgtcagagttttaccatacaactcctaaagataaaagacctgccatctttggaatgactgcttcgcctgttaatttaaagggtgtttcaagccaagtagattgtgcgataaagatacgtaacctcgagaccaagttggattctacggtttgtactataaaagatcgaaaagaattagagaaacatgtgcctatgccttcagagatagtcgtcgagtatgacaaagctgctactatgtggtctcttcatgagacaataaagcaaatgattgcagctgttgaagaagcggcacaagcaagttcaaggaaaagcaagtggcaatttatgggggctagggatgctggagcaaaggatgaattgagacaggtttatggcgtctctgaaagaacggagagcgatggtgctgccaatttgattcataaacttagagctatcaattatactcttgctgaattgggtcaatggtgtgcttacaaggtgggacaatcattcttgtctgctttgcaaagtgatgagagggtgaatttccaagtcgacgtgaagtttcaagaatcatacctcagtgaggtggtgtcactcttgcaatgtgagcttctggaaggcgctgctgctgaaaaagtcgcggcggaagttggcaaaccagaaaatggtaatgcacatgacgagatggaggagggagagctccctgatgatcctgtggtctcgggaggggagcacgttgatgaagtaataggcgccgcagtggctgatgggaaagttactccaaaagtacaatcattgatcaaactactcctcaaatatcagcacacagctgattttcgagctattgttttcgttgagagggtggttgctgctttggttcttcctaaggtttttgcggagctgccttcgcttagttttatacggtgtgccagcatgattggacacaataacagccaggagatgaaatcatctcaaatgcaggatacaatttccaaattccgagatgggcatgtgacactgttagttgccacaagcgttgctgaggaaggacttgatattaggcaatgtaacgttgttatgcgtttcgaccttgcaaagacggtgctggcatacattcagtctcgtggccgggcaagaaagcctggatcagactacatactcatggttgagagaggaaatgtatctcacgcagcgttcctaaggaatgctaggaacagtgaggagacacttcgaaaagaagcaatagaaaggactgatcttagtcatctcaaagatacatcgagattaatctcaattgatgctgtgcctggtacagtttataaggtggaggcaactggtgccatggttagcttgaattccgcggttggtcttgtacatttctactgctctcagcttcctggtgacaggtatgcaatccttcgtcctgagtttagcatggagaagcatgaaaagcctgggggccacacggaatattcatgtaggcttcagcttccttgcaatgcaccgtttgaaatacttgagggtcctgtttgcagttcaatgcgtcttgcacaacaggctgtatgtttagctgcttgcaagaaactgcatgagatgggtgcatttaccgatatgctattaccggacaaaggaagtggtcaagacgctgagaaggctgaccaagatgatgaaggtgagcctgttcctggaactgctagacatagagagttctatcctgaaggtgtggcggatgtacttaagggagaatgggtttcatctggaaaggaagtttgtgagagctcaaagctattccatttatacatgtataatgtcagatgtgtagattttggctcttcaaaagatccattcctaagcgaagtttcagagttcgcgattctttttggcaatgagctggatgcagaggttgtattatcgatgtctatggatctttatgttgctcgggccatgatcactaaagcatctcttgctttcaagggatcacttgatattacagaaaaccagctatcatctctaaaaaagtttcatgtgaggttaatgagtatcgtgttggatgttgatgttgaaccctccacgacaccatgggatcctgcaaaggcctacctgtttgtccctgttactgacaatacgtctatggaacccataaaagggatcaactgggaattggttgaaaagattacgaaaaccacagcgtgggacaaccctcttcagagagctcgtcccgatgtatatctcgggactaatgagagaactcttggtggggacagaagggaatatgggtttggtaaacttcgtcacaacattgtatttgggcagaaatctcacccaacttatggtattagaggagctgttgcatccttcgatgttgtgagagcttctggattgttacctgtgagagatgcttttgagaaggaagtagaagaggatttatcaaaaggaaaattgatgatggctgatgggtgcatggttgcagaagatcttattgggaaaatagtgacagccgcacattccgggaagcggttttacgtagattcaatttgttatgacatgagtgcagaaacatctttccctaggaaagagggatatcttggtcccctagagtacaacacgtacgctgactattacaagcaaaagtatggagttgatttgaactgtaagcaacaacctttgattaaaggacgtggtgtttcgtattgcaagaaccttctttctcctcggtttgaacagtcaggtgaatctgagacagtccttgataagacatattacgtgtttcttccacctgaactatgcgttgtgcatccgctttcgggttcacttatccgaggtgctcagaggttaccctctataatgagaagagttgagagcatgttactcgctgttcaactcaaaaatttgattagttatcctattcccacatcaaagattcttgaagccttgactgccgcctcgtgccaggaaacgttctgctacgagagagctgagcttttaggagatgcgtatctaaaatgggttgttagtcgttttctgtttctcaagtatcctcaaaagcacgagggtcagcttacaaggatgaggcaacaaatggttagtaatatggttctttatcagtttgctctggttaaagggcttcagtcatatatccaggcggatcgattcgccccgtctaggtggtctgctcctggtgtgcctccggttttcgacgaggacacaaaagatggaggatcttcgtttttcgatgaagagcaaaaacctgtttccgaggaaaacagcgatgtgtttgaagatggggagatggaggatggtgaactagagggtgatttgagttcgtaccgagttttatctagcaaaacgttagctgatgttgttgaggctttgattggtgtttattacgtcgaagggggtaagattgcagctaatcatttgatgaaatggattgggattcacgtggaggatgatcctgatgaagtcgatggaacattgaaaaatgttaatgttccagagagtgtgctcaagagcatcgactttgttggtcttgagagagctcttaaatatgagtttaaagagaaaggtcttcttgttgaagctataacacatgcttcaagaccatcttcaggtgtttcgtgttaccagagattggaatttgttggtgacgcggtcttggatcatctcatcacaagacatctatttttcacatacacaagccttcctcctggtcggttaacagatcttcgagctgcagcggttaacaacgagaattttgctcgcgttgcggttaaacataaactccacttgtaccttcgtcacggttcaagcgccctcgaaaaacagattcgggaatttgtgaaggaggttcaaaccgagtcatcgaaaccggggtttaactcttttggtttgggagactgcaaagcaccaaaagttcttggagacattgttgaatctattgcaggtgctatttttcttgatagtggaaaagatacaactgctgcttggaaggtttttcaacctttgcttcagcccatggtgacaccagagacacttccaatgcatccggtgcgagagctacaagagcggtgccagcaacaagcagaagggttagaatacaaagcgagtaggagtggtaacacagcgactgtggaagttttcatcgacggtgttcaagttggagtagcgcaaaacccgcagaagaaaatggctcaaaagctagctgcgaggaacgcacttgcagctttgaaagagaaagaaatagcagaatcaaaggagaagcatatcaacaacggtaatgcgggagaggatcaaggcgagaatgagaatgggaacaagaagaatgggcatcagccgtttacgagacaaacgttgaatgatatttgtttgaggaagaattggccaatgccttcttacagatgtgtgaaagaaggaggaccggctcatgcaaagagatttacgtttggggtaagagttaatacgagcgatagaggatggaccgatgagtgtattggcgagccaatgccgagtgttaagaaagctaaggattcagctgcggttcttctacttgagcttttaaataaaactttttcttga

SEQ ID NO: 63 - DNA, 1713 bp, Amaranthus hypocondriacus
  misc_feature 1..1713: note = ORF of protein Dcl1
  source 1..1713: mol_type = unassigned DNA
atgagacgggtggaaagcatgcttcttgctattgaacttaagaatatcatcaaatatcctgtccctgcttctaagattctggaagctttgactgcagcctcttgtcaagaaacattttgttatgaaagagccgaactcctaggagatgcttatttgaaatgggtcgttagtagatatctttttcttaaatatccccagaaacacgagggtcagctcacacggatgagacaacaaatggtgagcaatttggtcttataccatcatgcactggagaaagggcttcagtcgtatatccaagcagatcgctttgctccgtctaggtgggctgctcctggtgttcctcctgtttttgatgaggacaccaaagaagaggaatcatctttatttgatcaggaaaaaactaatttaaagattgatcatgaaagagtcaataattatgatgttgacgatgatgaagaaatggaagatggtgaacttgaaggtgattcaagctcttacagggtgctttctagcaaaacattggctgatgttgttgaagcactaattggagttcattacgtcgaaggcggaaagaacgccgctaaccacttcatgaaatggattggaattgatatagagttcaatcctgaagatattgagtgtccaaagaggccaattatggttgcagaaggtgttcttagaagtgtggattttgatacattagaagctgctttaaatattaagttcaaagataggactttgttgattgaagcaattacgcatgcttcacgcccgtcttctggagttgcatgctaccagcggcttgagtttgtgggtgatgcagttttggatcatctaataactaggcatctgttcttcaggtatacagatctgcctccaggccgtctgacagatctgcgtgctgcagctgttaacaatgaaaattttgcccgggttgccgtcaaacataagcttcaccagcatttgcggcatgggtctactgccctggagaagcagatacgggattttgtgaaggaagttcaagaggagttgttaaaacctggttttaattccttcggattaggagattgtaaagccccgaaagtacttggtgatattgttgaatcgattgctggagctatttttcttgatagtggacatgacgcaacagttgtttggagagtttttgagcctttgcttcaaccaatggttactcccgatacacttcccatgcatcctgtccgagagttgcaagaacgatgtcagcagcaggctgaaggcctggaatataaggccactagagaagggaatttagccactgttgaagtttacgtcaacggaatccaagttggagtcgctcataatccacagaaaaagatggctcagaaattggctgctcggaatgctctgcttgttttgaaggagaaggaaactgctgaagctaaggtcaaggctgaggagagcgagaaaaaaaacaagaatggggtagtaacattcacaaggcagacactgaatgatatatgcttacgcagaaattggcccatgccactctataggtgtgtgaatgaaggaggccctgctcatgccaagcgattcacatttgcagttcgggttaataccagtagtaaaggttggaccgaggaatgcattggagagcctatgcctagtgttaagaaggccaaagactcggcagcggctcttcttttggaactgttgaacaaacgatattcctaa

SEQ ID NO: 64 - DNA, 5793 bp, Brachypodium distachyon
  misc_feature 1..5793: note = ORF of protein Dcl1
  source 1..5793: mol_type = unassigned DNA
atggctggaggaggcggcggcgccggggccggcgagcacgcggcggcggcctactggtacgacgcgtgcgaggacggggcgtcgctgctgtgcggcatcgacttcgccgcgtccgccgacttcgacccgggcctcatgcccgccatggacagcggcgtcgacgacggcttcgtcgccgagatcgaccggatactcgagagcatcaacgcggagagcacccccgcgccgccgccgccgcagcagcagcaagcgcttgtggacccctcgccgcagccgccggtggagcaagggcaggaggctgctgtggccgtggccgacaatgccgtggaggtggtgggctcagggcagaggatccagggcgccgagacgaggaaggagatgaagaaggagtcgcacagcagttccacgaatggcagttccgcgaatggcggggaccggagagaccagaagcgtccgcccctcgcctctggggtcgccagcgggccgcgccctgattggcggcggcggccgatgccaccaccgccctcgcgtggattggaggatcggcgtgggaggcgcgacttcgacaggcctcgcaagcgcgaccgcgacggccactctggctccgagcatcaccggcgcgaggctcggggattttgggagcgtgaccgcggcggtaagatggtgttccggccgggcacttgggagaaggagtctgaccgcgaggcaaagcgtgccaggacacaggacgggggttctgtggagaagaaagcagaggcggatagggcgggggcagcgcagagggcgaagcctgtcgctgaggagcaggcgaggcattaccagttggaggtgcttgagcaagcaaagagcaggaacaccatagcctttctcgagaccggcgccgggaagactctcattgctgtgctgctcatcaaaagtatatgtgacaaaatgctcaaggagaacaagaagatgcttgctgtcttcttggttcccaaggtgcctcttgtgtaccagcaagcggaagtgatacgcgagaggactggctatcggattgggcattactgcggagagatgggtcaggatttctgggacgctagaaagtggcagcgtgagtttgattcaaaacaggtactagttatgacagctcaaattttgttaaacatcctcaggcacagtattattaaaatggatgcgatacatcttttaattttagacgagtgccatcatgcagtgaaaaaacatccttattctctagtgatgtcagaattttatcacacgactccaaaggataagagaccagttgtttttggtatgacagcttctcctgtcaacctaaagggagtcactagccaagaggattgtgccatcaagataagaaatcttgagagtaaacttgattctgtagtttgtactataaaagatcggaaggagcttgagaaacatgtccccatgccttttgaagttgttatccattacgacaaagcagccactctttgttccttgcacgagcggatcaaacaaatggaagctgcagttgaagaagcggcactctctagctctaagagaaccaggtggcagtttatgggagcaagggatgctgggtctagagaggagctgcgccttgtctatggtgtttctgagcgtacagaaagtgatggtgcagctaatttgatccagaagttgagagctataaattatggtttaggtgaactaggacaatggtgtgcttacaaggttgcacagtccttcttgacagcgttacaaaatgatgaaagggcaaactatcaggttgatgtaaagtttcaggagtcctatttaaagaaggttgttgatttgttacactgccagttgactgaaggagctgcaatgaaaagcgagactaatgatgttgagatgcatagtactgaaaaccataactcaagtgaacttgaggatggggagctacctgacagccatgctgtttcggtaggtgaacatgtagatgaagtcatcggagctgccgtggcagatgggaaagttacccctagggtgcaggctttgatcaaaatccttctcaagtaccagcataccgaggatttccgtgctatcatctttgtggagcgggttgtcacggcattagttcttcctaaggtgtttgcggagcttccatctttaagctttattcgatgcgctagcttgataggccataacaacaatcaagagatgcgcacaagccaaatgcaggatacaattgccaaatttcgcgatggccgtattacattgttagttgcgactagcgtcgcagaggaagggcttgatattcgacagtgcaatgttgtcattcgctttgaccttgcgaagactgtcttggcctacatacagtctcggggtcgtgcaaggaagcctggatctgactacatattgatggtcgaaagggcaaatttatcacatgaaactttcttgaggaatgctcgaaatagtgaggagacgttaagaaaagaagctatagaaagaactgatctcagtcatcttgatggcattcctatgttaaatcctatccatacatctcctgattcgatgtatcaggtggaatcaacaggtgctgttgtcagcttgaattctgcagttggactcatacatttttactgttcacagcttccaagtgacaggtactcaattcttcgtccagaatttattatgcaaaaacatgagaaaccagggggttccgtggaatattcttgcaaacttcaactcccctgcaatgcaccatttgagaaacttgagggtcctatatgcagctcgattcgtctggcacagcaagctgtttgtttggctgcctgtaagaaactacatgagatgagtgccttcacagatatgcttttacctgatagaggaattggggaaggagaaaagactgaacagaatgatgaaggtgatccagtccctggaacagcacggcatagagagttttatcctgaaggtgttgcggaagttctgagaggagaatggattttatctggaagagattgctgccaaagctctcaatttattaagttgtatatgtattacgtgaactgtgtggatgtggggacatctaaggaccccttccttgcacaactttcaaatttttctatagtttttggcaatgagctggatgcagaggttttatcgatgacaatggatctctttgttgccaagacaatgataacaaaggcatctcttgtattccatggaccaattgaaattacagagagacagttggtattgctgaagagctttcatgttaggctcatgagtatagtccttgatgttgatgtcgatccctcaactacaccttgggatccagcaaaagcgtacctctttgtccctgtaggagctgagaagtgtacagatgctttcagagagatcgattggactttagtcaacagcattgtgaacactgatgcctggaataatccacttcagaaggcacgcccagatgtctacctaggcaccaatgagcggacacttggtggagataggagagagtatgggtttgggaaattacgtaatggaaccacttttggacagaaggcacatccgacttatggtattagaggagccattgctgagtttgatgttgtcaaagcatctggattagttcctgctcgtgacagagggtttcttaatgactctcagaaacacgggaagttgttcatggcagattcatgctgggatgctaaagatcttgctggaatggttgtcactgctgcccactcaggaaagaggttctatgtggactctatttgttataacatgaatgcagaaaattcatttcctaggaaggaaggctacctggggcctttggaatatagctcgtatgctgattactacaagcagaaatacggcgtggagctagtgtatacgaaacaacctcttatacgtgcacgtggtgtttcgtactgcaagaatctcctgtctcctagatttgagcattctgaagctaaagaaggtgtcaacttttctgagaaccatgacaaaacatactatgtgtatctaccacctgaactgtgcgttgtgcaccctcttcctggatcacttgttcgtggagctcagaggttgccttcaataatgagaagggttgagagcatgcttcttgcagttcagttgaaggaaataattgattatcctgttcccgcaacgaagatattagaagccttgactgctgcatcatgccaggaaacgttttgctatgaaagagcagagctactgggtgatgcatacctgaaatgggttgtgagtagattcctttttctaaaatatcctcagaagcatgagggacagcttacgaggatgcggcagcagatggttagtaacatggttctgtaccaattcgctctgaataaaactcttcagtcgtacattcaagcagatcgttttgccccatcaagatgggcagctccaggagtgctacctgtatttgacgaggagacaagagaatctgaaccatccatctttggtgaggaaccagttcttggtaatgaagtacagaaggattttgatgacgattacgcagatagcatccaagaagacggtgaaattgatggagattctggctgctatcgtgttctctcaagcaaaactttagcagatgttgttgaagcacttattggggtctattatgtagcaggagggaaaattgctgcaaaccacctgatgaagtggattgggattcatgcagagctggatcctcaagagatccctcctccgaagatatataatatacctgatagcataatgagaagcatcaactttgacaccttagaaagcatcatgggtatcaaattccagaataaaggttttctggttgaggctataacgcatgcatcgaggccgtcttctggtgtttcctgttaccagcggttggaatttgttggtgatgctgtgttggatcatctcatcacaaagcatttgttttttacatatactgatctacctcctggtcgcttaaccgacttgagagctgcggctgttaataatgagaactttgcaagggttgcagttaagcataagctgcatgtacatcttcgccatgggtcatctgcgctggaaacacagattcgggagtttgtgaaggatgtccgagaagagatttcaaaatcaggtttcaattcttttgggcttggggactgcaaggctcctaaagttcttggagacattattgagtctattgctggtgcaatatttctcgatagtgggtatgacacctcggcagtctggaaggtttttcaaccgcttcttgatcctatggtaacgccagaaacccttcccatgcatccaattagggagctccaagaacgctgccagcagcaagctgaaggcttagaatacaaagcatcccgagcaggcaatgtagctactgttgaagtcttcgtggatggtatccagattggtgtagctcagaacccgcagaagaagatggcacagaagttggcggcgaggaatgcacttgtggtcctgaaggagaaggaaagcgcagaagccaaggagaccgaaaaggatgacgataagaagaacggtgctcatgtgttcaccaggcagaccctgaacgacatctgcttgagaaggcagtggccgatgccacaatacaggtgcataaacgagggcggccctgctcatgctaaaagatttgtctacgcggtaagggtgaacacatctgatcgtggatggaccgacgaatgcattggagagccaatgcctagtgtgaagaaggccaaggactctgctgccgttcttctccttgagcttctgaatggaagtttccctgacaagtctgacggcaaaaagccctag

SEQ ID NO: 65 - DNA, 5844 bp, Glycine max
  misc_feature 1..5844: note = ORF of protein Dcl1
  source 1..5844: mol_type = unassigned DNA
atggaggatgggagcagggttccggcgggggatgacccctcttactggctggatgcgtgtgaggacatatcgtgcgatgatttcattgattttgatgtctcttccatcgtctctgaccagcccgacaatccttccaatcaggacttcttcggtggcattgataagattttggacagcatcaagaacggtgctggtctccctctcaatcatgctgttgaaccacccaataataatggcactgcagcaggagaagtgtgcttgccttccaatgccactcttgaggatggtgctcctgctgctgatgcttttgaccattctggtggcgttgctcggagtaacggctccagtaagctgtctaatgggaacgaaacaggagttttggttaattattctcaggaaagaggggcaccccctctgaatggaggccacgattttgatggtgaagaaaggtgcagcaaaagggcttggcttggcggctacaataatgagaggccttattattgtagagggaattatcaaggcaaggagagggagaggtgttttaataataataacaggaagaggccacggggtgatagggatgaaattgacaggaaagataaggatggtggtggaaggaaaagagaacattatggtgctgttgccaggagggatgttagagatagggattgtagggatagagaaactaggggttactgggagagggataagtcaggttccactgatatgatctttcgcactggcgcttgggaacctgatcataatcgagatgacaaaatggtgattgacaccaaactggagaactatggaaagcttgataagaagtccgaggatgctattgagagggttcctgaggaaaaagctagacagtatcagttagatgttcttgagcaatcaaaaaggaaaaataccatagcttttcttgaaactggagcagggaaaaccttaattgctgttcttctcattaaaagtatacaagatagtttgcagaagcaaaataagaaaatgcttgcagtatttttagttccaaaagttccacttgtttaccagcaagctgaagtaattcgtgagcggactggttatcaagtaggccactattgtggagaaatggggcaggatttctgggatgctcgaaggtggcagcgtgaatttgacaccaaacatgttttagtcatgactgcccaaattcttttaaacattttgaggcatagcataataaaaatggaagcaatcaatctcttgattctggatgagtgccaccatgcagtgaagaaacacccatattcactggtgatgtccgagttctaccatacaacacccaaagagaacaggccatctgtatttggaatgactgcttctcctgttaacttgaagggtgtctccagccaagtagattgtgcaataaaaattcgtaatcttgaaagtaagctagattctatagtttgcaccattaaagatcggaaggagctggagaaacatgtgccgatgccctctgaagttgtggtggagtatgataaagctgccagtttatgttatctacacgagcagataaagcagatggaggttgaagttgaagaagctgcaaaatatagttcaaggagaagcaaatggcagtttatgggagctagagatgctggagctaaagaagagctgcgccaagtatatggcgtttctgaaagaacagaaagtgatggagctgcaaacttaattcagaagttgagagctgttaattatgcacttggtgaactgggacaatggtgcgcttataaggttgcactatcctttttagcagctttgcaaaatgatgaaagggccaactaccagcttgatgtcaagtttcaggaaacttatttgagtaaagttgtctcgcttctgaaatgccaactgtcagagggtgcagcttctgacaaaaatgctggtattgatgactcagagaatggtgcagctcaaagtgggtctgaacatgaagagatggaagaaggagagcttccagacagtcatgttgtctctggcggggagcacgtggatgttattataggagctgctgtggctgatggcaaggtgacccccaaggtgcaggcactaatcaaaatacttctcaagtatcagcatacagaagatttccgtgcaatcatctttgttgagcgtgttgtgtctgcattagttctgcccaaggtctttgcagagcttccatcacttagttttgtgaagtgtgcaagtttgattggtcataataacagtcaggaaatgcgaacttaccaaatgcaggatacaattgccaaattccgtgatggacgggtcacattgttggttgccactagtgttgctgaagaaggacttgatatccgtcaatgcaatgttgttattcgctttgaccttgcaaagactgttctggcatatattcagtccaggggacgtgctagaaagcctggatctgattacatcctgatggttgagagggacaatttgtcacatgaagcattcctaaggaatgccaagaacagtgaggaaactttgcgcaaagaagcaatagagagaacagaccttagtcatctgaaggacacttcaagattgatttctgttgacacccggcccggaacagtttaccaggtgaagtcaaccggtgcagttgtgagcctaaattctgctgtaggtcttatccacttttactgctctcagttacctagtgacagatattcaattcttcgccctgagtttattatggagaggcatgaaaaaccaggaggtcccacggaatattcttgcaagcttcaactaccgtgtaatgcaccgtttgaaaatcttgagggtccaatatgcagttctatgcgtctggcacaacaggctgtttgtctggctgcttgcaagaaactgcatgagatgggtgcattcactgacatgctattgcctgataaaggaagtgggggagaaagagaaaaggatgaacaaactgatgaaggagatccacttccagggactgctagacatagggagttctaccctgaaggtgtggctgacatactgaagggagagtggatcttatctgaaaaagatgcttgcaacaactacaaattacttcatctttatatgtatgccgtgaagtgtgaaaatcttggccattcaaaggatccgttcttgactcaagtttcaaattttgcagtactttttggcaatgagctggatgcagaggtgttatcgatgtcaatggatctatttatcgctcgaactgtgactacaaaggcatctcttgtctttagtgggttgataaatatcactgagagtcagctggcatccctgaaaagctttcatgtaagattaatgagcattgtcttggatgtggatgttgaaccatctaccactccttgggatcctgcaaaggcatatttgtttgtcccaatggttggcgataagtccgtagatcctatgaaccaaattgactggcatctggttgagacaataattggagctgatgcatggaagaatcccctccagaaagctcgaccagatgtttaccttggtactaatgagagaacattaggtggagacagaagggaatatggatttgggaaattgcgtcatggcatggcttttgggcaaaaatcacatcctacctatggaatcagaggagctgtggcccagtttgatgtggtgaaagcttcaggattggttcctaacagagacgccatgcaaacacaaaagcacatcaatatgactaccaatggaaaattaatgatggcggatatctgtactaatgcagaggatctggttgggagaattgtaactgctgctcattcagggaagaggttttatgttgattcaatatgctatgacatgtcagcagagaactctttccctaggaaagaaggttatcttggtcctctggagtacagttcatatgctgattactacaagcaaaagtatggggttaatttgatttacaagcagcaacctcttataagagggcgtggtgtatcatactgcaagaatcttctgtctcctcgatttgagcacagtgaagctcatgaaggtgaatctgaagagatacatgacaaaacttattatgttttccttcctcctgagctatgcctcgtacacccactgcctggatcacttgtccgtggtgcccagaggttgccttcgataatgaggagggttgaaagtatgctacttgcagttcagcttaagaatatgataaattatcctgtcctggcttcaaagatcttgggagccttgactgccgcctcttgccaggagacattctgctatgaaagggcagagcttcttggagatgcttacctgaaatgggttgttagtcgatttcttttcctaaaatatccacagaaacatgaaggtcaacttactaggatgagacaacaaatggtcagtaacatggtattatatcagtatgcattaagtaaagggcttcaatcatatattcaagcagatcgttttgctccatccagatgggcagctcctggggtgctgcctgtctttgatgaggataccaaggatggggagtcatctttgtttgaccaggagcgatccatttccaagatcgagaggatggattgtcacacgaatggatatgaggatgagatggaagatggtgagcttgagagtgattcaagttcttacagagttctatccagtaagacacttgcagatgttgtggaagcactgattggggtatattatgtggaaggtggtaagaatgctgccaaccaccttatgaaatggattggcattcaaatagagtttgatcctgatacaatggagtgtacaaaaaagccatttaatgttcctgacagcatacttaggagtgttgattttgatgctttagaaggtgctttaaatatgaagtttaacgacagggggctgctggtagaatccataactcatgcctcccggccatcttcaggagtatcctgttatcagcggttggagtttgttggcgatgctgtcttggatcatcttattaccaggcacttgtttttcacttacacaaatctgccaccgggacgtctgactgaccttcgggctgctgctgtaaacaatgaaaattttgcgcgtgttgcagttaagcacaaccttcatgtgcatctcagacatggatccagtgccctagaaaaacagattaaagaatttgtgaaggaagttcaagtcgaattgtcaaagccaggttttaattcctttggtctgggagactgtaaagcccccaaggtccttggtgacattgttgaatctattgcaggtgccatttttcttgacagtgggagggacactactgttgtttggaaggtttttcagcctcttttgcatcctatggttaccccagagactctgccgatgcatcctgtgcgtgagctgcaagagcgatgccagcagcaagctgaaggcttagagtataaagcaagtcgtattggcaatttggccactgttgaagtattcattgatggggtccaagtaggggctgctcaaaatccacagaaaaaaatggcgcagaaattagctgcaagaaatgcccttgctgctttgaaagagaaagaggtgggaaaaactcaggagaagaatgatgaaaacgggaaaaagaatggaaaccagacatttactaggcaaacattgaatgatatatgtttacgaagaaattggcctatgccattctacaggtgtgtgaatgagggtggtccagcacatgcaaagaggtttacatttgcggtgcgtgtcaatactactgatagaggatggacggatgaatgtgttggagagccaatgccaagtgtcaagaaggccaaagactcagctgctgttcttctcttagaactactgaacaaattatactcgtga

SEQ ID NO: 66 - DNA, 5838 bp, Glycine max
  misc_feature 1..5838: note = ORF of protein Dcl1
  source 1..5838: mol_type = unassigned DNA
atggaggacgggagtagggttccggcgggggatgacccgtcttactggctggatgcgtgtgaggacatatcgtgtgatgatttcattgattttgatgtctcttccatcgtcgtctctgaccagcctgacaacccttccaatcaggacttcttcggtggcattgataagattttggacagcatcaagaacggtgctggtctccctctcaatcatgctgctgctgaaccacccagtaatgtcactgcagcagcatcaggaggagcagaagtgtgcttgccttccaatgccactccggaggattcttttgaccattctggtggcgctgctctgagtaacggctccagtaagcagtctaatgggaacgaaacgggagttttggttgattattctcaggaaagggggacacccactctgaatggaggccttgattttgatggtgaagaaaggtgcagcaaaagggctcggcttggtggctacaataatgatcggccttatcatggtagagggaattatcaaggcaaggagagggagaggtgttttagtaataacaggaagaggccgcgggggggtagggatgaaattgataggagggataaggatggtggtggaaggaaaagagaacattgtggtgctgttggcaggagggatgttagagatagggattggagggatagagaaactaggggttactgggagagggataagtctggttccactgatatggtctttcgcacgggtgcttgggaacctgattgtaatcgagaagacaaaatggcgattgacatgaaactggagaaaaatggaaaccttgataagaagtccgaggaggctaaggagagggttcctgaggaaaaagctagacagtatcagttggatgttcttgagcaggcaaagaggaaaaatactatagcttttcttgaaactggagcagggaaaaccttaattgctgttcttctcattaaaagtatacaggagagtttgcataagcaaaataagaaaatgcttgcagtatttttagttccaaaagttccacttgtttaccagcaagccgaagtaattcgtgagcggactggttatcaagtaggccactattgtggagaaatggggcaggatttttgggatgctcgaaggtggcagcgtgaatttgacaccaaacatgttttagtcatgactgctcaaattcttttgaacattttgaggcatagcataataaaaatggaagcaatcaatctcttgattctggatgagtgccaccatgctgtgaaaaaacacccatattcactggtgatgtctgagttctaccatacaacacccaaagagaacaggccatctgtatttggaatgactgcttctcctgttaacttgaagggtgtctccagccaagtagattgtgcaataaaaattcgtaatcttgaaagtaagctagattctatagtttgcaccattaaagatcgtaaggagctggagaaacatgtaccgatgccctctgaagttgtggtggagtatgataaagctgccagtttatgttatctacacgagcagataaagcagatggaggttgaagttgaagaagctgcaaaatgtagttcaagaagaagcaaatggcagtttatgggagctagagatgctggagctaaggaagagctgcgccaagtatatggtgtttctgaaagaacagaaagcgatggagctgcaaacctaattcagaagttgagagctgttaattatgcacttggtgaactgggacaatggtgtgcatataaggttgcacaatcctttttagcagctttgcaaaatgatgaaagggccaactaccagcttgatgtcaagtttcaggaaacttatctgagtaaagttgtttcacttctgaaatgccaactgtcggagggtgcagtttctgacaaaaatgctggtattgatgactcagagaatggtgcagttcaaagtggctctgaacatgaagagatggaagaaggagagcttccagacagtcatgttgtctctggcggggagcatgtggatgttattataggagctgctgtggctgatggcaaggtgacccccaaggtgcaggcactaatcaaaatacttctcaagtatcagcatacagaagatttccgtgcaatcatctttgttgaacgtgttgtgtctgcattagttctgcccaaggtctttgcagagcttccatcacttagttttgtgaagtgtgcaagtttgattggtcacaataacagtcaggaaatgcggacctaccaaatgcaggatacaattgccaaattccgtgatgggcgggtcacattgttggttgccactagtgttgctgaagaaggacttgatatccgtcaatgcaatgtcgttattcgctttgaccttgcaaagactgttctggcatacatacaatccaggggacgtgctagaaagcctggatctgattacatcctgatggttgagagggacaatttatcacatgaagcattcctaaggaatgccaggaacagtgaggaaactttgcgcaaagaagcaatagagagaacagaccttagtcatctgaaggacacttcaaggttgatctctgttgacacccggcctggaacagtttaccaggtgaagtcaactggggcagttgtgagcctaaattctgctgtaggtcttatccacttctactgctctcagctacctagtgacagatattcaattcttcgccctgaatttattatggagaggcacgaaaaacctggaggtcccacagaatattcttgcaagcttcaactaccatgtaatgcaccctttgaaaatcttgagggtccaatatgcagttctatgcgtctggcacaacaggctgtttgtctggctgcttgcaagaaactgcatgagatgggtgcattcactgacatgctattgcctgataaaggaagtgggggagaaaaagaaaaggatgaacaaactgatgaaggagatccacttccagggactgctagacatagggagttctaccctgaaggtgtggctgacatactgaagggagaatggatcttatctggaaaagatgcttgcaacaactccaaattgcttcatctttatatgtatgctgtgaagtgtgaaaatctaggccattcaaaggatccgttcttgactcaagtttcaaattttgcagtactttttggcaatgagctggatgcagaggtgttatcgatgtcaatggatctatttatcgctcgaactgtgactacaaagtcatctcttgtctttagggggttgataagtatcactgagagtcagctggcatccctgaaaagctttcatgtaagattaatgagcattgtcttggatgtggatgttgaaccatctaccactccttgggatcctgcaaaggcatatttgtttgtcccaatggttggcgataagtctgtagatcctacgaaccaaattgactggcatctggttgagacaataattggagctgatgcatggaagaatcccctccagaaagctcgaccagatgtttaccttggtactaatgagagaacattaggtggagacagaagggaatatggatttgggaaattgcgtcatggcatggcttttgggcaaaaatcacatcctacctatggaatcagaggagctgtggcccaatttgatgtggtgaaagcttcaggattggttcctaacagagatgccatgcaaacacaaaagcacatcaatatgactaccaatggaaaattaatgatggcggatacctgtactaatgcagaggatctgattgggaaaattgtaactgctgctcattcagggaagaggttttatgttgattcaatacgctatgacatgtcagcagagaactctttccctaggaaagaaggttatcttggtcctctggagtacagttcatatgctgattactacaagcaaaagtatggggttgatttgatttacaggcagcaacctcttataagagggcgtggtgtatcatactgcaagaatcttctgtcccctcgatttgagcacagtgaagctcatgaaggtgaatccgaagagacacatgacaaaacttattatgtttttcttcctcctgagctatgccttgtacacccattgcctggatcacttgtccgtggtgcccagaggttgccttcaataatgaggagggttgaaagtatgctacttgcagttcaacttaagaatatgataaattatcctgtccaggcttcaaagatcttggaagccttgactgccgcctcttgccaggagacattctgctatgaaagggcagagcttcttggagatgcttacctgaaatgggttgttagtcgatttcttttcctaaaatatccacagaaacatgaaggtcaacttactaggatgagacaacaaatggtcagtaacatggtattatatcagtatgcattaagtaaagggcttcaatcatatattcaagcagatcgttttgctccatccagatgggcagctcctggggtgctgcctgtctttgatgaggataccaaggatggggagtcatctttgtttgaccaggagcgatccatttccaagatcgagaggatggattgtcacacggatggatatgaggatgagatggaagatggtgagcttgagagtgattcaagttcttacagagttctatccagtaagacacttgcagatgttgtggaagcactaattggggtatattatgtagaaggtggtaagaatgctgccaaccaccttatgaaatggatgggcattcaaatagagtttgatcctgatacaatggattgtacaagaaagccatttaatgttccagatagcatacttaggagtgttgattttgatgctttagaaggtgctttaaatatgaagtttaaagacaggggactgctggtagaatccataactcatgcctccaggccatcttcaggagtatcctgttatcagcggttggagtttgttggtgatgctgtcttggatcatcttattaccaggcacttgtttttcacttacacaaatctgccaccgggacgtctgactgaccttcgggctgctgctgtaaacaatgaaaattttgcgcgtgttgcagttaaacacaaccttcatgtgcatctccggcatggatccagtgccctagaaaaacagattaaagaatttgttaaggaagttcaagatgaattgtcaaagccaggttttaattcctttggtctgggagactgcaaagcccccaaggtccttggtgacattcttgaatctattgcaggtgccatttttcttgacagtgggagggatactactgttgtttggaaggtttttcagcctcttttgcatcctatggttacccctgagactctgccaatgcatcctgtgcgtgagctgcaagagcggtgccagcagcaagctgaaggcttagagtataaagcaagtcgtattggcaatttggccactgttgaagtattcattgatggggtgcaagtaggggctgctcaaaatccacagaaaaaaatggcgcagaaattagctgcaagaaatgcccttgctgctttgaaagagaaggaggtgggaaaaactcaggaaaagaatgatgacaatgggaagaagaatggaaaccaaacatttactaggcaaacattaaatgatatctgtttacgaagaaattggcctatgccattttacaggtgtgtgaatgagggtggtccagcacatgcaaagaggtttacatttgcagtgcgtgtcaatactactgataaaggatggacagatgaatgtgttggagagccaatgccaagtgtcaagaaggccaaagactcagctgctgttcttctcttagaactactgaacaaattatactcgtga

SEQ ID NO: 67 - DNA, 5652 bp, Oryza sativa
  misc_feature 1..5652: note = ORF of protein Dcl1
  source 1..5652: mol_type = unassigned DNA
atggcgggaggaggaggagttggaggaggcgctggcgagcacgcggcggcggcgtactggtacgacgcgtgcgaggacggcgcgtcgctgctgtgcggcatcgacttcgccgcgtccgcggacttcgaccccggcctcatacccgcgatggacaccggcgccgacgacggcttcgtcgccgagatcgacaggatactcgagagcatcaacgcggagagctcccctgcgcccccacccccgccgccgccgccgctacctgagcccgtgcctgtggctccgccggagctaccgattcaggaaaagcaattacaggtggcctctgcgcccgtggccaacaatgccgtggcggtggtgggcgttgtgcagagaagtaaaggcgtggtggcgaggaaggagccgaggagggagtcgcatggctgtgctgcaaatggcggcggcggtggggagtggagggacgggaagcgcccgcgcctcgcctccggtggtgtcgggggaccgcgtcaggagtggcggcggaggccgatgctgccaccaccgccctcgcgtgggtgggatgacaggcgtggaaggcgcgatttcgacagggtccgcaagcacgagcaccatcggcgcgaggcccggggtttctgggagcgtgaccgcggcggcaagatggtgttccgctcaggcacctgggagcaggagtctgaccgcgaggcaaagcgtgccaggacgcaagatggaggctccatggagaagaaggcagaggcagataggatgggggctgcgcagcgggagaagcctgtcgcggaggagcgtgcaaggcaataccagctagaggtgcttgagcaagctaagagcaggaacacaatagccttcctggagactggcgcagggaagactctcatcgccgtgcttctcatcaaaagtgtgtgcgataaaatgctcaaggagaacaagaagatgcttgctgtcttcttggttcccaaggtgcctcttgtgtatcaggtactagttatgacagcccaaattttgttaaacatcctcaggcacagtattattaaaatggatgcgatacatcttttgattttagatgagtgccatcatgcagtgaaaaaacatccatattctctagtgatgtcagaattttatcacacgactccgaaggagaagaggccagcagttttcggtatgacggcttctcctgtcaatctaaaaggagtgacgagtcaagaggactgtgccatcaagataagaaatcttgagagcaaactagattcagtagtttgtaccatcaaagatcgtaaagagcttgagaagcatgtccccatgcctttggaagttgttgtccaatatgacaaagcagccactctttggtccttgcatgagcaaatcaaacaaatggaaagcacggttgaagaagctgcactctctagctctaagagaactaaatggcagttcatgggggctagggatgctgggtctagagatgagctgcgtcttgtctatggtgtttcagagcgtacagagagtgatggtgcggctaatttaatccagaagttgagagctataaattatgctttaggtgaacttggacaatggtgtgcatataaggttgcacagtcctttttgactgcattacaaaatgatgaaagggcaaactatcaggttgatgtcaagtttcaagagtcttatttaaaaaaggttgttgatctgttacactgccagttgactgaaggggctgcaatgaaaagtgagaccagtgatgttgaaatgcaaaatactgaaaaacacaacacaaatgaccttgaggagggggagctgcctgacagtcatggtgagcatgtagatgaagttatcggtgctgccgtagcagatggtaaagttactcctagggtgcaggctttgatcaaaatacttctcaaatatcagcatactgaggattttcgtgccataatctttgtggagagagttgtgacagcattagttcttcctaaggtattggcagaacttccatctctaagttttattcgatgtgcaagcttgataggccataacaataatcaagagatgcgcgcgtgccagatgcaggatacaatttcaaaatttcgtgatggccgtgttacacttttagtcgcaacaagcgttgcagaggaagggcttgatattcgacagtgcaatgttgtcattcgatttgaccttgcaaagaccgtcttagcctacatacagtcacgtggtcgtgcaagaaagcctggatctgattatatattaatgcttgaaaggggcaatatatcacatgaaacttttttgaggaatgctcggaatagtgaggagacgttgagaaaggaagctatggaaagaaccgatcttagtcatttggatggtacttctgtgttgagtcctgttgatacatcacctggttcaatgtatcaggttgaatcaacaggtgctgttgtcagcttgaactcagcagttggactcatacatttttactgttcacagttgccaagtgacagatactcaattcttcatccggaattcattatgcaaaagtatgagaagccaggtggttcagtggaatattcttgcaagcttcagctcccttgtaatgccccgttcgaaaaacttgagggtcctatatgcagttcaatacgactggcacagcaggctgtttgtttggctgcttgtaagaagctgcatgagatgggtgccttcacagatacgctcttacctgacagaggaagcggggaaggggaaaaaactgagcagaatgatgaaggtgaaccacttcctggaacagctcgccatagagagttttatcctgaaggtgttgctgatattctacggggagaatggattttatctggcagagatggctaccaaaactctcaatttattaagttatatatgtattctgtgaactgtgtaaacgttgggacttctaaggaccctttcgttacacaactttcaaattttgcaattatttttggcaatgagctggatgcagaggttttatcgacgacgatggatctctttgttgcaaggacaatgataacaaaggcatctctcgtattccgtggacgaattgaaattacagaaagtcagctggtgttgctgaagagctttcatgttaggctcatgagcatagtacttgatgttgatgttgatccctcaactacaccttgggatcccgcaaaagcatacctctttgtccctgtaggagctgagaagtgtacagatcctttaagagagattgattggactttggttaacaacattgttaacactgatgcctggaacaatccacttcagagggcacgcccagatgtctatctaggcaccaatgaacgaacacttggtggggataggagggaatacgggtttgggaaattgcgccatgggactgcttttggacaaaaggcccatcctacttatggtattaggggagctattgctgaatttgacattgtgaaagcatctggattagttcctgctcgtgatagaggacattttagtgattatcagaaccaaggcaagttgttcatggcagattcatgttggaatgctaaagatcttgctggaatggttgtcactgctgcccactcagggaagaggttttatgtggactgcatttgctataacatgaatgcagaaaattcattccctaggaaagaaggctacctggggccattggagtatagttcatatgctgattactacaagcagaagtacggtgtggagttaatctataggaaacagcctcttatacgggcccgtggtgtttcatattgcaagaatcttctttctcctagatttgagcattctgacgctagagaaggtgacttctctgagaaccttgacaaaacatactacgtgtatttgccacctgagttgtgccttgtgcacccacttcctggatcactagttcgtggagctcagaggttgccctcaataatgagaagggtggagagcatgcttcttgcagttcaattgaaggacataattgattaccctgttcctgcaactaagatactagaagccttgactgctgcatcatgccaggagacattatgctatgaaagagcagagctgctgggtgatgcatacctgaaatgggtcgtgagtagattcctcttcctaaaatatcctcaaaagcatgagggacagcttacaaggatgcggcagcagatggttagtaacatggtcctgtaccaatatgctctgaataaaaccctccaatcatatattcaagcagaccggtttgctccatcaagatgggcagctccaggagtgctacctgtattcgatgaagagtctagagagtatgaaccttccatctttgacgaggaatcaactggttgcgagttacagaaggaatcctatgatgactatgcagataacatgcaagaagatggtgaaattgaaggagactctagctgctatcgggttctgtcgagcaaaactttagcggatgttgttgaggcacttattggggtgtattatgtagcaggagggaaaatcgctgcaaaccacctgatgaaatggattggaattcatgcagagttggatcctgaagagatcccacctccaaagccatatgacattcctgagagcataatgagaagcatcaactttgacacgttgaaaggtgtcttgggtatcgaattccagaataaaggtttacttgttgaggccataacacatgcatcgagaccatcttctggtgtttcctgttaccagcgtttggaatttgttggtgatgctgtgttggatcatctcataaccaggcatttgttttttacatatactgacctacctcctggccgcttgactgatttgagagctgcagctgttaataatgagaattttgcaagggttgcagttaagcataagctgcatgtacatcttcgccatggatcatctgcattagaaacacagattcgagagtttgtgaaagatgtccaagaggagcttttgaaaccaggtttcaattcttttgggcttggggattgtaaggctcctaaagttcttggtgacattgttgagtcgattgctggcgcgatattcctggatagtggatatgatacctcagttgtctggaaggtttttcagccactccttcatccaatggtgacgccagagacccttccgatgcatccagttagggagctccaagaacgctgccaacagcaggctgaaggcctggaatacaaagcatcccgagcaggcaacatagctactgttgaagtatttgttgatggtgttcagatcggtgtagctcagaaccctcagaagaagatggcacagaagttggcagcgaggaatgcacttgtggtcctgaaggagaaggaaaccgcaacaaagaaagaagatgaaagggatggcgagaagaaaaatggtgctcagatgttcaccaggcagaccctgaacgacatctgcttgagaaggcagtggccaatgcctcagtacaggtgcgtaaacgagggtggccctgcccatgctaaaagattcgtctattcggtgagggtgaacacttctgatcgtggatggacggatgaatgcattggagagccaatgcctagtgtgaagaaggctaaagactctgctgcagttcttctcctcgagcttctgaatagagatttccctgacaagcctgacggtaagcagccgtag

SEQ ID NO: 68 - DNA, 5667 bp, Solanum lycopersicum
  misc_feature 1..5667: note = ORF of protein Dcl1
  source 1..5667: mol_type = unassigned DNA
atggatggtgatggtagtggagtttctgagaaaccttcttattggattgatgcttatgaggacatctcatgtgatgatctcatccaagattttgttgggtccaacactgtacccgttgcaccattgaatcaattgggtccggatgagactcttgatccttgtttctttggtggtattgatgggattctcgagaaattaaagaatggtgatggttctgtacattctgtggatggaaacaacagtaatgtacacactgaagctttagctgcacctggaatttgcaaggacaacgggccacaagttaaaaaggatgtggaccacaacaaaggtgatttggggaagaagtctttacaagaaggaaatggattttccaggcataaggaacggggttgtagtgataaagaagagaaaaatggaaaaagggcgcggcttggtgatgatagctaccagaggagaggctgtgatagacctttggctagaaagagacttcgcgagaatgatgagatcaatagagtagggagggatcaaagaaaaagaagagaatatcatggtggtcgtggtggaagagataggaactggagggaagggagtggcttttgggaatgggacaaagaaaagaaagagatgatctatcgtgtgggttcatgggaagctgatagaaatcgagaggggaagttaccaactgagagaagccgtgaaccttctggagctattgagaagaaagatgacaaattaaaggaacaagctcccaaagaggaagcacgcaagtatcagttggatgtacttgagcatgccagaaagagcaatacaatagcttttcttgaaacaggagcaggaaaaacgcttattgccatcctcctcatgaagagtctatgtagtgacttgcaaaagaagaacaagaaaatgctggcagtttttctggttcctaaggttccacttgtttatcagcaagcagaagttattcgtgagcaaactggctatcaagttggccattattgtggtgaaatgggccaagatttttgggatgcacgaaggtggcagcgcgagttcgaaactaaacaggtcctagttatgactgctcagattctcttgaacatattgaggcacagtataataaaaatggaagcaattaatctccttattatggatgaatgccaccatgctgtgaagaagcatccatattctttagtaatgtctgagttctatcatacaacacaaaaggagaaaaggccatctgtttttggcatgactgcttcacctgttaacttgaagggtgtttccagccaggttgactgtgcaataaagattcgtaatttggagactaaattagattctgtcgtctttaccataaaagaccgcaaggagctggaaaaacacgtgccaatgccgtcagaagtagtagtggagtatgataaagctgctagtctgtggtcttttcatgaacaaataaaaaaaatggaatcagaggttgaagaagctgctctgttaagctccagaagaagtaaatggcagtttatgggggcttgtgatgctggagctagaggagagctgcgccaagtctatggtgtttctgaaagaactgaaagtgatggagctgctaatctaattcaaaagttgagggctatcaactatgctcttggtgaacttggacagtggtgtgcctataaggtagcacattcttttttgacagctctacaaaatgatgaaagggcaagttaccagcttgatgtcaagttccaagaatcctacctggataaggttgtctctctcttgcaatgtcagttatcagagggagctgtagcacaaagtaacttgaatgcagagacgcataaaggtgacaatccaaatagtgataggcctgatgagatggaggagggcgagcttcttgaaagtcatgttgtatctgttggagagcatgtagatgtgattctaggagctgcagtggctgatgggaaagtgacacctaaagttcaatcattaattaagattcttctcaagtatcagcatactgaagattttcgtgctataatatttgttgaacgcgttgtgactgcattggttcttccaaaggtttttgaggagctcccttccctaagttttatcaattcatcaagtttaatcgggcataacaatagtcaagaaatgcgaacaggtcaaatgcaggacacaatagccaaatttcgagatggtcgtattaatttgctagtagctactagtgtagctgaagaaggacttgacatccggcaatgcaatgttgtcattcggtttgatctggccaaaactattttggcgtatatacagtctaggggccgtgcccgtaagcccggatcagattatattttgatggttgagagagataattcatcacatgaagcatttctgaggaatgccagaaacagtgaagaaacgctgcggaaagaagcaattgagcggactgatattagtcatctcaaagatgcctccaagttgatctctgcggaggcaccaacagattcagtttatcaggtggagtcaactggagctgttgtgagcttaaattctgctgttgggctgatccacttttattgctcccaattaccaagtgacaggtactcgattctccgtccggagtttataatggagagtcacgagaagtcaggttgccctactgaatattcatgcaggcttcaactgccctgcaatgcacctttcgagacacttgatggccctgtatgcagctcaatgcgcctcgcccagcaggctgcttgcttggatgcttgcaagaagctccaccagatgggggcatttacggacatgctcttgcctgacaagggaagtggggtggagtcggagaaagttgaacaggatgaagaaggagatccaattcctggaacttccaggcacagggaattttatcctgaaggtgtagctgatattctgaggggtgattggattttgtcgggaaaggatcctcttgtcagctcaaagttcattcatctttatatgtatgctatcaaatgtgtaaatattggcccctcgaaggacccattcttaactgatgtgtcagagtttgcaatactatttggcaacgagctggatgcagaggtattatcgatgtcaatggatttatttattgctcgaactgtagtaacaaaggcgactcttgtcttcagagggccaatagatgttacagagtttcagttggcgtccctcaagagttttcatgtaagaatgatgagcattgttttggatgttgatgttgagccgtccaccactccatgggaccctgcaaaggcatatctatttgcccctgttagtggtgatgaatctggagatcctataaaagatatcaactgggatcatattaaaaaaattaccgaaactggtgtatggagcaatcctcttcagagagctcgcccagatgtatatcttggtacaaatgagcgttctcttggtggagaccgaagagaatatggctttgcgaaactgcgacatggcatggccatcggactgaagtctcatcctacatatggtgttagaggtgctatagcacactatgatctggtccaagcatctggtttggtccctaaccgaagtagccttgatgatgttgaagttgatttgaataaagacaagataatgatggctgattgttctcttagagccgaagatattgtaggaagaattgtcactgcagctcactctggaaagagattttatgttgattgcattcgtagtgatatgactgcagagaactcatttccaaggaaagaaggttacctaggtcctcttgagtacagcagttatgctgcatattataagcaaaagtatggagttgatttggtctataaaaagcaacctctaataagagggcgtggtgtttcatattgcaaaaaccttctgtcaccacgatttgaacattcagaagaacatgagggtgaactcgaagaggcaactgacaaaacgtattatgttttcctccctcctgagctctgtgttttgcatccacttcctggatcccttgttcggggtgctcaaagattgccctcgatcatgaggaggattgagagcatgctgcttgctgttcagcttaaggaaatgattggttaccctgtaccagcactgaagatcttggaagcattgaccgctgcttcttgtcaggagactttctgctatgaaagagcagaacttcttggagatgcatatctgaaatgggttgttagtcggtatctttttcttaagtatccccagaaacatgaaggccaactcaccaggatgagacagcaaatggtaagcaacatggtactgtatcaaaatgccttgagtaagggaatccagtcgtatatccaagcagatcgattttctccatcaagatgggccgctcctggggtgttgcctgtctatgacgaggacctcaatgaggatgaaacatcgatatttgatcatgaaacagcagaaaatggtactgtagcagcaaaggctctagctggtgatgagtttgaagatgaggaaactgaagaaggagtatattacgttgagggtggaaagtatgctgcaaaccacttcatgaaatggattggagttgaggttgatttcgatttcaatttcaaagagacagaatactctattaggtcttgtagcattcctgagaatgtactcaagagtgttgagtttgatgcattacaaggtgctttgaacatcagcttcaatgataaaggcctactgctagaagcaattacccatgcttcacgcccatcttctggggtctcctgttatcaaagactggagtttgtgggtgatgcagtattagatcatctcatcacgaggcacttgttctttacatacacagatcttcccccaggccgattgactgacttgcgagctgcggctgtcaacaatgaaaattttgcacgtgttgctgttaagcacagtcttcatttacaccttcgtcatggatccagtgctctagaaaaacagattcgtgactttgtgaatgaggttaaaaatgaactatcaaaaccaggtttcaactcctttggtttgggagattgcaaagctcctaaagttcttggtgatattgttgaatcaattgctggggctatttttctagacagtggatgtaatactaaatctgtatgggaggtttttcaaccgctattgcatccaatggttacaccagaaacactgccgatgcatccagttcgagaattgcaagagcgttgtcaacaacaagctcaaggcttagagtacaaagcaactcgaagtgggaatatggctacagttgaagtatatgtggatggtgttcaggttggaatggcgcagaatcctcagaagaaaatggcacagaagttggctgccaggaatgcccttgttgtgttgaaagagagggaggaagctgaagctaagaaggctgatgatacaaagaaaaagaagaatggcaacccgagttttactaggcagacactaaatgatatatgcttgcgcaaaaattggcctatgccactgtatcgatgtgtgcacgagggtggtccagcacatgcaaagaggtttacatatggagtacgcgtaaatatttcagacaaaggatttacagatgaatgtattggggaacctatgccgagtgtaaagaaagccaaggattccgcagcagctcttctattggaactcttaaatagacggtactcgtaa

SEQ ID NO: 69 - DNA, 3924 bp, Zea mays
  misc_feature 1..3924: note = ORF of protein Dcl1
  source 1..3924: mol_type = unassigned DNA
atgaaaagtgacagtaatgatgttgaaatgcataattctgaaaaccccaagccaaatgaacttgaggagggagagctacctgacagtcatggtgaacatgtagatgaagtcattggtgccgcagtggccgatggaaaagttaccccaagggtgcaagctttgataaaaatacttctcaagtatcagcacactgaggattttcgtgctatcatatttgtagagcgagtggttacagcattagtcctccctaaggtctttgcagaacttccatcgttgggctttatacggtgtgcaagcttgataggtcataacaataaccaggagatgcggtcgtgccaaatgcaggatacaatcacaaaatttcgtgatggccgtgttacactgttagttgcaactagtgttgcagaggaaggacttgatattaggcagtgcaatgttgtcattcgctttgaccttgcaaagactgttttggcctacattcagtctcggggtcgtgctagaaagcctggatctgactacatattgatgcttgaaagaggaaatatgtctcatgaagctttcttgagaaatgctcggaatagtgaagagacattgagaaaagaagctattgagagaactgatctcagtcatctcgatggcacttctatgctaagccctgttgatatatcacctgattctatgtatcaggttgaatcaacaggtgccgttgtcagtctgaactctgcagttggactcgtacatttttactgctcacagctgccgagtgacagatactctattcttcgaccggaatttattatgcaaaagcatgagaaaccagggggttcaacagaatattcttgcaaacttcaactcccttgtaatgccccattcgagaaacttgaaggtcctatatgcagttcaatacgcctggcccaacaagctgtttgtttagctgcttgtaagaagctacatgagatgggtgccttcacagatatgcttttacctgacagaggaagtggggaaggagaaaagactgaacaaaatgatgaaggtgatccactgcctggaacagcacgccatagagagttctttcctgaaggggttgctgaaattctacatggagaatggattttatctggaagagatggttaccaaagttcacagtttattaagttatatttgtattctgtgaactgtgtaaacattgggacttcaaaggaccctttcgttacacaactttcaaattttgctcttatttttggcaatgagctggatgcagaggttttatcgacgacgatggatctctttgttgctaggacaataataacaaaggcatctctcgtattccgtggaccaattgaaattacagaaagtcagctgatcctgcttaagagctttcatgttaggctcatgagcattgtacttgatgttgatgttgatccctcaacaactccttgggatccagcaaaagcatacctctttgttcctgtgggagctgagaaatgtatggatcttttaagagagatagattggactctagttaatagcattgtgaattctgatgcctggaacaaccctcttcagagggcacgcccagatgtctatctaggcacgaacgaaaggacacttggaggggataggagggaatatgggttcgggaagttgcgccatggaactgcatttggacagaaagcccaccctacgtatggtatcagaggagccatcgctgaattcgatgttgtcaaagcgtctggattagttcctggtcgtggatggggacattttaatgattatcaaaataaaggcaagttgttcatggcagattcatgttgggatgccaaagatcttgctggaatggttgtaactgccgcccactcaggaaaaaggttctatgtggacagtatttgctataatatgaatgcagaaaattctttccctaggaaagagggctatctgggtcccttggaatacagctcgtttgctgattactacaagcagaagtatggtgtggagttagtttataagaaacaacctcttatacgggcacgcggtgtttcatactgcaaaaatctactttctcctagatttgagcattctgaagctagcaatggagagttctcagagaatcttgacaagacatactatgtatatttaccacccgaattgtgccttgtgcatcctctccccggatcacttgttcgtggagctcagaggttaccctcaataatgagaagggtggagagcatgcttcttgcaattcaactgaaggacataattgggtatccagttcctgcaaataagatattagaagccttgactgctgcgtcatgccaggagacattctgctatgaaagagcagagctgctgggtgatgcatacttgaaatgggttgtgagtagattccttttcctaaaatatcctcaaaagcatgagggacagctcacaaggatgcggcagcagatggttagcaacatgatcctgtatcaatacgccctgaataaaaacctccaatcttatatccaagcagatcgatttgctccatcaagatgggcagctccaggagtgttacctgtatttgatgaagaaacgagagattctgaaccatccatctttgatgaggaatttactcctagcagtgagttacaaaagaacttgtatgatgactatgatggcgatagcatgcaagaagacggtgaaattgagggagattctagctgctatcgtgttctatcaagcaaaacattagcagatgttgtggaagcacttattggtgtctattatgtagctggagggaaaattgctacaaaccatctgatgaaatggatcggtattaatgcagagttggatcctcaagagatcccatcttcgaagccatataacatacctgagagcataattaaaagcatcaattttgacacattagagggtgcattgggtatcaagtttcagaataaaggcttacttgttgaagctataacacatgcatcaagaccatcatcaggtgtttcctgttaccagcgcctggaatttgttggtgatgctgtattggaccatctcattacgaagcacttattttttacttatacggacctacctccaggccgcttgactgacttgagagctgcagctgttaataatgagaactttgcaagggttgcagttaaacataagctacatgtacatcttcgccatggatcatctgcattggagacacagattcgagagtttgtgaaggatgtccaggaggagctttcgaaaccaggtttcaattcttttggccttggggattgtaaggctccaaaggttctcggagacatttttgagtctattgctggcgcaatattccttgatagtgggtgtgctacctccgtagtctggaaggtttttcagcctctgcttgatccaatggtgacgccggacacccttccgatgcaccctgtaagggagctccaagagcgctgccagcagcaagccgaaggattagaatacaaagcatcccgtacaggcaatgtagctacagttgaagtcttggttgatggcatacagattggtgtagctcagaacccacagaagaagatggcacaaaagttggcggctagaaatgcacttgttgtcttgaaagagaaggaaaccgcagcaaagaaagacactgaaaaggatggcgagaagaaaaacggttctcagttcactaggcagactctgaacgacatctgcttgagaaggcaatggccgatgccacagtacaggtgtataaacgagggtggccctgcccatgccaaaagatttgtttatgcggtaagggtaaatacatctgacagtggatggactgacgagtgcattggagagccaatgccgagtgtgaaaaaggccaaggactctgcagctgttcttctgctcgagctgctgaatcgaaattaccgtggcaagcctgatggaaaataa

SEQ ID NO: 70 - DNA, 5601 bp, Brassica rapa
  misc_feature 1..5601: note = ORF of protein Dcl1
  source 1..5601: mol_type = unassigned DNA
atggtaatggaggatgagcgtagtaataaggatgtcaacaacaagccatcttactggctagatgcttgcgaggacatctcctgcgatctcatcgatgacctcgtctccgatttcgatccttcctctgtcgccgtcgctgagtccatcgtcaacaacgacttcttcggtgggatcgatcacattctagacaccatcaagaacggtggaggcttgccaaacaacgaggctcctcagttgaatcctggagatgttgttactaaggagaatggtttgcaaaagactggtcttaaaagagacgatgcatcgaaagaggaaggagacaagaacaggaagagagctagagtttgtagttaccagagcgagaagaataggaaacggactcgaagctacgaggaggctggtggtcataacaagagaagagatggttacaattacagaagagacggtagagataatagagaaggtagaggctattgggagagggataaagtcggttcgaacgagcttgtttatcggtctggtacttgggaagctgatcacgaaagagatgttaagaaagagagccgtagaagccaccgagaagaggcagaggaggataataagaagagtaagcctgaagaacagcgtaaagagaaagttgtggaagagcaagcgaggcggtaccagttggacgtgcttgaacaagctaaagcgaagaacacgatcgctttccttgagaccggtgcggggaagacacttatagccattctcttgatcaaaagtgtgcataaggatctaatgagccagaacagaaaaatgctctctgtcttcttggttcctaaagtgcctttggtttatcagcaagcggaggtgatacgtaaccaaacttgttttcaagttgggcactactgtggtgagatggggcaagacttctgggatgcgagaaggtggcagcgagagttcgagtctaagcaggttcttgtaatgacggcacagattctgttgaacatactgaggcacagcattatcagaatggaatctattaatctactgatcctcgacgagtgtcaccacgctgtcaagaaacatccttactcactggtgatgtcagagttctaccacacaacgtctaaggataaaagacctgccatctttggcatgactgcttctcctgttaatctaaaaggtgtttcaagccaggtagattgtgctataaaaatacgcaatctcgagacaaagttggattctacggtttgtactataaaagatcggaaggaactagagaaacatgttcccatgccttcagagattgtggtggagtatgacaaagctgctactatgtggtcttttcatgagaagataaagcaaatgattgcagctgttgaggaagcggcgaaggcgagttcgaggaaaagcaagtggcagtttatgggggctagggatgctggagctaaggaggagctgaaacaggtttatggcgtctctgagagaacagagagcgatggagctgctaatttgattcataagcttagagctattaattatactcttgctgagttgggtcaatggtgtgcttacaaggtggcacaatcgttcttgactgcgttgcaaagtgacgagagggtgaatttccaagtggatgtgaagtttcaagaatcttatctcagcgaggtggtgtcgctcttgcaatgtgagcttgtggaaggagctgctgctgagaaagcagccacggaatgtagcaaaccagagaatggtaatgcaaatgatgagattgaggaaggagagctccctgatgatcatgtggtctctggaggggagcacgtcgatgaagtaataggcgccgcagttgctgatgggaaagttactccaaaggttcagtcgctgatcaaactactcctcaagtatcagcacacatctgattttcgtgctattgtgtttgttgagagagtggttgctgctttggttcttccaaaggtcttcgcggagctgccttctcttggttttataaggtgtgccagcatgattggtcacaataacagccaggaaatgaaatcatctcaaatgcaggatactatttccaaattccgagatggtcaagtgacactgttagtggccacaagcgttgctgaggaaggtcttgatatcaggcaatgtaacgttgttatgcgttttgaccttgcaaagacggtgctggcatacattcagtctcgtggtagggcaagaaagcctggttcagactacatactcatggttgagagggaaaatgtatctcacgcagcctttttaaggaatgctaggaacagtgaggagacactgcggaaagaagcaatagaaaggactgatctgagtcacctcaaagatacatcgaggctaatctccattgacgctgtccctggtacagtttataaggtggagacaactggtgccatggtcagcctgaactccgcggttggtctcatacatttctactgctctcagcttcccggtgacaggtatgcgattcttcgacctgagttcagcatggagaaacatgaaaagcctgggggacacaccgagtattcatgcaggcttcagctcccttgcaacgctccatttgaaatactcgagggtcctctttgtagttcaatgcgccttgcacaacaggctgtatgtctagctggttgcaagaaactgcacgagatgggcgcatttacggatatgctgttaccggacaagggaagtggtcaagacgctgagaaggctgaccaggatgaagaaggtgaacccgttcctggaactgctagacacagagagttctatccagaaggtgtagcagatgtacttaagggagattggattttatcaggaaaggaagattgtgagagctcaaagctattccatctatacatgtacagtgttagatgtgaagattctgggtcttcaaaagacccattcttaaccgaagtttcagagttcgcggttctttttggcaatgagctggatgcagaggtattatcgatgtctatggatctttatgttgctcgagccatgatcaccaaagcatctcttgttttcaagggatcactggatattacagaaagccagctatcatctattaaaaagtttcatgtgaggttgatgagtatagtcttagatgttgatgtcgagccctccacaacaccatgggatcccgcaaaggcctacttgttcgtccccgtgactgacaacacgaaagggatcaactgggaactaattgaaaagataactgagaccaccgtgtgggacaacccgcttcagagagctcgtcctgatgtgtaccttgggactaacgagagaactcttggtggggacaggagggagtatgggtttggtaaacttcgtaacaacattggattggggcagcagaaatctcacccgacttatggtattagaggagcggttgcatcttttgatgtcgtgagagcttctggattgttacctgtgagagaggaagtaagaagtgatgtatcacaagggaaactgatgatggcagatgggtgcatggttgcagataatctaatagggaaaatagtgacggccgcacattcagggaagcggttttatgtggactctatatgctacgatatgagtgcagaaacatcattcccgaggaaagaggggtatcttggtcctttggaatacaacacttacgctgactactataagcaaaagtatggagttgatttaaactgtaaacaacagcctttgatcaaaggacgaggtgtttcctattgcaagaaccttctttctcctcggtttgaacagtcaggtgaatctgagacgatacttgataaaacatattacgtgtttcttccacctgagctgtgcgttgtgcatccgctttcgggttcacttgtccgaggggctcagaggttgccctctataatgagaagagttgagagcatgttgctcgctgttcagctgaaaaacttgattagttatcccattccaacttcaaagattctggaagcgttgacagcagcctcgtgccaggagacgttctgctacgagagagcagaactcttaggagatgcgtatctgaaatggatcgttagtcgttttctgtttctcaagtacccgcagaagcacgagggtcagcttacgaggatgagacagcagatggtcagcaacatggttctttaccagtacgctcttctcaaagggctccagtcatacattcaagcggatcgtttcgcaccttctaggtggtctgctcccggcgtgcctcccgtttacgacgaggacaccaaagacggaggaggaggatcttccttcttcgacgaagagcagaaacaagcctcctccgaggacgtgtttgaagatggggagatggaggatggcgagctagagggtgacttgagctcttaccgagtcctatctagcaagacgttggctgatgttgttgaagctttgatcggtgtttactacgtggaaggaggtaaagctgctgcgaaccatttgatgaaatggattgggatccacgtggaggatgatcccgaagaaaccgaaggggccgtgaaaccggttagtgttccggagagcgtgctcaagagcatagactttgttggtttagagagagctttgaagtacgagtttacggagaaaggtcttcttgtggaagctatcacgcatgcttcgagaccgtcttcgggtgtttcgtgttaccagaggttggagtttgttggggacgcggtgttggatcatcttattacaagacacttgtttttcacgtacacgagccttcctcctggtcggttaactgatcttcgagctgctgcggttaataatgagaatttcgctcgtgttgcggttaaacataagctccacttgtaccttcgtcacggctctagcgctctcgagaaacagattagagagtttgtgaaggaagtcctaaccgagtcttctaaaccggggtttaactcgttcggtttgggagattgtaaagcgccaaaggttcttggagatatagttgaatccattgcaggtgctatttttcttgatagtggaagagacacaagtgctgcttggaaggtttttcaacctttgctacaaccaatggtgacaccagagacacttccgatgcatccggtgcgagagctgcaagagcgttgccagcaacaagcagaagggttagagtacaaagcgagccggagtggtaacacagcgactgtggaagttttcatcgacggtgttcaagtgggagtggcgcagaatcctcagaagaaaatggctcagaagctagcggcaaggaacgcgctggcagctttgaaagagaaagagatagctgagtcaaaagagaaggcggcaaacgggaatgcaggagacgagagtgagaatgggaacaagaagaatgggaatcagacgtttacgagacagacgttgaatgatatttgcttgaggaagaattggccaatgccttcttacagatgtgtgaaggaaggaggaccggcgcatgcaaagaggtttacatttggggtaagagtgaatacaagcgatagaggatggactgatgaatgtattggagagcccatgcctagtgttaagaaggctaaagattctgctgcttctcttcttcttgagcttcttaataaaacttattcttga

SEQ ID NO: 71 - DNA, 5994 bp, Hordeum vulgare
  misc_feature 1..5994: note = ORF of protein Dcl1
  source 1..5994: mol_type = unassigned DNA
atggcgggaggaggaggaggaggtggcgcgggcgagcacccgggggcggtgtcctactggtacgacgcctgcgaggacgacgaggacgcgtcgctgctgtgcggcatcgacttcgcctcgtccgccgacttcgacccggggctcatgcccttgcccgccatggactgcggcatcgacttcgccgcctccgcggatttcaaccccggcttcatgcccgccatggactgcggcggcgtagacgacggattcgtcgccgagatcgaccggattctcgagagcatcgacgccgagacagcccccgcgccacagcccccgcctgtggctcccccgcaaccgccggcggagcgggtgcaggaggctgctgctattgcggctgtggccaacaatgccgtggcggtggtgtgctcggagcagaggagccaggccggcgggcccaggaacgaggcgagaagcgtcgaggcgaggaaggagacgagaagcgtcgggccgaggaaggagacgacaagcgccgagccgaggaaggagacgagaagggagacgcaaggcggttcggcgaacggcggtcgcggcggggagtggagagacgggaagcgtcctcggctcgcccctggggctgacgggggcccacgccctgattcccggcggcgcccgatgctgccgccgccctcgcgtggctgggatgaacggcgcgggaggcgcgacttcgtcgacaggcctcgcaagcgcgaccgcgaccgcgacagtaaccctgcccaccggcgcgaggctcgaggattctgggagcgggaccgcggcggcaagatggtgttccgccctggcacctgggagcaggagtctgagcgcgagggaaagcgtgccaggacacagaacggaggctccatggagaagaatgcagacgcagataaggcggcttccgcccacaaggagaagcctctcgccgaggagcaggccagacattaccagttggaggtgctcgagcaagcaaagagcaggaacacaatagccttcctcgagaccggcgcagggaagaccctcattgctgtgctgctcatcaaaagcatatgtgacaaaatgatgaaggagaacaggaaaatacttgccgtgttcttggttcccaaggtgcctcttgtgtatcagcaagctgaggtgatacgcgagaggactggctatcgggttgggcactactgcggagagatgggtcaggatttctgggatgctaggaagtggcagcgagagtttgattccaaacaggtactagttatgacagctcaaattttgttaaacatcctcaggcacagtattattaaaatggacgccatacatcttttaattttagacgagtgccatcatgcagtgaaaaagcatccatattctctagtgatgtcagaattttatcacacaactccgaaggataagagaccagttgtttttggtatgacagcttctcctgtcaacctaaagggagtcactagccaagaagattgtgccatcaagataagaaatcttgagagtaaactggattccgttgtttgtactatcaaggatcggaaggagcttgagaaacatgttcccatgccttctgaagttgttgtccaatatgacaaagcagccaccctttgttccctgcacgagcagatcaaacaaatggaagctgctgttgaagaagctgcactctgtagctctaagagaacaaagtggcagtttatgggagccagggatgctgggtctagagatgagctgcgccttgtctatggtgtttctgagcgtacagaaagtgatggtgcagctaatttaatccagaagttgagagctataaattatgctttaggtgaactaggacaatggtgtgcttataaggttgcacagtccttcttgacagtattgcagaatgatgaaagggcaaactatcaggttgatgtaaagtttcaagagtcgcatttaaagaaggttgttgatttattgcactgccagttgactgagggagctgcagtgaaaagcgagacgaatgatgttgagatgcataacactgaaaatcacaacccaagtgaacttgaagatggggagctgcctgatagccatgctgtttctgtaggtgaacatgtagatgaggttatcggagctgccgtggcagatgggaaagttacccctagggtgcaggctttaatcaaaatccttctcaagtatcagcatactgaggatttccgtgctatcatctttgtggagcgagttgtcacggcattagttcttcctaaggtgcttgcagagcttccatctttaggctttatccgatgtgcaagcttgataggtcataacaacaatcaagagatgcgcacaagccaaatgcaggatacaattgcaaaatttcgtgatggtcgtgttacactgttagttgcgactagtgtcgctgaggaaggacttgatattcgacagtgcaatgttgtcattcgctttgaccttgcaaagacggttttggcctacgtacagtctcgtggtcgtgcaagaaagcctggatctgactacatattgatggttgaaaggggaaatttggcacatgaaactttcttgaggaatgctcgaaatagtgaggagacgttgagaaaagaggctatagaaagaactgatctcagtcatcttgatggtactctgttaaattccattcatataccacctgattcgatgtatcaggtcgaatcaacaggtgctgttgtcagcttgaattctgcagttgggctcgtacatttgtactgttcacagcttccaagtgacaggtactcaattcttcgtccagaatttattatgcaaaaacatgagaagccagggggttccatggaatattcttgcaaacttcaacttccctgtaatgctccatttgagaaactcgagggtcctatatgcggatcaattcgcctggcacagcaagctgtttgtttggctgcctgtaagaagctgcacgagatgagtgccttcacagatatgcttttacctgatagaggaagtggggaaggagagaagaccgaaaagaatgaaggtgatccactccctggaacggcacgccatagagagttttatcctgaaggtgttgctgaaattctgagaggagaatggattttatgtggaagagatggctgccaaagcactcaatttattaagttgtatatgtattctgtcaattgtgtggatgtagggacatctaaggacccttcccttgcacaaatatcgaatttttctattatttttggcagtgagctggatgcagaggtatgttccatcatctggaatatcctgaaacttgaatataatgttgtgggttcaccattttttctttctgattttcaggttctgtcgacaccaatggatctctttgttgccaggacgatgataacaaaagcatctcttgtattccgtggaccaattgaagttacagaaagtcagctggttctgctgaagagctttcatgttaggctcatgagcatagtccttgatgttgatgttgatccctcaactacaccttgggatccagcaaaagcatacctctttgtccctgtaggagctgagaagtgtgcaggtgctttaagagagatcgattggactttaatcaacaacattgtgaacactgatgcctggaacaacccacttcagaaggcacgaccagatgtctatctaggcaccaatgagaggacactcggtggagataagagagagtatgggtttggaaaattgcgtaatggaaccgcttttggacagaaggcacatccaacttatggtattagaggagccattgctgactttgatattgttaaagcatctggcttagttccttctcgtgatagagggtattcaagtgactatcagaaccaaggcaagttgttcatggcagattcatgctgggatgctaaagatctttctggaatggttgtcactgctgcccactcaggaaagcggttctatgtggactgtatttgttataacatgaatgcagaaaattcatttcctaggaaggaaggctatctggggcctttggaatatagctcatatgctgattactacaagcaaaaatatggtgtggagctagtgtataagaaacaacctcttatacgtgcacgtggtgtttcctattgcaagaatcttctgtctcctagatttgagcattctgaagctagagaaggtgatttttcggagaacgttgacaaaacatattatgtctatttaccacccgaactttgccttgtgcacccacttcctggatcactcattcgtggagctcagaggttgccatcaataatgagaagggttgagagtatgcttcttgcagttcaattgaaggaaacaattgattatcctgttcccgcagttaagatattagaagccttgactgctgcatcatgccaagaaacattctgctatgaaagagcagagttactgggtgatgcatacctgaaatgggtcgtgagtagattccttttcctaaaatatcctcagaagcatgagggtcagcttacaaggatgaggcagcagatggtcagtaacatggtcctctatcaatttgccctgaataaaactcttcaatcatttattcaagcagatcggtttgccccatcaagatgggcagctcccggagtactacctgtatttgatgaagagacgagagaatctgaaccgtccatctttggtggggagtcaatacctggtaatgaattacagaaggattatgacgatgactacaccgatagcattcaagaggagggcgaaattgatggagattctagctgctatcgtgttctctcaagcaagacattagcagatgttgttgaagcacttattggggtctattatgtagcaggagggaaaattgctgcgaaccacctgatgaagtggattgggatccacgccgaattggatcctcaagagatccctcccccaaagccatataatatacctgagagcatattgaaaggcattgactttgagacattggaaggcatcttgggtaccaaattccagaataaaggttttcttattgaggctataacgcatgcatccaggccatcttctggtgtttcctgttaccagcggttggaatttgttggtgatgctgtgttggatcatctcatcacgaagcatctattttttacatatactgatctacctcctggtcgcttgactgacctgagagctgcggctgttaataatgagaactttgcaagggttgcagttaggcgtaagctgcacggacatcttcgccatggctcatctgcactggaaacacagattcgggagttcgtgaaggatgtccgaaaagagatttcaaaatcaggtttcaattcttttggacttggggattgcaaggctcctaaagttcttggagacattattgagtctatagctggggcagtattcctggatagtgggtatgacacctcggcagtctggaaggtgttccaaccgctcctcgaacctctggtaacgccagagacccttccgatgcatccaattagggagctccaagaacggtgccagcaacaagcagaagggttagaatacaaagcatcccgggcaggcaatgtagctactgtggaagtctttgttgatggtgttcagattggtgtagctcagaatccccagaagaagatggcacagaaattggcggcgaggaacgcactcgtggtcctgaaggagaaggaaactgcagcaaagaaagagacagaaaaggatggtgataagagcaatgctgggttcaccaggcagaccctgaacgacacttgcttgagaaggcagtggccaatgccgcagtacaggtgcataaacgagggcggccctgctcatgctaaaagatttgtgtacgcggttagggtgaatacgtcggatcgtggatggaccgacgagtgcgtcggagagccgatgccgagcgtgaagaaggccaaggactctgccgccatgcttctccttgagcttctgaatcgaagtttccctgataagcctggcggtaagaagtga

SEQ ID NO: 72 - DNA, 5745 bp, Solanum tuberosum
  misc_feature 1..5745: note = ORF of protein Dcl1
  source 1..5745: mol_type = unassigned DNA
atggatggtgatggtggtggagtctctgacaaaccttcttattggattgatgcttatgaggacatctcatgtgatgatctcatccaagattttgttgggtcgaacacagtacccgttgtgccatcgaatcaattgggtccggatgggagtcttgatccttgtttctttggtggtattgatgggattcttgagaaattaaagaatggtgatggttctgtacattctgtggatggaaacaacagtaatgtacatagtgaagctttagctgcacctggaatttgtaaggacaatgggccacaagctaaaaaggatgtggagcacaacaaaggtgatttggggaagaagtctttacaagaaggaaatggattttccaggcataaggaacgaggttgtaatgataaagaagagagaaatggaaaaagggcgcggcttggtgatgatagctaccagaggagaggctgtgatagacctttggctaggaagagacttcgtgagaatgatgagatcaatagagtagggagggatcaaagaaaaagaagagaatatcatggtggtcgtggtggaagagataggaactggagggaagggagtggcttttgggaatgggacaaagaaaggaaagagatggtctatcgtgtgggttcatgggaagctgataggaatcgagaggggaagttaccaactgagagaagccgtgaaccttctggagctattgagaagaaagatgacaaattaaaggaacaagctcccaaggaggaagcacgcaagtatcagttggatgtacttgagcatgccagaaagaacaatacaatcgcttttcttgaaacaggagcaggaaaaacgcttattgccatcctcctcatgaagagtctatgtagtgacttgcaaaagaagaacaagaaaatgctggcagtttttctggttcctaaagttccacttgtttatcagcaagcagaagttattcgtgagcaaactggctatcaagttggccattattgtggtgaaatgggccaagatttttgggatgcacgaaggtggctgcgtgagttcgaaactaaacaggtcctggttatgactgctcagattctcttgaacatattgaggcacagtataataaaaatggaagcaattaatctccttattatggatgaatgccaccatgctgtgaagaagcatccatattctttagtaatgtctgagttctatcatacaacacaaaaggagaaaaggccatctgtttttggcatgactgcttcacctgttaacttgaagggtgtttccagccaagttgattgtgcaataaagattcgtaatttggagactaaattagattctgtcgtctttaccataaaagaccgcaaggagctggagaaacacgtgccaatgccgtcagaagtagtagtggagtatgataaagctgctagtctgtggtcttttcatgaacaaataaaacaaatggaatcagcggttgaagaagctgctctgttaagctccagaagaagtaaatggcaatttatgggggcttgtgatgctggagctagaggagagctgcgccaagtctatggtgtttctgaaagaactgaaagtgatggggctgctaatctaattcaaaagttgagggctatcaactatgctcttggggaactgggacagtggtgtgcctataaggtagcacattcttttttgacagctctacaaaatgatgaaagggcaagctaccagcttgatgtcaagtttcaagaatcctacctggataaggttgtctctctcttgcaatgtcagctatcagagggagctgtagcacaaagtaacttgaatggagagatggataaaggtggcaatccaaatagtgataggcctgacgagatggaggagggcgagcttcttgaaagtcatgttgtatctgttggagagcatgtagatgtgattctaggagctgcggtggctgatgggaaagtgacacctaaagttcaatcattaattaagattcttctcaagtatcagcatactgaggattttcgtgctataatatttgttgaacgcgttgtgactgcattggttcttccaaaggtttttgaggagctcccttccctaagttttatcaattcatcaagtttaatcgggcataacaatagtcaagaaatgcgaacaggtcaaatgcaggacacaatagccaaatttcgagatggtcgtattaatttgttagtcgctactagtgtagctgaggaaggacttgacatccggcagtgcaatgttgtcattcggtttgatctggccaaaactattttggcgtatatccagtctaggggccgtgcccgtaagcctggatcagattatattttgatggttgagagagataattcatcacatgaagcatttctgaggaatgccagaaacagtgaagaaacgttgcggaaagaagcaattgagcggactgatattagtcatctcaaagatgcctccaagttgatctctgcggaggcaccaacagattcagtttatcaggtggagtccactggagctgttgtgagcttaaattctgctgttgggctgatccacttttattgctcccaattaccaagtgacaggtactcgattctccgtcctgagtttataatgaagagtcacgagaagtcaggttgcccaactgaatattcatgcaggcttcaactgccctgcaatgcacctttcgagacacttgatggccctgtatgcagctcaatgcgccttgcccagcaggctgcttgcttggatgcttgcaagaagctccaccagatgggggcatttacggacatgctcttgcctgacaagggaagtggggtggagtcagagaaagttgaacaggatgaagaaggtgttccaattcctggaacttccaggcacagggaattttatcctgaaggtgtagctgatattctgaggggtgattggattttgtcgggaaaggattctcttgtcagctcaaaggtcattcatctttatatgtatgctatcaaatgtgtgaatattggcccctcgaaggacccattcttaactgatgtgtcagagtttgcaatactgtttggcaatgagctggatgcagaggtattatcgatgtcaatggatttatttattgctcgaaccgtagtaacaaaggcgactcttgtcttcagagggccaatagatgttacagagtttcagttggcgtcccttaagagctttcatgtaagaatgatgagcattgttttggatgttgatgttgagccgtccaccactccatgggaccctgcaaaggcatatctatttgcccctgttactggtgatgaatctggagatcctataaaaggtatcaactgggatcatattaaaaaaattactgaaactggtgtatggggcaatcctcttcagagagctcgcccagatgtatatcttggtaccaatgagcgtgctcttggtggagacagaagagaatatggctttgcgaaattgcgacatggcatggccgtcggactgaagtctcatcctacatatggtgttagaggtgctatagcacactatgatctggtccaagcatctggtttggtccctaaccggagtagccttgatgatgttgaagttgatttgaataaagacaagataatgatggctgattgttctcttagagccgaagatattgtaggaagaattgtcactgcagctcactctggaaagagattttatgttgattgcattcgtagtgatatgactgcagagaactcatttccaaggaaagaaggttacctaggtcctcttgagtacagcagttatgctgcttattataagcaaaagtatggagttgatttggtctataaaaagcagcctctaataagagggcgcggtgtttcatattgcaaaaaccttctgtcaccacgatttgaacattcagaagaacacgagggtgaacttgaagaggcaactgacaaaacgtattatgttttcctccctcctgagctctgtgttttgcatccacttcctggatctcttgttcggggtgctcagagattgccctcgatcatgaggaggattgagagcatgctgcttgctgttcagcttaaggaaatgatcggttaccctgtcccagcactgaagatcttggaagcattgaccgccgcttcttgccaggagactttctgctatgaaagagccgaacttcttggagatgcatatctgaaatgggttgttagtcggtatcttttccttaagtatccccagaaacatgaaggccaactcaccaggatgagacagcaaatggtaagcaacatggtgctgtatcaaaatgccttgagtaagggaatccagtcatacatccaagcagatcgattttctccatcgagatgggcagctcctggggtgttgcctgtctatgacgaggacctcaatgaggatgaaacatcgatatttgatcatgaaacagcagaaaatggtactgtagcagcaaaggctctagctggtgatgagtttgaagatgaggaaactgaagaaggtgagcttgataatgattcagggtcatatcgtgtactgtctagcaagactatggctgatgttgtggaagcactgattggagtatattacgttgagggtggaaagtatgccgcaaaccacttcatgaaatggattggggttgaggttgatttcgatttcaatttcaaagagacagaatactctattaggtcttgtagcattcctgagaatgtactcaagagtgttgactttgatgcattacaaggtgctttgaacatcagcttcaatgataaaggcctactgctagaagcaattacccatgcttcacgcccatcttctggggtctcctgttatcaaagactggagtttgtgggtgatgcagtattagatcatctcatcacgaggcacttgttctttacatacacagatcttcccccaggccgattgaccgacttgcgagctgcggctgtcaacaatgaaaattttgcacgtgttgctgttaagcacagtcttcatttacaccttcgtcatgcatccagtgctctagaaaaacagattcgtgactttgtgaatgaggttaaaaatgaactatcaaaaccaggtttcaactcctttggtttgggagattgcaaagctcctaaagttcttggtgatattgttgaatcaattgctggggctatttttctagacagtgggtgtaatactaaatctgtatgggaggtttttcaaccactgttgcatccaatggttacaccagaaacactgcccatgcatccagttcgagaattgcaagaacgttgtcaacaagaagctcaaggcttagagtacaaagcaactcgaagtgggaatatggctacagttgaagtatatgtggatggtcttcaggttggaatggcgcagaatcctcagaagaaaatggcacagaagttggcggccaggaatgcccttgttgtgttgaaagagagggaggaagctgaagctaagaaggctgatgatgcaaagaaaaagaagaatggcaacccgagttttactaggcagacactaaatgatatatgcttacgcaaaaattggcctatgccactgtatcgatgtgtgcacgagggtggtccagcacatgcaaagaggtttacatatggagtacgcgtaaatatttcagacaaaggatttacagatgaatgtatcggggaacctatgccgagtgtaaagaaagccaaggattccgcagcagctcttctgttggaactcttaaatagacggtactcataa

SEQ ID NO: 73 - DNA, 5643 bp, Medicago truncatula
  misc_feature 1..5643: note = ORF of protein Dcl1
  source 1..5643: mol_type = unassigned DNA
atggaggatgatgccgatgccgctgctgccgccgctgccgtgaataacaacaatagggtttcttcgtcaaatccttactggctggatgcctgtgaagatatttctatatcttgtgatgatatcattgattttgatgtttctaatgattctgaccaacaacccaacaacaacaaccaagacttttttggtggtattgatcgaatctttgacagcatcaaaaacggcgctggtctccctgatcatcctcctgcttctgctgctgatgatgggaatcaactacctcctactcatcttcaactagaagaacaacaccaacaggtagaagaagaagaacaagaacaagaaaggttcaacaaaagggctcgcctcaccgaaggagaaaggagaatcaatcatcaacccaacaacaacaacaaggacaggtatgagaggtgtttcaacaataataggaagagaccaaggaataataataatcataatcattcctttaaaagagatgtcaagggttattgggagagggataagtcttctaatgacttggtctttcgctccggaacttgggaacccaatcttcatacttcccaacacaaaatggacatagacaaccccatcaaacaggaaaaacccaaggagactgttcctgttcctgaggaaaaagccagacagtatcagttggatgttcttgagcaggcaaagaccagaaatacaatagcttttctagaaactggagctggcaaaaccctcattgccgttcttctcatcaagtctatacatgagacattacatctccaaaataagaaaatgcttgctgtgtttttggttccaaaagttccacttgtttaccagcaagctgaggtaatccgtgagcgcactggatatcaagtggggcactattgtggagaaatggggcaggatttctgggatgctcgaaggtggcagcgtgaatttgacaccaagcatgttttagtcatgaccgctcaaatacttttgaacattttgaggcatagcatcataaaaatggaagcaatcaatctcctgattcttgatgagtgccatcatgctgtgaagaaacacccatattcgctggtgatgtctgagttctaccatacaacgcccaaagaaaagaggccatctgtatttggaatgactgcttctcctgttaacttaaagggtgtttccagccaagtagactgtgcaataaaaattcgtaatcttgaaagtaagctagattcaatagtttgtaccattaaagatcgtaaagagctagagaaacacgtgcctatgccctctgagattgtggtgaagtatgataaagcggccagtctatgttatttacatgagcaaataaagcagatggagactgaagttgaggaagctgcaaaatctagttcaagaagaagcaaatggcagtttatgggagctagagatgctggagctaaggaagagctgcgccaagtatatggtgtttctgaaagaacagaaagtgatggagctgcaaacctaattcagaagttgagagctgttaattatgcacttggtgaattgggacagtggtgtgcatataaggttgcacaatcttttcttgcagctctgcaaaatgatgaaagggcaaactatcaacttgatgtcaagtttcaggaatcttatctgagtaaagttgtgtctcttctgaaatgtcagctgtcagagggtgccatttctgacaaaaatgatggagtagatgactcacaaaatggtgcagctcacagtgggtctgatgaacatgaagagattgaagaaggagagcttccagacagtcacgttgtctctggtggggagcatgttgatgtcataataggtgctgctgtggctgatggcaaggtgacccccaaggtgcaggctttaatcaaaatactcctcaagtatcagaacacagacgacttccgtgcaatcatctttgttgaacgtgttgtgtctgcattagttctacccaaggtttttgccgagcttccatctcttagttttgtcaagtgtgcaagcttgattggccacaacaatagtcaggaaatgcggacccaccaaatgcatgatacaattgccaagttccgtgatgggcgggtcacgttgttagttgccactagtgttgctgaagaaggtcttgatatccggcaatgtaacgttgttattcgctttgatcttgcgaagactgttctggcatacattcagtctaggggacgtgctagaaagcctgggtctgattacatcctaatggttgagaggggaaatttgtcacatgaagctttcctaaagaatgccaggaacagtgaggaaactttgcggagagaagcaatagagagaacagaccttagtcatctgaaagacagttcaaggttgatttctgtagatacccatccagaaacaatgtaccaggtgaaatctaccggtgcagtagttagcttaaattccgctgtaggtcttgtccacttctattgctcccagctacctagtgacagatattcaattcttcgtcccgagtttattatggagaagcatgaaaactccggaggtcccactgaatattcttgcaggcttcaactgccatgtaatgcaccatttgaaaatctggagggtcctatctgcagttcaatccgcttggcacaacaggctgtttgtctagctgcttgcagaaaattgcacgaaatgggagcattcactgacatgctattgccggataaaggaagtggggaagaaaaagaaaaggctgagcaaaatgatgaaggggatgcacttccagggactgctagacatagggagttctatcctgaaggtgtggctgacatactgaagggagaatggatcttatctggaaaagatgcttgtaacaactccaaattgcttcatctttatatgtataccataaagtgcgagaatgtaggccattctaaggatccgttcttgactcaggtttcagattttgcagtactttttggcactgaactggatgcagaggtcttatcgatgtcgatggatctatttatcgctcgaaccgtgactacgaaggcgtctcttgtctttagagagtcaatagatatcactgagagtcagttgacgtccctgaaaagctttcatgtaaggttaatgagcattgtcttggatgtggatgttgaaccatctaccactccttgggatcctgcaaaagcatatctgtttgtcccaatggttggcgataagtcttcagatcctatgaaccaaattgactggcatctggttgagacaataattggagccgatgcatggaaaaatcccctccagaaggctcgaccagatgtttaccttggcactaatgagagaactttaggtggtgacagaagggaatacggatttgggaaattgcgtcatggcttggcttttgggcaaaaatcacatccaacctatggaatcagaggagctgtggcccagtttgatgtagtgaaagcttcaggattggttcccaacagagattccatggaaacacaaaatcatatcaatatgactaccaaaggaaaattgatgatggcagatacatgtaccagtccagatgatctagtagggagaattgtaactgctgctcattcagggaagaggttttatgttgattctatacgctatgaaatgacagcagagaactctttccctaggaaagaaggttatcttggtcctctggaatacagttcatatgctgattactacaagcaaaaatatggtgttgatttggcttacaagcaacaacctcttataagagggcgtggtgtatcatactgcaagaatcttttgtcccctcgatttgaacacagtgaaggacatgaagatgaaactgaagagacccacgacaaaacttactatgttttccttcctccagagctatgcctagtacacccactgcctggatcactcgttcgtggtgctcagaggctgccttcaataatgaggagggttgaaagcatgctgcttgcagttcagcttaagaatatgataaattatcctgtccaggcttcaaagatcttggaagccttgaccgctgcctcttgccaggagactttctgctatgaaagggcagagcttctaggagatgcttacctgaaatgggttgttagtcgttttcttttcctaaaacatccacagaaacatgaaggccagcttactaggatgagacaacaaatggtcagtaacatggtcttatatcggtacgcattaagtaaaggtcttcaatcatatatcctagcagatcgttttgccccatccagatgggcagctcctggggtgctacctgtctttgatgaggatacaaaggatgaggagtcgtctttatttgaccaggagcgttccatttttaaggccgagaggatggacaacactgatgaatttgaggatgaaatggaagatggtgagcttgagagtgattcaagttcttaccgagttctgtccagcaagacacttgcagatgttgtggaagcactgattggggtatattatgttgaaggtggtaagaatgctgctaaccaccttatgaaatggattggtattcacatagagatcgatcctgatgagatggagtgtataacgaagccatccaatgttccagacagcatacttaggagtgttgattttgatgctttagaaggtgctctaaatattaagtttaaagacaaggggctgctgatagaatccattactcatgcctccagaccatcttcaggagtatcctgttatcagcggttggagtttgttggtgatgctgtcttggaccatcttataaccaggcacttatttttctcgtacacagatctgccaccaggacgtctgactgaccttcgagctgctgctgtaaacaatgaaaattttgcacgtgttacagttaaacacaatcttcacctgcatctgcggcatgggtccagtgccctagaaaaacagattaaagattttgtgaaggaagttcaagatgaattgtcaaaaccagggtttaactcttttggtcttggagactgcaaagctcccaaggtccttggtgacatccttgaatctattgctggtgccatttttcttgacagtgggaggaatactgctgttgtttggaaggtttttcagcctcttttgcatcctatggttacaccagagactttgccaatgcatcctgtgcgtgagcttcaagagcgatgccagcagcaggctgaaggcttggagtatagagcaagtcgtgctggaaatttagccactgttgaagtattcattgatggggtacaagtaggggctgctcaaaatccacagaagaaaatggcacagaaattagctgcccgaaatgcccttgctgctttgaaagagaaggaagagagtaaaattcaggagaaaaatgatgaaaaggagacaaagaatgggaaccagacatttactagacaaacattaaatgatatatgtttgcgaagaaactggcctatgccattctaccggtgtgtgagtgagggtggtcctgcacatgcaaagaggtttacatttgcagtgcgagtgaatactactgataaaggttggacggatgaatgtgttggagaggcaatgccaagcgtcaagaaggccaaagactcagctgctgttcttctcttggaactaataaacaaattatactcgtcatag

SEQ ID NO: 74 - DNA, 969 bp, Triticum aestivum
  misc_feature 1..969: note = ORF of protein Dcl1
  source 1..969: mol_type = unassigned DNA
atgagaagggttgagagtatgcttcttgcagttcaattgaaggaaacaattgattatcctgttcctgcagttaagatattagaagccttgactgctgcatcatgccaggaaacattctgctatgaaagagcagagttactgggtgatgcatacctgaaatgggttgtgagtagattccttttcctaaaatatcctcagaagcatgagggacagcttacaaggatgcgacagcagatggtcagtaacatggtcctctaccaatttgccctgaataaaactcttcaatcgtatattcaagcagatcggtttgccccatcaagatgggcagctccaggagtactacctgtatttgatgaggagacgagagaatctgaaccgtccatctttggtgaggggtcaatacctggtaatgaagtacagaaggattatgatgatgactacacacatagcattcaagaggagggtgaaattgacggagattctagctgctatcgtgttctctcaagcaagactctagcagatgtcgttgaagcacttattggggtctattatgtagcaggagggaaaattgctgcaaaccacctgatgaggtggattgggattcatgctgagttggatcctcaagagatccctcctccgaagccatataatatacctgagagcatattgaaaggcattgactttgagacattggaaggcgtcttgggtatgaaattccagaataaaggttttcttattgaggctataacgcatgcatccaggccatcttctggtgtttcctgttaccagcggctggaatttgttggtgatgctgtgttggatcatctcatcacaaagcatctattttttacatatactgatctacctccaggtcgcttgactgacctgagagctgcagctgttaataatgaaaactttgcaagggttgcagttagacgtaagctgcacggacatcttcgccatggatca

SEQ ID NO: 75 - DNA, 5340 bp, Triticum aestivum
  misc_feature 1..5340: note = ORF of protein Dcl1
  misc_feature 213..280: note = n is a, c, g, or t
  source 1..5340: mol_type = unassigned DNA
acggcggtcgcggcggggagtggagagacgggaagcgtcctcgcctcgcccctggggctcacggggagccgcgccctgattcgcggcggcggccgatgcttccgccgccctcgcgtggctgggaggaacggcgcgggaggcgcgacttcgttgacaggcctcgcaagcgcgaccgcgacagtaaccctgcccaccgccgcgaggctcgagggnnnnnnnnnnnnnnnnnnnnnnnnnnnnnnnnnnnnnnnnnnnnnnnnnnnnnnnnnnnnnnnnnnnnccaggacacacaacggaggctccgtggagaagactgcagaggcggataaggcggcttccgcccagaaggagaagcctctcgccgaggagcaggcgagacactaccagttggaggtgcttgagcaagcaaagagcaggaacaccatagccttcctcgagaccggcgcggggaagaccctcattgctgtgctgctcatcaaaagcatatgtgacaaaatgatgaaggagaacaggaaaatacttgctgtgttcttggttcccaaggtgcctcttgtgtatcagcaagctgaggtgatacgcgagaggactggctatcgggtcgggcactactgtggagagatgggtcaggatttctgggatgctagaaagtggcagcgcgagtttgattcgaaacaggtactagttatgacagctcaaattttgttaaacatcctcaggcacagtatcattaaaatggatgccatacatcttttaattttagacgagtgccatcatgcagtgaaaaaacatccatattctctagtgatgtcagaattttatcacacaactccgaaggataagagaccagttgtttttggtatgacagcttctcctgtcaacctaaagggagtcactagccaagaagattgtgccatcaagataagaaatcttgagagtaaactggattccattgtttgtactatcaaagatcggaaagagcttgagaaacatgtccccatgccttttgaagtcgttgtccaatatgacaaagcagccaccctttgttccctgcatgagcagatcaaacaaatggaagctgctgttgaagaagctgcactctgtagctctaagagaacaaagtggcagtttatgggagccagggatgctgggtctagagatgagctgcgccttgtctatggtgtttctgagcgtacagaaagtgatggtgcagctaatttaatccagaagttgagagctataaattatgctttaggtgaactaggacaatggtgtgcttataaggttgcacagtcctttttgacagtattgcagaatgatgaaagggcaaactatcaggttgatgtaaagtttcaagagtcgcatttaaagaaggttgttgatttattgcactgccagttgactgagggagctgcagtgaaaagcgagacgaatgatgttgagatgcataacactgaaaatcacaacccaagtgaacttgaagacggggagctgcctgatagccatgctgtttctgtaggtgaacatgtagatgaggtcatcggagctgccgtggcagatgggaaagttacccctagggtgcaggctttaatcaaaatccttctcaagtatcagcatactgacgatttccgtgctatcatctttgtggagcgagttgtcacggcattagttcttcctaaggtgcttgcagagcttccatctttaagctttatccgatgtgcaagcttgataggtcataacaacaatcaagagatgcgcacaagccaaatgcaggataccattgcaaaatttcgtgatggccgagttacactgttagtcgcgactagtgttgctgaggaaggacttgatattcgacagtgcaatgttgtcattcgctttgaccttgcaaagacggtcttggcctacgtacagtctcgtggtcgtgcaagaaagcctgggtctgactacatattgatggttgaaagggaaaatctggcacatgaaactttcttgaggaatgctcgaaatagtgaggagacgttgagaaaagaggctatagaaagaactgatctcagtcatcttgatggtactctgctaaattccattgatacatcacctgattcgatgtatcaggtcgaatcaacaggtgctgttgtcagcttgaattctgcagttgggctcgtacatttttactgttcacagctaccaagtgacaggtactcaattcttcgtccagaatttattatgcaaaaacatgagaagccagggggttccatggagtattcctgcaaacttcaacttccctgtaatgctccatttgagaaacttgagggtcctatatgcggctcaattcgtctggcacaacaagctgtttgtttggctgcctgtaagaagctgcatgagatgaatgccttcacagatatgcttttacctgatagaggaagtggagaaggagagaagaccgaacagaatgaaggtgatccactccctggaacggcacgccatagagagttttatcctgaaggtgttgctgaaattttgagaggagaatggattttatgtggaagagatggctgccaaagcactcaatttattaagttgtatatgtattctgtcaattgtgtggacgtaggggcatctaaggaccctttccttgcacaactttcgaatttttctattatttttggcagtgagttggatgcagaggttctatcgacgccaatggatctctttgttgccaggacaatgataacaaaggcatctcttgtattccgtggaccaattgaagttacagaaagtcagctggttctgctgaagagctttcatgttaggctcatgagcatagtccttgatgttgatgttgatccctcaactacaccttgggatccagcaaaagcatacctgtttgtccctgtaggaactgagaagtgcacagatgctttaagagagatcgattggactttggtcaacagcattgtgaatactgatgcctggaataatccacttcaaaaggcacgaccagatgtctacctaggcaccaatgagaggacacttggtggagataagagagagtatgggtttggaaaattgcgtaatggaaccgcttttggacagaaggcacatccgacttatggtattagaggagccattgctgactttgatattgtcaaagcatctggattagttccttctcgtgatagaggttattcaagtgactatcagaatcaaggcaagttgttcatggcagattcatgctgggatgctaaagatctttctggaatggttgtcactgctgcccactcaggaaagcggttctatgtggactgtatttgctataacatgaatgcagaaaattcatttcctaggaaggaaggctacttggggcctctggaatatagctcatatgccgattactacaagcagaaatatggtgtggagctagtgtataagaaacaacctcttatacgtgcacgtggtgtttcctattgcaagaatcttctgtctcctagatttgagcattctgaagctagagaaggtgacttttcggagaacgttgacaaaacatactatgtgtatttaccacctgaactttgccttgtgcacccccttcctggatcacttattcgtggagctcaaaggttgccatcgataatgagaagggttgagagtatgcttcttgcggttcaattgaaggaaacaattgattatcctgttcctgcagttaagatattagaagccttgactgctgcatcatgccaggaaacattctgctatgaaagagcagagttactgggtgatgcatacctgaaatgggttgtgagtagattccttttcctaaaatatcctcagaagcatgagggacagcttacaaggatgcgacagcagatggtcagtaacatggtcctctaccaatttgccctgaataaaactcttcaatcgtatattcaagcagatcggtttgccccatcaagatgggcagctccaggagtactacctgtatttgatgaggagactagagaatctgaaccgtccatctttggtgaggggtcaatacctggtaatgaagtacagaaggattatgacgatgactacacggatagcattcaagaggagggtgaaattgacggagattctagctgctatcgtgttctctcaagcaagactctagcagatgtcgttgaagcacttattggggtctattatgtagcaggagggaaaattgctgcaaaccacctaatgaagtggattgggattcatgccgagttggatcctcaagagatccctcctccgaagccatataatatacctgagagcatattgaaaggcattgactttgagacattggaaggcatcttgggtatgaaattccagaataaaggttttattattgaggctataacgcatgcatccaggccatcttctggtgtttcctgttaccagcggttggaatttgttggtgatgctgtgttggatcatctcatcacaaagcatctattttttacatatactgatctacctcctggtcgcttgactgacctgagagctgcagctgttaataatgaaaactttgcaagggttgcagttagacgtaagctgcacggacatcttcgtcatggatcatctgcactggaaaaacagattcgggagttcgtgaaggatgtccgagaagagatttcaaaatcaggtttcaattcttttggactaggggactgcaaggctcctaaagttcttggagacattattgagtctattgctggggcagtattcctggatagtgggtatgacacctcggcagtctggaaggtgttccaaccgctccttgaacctctggtaacgccagagacccttccgatgcatccgattagggagctccaagaacggtgccagcaacaagcagaaggcttagaatacaaagcatcccgggcaggcaatgtagctactgttgaagtctttgttgatggtgttcagattggtgtagctcagaatccccagaagaaaatggcacagaagttggcggcgaggaacgcacttgtggtcctgaaggagaaggaaaccgcagcaaagaaagagaccgaaaaggacggtgataagaacaatgccgggttcaccaggcagaccctgaacgacacttgcttgagaaggcagtggccgatgccacagtacaggtgcataaacgagggcggccctgctcatgctaaaagatttgtctacgcggtcagggtcaatacatccgatcgcggatggaccgacgagtgcgtcggagagccgatgccgagcgtgaagaaggccaaggactctgccgcgatgcttctcctcgagcttctgaaccgaagtttccctgaaaagcctgacggtaagaagtga

SEQ ID NO: 76 - DNA, 5025 bp, Triticum aestivum
  misc_feature 1..5025: note = ORF of protein Dcl1
  source 1..5025: mol_type = unassigned DNA
atggtgttccgccctggcacctgggagcaggagtctgagcgcgagggaaagcgtgccaggacacagaacggaggctccgtggagaagaatgcagaggcggataaggcggcttctgcccagaaggagaagcctctcgcagaggagcaggcgagacattaccagttggaggtgcttgagcaagcaaagagcagaaacaccatagccttcctcgagaccggcgcggggaagactctcattgctgtgctgctcatcaaaagcatatgtgacaaaatgatgaaggagaacaggaaaatacttgctgtgttcttggttcccaaggtgcctcttgtgtatcaggtactagttatgacagctcaaattttgttaaacatcctcaggcacagtatcattaaaatggatgccatacatcttttaattttagatgagtgccatcatgcagtgaaaaaacatccatattctttagtgatgtcagaattttatcacacaactccgaaggataagaggccagttgtttttggcatgacagcttctcctgtcaacctaaagggagtcactagccaagaagattgtgccatcaagataagaaatcttgagagtaaactggattccattgtttgtactattaaagatcggaaagagcttgagaaacatgtccccatgccttttgaagtcgttgtccagtatgacaaagcagccaccctttgttccctgcatgagcagatcaaacaaatggaagttgctgttgaagaagctgcactctgtagctctaagagaacaaagtggcagtttatgggagccagggatgctgggtctagagatgagctgcgccttgtctatggtgtttctgagcgtacagaaagtgatggcgcagctaatttaatccagaagttgagagctataaattatgctttaggtgaactaggtcaatggtgtgcttataaggttgcagtgtcctttttgacagtattgcagaatgatgaaagggcaaactatcaggttgatgtaaagtttcaagagtcacatttaaagaaggttgttgctttattgcactgccagttgactgagggagctgcagtgaaaagtgagacgaatgatgttgagatgcataacagtgaaaatcacaacccaagtgaacttgaagatggggagctgcctgatagccatgctgtttctgtaggtgaacatgtagatgaggtcatcggagctgccgtggcagatgggaaagttacccctagggtgcaggctttaatcaaaatccttctcaagtatcagcataccgaggatttccgtgccatcatctttgtggagcgagttgtcacggcattagttcttcctaaggtgcttgcagagcttccatctttaagctttatccgatgtgcaagcttgataggtcataacaacaatcaagagatgcgcacaagccaaatgcaggataccattgcaaaatttcgtgatggccgtgttacactgttagttgcgactagtgttgctgaggaaggacttgatattcgacagtgcaatgttgtcattcgctttgaccttgcaaagacagtcttggcctatgtacagtctcgtggtcgtgccagaaagcctggatctgactacatattgatggttgaaaggggaaatttggcacatgaaactttcttgaggaatgctcgaaatagtgaggagacgttgagaaaagaggctatagaaagaactgatctcagtcatcttgatggtactctgttaaattccattgatacatcacctgattcgatgtaccaggtcgaatccacaggtgctgttgtcagcttgaattctgcagttgggctcgtacatttttactgttcacagctaccaagtgacaggtactcaattcttcatccagaatttattatgcagaaacatgagaagccagggggttccatggaatattcttgcaaacttcaacttccctgtaatgctccatttgagaaacttgagggtcctatatgcggctcaattcgtctggcacagcaagctgtttgtttggctgcctgtaagaagctgcatgagatgaatgccttcacagatatgcttttacctgatagaggaagtggggaaggagagaagaccgaacagaatgaaggtgatccactccctggaacggcacgccatagagagttttatcctgaaggtgttgctgaaattttgagaggagaatggattttatgtggaagagatggctgccaaagcactcaatttgttaagttgtatatgtattctgtgaattgtgtggatgtagggacatctaaggacccttcccttgcacaactttcgaatttttctattatttttggcagtgagctggatgcagaggttttatcgacaccaatggatctctttgttgccaggacaatgataacaaaggcatctcttgtattccgtggaccaattgaagttacagaaagtcaggttgatctgcttgagaagttccaactggttctgctgaagagctttcatgttaggctcatgagcatagtccttgatgttgatgttgatccctcaactacaccttgggatccagcaaaagcatacctctttgtccctgtaggagctgagaagtgtacagatgctttaagagagatcgattggactttagtcaacagcattgtgaatactgatgcctggaataatccacttcagaaggcacgaccagatgtctacctaggcaccaatgagaggacactcggtggagaaaagagagagtatgggtttggaaaattgcgtaatggaacagcttttggacagaaggcacatccgacttatggtatcagaggagccattgctgactttgatatcgtcaaagcatctggattagttccttctcgtgatagagggtattcaagcgagtatcagaaccaaggcaagttgttcatggcagattcatgctgggatgctaaagacctttctgggatggttgtcactgctgcccactcaggaaagcggttctatgtggactgtatttgctataacatgaatgcagaaaattcatttcctaggaaggaaggctacctggggcctttggaatatagctcatatgccgattactacaagcagaaatatggtgtggagctagtgtataagaaacagcctcttatacgtgcacgtggtgtttcctattgcaagaatcttctgtctcctagatttgagcattctgaagctagagaaggtgacttttcggagaacgttgacaaaacatactatgtctatttacctcccgaactctgtcttgtgcacccacttcctggatcacttattcgtggagctcagaggttgccatcaataatgagaagggttgagagtatgcttcttgcagttcaattgaaggaaacaattgattatcctgttcctgcagttaagatattagaagccttgactgctgcatcatgccaggaaacattctgctatgaaagagcagagttactgggtgatgcatacctgaaatgggttgtgagtagattccttttcctaaaatatcctcagaagcatgagggacagcttacaaggatgcggcagcagatggtcagtaacatggtcctctaccaatttgccctgaataaaactcttcagtcgtatattcaagcagatcggtttgccccatcaagatgggcagctccaggagtactgcctgtatttgatgaggagacaagagaatctgaaccgtccatctttggtgaggagtcaatacctggtaatgaattacagaaggattatgatgatgactacacagataacattcaagaggagggtgaaattgatggagattccagctgctatcgtgttctctcaagcaagactctagcagatgtcgttgaagcacttattggggtctattatgtagcaggagggaaaattgctgcaaaccacttgatgaagtggattgggattcatgccgagttggatcctcaagagatccctcccccgaagccatataatatacctgagagcatattgaaaggcatcgagtttgagacattggaaggcatcttgggtatgaaattccagaataaaggttttcttattgaggctataacgcatgcatccaggccatcttctggcgtttcctgttaccagcggttggaatttgttggtgatgctgtgttggatcatctcatcacaaagcatttattttttacatatactgatctacctcctggtcgcttgactgacctgagagctgcagctgttaataatgaaaactttgcaagggttgcagttaggcgtaagctgcacacacatcttcgccatggatcatctgcactggaaaaacagattcgggagttcgtgaaggatgtccgagaagagatttcaaaatcaggtttcaattcttttggactaggggactgcaaggctcctaaagttcttggagacattattgagtctattgctggtgcagtattcctggatagtgggtatgacacctcggcagtctggaaggtattccaaccgctcctcgaacctctggtaacgccagagacccttccaatgcatccaattagagagctccaagaacggtgccagcaacaagcagaaggcttagaatacaaagcatcccgggcaggcaatgtagctaccgttgaagtctttgttgatggtgttcagattggtgtagctcaaaacccacagaagaagatggcacagaagttggcggcgagaaacgcactagtggtcctaaaggagaaggaaaccgcagcaaagaaagagaccgaaaaggccggtgataagagcaatgctgggttcaccaggcagaacctgaacgacacttgcttgaaaaggcagtggccaatgccacagtacaggtgcataaatgagggcggccctgctcatgctaaaagatttgtctacgcggttagggttaatacatccgaccgcggatggaccgacgagtgcatcggggagccgatgccgagcgtgaagaaggccaaggactctgccgccatgcttctcctcgagcttctgaatagaagtttccctgataagcccgacggtaagaagtga

SEQ ID NO: 77 - DNA, 1122 bp, Triticum aestivum
  misc_feature 1..1122: note = ORF of protein Dcl1
  source 1..1122: mol_type = unassigned DNA
atgtattctgtgaattgtgtggacgtagggacatctaaggacccttcccttgcacaactttcgaatttttctattatttttggcagtgagctggatgcagaggttctatcgacgccaatggatctctttgttgccaggacaatgataacaaaggcatctcttgtattccgtggaccaattgaagttacagaaagtcagctggttctgctgaagggctttcatgttaggctcatgagcatagtccttgatgttgatgttgatccctcaactacaccgtgggatccagcaaaagcatacctgtttgtccctgtaggagctgagaagtgtacagatgctttaagagagatcgattggactttagtcaacaacattgtgaatactgatgcctggaataatccacttcagaaggcacgaccagatgtctacctaggcaccaatgagaggacacttggtggagataagagagagtatgggtttggaaaattgcgtaatggaactgcttttggacagaaggcacatccgacttatggtattagaggagccattgctgactttgatattgtcaaagcatctggattagttccttctcgtgatagaggttattcaagtgactatcagaatcaaggcaagttgttcatggcagattcatgctgggatgctaaagatctttctggaatggttgtcactgctgcccactcaggaaagcggttctatgtggactgtatttgctataacatgaatgcagagaattcatttcctaggaaggaaggctacctggggcctttggaatatagctcatatgccgattactacaagcagaaatatggtgtggagctagtgtataagaaacaacctcttatacgtgcacgtggtgtttcctattgcaagaatcttctgtctcctagatttgagcattctgaagctagagaaggtgacttttcggagaacgttgacaaaacatactatgtgtatttaccacctgaactttgccttgtgcacccacttcctggatcacttattcgtggagctcaaaggttgccatcgataatgagaagggttgagagtatgcttcttgcagttcaattgaaggaaacaattgattatcctgttcctgcagttaag

SEQ ID NO: 78 - DNA, 5757 bp, Lolium perenne
  misc_feature 1..5757: note = ORF of protein Dcl1
  source 1..5757: mol_type = unassigned DNA
atggcgggcggcggcggcgtcacgtactggtacgacgcgtgcgaggacgacgacgacgacgaggacagctcctcgctgctctgcggcatcgacttcgccgcgtccgctgacttcgacccggggttcttgcccaacttgggcctcgacagcggcggcgtcgacgacggcttcgtcgccgaaatcgaccggattctcgagagcatcaacgccgacaccgcccctgcgcccgcgcccgcgcccgcgccgccaccgcccccgcccccgtctcagcccgtggctacgccaccgccgcagccacagccgcagcaaaaggaggctcctttggccgtggcccacaatgccctagcggtggtggcccctgcctcggggcagggggtcgagccgaggaaggagacgcggagggagtcccacggcagctccgccaatggcggcggggattacaggggcgggaagcgccctcgcctcgtctccgggcctgacagggcgccgcgccccgacccgcggcggcgccccccgcccccgccctcgcgcgggtgggatgaccggcgcgggaggcgcgacttcgacaggcctccgcacaagcgcgaccgcgaccaccaccaccgccgcgaggcccgggggttctgggagcgggaccgcggcgggaagatggtgttccgccccggcgcctgggaccaggaggagtccggccgcgacgcgaagcgcgccagggcagagaacggaggcaccgtggggaagaaagcagaggcggctgctgcacagaaggagaagcccctcaccgaggagcatgcgaggcagtaccagctggaggtgctcgagcaggcaaagagcaggaacaccattgccttcctcgagaccggcgccgggaagacgctcattgccgtgctgctcgtcaagagtttatgcgacaagatgctcaaggagaacaagaagatacttgctgtgttcttggttcccaaggtgcctcttgtgtaccagcaagctgaggtgatccgtgacaggactggctatcgggttgggcattactgcggagagatgggtcaggatttttgggattctaggaagtggcagcgcgagtttgattctaaacaggtacttgttatgacagctcaaattttgttaaacatcctccggcacagtattattaaaatggatgcgatacatcttctaattttagacgagtgccatcatgcagtgaaaaaacatccatattctctagtgatgtcagaattttatcatacaactcccaaggataagagaccagttgtttttggtatgacagcttctcctgtcaacctgaagggagtcaccagccaagaggactgtgccatcaagataaggaatcttgaaactaaattagattccatagtttgtactatcaaagatcggaaggagcttgagaagcatgttcccatgcctttggaagtcgtcgtgcaatatgacaaagcagccactctttgttccttgcacgagaagatcaaacaaatggaaactgcagttgaggaagctgcactctctagctctaagagaagcaagtggcagttcatgggagccagggatgctgggtctagagatgagctgcgccttgtctatggtgtttctgagcgtacagaaagtgatggtgcagctaatttattccagaaattgagagctataaactatgctttaggtgaactaggacaatggtgtgcttacaaggttgcacagtccttcttgacagcgttacagaatgatgaaagggcaaactatcaggttgatgtaaagtttcaagagtcgtatttaaaaaaggttgttgatttattacactgccagttgactgaaggagctgctatgagaagcgagacaaatgatgttgagatgcataatgctgaaaacgacaatccaagtgagctagaggacggggagctgcctgatagccatgctgtttctgtaggtgaacatgtagatgaagtcatcggagccgccgtggcagatgggaaggttacccctcgggtgcaaagtttgatccaaatacttctcaagtatcagcataccaaggatttccgtgctatcatctttgtggagcgagttgtcaccgcattagttcttcctaaggtgttggcagagcttccatctctaagctttatacgatgtgcaagcttgataggccataacaacaatcaagagatgcgcacaagccaaatgcaggatacaattgcaaagtttcgggatggccgtgttacgctgcttgttgcgactagtgttgcagaggaaggtcttgatattcgacagtgcaatgttgtcatccgctttgaccttgcgaagactgtcttggcctacatacagtctcggggtcgtgcaagaaagcctggatctgattacatattgatggttgaaagggaaaatatgtcacatgaaactttcttgaggaattgtcgtaatagtgaggagacattgagaaaagaagctatagaaagaactgatctcactcatcttgatggcactcctgcgttaaatccaattgatacctcccctgattcgatgtaccaggtcgaatcaacaggtgctgttgttagcttgaattctgcagttggcctcgtacatttttactgctcacagcttccgagtgacaggtactcaattcttcgtccggaattttttatgcaaaggcacgagaagccagggggttcagtggaatattcttgcaaaattcaactgccctgtaatgctccatttgagaaacttgagggtccaatatgcagttccattcgtctggcacagcaagctgtttgtttggctgcgtgcaagaagctacatgagatgagtgccttcacagatatgcttttacctgatagaggaagtgaggaacgagaaaagactgaacagaatgatgaaggtgatccactccctggtacagcaaggcatagagagttttatcctgaaggagttgccgaaattctaagaggagaatggattttgtctggaaaaaatagctgccagagctcgcagtttattaagctgtacatgtattctgtgagctgcatagatgttgggacatctaaggaccctttccttgcacagctttcgaatttttctattattttcggcaatgagctggatgcagaggttttatcgacgacaattgatctctttgttgccaggacaatgataacaaaggcatctcttgtattccgtggaccaattgagattacagaaagtcagctggtcttgctgaagagctttcatgttaggctcatgagcatagtccttgatgtggatgttgatcccttaacgacaccttgggatccggcaaaagcatacctctttgtgcctgtaggagctgagaagtgtagtacagacgctttaagagagatcgattggaccttaattaacagcattgtgaacactgaagcttggaataacccacttcagaaggcacggccagatgtctacctaggaaccaaagagcggacacttggtggagataagaaagagtatgggttcggcaaattgcgtaatggcaccacttttggacagaaggctcatccgacttatggcataagaggagccattgcacagtttgatattgtcaaagcatctggattagttcctggtcgtgatagagggtattttaatgactatcagaatcaagggaagttgttcatggcagattcatgctgggatgctaaggatcttgctggaatggttgtgactgctgcccactcaggaaagaggttctatgtggactgtatctgttataacatgaatgcagaaaattcgtttcctaggaaggagggctacctggggcctttggagtatggttcgtatgctgactactacaagcagaagtatggcgtagagctagtgtataagaagcaacctcttatacgtgcacgaggtgtttcctattgcaagaatcttttgtctcccagatttgagcattcggaagctaaagaaggtgaccttccagagaaccttgacaagacgtactatgtgtatttaccacccgaactgtgctttgtacacccacttcctggatcacttgttcgtggagcccagaggttgccatcaataatgagaagggtggagagcatgcttcttgcagttcaattgaaggaaataattgactaccctgttcctgcaactaagatattagaagccttgactgctgcatcatgccaggaaacattctgctatgaaagagcagagttactgggtgatgcataccttaaatgggttgtgagtagattccttttcctaaaatatccccagaagcacgagggacagcttacaaggatgcggcagcagatggttagtaacatggtcctgtacgaatttgccctgaataagactcttcaatcgtatattcaagcagatcggtttgccccatcaagatgggcagctccgggagtgctacctgtatttgatgaggaaacaagagaatccgagccgtccatctttggcgaagaatcaattcctggtaatgaattgccgaaggattacgatgatgactacgcagatagtatccaagaagacggtgaaattgatggagattctggctgctatcgtgttctctcaaccaagactttagcagatgtggttgaggcacttattggggtctattatgtagcaggagggaaaattgctgcaaatcacctgatgaagtggattgggattcatgctgagttggatcctcacgagatccctcctccgaacccatataatatacctgagagcatattgagaagcatcaactttgacacattagaaggcatcttgggcatgaaattccagaatagaggttttcttattgaggctataacacatgcttcacggccatcttctggagtgtcctgttaccagcggttggaatttgttggtgatgcagtgttggatcatctcattacaaagcatttattttttacatatacagacctacctcctggccgcttgaccgacttgagagctgcagctgttaataatgagaacttcgcaagggtggcagtgaaacataaactgcatgtacaccttcgccatggatcatctgcactggaaacacagattcgggagtttgtgaaggatgtccaagaagagattgcaaaatcagatttcaattcttttgggcttggggactgcaaggctcctaaagttcttggagacattattgaatctattgctggcgctatattcctggatagtgggtataatacctcggcggtctggaaggtttttcaaccgctccttagtcctatggtgacgccagaaacccttcctatgcatccaattagggagctccaagaacggtgccagcagcaagccgaaggcttagaatacaaagcatcccgagcaggaaatgtagcaactgttgaagtgtttgttgatggtgttcagattggtctagctcagaacccgcagaagaagatggctcagaagttggcggcgaggaacgcacttgtggtcctgaaggaaaaggaaactgcaacgaaaaaggagaccgaacaggatggagataagaaaaacggtgctagtgtgttcaccaggcagacgctgaatgacatgtgcctgagaaggcagtggccgatgccacagtacaggtgcataaacgagggcggccctgctcatgctaagaagtttgtctacgcggtgagggtgaacacatccgatcgtggatggactgacgaatgcattgggcagccaatgccgagcgtgaagaaggccaaggactctgctgccgctcttctacttgagcttctgaatgaaagttgtaagaagtcatag

SEQ ID NO: 79 - DNA, 5211 bp, Lolium perenne
  misc_feature 1..5211: note = ORF of protein Dcl1
  source 1..5211: mol_type = unassigned DNA
atggcgggcggcggcggcgtcacgtactggtacgacgcgtgcgaggacgacgacgacgacgaggacagctcctcgctgctctgcggcatcgacttcgccgcgtccgctgacttcgacccggggttcttgcccaacttgggcctcgacagcggcggcgtcgacgacggcttcgtcgccgaaatcgaccggattctcgagagcatcaacgccgacaccgcccctgcgcccgcgcccgcgcccgcgccgccaccgcccccgcccccgtctcagcccgtggctacgccaccgccgcagccacagccgcagcaaaaggaggctcctttggccgtggcccacaatgccctagcggtggtggcccctgcctcggggcagggggtcgagccgaggaaggagacgcggagggagtcccacggcagctccgccaatggcggcggggattacaggggcgggaagcgccctcgcctcgtctccgggcctgacagggcgccgcgccccgacccgcggcggcgccccccgcccccgccctcgcgcgggtgggatgaccggcgcgggaggcgcgacttcgacaggcctccgcacaagcgcgaccgcgaccaccaccaccgccgcgaggcccgggggttctgggagcgggaccgcggcgggaagatggtgttccgccccggcgcctgggaccaggaggagtccggccgcgacgcgaagcgcgccagggcagagaacggaggcaccgtggggaagaaagcagaggcggctgctgcacagaaggagaagcccctcaccgaggagcatgcgaggcagtaccagctggaggtgctcgagcaggcaaagagcaggaacaccattgccttcctcgagaccggcgccgggaagacgctcattgccgtgctgctcgtcaagagtttatgcgacaagatgctcaaggagaacaagaagatacttgctgtgttcttggttcccaaggtgcctcttgtgtaccagcaagctgaggtgatccgtgacaggactggctatcgggttgggcattactgcggagagatgggtcaggatttttgggattctaggaagtggcagcgcgagtttgattctaaacaggtacttgttatgacagctcaaattttgttaaacatcctccggcacagtattattaaaatggatgcgatacatcttctaattttagacgagtgccatcatgcagtgaaaaaacatccatattctctagtgatgtcagaattttatcatacaactcccaaggataagagaccagttgtttttggtatgacagcttctcctgtcaacctgaagggagtcaccagccaagaggactgtgccatcaagataaggaatcttgaaactaaattagattccatagtttgtactatcaaagatcggaaggagcttgagaagcatgttcccatgcctttggaagtcgtcgtgcaatatgacaaagcagccactctttgttccttgcacgagaagatcaaacaaatggaaactgcagttgaggaagctgcactctctagctctaagagaagcaagtggcagttcatgggagccagggatgctgggtctagagatgagctgcgccttgtctatggtgtttctgagcgtacagaaagtgatggtgcagctaatttattccagaaattgagagctataaactatgctttaggtgaactaggacaatggtgtgcttacaaggttgcacagtccttcttgacagcgttacagaatgatgaaagggcaaactatcaggttgatgtaaagtttcaagagtcgtatttaaaaaaggttgttgatttattacactgccagttgactgaaggagctgctatgagaagcgagacaaatgatgttgagatgcataatgctgaaaacgacaatccaagtgagctagaggacggggagctgcctgatagccatgctgtttctgtaggtgaacatgtagatgaagtcatcggagccgccgtggcagatgggaaggttacccctcgggtgcaaagtttgatccaaatacttctcaagtatcagcataccaaggatttccgtgctatcatctttgtggagcgagttgtcaccgcattagttcttcctaaggtgttggcagagcttccatctctaagctttatacgatgtgcaagcttgataggccataacaacaatcaagagatgcgcacaagccaaatgcaggatacaattgcaaagtttcgggatggccgtgttacgctgcttgttgcgactagtgttgcagaggaaggtcttgatattcgacagtgcaatgttgtcatccgctttgaccttgcgaagactgtcttggcctacatacagtctcggggtcgtgcaagaaagcctggatctgattacatattgatggttgaaagggaaaatatgtcacatgaaactttcttgaggaattgtcgtaatagtgaggagacattgagaaaagaagctatagaaagaactgatctcactcatcttgatggcactcctgcgttaaatccaattgatacctcccctgattcgatgtaccaggtcgaatcaacaggtgctgttgttagcttgaattctgcagttggcctcgtacatttttactgctcacagcttccgagtgacaggtactcaattcttcgtccggaattttttatgcaaaggcacgagaagccagggggttcagtggaatattcttgcaaaattcaactgccctgtaatgctccatttgagaaacttgagggtccaatatgcagttccattcgtctggcacagcaagctgtttgtttggctgcgtgcaagaagctacatgagatgagtgccttcacagatatgcttttacctgatagaggaagtgaggaacgagaaaagactgaacagaatgatgaaggtgatccactccctggtacagcaaggcatagagagttttatcctgaaggagttgccgaaattctaagaggagaatggattttgtctggaaaaaatagctgccagagctcgcagtttattaagctgtacatgtattctgtgagctgcatagatgttgggacatctaaggaccctttccttgcacagctttcgaatttttctattattttcggcaatgagctggatgcagaggttttatcgacgacaattgatctctttgttgccaggacaatgataacaaaggcatctcttgtattccgtggaccaattgagattacagaaagtcagctggtcttgctgaagagctttcatgttaggctcatgagcatagtccttgatgtggatgttgatcccttaacgacaccttgggatccggcaaaagcatacctctttgtgcctgtaggagctgagaagtgtagtacagacgctttaagagagatcgattggaccttaattaacagcattgtgaacactgaagcttggaataacccacttcagaaggcacggccagatgtctacctaggaaccaaagagcggacacttggtggagataagaaagagtatgggttcggcaaattgcgtaatggcaccacttttggacagaaggctcatccgacttatggcataagaggagccattgcacagtttgatattgtcaaagcatctggattagttcctggtcgtgatagagggtattttaatgactatcagaatcaagggaagttgttcatggcagattcatgctgggatgctaaggatcttgctggaatggttgtgactgctgcccactcaggaaagaggttctatgtggactgtatctgttataacatgaatgcagaaaattcgtttcctaggaaggagggctacctggggcctttggagtatggttcgtatgctgactactacaagcagaagtatggcgtagagctagtgtataagaagcaacctcttatacgtgcacgaggtgtttcctattgcaagaatcttttgtctcccagatttgagcattcggaagctaaagaaggtgaccttccagagaaccttgacaagacgtactatgtgtatttaccacccgaactgtgctttgtacacccacttcctggatcacttgttcgtggagcccagaggttgccatcaataatgagaagggtggagagcatgcttcttgcagttcaattgaaggaaataattgactaccctgttcctgcaactaagatattagaagccttgactgctgcatcatgccaggaaacattctgctatgaaagagcagagttactgggtgatgcataccttaaatgggttgtgagtagattccttttcctaaaatatccccagaagcacgagggacagcttacaaggatgcggcagcagatgatcggtttgccccatcaagatgggcagctccgggagtgctacctgtatttgatgaggaaacaagagaatccgagccacctacctcctggccgcttgaccgacttgagagctgcagctgttaataatgagaacttcgcaagggtggcagtgaaacataaactgcatgtacaccttcgccatggatcatctgcactggaaacacagattcgggagtttgtgaaggatgtccaagaagagattgcaaaatcagatttcaattcttttgggcttggggactgcaaggctcctaaagttcttggagacattattgaatctattgctggcgctatattcctggatagtgggtataatacctcggcggtctggaaggtttttcaaccgctccttagtcctatggtgacgccagaaacccttcctatgcatccaattagggagctccaagaacggtgccagcagcaagccgaaggcttagaatacaaagcatcccgagcaggaaatgtagcaactgttgaagtgtttgttgatggtgttcagattggtctagctcagaacccgcagaagaagatggctcagaagttggcggcgaggaacgcacttgtggtcctgaaggaaaaggaaactgcaacgaaaaaggagaccgaacaggatggagataagaaaaacggtgctagtgtgttcaccaggcagacgctgaatgacatgtgcctgagaaggcagtggccgatgccacagtacaggtgcataaacgagggcggccctgctcatgctaagaagtttgtctacgcggtgagggtgaacacatccgatcgtggatggactgacgaatgcattgggcagccaatgccgagcgtgaagaaggccaaggactctgctgccgctcttctacttgagcttctgaatgaaagttgtaagaagtcatag

SEQ ID NO: 80 - DNA, 2115 bp, Arabidopsis thaliana
  misc_feature 1..2115: note = ORF of protein Dur3
  source 1..2115: mol_type = unassigned DNA
atggctacatgtcctcctttcgatttctcgaccaaatattacgatggtgacggcggatgccagaggcagagcagcttcttcggaggcacgacggtgctcgatcaaggcgttggctacgccgtgattcttggttttggtgctttctttgccgtcttcacatcttttttggtatggttagaaaaacgatatgtgggagctcgtcatacatcggaatggttcaatacagcgggaagaaacgttaagacaggacttattgcaagtgtcattgtatcacagtggacatgggctgcgaccatactgcaaagttcaaacgtagcatggcagtatggggttagtggaccgttttggtacgctagtggagccaccatacaggtgctgttgtttggtgtgatggcgattgagataaaaagaaaggctcctaatgctcacacggtctgcgaaatcgtgaaggctcgttggggaaccgcaacacatatagtgtttttggtgttttgtttggcgacaaacgtcgttgtgacggcgatgctcttgctcggcggctctgctgtggtgaatgctctaaccggagtgaatctatacgccgcaagctttctcattcctctcggcgtcgttgtctatactttggctggaggacttaaagctactttcctcgcaagctacgttcactccgttatagttcacgtggctctagtcgtctttgtgtttctggtctacacatcgagtaaagagcttggcagtccgtccgtggtctatgaccgactaaaagatatggtcgctaagtcaaggtcatgcactgagccactctcgcaccacggtcaagcttgtggtccggttgatggcaactttagaggctcctatctcaccatgttgagctctggcggcgctgtcttcggtctcatcaacattgtcggaaacttcggcactgtcttcgtagataatggttattgggtgagtgcaatagctgctcggccttcgtcgacccacaaagggtatttgcttggtggactcgtgtggttcgctgtaccgttctctctagctacctcgttaggtctcggcgcactagcccttgacttgcctatatctaaggacgaggctgaccgaggtttggttccacctgccacagccattgctctcatgggtaaatcaggatccttgcttctactcactatgctatttatggctgtaacatcggctggttcttccgagctaattgcagtctcatcgctctttacatatgacatctaccgaacatacataaaccctagagcaaccggtagacaaatcttgaaaatctcaagatgcgctgttcttggatttggttgtttcatggggattcttgcagtggtcttgaacaaagctggtgtttcacttggatggatgtatctcgcaatgggagtactcattggttctgcggtcattcccattgctttcatgctcttgtggagtaaagccaatgcctttggtgcaattcttggagcgacctcgggttgtgtttttgggatcattacttggctaactaccgcaaagacacagtacggacgcgttgatcttgatagcaccgggaaaaatggccctatgttagctggtaacctcgttgcgattctaacaggggggttgatccatgcggtgtgtagtctagtccggccacagaattacgattggtcgacgacgagggagatcaaagtagtggaagcgtatgcttcaggggacgaagatgttgatgttcctgccgaagagctaagagaagagaagctgagaagagccaaagcttggattgtgaaatggggtcttgtcttcactatcctcattgttgtcatttggccagttctctctctcccagctcgcgtatttagcagaggatatttctggttttgggcaattgtggcaatagcttggggaactataggatccatcgtgatcattggtttgccattggtggaaagttgggacacgatcaagagtgtttgtatgggaatgttcacaaacgatagggtgatgaaaaagcttgacgacttgaaccataggcttcgagctttaaccatggctgtgccagaagctgagaagatatatttgcttgaacttgagaagaccaagaaaaatgatgaagaaggttga

SEQ ID NO: 81 - DNA, 3885 bp, Arabidopsis thaliana
  misc_feature 1..3885: note = ORF of protein Ein2
  source 1..3885: mol_type = unassigned DNA
atggaagctgaaattgtgaatgtgagacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcgcataagcgttgtgactggtaaacacttggctcagatctgcaatgaagaatatgacaagtggacgtgcatgttcttgggcattcaggcggagttctcagcaattctgctcgaccttaccatggttgtgggagttgcgcatgcacttaaccttttgtttggggtggagttatccactggagtgtttttggccgccatggatgcgtttttatttcctgttttcgcctctttccttgaaaatggtatggcaaatacagtatccatttactctgcaggcctggtattacttctctatgtatctggcgtcttgctgagtcagtctgagatcccactctctatgaatggagtgttaactcggttaaatggagagagcgcattcgcactgatgggtcttcttggcgcaagcatcgtccctcacaatttttatatccattcttattttgctggggaaagtacatcttcgtctgatgtcgacaagagcagcttgtgtcaagaccatttgttcgccatctttggtgtcttcagcggactgtcacttgtaaattatgtattgatgaatgcagcagctaatgtgtttcacagtactggccttgtggtactgacttttcacgatgccttgtcactaatggagcaggtatttatgagtccgctcattccagtggtctttttgatgctcttgttcttctctagtcaaattaccgcactagcttgggctttcggtggagaggtcgtcctgcatgacttcctgaagatagaaatacccgcttggcttcatcgtgctacaatcagaattcttgcagttgctcctgcgctttattgtgtatggacatctggtgcagacggaatataccagttacttatattcacccaggtcttggtggcaatgatgcttccttgctcggtaataccgcttttccgcattgcttcgtcgagacaaatcatgggtgtccataaaatccctcaggttggcgagttcctcgcacttacaacgtttttgggatttctggggttgaatgttgtttttgttgttgagatggtatttgggagcagtgactgggctggtggtttgagatggaataccgtgatgggcacctcgattcagtacaccactctgcttgtatcgtcatgtgcatccttatgcctgatactctggctggcagccacgccgctgaaatctgcgagtaacagagcggaagctcaaatatggaacatggatgctcaaaatgctttatcttatccatctgttcaagaagaggaaattgaaagaacagaaacaaggaggaacgaagacgaatcaatagtgcggttggaaagcagggtaaaggatcagttggatactacgtctgttactagctcggtctatgatttgccagagaacattctaatgacggatcaagaaatccgttcgagccctccagaggaaagagagttggatgtaaagtactctacctctcaagttagtagtcttaaggaagactctgatgtaaaggaacagtctgtattgcagtcaacagtggttaatgaggtcagtgataaggatctgattgttgaaacaaagatggcgaaaattgaaccaatgagtcctgtggagaagattgttagcatggagaataacagcaagtttattgaaaaggatgttgaaggggtttcatgggaaacagaagaagctaccaaagctgctcctacaagcaactttactgtcggatctgatggtcctccttcattccgcagcttaagtggggaagggggaagtgggactggaagcctttcacggttgcaaggtttgggacgtgctgcccggagacacttatctgcgatccttgatgaattttggggacatttatatgattttcatgggcaattggttgctgaagccagggcaaagaaactagatcagctgtttggcactgatcaaaagtcagcctcttctatgaaagcagattcgtttggaaaagacattagcagtggatattgcatgtcaccaactgcgaagggaatggattcacagatgacttcaagtttatatgattcactgaagcagcagaggacaccgggaagtatcgattcgttgtatggattacaaagaggttcgtcaccgtcaccgttggtcaaccgtatgcagatgttgggtgcatatggtaacaccactaataataataatgcttacgaattgagtgagagaagatactctagcctgcgtgctccatcatcttcagagggttgggaacaccaacaaccagctacagttcacggataccagatgaagtcatatgtagacaatttggcaaaagaaaggcttgaagccttacaatcccgtggagagatcccgacatcgagatctatggcgcttggtacattgagctatacacagcaacttgctttagccttgaaacagaagtcccagaatggtctaacccctggaccagctcctgggtttgagaattttgctgggtctagaagcatatcgcgacaatctgaaagatcttattacggtgttccatcttctggcaatactgatactgttggcgcagcagtagccaatgagaaaaaatatagtagcatgccagatatctcaggattgtctatgtccgcaaggaacatgcatttaccaaacaacaagagtggatactgggatccgtcaagtggaggaggagggtatggtgcgtcttatggtcggttaagcaatgaatcatcgttatattctaatttggggtcacgggtgggagtaccctcgacttatgatgacatttctcaatcaagaggaggctacagagatgcctacagtttgccacagagtgcaacaacagggaccggatcgctttggtccagacagccctttgagcagtttggtgtagcggagaggaatggtgctgttggtgaggagctcaggaatagatcgaatccgatcaatatagacaacaacgcttcttctaatgttgatgcagaggctaagcttcttcagtcgttcaggcactgtattctaaagcttattaaacttgaaggatccgagtggttgtttggacaaagcgatggagttgatgaagaactgattgaccgggtagctgcacgagagaagtttatctatgaagctgaagctcgagaaataaaccaggtgggtcacatgggggagccactaatttcatcggttcctaactgtggagatggttgcgtttggagagctgatttgattgtgagctttggagtttggtgcattcaccgtgtccttgacttgtctctcatggagagtcggcctgagctttggggaaagtacacttacgttctcaaccgcctacagggagtgattgatccggcgttctcaaagctgcggacaccaatgacaccgtgcttttgccttcagattccagcgagccaccagagagcgagtccgacttcagctaacggaatgttacctccggctgcaaaaccggctaaaggcaaatgcacaaccgcagtcacacttcttgatctaatcaaagacgttgaaatggcaatctcttgtagaaaaggccgaaccggtacagctgcaggtgatgtggctttcccaaaggggaaagagaatttggcttcggttttgaagcggtataaacgtcggttatcgaataaaccagtaggtatgaatcaggatggacccggttcaagaaaaaacgtgactgcgtacggatcattgggttga

SEQ ID NO: 82 - DNA, 2691 bp, Arabidopsis thaliana
  misc_feature 1..2691: note = ORF of protein Emb175
  source 1..2691: mol_type = unassigned DNA
atgtccaccgttaatcatcactgcctcctcaattttcctcatatcccaccttcaattcctccgaatcatcgtcccaaacttctttcttctctctccctctatcgaaaaccagaacgcctctttgctctctctgcgtctctgtctctttcaccagcaacaatccatgaatgttcttcttcctcatcttcttcttcttcatcgttcgataaagaagaaacggaagacattgaatctgttattgatgggtttttctatctgcttcgattgtctgctcaataccacgacgtcgaggtaacaaaagctgtgcacgcttcgtttcttaagctcagagaagaaaaaacccgtcttggaaatgcccttatctctacttatctcaaacttggtttccctcgtgaagctattctcgtcttcgtgtctctgtcttcgccaacggtggtttcttacacagctcttatctcgggtttctcaaggttaaaccttgaaattgaggctcttaaggttttcttcaggatgagaaaagcaggtcttgttcagccgaatgagtatacttttgtagcgattttgactgcttgcgtgagagtttcccgattctcgcttgggattcagattcatggattgatagttaaatcagggtttttgaattctgtgtttgttagtaattcgctgatgagtttgtatgataaggattctgggtcttcgtgtgatgatgtgcttaagttgttcgatgaaattcctcagagagatgtggcgtcttggaacactgtggtttcgagtttagtgaaagagggcaagtctcataaagctttcgatttgttttatgagatgaatagagtcgaaggatttggagttgatagttttacgctttcaacgcttttgagctcttgtactgattctagtgtcttattgagaggtagagaactccatggtcgtgcaattaggatagggttgatgcaggagttgagtgtgaacaacgctctgattgggttttattctaaattttgggatatgaaaaaggtagagagtttatacgagatgatgatggcgcaagatgctgttactttcacggagatgatcacagcttacatgtcatttggaatggtggattccgctgttgagatattcgcgaatgtaactgaaaaaaacactattacgtataatgcgctcatggctgggttctgtagaaatggccatggcttgaaggcactaaaattattcactgatatgctgcagagaggtgttgagttgacggattttagtttaacaagcgctgttgatgcttgtggtttagtttcagagaagaaagtaagcgaacagattcacgggttttgtattaagtttggaactgctttcaacccttgtatccagactgcattgcttgatatgtgcacaaggtgtgaaaggatggcagatgctgaggagatgtttgaccaatggccttctaatctcgatagctcgaaagcaacgacgtctataatcggtggttacgctcggaatggcttacctgataaggctgtatcgctattccatcgcactctctgtgaacagaagttgtttttagatgaagtttcgttgactttgattcttgcggtctgcgggacattaggttttcgggaaatgggttatcagatccattgctatgctcttaaagctgggtacttttctgatataagcttgggaaattctttgattagcatgtacgcgaaatgttgtgactcagatgatgccataaagattttcaacactatgcgagaacatgatgttatttcttggaacagcttgatttcttgctacatccttcaaagaaatggtgatgaggcactggctctatggtcgaggatgaatgagaaagagatcaagcctgacataatcaccctcactcttgttatttctgcatttcggtatacagagtcaaataagcttagtagctgccgtgatctgtttctctccatgaagactatctatgacattgaaccaacgactgaacattacactgcattcgtccgtgttcttggtcattggggtcttcttgaagaagcggaagacacaataaactcaatgccggttcagcccgaggtctctgttttgagagctctgcttgatagctgcagaatacattcaaacacaagcgttgcgaaacgagtagcaaagcttatactatctacaaagccagaaaccccgtctgagtacatcctcaaatccaacatctactcagcttccggattctggcatcgctctgaaatgataagagaggaaatgcgggaaagaggttaccggaaacatcctgctaaaagctggataatccatgagaacaaaatacattcctttcacgcgcgtgacacatcccacccgcaagagaaagacatatacaggggattggagatactgataatggaatgtctgaaagttggatacgaaccaaacacagagtatgttcttcaggaggttgatgagtttatgaagaagagtttcttgtttcaccacagcgcgaaactcgcagtgacttatggaattctctcgagcaatactcggggcaagccggttcgggttatgaagaacgtaatgctctgtggtgattgccacgagttcttcaagtatatttcagttgtggtaaaaagagagatagttttgagagattcctcagggtttcatcattttgtgaatggcaagtgttcttgtagagatctgtggtga

SEQ ID NO: 83 - DNA, 2130 bp, Arabidopsis thaliana
  misc_feature 1..2130: note = ORF of protein Emb2726
  source 1..2130: mol_type = unassigned DNA
atggctacgattacaccttcttctattagcaatgcctggctcattcctggagcttcttttactgtaaagaagagtgattgttccatcaaatgtagcttttcgcgtaaagctgggaaacagattctatcatcaacgcagagacttgtgttacctttgtcgacttcacttagattgtttcctacacatggaaggcagtttgtgttgcatcctcatcgtagagctactgggactgatgttgtggcggctgtagaggaacaagattctactcctgttgttgcggaggataaagaaaccgtggctagtgagaagtcagatgctcctgctccaacatctcagtcaaggggaacagcaagacctggaaggaagagtgagatgcctgcggttaagaacgaggagctggttcctggagctactttcactgggaaagtacgagctattcagccttttggagcttttgttgattttggtgcttttactgatggattagtccatgtttctcaattgagtgacaactttgttaaggatgtttcgagtgttgtcactattggacaagaggtgaaggtaaggctagtggaagctgacattgagtcaaaacgtatttctttaactatgcgtgagaatgatgatcctcctaagcgtcagtctggtggtagtgataagccgagatcaggagggaaaagggatggttctaaaggaggaggacagaggaagggagaagggtttaactcaaagtttgccaaagggcagatgttggatggagtggtcaagaatttaacgaggtcaggagcgtttataactataggagaaggcgaagaagggtttttaccgacggctgaggaagcagatgatggtattggaagcatgatgatgggtggctcttcgttgcaagctggacaagaggtgaaggttcgtgtgttgaggatagcaaggggacgtgttactttaacaatgaaagaggaagatgatggtaagtttgatgaaacaaccactcagggagttgtgcatacagccacaaatccgtttatgcttgctttccgtaagaacgaagagattgctgcgtttcttgacaagagggaggaagaggcagagaagccacctgttgagacaccagtagagcctgaagctgaagcctctgtcacctcagctgaagtcgaggaatcggtatgtgtcccagctgaagtaacaagtgaagaggttccctcgtcagaaactcccaaggtagtagaagaagaagtgatagctaccaaagctgaagatgattcgccggaaaaagaagagcaaacggaaacacttgctgcagcagctgaagctgaagaggtggttcctccaattccagaaaccaaaagtgaagaggaaattgttgagaactctattcctccaaactcagccactgatgaagtttcatctccagaagctttagcaagtgaagaggttgagaaggagcaagtggttgccgaaacacctgttgatgaagtcaagactcctgcacctgtagtcacagaagcttcttctgaggaaagtggcaacaccgctactgctgaaagcatcaaaggcatttcaccagctctagtgaagcagctgagagaagaaacaggggcaggaatgatggactgcaagaatgctctttcagagagtgagggcgatatggtcaaagctcaagagtacctccgcaagaagggactagcgagcgcagataagaaagcaagcagagccacatccgagggaagaattggtgcttatatccacgacagcagaatcggtgtcctcttggaggttaattgtgagactgattttgtctcacgtggcgacattttcaaggaacttgttgatgatctggcgatgcaggtgcaatatgtttcaatagaggacattccagaggagataaagcagaaagaaaaagagattgagatgcaaagagaggatcttttgtcgaaaccagagaacataagggagaagattgtggaaggaagaatctcaaagaggcttggagaatgggcattgcttgagcagccttacatcaaagacgacagtgttctggtgaaggatctggtgaagcaaacagtggctactcttggggaaaacatcaaagtcagaaggtttgtgaagtttactcttggagaagataactga

SEQ ID NO: 84 - DNA, 1593 bp, Arabidopsis thaliana
  misc_feature 1..1593: note = ORF of protein Emb9
  source 1..1593: mol_type = unassigned DNA
atgaggacactctggaatcactttagtaccatttcctatatcaaaatttctccgagaatgagaagaatttctgcagcaaatttgatctcaaatcgaaatctttcaaccatttcttctactgaagatcccgagctcagagattttgtgggatttttagaatctctcaaaaactatgagaaatcaggtgtaccaaaaggagctggaactgattctgatgatggattcgatctgggtcgaatgaaacgtctcatgcttcgcctccgtaatcctcattacaaatacaaggttgttcatgttgctggaactaagggaaaaggatcaacttctgcttttctctctaatatcttacgagctggaggatattctgttggttgttattctagcccacatattctgagtatcaaagaacggatttcttgtaatggagaacctgtctctgcttccactcttaatgatcttttctattcagtcaaaccgattcttgaacagtctattcaagaggaaaatggttctttgagtcattttgagattctcactgggatagccttctctttatttgaaaaggaaaatgtcgacattgcggttatagaggctgggctaggaggagctcgagacgctacaaatgtcattgaaagttcaaatcttgctgcatcggtcataacaacgataggtgaggaacacatggcagcacttgggggttccttggaaagtatagcagaggctaaatctggaattattaaacacggtcgcccagtggttttaggtggaccgtttcttcctcatatcgagggcattctccgttctaaagcagcttcagtgtcgtcatcggttattttggcatctaatattggatctagttcttcaatcaaaggcatcattaacaaaaatgggatcgggctttgccagtcctgtgacatcgtgatacagaatgagaaagatgatcaaccaattgttgagcttagtgatgtaaacctacgtatgcttggacatcaccagctccaaaatgctgttactgccacatgtgtgtctctttgtcttcgcgatcaaggatgtgggagagtgacagatgaagcaattaggattggtttagagaacactcgtttacttgggagaagtcagtttctgacaccaaaagaagcagagactttactattacccggagcaacagtgcttcttgatggagctcacaccaaagagtcagcgcgagccctcaaggagatgataaagaaggattttccagagaaaagattggtatttgtggttgccatggctagtgacaaagatcatgtgtcctttgcaaaagaacttctctcaggtctaaaaccagaagcagtgattctaacagaagctgacatcggtggaggtaagattagatcaacggagtcttcggtgttgaaagaatcatggataaaagctgctgatgaattggggtcaaggtctatggaagcttcagaaaacaaaactgtgttgggttccttaaagcttgcctacaagatactcagcgatgacacaacaagtagtgactcaggaatggttatagtcacgggttcgcttcacattgtatcttcagtcttagcttctcttcaacattaa

SEQ ID NO: 85 - DNA, 1563 bp, Arabidopsis thaliana
  misc_feature 1..1563: note = ORF of protein Epsps
  source 1..1563: mol_type = unassigned DNA
atggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcggagaaagcgtcggagattgtacttcaacccattagagaaatctccggtcttattaagcttcctggctccaagtctctatcaaatcggatcctgcttctcgctgctctgtctgagggaacaactgtagtggacaacttgttgaatagcgatgacatcaattacatgcttgatgcgttgaagagattgggacttaatgtggaaactgacagtgaaaataatcgtgctgtagttgaaggatgtggcgggatattcccagcttccatagattcaaagagtgatatcgaactttacctcggtaatgcaggaacagcaatgcgtccacttaccgctgcggtcactgctgcaggtggaaacgcaagttatgtgcttgatggggtgcctcgtatgagagaaagacctataggggatttggttgttggtcttaagcagcttggtgctgatgttgaatgtactcttggaactaactgccctcctgttcgtgtcaacgctaatggtggccttcccggtggaaaggtgaagctttctggatcaattagtagtcagtacttgactgctctgctcatgtctgctcccttagctcttggagacgtcgagattgagattgtcgataaattaatttctgttccatatgttgaaatgacattgaagttgatggaacgtttcggggttagtgtcgagcatagtgatagctgggatcgtttctttgtcaagggcgggcaaaaatacaagtctccgggtaatgcgtatgtagaaggtgatgcttctagtgctagttatttcttggctggtgctgccattaccggtgaaactgtcacagtcgaaggttgtggaactaccagcttgcagggagatgtaaaattcgccgaggtccttgagaaaatgggatgtaaagtgtcctggacagagaacagtgtgactgtgacaggaccacctagagatgcttttggaatgagacacttgcgggctattgatgtcaacatgaacaaaatgcctgatgtagccatgacccttgccgtcgttgctctctttgctgacggtccaaccaccattagagatgtggctagctggagagtaaaggagacagaaaggatgattgccatttgcacagagcttagaaaactgggagctacagtggaagaaggttcagattattgtgtgataactccgcccaaaaaggtgaaaacggcagagattgatacatatgatgatcatagaatggcaatggcattctctcttgcagcttgtgctgatgttccaatcaccatcaacgatcctggttgcaccaggaaaaccttccccgactacttccaagtacttgaaagaatcacaaagcactaa

SEQ ID NO: 86 - DNA, 1083 bp, Arabidopsis thaliana
  misc_feature 1..1083: note = ORF of protein Fnr1
  source 1..1083: mol_type = unassigned DNA
atggctgctgctataagtgctgcagtctctttaccttcctccaagtcatcctctctcctcaccaaaatctcctctgtatcccctcaaaggattttcctcaagaagagcacagtgtgttacagaagagttgtgtcagtgaaggctcaggtgacaacagatactaccgaggcaccaccagttaaagtagtcaaggagtctaagaaacaggaagaagggattgttgtcaacaaattcaaacctaagaacccttacactggtcgctgccttttgaacaccaagatcaccggtgatgacgctcccggtgagacttggcacattgtcttcaccaccgaaggtgaggttccgtatagagaaggacaatcgataggagtgattccagagggaatagacaagaacgggaagccgcacaagctcaggctttactctatcgcgagtagtgccattggtgactttggagactccaagaccgtttctctctgtgtcaagagactagtttacacaaatgatggcggagagattgttaagggggtctgctccaacttcttgtgtgacttgaagccgggtgatgaagctaagatcactggacctgttggcaaggaaatgcttatgccaaaagaccccaatgccaccatcatcatgcttggaacaggaactggaatagctccattcagatcatttttgtggaaaatgttctttgaggagcacgaggactacaagttcaatggtttggcgtggcttttcttgggtgtacccacaagcagctcactgctatacaaggaggagtttgagaagatgaaggagaagaacccagacaacttcaggctggactttgcggtgagcagagagcagacgaacgagaagggagagaaaatgtacattcagacaagaatggcagagtatgcagaagagctgtgggagttgctgaagaaagacaacacctttgtttacatgtgtggtcttaagggtatggagaagggtatcgatgacattatggtctcgcttgctgctaaagatgggatcgattggttggagtacaagaagcaattgaagaggagtgaacagtggaatgttgaagtctactaa

SEQ ID NO: 87 - DNA, 1524 bp, Arabidopsis thaliana
  misc_feature 1..1524: note = ORF of protein Fve
  source 1..1524: mol_type = unassigned DNA
atggagagcgacgaagcagcagcagtgtctcctcaagcaacgacaccgagcggaggaaccggagcttctgggccgaagaagagaggtcggaaacctaaaaccaaggaagattctcagacgccgtcgtctcagcaacagagcgatgttaaaatgaaagaaagtgggaagaaaacgcagcagtcgccgagtgttgacgagaagtactctcagtggaaaggtctcgtccccattctctacgactggctcgctaaccataacctcgtctggccttcactctcttgcagatggggtccgcagcttgagcaagcaacctacaagaatcgccagcgtctgtacctctcagagcaaactgatggaagtgtgcccaatactttggtcatagcaaattgtgaagttgttaagccaagggttgctgcagcagagcacatttctcagttcaatgaagaagcacgttctccatttgtgaagaagtacaagaccatcattcaccctggagaggttaacagaatcagggaactcccacagaatagtaagattgttgctactcacaccgacagtcctgatgttctcatttgggatgttgaaacccaaccaaaccgtcatgctgtgcttggagctgcaaattcccgtccagatttgatactaactgggcaccaagataatgctgaatttgctcttgccatgtgcccaacggaaccctttgtgctctccggaggcaaggacaagtcagttgttttgtggagtatccaagatcacatcacaacgattgggacagattccaaatcatctggatctatcatcaaacagactggtgaaggtactgataagaatgagagtcctactgttggcccacgaggtgtatatcatggccatgaagatacagttgaagatgtggcattcagcccgacgagtgcacaagaattctgcagtgttggtgatgattcttgccttatactatgggatgcgagaactggcacaaaccctgtcacgaaggttgaaaaagcgcatgatgctgatcttcattgtgttgattggaatcctcatgacgacaatctgatcctgacagggtcagcagacaacactgtccggttgtttgatcgtaggaagcttaccgctaatggagttggttcgcctatttacaaatttgagggacacaaagctgctgttctttgtgttcagtggtctcctgataagtcatccgtctttgggagctctgcagaagatggtctcttgaacatctgggattatgacagggtcagtaagaagtctgatcgtgcagctaaaagccccgctgggctcttcttccagcatgctggtcacagggacaaagttgttgatttccactggaatgcttcagacccttggactattgtcagtgtttctgatgactgtgagactactggtggaggtggaacattgcagatatggcggatgagtgacttgatttacagaccagaagaagaagtcgtggcagaattggagaagttcaagtcgcatgttatgacttgtgcctccaagccttaa

SEQ ID NO: 88 - DNA, 1011 bp, Arabidopsis thaliana
  misc_feature 1..1011: note = ORF of protein Ga2ox7
  source 1..1011: mol_type = unassigned DNA
atggcttctcaacctccctttaagacaaatttctgctcgattttcggaagctcatttccaaattcaactagtgagagcaatacaaacacatcaactatccaaacctcaggcataaagcttcctgtgatcgatctcagccatctaactagtggtgaggaggtcaaacgcaaaagatgtgtgaaacaaatggttgcagctgcgaaagagtggggattttttcaaattgtgaaccatggaattcccaaagacgtctttgagatgatgctcctcgaagagaagaaactctttgaccaacctttttctgtgaaagtcagagaacgtttttcggacttatcgaagaatagttaccgttggggaaaccctagcgccacttctcccgctcagtactccgtttcggaagcgtttcacatcattctttcagaggtttcaaggatttctgatgatcgcaacaacctcagaacaatcgttgaaacgtatgtgcaagagatagctcgagtggcacaaatgatatgtgaaatactggggaaacaagtgaacgtgagttcggagtatttcgaaaacatttttgagcttgaaaacagttttctaaggctcaataagtaccatcctagtgtttttggttctgaagtgtttggtttggttcctcataccgatacaagttttctcactatactctctcaagatcaaatcggagggttagaattggaaaataatggacaatggatcagcgtaaaaccttgcttggaagcccttacagtcaacattggggatatgtttcaggcactgagtaatggagtgtaccaaagcgtgagacatagagtgatttctccagcaaatatcgagaggatgtcaatagctttcttcgtatgtccttatctcgaaactgagatcgattgctttgggtatccaaagaagtatagaagattcagtttcagagagtacaaagagcagagtgaacatgatgttaaagaaactggtgataaggtaggcttgtccaggtttctcatttga

SEQ ID NO: 89 - DNA, 1023 bp, Nicotiana benthamiana
  misc_feature 1..1023: note = ORF of protein Gapc
  source 1..1023: mol_type = unassigned DNA
atggcatctgacaagaagatcaagatcggaatcaatggatttggaaggattggtcgtttggtggctagagttgctctgcagagagatgatgttgaactagttgcagtgaacgacccatttatctctactggttacatgacatacatgtttaagtatgattcagttcatggacaatggaaacaccatgagcttaaagtcaaggatgaaaagacccttctttttggtgagaagtccgtcagagtctttggaatcaggaaccctgaagaaattccatgggctgaagctggtgctgatttcgttgtggaatccactggtgtcttcactgacaaggacaaggctgctgctcacttgaagggtggtgccaagaaggttgtgatctctgctcctagcaaggatgcccccatgtttgttgtgggtgtcaatgagaaggaatacaagccagaatatgacattgtctccaatgctagttgcactaccaactgccttgcacctttggctaaggtcatcaatgataggtttggcattgtggagggtctcatgacaactgtccactccctcactgccacccagaagactgttgatggtccatccatgaaggactggagaggtgggagagctgcttcattcaatatcattcctagcagcactggtgctgccaaggctgttggaaaagtgctcccagctcttaatggaaaattgacgggaatggccttcagagttccaactgttgatgtttctgttgtggaccttactgtcagactagagaaagaggcctcttatgatgacatcaaagctgcaatcaaggaggaatcagagggtaagttgaagggtatcttgggattcaccgaagatgatgtggtttccacagactttgttggtgacagcaggtcaagcatttttgatgccaaggctggaattgccttgagcaagaactttgtgaaacttgtgtcgtggtatgacaatgaatggggttacagctctcgtgtgattgatttgatctgccatatggcttctgttgcttga

SEQ ID NO: 90 - DNA, 2037 bp, Arabidopsis thaliana
  misc_feature 1..2037: note = ORF of protein Gcn2
  source 1..2037: mol_type = unassigned DNA
atggtattaacgacgaatctctatagcttaaaccttcgttcgaccttcttcttcactaatactattacttgccctacacttttcaccttcaaactctcctctgtttctaaccctagacgagtattccccaatattagagctcacgtttccgcagccagctccaattcagagctcgaatctctcttatctaccgatcggaagctgatcagtaagcaatcaaacaatggtgcgtcttcgatttcgtcgggagtgagacttgagaacattagtaagagctatgaaggcattacggtgctaaaggatgtgacttgggaagtgaagaaaggagaaaaagtagggttaatcggcgtcaacggtgcagggaagacgacacagctcaggatcataacgggtcaagaagaacctgattcaggaaacgtgatttgggccaaacccaatttgaaagttgctttcttgagtcaagagttcgaggtttcaatggggaagacagtgaaggaagagttcatgtgtacgtttaaggaagagatggaaatagcgaggaagcttgagaacttgcagaaggcgattgaagaagctgttgatgatttagaactaatgggaaagttactagacgaattcgatttgttgcagagacgagctcaagaggttgatttggattctatccatgctaaaatcagtaagctgatgtcggaattagggtttgtgtcggaggatgctgataggcttgtggcttcttttagcagtggatggcaaatgagaatgtcacttgggaagattttgcttcagaatccagatttactgcttttggatgagccaacgaatcatttggatcttgatactattgagtggcttgaaggttacttgattaagcaagatgtgcctatggtcataatatcacatgacagggcgtttcttgatcagttgtgtactaaaattgtggaaaccgaaatgggagtttcgaggactttcgatggtaattactctcagtatgtaatttcaaaggcagagttggttgaagctcagtatgctgcttgggagaagcaacagaaagagattgaagcaacaaaggatttgattagtaggctaagtgctggagcaaattctggtcgagcttcttcagcagaaaagaaactggagaagcttcaggaagaggagctaatagagaagccatttcagcggaaacaaatgaaaatccggtttccggaatgtggattaagtggaagatctgtagtcactgttaagaatcttgtatttggttttgatgataagatgttatttaacaaggcgaatctagctatagaaagaggagagaaagtcgctataattggtccaaacgggtgcggaaagagtacattgctgaaacttattatgggtttggagaagcctatgagaggtgaggtgatacttggggagcacaatgtattgccaaactacttcgagcagaatcaggcagaggctcaagatttggataaaacggtgattgagacagttgttgaagctgctgtagattggagaattgatgatataaaagctcttcttggtcggtgcaacttcaaagctgacatgttggatagaaaggtctctcttttgagtggtggtgagaaggcacgccttgctttctgcaaattcatggtgaaaccatccactctacttgtgttggacgaacccaccaatcacttagacataccttcaaaagaaatgcttgaggaagctataaatgagtacaaaggcacggtcatcacagtctcacacgatagatacttcattaaacagattgttaacagagttattgaagtcagagatggtggtttaatggactatgcaggagactacaattatttcctggagaagaatgttgaagctagagcaagggagctggagagggaagcagaattggaagaaaaagctccaaaagtgaaagcaaagtcaaagatgtcaaaggctgagagagaagcacggaagaagcaaaagatgaaagcgtttcaagcttccaaaaagaagtcaaagtccagcaaaaatgccaagagatggaattaa

SEQ ID NO: 91 - DNA, 1335 bp, Arabidopsis thaliana
  misc_feature 1..1335: note = ORF of protein Gdi2
  source 1..1335: mol_type = unassigned DNA
atggatgaagagtacgaggttattgttctcggcaccggtctcaaggagtgtatcctcagcggtctcctttccgtcgatggtgtcaaggtgcttcacatggacaggaatgactactatggtggagaatcaacttcccttaatctcaatcagctttggaagaagttcaggggagaagagaaggctcctgagcatttaggcgctagccgggattacaatgttgacatgatgcctaagtttatgatgggaaatggcaagcttgttcgtacccttattcatacagatgttacaaagtatttgtccttcaaagctgttgatggaagctatgttttcgtcaaaggaaaggttcaaaaggtgccagctactcctatggaggccctgaaatcttctctcatgggcatatttgagaaacgtcgagccggcaagtttttcagttttgttcaagaatacgatgagaaggacccaaaaacacacgatggaatggatttgaccagagttacaacaaaggaactgattgcgaaatatggtcttgatggcaacactattgactttattggtcacgcagtggcacttcacacgaatgaccaacatctcgaccaacccgcctttgatactgtaatgagaatgaagctctatgcggaatctcttgcacgtttccaaggaacatctccatatatttatcctctctatgggttgggagaactaccccaggcatttgcacgacttagtgctgtctatggtggcacatatatgttgaacaaacctgagtgcaaggtagagtttgacgagggaggtaaggttattggtgtaacatctgagggagagactgctaaatgcaaaaagattgtgtgtgacccttcatacctgccgaacaaggttaggaagattggcagggttgctcgggccatcgccattatgagccacccgattccgaacaccaatgattctcactcagtgcaggtcatcataccccagaaacagttggcccgcaaatcggatatgtatgtcttctgttgttcgtactcccacaacgttgctcccaagggaaaattcattgcatttgtgtctacagatgcagagactgataaccctcaaaccgaactaaagcctggaactgatcttttgggtcctgttgatgagatattcttcgacatgtatgatagatacgagcctgtcaacgagccagagttggacaactgctttatttcaacgagctatgatgctacaacacactttgagacaactgttgcggatgtgttgaacatgtataccctaatcaccggaaagcaactggacctaagtgttgacctgagtgccgcaagtgctgcagaggaatga

SEQ ID NO: 92 - DNA, 1293 bp, Arabidopsis thaliana
  misc_feature 1..1293: note = ORF of protein Gln2
  source 1..1293: mol_type = unassigned DNA
atggctcagatcttagcagcttctccaacatgtcagatgagagtgcctaaacactcatcagtcattgcatcatcatccaagttatggagctctgttgtgttgaaacagaagaagcagagcaacaacaaagtcagaggctttagagttcttgctctccaatctgataacagtactgtcaatagagttgagactcttctcaatttagacaccaaaccttactctgacaggatcattgctgaatacatttggatcggaggatctggaattgaccttagaagcaagtcaaggactatcgaaaagccggtggaggatccttctgagctacctaagtggaactatgatggttcgagtaccggtcaagcacctggtgaagatagtgaagtgattctatacccgcaagctatcttcagagatcctttccgtggaggcaataacatcttggttatctgtgatacttggacaccagctggtgagccaattccaacaaacaaacgtgctaaagctgctgagatcttcagtaacaagaaggtctctggcgaggttccatggttcggcattgaacaagagtacactttacttcagcaaaacgtcaaatggcctttaggttggcctgttggagcgttccctggtcctcagggtccttactactgtggagttggagctgacaagatttgggggcgtgacatttcagatgctcattacaaagcttgtttatatgctggaattaacattagtggtactaatggtgaagttatgcctggacagtgggagttccaagttggcccgagcgtaggaattgatgcaggtgatcatgtttggtgtgctagataccttcttgagagaatcacagaacaagctggtgttgtcctaacacttgatcccaaaccgatagagggtgactggaacggtgctggttgccacaccaattacagtaccaagagcatgagagaggaaggaggatttgaagtgatcaagaaggctatcttgaacctctcgcttcgccacaaggagcacatcagtgcctacggtgaaggaaacgagagaaggttgaccggaaagcacgagacagctagtattgaccagttctcatggggcgtggctaaccgtggatgctctattcgtgtgggacgtgacaccgaggcgaaaggaaaaggttacttagaagatcgccgtccagcatctaacatggacccatacattgtgacctcacttttggcagagaccacactcctgtgggagccaactcttgaggctgaagcccttgcagctcaaaagctttctttgaatgtttaa

SEQ ID NO: 93 - DNA, 5853 bp, Arabidopsis thaliana
  misc_feature 1..5853: note = ORF of protein Gsl3
  source 1..5853: mol_type = unassigned DNA
atggctcaaaggaaaggacctgatccgcctccgcctcaaaggcggattctgcggactcaaactgctggtaatctcggagaggctatgttggacagtgaagtggtgccatcgtctcttgtggagattgctccgatccttcgtgtagctaatgaagttgaagctagtaacccccgtgttgcttacttatgtcggttttatgcgtttgagaaggcacacaggcttgatcccacatcaagtggacgtggtgttcgccagtttaagactgcgcttcttcaacggttagaacgggagaatgaaacaactcttgcaggaaggcagaagagtgatgcacgcgaaatgcaaagtttctatcaacactactacaagaaatacatccaagctttgcaaaacgccgctgacaaagctgaccgtgctcaacttacaaaggcatatcaaactgctgctgttctctttgaagtcttgaaagctgttaatcagacagaagatgtggaagttgctgatgagattttggaggctcacacaaaggtggaagaaaaaagccaaatctacgtgccttacaacattcttccccttgatcctgatagtcaaaatcaggctataatgagatttcctgagatacaagctacagtaagcgcacttcgtaacactaggggtttaccatggccagctggtcacaagaagaaactcgatgaagacatgctagactggcttcaaactatgtttggttttcagaaagataatgtttcaaatcaaagagagcacttgattttgttacttgctaatgtccacattcgacagtttccaagacctgaacaacaaccaaggctggatgatcgtgcattgactatagtgatgaagaaacttttcaaaaactacaaaaagtggtgcaagtatttgggtcggaagagcagcctttggttgccgacgattcaacaagaggttcagcagcggaaactgctatacatgggactctatcttttaatatggggagaagctgcaaacctgagatttttgccagaatgcctttgctatatatatcaccatatggcttttgagctgtatgggatgttggctgggagtgttagtccaatgacaggggagcatgtaaaaccggcatatggtggtgaagacgaagctttcctgcaaaaagtagtgactcctatatacaagacaatagcaaaggaagcaaagagaagtagaggaggaaaatcaaagcattctgagtggagaaactatgacgacttgaatgaatatttttggtcaattcggtgctttcggttagggtggcctatgcgagctgatgctgatttcttttgtcagacagcggaagaacttcgacttgacagaagtgagaataaaccaaaaactggagatcgatggatgggcaaagtcaattttgttgagatacgctccttctggcatatatttagaagtttcgatagaatgtggagtttctatatcctgtctttgcaggcaatgatcataattgcttggaatggatctgggaaattaagcggcatcttccaaggtgatgtcttcctaaaggttttgagcatcttcattacggcagctatattaaagcttgcacaagctgtccttgatatagctctaagctggaaatcaagacatagtatgtcattccatgtgaagctcagatttatcttcaaggcagttgcagctgcaatctgggtggttctcatgccactaacttacgcatacagctggaagaccccttctggttttgcagagaccataaagaactggtttgggggacaccaaaattcctcaccttcctttttcattattgtcattcttatctacttatccccaaatatgctctctacattgctcttcgcgttcccattcatccgacgttatcttgaaaggtctgactataaaatagtgatgcttatgatgtggtggtctcagccgagactctatatagggaggggtatgcatgagagtgcattgtcacttttcaagtacacgatgttctgggttgtacttctaatatcaaagcttgcattcagtttttatgcagagattaagcctcttgttaaaccaaccaaagacattatgcgagttcatatatcagtctaccgctggcatgagtttttccctcacgccaagagtaatatgggtgtggtgatcgctctctggtcacctgttattcttgtttatttcatggacactcaaatatggtatgctatagtttcaactctggtgggaggtttaaatggagcttttcgacgtcttggagagatccgaactctaggaatgttgaggtctagatttcagtccttaccagaggcctttaatgcttgcttggttccaaatgagaagagcgaaactccaaagaaaaaggggattatggcgaccttcactcgcaagttcgatcaggttccatctagtaaagacaaggaggccgcacgatttgctcagatgtggaacaaaataatcagtagtttccgagaggaggatttgattagcgatagggaaatggaactcctgcttgtgccatattgggctgaccgtgatctggatcttatccgctggccaccttttctgctggctagtaagatcccaatagctttggatatggcaaaagatagcaatggaaaagatcgtgaactgaccaagagattgagtgtcgacagttacatgacttgtgcggttagagagtgctacgcctcttttaaaaatctcataaactttttggttgttggagaacgagaaggacaggtcataaacgaaatcttctccaggattgatgaacatatagagaaagaaacactaataaaggacttgaacttgagtgctcttcccgatctgtatggtcaatttgttcgactaattgaatatctgatggaaaacagagaggaggacaaggaccaaattgttattgtcttgctgaacatgttagaagtggtgacaagagacataatggacgaagaggtcccaagcatgctagagtcaactcacaatggaacttatgtcaagtacgacgtaatgactcctcttcatcaacagcgcaaatatttcagccaacttcgattcccagtgtactctcagacagaagcctggaaagaaaaaatcaagaggcttcatcttttacttactgtcaaagagtcggctatggatgtgccatccaacttggaagctcgaaggcgacttacattcttttcaaattcattattcatggaaatgcctgatgcccccaaaatccgcaacatgctttccttttcggtcctaacaccatactattcagaggatgttctattctccatttttggtttggagaagcaaaatgaagatggagtctctatactcttttacttgcagaagatctttccagatgagtggacaaactttttggaacgcgtgaaatgtggcagcgaagaagaactcagagcaagagaagagttggaagaggaacttcgtctgtgggcatcttacagaggccaaacactgaccaaaactgtgcgaggcatgatgtattaccgaaaggctttggagctccaggccttccttgatatggcaaaggatgaagaattaatgaaagggtacaaggctctcgagttaactagtgaagacgcatcgaagagtggaacgtcgctctgggctcagtgtcaagcacttgctgacatgaaatttacctttgtggtttcatgccaacaatacagtgttcagaagagatccggtgaccaacgtgcgaaggacatattgaggcttatgactacgtacccgtctctacgtgttgcttatattgacgaggttgagcaaacacataaagaaagctataaaggggcagacgaaaaaatttactactcagcccttgtcaaagctgctccgcagaccaaatctatggattcttctgaatctgtgcaaacactagaccaggtcatataccggattaagctaccaggacctgcaatactaggagaggggaagccagagaatcagaaccattctatcatttttactcgcggagaagggttacagacaatagacatgaaccaggacaattacatggaggaagctttcaaaatgaggaacttgcttcaagaatttcttgttaagcatggaggcgtaagaacccctacaatacttggacttagagagcatattttcacgggaagtgtttcttctcttgcatggtttatgtcgaatcaggaaaacagctttgttactattgggcagagggtgctagctagccccttgaaggtacgattccattacggtcatccagatgtttttgatcgactgtttcaccttaccagaggtggtgtctgcaaagcttctaaagttatcaatctaagtgaagacatatttgctggtttcaactctactcttcgtgaaggcaatgtgactcatcatgaatacatacaagttggtaaagggagagatgtaggactcaaccagatttcaatgtttgaggcaaagatcgctaacggaaatggggagcaaactctaagccgtgacctatataggctggggcaccgatttgatttcttccgcatgctttcttgttatttcactacaattgggttctacttcagtactatgttaaccgtgcttacggtctatgtatttctatatggacggctgtatctcgttcttagtggtcttgaagaaggattgagcaaccagaaagcttttcgatccaacatgcctcttcaagctgcacttgcttctcaatcatttgtgcagattggtttcttgatggccttacctatgatgatggagataggtcttgagaggggattccataacgccctaattgattttgtactgatgcagttacaactcgcttccgtgtttttcacgttccaactggggacaaaaactcattactatggtcgaacattgtttcatggcggcgcagaatacagaggcactgggcgtggttttgtggtgttccatgccaaatttgctgaaaactacagattctactcgcgcagtcattttgtgaagggtattgagctgatgatactcctgctagtgtatcagatatttggccatgcctatagaggagttgttacatatattctgatcactgtttcgatatggttcatggtggtgacttggctctttgctcctttcctgttcaatccctctggtttcgagtggcaaaagatcgtagatgattggacagattggaataaatggatatataaccgagggggaattggtgttcctccagaaaagagttgggagtcttggtgggagaaggaaataggacatcttcgacactcgggaaaacggggtatcatcctcgagattgttttggctttacgcttcttcatctttcagtacggactggtttatcaactcagcacctttaaacaggaaaatcaaagtttgtggatttatggagcttcatggttcgtgatcttattcattttacttattgtaaagggtctgggtgtgggaagacaacggttcagcacaaacttccagcttctcttcaggatcatcaagggttttgtgttcctcacatttcttggacttctcattactttcttagctcttcgttttttaacacccaaggacatcttcctttgtatgctcgctttcatgccaaccggttggggaatgttactcattgcacaagcgtgtaagcctctgattcaacggctaggattctggtcatcggttaggacgctggctcgaggctatgagattctcatgggtttacttttgttcactccggtagcgttcctggcttggttcccgttcgtgtctgagtttcagacaagaatgctctttaaccaagcatttagcagaggattgcagatttcacgtattcttggtggtcagagaaaagaccgctcttccaagaacaaggagtga

SEQ ID NO: 94 - DNA, 1338 bp, Arabidopsis thaliana
  misc_feature 1..1338: note = ORF of protein Hag5
  source 1..1338: mol_type = unassigned DNA
atgggatcgtcagcgaatacagaaaccaacggcaacgcaccgccaccgtcgtcgaatcaaaagcctccggctacgaacggcgttgatgggtctcatcctcctcctcctcctttaactcctgatcaagctattatagagtcggatccgtcgaagaagaggaaaatggggatgcttcctctagaagtgggtactcgtgtgatgtgtcggtggagagacgggaaacaccatccggtgaaagtaattgagcgccggcggatacataacggcggtcaaaatgattacgagtattacgttcattacactgagtttaataggaggctggatgaatggactcagctggaccaactggaccttgattcagtagagtgcgctgtagatgaaaaagtggaagacaaggtaacaagcttgaagatgacacgtcaccagaagaggaagatcgatgagacacatatagagggtcatgaagagctggatgcagcaagtttgcgtgaacatgaagagttcacgaaagtgaagaacatatcaacaattgagcttggaaaatatgagattgagacttggtacttctccccttttccgccagaatacaatgactgtgtgaagctctttttttgtgagttttgcctgaacttcatgaaacgcaaagagcagcttcaaaggcatatgaggaagtgtgacctgaagcacccacctggtgatgaaatttaccgaagtggtaccttgtcaatgtttgaggtagatggcaaaaagaacaaggtttatgcacagaatctctgctacctggcaaagttatttcttgaccacaaaactctttactacgatgttgatttgtttctattctacgttctttgcgaatgtgatgaccgaggatgccacatggttgggtacttttcaaaggagaagcattcggaagaagcatacaacttagcttgcattctaaccctgccttcatatcaaagaaaaggctatggaaagttcttaatagccttttcctatgaactgtcaaagaaagagggaaaagttgggacaccggaaagacccttgtcggatctaggcttactaagctacagaggttattggactcgtgttctattagaaatcttgaaaaaacataagggaaacatttctatcaaggagctgagcgacgtgacagcaatcaaagcggaagatatattaagcacacttcagagcctagaactgatacagtacaggaaaggacagcatgtgatctgtgcggatccaaaggttctggaccgacatctgaaagctgcaggccgaggtggtcttgatgtagatgctagcaaactgatttggacaccttacaaggaccagagttaa

SEQ ID NO: 95 - DNA, 2049 bp, Arabidopsis thaliana
  misc_feature 1..2049: note = ORF of protein Hda18
  source 1..2049: mol_type = unassigned DNA
atgcttttgaagtttgaagcttcttcagagttgagactagttgacccttcagtttctctcactgttcttcgaaagataagactgtcacatctcccggatatgacaatgaccagcgaatcttccggcaagaaatgcggagagggtgacggcaaagtcgccggtaaaagccagcgaaaggtgggtcttgtttatgacgagaccatgtgcaaacacgacacacctaacggtaaggttgacgtagagtgtcccgatcgtatcagagttatctgggagaagcttcagctcgccggagtcactcaaagatgtgtggttcttggtggtagtaaagctgaagataagcatctcaaattggttcacacaaagaaacatgttaatttagtgaagagtattagcacaaaaaagaaagattctcggagaaacaagattgcttcacagttggattcgatttatctgaatggaggttcatctgaagctgcatatcttgcagctggatctgttgtaaaggtggcagagaaagttgcagaaggagagttagattgtggctttgctattgtcaggcccccaggacaccatgccgagtcagatgaagccatgggtttttgtttattcaacaatgttgccgttgctgctagttttttactaaacgaaagacctgatctagatgtcaagaaaattctgattgtcgattgggatatccatcacggaaatggtacacagaagatgttctggaaagattctcgcgtactaatattctctgttcataggcatgatcacggaagtttctatccatttggcgatgatggagactttaacatggttggcgaaggtccaggtgaagggtttaacattaacgttccatgggagcaaggaggatgtggagatgcagactatcttgccgtatggaaccacatcttgatccctgtgaccaaggaattcaaaccagatattattttgctttcagctggatttgatgcagctattggtgatccgcttgggggttgttgtgttaccccatatggatattcagttatgttgaagaagctgatggagtttgcacatggaaagatagtgctggcattagagggaggatacaatcttgagtcattgggaaagtcttcactcgcatgtgtccaagttttgcttgaagacaaacaaattcatggttcatctgaaacatacccattagaatctacacggcgtgtcatacaagcggtacgcgagagactatgcacatattggccttcacttgacgcgtcaatggcgtctaatgagaatttaaagaacccaagtgcagaaagaaactccgctgacgccctccttcgtgaagtggaagagttaaagtctctaatggctgcccgagatggagaactggaggcaaggagaaaagagttaaaggccaaaaataaggagctagaagcgaacgagaaggagctagaagcgggactaatgttgattcgcgctcgggaggatgtaatctgtggtcttcatgcaaaaatagagagtttgcagcaggagcgagatgaggcggtagcgaaagctgaacgcatagataaggaactccaagaggatagggcaagaagccaggagttcaaagaagacacagagttttgcttgagtacgttaaggagggagaaggagcttgccatcatggccaaaaataaggacctagaagcgaaagagaaggagctagaagcgcggctaatgttggttcacgctcgggaggataaaattcatgccaaaatagagaggttgcagcaggagcgagatgaggccgtagcaaaagctgaacgcatagataaggaactccaagaggataggtcaagaagccgagttgggaatggatcttttgccttttctcaggagttctatgaagacatggacttagacgagctcgaacctctgtctccggagttcaatgaagacatggactcggaagagcttgaaccgtttcaggtcatcaagaaaaacatggagcgttcccacaagaagttcataaaagatatggagtgtatcaagtttatcgccagtgagagagccagggtactttag

SEQ ID NO: 96 - DNA, 1626 bp, Arabidopsis thaliana
  misc_feature 1..1626: note = ORF of protein Hexo1
  source 1..1626: mol_type = unassigned DNA
atgtcaacgaatcttcttcgtcttattctcctattcatcactttatctatcacttcatctctgtcaactccgtcgccggctgattctcctccttatctatggccgttaccggcggagttttcgttcggaaacgaaacgctctcggtggatcccaccgtgactctaatcgtagctggaaatggcggtggatctctgattattagagctgcgtttgataggtatatgggtataatctttaaacacgcttctggtagaggttctctattgtcgaggattagatttctgaaaatggttgagtatgatatcacttctttgaagatcgttgttcattctgattctgaggagcttcaattgggtgtggatgagagttatacattgatggtgtcgaagaagaacgagcaatcgattgttggagcagccacgatcgaggctaatacagtttatggtgcacttagaggattggagacatttagccagttgtgtgcctttgattatataaccaaatctgtgcaaatatataaagcaccatggtatattcaagataaacctagatttggataccgcggtctgctgatagatacatcaagacattatttaccaattgatgttattaagcaaataatcgaatccatgtcctttgccaaacttaatgtcctgcattggcacattgtagacgagcaatcatttcctcttgaaactcctacatatcctaatttgtggaaaggagcttattcgagatgggaacgttatacagttgaggatgcctctgagattgtcaggtttgccaaaatgagaggtattaatgtcatggctgaagtagatgtccctggccatgcggaatcgtggggaactggataccctgatctttggccgtctctgtcttgccgagagccccttgatgttactaaaaactttacattcgatgtgatttctggcattttagcagatatgagaaagatattcccatttgagcttttccacttgggtggtgatgaagttaatacagattgctggaaaaacacaactcatgttaaagaatggcttcagggtcgcaacttcacgactaaggacgcatataaatactttgtactccgagcgcaacagatagctatatcgaaaaattggacacctgtcaactgggaagaaaccttcagttcctttggaaaagatcttgatccgcggactgtgattcaaaactggctagtgtctgatatatgtcaaaaagcagtagcaaaaggattcaggtgcattttcagtaaccaaggatattggtacctcgaccatttagatgttccgtgggaagaggtttataacaccgagcctctaaatggaatcgaggatccttctctccaaaagcttgtcatcggaggagaagtttgcatgtggggtgaaacagctgatacatccgttgtccttcagaccatttggcctcgtgctgcagcagctgcagaacggatgtggagcaccagagaggccgtgtcaaaggggaacataacgttaaccgcattgccacgtctacactacttcagatgtctactgaataaccgtggcgttccagcggctccagtggataacttttacgcacgaagaccccccttgggacctggctcttgctatgctcagtga

SEQ ID NO: 97 - DNA, 1338 bp, Arabidopsis thaliana
  misc_feature 1..1338: note = (ORF of protein Hppd
  source 1..1338: mol_type = unassigned DNA
atgggccaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcttcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 98 - DNA, 2331 bp, Arabidopsis thaliana
  misc_feature 1..2331: note = ORF of protein Hsl1
  source 1..2331: mol_type = unassigned DNA
atggagtcaataaaggtttgcatgaacgcactgtgcggagcggcctctacgtcgggcgagtggaaaaaaggctggcctatgcgatccggtgatttagcttctctctgcgataagtgtgggtgtgcatacgagcaatccatattctgtgaagtgttccatgccaaggaatctggttggagagagtgtaattcatgtgacaagcgtcttcactgtggatgcattgcttctagatttatgatggagcttctagagaatggtggtgttacctgtataagttgcgccaagaaatccggactaatttctatgaatgtgagccatgaatctaacggtaaggacttcccctcatttgcttcagcagagcatgtaggcagtgttcttgagaggacaaatctcaagcacttgcttcactttcaaagaatcgaccccactcattcttctcttcaaatgaaacaagaagaatcgctgcttccttccagcctagatgctcttagacacaaaactgaaaggaaagaattgtctgcacagccaaacttgagcatttcacttggacctacgcttatgacaagcccatttcatgatgctgctgttgatgacagaagtaagactaattcgattttccaactggcccctcggtccaggcagctgcttccaaaacctgcaaattcagctcccattgctgctggcatggagcctagtgggagcctggtgtcacagattcatgtcgctcggcctcctccagaaggtcgcgggaagacccaattgcttccccgttactggcctaggattactgaccaagagctgctgcaattatctggacactcaaattccaaaattataccactctttgaaaaagttctgagtgcgagcgatgcgggtcgtattggtcgactggttcttccgaaagcatgtgcagaggcatatttcccccctatatctctacccgagggtctcccgttaaagatacaagacataaaagggaaagaatgggtgttccagttcaggttttggcctaataataacagcaggatgtacgttttggagggtgtgactccttgcatacagtccatgcagttgcaagctggtgacactgtaacattcagccgtacagaacctgaaggaaaactcgtaatgggataccgtaaagcgacgaactctacagcgacacagatgttcaagggaagcagtgaacccaatctgaacatgttttccaacagcttgaatccgggatgtggtgacatcaattggtctaaactagagaagtctgaggacatggcaaaggataacttatttcttcagtcgtccttaacttctgctaggaaacgggttcggaacattgggactaagagcaagcgtctgctcattgatagcgtagatgttctggaactgaaaataacttgggaggaggcacaggagctgttgcggcctccccaatccaccaaacccagcatctttacgctggaaaatcaagattttgaagaatatgacgaaccaccagttttcgggaagaggaccctttttgtctcacgtcaaacaggggaacaagagcaatgggtgcagtgtgatgcttgtgggaaatggcgacagctgccggtggatattcttcttccaccaaagtggtcgtgctctgataatctcttggatcctggcaggtcttcatgttccgcacctgatgaactctctccaagagaacaggatacacttgtccggcagagcaaagagttcaaaaggaggagactggcatcatcaaacgaaaagctaaaccagtcgcaggatgcatctgctctgaatagtttaggaaatgcaggcatcaccacaaccggtgaacagggggaaatcacggttgcagccacgaccaagcatccaagacaccgggcagggtgttcgtgcatcgtctgcagccaaccaccgagcggaaaaggcaaacacaagccgtcatgcacttgcactgtgtgcgaggcagtgaagagacgattcaggacgctcatgctgcggaagcggaacaaaggagaggcaggacaggcaagccagcaggcgcagtcacagtcagagtgcagggacgagacagaagtggagagcattccagcggttgaactagccgcaggggaaaacatcgacttgaactcagacccgggggcttcccgagtaagcatgatgaggcttctccaagctgcagcgtttcctctggaagcatatctgaaacaaaaggctatttccaatacagcaggagaacagcaaagcagtgatatggtcagcacagaacacggttcgtcctcagccgcacaagaaactgagaaagacacaacaaatggagctcatgatcctgtgaactaa

SEQ ID NO: 99 - DNA, 477 bp, Arabidopsis thaliana
  misc_feature 1..477: note = ORF of protein Iaa31
  source 1..477: mol_type = unassigned DNA
atggaggtctctaactcttgttcttcattttcttcatcctctgtcgacagtactaaaccttctccttctgaatcttctgttaatctctcccttagtctcacatttccttctacttctccacaaagagaagcaagacaagattggccaccgataaagtctagattaagagatacactaaagggtcgtcgtcttcttcgtcgtggtgatgacacttctctctttgttaaggtttatatggaaggtgttcccattggaagaaaactcgacctttgcgtattctcaggctacgagagtctattagaaaatctctctcacatgttcgatacttcaatcatctgcggtaatcgagatcgaaaacatcatgttttgacatatgaagacaaggatggagattggatgatggtcggagatattccatgggatatgtttcttgaaaccgtgagaagactaaagatcacgagaccggagaggtattaa

SEQ ID NO: 100 - DNA, 1995 bp, Arabidopsis thaliana
  misc_feature 1..1995: note = ORF of protein Iqd28
  source 1..1995: mol_type = unassigned DNA
atgggaaagactcctggtaaatggatcaagactttgcttctcgggaagaagtcgcctaagtcgaattcggataaccgaagccagaaactgaaatctgctaagaaagaagagctggtggaatcggtgacagaagatttgtcaaacttaacagttgatcctccggtggtttcatcacaaccagttccagcttctactgctcaaaatgtggtttctccaatcaatggtgatgagtccaaggataatctcgagtcaaggaatgacttgggagaagtcgagcttgaacaagctgcaattaaggttcaagctaccttcagagctcatcaggcacgtagagcatttcgaactcttaaaggtatcatcaggctgcaggcagttatccgtggtcacttggtgagaagacaagccattgctacatattcatgtatatggggaattgtgaagtttcaggctcttgttcgtggccagaaagctagatcttcagatattgcgattcagtttcagaaaaaacatatggaagcaagtgattcagaagttttgcaatcgagcacgtgtagttggatggataatcccacaaagttcgtctttgttgataagcttctagcttcttcaccaactgcattgcctctgaagatccagtatggtcctgaggaacctaactcagccaaggtttggcttgaacgctggacacaattacaagtttggtcttctggttcacgagtacctaggattgagattccaaaatctcagtcaaagaagcgaaactaccaagcagtggttgaagcagaaaagaaaaggccaaagcgaagtatcaagaaaccatctggcacaactagtggaactggccctagccgttttacagctgaaagaaataaacctaaacgaaatgtgaggaaggcttccacgcttagtaaagatcccctgagaaatgagagtgacaaagcaaatcacaattcgagaaaaagtagaagcggctcaaaggaggggtcgcctttggagattaaagatgagaagcccagtcctagtctcaaaaggtcttctctttcgaacgggtcaaagaaagccacccttagatctgctgagaagaagaagaaagatattccagattcttctgtgcaaatacagcctgaagggaaagtttcagagaatgttcttgaaggaggagacaatattgagtttgctgaaaaggagaaagatactacagattctgtgcagatagagtctgaagggaaggtttcagggaatgttcttgaaggaggagaaggagaaaatattgtgtttactgaaaaggagaaagatactgcagatcctgtgcagatagagcctgaaaggaaggttcttgaagaaggagacaatatcgagtcttctggaaaagaaaaagatactggagattctgtgcagatagagtctgaaggaaaggttcttgaaggaggagacaatattgaatttggtgaaaaagagaaagataaagctgatgcagttcccatcgaatttgatattgtaaaagatgagaagtcaccagttttggataggacggaggaagatgaactgaaaactgcagagaccagtgacaaagccgaagcccttaaatgtgcagatgtgaaggtaagctctgagaatggtaatgttggttctgataacacaaagcaaagtgaaaaacgagcattgctgcctgcaaatattgacaaacaagatgatgggcttacccttagcggacggaaaatccccagctacatggccccgactgcatctgcaaaggccagagtcaaaggagaagcctctccgaggtttgcccaagcgaagactgagataaatggggcattacggcgccattcactgccttctccagccaatggtaagctgagtactactactatgtctccaagggctcaaaaacttctcttagcctctgccaaaggatctatgaacggcgacaaatcttttacttcttccaaggacataactcacaagtcaaccagaaccgactggaaacggtga

SEQ ID NO: 101 - DNA, 1956 bp, Arabidopsis thaliana
  misc_feature 1..1956: note = ORF of protein Jac1
  source 1..1956: mol_type = unassigned DNA
atgcagacattaccaagctcagaaaccgttcttttaggctccaactcagcaccacctgtgttgcgtagtcccggtggcgatgacgtggacatcgattttggtgacgtgtttggtggtcctcctaagagacgctctaaagttactagtaacgaagtcacacgacatagcttcagtgaatccgctctccggcgtcgtgacgtcatcgtcgacgtcggcgatctgcttccgcaagacgagaagcctgtattcggcgaagatacatcatctgtccggcgtcgtttcaccactgatgatttcttcgacgatattttcagagttaatgaatcatcatcacttccgggttctcggatccttagcccggctcacaaacccgaatcttcttcagggacttcgtctccatcccaattcagtcttcctgcaaaagcaacagagattccaacatttaatttagcagctactcgtagtttgaacaagaacaaagaaactgtttcgagctctcctttgtcaagaacttcaagcaaagcagatgtggtctctactgctaaatcatattcagatgattgtgatgatcctccacaagtttttgttactgggaaaggaaggcaatttcatttctctatctacaagtggcctaataaaggagttcctgttgttatttggggaagctctagattgagttctatgtctaaagctgaggagacaacacctgtaccattgagtgattatcgaaaaacttcagttgttgagaaattgggtaaaaatgaagaaggagatggaaaaagtggtttatctggtttgaaagatgtgaagaagacctcgctaaagcgtcctggtgtccagacaaaagaagagaaaacagaaacagatttgaagtctgaacaagccttctttggtgtctcaaaagcacgtgaggctaatgtgaagcctcttgattcagttgagtctgaacaggccttctctggtgtgtcaaaagcacatgaagctactactgtgaagcctcttcattcaatttttcatgaagaagatgagagacaagatgagaaaatagtatcagagagagaagtgagaaaaggtaaaagcaaagcgaagaacactcgaagttttaccgaagattcaagaaccaagaagaaatcacaaggcactaaaagttctttggatagtagcccgatacccgataaatccagcttcgcaagttcatcagcagcaccagaagttggcaaagatggagtcaaaggaaaagtgagtgacttcgttaagattttcagtaaaggagcttcagttggagcaggtggggaatctcttggacaaagctctagatggagagctaaagaaactcccaagactgatatcattcatgatggttctaatgccaaggaaactgtaaacattcctgatcaacaaaagaaatcgactccagatatccccgctatgaatcgggatcagaaaccttcacagtcaactcagaaaaaagattcagaccgggagagcatgaactataaggctcctggtgatactgtacaagaagagagacaagaacctagcacaacacatactacctcagaggatatagatgagcccttccatgtaaatttcgatgtggaggatataacacaagatgaaaacaaaatggaagaggccaataaggatgctgaagaaatcaagaacatcgatgctaaaatccgcaagtggtcaagcggaaagagtggaaacattcggtctcttctatccacgctgcaatatattctttggtctgggagcgggtggaagccggttcctctaatggatatgatcgaaggaaacgcggttcgaaaatcgtaccagagggcattactaatacttcatccagacaaactacaacaaaagggtgcttctgcaaaccagaaatacatggccgagaaagttttcgaattattacaggaagcatgggaccacttcaacactctcggaccggtttaa

SEQ ID NO: 102 - DNA, 1728 bp, Arabidopsis thaliana
  misc_feature 1..1728: note = ORF of protein Jar1
  source 1..1728: mol_type = unassigned DNA
atgttggagaaggttgaaactttcgacatgaacagagtcattgatgaattcgatgaaatgactagaaacgctcatcaagtccagaaacaaactttgaaagagattcttctcaagaaccaatccgccatttacttacaaaactgtggtctcaatggaaacgctactgaccctgaagaagctttcaaatctatggttcctttagtcactgatgttgaattagagccttacatcaaaagaatggttgatggtgacacttcacctattctcactggtcaccctgttcctgccatttccttaagctctggaactagtcaaggccgtccaaagtttattcctttcactgatgaattaatggagaacacattgcaactgtttcgcactgcttttgccttcagaaacagagattttcccattgatgacaatggcaaagctttgcaatttatcttcagcagcaagcagtacatatcaacgggaggtgtcccggttggaaccgcaaccacaaacgtgtaccgcaaccctaacttcaaagctgggatgaaatctataacgtctccttcctgtagtcccgacgaagttatctttagtcctgatgtccatcaagccttgtattgccatctcttatccggaattctcttcagagatcaagttcaatacgtctttgccgtctttgctcacggtctagttcacgcttttagaacctttgaacaggtttgggaagagattgttaccgatatcaaagatggtgtcttaagcaaccgtattactgtcccatcggtccgtactgcaatgtcgaagctgctcacacctaaccctgagctagctgagacgatccgcaccaaatgtatgagtttgagcaactggtacggattgattcccgcgctcttccccaacgcaaagtatgtttacgggatcatgactggctccatggagccgtatgtgccaaagctgagacattacgcgggtgatctacctcttgtgagccatgactacggtagctcggaaggatggattgctgctaatgttaccccaaggttatctccagaggaagctacatttgctgtgattccgaatctcggttatttcgagtttctccctgtatctgaaacaggggaaggagaggagaaaccggttggtttaactcaagtcaagattggagaagagtatgaagttgtcatcacaaattacgcagggttgtatcgataccggcttggagatgtggtaaaggtcattggtttctacaacaacactccccaactaaaattcatatgcaggagaaacctgattctctcgatcaacatcgacaagaacaccgaaagagaccttcagctttcagtagaatcggctgcaaagagactctcagaagaaaagatcgaggtcatagacttctctagctacatcgatgtctcgacagatccaggacattacgctatcttctgggagatttcaggagaaacaaacgaagacgttcttcaagattgctgcaactgtttagaccgagcattcatcgatgcgggttacgttagttcccggaaatgcaagactattggagccttggagctgagagttgtcgcaaaaggaactttccggaagattcaagaacattttctcgggcttggttcatcggctggacagtttaagatgccaagatgtgtgaagccaagtaatgctaaggttctccaaattctgtgtgagaatgtggtgagtagctacttcagcacagcgttttga

SEQ ID NO: 103 - DNA, 3399 bp, Arabidopsis thaliana
  misc_feature 1..3399: note = ORF of protein Kp1
  source 1..3399: mol_type = unassigned DNA
atgccacaagaattaatgagaggattaaagggtttggtggtgtcgtccacaagcaatggagattatgagcttgctcaacgaaaagctgaagaaacagctctgagaagataccaagcggtggcatggctgagacagatggaccaaggcgcgatggagacactcccggaaaaaccgtcggaagacgagttctccctcgccctccgcaacggcctcattctctgcaacgtcctcaacaaagtcaatcccggttctgttctcaaggtggtggagaatccgattacgccagctattcagtacgcggatggagcggcgcaatcggcaattcaatattttgagaacatgaggaactttcttaaagctgtagaggatatgcagcttttaacatttggagcttctgatctcgagaagggaggatcgtcgaacaaagtagtagattgtattttgtgtttgaaaggtttttacgagtggaaacaagcgggtggagttggagtatggaggtatggagggactgtgaggatagtatcattcaatcgaaaaggcagttctccgccgcagtatgggattggtagtgaaagtactaccgatgagtctgtttctttggatgaatctgagtcttctcagtatgaccagcttttggattttcttcatctttctaatgagatctcagctgaggaatctgaaactgcaatctccttggcttttctttttgatcatttcgcgcttcagcttctacatggttaccttaaggagagtgatgggatcaatgatatgcctttaaatgaaatggtgattgatacattgctgaacagggtagttaaggatttctctgcaatactagtttctcagggagctcagctgggttcgtttctaaggaaaatactgaaatgtgataacggggatctgtcaagatcagagtttctagcagcagtcttcagataccttcagcacagaaaagatttggtgtcaaaggagttttcaaagttttgtaaatgtggcggaaagcttgagtttagtagactaaacgcccgcgaattttctccgggtcatgtagaagctattggtcttcaacagaaagagctggaggaagttaaatcaaattttgtggaaacaagaagccaagttaaacaaatgcagtcagaatggcagaaagagcttcagaggatagtccatcacgttaaggccatggaagttacatcttcttcttaccacaaggttctggaggaaaatcgcttactttacaatgaggttcaagatctcaaaggcaccattagagtttactgcagagtcagacctttcttccaagaacaaaaagatatgcaatcaactgtggattatattggagaaaatggtaatattatcattaacaatcctttcaagcaggagaaagatgcacggaaaatattttccttcaacaaagtatttggacagactgtctcacaagagcaaatctacattgacactcaaccggtaataaggtccgtcctcgacggattcaatgtttgtatcttcgcgtatgggcaaaccggttcagggaaaacgtatacgatgagtgggccagatctgatgacggagacaacttggggcgtgaactaccgagctctacgcgatctcttccagctttcaaatgcaagaacacatgtggtgacttatgaaattggagtccaaatgatcgaaatatataatgaacaagttagagatctactagtcagtgatggttcctccagaagattagacatacgcaataattctcaactcaatggccttaatgtacccgatgcaaacttgattccagtttctaatactcgagatgtccttgatttgatgaggattggccaaaagaatcgtgctgtgggtgccactgctttgaatgagaggagcagccgttctcatagtgtattaactgttcacgtccaaggaaaagaattggcctcaggatccattttaagaggctgccttcatttagtggatttggctggaagtgaaagagtagaaaaatccgaagctgtaggcgagagactcaaagaagctcaacacataaacaaatcgttgtctgcattaggagatgtcatttatgcacttgcacaaaagagttcccacgttccctacagaaatagcaagcttacacaagttctgcaggactccttaggtggacaagcaaaaactttaatgtttgtacatattaatcctgaagttaatgcggtaggggagactatcagcacccttaagtttgcccaaagggttgcatccattgagcttggagcagctcgatcaaacaaggaaaccggtgaaattcgagatcttaaggatgagatatccagccttaaatcggccatggagaagaaggaagcagagctggaacaattgcgatcaggtagcattcggaacacaactgaatgtcagagagcaagagcagtttctcctttccatctcccaagaactggtaacggtgctggaacaaaggctgaggcaagtccacagccaaacgacggtaccagaagctacgagaccagaagttgctcaactggcaagcagagaaagtcagggtttccgtctgcactgagaaatagagaagcaagcccgaggatgccgaatctagcagaagagaggttaaatccgagtccaagtaggagatcactatccacagacagagcatcaaccatcaaaagtaggaataagcccgacgttacccaaaaccttccggtttcaagaacaccttttccagctagagtaccagtagtcaaatccttttcaactgttccattgaacccatcagcagaaaataaccacaggcttcacactgataactcttcagaggcatttcaaaatcatcagaagttaagcgctcgaaagctcttccctgagatcgaggaggaacacataaggcatgcactacacatacgtcaaggtggtgttaagaagaccagagccgagagcagtaaagccaaggcaaagcaaccatcaccagctaggtttcagaaactcgatgttgggatcagtttgcgttctgatgcagattctgaagcaaaggtaggaaattatcagacccaaaaggggaacaacaaccacaatgtgatacactcaagattccaaaacttcgacgtgggtatcagcctattctctgacctttgtgccggtgacaaatcagattcaacactcaagagtgattcctcagagacagacaacgaaccaccctcgaaatctaagaatgcccaaagaaactcgtccaaaaactccctcaaccacaaactaaggacgatctatgctcatgaggacacttcactcgtggacgacaagccttctaatggtacagcccatatcaaagaaggtaataataatatatctatgcctgagttcaggagaagtcgttcaacacatcatgcaaggttcatggtaccatag

SEQ ID NO: 104 - DNA, 2361 bp, Arabidopsis thaliana
  misc_feature 1..2361: note = ORF of protein Lrx2
  source 1..2361: mol_type = unassigned DNA
atgttgttgtttccttctacttctttacgtctcttcttctttctcttcctcttgttctcctcttgtttcttacagattagaggagatgatgatgatgatgatataagcgatgataatatcaaagtcgacccgagtctcaagttcgaaaacccaagtcttcgacaagcctacattgctcttcaatcatggaaacaagctattttctctgatccttttaacttcactgctaattggaatggctctgatgtttgttcctacaacggtatcttttgtgctccttctccttctagtccgaaaactcgagttgttgcgggtattgaccttaaccatgcggacatggctggttatttacctcgtgagcttggtcttctcactgatcttgctctctttcatctcaactcaaaccgcttttgtggagaagtacctctcacgtttaaacacatgaagcttctcttcgagcttgacttaagtaacaaccgttttgttgggaagttccctaatgttgtcttgtctttaccttccctcaagttcttggatcttcggtacaacgagtttgaaggtagtatcccttcgaagctattcgataaagagctcgacgcgatattcttaaaccataaccggtttatgtttgggatacccgaaaacatgggaaactctccggtgtctgctctagttcttgcggataatgatcttggaggttgcataccgggaagtatcggtttaatgggaaagactctcaacgagatcatcctctctaacgataacttaaccggttgcttaccaccacagattgggaatctcaagaatgtgacggttttcgatatcagtttcaaccgtttaagcggtccattgccatcaagcattggaaacatgaagagtttggaacagctcaatgttgctaacaatagattcacaggagttataccaagcagcatttgtcagctctctaaccttgagaacttcacttattcttccaacttcttcaccggtgatgccccgagatgtgtggctcttttgggagacaatgtggtggttaatggctcgatgaattgtattgatggtaaagaagatcaaagatcgtctaaagagtgttcttctccagcctcacgctctgttgattgtagtaagtttgggtgtaacaactttttctctcctccaccaccgtcttttaagatgtcacccaccgttcgagtacttcctccaccgcctccgtcttccaagatgtcgcctacatttagagcaactccgccaccgccgtcttctaagatgtcaccatcttttagagcaactccgccaccgccttcttctaaaatgtcgccttcttttagagcaactccgccaccaccttcttctaagatgtcgccttccgtcaaggcgtatcctcctcctccaccaccgccagagtatgagccatctccgcctcctccttcttccgaaatgtcaccttccgttagggcataccctccaccaccaccactatcaccaccaccaccatctcctcctccaccatacatctactcatctcctccaccaccgtcaccatctcctcctccgccatacatttactcatctcctccgcccgtagttaactgtccacccacaacacaaagtcccccacctccgaaatatgagcaaactccttcaccaagggaatactatccatcaccttcaccaccttactaccaatacacgtcttctccgccaccgcctacttactacgccactcaatctccaccgccaccaccaccgcctacttactacgctgttcaatctcccccgccaccaccaccggtatactacccacctgtaacagcaagtcctcctccaccaccggtctactatacaccggttatacaatctcctcctcctccaccggtttactattcacctgttacccaaagtcctccaccaccaccaccggtttactatcccccggtaacacaaagtccaccaccatcaccggtctactatccgccagttacccaaagtccacctcccccaccagtttattatcttccagtaactcaaagccctccaccgccatcaccagtctactatccaccggtagcaaaaagccctccaccaccatcaccagtctactatccaccggtaacacaaagcccacctccaccgtcaacaccagttgagtaccatccaccggcaagcccaaaccagtctccaccaccggagtatcaatcaccaccaccaaagggatgcaacgacagtccaagtaacgaccatcactaccaaacaccaacaccaccttctctgccgcctccatattacgaagacacacctcttccgcctatccgcggtgtctcttatgcgtctcctcctccaccatcaatcccatactactaa

SEQ ID NO: 105 - DNA, 1830 bp, Arabidopsis thaliana
  misc_feature 1..1830: note = ORF of protein Mapkkk3
  source 1..1830: mol_type = unassigned DNA
atgcctacttggtggggaagaaagtcctgcaagaacaaggacgataatcacagaggaatcatctccacagatagagatatcaagagctctgctgttgttgttgatcctcctctcactcctactcgtggtggtacacctcgttgcagtcgtgaattcgctggagcttcttctgctttctctggttttgactctgattctaccgagaagaaaggtcaccctcttcctcgtcctttgctctctcctgtttcgatccatcatcaagatcatgtaagcggatccacttctggatctacatctgtttctagcgttagctcatctggatcagctgatgatcagagtcaacttgttgcttctaggggtcgtggtgatgtgaaattcaatgtagcagcagcaccacgtagtcctgagagagtttctccaaaggcagctactattactactaggcccacgtctccacggcatcagcgtttgtctggagttgtgagcttagagtcttctacagggaggaatgatgatggaaggtcctcctctgagtgtcatcctttgcctcgaccacctacttctcctacaagcccttctgctgtgcatggttccaggattggaggaggctacgagacctctccttcggggttttccacgtggaaaaaagggaaatttcttgggagtggcacctttggtcaagtctatcttggtttcaacagtgagaaagggaaaatgtgtgctattaaagaggtcaaggtcatttctgacgatcaaacatcaaaagaatgtctgaagcaactaaatcaggagataaatttgcttaaccagctttgtcatccgaatattgtccagtattacggaagtgaactgagtgaagaaaccttgtccgtctacttggagtacgtgtcaggtggttcaatccataaactacttaaggattatggttctttcactgaacccgttatccaaaactacacgcggcagattcttgctgggcttgcctatttacatggacgaaatacagtacatagggacatcaaaggagcaaatatattagtggatccgaatggtgaaatcaagttggcagactttgggatggccaaacatgtaacagccttttctactatgctttcttttaaagggagtccttactggatggcacccgaggttgtgatgagccaaaatggctacactcatgcagtcgatatctggagtttgggttgtactattctggaaatggcaacatcaaagccaccttggagccagtttgaaggggttgctgcgattttcaaaatcggaaacagtaaagacaccccggaaatacctgatcacctttcaaatgatgcaaagaattttataaggctttgtctgcaacgaaatccgacagtacgtcctacagcttctcagcttctagaacacccttttctacgtaacacaacaagagtggctagtactagtttgcccaaagacttccccccacgttcctatgatggaaacttctcactgcagcctacaagggaaccctatccagggagactgagccatgataattatgcaaaacagccattgtctagaactataaagagcccgagcagagaaaacgtaagagctatcacatccttaccagtatctccatgttcaagccctttacgccaacttgggccagcatacaaaagttgtttcctgtcacctcctcacccgtcttacgcatttcctgggcaagacagtgggtacaacctagcagagtttgctgcaagccctttcaggatgaagaaagacgcaatgatggaaccatctagtttcaggactcaaacaccgaattcaccattgagatcaagactggtgtag

SEQ ID NO: 106 - DNA, 2151 bp, Arabidopsis thaliana
  misc_feature 1..2151: note = ORF of protein Mapkkk5
  source 1..2151: mol_type = unassigned DNA
atgcgttggcttccgcaaatctcgttctcgtctccttcttcttctccatcttcttctctaaaacccgtggcttcttactctgaatctccggatccagatcgtaatcaggatcgggatcggtttcatcgccgcttgtttcgttttaaccgtggcaggctcacccgtcagcggaagcttcgtcacttgacggatgacgatgttttgttgggagaacgtcgtgcttctacctcttcctccaccttcgattccggtttaactcgctctcccagcgctttcaccgccgttcctcgctctccttctgccgttcccttacccttacctctccccttaccggaggtcgccgggattcgaaatgccgcaaacgctagaggattggatgacagagatcgagatcccgagagacttatttctgatcgcacctcttctggtcctcctctcaccagcgtcaatggcggctttgcccgtgactcgaggaaagctacggagaattcgtcatatcaagattttagtccgagaaacagaaatggttattgggtgaatattccaaccatgagtgcaccaacgagtccatacatgagtcctgtgcctagtccacaaaggaagagtactggccacgatttgccttttttttatttgcctcctaaaagcaatcaagcttggtctgctccagatatgccacttgatacctctggtcttcctcctcctgcattttatgatattactgcctttagtaccgataattctcccatccatagtccacaacctcggagtccacggaaacagatcagaagcccacaacctagcagaccatcttcaccgttgcattcggttgatagctcagctccaccgcgagatagtgtttcctcacctttgcatccgaggttgtctactgatgttacgaatgggcgacgtgattgctgcaatgttcatcctttgcctctccctcctggagctacttgttcctcttcatcagctgcttctgttccgtccccgcaggctcctctcaaactggattcattcccaatgaattcccagtggaagaaagggaagctaataggtcgtggtacttttggaagtgtttacgttgcaagcaacagcgaaactggagcattgtgtgcgatgaaagaagttgagctatttcctgatgaccccaaatccgcagagtgtataaagcaattagagcaggagatcaaacttctaagtaaccttcaacatccaaacattgtgcagtattttggtagcgagacagtagaagatcgtttctttatatacctggaatatgttcacccgggttcaataaacaaatatatccgtgatcattgcggcaccatgacagaatctgttgttcgcaattttactcgtcacattttgtctgggctggcttatttgcacaataaaaagactgtacatagggatatcaaaggtgctaatctccttgttgatgcttctggggttgtcaagcttgctgatttcggcatggctaaacaccttactggacaaagagctgatctctcgttaaagggaagcccgtactggatggcaccagagctcatgcaagctgtgatgcaaaaagatagcaacccagatctggcttttgctgttgatatatggagtttaggatgtacaatcattgagatgttcactgggaagcctccttggagtgagtttgaaggggctgcagctatgttcaaggtcatgagagatagcccaccgatacctgaatcaatgtcacctgagggtaaagacttcctgagattatgcttccagagaaacccagctgagcgaccaaccgcatctatgttgctagaacaccggttcctaaagaactctttgcaaccaacctcaccaagcaacagtgatgtctctcaattatttaatgggatgaacataacggaaccaagcagtagaagggagaagccaaatttcaaactagaccaggtcccgcgagctagaaacatgacatcctcagagagtgaaagtgggcaacagcagcagcaacaacagtaccggtctcccgatctaacaggaaccgtgaaccgtctgtctcctcgttccactctggaggctatcccaagcccgtgtccttcccaacgacctaagcccagcagcagtgacaggagaagaacgggcgtcacttcagatcacctttga

SEQ ID NO: 107 - DNA, 2178 bp, Arabidopsis thaliana
  misc_feature 1..2178: note = ORF of protein Mfp2
  source 1..2178: mol_type = unassigned DNA
atggattcacgaaccaaggggaagacggtgatggaagttggtggcgatggcgttgccgtcataacactcatcaatcctcccgtcaattctctatccttcgacgtgctatacaatctcaaaagtaattacgaggaggccttgagcaggaatgatgttaaagctattgttattactggtgcaaaggggaggttctctggtggctttgatatatctggttttggtgaaatgcagaaggggaatgtcaaagagccaaaggctggttacatttcgattgacatcataaccgacttgcttgaagctgctaggaaaccgtctgtcgctgccattgatggacttgccttgggaggaggattagaacttgctatggcttgtcatgctaggatatcagctcctgctgcacagttaggcttgcctgagctgcaacttggtgttattcctggttttggaggaacgcagcgtctcccgcgtcttgttggtctcaccaaagcccttgaaatgattttgacatctaagccagttaaagctgaggaaggtcattctttgggtcttattgatgctgtggtgccacctgcggagttagtaaccactgctcgtcgctgggcccttgacatagtcgggaggagaaaaccatgggtatctagtgtttccaagactgataagttacctcctcttggagaggcaagggagatactgacgtttgccaaggcacagacactcaagcgtgcccccaatatgaaacaccctttaatgtgccttgacgctattgaagtaggtattgtttctggtccaagggctggtttagaaaaggaagctgaagtcgcctcacaagtggtaaaactggataccaccaaaggcttgatccacgtcttcttttctcagcgaggaactgctaaggttcctggagttactgatcgtgggttggtgccaaggaagattaagaaggtagccataattggaggcgggttgatgggatctggaatagctactgcgttgatcctaagtaactatccagtgattctcaaggaggtaaatgagaaattcctggaggctggaattggcagagttaaagctaatctccagagccgtgtaaggaagggaagcatgtctcaggaaaagtttgagaaaaccatgtctctccttaagggttctcttgattatgaaagctttagagatgtggacatggtcattgaggctgttattgagaatatatctttgaagcaacaaatttttgctgatctggagaaatactgtcctcagcattgtatccttgctagcaacacatcgaccattgacttgaacaaaattggggagcggaccaagtctcaggatcgaattgttggagcacattttttcagtccagcacatatcatgccgctacttgaaatagttcggaccaatcatacctctgcccaagtaattgttgacctgttagatgttgggaagaagattaagaaaacaccagttgtggtgggaaactgcacagggtttgcagtgaataggatgttcttcccttacacacaggcagctatgttccttgttgagtgtggagcagatccatatctaatcgacagggcaatcagcaagtttggaatgccaatgggtcccttcagactgtgtgacctggttggatttggtgtggcgattgcaaccgcaacgcagtttatcgagaacttctcagaacggacttacaaatcaatgattattccacttatgcaagaggacaagagagctggtgaagccactcgcaaaggtttctatttgtatgatgataagcgcaaggcgaaacctgatcctgagctaaagaaatatatcgaaaaggcaagaagcatatctggagtaaagcttgaccctaagttggcgaatttgtcggagaaggacattattgaaatgacattcttcccagtagtaaacgaggcgtgtagggtttttgctgaaggtatcgctgtcaaagcagcagaccttgacattgctggcataatgggaatgggttttccaccttacagaggaggaatcatgttctgggctgattccatcggatcgaaatacatttactcgaggctggatgagtggtcgaaggcttatggtgaattcttcaagccttgtgcttttttggctgagaggggatctaaaggagttcttttgagcgctcccgtgaaacaagccagctcacggttgtaa

SEQ ID NO: 108 - DNA, 1314 bp, Arabidopsis thaliana
  misc_feature 1..1314: note = ORF of protein Mrb1
  source 1..1314: mol_type = unassigned DNA
atggcgacgaacggatcgtcggagcatcttctcccggagacgacaagaccaagcgcgatcatcaacaaacaacaaccggagtttcctgcatcacgattcacgttcatgtccctcgtgctctggttcgaccagtcaaatttcggtacggctctgctttcgtggtccgttttcttccttctcgtcgtcatcgttcctttgatctctcacttcttgctcgtttgctccgactgcgatttccatcaccgacgtccgtacgacgttattgtgcagctttcgttatcgattttcgcaggaatctcgttcgttagcttatcgatttggtcgagaaaatttgggatgcgacggtttttgttccttgataagctttgggatgttagcgacaaagttagaatcgaatacgaagctgaaattcagagatcactaaaacggctaatgatcttcgtcctcccatcactaacactcgaagcaacctacagaatctggtggtacatctctggcttcaaccagattccttacatcatcaatcctattcttagccacgtcgtcgcttgcactctccagctctcttcttggctttaccgtaactccttattcatcattgtctgcattctctacaaaatcacatgtcatctacaaactcttcgtcttgatgacttcgctcgttgcttcgcttctgagatcactgacgttagatcagctctcggcgagcatcaaaagattcgtcgtaatttgaggattgttagtcatcgattcaggagatttattcttttgtctttgattcttgttactgctactcagttcatggctttgcttactactacgagagctagtgttgctgttaatatctatgaagttggcgaactcgcgttatgttcgttgagtttggttacaggagtattcatatgtttgagaagtgcaacgaagataactcacaaagctcaatctgtaaccagccttgcagctaaatggaacgtttgcgcaacagttgactcgtttgatcatcttgatggcgaaactcctactggttccattattgaatctcaagtttccctacgcggcaatgcgatagagacgtctgatgatgaagaaggagaaggagacgatgatctcgataataccaagatacatcccatctatgccaatacaatttcttaccaaaaacgtcaagctctagtgacgtatctagagaacaacaaagctgggatcactgtgtatggattccttgtagatagatcatggttgaatacgattttcgggattgagctcgctcttctactatggttgctcaataaaacaatcgtgaatataccatga

SEQ ID NO: 109 - DNA, 1665 bp, Medicago truncatula
  misc_feature 1..1665: note = ORF of protein Nsp1
  source 1..1665: mol_type = unassigned DNA
atgactatggaaccaaatccaacatcagatcacattcttgattggcttgaaggttctgtctctttctttccatcatttttggatgatccatataacaatggttacatccatgagtatgagatatggaatcagaatcaagatataagtaaccagtaccaaattgatgccaatactaattcttctaatgccacaaatagcacaaccaacattgttgctgctagtactaccacaagtacaacatcccttgagcctaatagctttaataacatacccttttcagatttgccgaagaaacgaaatgccgaagatgaactaagtctcaaaaaacaacctcaaaaccaaaagaacaagagacttaagagtcgtccgatgaatgaaagtgataatggagatgcagctcttgaagggacagtggttagaaaatctggtgggaacaagaaaggtgcagctaaggccaatggaagtaacagtaacaatggaaacaataaggatggtaggtgggctgagcagttgctcaacccttgtgctgtagccataaccggtggaaatctgaatcgtgtgcaacatctcttatatgttctccatgagctagcctcaactaccggtgatgccaaccaccggcttgcagcacatggtcttcgagcattgacacaccatttatcttcatcttcgtcatctaccccatcagggactattacttttgcatctacagaaccgcgattcttccaaaagtcattactgaagttctatgagtttagcccttggttttcctttcctaataacatagcaaatgcttccattcttcaagttctagctgaagagccgaacaatttgcgcacccttcacatccttgacattggagtctctcatggtgtgcaatggccaacttttctagaggccttgagtcgtcgacctggtggacctcctcctcttgttcgccttactgtcgttaatgcctcttcatccactgaaaatgaccaaaacatggagaccccattttcaataggtccatgtggtgatactttctcttctggactccttggttatgctcagtccttaaatgtcaatctgcagataaaaaagcttgataatcatccattgcagacattgaatgctaaaagtgttgacacatcctctgatgaaacccttattgtctgtgctcagttcaggttgcatcacttgaatcataacaatcctgatgaaagaagtgagtttctgaaggtgttgagaggcatggagcctaaaggggtgatattaagtgagaacaacatggagtgttgctgcagtagttgtggcgatttcgccactggattctcacgaagagtggagtacttatggaggtttttagattcaaccagttcggcattcaaaaaccgtgacagtgatgaaaggaaaatgatggaaggcgaggctgcaaaagcattgacaaaccagcgtgaaatgaacgaaaggagagaaaaatggtgtgaaagaatgaaagaagcagggtttgcaggggaagtatttggagaggatgcaattgatggaggtcgagctttgttaaggaagtatgataataattgggagatgaaagtagaagaaaatagtacaagtgtggaactatggtggaagagccaacctgtttctttctgttctttgtggaaactggataaacaaccagagtag

SEQ ID NO: 110 - DNA, 1533 bp, Arabidopsis thaliana
  misc_feature 1..1533: note = ORF of protein Nsp1
  source 1..1533: mol_type = unassigned DNA
atgttgttggaagaaacagaaccaccaaaccagactctagatcatgtcctaagctggcttgaggattctgtgtccttatccccattaccaggatttgatgattcttatttgctccacgagtttgatgggtctcaaacgtgggaatgggatcagactcaagatccggagcatggttttattcaaagctatagtcaagatcttagtgcagcatatgttggttgtgaagcaactaacctggaagtggtaacagaagctccatccattgatttggatcttccacctgaaattcagcaaccaaacgatcagtccaggaaaaggagccacgacgggtttctcgaggcacaacaggtgaaaaaatcggcaaggagcaagagaaaagcaatcaagtctagtgagaagagctccaaagatggtaacaaggaagggagatgggcagagaagttgcttaacccttgtgccttggccattacggcaagtaactcatcaagggttcaacattacctttgtgttctctctgaactggcctcttcttctggtgatgctaatcgtcggcttgcagcttttggtcttcgggctttgcaacatcatctttcctcatcctctgtgtcatcatccttttggcctgtttttacttttgcttcagcggaagtgaagatgtttcaaaagactctgcttaagttctacgaggtaagcccttggtttgctttgcctaacaacatggcaaactcagctatcctgcagattttagcacaggatcccaaagataaaaaggatcttcatattattgatattggtgtttctcacggtatgcaatggcccactttgttggaggctctgagctgcagactagaaggacctcctcctcgtgttcgaataaccgttatatcagatctaaccgcagacatacctttctctgttggtccaccaggttacaattatggttctcaactcctaggctttgctcggtctctcaagatcaaccttcagattagtgtacttgacaagttacaactcattgatacctcacctcacgagaacttaatcgtgtgtgctcagtttaggctgcatcacctgaagcatagcatcaatgatgagagaggcgagactttgaaagcagtgagaagtttaaggccaaaaggagtggttctttgtgagaacaatggagaatgcagtagtagtgcggactttgcagcaggattctcgaagaaactggagtatgtatggaagtttctggattcaacaagctcgggatttaaagaagagaatagcgaagagagaaaactaatggaaggagaggcaacaaaggtgttgatgaatgcaggagatatgaatgaaggaaaagagaaatggtatgagaggatgagagaagctggtttttttgtagaagcatttgaagaagatgcagttgatggagccaaatccttactaagaaagtatgacaacaattgggaaataagaatggaagatggagatacctttgctggattaatgtggaaaggagaggcagtttccttttgttcattgtggaagtag

SEQ ID NO: 111 - DNA, 1581 bp, Brachypodium distachyon
  misc_feature 1..1581: note = ORF of protein Nsp1
  source 1..1581: mol_type = unassigned DNA
atgaaggccatggactaccatgagccggacgagcaagagctcaccaccgccgtgaccgcgatcgccgacggcggcggcctggactggctggaggactccatctccttgctgtccgcggacggggatctcagcggcagctacggttggtggtgcgatccaccgctgctgccggcgcagcaagacgacattgggatcgtcgtcgcgcagacgctgccgcctcccgcggcgctcacgacgaccggttcgccccccacaagcattgcgtcgccggccccggcgtcgtccaagaagcgcaagtcgtccccggcgtccggccatagcggcggcggcagccagcgtcggcgcgcggagcaggagagggagaggcctgcaggcggcggcggcaagaagaaggggggcggttcggactgcagggaggctcggtgggcggagcagctgctgagcccgtgcgcggtggccgtggaggcccggaacctgtcgcgcgtgcagcacctcttctacgtgctgggcgagctggcgtccttctcctcgggcgacgccaaccaccgcctggccgcctacggcctgcgcgcgctctccctccgcctccccgccgcggttggccccgccgccgcagccgccgtgcgcgtcccggccttgggcgccgagtgcccgtcgtcgttcgcggccgcggagccccgggtgttccgcgcgtcgctgatccggttccacgaggtgagcccctggttcgcgctccccaacgcgctggccaacgccgccatcgcccagcagctgctgcaggcgccaggccggccgctcagcctcgtcgacgtcggcgtctcgcacggcgtgcagtggcccaccctgctcgagtccctcacccgcctccccgccggccgcgcgccgccctccgtccgcctcacggtcgtctccgcggcagcagctacacgggcagcgccgttctcggcgtcgcctccggggtacgacttcgcgccgcacctcctccgctacgccaagtccatcaacctcgacctccacatcgcccgcgccggcgacttgtccgcccagctgggcgaggaggaggcgctcgtggtcgtgtgcctccagttccgcctcggccacgtggcccccgaggagcggacggaggtcctccggaaggtccgggatctgaaaccggatctcgtggttctctcggagctggacgttgtggtcggaggcggcagtgcaggcgcggcgggcgagttctcggcgaggctggagctgctgtggcggttcctggagtccacgtcggcagcgttcaaggggagggacggcgaggacaggcggctcatggaggccgaggcgggggcgcccgcgggcatgatcacggcgccggcgtccggggaagggagggaggcgtggcgggaccgcatgagagcggcggggttccaggaagcggccttcgggggcgaggccgtggagtcggccaggtcgctgctgaggaagtacgacagcggctgggagatggcgggagcagccagcggagccgccgcggtggccttgcgctggaagggccagccggtctccttctgctccttgtggcgcccggcgccgccgccgtag

SEQ ID NO: 112 - DNA, 1629 bp, Glycine max
  misc_feature 1..1629: note = ORF of protein Nsp1
  source 1..1629: mol_type = unassigned DNA
atgatcatggaaccaaacccaacaccagatcacagtctggactggtttgaaggttctgtgtcttactttccaccattcttagattatccatacaactctagtgacatccaagagtatcagttgtgggaacaagacatagctagcaactaccaaactgataccaataccagttcccctaatgccaccaacattgttaccatcacaacaccccttgagacttgtgactctaacaacctaccactttctgatttgcccaagaaaagaagtgccactgatgagtcaagtcccaaaccaccacaaaaccacaagcacaagaaaatcaagagcaagcccaggaacaatgaagcagataacggagatgcagaagcgacaacaacaacaacagcaacagttagaaaatctggtgggaacaagaaaggtggagctaaggccaatggaaataactgcaacaataaggatgggaggtgggcagaacagttgttgaacccttgtgctgcagccataactggtggaaatctgaaccgtgtgcaacacctcttgtatgttctccatgagttagcatcaccaactggtgatgctaaccacaggcttgcagcacatggtctcaaagcactgacacaacacttatcttcttctccaacttctacctcttcagggtctataacttttgcgtctgctgagccacggttcttccaaaagacactactcaagttctatgaagttagcccttggttttcctttcctaacaacatagcaaatgcttccattcttcaagttcttggggaagacactgataattcacgcacccttcacatccttgacattggagtctctcatggcatgcaatggccaacgtttcttgaggcgttgagtcgcagagctggtggacctcctccattggttcgcctcaccgtggtcactgcttcttcctccactgaaaatgacaccccattttgtattggtcctcctggtgataacttctcttctagactccttggttttgctcagtcaatgaatgttaatttgcagataaacaagctagataattgcccattgcattcactgaatgctcaaagtgttgacgcctccccggatgaaatctttgttgtgtgtgcacagtttaggttgcatcagttaaatcacaatgccccagatgaaaggagtaagtttctgacagtgttgaggaacatggagcccaaaggggtgatactaagtgacaacaacctggggtgttgttgcaattgttgtggggatttcgccactggattctctcggagggtggagtacttgtggaggtttttggactcaacgagctctgcattcaaaggcagggagagcgaggagaggagagtgatggaaggtgaggctgcgaaggcattgacaaaccagcgcgagacgaatgaagggaaggagaaatggtgtgagagaatgaaagaagcagggtttgtggaggaagtatttggagaagatgcaatagatggaggtagagctttgctgaggaagtatgaaagtaactgggagatgaaagtggaagatgacaatagaagtgttggactatggtggaagggacaaagtgtttctttctgttctttgtggaagctggatgggaatgatcagagctcaacatga

SEQ ID NO: 113 - DNA, 1632 bp, Glycine max
  misc_feature 1..1632: note = ORF of protein Nsp1
  source 1..1632: mol_type = unassigned DNA
atgatcatggaaccaaatccaacatcagatcacagtctagactggtttgaaggttctgtgtcttactttccaccactcttagattatccatacgactctggtgacatccaagactatcagttgtgggaacaagacataaccagccactaccaaactgataccaataccagttcttcccctaatgctaccaacatttctacaaccacaacaccccttgagacttgtgactctaataacaacctaccactttctgatttgccaaagaaaagaaatgccactgatgagtctagtcccaaaccaccacaaaaccacaagcacaagaaaatcaagagcaaaccaataaacaatgaagcctataccggacacgcagaagggacaacaacaacaacagttagaaaatctggtgggaacaagaaagggggagctaaggccaatggaaataactgcaacaacaaggatggtagatgggcagaacagttgctgaacccttgtgctgcagccataacaggtggaaacctgaaccgtgtgcaacacctctcgtatgttctccatgagttagcatcacctactggcgatgctaaccacaggcttgcagcacatggtcttaaagcactgacacaacatctatcatcttctccctcttcagggtctataacttttgcttccagcgagccgcggttcttccaaaagacactcctcaagttctatgaggttagcccttggttttccttccctaacaacatagcaaatgcttccatccttcaagttcttggcgaagacactgataataattcacgcacccttcacatccttgacattggagtctctcatggaatgcaatggccaacttttcttgaggccttgagtcgcagacctggtggacctcctccgttggttcgcctcaccgtggtcactgcttcttcctccactgaaaatgacacaccattttgtattggtcctcctggtgataacttctcttctagactccttggttttgctcagtcaatgaatgttaacttgcagataaacaaactagataactgtccattgcatactctgaatgctcaaagtgttgacacctccccggatgaaatctttgttgtgtgtgcacagtttaggttgcatcagttgaatcacaatgcccctgatgaaaggagtgagtttctgaaggtgttgagaaacatggagcccaaaggggtgatactaagtgacaacaacatgggatgttgttgcaattgttgtggggatttcaccactggattctctcggagggtggagtacttgtggaggtttttggactcaacaagctccgcgttcaagggccgggagagcgacgagaggagagtgatggaaggtgaggctgcgaaggcattgacaaaccagcgcgagacgaatgaagggaaggagaaatggtgtgagagaatgaaagaagcagggtttgtgggggaagtatttggagaggatgcaatagatggaggtagagctttgctgaggaagtatgatggtaattgggagatgaaagtggaagatgacaatacaagtgttggactatggtggaaggggcaaagtgtttctttctgttctttgtggaagctggatgggaattataagacctcaacatttacatcatga

SEQ ID NO: 114 - DNA, 1572 bp, Oryza sativa
  misc_feature 1..1572: note = ORF of protein Nsp1
  source 1..1572: mol_type = unassigned DNA
atgagctaccatgaggaggagcgccatggcggcaacggcttggactggttcgaggagtccatgtcctctttgctcgccgccgacgtcgacctcgctggtggtggtggtgatgcaggcggcggcggctacgcgtggtggtgggctgcttcgccggcggcgcagcaggatgacattgggagcgtcgtggcgcagacgctgtcgcctccgtcgacggcggcaccggccgcggcgtcgccgagcattgcgtcgcccgcggcgtcgtcgccgtcggacgtcccgtcgtcgtcgtccaagaagcgcaagtcccccgcgcacagggcgcccgggcacaccggcggcaagaaggggggcggcggcaagggcggcggcggagggtccgaccgggacatgaggtgggcggagcagctgctgaacccgtgcgcggtggccgtcgaggccggcaacctgtcgcgcgtgcagcacctgttctacgtcctcggcgagctcgagtccttctccggagacgccaaccaccgcctggccgcgcacgggctgcgcgcgctcgcgcgctggctccccgccgccgttggccccgcggctgcagcggccgtgcgggtgccgccctgctccgagcgccccacgacggcgttcgcggccgccgagccgcgcctgttccgcgcctccctcatcaggttccacgaggtgagcccctggttcgcgcttcccaacgcgctggccaacgccgccatcgcgcaggcctcgacgtgcggcgctgccggggcgacgccccggccgctccacgtcgtcgatctcggcgtctcccacggcgtgcagtggccgacgctcctcgagtcgctcacgcggcagccgggcggccgcgcgccgccgtccgtccgcctcaccgtggtcgggcccggggccacggccacctccccggtggcgccattctcggcgtcgcctcccgggtacgacttctcgccgcacctcctccggtacgccaagtccatcaacctggacctccgcatctcccgcgccgccaccctggacgacgccgtgcccggcgacgacggcgaggcgctcgtcgtgtgcctccagttcaggctcgggcacgccgccgccgaggagcgaagagaagtcctccgcaaggcgagaggcctcaatccggagctggtcgtcctctcggagctcgactccggcgtcggcgtcgtcggcggcgacggcggcagcgcggcgggcgagttcgcggcgaggctggagctgctgtggcggttcctggagtcgacgtcggcggcgttcaaggggaaggacgtggaggagcggcggctgctggaggccgaggccggcgcgatcctcgcggcggcggacgtcgccgccgctggggaagggagggaggggtggagggagcgcatggcggcggcggggttcgaggaggcgccgttcggcgcggaggcggtggagtccgccaggtcgctgctccgcaagtacgacagcgggtgggagatgtcagcgccgtcgccggcggccgccgcggtggcgctgcggtggaaggggcagccggtgtccttctgctcgctgtggcggccggcggcgtag

SEQ ID NO: 115 - DNA, 1629 bp, Solanum lycopersicum
  misc_feature 1..1629: note = ORF of protein Nsp1
  source 1..1629: mol_type = unassigned DNA
atggccattgaagaacctgagcctaattccaactccgaccccatatcagagtggctagctagtactttatccgatgttccatccttcttccatgaaccttacagttatgccgatgacttaaatttctacgcggacccatggtgggttcctgatcaagaggatcagatcgttaatcacaacatcatcatcgacaatacttgtaactcattcaacattagctccccagtcaacacagctattaacaatatccctttggagcccatcattttggatcatccacagccggtggatctgtctaagaaaaggaaaaaatctgatcaaaatccaaaggcgtcaaagaagaatcataaacttcagattaatgaggcagcacatgctcctactatagttgatcaagaaggagtgcaattaaaaaaatcaataggaccgaagagagtaacaacaggaaataacagtaacaacaaagaaggaagatgggcagaacatttgctcaacccctgtgctgctgcaatcactgtaggtaacatgaaccgcgtgcaacatctgttgtacgttctccatgaactggcgtcattcacaggcgatgccaaccacaggctagctgctcatggactccgcgccttgacacatcatctgtcttcccctggctcatcatcagcatcctctggaactattggagttactaatttctcttctgccaatcataaattcttcagggactcgttgattaatttcatcgacgttagtccctggttccgaatccccaataacattgcgaattcatctgttctccaaataattggacaacaggatcggctaaagaaccttcatatccttgatattggagtttctcatggttttcaatggccaactcttcttgaagaattgactaggcggtcaggtggaccgccgccactggttcgcctgaccgttattacaccaaccacagaaaacggagaattgacaggaactccatttgtgatcgggccacctggctatgatttttcctcgcaactactagcctatgcaaaggctatcaacatcaatctacagatcaacagactggataattttcctcttcaaaacctcaattcccaaatcataaattcttcatctgatgaaaccttagtcatctgtgctcaatttagactgcataacttgaatcatactatcccggatgatcgaacagatttattgaagatattgaagagtttggacccgaaaggattagttctcagcgagaataacacggagtgcagctgcaacagctgtggggactttgcgaccacgttctcaaggagagtggagtacttatggaggtttttggattccacgagtgttgcctacaaagggcgggagagcgaagaaaggaggatgatggaaggagaagcagcaaaggcattgacgaacatgggagaaatgaatgaaagaaaagagaaatggtgtgagagaatgaggagcgctggttttgtaaaagcggtgtttggtgaagatgccattgacggagctcgggctttgctgaggaagtatgacagcaattgggagataagggtggaggagaaagatggctgtgtggatctatggtggaaaggacagccaatttcattctgctcattatggaagatttag

SEQ ID NO: 116 - DNA, 804 bp, Solanum lycopersicum
  misc_feature 1..804: note = ORF of protein Nsp1
  source 1..804: mol_type = unassigned DNA
atgaccattgatgaaactacttctgatcccatctcagagtggctggatagtcccttatcatactttccgtcgttactcgatgatgagtcatatagcatcgatgatttactcgatgagtcatatagcctcgatgatatttacgaggactcatggtgggctcctaatgagagcataaaccaagaaatacatatcaataacaataacgataatagtttaccgtgcttcgatactagcttccctgtcacaaccgcgagcactgtctctctggagccggtcatttcaagtcatcttccgccattggattcatccaagaagaggaaagggaatgattctggttacaatcccaaggcgtcccggaagaatcagaaccgtaggatcaatgatgcagatacaagaagaacaacaggacacaagaaagcaacacacaagtctagtacaggaaataatggtaacaacagagaaggaagatgggcagagcagttacttaacccttgtgctgctgcaattacagcaggaaatctgaaccgtgtgcagcacttgttgtacgttctgcgcgagctctcctcactaaacggagatgccaaccatcggttggctgctcgtggactccaggcgctgacgtattatctttctactccatgcccaacttcactatcttctgcttctgctgctacttttgcttcaacaaatccgaagttcttcaagaactcactgatcaacttcaatgagatcagtccctggttccgcatacccaatagcatttttctcatggtgttcaatggccaacattgctcgaggagttga

SEQ ID NO: 117 - DNA, 1638 bp, Zea mays
  misc_feature 1..1638: note = ORF of protein Nsp1
  source 1..1638: mol_type = unassigned DNA
atgaacaatgagagagaaagccaagaaccggcagccgccacgacctcggattggctcgatgagtccatcgccttcctagccgccaacctcgacctcggctttaacgactatggctggccgtcggcatcggaagcgcaggagcggcacgacgacgtccgcagcatgatcgtggagacgctagcacctactgctacaaccctgcaccagccaagcctgaaccttgatattagcgttgcgtcgtcgtcgtcaccgatgacctcgcagcaggagtttgggcagcccaagaaacgcaaatcttctggaaacagcagcagccagcgaccccatgttggcggcggcggtcagcaagaaaagggcggagcgagctgcgccaggccgaagccgggcaagaagggcagtggcaagggcgccgcggcaactgtggacagggaggcgaggtgggcggagcagctgttgaacccgtgcgcggccgccatcgaagcgggcaacctgccgcgcacgcagcacctgctgtacgtgctcggcgagctggcctccttctccggcgaccccaaccaccgcctggccgcgcacgggctgcgcgccctcacgcgacgcctcccgcacgccacgggccaagcggctctggcgaccgtgaaggtgcccccttgcgagtgcccgacgtcggtgttctccggcgtcgacccgcggctgttccgagcctcgctcatcaggttcaacgaggtcagcccatggttcgccgtcccgaacgcgctcgccaactccgcgattgcgcaggcggcgtcgaggaagaagggcgcggtggccgagccccgtcaggttcacgtcgtggacatcggcgtctcgcacggcgtccagtggccgacgctgctcgaggcgctcacccgcgtgccgcggggctccacgccgccctccgtccgcctcacgctcgtcggtcccacggctacaccacccgtgcccttctccgcatcgcctccaggctacgactgctcgccgcagctgctccggtacgccaagtccatcgacctcgacctcgccatcgtccatgcgccatgtttggacaccttgcgcggcactctcacccccggcgagaccctcgtcgtctgcctccagttccggctcggcctcaccaccgccgacgatcaagcagctatattccgaaaggtcagggacctaaacccggagctaatggtcctcactgagctagacggcggtcgccgcaacgatgacagcgcaaccagcgagttcgcggcgaggctggagctgctttggagattccttgactcgacggcggcggcgttcaaggggatggacgcgtatgagaggcgggtgatggaagcggaggccgggacagcattggcgctcacgccgcggtgcggagcgaccgtggacggggggagggaggcgtggcgagcgcgtttggccgcggcggggttcgaggaagcggcgttcgggggcgaggcggtggagacagcgaaggcgctgctgaggaagtacgatggcgggtgggagctggtgccaccgtctccgtccgcggcggcagcggtggggctccggtgggagggtcagcccgtgtcattctcatccatgtggcggccggcgcaggccagcccggtgctgattgggccttgcacataa

SEQ ID NO: 118 - DNA, 885 bp, Zea mays
  misc_feature 1..885: note = ORF of protein Nsp1
  source 1..885: mol_type = unassigned DNA
atggcagccacaatccggcgctcgtgcgcatgtatcgcgacaaaacggaaatatgcaactaactacatgcgaggcttatccaacatcttcttgcaacgagcaggagcagcacgggtgtcttcttgcaagattgcatgcatgaagcccatgagctgccacgagcaggagcagcacgcggagctcgccgccgccacgaccaacagcggcttcgactggcttgaggactccgtctccttcctcacagcagacgttgacgccgccggctgccgcggctacgggtggtgtccgcccctagcgcacacatcgccaagcattgcgtcaccggccgtgtcggagcctttgtccaagaagcgcaagtccccagcgcacagagcgtccgcccacagtggcagcaaccagcgtcgtcgcgctgaccaggcggacaggccaggtgctggcggcggcaagaagggcgggggagcgggatccgaccgcgacacgaggtgggcggagcagctcctgaacccgtgcacgaccgccgtcgaggccggcaacctgacgcgcgtgcagcacctgttctacgtgctcggcgagctcacctccttctccggcgacgccaaccaccgcctggccgcgcatgggctgcgcgcgctcgcccgcagcaggctccccgccgccgtcgggccggcggccgcgacggccgtgcgggtgccgccctgcgactgccccacgccggcgttcgcgggcacggagccgcggctgttccgcgcgtcgctcatcaggttccacgaggtgagcccctggttcgcgctggccaacgccgccatggagtggaatagggagaagggtaacagatgtagagacgccgtgagtgtcactggtttaatcgtagatgttaccttgtaa

SEQ ID NO: 119 - DNA, 1581 bp, Zea mays
  misc_feature 1..1581: note = ORF of protein Nsp1
  source 1..1581: mol_type = unassigned DNA
atgaagcccatgagctgccacgagcaggagcacccggagctcgccgccaccacgaccaacagcggcttcgactggcttgaggatcccatctccttcctcaccgcagacgtcgacatcgccggcggcggctatgggtggtggtccgatcccgcggcggcgcagcaagatggtattggcagcgtcgtggcgcagaccctttcgcccccggcgccgctgctggcgcacacctcgccgagcattgcgtcaccggtcgtgtcggagccttcgtccaagaaacgcaagtccccggcgcacagggcgtccgccagtactggcagcaaccagcgtcgccgcgccgaccaagacaggccaggtgctggtagcagcagcggcggcagcaagaggagcggctccaagggcggtggagcgggatccgaccgggacacgaggtgggcggagcagctcctgaacccgtgcgcgaccgccgtcgaggccggcaacctgtcgcgcgtgcagcacctgttctacgtgctcggcgagctcgcctccttctccggcgacgccaaccaccggctggccgcgcacgggctgcacgcgctggcccgcatgctccccgccgccgtcggcccggcggccgcggcgaccgtgcgggtgcctccctgcgagtgcccgacgccggggttcgcgggcgcggagccgcggctgttccgcgcgtcgctcatcaagttccacgaggtgagcccgtggttcgcgctccccaacgcgctcgccaacgccgccatcgcgcaggccgccgcgtcgggctcgggcgggcgcgggggctccgagccccggtcgctccacgtcgtcgacctcggcgtctcgcacggcgtgcagtggccgacgctgctggagtcgctgacccggctgcccggcgggcgcgcgccgccctccgtccgcctcaccgtcgcgggcgccccgccggcgcccttctcggcgtcgcctccggggtacgacttctcgccgcacctcctccggtacgccaagtccatcgacctgcggctcgcgatcagccgcgcggcctccctagactccgtgcacgggctcgcctcctccccgtcgggcgaggcgctcgtcgtgtgtctccagttccggctcggccacgccggcgccgacgagcggacagagattctaaggaaggtccggggtctcaacccggagctggtggtggtctcggagctcgacggcgggggcgacggcaccgcggcgggcgagttcacggcgaggctggagctgctgtggcggttcctggagtcgacgtccgcggcgttcaaggggagggatggggaggagaggcgcctgctggaggccgaggctggcacgtccgtcgcgtcggaggcggcaggggcagggagggaggcgtggcgggagcgcatggtggtggccgggttcgaggcggcggcgttcggagacgaggcggtggagtcggccaagtcgctgctgaggaagtacgacggcggctgggagatgtcggcgtcggggacggccggcgcggtggcgctgcggtggaaggggcagccggtgtcgttctgctcgctgtggcggccggcgtgcagcggctaa

SEQ ID NO: 120 - DNA, 1608 bp, Zea mays
  misc_feature 1..1608: note = ORF of protein Nsp1
  source 1..1608: mol_type = unassigned DNA
atgaagcccatgagctgccacgagcaggagcagcacgcggagctcgccgccgccacgaccaacagcggcttcgactggcttgatgaccccatctccttcctcacagcagacgttgacgccgccggctgcggcggctacgggtggtggtcagacgatcccgcggcggcgcagcaagataatattggcagcgtcgtggcgcagacgctttcgcccccggcggcgccgctgctgacgaccagtccgcccctggcgcacacctcgccaagcattgcgtcaccggccgtgtcggagccttcgtccaagaagcgcaagtccccggcgcacagggcgtccgcccacagtggcagcaaccagcgtcgccgcgctgaccaggcggacaggccaggtgctggcggcggcaagaagggcgggggagcgggatccgacgaccgcgacacgaggtgggcggagcagctcctgaacccgtgcgcggccgccgtcgaggccggcaacctgacgcgcgtgcagcacctgttctacgtgctcggcgagctcgcctccttctccggcgacgccaaccaccgcctggccgcgcacgggctgcgcgcgctcgcccgcagcaggctccccgccgccgtcgggccggcggccgcggcggccgtgcgggtgccgccctgcgactgccccacgccggcgttcgcgggcgcggagccgcggctgttccgcgcgtcgctcatcaggttccacgaggtgagcccctggttcgcgctccccaacgcgctggccaacgccgccatcgcgcgggccgcgtcgcgcggcgggtccacgcccgcggagcagccccggccgctccacgtcgtcgaccttggcgtctcgcacggcgtgcagtggccgacgctgctggagtcgctgacccgcctgtccggcggccgcgcgccgccctccgtctgtctcatcaccgtcgcggccgccccgccggcgcccttctcggcgtcgcctccggggtacgacttctcgccgcacctcctccggtacgccaagtccatcagcctgcagctcgccatcagccgcgcggcctccctggactccgtgcgtgggttggcctccccgtcggcgtcgggcgaggaggccctcgtcgtgtgtatccagttccggctcggccacgccagcgccgacgagcggacagagatcctaaggaaggtccgaggtctcgacccggagttggtggtggtctcggagctcgacggcgggggcgacggcaccgcggcgggcgagttcacggcgaggctggagctgctgtggcggttcctggagtcgacgtccgcggcgttcaaggggagggacggggacgagaggcgtctgctggaggccgaggctggcgcgtctctcgtcgcgtcggaggcgccggcgctggaagggagggaggcgtggcgagaggccatggcggcggccgggttcgaggaggcgtcgttcggagacgaggcggtggagtcggccaagtcgctcctgaggaagtacgacggcggctgggagatgtcggcgtcgggggcggccggcgcggtggcgctgcggtggaaggggcagccagtgtccttctgctcgctgtggcggccggcgtgcagcggctaa

SEQ ID NO: 121 - DNA, 1530 bp, Brassica rapa
  misc_feature 1..1530: note = ORF of protein Nsp1
  source 1..1530: mol_type = unassigned DNA
atgtcgctagaagaaacagaaccaccaagccaaactctagataatgtcctaggctggtttgatgattctatctccttaacgccattacctggattcactgattctgatttgctcaacgagtttgactggtctcaaacgtgggaatgggatcatcagactcaagctcaagatccaggatgtgattttcttcatagctatagtcaagatcttgatgcatatatagggtgtgaagcaactaatcttgaagtggtaacaggagttccagtcattgattcagatcctccaccagtggttcagctaccaaacgatcagaccaagaaacggaaccgtgatgagcttaacgatccccacgttgtgaaaagatcagcaaggagcaagaaaaaagcagacaagtctactgaggtgagccgcaaagatagcaacaaggcagagagatgggcagaacaacttcttaacccttgtgccttggcaatcacggcaaggaactcatctagggttcaacattacatttgtgttctctctgaacttgcctcttcttgtggtgattctaatcataggcttgcatcttttggtctttgtgctttgaaaaaacacatttcaacttcctttgtgccatcatccactcttacttttgcttcagcagaagtgaagatgtttcagaagactttgctcaagttctatgaagtaagcccttggtttgctttgcccaacaacatggcaaactcagccatcctacagattctatcacaagagacaagagataaaacagatcttcacattcttgatattggtgtttctcatggtatgcaatggcccactttgctggaagctctctgctgcagaccagaaggacctcctcttcaagttagaatcaccgttgtatccgatctaaccgcagacatacctttctctgttggtccaccatcttacaactatgcctctcagctcattggctttgctaggtccctcaacatcaacctccagataagtgtaattgacaagtctcaactcattgatacctcacctcatgagaccttcatcgtctgtgctcagtttaggctgtaccaactgaagaatagcatcactgatgagagaagcgaggctttgaaagcacttagaagtttgaatcctaaaggagttgttctttgtgaatacaatggagaaggcagtagcagtgtggactttgcagcagacttctcaaggaaactggactatctatggaagtttttggactcaacaagctatggattcaaagaagagaacagcgaagagaggaatctaatggaaggagaggcaacaaaggtgttgatgagctcaggtgatactaatgaagggaaagagatatggtatgagaagatgagaaaagctggcttctcagcagaagcatttggggaagatgcaatagatggagccaaatctttactcagaaagtatgataacaattgggaaataaagatggaagatgaagatacctttgctggtttactatggaaaggagaagaagtttccttttgttccttgtggaagtag

SEQ ID NO: 122 - DNA, 1281 bp, Hordeum vulgare
  misc_feature 1..1281: note = ORF of protein Nsp1
  source 1..1281: mol_type = unassigned DNA
cttcgtcgtcgcctccgcgtccaactccccctccccgtccgcctggcgctgtctggctccggtcatggtagcagcaaccagcgccgccgaatcgagcaggacaggccgggaggcggagcggggtcggaccgggacgcgaggtgggcggagcagctgccgggtccctgcgccgtcgccgtcgaggcgcgcaacctgtcccgcgtgcagcacctcttctacgtgctcggcgagctcgcggccttctccggtgacgccaaccacaggcttgccgcgtacgggctgcgggcgctctcgcttcgcctccctgccgcggttggccccgcggccgccgcggccgtgagggtgccggccgcatgcgagtgcctgacgccgtcgttcgccggcgcggagccacggctgttccgcgcgtccctcatcaagttccacgaggtcagctcgtggttcatgctccccaacacgttggcgaacgccgccatctcgcagctggcctccccggcgctccacctcatcgacattggcgtctcgcacggggtgcggtggccgaccttgctggagtccctcacccgcatggccgccggacgggcgccgccgtccgtccggctgacggtcgtggccaccccgccggtgcccttctcggcctcacctccggggtacgacttctctccgcacctcctccggtacgccaagtccattaagctggacctcgagatcggccgcgccgcctcactagactccgcgcacggcttctgcaccgctcccgacgaggcgctcgtggtgtgcctccagttcctgctgggccatgtcaccgccgaggagaggacggacatcatccgcagggtcaaagacctcaagccgcagctggtggtgctatcagagctcgacgtcgggggcaacggcagcgcggcgggcgagttcacggcgaggctggagctgctgtggcagttcttggagtcgacgcacgcgacgttcaaggggagggacgggaaggacaggcggttcctggaggccgaggcagggaccgccgtcgcggtggccgcggccagggaggccgtggggcgagaggggtggcgggaccgcatgacggcgacggggttcgagcaggtggcgttcgggggcgaggcggtggagtcggccaggtcgctgctgaggaagtacgacaacgggtgggagagtccacggcggcggcggcgacgggaggcggcggcgccgtggcgctgcggtggaaagggcagccggtgtccttctgctcgctgtggccgccggcgtagcagcggtagctga

SEQ ID NO: 123 - DNA, 1446 bp, Hordeum vulgare
  misc_feature 1..1446: note = ORF of protein Nsp1
  misc_feature 1..26: note = n is a, c, g, or t
  misc_feature 1183..1187: note = n is a, c, g, or t
  source 1..1446: mol_type = unassigned DNA
nnnnnnnnnnnnnnnnnnnnnnnnnncaccgcagtagagccccaccgcccccacaccatagcagagccccaccgccccgcagcccagcccaaccccatcgcgccgccgctcgcgttggcccacgcccacccctcgccaagcattgcatcgcccgcggcgtccgagccgtcgcccgggaagcgcaagtctccggtgcgtgtggggtctggctccggtcagggtagcagcaaccagcgccgccgaactgagcaggacaggccgggaggcggcaagaagggcggcagagcggggtcggaccgggacgcgaggtgggcggagcagctgctgggtccctgcgccgtcgccttcgaggcgcgcaacctgtcccgcgtgcagcacctcttctacgtgctcggtgagctcgcggccttctccagacaccccaagcacaggcttgccgcgtacgggctgcgggcgctatcgcttcgcctccctaccgcggttggccccgcggccgccgcagctgtgagggtgccggccgcatgcgagtgcccgacgccgtcgatcgccggcgcggagccacgactgttccgcgcgtccctcgtcaagttccacgaggtcagcccgtggttcacgctcctcaacacgctggcgaacgccgccatctcacagctggcctctgcggcgccccgtccgctccacctcgtcgacattggcgtctcgcacggggtgcagttgccgaccttgctggagtccctcacatgcatggccgccggacgggcgccgccgtccgtccggctgacggtcgtggccaccccgccggtgcccttctcggcctcgcctccggggtacgacttttcttcgcacctcctccgatacgccaagtccattaagctggacctcgagatcggccgggccacatcactagactccgcgcacggcttctacaccgctcccaacaaggcgcgcgtggtgtgcctccagttccggctgggccatgtcaccgccgaggagaggatggacatcatccgcagggtgaaagacctcaagccgcagcgggtggtgctatcagggctcgacgtcggtggcaacggcagcgtggcgggcgagtccacggcgaggctggagctgctgtggcggttcttggagtcgacgcacgaggcgttcaaggggagagacgggaaggacaggcggctcctggagnnnnnggcggggaccgccgtcgcggtggccgcggccagggaggccacggggagaaaggggtggcgagaccgcatgacggcggcggggttcgaccaggtggtgttcgggggcgaggtggtggagtcggccaggtcgctgctgaggaagtacgacaacgggtgggagatgtccacggtggcggcggcgaagggaggcggcggcgccgtggcgctgcggtggaaagggcagccggtgtccttctgctcgctgtggcggccggcgtag

SEQ ID NO: 124 - DNA, 429 bp, Hordeum vulgare
  misc_feature 1..429: note = ORF of protein Nsp1
  misc_feature 1: note = n is a, c, g, or t
  source 1..429: mol_type = unassigned DNA
naagacctgaagccgcagctggtggtcctatcagagctcgacgtcgggggcaacggcagcgcggcgggcgagttcacagcgaggctggagctgctgtggcggttcttggagtcgacgcacgcggcgttcaaggggagggacggggaggacaggcggctcctggaggcggaggcgggaaccggcgtcgcggtggccgcggccagggaggccatggggcgagaggggtggcgagaccgcatgacggcggcggggttcgaccaggtggcgttcgggggcgaggcggtggagtcggccaggtcgctgctgaggaagtacgacaacgggtgggagatgtccacggcggcggcggcgacgggaggcggcggcgccgtggcgctgcggtggaaagggcagccggtgtccttctgctcactgtggcggccggcgtag

SEQ ID NO: 125 - DNA, 1356 bp, Hordeum vulgare
  misc_feature 1..1356: note = ORF of protein Nsp1
  source 1..1356: mol_type = unassigned DNA
acaaactgcagatacgtatataccagtcccctctttctccgtcggggtcggacgtgcctaccggagtgtcggaggtgcaaccgcgcatgcgcatgcgcagcaatgcaagcaggcggccgaggcaaggcgacgacgaacaagcagggatggatcgatgccttctcctcttccgaaatgggcccttgggcgtcgcggagcggggtcggaccgggacgtgaggtgggcggagcagctgctgggtccctgcgtcgtcgccgtcgaggcgcgcaacctgtcccgcgtgcaacacctcttctacgtgctcggcgagctcgcggccttctccggcgacgccaaccacaggcttgccgcgtacgggctgcgagcgctctcgcttcgcctccctgccgcagttggccccgcggccgccgcggccgtgagggtgccggccgcatgcgagtgcccgacgccgtcgctcgccgggcggagccacggatgttccgcgcgtccctcatcaagttccacgaggtcagcccgtggttcacgctccccaacacgctggcgaatgccgccatctcgcagctggcctctgcggcgccccggccgctccacctcgtcgacattgacgtctcgcacgaggtgcagtggccgaccttgctggagtccctcacccgcatggccgccggacgggcgccgccgttcgtccggctgacggtcgtggccaccccgccggtgcccttctcggcctcgcctccggggtacgacttctctccgcacctcctccggtacgccaagtccattaagctggacttcgagatcggtcgcgccgcctcactagactccgcgcacggcttctgcaccgctcccgacgaggcgctcgtggtgtgcctccagttccggctgggccatgtcaccgccgaggagaggacgtacatcatccgcagggtcaaagacctcaagccgcagctgatggtgctatcagagctcgacgtctggggcaacggcagcacggcgggcgagttcacggcgaggctggagctgctgtggcggttcttggagtcgacgcatgcggcgttcaaggggagggacggggaggacaggcggctcctggaggccgaggcggggagcgccgtcgcggtggccgcggccagggaggccgtggggcgagaggggtggcgggaccgcatgacggcggcggggttcgaccaggtggcgttcgggggcgaggcggtggagtcggccaggtcgctgctgaggaagtacgacaacgggtgggagatgtccacgacggcggcggcgacgggaggtggcggcgccgtggcgctgcggtggaaagggcagccggtgtccttctgctcgttgtggtggcgggcgtag

SEQ ID NO: 126 - DNA, 1134 bp, Hordeum vulgare
  misc_feature 1..1134: note = ORF of protein Nsp1
  source 1..1134: mol_type = unassigned DNA
gcaccaccaccgtgtgcatttaccaaattaactaagcgccctagcctgcaccttcgccgtcgcctccgcgtccaactccccctccccgtccgcctggcggtgtctggctccggtcatggtagccgcaaccagcgccgccgaaccgagcaggacaggccgggaggcggcaagaagggcggcggagcggggtcggaccgggacgcgaggtgggcggagcagctgctgggtccctgcgccgttgccgtcgaggcccgcaacttgtcccacgtgcagcacctcttctacgtgctcggtgagctcgcggccttctccggcgacgccaaccacaggcttgccgcgtacgggctgcgggcgctctcgcttcgcctccctgccgcggttggccccgctgccgccgcggccgtgagggtgccggccgcatgcgagtgcccgacgccgtcattcgccggcacggagccacggctgttccacgcgtccctcatcaaattccacgaggtcagcccgtggttcacgctccccaacacgctggcgaatgccgccatctcgcagctggcctctgcggcgccccggccgctccacctcatcgacattggcgtctcacacggggtgcagtggccgaccttgctcgagtccctcacccgcatggccgccggacgggcaccgctgtccgtccggctgacggtcgtggccaccccgccggtgcccttctcggcctcgcctccggggtacgacttctctccgcacctcctcccgtacgccaagtccattaagctggacctcgagatcggccgcgccgcctcactagactctgcgcacggcttctgcaccgctcccgacgaggcgctcgtggtgtgcctccagttccggctgggccatgtcaccgccgaggagaggacggacatcatccgcagggtcaaagacctcaagccgcagctggtggtgctatcagagctcgacgtcgggggcaacgacagcgcggcgggcgagttcactgcgaggctggagctgctatggcggttcttggagtcgacgcacgcggcgttcaaggggagggacggggaggacaggcggctcttggaggccgaggcggggaccgccgtcgcggtggccgcggccagggaggccgtggggtga

SEQ ID NO: 127 - DNA, 789 bp, Hordeum vulgare
  misc_feature 1..789: note = ORF of protein Nsp1
  source 1..789: mol_type = unassigned DNA
agcggcaaccagcgccgccgaaccgagcaggacaggccgggcggcggcaagaagggcggcggagcggggtcggaccgggacgcgaggtgggcggagcagctgctgggtccctgcgccgtcgccgtcgaggcgcgcaacctgtcgcgcgtgcagcacctcttctacgtgctcggcgagctcgcggccttctccggcgacgccaaccacaggctggccgcgtacgggctgcgtgcgctctcgcttcgcctccctgctgcggttggccccgcggcagccgcggccgtgagggtgccggccgcatgcgagtgcccgacgccgtcgttcgccggcgcggagccacggctgttccgcgcgtccctcatcaagttccacgaggtcagcccgtggttcacgctccccaacacgctggcgaacgccgccatctcgcagctggcctctgcggcgccccggccgctccacctcgtcgacattggcgtctcgcacggggtgcagtggccgaccttgctggagtccctcacccgcatggccgccggacgggcgccgccgtccgtccggctgacggtcgtggccaccccgccggtgcccttctcggcctcgcctccggggtacgacttctccccgcacctcctccggtacgccaagtccattaagctggacctcgagatcggccgcgccgcctcactagactccgcgcatggcttctgcaccgctcccgacgaggcgctcgtggtgtgcctccagttccggctgggccatgtcaccgccgaggagaggacggacatcatccgc

SEQ ID NO: 128 - DNA, 693 bp, Hordeum vulgare
  misc_feature 1..693: note = ORF of protein Nsp1
  source 1..693: mol_type = unassigned DNA
atgctccacctcctcctacaacgaaaaactgttacacctccacgaaaaaaagagccaatcaagtacagaaatttacgaacaatgcaccttcgccgtggcctccgcgtccaactccccctccccgtccgcctggcggtgtctggctccggtcatggtagcagcaaccagcgccaccgaaccgagcaggacaggccgggaggcggcaagaagggcggcggagcggggtcggaccgggacgcgaggtgggcggagcagctgctgggtccctgcgctgtcgccgtcgaggcgcgcaacctgtcccgcgtgcagcacctcttctacgtgggcgagctcgcggccttctccggcgacgccaaccacaggcttgccgcgtacgggctgcgggcgctctcgcttcgcctccctgccgcggttggccccgcggccgtgagggtgccggccgcatgcgagtgcccgacgccgtcgttcgccgccgcggagccacggctgttccgcgcatccctcatcaagttccacgaggtctgctcgtggttcacgctccccaacacgttggcgaacgccgccatctcgcagctagcctccccggcgctccacctcatcgacattggcgtatcgcacggggtgcagtggccgaccttgctggagccctcacccgcatggccgccggacgggcgccgccgtccgtccggctga

SEQ ID NO: 129 - DNA, 1629 bp, Solanum tuberosum
  misc_feature 1..1629: note = ORF of protein Nsp1
  source 1..1629: mol_type = unassigned DNA
atgaccattgaagaacctgagcctaattccaactccgaccccatatcagagtggttagctagtactttatccgatgttccatccttcctcgatgaaccttacagttacaccgatgatttaaatttctacgcggactcatggtgggttcccgatcaagatgatcagatcgttaatcacaacatcatcatcgacaatacttgtaactcattcaacattagctccccagtcaacacagctattagcaatatccctttggagcccatcattttggatcagccacagccggtggatctgtctaagaaaaggaaaatatctgatcaaaatcctaagacgtcaaagaaaaatcataaccgtcagattaatgaggcagaaaatgctcatactataggtgatcaaggaggagtgcaagtaagaagaccaaagagagtaacaacaaaatcaaccggaaataacagtaacaacaaagaaggaagatgggcagaacatttgctcaacccttgtgctgctgcaatcactgtcggtaacatgaaccgcgtgcaacatctgctgtacgttctccatgagttggcgtcgttcacaggtgacgccaaccacaggctagctgctcatggactccgcgccttgacacatcatttgtcttcccctggctcatcatcagcatcctctggaactattggagttactaatttctcttctgcgaatcataaattcttcagggactcgttgatcaatttcatcgacattagtccctggttccgaatccccaataacattgcgaattcatctgttctccaaataattggagaacaggatcggctaaagaaccttcatatccttgatattggagtttctcatggttttcaatggccaactcttcttgaagaattgactaggcggtcaggtggaccgccaccactggttcgcctgaccgttattacacccacaacagaaaacggagaattgagaggcactccatttgtgatcgggccacctggttacgatttttcctcgcaactgctagcctatgctaaggctatcaacattaatctacagatcaacagactggacaattttcctcttcaaaatctcaattcccaaatcataaattctacatctgatgaaaccttagtcatctgtgctcaatttagactgcataacttgaatcatactatcccggatgatcgaacagatttgttgaagatattgaagagtttggacccgaaaggattagttctcagcgagaacaacacagagtgtagctgcaacagctgtggggactttgcgacaatgttctcaaggcgagtggagtacttatggaggtttttggattccacaagtgtggcctacaaagggcgggagagcgaagaaaggaagatgatggaaggagaagcagcaaaggcgttgacgaacatgggagaaatgaatgaaagaaaggagaaatggtgtgaaagaatgaggaacgttggctttgtaaaagaggtgtttggtgaagatgccattgacggagctcgggctttgctgaggaagtatgacagcaattgggagatgagggtggaggagaaagatggctgtgtggatctatggtggaaagggcagccaatttcattctgctcattatggaagatttag

SEQ ID NO: 130 - DNA, 1662 bp, Solanum tuberosum
  misc_feature 1..1662: note = ORF of protein Nsp1
  source 1..1662: mol_type = unassigned DNA
atgaacattgatgaacctacttctgatcccatctcagagtggctggatagtcccttatcatattttccgtcgttactcgatgagtcatatagcatcgatgatttattcgatgagtcatatagcctcgatgatttattcgatgagtcatatagcctcgatgatatttacgaggactcgtggtgggctcctagtgagagtataaaccaagaaatacatatcaataacaataacaataacagtttaacgtgcttcgacactagcttccctgtcacagccgcgagcactgtctctttggaaccggtcatttcaagtcacccttcgccattggattcatccaagaaaaggaaagggaatgattccggttacaatcccaaggcgtcccggaagaatcagaaccgtcggatcaatgatgcagataaaagaagatcaacaggacacaagaaagcaacacacaagtccacaggaaataatggtaacaacagagaaggaagatgggcagagcagttacttaacccttgtgctgctgcaattactgcaggaaatctgaaccgtgtgcagcacttgttgtacgttctgcgcgagctctcctcactaaacggagatgccaaccatcggttggctgctcgtggactccaggcgctaaagtattatctttctactccatgcccaacttcactatcttctgtttctgctggtggtgttgctacttttgcttcaacaaatccaaagttcttcaagaactcactgatcaacttcaatgagatcagtccctggttccgcatacccaatagcattgcaaactcctcaatactccaaattcttgggaaacacgattcatcccagaacctacacattcttgatattggagtttctcatggtgtccaatggccaacgttgcttgaggagttgacccatagagcaggaggtccgccaccattggttcgtctaacagtaataacacctaccatagaagatgatcaacaaagaaatagtccatttgtagttggtccaggtggttacaacttttcggtgcaactccttgcctttgccaaggctattaacatcaatctacaaatcaacagactggaaaattacccacttcagaatcttgattcccaagtgataaattcatccccagatgaaatcttggttatttgtgcacagttcagactccataacttgaaacacaacaatccagacgagaggacagagttgttgaaaagactaaagagtttggagccaaaaggactggttttcagtgagaacaacatggattgcagctgcaacagttgcggggacttcacaacaagattctcaaggcgagtggagtacttgtggaagttcttggactccaccagtgtagcatacaaaggacgtgagagcgatggaaggaggatgatggaaggagaagcagcaaaggctttgacaaacatgggagagatgaatgagacaaaggagaaatggtgtgaaagaatgaggactgttggtttcgtatggaagatgtttggagatgatgccattgatagagctcgtgcattgttgaagaagtatgacaacaactgggagatgagagttgatgagaaggatggctgtgttggactgtggtggaaaggagatcctgtttcattttgttcattatggaagatagatcccaacacatga

SEQ ID NO: 131 - DNA, 1251 bp, Triticum aestivum
  misc_feature 1..1251: note = ORF of protein Nsp1
  source 1..1251: mol_type = unassigned DNA
atggtgtccggctccggccatagcagcagcaacaaccagcgtcgccgaactgagcaggataggccgggcggcggcaagaagggcggcggcgcggggtcggacagggacgcgaggtgggcggagcagctgctgggcccgtgcgccgtcgccgtcgaggcgcgcaacctgtcgcgcgtgcagcacctcttctacgtgctcggagaactcgcggccttctccggcgacgccaaccacaggctagccgcgtacgggctgcgggcgctctcgcttcgcctcccagccgcggtgggccccgcggccgccgcggccgtgagggtgccggccgcctgcgagtccccgacgccgtcgttcgctggcgccgagccacggctgttccgcgcgtccctcatcaagttccacgaggtcagcccgtggttcacgctccccaacacgctggcgaacgccgccatctcgcagctggcttcggcagcgccccggccgctacacctcgtcgacattggcatctcacacggggtgcagtggccgactttgctggaatctctcacccgcatggccgctgggcgagcaccgccatccgtccggctgactgttgtggccaccccgccggtgcccttctcggcctcgcctccggggtacgacttctcgccgcatctcctccggtacgccaagtccattaagctggacctcgagatcagccgcgccgcttcactagaccccgcgcatggcttgtgcaccactcccgacgaggcgctcgtggtgtgcctccagttccggctgggccatgtcaccgccgaggagaggacggacatcatccgcagggtcaaaggcctcaagccgcacctggtggtcctatcggagctcgacgtcgggggcaacggcagcgcggcaggcgagttcacggcgaggctggagctgctgtggcggttcttggagtccacgcacgcggcgttcaaggggagggacggggaggacaggcggctcctggaggccgaggcgggcaccgccgtggcggtggccgcggccagggaggccatggggcgggagggctggcgggaccgcatgacggcggcggggttcgagcaggtggcgttcgggggcgaggcggtggagtcggccaggtcgctgctgaggaagtacgacaacgggtgggagatgtccacggcggcggcggccacgggaggcggcggcgccgtggcgctgcggtggaaagggcagccggtgtccttctgctcgctgtggcggccggcgtag

SEQ ID NO: 132 - DNA, 213 bp, Triticum aestivum
  misc_feature 1..213: note = ORF of protein Nsp1
  source 1..213: mol_type = unassigned DNA
ccgccttccgtccggctgacggtcgtggccaccccgccggtgcccttctcggcctcgcctcccggatatgacttcgcgccgcatctcctccggtacgccaagtccattaagctggacctcgagatcggccgcgccgcttcactagaccccgcgcatggcttgtgcaccactcccgacgaggcgctcgtggtgtgcctccagttccggctcggc

SEQ ID NO: 133 - DNA, 1605 bp, Lolium perenne
  misc_feature 1..1605: note = ORF of protein Nsp1
  source 1..1605: mol_type = unassigned DNA
atgaaggccatgggctgccatggacaagaggaccaaaagctcgccctcgtcgcagccagcgacgatggcttcgactggctggaggactccatctccttgctcgctgcggacatcgatctcggtggcagctacgggtggtggtgtgatccattgccggcgcagcagcaagatgacattgggagcgtcgtggcgcagacgctgtcgcctccagcgccacagacgacaagctcgcccctggcccacgcctcaccgagcattgcatcgccagcggtgtcatccccgtccacggagccgtcgtccaagaagcgcaagtctccgacgcacagggtgtccggccatagcagcagcaaccagcgtcgccgcgccgaacaggagaggccaagtggcggcggcagcaagaagggcagcagcaagggcggcggagctggttcggacagggacgcgaggtgggcggagcagctgctgagtccgtgcgccgttgccgtcgaggcacgcaacctgtcgcgcgtgcagcacctgttctacgtgctcggcgagctcgcgtccttcgccggcgatgccaatcaccggctggccgcgtatgggctgcgagcgctctcgcttcgcctacctgcagcagtgggcacagctgccgcagctgcggtgaaagtgccggactgcgagtgcccgacgccgtcgttcgctggcgcggagccccggctgttccgcgcgtccctcatcaagttcaacgaggttagcccgtggttcgcgctgcccaacgctcttgcgaacgccgccatcgcgcagctggcggcctcgacgcgcggcgccgccgccgcgccccggccgctccacctcgtcgacatcggcgtttcgcacggagtgcagtggccgactttgctggagtccctgacccgcgtgcccgccggacgagcaccgccgtctgtccggctcacggtcgtaggcatcccaccggtgacgacgccattctcggcgtcgcctccggggtacgacttctcaccgcatctcctccggtacgccaagtccattaacctggacctcgagatcagccgcgccgcctcgctagactccgcgcatgccttgtacggtccggacgaggcgctcgttgtgtgcctccagttccggctcggccacgtcacggccgaggagcgaacggacatcctccggagggtcaaagacctcaagccggatctggtggtactctcggagctcgacgtcggcggaggcaacgtcagcaacgcggcgtgcgagttcacggcgaggctagagctgctgtggcggttcttggagtcgacgtcggcggcgttcaaggggagggacggggaggacaggcggctgatggaggccgaggcggggactgccgtggcggttaccgcggcaggggatcatggcagggagggctggcgggagcgcatgacggcggcggggttcgaggaggcgccgttcgacggcgaggcggtggagtccgccaggtcgctgctgaggaagtacgacaacgggtgggagatgtccacggcgcccgcggcgacaggaggcggcggcgcggtggcgctgcggtggaaggggcagccggtgtccttctgctcgctgtggcggccggcgtag

SEQ ID NO: 134 - DNA, 1605 bp, Lolium perenne; identical to SEQ ID NO: 133
  misc_feature 1..1605: note = ORF of protein Nsp1
  source 1..1605: mol_type = unassigned DNA

SEQ ID NO: 135 - DNA, 1701 bp, Arabidopsis thaliana
  misc_feature 1..1701: note = ORF of protein Pds
  source 1..1701: mol_type = unassigned DNA
atggttgtgtttgggaatgtttctgcggcgaatttgccttatcaaaacgggtttttggaggcactttcatctggaggttgtgaactaatgggacatagctttagggttcccacttctcaagcgcttaagacaagaacaaggaggaggagtactgctggtcctttgcaggtagtttgtgtggatattccaaggccagagctagagaacactgtcaatttcttggaagctgctagtttatctgcatccttccgtagtgctcctcgtcctgctaagcctttgaaagttgtaattgctggtgctggattggctggattgtcaactgcaaagtacctggctgatgcaggccacaaacctctgttgcttgaagcaagagatgttcttggtggaaagatagctgcatggaaggatgaagatggggactggtatgagactggtttacatattttcttcggtgcttatccgaatgtgcagaatttatttggagaacttgggatcaatgatcggttgcagtggaaggaacactccatgatttttgctatgccaagtaaacctggagaatttagtagatttgacttcccagatgtcctaccagcacccttaaatggtatttgggctattttgcggaacaacgagatgctgacatggccagagaaaataaagtttgctattggacttttgccagccatggtcggcggtcaggcttatgttgaggcccaagatggtttatcagtcaaagaatggatggaaaagcagggagtacctgagcgcgtgaccgacgaggtgtttattgccatgtcaaaggcgctaaactttataaaccctgatgaactgtcaatgcaatgcattttgatagctttgaaccggtttcttcaggaaaaacatggttccaagatggcattcttggatggtaatcctccggaaaggctttgtatgccagtagtggatcatattcgatcactaggtggggaagtgcaacttaattctaggataaagaaaattgagctcaatgacgatggcacggttaagagtttcttactcactaatggaagcactgtcgaaggagacgcttatgtgtttgccgctccagtcgatatcctgaagctccttttaccagatccctggaaagaaataccgtacttcaagaaattggataaattagttggagtaccagttattaatgttcatatatggtttgatcgaaaactgaagaacacatatgatcacctactctttagcagaagtaaccttctgagcgtgtatgccgacatgtccttaacttgtaaggaatattacgatcctaaccggtcaatgctggagctagtatttgcaccagcagaggaatggatatcacggactgattctgacatcatagatgcaacaatgaaagaacttgagaaactcttccctgatgaaatctcagctgaccaaagcaaagctaaaattctgaagtaccatgtcgttaagactccaagatctgtgtacaagaccatcccaaactgtgaaccatgtcgtcctctacaaagatcacctattgaaggattctacttagctggagattacacaaaacagaagtacttagcttccatggaaggcgctgtcctctctggcaaattctgctctcagtctattgttcaggattacgagctactggctgcgtctggaccaagaaagttgtcggaggcaacagtatcatcatcatga

SEQ ID NO: 136 - DNA, 1899 bp, Arabidopsis thaliana
  misc_feature 1..1899: note = ORF of protein Pen3
  source 1..1899: mol_type = unassigned DNA
atggaaactgatcctgctaactcttcatcgaagtctccagctgtagtatctgagaaagatgtattgattcctgaaccttctgaaaatactgtgggcgttgtacaagatcctgtgagcgctgaaagagaagctcatgaagactctattagtacagaagcttctgttgcaaaggttgatgatactcaaatgcctgctagttctaccggctctgaacctttaagtaaaacagatgatatagttccatgcccaccaggttcctccccacgcgaatcaccgccgtctatattttcttcttctggtctttcttcttgggccaaaagttttaaattccagcagcaagatccaaatcgtacagattctgggatgtcagctttcacaagattcacctctgaacttggtctgcatttgccaacgaaagggtctgaggaggttggtgattctagatcatcaaacacacaagttggtggtgcttttgagtccctcacaaaagctgtggttgattcatctcgtggtgccgttaaagcaatgcaggtgaaagcacgccacattgtctctcagaacaaaagaagataccaggagggggaattcgatttggacatgacatatataactgagaatataatcgcaatggggttcccagctggtgacataagctctggtcttttcggatttttcgagggactatatcgtaatcacatggaagaggtgataaagttttttgaaactcatcataaggataaatataaggtctacaacctttgttcagagagattgtacgatgcttcaagatttgaggggaaggttgcatctttcccatttgatgatcataattgtcctcctatccaattgataccatcgttttgtcaaagtgcatacacatggctgaaggaagatatccaaaatgtagtggtagttcattgtaaagctggaatggcaagaaccggattaatgatttgctgtcttcttctatatctcaagttctttcctacagctgaagaggctattgattactataaccaaaagagatgtcttgatgggaaagctcttgttctcccaagtcagattcgatatgtcaagtactacgaacgtgttcaaaaccaatttgacgggaaagttccacccgagcgaagatgcatgctcagaggatttcgcctaatcaactgcccttactggattagacctgccattacgatctccaaccacactgatatacttttctcaacaaagaaacatcagaaaaccaaggatttggggcctgaagatttttggataaaggcacccaaaaagggagtggttgtctttgcaatacctggtgaagctggtctaacagaattagccggcgacttcaaaatccactttcaagatagcgatggagatttctactgttggctaaatactacactgactgataacagaacaatgctgaaaggcagtgactttgatggctttgacaagagaaaactgcctgcaccgggtttccatgtcgagatcgtaatgatagaacccgacaattcacaaccaacaaagtccaaatctgattccacccaacaacaatctcaaagcagctccagtgctgattccagcaaacttaagtctaatgagaaagacgacgatgtattctctgacagtgatggggaagaggaaggaaactctcagtcttattcaactaacgagaaaaccgcaagctcaatgcacacaactagtaaacctcatcagatcaatgagccaccaaagagagatgacccttccgcaaacagaagtgtcacaagttcatcgtcctcgggtcattataatccaataccaaacaactcattagcagttagtgatatcaaagctattgctgcagatgcatcggtcttctcctttggagatgaggaagaagactatgaaagcgactga

SEQ ID NO: 137 - DNA, 3519 bp, Arabidopsis thaliana
  misc_feature 1..3519: note = ORF of protein Phyb
  source 1..3519: mol_type = unassigned DNA
atggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgcagttgcaagagagatggttaggctcattgagactgcaactgttcctatattcgctgtggatgccggaggctgcatcaatggatggaacgctaagattgcagagttgacaggtctctcagttgaagaagctatggggaagtctctggtttctgatttaatatacaaagagaatgaagcaactgtcaataagcttctttctcgtgctttgagaggggacgaggaaaagaatgtggaggttaagctgaaaactttcagccccgaactacaagggaaagcagtttttgtggttgtgaatgcttgttccagcaaggactacttgaacaacattgtcggcgtttgttttgttggacaagacgttactagtcagaaaatcgtaatggataagttcatcaacatacaaggagattacaaggctattgtacatagcccaaaccctctaatcccgccaatttttgctgctgacgagaacacgtgctgcctggaatggaacatggcgatggaaaagcttacgggttggtctcgcagtgaagtgattgggaaaatgattgtcggggaagtgtttgggagctgttgcatgctaaagggtcctgatgctttaaccaagttcatgattgtattgcataatgcgattggtggccaagatacggataagttccctttcccattctttgaccgcaatgggaagtttgttcaggctctattgactgcaaacaagcgggttagcctcgagggaaaggttattggggctttctgtttcttgcaaatcccgagccctgagctgcagcaagctttagcagtccaacggaggcaggacacagagtgtttcacgaaggcaaaagagttggcttatatttgtcaggtgataaagaatcctttgagcggtatgcgtttcgcaaactcattgttggaggccacagacttgaacgaggaccagaagcagttacttgaaacaagtgtttcttgcgagaaacagatctcaaggatcgtcggggacatggatcttgaaagcattgaagacggttcatttgtgctaaagagggaagagtttttccttggaagtgtcataaacgcgattgtaagtcaagcgatgttcttattaagggacagaggtcttcagctgatccgtgacattcccgaagagatcaaatcaatagaggtttttggagaccagataaggattcaacagctcctggctgagtttctgctgagtataatccggtatgcaccatctcaagagtgggtggagatccatttaagccaactttcaaagcaaatggctgatggattcgccgccatccgcacagaattcagaatggcgtgtccaggtgaaggtctgcctccagagctagtccgagacatgttccatagcagcaggtggacaagccctgaaggtttaggtctaagcgtatgtcgaaagattttaaagctaatgaacggtgaggttcaatacatccgagaatcagaacggtcctatttcctcatcattctggaactccctgtacctcgaaagcgaccattgtcaactgctagtggaagtggtgacatgatgctgatgatgccatattag

SEQ ID NO: 138 - DNA, 1575 bp, Arabidopsis thaliana
  misc_feature 1..1575: note = ORF of protein Pif3
  source 1..1575: mol_type = unassigned DNA
atgcctctgtttgagcttttcaggctcaccaaagctaagcttgaatctgctcaagacaggaacccttctccacctgtagatgaagttgtggagctggtgtgggaaaatggtcagatatcaactcaaagtcagtcaagtagatcgaggaacattcctccaccacaagcaaactcttctagagctagagagattggaaatggctcaaagacgactatggtggacgagatccctatgtcagtgccatcactaatgacgggtttgagtcaagacgatgactttgttccatggttgaatcatcatccctcccttgatggatattgctctgatttcttgcgtgatgtgtcgtctcctgttactgtcaacgagcaagagagtgatatggcggtaaaccaaactgctttcccgttgtttcagagaagaaaggatggcaatgaatcagctcctgctgcttcttcgtcgcagtataacggtttccaatcgcattctctgtatggaagtgatagagctagagatcttcctagccaacaaaccaatccggatcggtttactcagacgcaggaaccactaattactagtaacaagcctagtttggtcaacttttcacatttcttacgccctgcaacttttgcgaagactactaataataaccttcatgacactaaagaaaagagtcctcaaagcccgccaaatgtgtttcagaccagagttcttggagctaaagactctgaagataaggttcttaacgagtctgttgcttctgctacgcctaaagataaccaaaaggcttgcctaatatcagaggactcatgtagaaaagaccaagagagtgaaaaagcagttgtatgttcttctgttggctcgggtaatagtctcgatggcccatccgaaagtccttcactttctttaaagagaaagcattcgaatattcaagacattgactgtcatagtgaagatgtggaagaagaatcaggagatggaagaaaggaagcaggtccatctcgaacgggtttgggttcaaagagaagccgctctgcagaagtgcataatctgtctgaaaggagacggcgtgataggatcaacgagaagatgcgtgccctgcaagaactcattccaaactgtaacaaggtggacaaagcttcgatgctagatgaagccatcgagtatctcaagtcactccaacttcaagtgcagatcatgtcaatggcgtctggttactatctgccaccggcggttatgttcccaccgggtatggggcattacccggcagcagctgctgcaatggcaatgggtatgggaatgccttatgcaatgggcttgcctgatttgagccgtggtggttcatcggttaaccacggaccacagttccaagtctcggggatgcaacaacaaccagtggcgatgggtattccacgtgtctctggtggtggtatctttgccggttcttcgacgattggcaatggctcgactagagatttatctggttctaaagatcaaacaacgacgaataacaacagtaacttgaaaccaataaagagaaaacaggggtcttctgatcagttttgtggatcgtcgtga

SEQ ID NO: 139 - DNA, 1377 bp, Arabidopsis thaliana
  misc_feature 1..1377: note = ORF of protein Pizza
  source 1..1377: mol_type = unassigned DNA
atgcccatgttaatggcgacacgtatcgatataatccaaaagcttaatgtatatccaaggtttcaaaaccatgacaagaagaaactaatcactctctccaatttggaccgtcagtgtcctttactcatgtactctgtcttcttctacaagaataccacaactcgtgactttgactccgtcttctccaacctgaagctcgggctggaggagactatgtctgtgtggtatcccgcggcagggagactgggtttggacggaggtggctgcaagctcaacatccggtgtaacgatggtggcgcagtcatggtggaggcggtggcgacaggtgtcaagttgtccgagcttggtgatttgactcagtacaatgagttttatgagaatttagtttacaagccttccttggatggtgatttctctgtgatgcctcttgttgttgctcaggtgacaagatttgcatgtggaggttactcaattggaatcggtacaagccactctctatttgatggaatctcagcttacgaattcattcacgcgtgggcctccaactctcacattcacaacaaatccaacagcaagattactaataaaaaggaagatgtggtcatcaaaccggttcatgatcgacgaaatctactggttaaccgggatgctgtccgagaaaccaatgctgcagccatttgtcatctgtaccagttgatcaaacaggcgatgatgacctatcaggagcaaaaccgtaacttagagttaccagactctggttttgtgatcaaaacgttcgagcttaatggcgatgcgatagaaagcatgaagaagaaatcactagaagggttcatgtgctcctcctttgagtttcttgctgctcatttgtggaaggcaagaacaagggctttagggttgaggagagacgccatggtgtgtttacaattcgcagtggacataaggaaaagaacggagacaccgctgccagaagggttttccggcaacgcatacgtgcttgcctcggtggcatccaccgccagagaattacttgaagagctaacactcgagtcaatagtcaacaagatcagagaagccaagaaatcaattgaccaaggttacataaactcttacatggaagcactcggaggtagtaatgacggaaatctccctcctctcaaagagctaaccctaatctccgactggacaaaaatgccatttcacaatgttggctttggcaacggcggcgagccagcggattacatggccccactgtgtccaccggtgccacaagttgcttatttcatgaagaaccctaaagatgccaaaggtgttcttgtgaggattggcttggacccacgagatgttaatggcttttcaaatcatttccttgattgctaa

SEQ ID NO: 140 - DNA, 1521 bp, Arabidopsis thaliana
  misc_feature 1..1521: note = ORF of protein Ppox1
  source 1..1521: mol_type = unassigned DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctctcaggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaactacattggcgggtctacaaacaccggaattctgtccaagtctgaaggtgagttagtggaagcatttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 141 - DNA, 1527 bp, Arabidopsis thaliana
  misc_feature 1..1527: note = ORF of protein Ppox2
  source 1..1527: mol_type = unassigned DNA
atggcgtctggagcagtagcagatcatcaaattgaagcggtttcaggaaaaagagtcgcagtcgtaggtgcaggtgtaagtggacttgcggcggcttacaagttgaaatcgaggggtttgaatgtgactgtgtttgaagctgatggaagagtaggtgggaagttgagaagtgttatgcaaaatggtttgatttgggatgaaggagcaaacaccatgactgaggctgagccagaagttgggagtttacttgatgatcttgggcttcgtgagaaacaacaatttccaatttcacagaaaaagcggtatattgtgcggaatggtgtacctgtgatgctacctaccaatcccatagagctggtcacaagtagtgtgctctctacccaatctaagtttcaaatcttgttggaaccatttttatggaagaaaaagtcctcaaaagtctcagatgcatctgctgaagaaagtgtaagcgagttctttcaacgccattttggacaagaggttgttgactatctcatcgacccttttgttggtggaacaagtgctgcggaccctgattccctttcaatgaagcattctttcccagatctctggaatgtagagaaaagttttggctctattatagtcggtgcaatcagaacaaagtttgctgctaaaggtggtaaaagtagagacacaaagagttctcctggcacaaaaaagggttcgcgtgggtcattctcttttaaggggggaatgcagattcttcctgatacgttgtgcaaaagtctctcacatgatgagatcaatttagactccaaggtactctctttgtcttacaattctggatcaagacaggagaactggtcattatcttgtgtttcgcataatgaaacgcagagacaaaacccccattatgatgctgtaattatgacggctcctctgtgcaatgtgaaggagatgaaggttatgaaaggaggacaaccctttcagctaaactttctccccgagattaattacatgcccctctcggttttaatcaccacattcacaaaggagaaagtaaagagacctcttgaaggctttggggtactcattccatctaaggagcaaaagcatggtttcaaaactctaggtacacttttttcatcaatgatgtttccagatcgttcccctagtgacgttcatctatatacaacttttattggtgggagtaggaaccaggaactagccaaagcttccactgacgaattaaaacaagttgtgacttctgaccttcagcgactgttgggggttgaaggtgaacccgtgtctgtcaaccattactattggaggaaagcattcccgttgtatgacagcagctatgactcagtcatggaagcaattgacaagatggagaatgatctacctgggttcttctatgcaggtaatcatcgaggggggctctctgttgggaaatcaatagcatcaggttgcaaagcagctgaccttgtgatctcatacctggagtcttgctcaaatgacaagaaaccaaatgacagcttataa

SEQ ID NO: 142 - DNA, 2472 bp, Arabidopsis thaliana
  misc_feature 1..2472: note = ORF of protein Prp39
  source 1..2472: mol_type = unassigned DNA
atgggtgacagcgaggccatggtttccgagggttatacttctgctccatatggagactataatgcttctgctgctactgtggaatcgaccgggcaagagactgcaccaattgttgatgcatcacactcggtcaacaatgattctttggtcaatggtactgcgccagttgagaacggaagtgcaacagataatgtggctgtgactgctccagcagcggagcatggagacaatactggctctacactctcaacggaagaggagcgcttgtggaatattgtaagggcaaattctttagagtttaatgcttggactgccttgattgatgagacggagaggatagcgcaggacaatatagcaaaaatccggaaggtctatgatgctttcttagctgaatttcctctgtgttatggctattggaaaaagtttgccgatcatgaggctcgggtgggggcaatggacaaagtcgtggaggtttatgaaagagcagtgctgggagtgacatattcagtggatatctggttgcattattgcacttttgccatcaatacatatggagatccagaaacgatcagaaggctttttgaacgagctttggtttacgttggaactgattttctttcctctccgttgtgggacaaatacattgagtatgagtacatgcagcaggactggagccgagttgccttgatttacaccagaatattggagaatccaattcaaaatctggatagatatttcagcagttttaaggagctagctgaaacacggcctctgtcggaactaaggagtgctgaggaatccgcagctgctgctgttgctgttgctggtgatgcttctgaaagtgcagcatctgagtccggtgaaaaggcagatgaaggacgatctcaagttgatggttccaccgaacaatcccctaaattggaaagtgctagttcaactgaacctgaggagttgaagaagtatgtaggcatcagggaagccatgtacataaaatcgaaagagtttgaatctaaaatcattggttatgaaatggctataaggaggccctatttccatgtgcgtcctctgaatgtcgcagaactggagaattggcacaactatctggatttcattgagagggatggagacttcaataagttaagtagtatctggtgcatcatatgcttgattgggtttccattggatcaagcaacttttaaatgggaaataactgagaccaaagcatgtgcctcgatttgttccaatgttattaatgcaggtgtcttcctaacattctgtctgtcaggcaaggagggtccctctgtggtcaagctgtatgaaagatgtgtggttacctgtgcaaattacccagaatactggattcgttatgtgacaaacatggaagcaagtggaagtgcggaccttgcagaaaatgcccttgctcgagcaactcaagtctttgtcaagaaacaaccagagattcacctatttgctgctcgattaaaagagcagaatggagatatagctggtgctagagctgcataccaattagtgcactctgaaatttctcctggacttcttgaagcagtaatcaagcatgcaaatatggaataccgactaggtaatctggatgatgctttctctttgtatgagcaagtgattgctgttgaaaaggggaaagaacattccacaatactgccactgctgtatgcgcagtattcaaggttttcatacttggtctccagggatgctgagaaagctaggaggattattgtcgaagcacttgaccatgtacaaccgtcaaaacctctcatggaagcactgattcattttgaggcgattcagccaccaccaagagagattgattaccttgagccacttgtagagaaagttataaagccagatgcagatgcccaaaacattgcaagttccactgagagggaagagctatccttaatatatatagagttcctgggtatttttggagatgtgaagtccattaaaaaagcggaagatcaacatgttaaactgttttatcctcatcggagcacgtcggagctgaaaaagcgtagcgcagatgattttctcgcatcagataggacgaaaatggcaaaaacttacaacggcactccacctgctcagccagtatccaatgcatatccaaatgctcaggctcaatggtctggtggttatgctgcgcagcctcagacttggccaccagcacaagctgctcctgctcaaccacagcaatggaaccctgcctacggtcaacaggctgcttatggtgcatatgggggatatcctgctggctataccgctccacaagcaccaacacctgtgccacaggccgcagcttatggagcgtatcctgctcagacatacccaacgcagagttatgcacctccagttgcagcagcagcaccagcggctgcaccggtgcagcaaccggctgctgctgttgctcctcaagcgtactacaacacgtactactga

SEQ ID NO: 143 - DNA, 1062 bp, Arabidopsis thaliana
  misc_feature 1..1062: note = ORF of protein PsbA
  source 1..1062: mol_type = unassigned DNA
atgactgcaattttagagagacgcgaaagcgaaagcctatggggtcgcttctgtaactggataactagcactgaaaaccgtctttacattggatggtttggtgttttgatgatccctaccttattgaccgcaacttctgtttttattatcgcattcattgctgctcctccagtagatattgatggtattcgtgaacctgtttctggatctcttctttacggaaacaatattatttccggtgccattattcctacttctgcagctattggattgcatttttacccaatctgggaagctgcatccgttgatgaatggctatacaacggcggtccttatgaactaattgttctacactttttacttggtgtagcttgttatatgggtcgtgagtgggaacttagtttccgtctgggtatgcgtccttggattgctgttgcatattcagctcctgttgcagctgcgactgctgttttcttgatctatccaattggtcagggaagtttttctgatggtatgcctctaggaatctctggtactttcaactttatgattgtattccaggctgagcacaacattcttatgcacccatttcacatgttaggtgtagctggtgtattcggcggctccctttttagtgctatgcatggttccttggtaacttctagtttgatcagggaaaccacagaaaatgaatctgctaatgaaggttacagattcgggcaagaagaagaaacttacaacattgtagctgctcacggttattttggccgattgattttccaatatgctagtttcaacaattctcgttctttacatttcttcttagcggcttggccggtagtaggtatttggtttactgctttaggtattagtactatggctttcaacctaaatggtttcaatttcaaccaatcagtagttgatagtcaaggacgtgttattaatacttgggctgatattattaaccgtgctaaccttggtatggaagttatgcatgaacgtaatgctcacaacttccctctagacctagctgctgttgaggctccatctacaaatggataa

SEQ ID NO: 144 - DNA, 3027 bp, Arabidopsis thaliana
  misc_feature 1..3027: note = ORF of protein Pskr1
  source 1..3027: mol_type = unassigned DNA
atgcgtgttcatcgtttttgtgtgatcgtcatcttcctcacagagttactatgtttcttctattcctcggaatctcagaccacctccaggtgccatccacatgacctcgaagccttacgtgacttcatagcacatctcgaaccaaaaccagatggttggatcaattcttcttcttctacagactgctgcaactggaccggaatcacctgcaattcaaacaacaccggaagagttattagattggagcttgggaacaaaaagctgtcggggaagttgtctgaatctctcgggaagctagatgagattagggttcttaatctctctcgaaacttcatcaaagattcgatccctctttcgattttcaacttgaagaatctacaaactcttgatttgagctctaatgatctctccggcggaatcccaacaagtataaatctcccagctctgcaaagttttgatctttcttcaaataaattcaatgggtcgcttccgtctcatatctgccataactctactcaaattagggttgtgaaacttgcggtgaactacttcgccggaaacttcacttccgggtttgggaaatgtgtcttgcttgagcatctctgtcttggtatgaacgatcttactggtaacatccctgaggatttgtttcatctcaaaagattgaatcttttagggattcaagagaatcgtctctctggttcgttgagtcgtgagattaggaatctctcaagtcttgttcgtcttgatgtttcttggaatttgttttccggtgaaatccctgatgtgttcgacgaattgcctcagttaaagtttttcttaggtcagaccaatggattcattggaggaatacctaaatcgttggcgaattcaccgagtttgaatctgcttaacttgaggaacaattctttatcgggtcgtttgatgttgaattgtacggcgatgattgctttgaactctcttgatttaggtaccaatagattcaatgggaggttacctgagaatctaccggattgcaagcggttaaagaacgttaacctcgcgaggaacaccttccatggacaagtaccagagagtttcaagaacttcgagagcttatcttacttctcgttatcgaattcgagtttggctaatatctcttcagcgcttgggatacttcagcattgcaagaacttgacgactttggttcttacattgaatttccatggagaggctttacccgatgattcaagtcttcatttcgagaagcttaaggtgcttgtagtggcgaattgtaggcttactggttcgatgccgaggtggttaagctcgagtaatgaacttcagttgttggatctttcttggaaccgtttaaccggcgctatcccgagctggattggtgacttcaaggctctgttctacttggatttatctaacaactcgtttacaggagagatccctaagagcttaactaagttagagagtctcactagccgtaatatctcagtcaatgagccatctcctgatttcccgttctttatgaaaagaaacgagagcgcgagagcgttgcaatacaatcagattttcgggttcccgccaacgattgagcttggtcataacaatctctctggacctatttgggaggagtttggtaatctgaagaagcttcatgtgtttgatttgaaatggaatgcattatctggatcaatacctagctcgctttctggtatgacgagcttggaagctcttgatctctctaataaccgtctttcgggttcgatcccggtttctctgcaacagctctcgtttctgtcgaagttcagtgttgcttataacaatctctcgggagtaataccttccggtggtcagtttcagacgtttccaaactcgagctttgagagtaaccatctctgcggggaacacagattcccctgttctgaaggtactgagagtgcattgatcaaacggtcaagaagaagcagaggaggtgacattggaatggcgattgggatagcgtttggttcggtttttcttttgactcttctctcgttgattgtgttgcgtgctcgtagacggtcaggagaagttgatccggagatagaagaatccgagagcatgaatcgtaaagaactcggagagattggatctaagcttgtggttttgtttcagagcaatgataaagagctctcttatgatgaccttttggactcaacaaatagttttgatcaagctaacatcattggctgtggcgggtttggtatggtttacaaagcaacgttaccagacggtaagaaagttgcgatcaagaagttatccggtgattgcggtcaaatcgaaagagaattcgaagcagaagttgaaacactctcaagagcacagcatccaaatcttgttcttctccgaggattctgtttctacaaaaacgaccggcttttaatctactcgtatatggaaaacggaagcttagactattggctacacgagcgtaacgacggtccagcgttgttgaagtggaaaacacgtcttagaatcgctcaaggtgctgcaaaagggttactttacttgcatgaagggtgtgatcctcatatcttacaccgcgatattaaatcgagtaatattcttctcgacgagaatttcaactctcatttagcggatttcggactcgcaaggctgatgagtccttacgagacgcatgtaagtactgatttggttggaactttaggttacattcctccggaatacgggcaagcttcggttgctacttacaaaggcgatgtgtatagtttcggagttgtgcttctcgagcttttaaccgataaaagaccggtggatatgtgtaaaccgaaagggtgtagggatctgatctcgtgggtcgtcaagatgaagcatgagagtcgagcaagcgaggttttcgatccgttaatatacagtaaagagaatgataaagagatgtttcgggttctcgagattgcttgtttatgtttaagcgaaaacccgaaacagaggccaacgactcaacagttagtctcttggcttgatgatgtctag

SEQ ID NO: 145 - DNA, 1389 bp, Arabidopsis thaliana
  misc_feature 1..1389: note = ORF of protein Rd21
  source 1..1389: mol_type = unassigned DNA
atggggttccttaagccaaccatggcgattctttttctagcgatggtcgccgtttcatcagccgtggacatgtcaatcatctcctacgacgagaaacatggcgtctccaccaccggtggccgtagcgaagccgaggttatgagtatctacgaggcatggttggtgaaacacggcaaggctcagagccagaactctcttgttgagaaagatcgacggttcgagatctttaaagacaatcttcgtttcgtcgatgaacataacgagaagaatcttagttatagattgggtttgactcgttttgcggatcttactaacgatgagtatagatccaagtatcttggagctaagatggagaagaaaggtgagagaaggactagcctacggtacgaggctcgtgtcggtgatgagctaccggagtctattgactggaggaagaaaggagccgtggctgaggtcaaagatcagggtggttgcgggagttgttgggcgttttcaaccattggagcagtggagggaataaaccagatcgtaaccggagacctaataaccttgtctgaacaagagttggtcgattgtgacacttcatacaacgaaggttgtaacggaggtcttatggactatgcttttgagttcattatcaagaatggtggaatcgatacagacaaagattatccttacaagggtgttgatggaacttgtgaccagatcaggaaaaacgctaaagttgtcactatcgattcatatgaggatgttccaacttacagcgaggaatcgttgaagaaagcagttgctcatcaacccattagcattgccattgaggctggtggtcgtgcgttccagctctatgattcgggtatatttgatggaagttgtggaacacaactagaccacggagttgtggcggttggatacggaactgagaacggcaaagattactggattgtgagaaactcatggggtaaaagctggggagagagtggatacctaaggatggcgcgtaacattgcgtcttcatcaggaaaatgtggaatcgcgattgaaccttcatacccgataaagaatggcgaaaacccgccaaacccgggaccttcacctccatctcccatcaagcctccaacccaatgtgacagttactacacttgtcctgagagcaacacttgttgttgtctgtttgagtatggcaagtattgctttgcttggggatgttgcccactagaagcagccacttgctgtgatgacaactatagttgctgccctcacgagtacccggtttgtgaccttgatcaaggaacctgtttattgagcaagaacagtccatttagtgttaaggccttaaagcgtaaacccgcaacgccattctggtcacaaggcagaaagaacattgcctaa

SEQ ID NO: 146 - DNA, 906 bp, Arabidopsis thaliana
  misc_feature 1..906: note = ORF of protein Ring1
  source 1..906: mol_type = unassigned DNA
atggcgtttaatcacagaaaaatgcttctttcatgtctacagtttaaagatttacgcttttgtttccggcaataccctcccccgccaccaccaccaccgccaccgagggagctctctctcctactccccacctcgatctgcgtcgtcggctcaatcatactctttctatttctagtcttcttcctctacctccacatcacgcaacaacggcggatttcagccgccagcgtaactcccggtgacacaaaccaacaggaagatgaggacgaaacagaggagagagatttctccgactttcatcacgtttggcaaatcccaaccgtcggtctccaccgttccgcgatcaactcgatcacagtcgttggattcaaaaagggagaagggataatcgacggcacagaatgctctgtctgcttaaacgagtttgaagaagacgagtcgctgagactgttaccaaagtgtagccatgcctttcatctaaattgcatcgacacttggttgctctctcacaagaactgtcctttatgtcgtgcccctgtacttcttatcactgaaccacctcatcaagaaacagagacgaatcatcaaccagattctgaatcgagcaatgacctccgtggaagacaagacagttccagatctagaagaaaccataacatttttcttccaagggcacaaagtgatttggcaaactactgtggctcaggaagagtagaaaacgtgagaagatcgttttcaattggaggaagcttatcactttgcgatggtattaacaatgctactagaagtggaagacagttctataccagcttcagtgctaacttgttctcatcatcaagaagagtaaggaacgaacaaccgattccacaaaaccaaatgccatccgtaactggtaacaccagttaa

SEQ ID NO: 147 - DNA, 4182 bp, Arabidopsis thaliana
  misc_feature 1..4182: note = ORF of protein Ros1
  source 1..4182: mol_type = unassigned DNA
atggagaaacagaggagagaagaaagcagctttcaacaacctccatggattcctcagacacccatgaagccattttcaccgatctgcccatacacggtggaggatcaatatcatagcagtcaattggaggaaaggagatttgttgggaacaaggatatgagtggtcttgatcacttgtcttttggggatttgcttgctctagctaacactgcatccctcatattctctggtcagactccaatacctacaagaaacacagaggttatgcaaaaaggtactgaagaagtggagagtttgagctcagtgagtaacaatgttgctgaacagatcctcaagactcctgaaaaacctaagaggaagaagcatcggccaaaggttcgtagagaagctaaacccaagagggagcctaaaccacgagctccgaggaagtctgttgtcaccgatggtcaagaaagcaaaacaccaaagaggaaatatgtgcggaagaaggttgaagtcagtaaggatcaagacgctactccggttgaatcatcagcagctgttgaaacttcaactcgtcctaagaggctctgtagacgagtcttggattttgaagccgaaaatggagaaaaccagaccaacggtgacattagagaagcaggtgagatggaatcagctcttcaagagaagcagttagattctgggaatcaagagttaaaagattgccttctttcggctcctagcacgcccaagagaaagcgcagccaaggtaaaagaaagggagttcaaccaaagaaaaatggcagtaatctagaagaagtcgatatttcgatggcgcaagctgcaaagagaagacaaggaccaacttgttgcgacatgaatctatcagggattcagtatgatgagcaatgtgactaccagaaaatgcattggttgtattccccaaacttgcaacagggagggatgagatatgatgccatttgcagcaaagtattctctggacaacagcacaattatgtttctgcctttcacgctacgtgctacagttccacatctcagctcagtgctaatagagtcctaaccgttgaagaaagacgagaaggtatctttcaaggaaggcaagagtctgagctaaatgttctctcggataagatagacacgccgatcaagaagaaaacaacaggccatgctcgattccggaatttgtcttcaatgaataaacttgtggaagttcctgagcatttaacctcaggatattgtagcaagccacagcaaaataataagattcttgttgatacgcgggtgactgtgagcaaaaagaagccaaccaagtctgagaaatcacaaaccaaacagaaaaatcttcttccgaatctttgccgttttccaccttcatttactggtctttctccagatgaactttggaaacgacgtaactcgatcgaaacaatcagtgagctattgcgtctattagacatcaacagggagcattctgaaactgctctcgttccttacacaatgaatagccagattgtactctttggtggtggcgctggagcaattgtgcctgtaactcctgttaaaaaaccacgcccacgaccaaaggttgatctagacgatgagacagacagagtgtggaaactgctattggagaatattaatagcgaaggtgttgacggatcagacgagcagaaggcgaaatggtgggaggaagaacgtaatgtgtttcgaggacgagctgactcatttattgcaaggatgcaccttgtacaaggggatcgacgttttacgccttggaagggatccgtcgtggattctgttgttggagtatttctcactcaaaatgtttcagaccatctctcaagttcggctttcatgtcgttggcttcccagttccctgtcccttttgtaccgagcagtaactttgacgctggaacaagctcgatgccttctattcaaataacgtacttggactcagaggaaacgatgtcaagcccacccgatcacaatcacagttctgttactttgaaaaatacacagcctgatgaggagaaggattatgtacctagcaatgaaacctccagaagcagtagtgagattgccatctcagcccatgaatcagttgacaaaaccacggattcaaaggagtatgttgattcagatcgaaaaggctcaagtgtagaggttgataagacggatgagaagtgtcgtgtcctgaacctgtttccatctgaagattctgcacttacatgtcaacattcgatggtgtctgatgctcctcaaaatacagagagagcaggatcaagctcagagatcgacttagaaggagagtatcgtacttcctttatgaagctcctacagggggtacaagtctctctagaagattccaatcaagtatcaccaaatatgtctccgggtgattgtagctcagaaattaagggtttccagtcaatgaaagagcccacaaaatcctctgttgatagtagtgaacctggttgttgctctcagcaagatggggatgttttgagttgtcagaaacctaccttaaaagaaaaagggaaaaaggttttgaaggaggaaaaaaaagcgtttgactgggattgtttaagaagagaagcccaagctagagcaggaattagagaaaaaacaagaagtacaatggacaccgtggattggaaggcaatacgagcagcagatgttaaggaagttgctgaaacaatcaagagtcgcgggatgaaccataaacttgcagaacgtatacagggcttccttgatcgactggtaaatgaccatggaagtatcgatcttgaatggttgagagatgttccaccagataaagcaaaagaatatcttctgagctttaacggattgggactgaaaagtgtggagtgtgtgcggcttctaacacttcaccatcttgcctttccagttgatacaaatgttgggcgcatagccgtcagacttggatgggtgccccttcagccgctcccagagtcacttcagttgcatcttctggaaatgtatcctatgcttgaatctattcaaaagtatctttggccccgtctctgcaaactcgaccaaaaaacattgtatgagttgcactaccagatgattacttttggaaaggtcttttgcacaaagagcaaacctaattgcaatgcatgtccgatgaaaggagaatgcagacattttgccagtgcgtttgcaagtgcaaggcttgctttaccaagtacagagaaaggtatggggacacctgataaaaaccctttgcctctacacctgccagagccattccagagagagcaagggtctgaagtagtacagcactcagaaccagcaaaaaaggtcacatgttgtgaaccaatcatcgaagagcctgcttcaccggagccagaaaccgcagaagtatcaatagctgacatagaggaggcgttttttgaggatccagaagaaattcctaccatcaggctaaacatggatgcatttaccagtaacttgaagaagataatggaacacaacaaggaacttcaagacggaaacatgtccagcgctttagttgcacttactgctgaaactgcttctcttccaatgcctaagctcaagaatatcagccagttaaggacagaacaccgagtttacgaacttccagacgagcatcctcttctagctcagttggaaaagagagaacctgatgatccatgttcttatttgcttgctatatggacgccaggtgagacggctgattctattcaaccgtctgttagtacgtgcatattccaagcaaatggtatgctttgtgacgaggagacttgtttctcctgcaacagcatcaaggagactagatctcaaattgtgagagggacaattttgattccttgtagaacagcgatgaggggtagttttcctctaaatggaacgtactttcaagtaaatgaggtgtttgcggatcatgcatccagcctaaacccaatcaatgtcccaagggaattgatatgggaattacctcgaagaacggtctattttggtacctctgttcctacgatattcaaaggtttatcaactgagaagatacaggcttgcttttggaaagggtacgtatgtgtacgtggatttgatcgaaagacgaggggaccgaagcctttgattgcaagattgcacttcccggcgagcaaactgaagggacaacaagctaacctcgcctaa

SEQ ID NO: 148 - DNA, 1200 bp, Arabidopsis thaliana
  misc_feature 1..1200: note = ORF of protein Rpt4a
  source 1..1200: mol_type = unassigned DNA
atgaccgacgtcgatgaatccgttagacgccgtacagccgccgtttccgagtaccggaaaaagctcctccaacataaggaactcgaatcccgtgttcgaacagctagggagaacttaagaggagctaaaaaggaattcaacaaaaccgaggatgatctcaagtctcttcaaagtgttgggcagattattggagaagtgcttcgacccctggataatgaaagattgattgtgaaagcgagcagtggtccacgttatgtggtgggttgccgaagcaaagtggataaggaaaagctaacctccggaactcgagttgttctcgatatgacaacactaacaattatgcgagcccttccccgagaagttgatcctgttgtttataacatgcttcatgaagaccctggcaacattagctactccgctgtcggagggttaggtgatcagatcagagaactcagggaatctattgagctccctctcatgaatcctgagcttttcctcagagtagggatcaaacctccaaagggtgtccttctgtatggacctccaggtactggaaaaactctactagccagggctattgccagcaacatcgatgctaacttcttgaaggttgtatccagtgcaatcatagacaagtacattggagaaagtgctaggctgattcgggagatgtttaactatgcccgtgaacatcagccttgtatcatattcatggatgaaatcgatgctattggtgggcgtcgttttagtgagggaactagtgctgatcgtgaaattcaaagaacgcttatggagttactcaaccagcttgatgggtttgacaacctcggaaaggtgaaaatgataatggccacaaacaggcctgatgtgcttgatcctgcgcttctccgtccgggaaggttagatagaaagattgagatccctttgccaaacgagcagtcaagaatggatattcttaagatccatgcagctggtatcgccaaacatggtgaaatcgattacgaggctattgtcaaacttgcagagggattcaatggtgctgatctgcgtaacatatgcacggaagctggaatgtttgcaatccgagcagagcgggattatgtgatccatgaagatttcatgaaggcggtgagaaagctgagtgaggccaagaaactggagtccagttcgcactataacgctgattttggaaaagagtga

SEQ ID NO: 149 - DNA, 3804 bp, Arabidopsis thaliana
  misc_feature 1..3804: note = ORF of protein Sfr6
  source 1..3804: mol_type = unassigned DNA
atgaatcagcaaaacccagaagaagaagtttctttggttaataatagcggtggtggaggaatcatcgaagctccagctatagtggaggagaaagaggaagaaggattacagcagaagcaggaagagactattgagtctaccgatccgattctcgtcgtcgttgaggagaaattgttagagaaatcagtagacggagagaaagaagacgataatagtagtagtagtaacatggagattgatcctgtgagtccggctacagttttctgtgttaagcttaagcagcccaattccaatttgcttcataagatgagtgttcctgaattgtgccgtaacttcagtgctgttgcgtggtgtggcaaattgaatgctattgcttgtgcttccgagacctgtgccaggattccaagctccaaggcaaatacacctttttggataccaatacatatcttgatacctgagcgccctactgagtgtgcggtgtttaatgttgtggcagactctcctcgtgattctgtccaatttatcgaatggtctcccacttcttgtcctcgtgcgttactcattgctaattttcatggacgtataactatctggacgcagcctactcagggttcggctaatttagtgcacgacgctacctcctggcagtgtgagcatgaatggcgtcaggacattgctgttgttacaaagtggctgacaggggcttccccatataggtggttgtcctccaagccaagttctggtacaaatgcaaagtcaactttcgaggagaaatttctctcgcagagctctgaaagctcagctcggtggcccaactttctctgtgtatgctctgttttctcatccggctctgttcaaattcattggtcccagtggccttctaaccagggaagcactgcaccaaagtggtttagtacaaagaaaggtcttttaggtgcagggccaagtggaattatggctgctgatgctattataacagacagtggtgccatgcatgtagcaggagttccaattgtaaacccttcaacaattgtagtatgggaggtgacccctggccctggaaatggactccaggcgactccaaaaatctctacaggcagtcgtgtgccaccatcccttagttcttcttcttggacaggttttgctcctttagctgcgtacttgtttagctggcaagaatacttaatatccgagataaagcaagggaagaagccctcagatcaagattccagtgatgctatatcgctaagttgctcaccggtttccaatttttctgcttatgtaagtccagaggctgcagctcagtctgcagcaaccacaacatggggatctggtgttaccgctgttgcttttgatccaactcgtggtggttcagtgatagcagttgttatagttgaagggcagtacatgtctccgtatgatccagatgaaggtccttcaatcacaggctggagagtacagcgctgggaatcaagtgttcaacctgttgtactgcatcagatatttggaaacccaacttcaaattttggaggacaggtccccacgcaaactgtctgggtatccagagtggatatgagcataccacctactaaagattttaagaatcatcaagtagctgcagcaggaccaagtgtggatgcaccaaaggagcctgattctggtgatgagaaggctaacaaggttgtatttgatccttttgatttgccaagtgatattcggacacttgcacggattgtctattctgctcatggtggtgaaattgcgattgcttttcttcgtggtggagttcatatcttttctggtccaactttttcacctgttgaaaactatcaaataaatgttggatctgcaattgctgcacccgcattttcaccaacaagctgttgttcggcttctgtatggcatgatgctgctaaggactgcgcaatgttgaaaatcatccgtgttcttcctcctgctcttccgcgtaaccaatcaaaggttgatcaatcaacatgggagcgggcgatcgctgagagattctggtggagtcttttggtcggagttgattggtgggatgcagttggctgcacacagagtgctgcagaggatgggatagtttcattgaacagcgtgattgctgtcatggatgctgattttcactcccttccttcaacacagcacagacaacaatatggccctaacctagataggatcaaatgtcggttacttgaaggaaccaatgctcaagaggttcgtgccatggttttagatatgcaagcaaggttgttgttggacatgcttggaaaaggtattgaatcagctcttgtgaatccttcggcgttggtttttgagccatggcgagtagatggggagacaataacaggcatcaatccggaggcaatggctgttgatcctgctcttgtttccagtattcaggcttatgtggatgctgttcttgatcttgcttctcatttcatcacacgtttaaggcgttatgcgagtttttgtcggactcttgcaagccatgctgcttctgctggaactggcagtaatcgcaacaatgttaccagtcccacacaaaatgcatcatctcctgcaacacctcagggtcaacctactactactactactactactgctacgacaaactccagcggaagctcacatgtgcaagcttggatgcagggggccatagcgaaaattagtagctcgaatgatggatccaactctactgcaagcccaatcagtggttcacctaccttcatgccaataagcatcaatacaggaacatttccaggaacacctgctgttcggctcattggggattgtcatttccttcatcggttatgccagctgttgctcttctgttttctccagcggtcttcacgatttccacagcgaaatgctgatgttagttcacaaaaacttcaaacgggggctaccagcaaattggaagaagtcaactctgctaaaccaacccctgccttgaacaggatagaggacgcccagggattccggggtgcccagttgggtactggagtgaaagggattgatgaaaattctgctcgtacaacaaagatgggttctgggaatgccggtcaaggatatacttatgaggaggtgagagttcttttccatatactaatggatctctgcaagcgaacatctggtcttgcgcatcccttacctggctctcaggtaggtagtggaaacattcaagttcgactgcattatattgatggaaattacactgtgttacccgaggtggtagaagcggctcttggaccacatatgcagaacatgcctcgcccaagaggagctgatgctgctggtcttctacttcgggagttagagcttcatccgccttccgaagaatggcatagaagaaatttatttggtggtcccgggtcagagcctgaggatatgatcttgacagacgatgtttccaagctgagtaattccttagatctgcctgatacaaacttttccggaatatgtgatggatacaacagagtccatagtctttggccaagaaaacgcaggatgtctgaaagagatgcagcttttggttcaaatacttctgtgggtttgggtgcatatcttgggatcatgggttctcgtagggatgttgtgaccgcgacatggaaaactggtcttgaaggagtttggtacaagtgcataagatgcctaaggcagacatctgcatttgcttcaccaggtgccactaagcagccaaatccgaatgaacgagaaacctggtggacaagtcgttgggtttattgctgccccatgtgtggtggaacgtgggtccgtgttgtatag

SEQ ID NO: 150 - DNA, 1596 bp, Arabidopsis thaliana
  misc_feature 1..1596: note = ORF of protein Shr
  source 1..1596: mol_type = unassigned DNA
atggatactctctttagactagtcagtctccaacaacaacaacaatccgatagtatcattacaaatcaatcttcgttaagcagaacttccaccaccactactggctctccacaaactgcttatcactacaactttccacaaaacgacgtcgtcgaagaatgcttcaactttttcatggatgaagaagacctttcctcttcttcttctcaccacaaccatcacaaccacaacaatcctaatacttactactctcctttcactactcccacccaataccatcccgccacatcatcaaccccttcctccaccgccgcagccgcagctttagcctcgccttactcctcctccggccaccataatgacccttccgcgttctccatacctcaaactcctccgtccttcgacttctcagccaatgccaagtgggcagactcggtccttcttgaagcggcacgtgccttctccgacaaagacactgcacgtgcgcaacaaatcctatggacgctcaacgagctctcttctccgtacggagacaccgagcaaaaactggcttcttacttcctccaagctctcttcaaccgcatgaccggttcaggcgaacgatgctaccgaaccatggtaacagctgcagccacagagaagacttgctccttcgagtcaacgcgaaaaactgtactaaagttccaagaagttagcccctgggccacgtttggacacgtggcggcaaacggagcaatcttggaagcagtagacggagaggcaaagatccacatcgttgacataagctccacgttttgcactcaatggccgactcttctagaagctttagccacaagatcagacgacacgcctcacctaaggctaaccacagttgtcgtggccaacaagtttgtcaacgatcaaacggcgtcgcatcggatgatgaaagagatcggaaaccgaatggagaaattcgctaggcttatgggagttcctttcaaatttaacattattcatcacgttggagatttatctgagtttgatctcaacgaactcgacgttaaaccagacgaagtcttggccattaactgcgtaggcgcgatgcatgggatcgcttcacgtggaagccctagagacgctgtgatatcgagtttccgacggttaagaccgaggattgtgacggtcgtagaagaagaagctgatcttgtcggagaagaagaaggtggctttgatgatgagttcttgagagggtttggagaatgtttacgatggtttagggtttgcttcgagtcatgggaagagagttttccaaggacgagcaacgagaggttgatgctagagcgtgcagcgggacgtgcgatcgttgatcttgtggcttgtgagccgtcggattccacggagaggcgagagacagcgaggaagtggtcgaggaggatgaggaatagtgggtttggagcggtggggtatagtgatgaggtggcggatgatgtcagagctttgttgaggagatataaagaaggtgtttggtcgatggtacagtgtcctgatgccgccggaatattcctttgttggagagatcagccggtggtttgggctagtgcgtggcggccaacgtaa

SEQ ID NO: 151 - DNA, 870 bp, Arabidopsis thaliana
  misc_feature 1..870: note = ORF of protein Shy2
  source 1..870: mol_type = unassigned DNA
atgggagaaaaagaaagtgtccccacaaatgtccccaagaagatgggacacacttttgcctcaaagttgctgttactgctaccgacaagcttagctttttttcctttgtccttaattcagaaaacagtttcttctctctctaccagtatctatctttattctcctttaacttgtataaaacactcagcttcctcgaagcctctcatcttcatcatcagcagcttctctatatctctcctctctttcaaggataataaccaaaagcttttctttttatcttctcctgcaattcttgaagaaatggatgagtttgttaacctcaaggaaacagagctgaggctgggattaccgggaacagataatgtatgtgaagcaaaagagagagtttcttgctgtaataacaacaataagagagtactatcaactgatactgagaaggagattgaatcatcatcaaggaaaactgaaacatcccctcctcgaaaggctcagattgttggatggccaccagttagatcttacaggaagaacaacattcagagtaagaagaatgaatctgagcatgagggtcaaggaatctatgtgaaagtaagtatggatggtgcaccatacttgaggaaaatagatctgagttgttacaaaggatactcagagttgcttaaagctttagaagtgatgttcaaattctctgtgggagagtactttgagagagatggatataaaggttcagactttgtgcctacttatgaagacaaagatggtgattggatgctcattggtgatgttccatgggagatgttcatatgtacgtgcaagagactaaggatcatgaaaggatcagaagccaaaggtttaggctgtggtgtatga

SEQ ID NO: 152 - DNA, 3930 bp, Medicago truncatula
  misc_feature 1..3930: note = ORF of protein Skl
  source 1..3930: mol_type = unassigned DNA
atggaaacagaggcattgagttctgaacagacgaagagtaaaatggaggcagagacattgagtactaatccccctcctggttttcttattcgcgcacttccggctgttatacccgcgcttttgatttcgataggatatgttgaccctggaaagtgggtggcaagtatagaaggtggtgcgaggtttggttttgatctggtggcttttgcgctaattttcaattttgccgctatcttctgtcagtacttatccgcaagggttggtgtaatcactggacgagatcttgctcagatttgcagtgatgaatatgatacatggacatgccttctccttggaattcaagcagaactgtccgtgattatgctagatcttaacatgatcttgggcatggcacaaggattaaatcttatttttgggtgggacttgttcacctgtgtctttttaactgccactggtgctgtttttcatattcttcttgctatcctcttggacattgagaagacaaaattcctaggacagtttgtggcaggctttgtattactttcttttatacttggagtattcatccaatccgaagttccagtttccatgaatggaatactaataaatttaagcggggagagtacatttatgctgatgagtcttctcggagcaactcttgtgcctcacaacttttaccttcattcttctattgtacagtggcatcagggaccggcaaacatttctaaagatgcactgtgtcataaccattttttggccctcttatgtgtcttcagtggcctttatttggtaaattatattctgatgaccaccttagcaaatgagttctacagtacaggtcctgttttgcttatggaacaggtgttacacagcccaatagccctgattggctttgtactcattctgtttcttgccaatcaaactgcagcattaacttggagtttaggcggagaagttgtagttaatggtttcttaaaactagacataccaggttggcttcattatgcgacaattagagtgattgctgtgctgcccgccctttattgtgtctggagttcaggagctgaagggatctatcaactactaatattcactcaggttttagtagctttgcagcttccgtcgtctgtgattcctctttttcgagttgccttgtctagatcaataatgggtgcacacaaggtctcccagtcaatggagcttttggcgttgaccatattccttggggtgcttggcatgaatatcatgtttttgggagagatgatatttggcagtagtgattgggcgtgtgatttgaggtggaatttggggaatggcgtgtctgtcttgttttcagttctcctcattgctggttttttatcaatatgtttgatgcttcggttagctaccacacctttaagatctgccagcattcaattaaatgctcaggtcttaaactgggatatgccagaggctgtactaaatccgccggttgatggtgaagaatcacatgtaactgaaacagtgggtcatgaagatgcgtctttccaagctgacgaaccaaaaccagctttggcaaggtcattagagtaccctgaagtatcacttgctagttttcgtcctgatctacatctacctgaaactgtaatggaacctgatccccaggtgaatgctttaaaggaaaatcattctgttgcaccatcagtttccacatctgattcagggacagtatcaaaaactgtagctaatgatacttctagttctgattctaaacttaaagacacgaaaactatcattgaagcaaatgccccaattgagaagaccgtggaaattgaagatgattctaatgttgaaagggatgatgacgatgtagactcatgggaaactgaagaatcatccagagcagtcttggcaaatgccccgtcatcaacatcagaaggtcctccatcattcaggagtattagtgggaaaagtgatgacggtggttgtagctttggaagtctatcgagaatagaaggcttaggacgtgctgcaagacgtcagctagctgctactcttgatgaattttggggacagctttatgatttccatggtcaagcaacccaggcagcgaaggccaagaaaattgatgttttgctggggatgggagtagattcaaaacccaccgcttccttgcagaaaatggatgcatgtggaaaggactattctgaatacttggtatctgtagggggtagagcttccgacaatttaatcaacgctggcccgtatgactattccaatcagcctaggatgcaaagtaattcagagtctgcatatggtcttcaaaggagttcttcctcagtgcgggcaagtcccattcagttattggatgcatatgtacaaagttccaaccgcaatctcaatgattctggtgagaggcgctattccagtgttcgcaatcttcattcatctgaggcctgggattatcaaccagctacaattcatggttatcagaccgcatcctatcttagccggggtgtgaaagatagaagttctgaaaacataaatggttcaatgccattgacatcactgaagtccccttccacaggtaatccaaactatagggattcccttgcattcgttttgggaaaaaagttgcataatggttcaggtgtaggccatccccctgggtttgaaaatgtagctgtctctagaaatcgccaactacaaactgagaggtccaattatgattctagctcccctggagctgcagctaatacagtcagttcagttaacactaagaaataccacagcttgccggacatttcaggatactccattccccacagagctggctatgtgtctgataagaatgcaccgtgggatggttctgttggatatggttcttttgctggcaggatgggttatgaaccatctatgtattcaaattctggatcaagagctggaggagctcatttggcctttgatgaagtctctccatacagagaggcactctcttcgcagtttagttctggttttgacactggatccctctggtctagacagccttttgagcagtttggtgttgctggtaaaattcataatgttgcaatggaaggtgcgggaagcaggcctaatgcaattgttcaagaaattacatttgaggatatagaggggaaacttcttcagtctgttagacttaccattatgaagctcttaaaactagagggatctgattggttgtttaaacagaatgatggaattgatgaggatctgattgatcgtgtagcagcaagggataagtttgtttatgaaattgaagctagggaaacgaatcagggtattcatatgggtgacactcgttattttccttctgataggaaatctgtttcttcgatgaaagtcaacgaggcaaatgcctctagtctttcggtttcctcagttcccaactgcggggagggatgtgtatggagagcagatttaataataagctttggggtgtggtgtatccaccgtattcttgacctgtcacttttggaaagccggccagagctatggggaaaatacacctacgtgctcaatcgcctccagggcatcatcgaaccagctttttcgaagccccgtactccttcggccccttgtttttgcattcaagttccaaccacgcaccagcaaaagtcaagcccccctctttctaacgggatgctacccccaacggtaaaaccaggccggggaaaatacactactgcatcatcgttgcttgagctgattaaagatgtggagatcgctatctctagcaggaaaggacgtacaggtactgcagctggtgaggtggctttcccaaaggggaaggaaaatttggcatccgttcttaaacggtacaagcgaaggttatcatccaacaagcttgttggcaatcaggaggggactagttctcgcaagatcccgtcatcaggaccatacaaccaatag

SEQ ID NO: 153 - DNA, 3144 bp, Arabidopsis thaliana
  misc_feature 1..3144: note = ORF of protein Sps1
  source 1..3144: mol_type = unassigned DNA
atggtgggaaacgactgggtgaatagttacctggaggcgattctcgcagccgaacctggaatcgccaattccaaaccgcctggaaccggcgattccaaatcgtctcttctgctgagagagcgtggccatttcagtcccactcgttatttcgttgaagaggttatcacaggctttgatgagactgatcttcaccgttcctgggttcaggctgctgcaaccaggagtccgcaagagcgtaacacaaggttggagaatttgtgctggaggatctggaatttggctcgccagaagaagcaggttgaagggaagaatgctaagcgtgaagctaaaagggagcgtgagcgtgagaaagcaaggagggaagtaactgctgaaatgtctgaagatttctctgaaggagagaaggcagatttgcctggtgaaatcccaactcctagtgataacaacaccaaaggaagaatgtctcggattagttctgttgatgtctttgagaattggtttgctcaacacaaagagaaaaagctttacatcgtgttaataagtcttcatggtttgatacgtggtgaaaacatggagcttggtcgcgattctgatactggtggccaggtgaagtatgttgtggaacttgcaagggcattaggctcaatgccaggagtctatcgtgttgatttgttgaccagacaggtcaccgcgccagatgtggattccagctactctgaaccgtctgagatgttaaatcctatagacacagatatagagcaggagaatggagagagtagtggcgcttatataatccgtattccttttggcccgaaagataaatatgtaccaaaagagctcctctggcctcatatccctgaatttgttgatagggcacttagccatattatgcagatttcaaaggttcttggtgagcaaattggtggtggacaacaagtatggccagtttctattcacgggcattatgcagatgctggtgattccactgctcttttatctggggctctgaatgtaccaatggtttttactgggcattctcttggtcgtgataagcttgagcaactcctgaaacaaggacggccaaaagaagaaatcaattccaactacaagatatggagacggatagaggcagaggagttatgtcttgatgcttctgagattgtcataactagtacaaggcaagaggtagacgagcaatggcgcctttatgatggttttgatccggttttggagcgaaaactcagagcaagaatgaaaaggggtgtgagttgtctaggcagatttatgcctcgcatggttgttattcctccaggaatggaatttcatcacattgtaccacatgatgtggatgcagatggagatgatgaaaatccacaaactgctgatccaccaatttggtctgagattatgcgcttcttctctaacccgcgcaagcctatgattctcgctcttgctcggccagaccctaaaaagaacttggttactctagtcaaagcctttggagaatgccgtccattacgggagcttgcgaaccttacattgataatgggaaatcgaaatgatattgatgagttgtcctccacgaattcctctgtgctgctctcaattctgaagctaatagacaagtacgacttatacggtcaagtggcaatgcctaagcatcaccaacaatctgatgtgcctgaaatttaccgtttggcagccaaaacaaagggagtatttataaatccagcgttcatcgaaccatttggactgactctaatcgaggcaggagctcatgggctccccactgttgcaacaattaatggaggacctgtcgatatacatcgggttcttgacaatggtcttctagttgatcctcatgatcagcaagctatagctgatgcgctcctgaaactagtttctgacagacagctatggggaagatgcagacaaaatggcctaaataacattcacctgttttcatggccagagcactgcaaaacatatttggctcggatagcttcgtgcaagcaaagacatcctaaatggcagagagttgagtttgagaactcagactccgattcacccagcgactctcttagagatataaatgacatctccttgaatctaaaactttccttagatggcgaaaagagtggaagtaacaatggtgtagatactaacttagatgctgaagacagagctgctgaacgtaaagctgaggtagagaaagctgtttcaaccttggcacaaaagagcaaaccaacagagaaatttgattccaagatgcctactttgaaaagacggaaaaatatatttgtcatttctgtggactgtagcgcaacctcagatcttcttgcagtggtgaaaacagttattgatgctgcaggaagaggcagttcaacaggattcattctctcaacctcaatgacaatatctgagactcacacagcactgctttcaggagggttgaaaccacaagattttgatgctgttatttgcagcagtggaagtgaactctacttcacgtcctctggttcagaggataagaccgcacttccttatacactcgatgcagattaccattcccatatagaattccgctggggtggagagagcttgaggaagactttgatccgttggattagttctgttgaagagaagaaaaagacgaaaaaaggagagattttagttgaagacgagtcttcttctaccaactattgcctatccttcaaagtgaaagatcccgctttgatgcctccaatgaaagagctgaggaaattgatgagaaatcaagcattacgttgcaatgcagtttactgccaaaatggagctaggcttaatgtaatccctgttcttgcatcacgatctcaggcccttaggtatttgctagtgcgatggggcattgacttatcgaacatggtggtgttcgtgggagattcaggtgacacggattacgaaggcttgcttggaggaattcacaaaacagtgatacttaaaggcttagccagtgacttgcgtgagcaacccggcaacagaagctacccaatggaggatgtgactccactgaacagtcccaacatcaccgaggctaaagaatgtggtcgtgacgccattaaggtcgctctagagaagcttgggatcagtctcctcaagccttga

SEQ ID NO: 154 - DNA, 1122 bp, Arabidopsis thaliana
  misc_feature 1..1122: note = ORF of protein Spt
  source 1..1122: mol_type = unassigned DNA
atgatatcacagagagaagaaagagaagagaagaagcagagagtgatgggagataagaaattgatttcatcttcttcttcttcctcggtttacgatactcgtatcaatcatcatcttcatcatcctccgtcttcttccgacgaaatctctcagtttctccggcatattttcgaccgttcttctcctttaccttcttactactccccggcgacgactacaacgacggcgtctttgattggtgtgcacgggagcggtgacccacatgcagataactcgagaagtctcgtttctcatcatccaccgtcagattctgtgcttatgtcgaaacgtgtcggagatttctctgaggttttaatcggcggaggatcaggctcagccgccgcgtgttttggtttctccggtggtggtaataataacaacgttcaaggaaatagctctgggactcgagtatcgtcttcttccgttggagctagtggcaacgagacagatgagtatgactgtgaaagcgaggaaggaggagaagctgtagttgatgaagctccctcttccaagtcaggtccttcttctcgtagttcatctaaaagatgcagagctgctgaagttcataatctctctgagaagaggaggagaagtagaattaatgaaaaaatgaaagctttacaaagtctcatccctaattcaaataagacggataaggcttcaatgcttgatgaagccattgagtatctgaaacagcttcagctccaagttcagatgttgactatgagaaatggaataaacttgcatcctttgtgtttacctggaactacattacacccattgcaactctctcagattcgaccccctgaagcaaccaatgatcctctgcttaatcataccaatcagtttgcttcgacttctaatgcaccggaaatgatcaatactgtggcttcttcatacgctttggaaccttctattcgcagtcactttggacctttccctctccttacttcacccgtggagatgagtcgggaaggtgggttaactcatccaaggttgaacattggtcattccaacgcaaacataaccggggaacaagctctgtttgatggacaacctgacctaaaagatcgaattacttga

SEQ ID NO: 155 - DNA, 1488 bp, Arabidopsis thaliana
  misc_feature 1..1488: note = ORF of protein Stn8
  source 1..1488: mol_type = unassigned DNA
atggcctctcttctctctcccgccacccccaccgccacctccgccgcatttcattcctgctccaccgccggattctccactcccactcacatttcatcccagaacagcagcctgtccctcctcagccgccggggttgtatgatgagatgcagtttttctccgcaggacattcctgttgattccctctctcatctccctcctttccttgactttcagaactccctcgccacattttcggatacccagaaatgggggttctttgtctccgccggaatcgtctggttttacctcacggcgaggcctggtgttctcatcggcgccattgacgcctaccttctcgctcccctccagcttggcctcgacactctgattggaaggaggttgaagaggtccgatttcttggtcactgagaaactcggcgaaggctctttcggcgtcgtctatgccggcgtcctactccccaagaattctactcttgttgatgacgttagggttagcaaagcaagagctaaagcgatggactttaccggcgaattcaagcagagagtcatcctcaagaaggtcaaggtgggagtacgaggggccgaggaatttggcgagtacgaagagtggttcaactaccgactctcaagggccgctcctgacacctgcgctgagtttctcggaagctttgtcgccgacaagaccaatactatgtttactaaaggcggcaaatggcttgtctggaggttcgagggagaccgtgatctcgctgattacatgaaagatcggagctttccttctaatttggagtccatcatgtttgggcgcgttctccaaggagtcgagtctgtgaaacgtcgtgcactgatcattaagcagattatgcgccagatcattacatctctcaggaaaatccatggcacgggcattgttcaccgggacgtgaagcctgccaacttggtggttacaaagaagggccagattaagctcatcgactttggtgccgctgctgatcttcgcattggcaagaattacatcccggaacgcactttactcgatcctgactactgtcctcctgagctctatgtcctcccggaggaaacgccaagccctcctccggagcctatagccgctttgctttccccgattctctggcagttgaacagtccggatctgtttgatatgtattctgctgggatagtgcttctccaaatggcggttccgaccttaagatctactgccggtctgaagaattttaatctggagatcaagtctgttgaatatgatcttaataggtggcgggaaaggactcggacgaggccagacttgagcatactagatctggactcgggcagagggtgggacctcgtcactaaactcatatccgagagaggttctctgagacgtggacgcctttcagcagctgctgctctcagacacccttattttttgttgggcggtgatcaagcggccgctgttctttcaaagctcagtttcagcaagtga

SEQ ID NO: 156 - DNA, 1218 bp, Arabidopsis thaliana
  misc_feature 1..1218: note = ORF of protein Tap46
  source 1..1218: mol_type = unassigned DNA
atgggtggtttggctatggaggaaatgcctttatcggtgctattcgagcaagctaggaaaattcatcttgctgcttcagagtctggtgttgatcaggatgttgtgaagaaaggatgtgagatgtttcagaagtgcgaagatatgatcgggaagctggcgctgttttcgtctaacgagactaaagaagatattagcaccaacaatctcaaatatcttttggtgccttattatcttgcggagttgacagagaaaatcatacaggaggaccggattcagattgtcaaagcctcgtatgctaagttgaaggagttcttttcgttctgtgaggcaatggagcttgttccggatgaggagttggaggcttcttcacgaggaggttctggtgcacctgctgatcgaagggctctcaagatagctcggttcaaacgtcaaaaggctgcagaggcaaagcttcttgaaattaaagagaggaaggagcgacgtggacgttccactaaagcatcagctttgtcgactccggttgaatctggagaagatgatattccagatgatgacagcgaagaagagagagaggcctggctctcctcaattaacttggctatttgtaaggctattgatctgttggaaatgctaaagagagaagaggaaatgctctctgcaataaaggaaagacagttgaaggatggagagggtggattttctcgggatgctcttgatgatcgtacaaagaaagctgaaacctggcacagagacgctgctgcaaggatacagtactctaaaccggcacaaccaatcacttgtgccacatttgcacaagatgtgttagaaggaagagcttcggtatcacaaggtcacgaacacaagaaccaacctcttatattcggtccagcaagcatcgtgggtggaccgctctctaccgagagagaaagaatgatagctcaggtcttccaaccaagtcacaggatgccaacaatgtgcatagaggatgctgggttaacagagatgaacataatgaatgattggcaagagcagacgaaaaaagccattgaagaagcaaccacctcatggtacaatgataaacctctaagaagaaaagaagaagacgaggaagatgatgatgaagacgaagaagctgtgatgaaagctagagctttcgatgattggaaggatgataatcctcgcggtgcaggtaacaagaaactcacaccttgtggctga

SEQ ID NO: 157 - DNA, 972 bp, Arabidopsis thaliana
  misc_feature 1..972: note = ORF of protein Topp6
  source 1..972: mol_type = unassigned DNA
atggatcctggtactttaaactcggtgatcaataggttgcttgaagctagagaaaaaccaggaaagattgttcagttgtctgaaacagagatcaaacagctctgtttcgtctctagagatatcttcttgagacaaccaaatctcttggaacttgaagctcctgttaaaatatgtggggacattcatggacaatatccggatctcttgagactattcgaacatggcggataccctcctaattcaaactacttgtttcttggagattatgtcgatcgcggcaagcaaagcctcgaaacgatttgtcttttacttgcttacaagattaagttccctgaaaacttcttccttctcagaggaaaccatgaaagtgcatcaatcaatcgtatttacggcttctatgacgagtgtaaacgtagattcagtgtcaagatttggcgaatcttcactgattgcttcaactgtctccccgtcgctgcactcatcgatgagcggattttttgtatgcatggtgggctctccccggagctgctaagcttgaggcagattagggatattcgtcgtccaacggatattcctgatcgtggtttactctgtgatctcttgtggtctgatcctgataaagatgttagaggttgggggcctaacgatcgcggagtttcttacacttttggatcagatatagtttctggatttcttaaaagactcgatcttgacctcatttgtagggctcaccaggttgttgaagatggattcgagttctttgcgaataagcagctcgtaacgatattctctgcgccgaattactgtggggaatttgacaatgcaggtgcgatgatgagtgtgtctgaggatttgacctgctcttttcagatcttaaaatctaatgacaagaaatcaaagttcagtttcggaagcagaggtggtgctaaaactagcttcccttatcctaaagtgaaggattgtaattggtag

SEQ ID NO: 158 - DNA, 1350 bp, Arabidopsis thaliana
  misc_feature 1..1350: note = ORF of protein TubB6
  source 1..1350: mol_type = unassigned DNA
atgagagaaatccttcacattcaaggtggtcaatgtgggaaccagattggttccaagttctgggaagttgtatgtgatgagcatggtattgatcccactggtcgttacgttggaaactctgatctgcagttggagcgtgtcaatgtttactataacgaggcatcctgcggaagatatgttccccgtgcaattctcatggatcttgagcctggtactatggacagtgtcagaactggaccttatggtcaaatcttcaggcctgacaactttgttttcgggcaatctggtgctggaaacaactgggctaaagggcattacactgaaggagctgagcttattgatgctgtactcgatgttgtacgcaaagaggctgagaattgcgactgtcttcaaggtttccaagtatgtcactcacttggtggaggcaccgggtctggaatgggaactctgctcatatctaagatcagggaagagtatcctgatagaatgatgcttacattttctgtcttcccatcaccaaaggtctctgacacagtggttgagccatacaatgcgacactttcagttcatcagctggtggaaaatgctgatgagtgtatggttttggacaatgaagccctttacgacatctgttttagaacacttaagcttaccactcctagctttggtgatctgaatcatcttatctctgcaaccatgagtggagttacatgctgtcttaggttcccgggtcagctcaactctgatctgaggaagctcgcagtgaacctcattcctttccctcgtctccactttttcatggttggttttgcccctctcacctcccgtgggtctcagcagtaccgtgcactcactgtccctgagctgacccaacagatgtgggattcaaagaacatgatgtgcgcagcagacccgcgccacgggcggtacctaactgcctcggccatgttccgtggcaagatgagcacaaaagaagtggacgagcagatgataaacgtacagaacaaaaactcttcctactttgtggaatggattccaaacaacgtgaagtcaagcgtctgtgacatagctccccgaggtctctcaatggcatcgacatttattgggaattccacatcgatccaagagatgtttaggcgggtgagcgagcagttcactgctatgttcaggaggaaagctttcttgcattggtacacaggtgaaggaatggacgagatggagtttactgaagctgagagcaacatgaacgatctagtctcagagtaccagcaataccaagacgcaactgcagatgacgaaggcgagtatgaagaagacgaggatgaagaagagatattggatcatgagtga

SEQ ID NO: 159 - DNA, 2700 bp, Arabidopsis thaliana
  misc_feature 1..2700: note = ORF of protein TubB8
  source 1..2700: mol_type = unassigned DNA
atgcgagagattcttcacatacaaggtggccaatgcggaaaccagatcggagctaagttctgggaagtagtttgcgccgagcacgggatcgattcgacgggaagataccaaggagagaacgatttgcaactcgaacgagtcaatgtctactacaacgaagcgagttgcggaagattcgtcccacgcgccgtcctcatggatctagagcctggtactatggatagtgtcagatccggtccgtacggtcagatcttccgtcctgataacttcgtatttggtcaatccggtgccggtaacaattgggctaaaggacactacactgaaggtgctgagcttatcgattccgttctcgatgttgttcgtaaggaagctgagaattgtgactgcttgcaaggattccaagtgtgtcactcattaggaggaggaactggatctggaatgggaacgttgttgatttcaaagattagggaagagtatccagataggatgatgctgacgttttctgtattcccatcaccgaaagtgtcagacactgttgttgagccttataatgcaactctctctgttcatcagcttgtggagaatgcagatgagtgtatggttcttgacaatgaagctctctatgacatttgcttcagaactcttaagctcaccactccaagctttggagacttgaatcacctcatctctgcaaccatgagtggtgtcacttgctgccttcgtttcccgggtcagctaaactctgacctgcgcaagcttgcagtgaatttaatcccattccctcgtctccacttcttcatggttgggtttgcaccactcacatctcgtggatcacagcaatacagagccttaaccgtccctgagctcactcaacagatgtgggatgccaagaatatgatgtgtgcagctgatccacgccacggcaggtacttgacagcctcagccatgttcagaggcaaaatgagcacgaaagaagttgacgaacagatgatcaacgttcagaacaagaactcgtcatactttgtggaatggatcccaaacaacgtgaaatcaaccgtgtgtgacattccaccaacaggtctgaaaatggcttcgactttcatcgggaactcgacatcgatccaggagatgtttaggcgtgtgagcgaacagttcacagcgatgttcagaagaaaagctttcttgcattggtacacaggtgaaggaatggacgagatggagttcacagaggcagagagcaacatgaatgatttggtatcagagtatcaacagtaccaagacgcaacggctgatgaggaagaagggtatgagtatgaagaagacgaagtggaagtgcaggaggagcaataaatgcgagagattcttcacatacaaggtggccaatgcggaaaccagatcggagctaagttctgggaagtagtttgcgccgagcacgggatcgattcgacgggaagataccaaggagagaacgatttgcaactcgaacgagtcaatgtctactacaacgaagcgagttgcggaagattcgtcccacgcgccgtcctcatggatctagagcctggtactatggatagtgtcagatccggtccgtacggtcagatcttccgtcctgataacttcgtatttggtcaatccggtgccggtaacaattgggctaaaggacactacactgaaggtgctgagcttatcgattccgttctcgatgttgttcgtaaggaagctgagaattgtgactgcttgcaaggattccaagtgtgtcactcattaggaggaggaactggatctggaatgggaacgttgttgatttcaaagattagggaagagtatccagataggatgatgctgacgttttctgtattcccatcaccgaaagtgtcagacactgttgttgagccttataatgcaactctctctgttcatcagcttgtggagaatgcagatgagtgtatggttcttgacaatgaagctctctatgacatttgcttcagaactcttaagctcaccactccaagctttggagacttgaatcacctcatctctgcaaccatgagtggtgtcacttgctgccttcgtttcccgggtcagctaaactctgacctgcgcaagcttgcagtgaatttaatcccattccctcgtctccacttcttcatggttgggtttgcaccactcacatctcgtggatcacagcaatacagagccttaaccgtccctgagctcactcaacagatgtgggatgccaagaatatgatgtgtgcagctgatccacgccacggcaggtacttgacagcctcagccatgttcagaggcaaaatgagcacgaaagaagttgacgaacagatgatcaacgttcagaacaagaactcgtcatactttgtggaatggatcccaaacaacgtgaaatcaaccgtgtgtgacattccaccaacaggtctgaaaatggcttcgactttcatcgggaactcgacatcgatccaggagatgtttaggcgtgtgagcgaacagttcacagcgatgttcagaagaaaagctttcttgcattggtacacaggtgaaggaatggacgagatggagttcacagaggcagagagcaacatgaatgatttggtatcagagtatcaacagtaccaagacgcaacggctgatgaggaagaagggtatgagtatgaagaagacgaagtggaagtgcaggaggagcaataa

SEQ ID NO: 160 - DNA, 1044 bp, Arabidopsis thaliana
  misc_feature 1..1044: note = ORF of protein Uba1a
  source 1..1044: mol_type = unassigned DNA
atggcgaaaaccctagacaaatctaagaagcgcaaactcgtgaaatccaaatccacgaatttcgataagaaacagaagattaacaaacaacaacaacaaccagaatcctcaacgccatattcaagttcaagctccagctctgattctagcgattccgaatcagacaacgaattcgacccagaagagctccgtgagcttctacaaccttattccaaggaccagcttgtcgatctagtttgttccgcttctcgaatcggatcttcgatctattccgccgtcgtagaagccgcagatcgtgatgttacacacaggaaaatcttcgtttatggtcttccatgggagacgactcgcgagactcttgttggagtctttgaaggttatggtgagattgaagagtgtaccgttgttatagataaggctactggtaaggctaaagggtttggatttgttatgttcaagactaggaaaggagctaaagaagctcttaaagaaccgaagaagaggattcttaatagaactgccacttgccagcttgcttctatgggtcctgcggcttcaggaaaggggcatgatcaacctggtccggtgaagattagtatgggttcgatggctaatcatggacagcctcagcaacagcaggtacaaggtcaacatgtgtttaacggtggtggaatggctgcttcgccgtttatgttagggaaccaataccatccgctttatggagctgggatgttgggaaatccagctttagcagcagcagcagcagctgggggaggttacatgtatccgatgttagcgggtgctctagcacatggcgggttgggttctgatatggtacagtctagtcaaatgggtgggattggtgttgatccaagcgttggtgctgctggtctttcagctctaggttcttactttagaggtcagagtctgcctagtacttatcctgattcggatgctggtggaaaaagagggacgggtaaagattcggatgctggtggctcctcctttcatggctactcaaactattcatggtacgttcactga

SEQ ID NO: 161 - DNA, 1854 bp, Arabidopsis thaliana
  misc_feature 1..1854: note = ORF of protein Vim3
  source 1..1854: mol_type = unassigned DNA
atggcgattgaaactcagcttccttgcgacggtgacggtgtgtgtatgcggtgtcaggtgaatcctccgtcagaagagactctcacttgtggcacgtgcgtcactccatggcacgtgccgtgtctcctccccgaatcactcgcttcttccactggagagtgggagtgtcccgattgctccggcgttgtcgttccctccgccgctccgggtaccggaaacgctcgacctgaatcttccggttcagttctcgttgctgcgatccgtgcgattcaggctgatgagactttaaccgaagctgagaaagccaaaaaaaggcagaaactgatgagtgggggtggtgacgatggtgtcgatgaagaagagaagaagaagttagaaatcttttgttctatttgcattcaattgccagaaagacctatcacgacaccgtgtgggcacaatttctgtttgaaatgtttcgagaaatgggcagtaggtcaagggaagctaacttgtatgatatgccgaagcaaaattccgagacatgtggcaaaaaatcctcgcatcaacttagctctagtttctgctattcgtttagcaaatgttaccaaatgttctgttgaggcaactgcagccaaggttcatcatattatccgcaaccaagaccgtcctgagaaagcatttactaccgagcgggcagtaaaaactgggaaagctaatgctgctagcggtaagttttttgtgacaatacctcgtgatcattttggtcccataccagctgagaatgatgtcactagaaagcaaggtgttttggttggagaatcttgggaggacaggcaagagtgtaggcagtggggagctcatttcccgcatattgctggcattgccgggcaatcagcggttggagctcagtctgtggccctctctggaggttatgacgatgatgaggatcatggtgaatggtttctctacacaggaagtggtggaagggatctcagtggaaacaaaagaattaacaagaaacagtcgtctgaccaggcgtttaaaaacatgaatgaatctctaagacttagttgcaaaatgggctatcctgtccgagttgtcaggtcttggaaggagaagcgttctgcatatgcccctgctgaaggtgtgagatatgatggggtctatcgaattgagaagtgctggagtaatgttggagtacagggttcttttaaggtctgtcgttacctgtttgttagatgtgacaatgagccagctccatggaccagtgatgagcatggcgatcgtccaagaccgttgcctaatgttccggagcttgagactgctgctgacctgtttgtgagaaaggagagtccatcatgggatttcgatgaagctgagggtcgttggaaatggatgaagtctcctcctgttagcagaatggctttggatcctgaggagaggaagaagaataagagagcaaaaaatactatgaaggccagacttctgaaagaatttagttgccaaatctgtcgggaagtgctgagtcttccagtgacgacgccttgtgcacacaacttctgcaaagcatgcttagaagcgaagtttgctgggataactcaactgagagagagaagcaatggcggacgtaaactacgtgcaaagaagaacatcatgacctgcccttgctgcacgacggatctctccgagtttctccaaaacccgcaggtgaacagagagatgatggagataatagagaattttaagaagagtgaggaagaggctgatgcatccatttctgaagaagaagaagaagaatccgaacctccaactaagaagattaagatggataacaactctgttggtggtagtggtacaagtctctcagcttaa

SEQ ID NO: 162 - protein, 10 aa, synthetic construct
  REGION 1..10: note = cPEPcpk3
RSSKTELWTR

SEQ ID NO: 163 - protein, 12 aa, synthetic construct
  REGION 1..12: note = cPEPdcl1
SRAFGSVFFSGD

SEQ ID NO: 164 - protein, 10 aa, synthetic construct
  REGION 1..10: note = cPEPnsp1_3
DYGTKSNIRS

SEQ ID NO: 165 - protein, 10 aa, synthetic construct
  REGION 1..10: note = cPEPnsp1_1
EPNPTSDHIL

SEQ ID NO: 166 - protein, 10 aa, synthetic construct
  REGION 1..10: note = cPEPnsp1_2
LKVLSLSFHH

SEQ ID NO: 167 - protein, 10 aa, synthetic construct
  REGION 1..10: note = cPEPnsp1_4
PKHGDPIFNR

SEQ ID NO: 168 - protein, 10 aa, synthetic construct
  REGION 1..10: note = cPEPnsp1_5
YKCGTMVEEP

SEQ ID NO: 169 - protein, 5 aa, synthetic construct
  REGION 1..5: note = cPEPnsp1 5aa
LSFHH

SEQ ID NO: 170 - protein, 20 aa, synthetic construct
  REGION 1..20: note = cPEPnsp1 20aa
LKVLSLSFHHFWMIHITMVT

SEQ ID NO: 171 - protein, 30 aa, synthetic construct
  REGION 1..30: note = cPEPnsp1 30aa
FLIGLKVLSLSFHHFWMIHITMVTSMSMRY

SEQ ID NO: 172 - protein, 40 aa, synthetic construct
  REGION 1..40: note = cPEPnsp1 40aa
LWNQIQHQITFLIGLKVLSLSFHHFWMIHITMVTSMSMRY

SEQ ID NO: 173 - protein, 60 aa, synthetic construct
  REGION 1..60: note = cPEPnsp1 60aa
MNESDNGDAALEGTVVRKSGGNKKGAAKANGSNSNNGNNKDGRWAEQLLNPCAVAITGGN

SEQ ID NO: 174 - protein, 80 aa, synthetic construct
  REGION 1..80: note = cPEPnsp1 80aa
MNESDNGDAALEGTVVRKSGGNKKGAAKANGSNSNNGNNKDGRWAEQLLNPCAVAITGGNLNRVQHLLYVLHELASTTGD

SEQ ID NO: 175 - protein, 10 aa, synthetic construct
  REGION 1..10: note = cPEPgapc
DKDKAAAHLK

SEQ ID NO: 176 - protein, 10 aa, synthetic construct
  REGION 1..10: note = cPEPbri1
HQRSHGRRPA

SEQ ID NO: 177 - protein, 10 aa, synthetic construct
  REGION 1..10: note = cPEPbak1
KRLKEERTQG

SEQ ID NO: 178 - protein, 10 aa, synthetic construct
  REGION 1..10: note = cPEPshy2
EENRSELLQR

SEQ ID NO: 179 - protein, 10 aa, synthetic construct
  REGION 1..10: note = cPEPpizza
PRFQNHDKKK

SEQ ID NO: 180 - protein, 10 aa, synthetic construct
  REGION 1..10: note = cPEPmrb1
NSEITKTAND

SEQ ID NO: 181 - protein, 10 aa, synthetic construct
  REGION 1..10: note = cPEPtap46
NKGKTVEGWR

SEQ ID NO: 182 - protein, 10 aa, synthetic construct
  REGION 1..10: note = cPEPspt
ASGNETDEYD

SEQ ID NO: 183 - protein, 10 aa, synthetic construct
  REGION 1..10: note = cPEPga2ox7
SDFSSKYRED

SEQ ID NO: 184 - protein, 10 aa, synthetic construct
  REGION 1..10: note = cPEPphyb
LRETDLKDRR

SEQ ID NO: 185 - protein, 10 aa, synthetic construct
  REGION 1..10: note = cPEPhag5
VERRETPSGE

SEQ ID NO: 186 - protein, 10 aa, synthetic construct
  REGION 1..10: note = cPEPshr
HGEARDSEEV

SEQ ID NO: 187 - protein, 10 aa, synthetic construct
  REGION 1..10: note = cPEPmapkkk3
KEPEQRKRKS

SEQ ID NO: 188 - protein, 10 aa, synthetic construct
  REGION 1..10: note = cPEPmapkkk5_1
PLTGGRRDSK

SEQ ID NO: 189 - protein, 10 aa, synthetic construct
  REGION 1..10: note = cPEPmapkkk5_2
HRDIKGANLL

SEQ ID NO: 190 - protein, 10 aa, synthetic construct
  REGION 1..10: note = cPEPring1
KERTTDSTKP

SEQ ID NO: 191 - protein, 10 aa, synthetic construct
  REGION 1..10: note = cPEPros1
KLQREDKDQL

SEQ ID NO: 192 - protein, 10 aa, synthetic construct
  REGION 1..10: note = cPEPjar1
PETNFERDSS

SEQ ID NO: 193 - protein, 10 aa, synthetic construct
  REGION 1..10: note = cPEPcoi1
KHRHISEKPL

SEQ ID NO: 194 - protein, 10 aa, synthetic construct
  REGION 1..10: note = cPEPabcg34
ERKFRLHKSP

SEQ ID NO: 195 - protein, 10 aa, synthetic construct
  REGION 1..10: note = cPEPagb1
IRLDTGEEPD

SEQ ID NO: 196 - protein, 10 aa, synthetic construct
  REGION 1..10: note = cPEPwus
RWTPTTEQIK

SEQ ID NO: 197 - protein, 10 aa, synthetic construct
  REGION 1..10: note = AhEIN2
VLKRYKRRLS

SEQ ID NO: 198 - protein, 10 aa, synthetic construct
  REGION 1..10: note = AhBRI1
RHRNLVRLQG

SEQ ID NO: 199 - protein, 9 aa, synthetic construct
  REGION 1..9: note = AhBAK1
ARGLAYLHE

SEQ ID NO: 200 - protein, 13 aa, synthetic construct
  REGION 1..13: note = AtEIN2cPEP1
MGCKYRRRCSFRV

SEQ ID NO: 201 - protein, 23 aa, synthetic construct
  REGION 1..23: note = AtEIN2cPEP2
MTWWQLLCFSILPPSYANMLQLA

SEQ ID NO: 202 - protein, 13 aa, synthetic construct
  REGION 1..13: note = AtEIN2cPEP3
MPICCSSHKRCDW

SEQ ID NO: 203 - protein, 7 aa, synthetic construct
  REGION 1..7: note = AtEIN2cPEP13
MICQRTF

SEQ ID NO: 204 - protein, 10 aa, synthetic construct
  REGION 1..10: note = cPEPein2_1
NPVERSRHRD

SEQ ID NO: 205 - protein, 10 aa, synthetic construct
  REGION 1..10: note = cPEPein2_2
VLKRYKRRLS

SEQ ID NO: 206 - protein, 10 aa, synthetic construct
  REGION 1..10: note = cPEPein2_3
KQIRLEKTLA

SEQ ID NO: 207 - protein, 10 aa, synthetic construct
  REGION 1..10: note = cPEPgfp/_1
ELDGDVNGHK

SEQ ID NO: 208 - protein, 10 aa, synthetic construct
  REGION 1..10: note = cPEPgfp_2
DLTSKGEELF

SEQ ID NO: 209 - protein, 10 aa, synthetic construct
  REGION 1..10: note = cPEPgfp_3
DKQKNGIKAN

SEQ ID NO: 210 - protein, 10 aa, synthetic construct
  REGION 1..10: note = cPEPgfp_4
ERVKVMQHTE

SEQ ID NO: 211 - protein, 10 aa, synthetic construct
  REGION 1..10: note = cPEPgfp_5
RAGEDHLLQG

SEQ ID NO: 212 - protein, 10 aa, synthetic construct
  REGION 1..10: note = cPEPgus
ADGARPRING

SEQ ID NO: 213 - protein, 10 aa, synthetic construct
  REGION 1..10: note = cPEPluc
KRYGLNTNHR

SEQ ID NO: 214 - protein, 11 aa, synthetic construct
  REGION 1..11: note = cPEPartificial
RTFNSKVSPKN

SEQ ID NO: 215 - protein, 10 aa, synthetic construct
  REGION 1..10: note = scrambled cPEPnsp1
SITDKGYSRN

SEQ ID NO: 216 - protein, 12 aa, synthetic construct
  REGION 1..12: note = scrambled cPEPdcl1
FSGRDFSVAFGS

SEQ ID NO: 217 - protein, 10 aa, synthetic construct
  REGION 1..10: note = scrambled cPEPcpk3
KSWTSRTLRE

SEQ ID NO: 218 - protein, 10 aa, synthetic construct
  REGION 1..10: note = scrambled cPEPgfp
DGHLEGVKND

SEQ ID NO: 219 - protein, 10 aa, synthetic construct
  REGION 1..10: note = scrambled cPEPgus
RNADAGRGIP

SEQ ID NO: 220 - protein, 10 aa, synthetic construct
  REGION 1..10: note = scrambled cPEPluc
RNYKNGRTHL

SEQ ID NO: 221 - protein, 10 aa, synthetic construct
  REGION 1..10: note = Irrelevant Peptide
RNADAGRGIP

SEQ ID NO: 222 - protein, 19 aa, synthetic construct
  REGION 1..19: note = cPEPcpk3-HA
RSSKTELWTRYPYDVPDYA

SEQ ID NO: 223 - protein, 19 aa, synthetic construct
  REGION 1..19: note = scrambled cPEPcpk3-HA
KSWTSRTLREYPYDVPDYA

SEQ ID NO: 224 - protein, 30 aa, synthetic construct
  REGION 1..30: note = AtDCL1alt18
MLMLNPPRHHGILQRPTCLSLLLTIRLWNP

SEQ ID NO: 225 - protein, 30 aa, synthetic construct
  REGION 1..30: note = CrDCL1alt18
MLMLNPPRHHGILQRPTCLSLFLTIRLWNL

SEQ ID NO: 226 - protein, 30 aa, synthetic construct
  REGION 1..30: note = EsDCL1alt18
MLMWNPPRHHGTLQRPTCLSLLLTIRLQNP

SEQ ID NO: 227 - protein, 30 aa, synthetic construct
  REGION 1..30: note = AlDCL1alt19
MLMLNPLRHHGILQRPTCLSLFLTIRQLNP

SEQ ID NO: 228 - protein, 28 aa, synthetic construct
  REGION 1..28: note = CsDCL1alt19
MLNPQRHHGILQRPTCLFLFLTIRLWNP

SEQ ID NO: 229 - protein, 21 aa, synthetic construct
  REGION 1..21: note = RsDCL1alt15
MLMSNPPRHHGILQRPTCLSP

SEQ ID NO: 230 - protein, 21 aa, synthetic construct
  REGION 1..21: note = BnDCL1alt14
MLTSNPPQHHGIPQRPTCLSP

SEQ ID NO: 231 - protein, 21 aa, synthetic construct
  REGION 1..21: note = BoDCL1alt15
MLTSNPPQHHGIPQRPTCSSP

SEQ ID NO: 232 - protein, 21 aa, synthetic construct
  REGION 1..21: note = BRDCL1alt17
MLMSSPPQHHGIPQRPTCSSP

SEQ ID NO: 233 - protein, 13 aa, synthetic construct
  REGION 1..13: note = BsEIN2alt1
MGCKYRRRCSFWA

SEQ ID NO: 234 - protein, 13 aa, synthetic construct
  REGION 1..13: note = CgEIN2alt1
MGCKYRRRCSFWV

SEQ ID NO: 235 - protein, 13 aa, synthetic construct
  REGION 1..13: note = CrEIN2alt1
MGCKYRRRCSFWV

SEQ ID NO: 236 - protein, 13 aa, synthetic construct
  REGION 1..13: note = EsEIN2alt1
MGCKYRRRCPFWA

SEQ ID NO: 237 - protein, 13 aa, synthetic construct
  REGION 1..13: note = ThEIN2alt1
MGCKYRRRCPFWA

SEQ ID NO: 238 - protein, 13 aa, synthetic construct
  REGION 1..13: note = BrEIN2alt1
MGCEYRRRCPFWV

SEQ ID NO: 239 - protein, 13 aa, synthetic construct
  REGION 1..13: note = BoEIN2alt1
MGCEYRRRCPFWV

SEQ ID NO: 240 - protein, 13 aa, synthetic construct
  REGION 1..13: note = BnEIN2alt1
MGCEYRRRCPFWV

SEQ ID NO: 241 - protein, 13 aa, synthetic construct
  REGION 1..13: note = AtEIN2alt1
MGCKYRRRCSFRV

SEQ ID NO: 242 - protein, 13 aa, synthetic construct
  REGION 1..13: note = AlEIN2alt1
MGCKYRRRCSFRV

SEQ ID NO: 243 - protein, 17 aa, synthetic construct
  REGION 1..17: note = AtAP2alt1
MGISYLGLWETSLLRWI

SEQ ID NO: 244 - protein, 17 aa, synthetic construct
  REGION 1..17: note = AlAP2alt1
MGISYLGLWETSLLRWI

SEQ ID NO: 245 - protein, 17 aa, synthetic construct
  REGION 1..17: note = BoAP2alt1
MGVSYLGLWQTGLLRWI

SEQ ID NO: 246 - protein, 17 aa, synthetic construct
  REGION 1..17: note = CgAP2alt1
MGISYLGLWETGLLRWI

SEQ ID NO: 247 - protein, 17 aa, synthetic construct
  REGION 1..17: note = CrAP2alt1
MGISYLGLWETGLLRWI

SEQ ID NO: 248 - protein, 17 aa, synthetic construct
  REGION 1..17: note = BrAP2alt1
MGISYLGLWEAGLLRWI

SEQ ID NO: 249 - protein, 17 aa, synthetic construct
  REGION 1..17: note = BsAP2alt1
MGISYMGLWETGLPRWI

SEQ ID NO: 250 - protein, 27 aa, synthetic construct
  REGION 1..27: note = EsAP2alt1
MGISYLGLWETGLLRAYDRAAIKFPWS

SEQ ID NO: 251 - protein, 27 aa, synthetic construct
  REGION 1..27: note = ThAP2alt1
MGVSYLGLWKTSLFRWIRHSTCSCSRI

SEQ ID NO: 252 - protein, 30 aa, synthetic construct; identical to SEQ ID NO: 224
  REGION 1..30: note = altPEP_DCL1

SEQ ID NO: 253 - protein, 19 aa, synthetic construct
  REGION 1..19: note = altPEP_CYP78A8
MSLSGFLASKLTLQISRRF

SEQ ID NO: 254 - protein, 38 aa, synthetic construct
  REGION 1..38: note = altPEP_PRP39
MRSIQGFHTWSPGMLRKLGGLLSKHLTMYNRQNLSWKH

SEQ ID NO: 255 - protein, 46 aa, synthetic construct
  REGION 1..46: note = altPEP_PIF3
MTLKKRVLKARQMCFRPEFLELKTLKIRFLTSLLLLLRLKITKRLA

SEQ ID NO: 256 - protein, 70 aa, synthetic construct
  REGION 1..70: note = altPEP_PIF3
MQVIQTDSKVCLAEHLLLIYFPVVVQRDLMLLALTSAVSEFLLVKNVMRESEILTRLKHITSLTTLSLDS

SEQ ID NO: 257 - protein, 63 aa, synthetic construct
  REGION 1..63: note = altPEP_IQD28
MWFLQSMVMSPRIISSQGMTWEKSSLNKLQLRFKLPSELIRHVEHFELLKVSSGCRQLSVVTW

SEQ ID NO: 258 - protein, 16 aa, synthetic construct
  REGION 1..16: note = altPEP_AT59
MLLSLIATGRNDKVMH

SEQ ID NO: 259 - protein, 54 aa, synthetic construct
  REGION 1..54: note = altPEP_AT59
MDTEETPNGNSFIKSRISRNRLISSSPIWSSSENRIRRIEIGALRFRRGASSED

SEQ ID NO: 260 - protein, 15 aa, synthetic construct
  REGION 1..15: note = altPEP_ABCG11
MHMSSLHKLGFHVLR

SEQ ID NO: 261 - protein, 41 aa, synthetic construct
  REGION 1..41: note = altPEP_ABCG11
MRRGREKLLVTPSLKTLMISFSSLALSLSWNHQTPRGKKMS

SEQ ID NO: 262 - protein, 125 aa, synthetic construct
  REGION 1..125: note = altPEP_ABCG11
MFDLLLLFPWEIQVTQIRRMHGAWMHLKTKRTGRKLCMRMKLVADGVKRKEKLVYSVLGKLIDEKQSVVLTVSQAEKLVTLKMLLLLIGGMMLILVLLYMNPGATTNGHLGGVLMTKRKKLVVRR

SEQ ID NO: 263 - protein, 87 aa, synthetic construct
  REGION 1..87: note = altPEP_ABCG11
MVTQSPASSPSLSPDVLLPFPLPGESHRSLMYLLEKMLVMRYVSRIELRVKHGLSTSQMEFYFVRVCPIQCLMIILLSYWMRLTRGV

SEQ ID NO: 264 - protein, 13 aa, synthetic construct
  REGION 1..13: note = altPEP_ABCG11
MGFTLKPLSFSIR

SEQ ID NO: 265 - protein, 58 aa, synthetic construct
  REGION 1..58: note = altPEP_ABCG11
MLSSPRSTDEEKLPSLLLAATLTPMSLVLYWKKQVLSQSGNFMQRLFLENIRISQGEC

SEQ ID NO: 266 - protein, 52 aa, synthetic construct
  REGION 1..52: note = altPEP_ABCG11
MLVNISLVSFSWMKSMLLVDAVLVKELVLIVKFKEHLWSYLTSLMGLTNLER

SEQ ID NO: 267 - protein, 52 aa, synthetic construct
  REGION 1..52: note = altPEP_RD21
MLLSSLSRMVESIQTKIILTRVLMELVTRSGKTLKLSLSIHMRMFQLTARNR

SEQ ID NO: 268 - protein, 42 aa, synthetic construct
  REGION 1..42: note = altPEP_RD21
MPGLQVQEIDIHWNHEFKPNTSERVYGRHGLQLLREYVSLDC

SEQ ID NO: 269 - protein, 13 aa, synthetic construct
  REGION 1..13: note = altPEP_RD21
MVEEVVSRIPDFF

SEQ ID NO: 270 - protein, 33 aa, synthetic construct
  REGION 1..33: note = altPEP_RD21
MSSLENRKMDHSMDIRVEDFCRCLRLTISKTNI

SEQ ID NO: 271 - protein, 175 aa, synthetic construct
  REGION 1..175: note = altPEP_LRX2
MVKKIKDRLKSVLLQPHALLIVVSLGVTTFSLLHHRLLRCHPPFEYFLHRLRLPRCRLHLEQLRHRRLLRCHHLLEQLRHRLLLKCRLLLEQLRHHLLLRCRLPSRRILLLHHRQSMSHLRLLLLPKCHLPLGHTLHHHHYHHHHHLLLHHTSTHLLHHRHHLLLRHTFTHLLRP

SEQ ID NO: 272 - protein, 61 aa, synthetic construct
  REGION 1..61: note = altPEP_LRX2
MLFNSPPKSPKGSQTATLPSGTETCIAPDLTTRSRKRILISSRSTPRIYWMLELKSLCPRD

SEQ ID NO: 273 - protein, 58 aa, synthetic construct
  REGION 1..58: note = altPEP_LRX2
MSGKVWKEEMLILCGLGCIMNNKKKRLIIVIPITVLSLKAWQVKVINLPIMRIWVLLM

SEQ ID NO: 274 - protein, 123 aa, synthetic construct
  REGION 1..123: note = altPEP_JAC1
MTWTSILVTCLVVLLRDALKLLVTKSHDIASVNPLSGVVTSSSTSAICFRKTRSLYSAKIHHLSGVVSPLMISSTIFSELMNHHHFRVLGSLARLTNPNLLQGLRLHPNSVFLQKQQRFQHLI

SEQ ID NO: 275 - protein, 17 aa, synthetic construct
  REGION 1..17: note = altPEP_JAC1
MVIMVLIKKEGLFTLKD

SEQ ID NO: 276 - protein, 22 aa, synthetic construct
  REGION 1..22: note = altPEP_JAC1
MDSVMQKLRLESTIIARTKVAC

SEQ ID NO: 277 - protein, 87 aa, synthetic construct
  REGION 1..87: note = altPEP_PSKR1
MIKSSLMMTFWTQQIVLIKLTSLAVAGLVWFTKQRYQTVRKLRSRSYPVIAVKSKENSKQKLKHSQEHSIQILFFSEDSVSTKTTGF

SEQ ID NO: 278 - protein, 14 aa, synthetic construct
  REGION 1..14: note = altPEP_PSKR1
MLEEYLGRDKTNRS

SEQ ID NO: 279 - protein, 138 aa, synthetic construct
  REGION 1..138: note = altPEP_PSKR1
MKSLMGKKKKNPKITKVELGEAKEDKKAVVVVAKEDKKAVAVVAKEDKRVAVVAKEDKRVAVVAKEDKRAVAVVAKGDRRAVAVVDKGDRRVVVGAAKEDRRVAGAKEDRRVAGAKEDRRAAVAKEDRRAAVAAAKEE

SEQ ID NO: 280 - protein, 63 aa, synthetic construct
  REGION 1..63: note = altPEP_PSKR1
MKVLDVLIGKVLQRYHMRTSSLWTGHLNHMLSFIHLFLILETRSLTTENLYGVMKSEMVKWII

SEQ ID NO: 281 - protein, 64 aa, synthetic construct
  REGION 1..64: note = altPEP_PSKR1
MRNSIFSSSPGVLRISRCLKRSSMYQKDFQWEEDYGIFKCIQRETLEEIKNGYPFFCGYLVVKP

SEQ ID NO: 282 - protein, 92 aa, synthetic construct
  REGION 1..92: note = altPEP_PSKR1
MVTTYAQRDLKLIMLERILSLRGITLQLRKLLKLTSHSPLLVKMSWLLIQLGTYQKLTNFPSQKGMFTPFKKLAKLQMKVEGLLFSLESWLL

SEQ ID NO: 283 - protein, 131 aa, synthetic construct
  REGION 1..131: note = altPEP_FVE
MLNLLLPCAQRNPLCSPEARTSQLFCGVSKITSQRLGQIPNHLDLSSNRLVKVLIRMRVLLLAHEVYIMAMKIQLKMWHSARRVHKNSAVLVMILALYYGMRELAQTLSRRLKKRMMLIFIVLIGILMTTI

SEQ ID NO: 284 - protein, 99 aa, synthetic construct
  REGION 1..99: note = altPEP_FVE
MMEISQLKPILRLNTKTLVATIISPKHSNLRLNINPVDSLLSMEILLPQMDMDLVLLHLTMTFPRLLNLPLTMLTETAAAAVVMRSSLPMVRFFQIQMR

SEQ ID NO: 285 - protein, 37 aa, synthetic construct
  REGION 1..37: note = altPEP_UBA1A
MLHTGKSSFMVFHGRRLARLLLESLKVMVRLKSVPLL

SEQ ID NO: 286 - protein, 49 aa, synthetic construct
  REGION 1..49: note = altPEP_CIPK3
MKLEEQLEKERLLKLSLLETLKLENLLLSRFLIKRKFSSIKWLNRLEER

SEQ ID NO: 287 - protein, 80 aa, synthetic construct
  REGION 1..80: note = altPEP_CIPK3
MITTTKSKALMSMLVKIMLVTRRVMVVVMVIVTLKLEVTSVMRAMISLMMTIMVKMISKAIATSLEEVSLCMKMVNLTGL

SEQ ID NO: 288 - protein, 16 aa, synthetic construct
  REGION 1..16: note = altPEP_APG9
MTSCNSLRISQLILKV

SEQ ID NO: 289 - protein, 20 aa, synthetic construct
  REGION 1..20: note = altPEP_APG9
MLKTVQERKKKPFLDVGKKK

SEQ ID NO: 290 - protein, 23 aa, synthetic construct
  REGION 1..23: note = altPEP_COI1
MLLLGLLRFLTTLGSSNRCTSDG

SEQ ID NO: 291 - protein, 24 aa, synthetic construct
  REGION 1..24: note = altPEP_COI1
MVYSSLSSPRLRVLDILIRGWSHY

SEQ ID NO: 292 - protein, 25 aa, synthetic construct
  REGION 1..25: note = altPEP_COI1
MFRIRSLSIKITALVIKVVLNRVLE

SEQ ID NO: 293 - protein, 15 aa, synthetic construct
  REGION 1..15: note = altPEP_COI1
MIGEIIVRENLRTIK

SEQ ID NO: 294 - protein, 43 aa, synthetic construct
  REGION 1..43: note = altPEP_COI1
MMIGQCQLKRSKLSLLSPLKQQPTLRLKLFLKFSTACGMQIVS

SEQ ID NO: 295 - protein, 212 aa, synthetic construct
  REGION 1..212: note = altPEP_COI1
MLSALTLCRPPADPLNTSLSWQPFIAAGSTGSLSSRCRSTIPSLKSPSSYNNIWLKAFIIISTTTTTMILPPATSLMEDPGKKKPSPSVVSTRTNVNHEEPHSSSSPSVSRPRNMLEDIRPMLSKALNSTKMSSLDLNSPSFWILLISSERLVGITNFQGRPSFCSTRTPREPKESFFSLEFLFSATPRMNTLWNLSQPLSKVLSTIFFNGV

SEQ ID NO: 296 - protein, 44 aa, synthetic construct
  REGION 1..44: note = altPEP_COI1
MMNRKSLLSTKHRQNRRWPVMKMTWQERGCRPRMSSIEQEIKNL

SEQ ID NO: 297 - protein, 42 aa, synthetic construct
  REGION 1..42: note = altPEP_COI1
MGMHSGERSYLEFVNFVHNIWNVVSGSKLGSLSLPRKLCKRC

SEQ ID NO: 298 - protein, 18 aa, synthetic construct
  REGION 1..18: note = altPEP_COI1
MRNRIEETGLRSLDLPMV

SEQ ID NO: 299 - protein, 19 aa, synthetic construct
  REGION 1..19: note = altPEP_MFP2
MRNSWRLELAELKLISRAV

SEQ ID NO: 300 - protein, 73 aa, synthetic construct
  REGION 1..73: note = altPEP_MFP2
MALVIQRKTQSSTIGGSICRHSRSPITLLHVSLSMLSKVFVKRHLLYFKSLVYVTLLALMDGIWLPIQIYHHL

SEQ ID NO: 301 - protein, 34 aa, synthetic construct
  REGION 1..34: note = altPEP_MFP2
MSGRLDQKSLLHFSLVFRIGGLSLRGLIRTEAVE

SEQ ID NO: 302 - protein, 37 aa, synthetic construct
  REGION 1..37: note = altPEP_COBL8
MSFSSQPKELIQVLSIRRIASLVLKQNTIKILTSLMS

SEQ ID NO: 303 - protein, 31 aa, synthetic construct
  REGION 1..31: note = altPEP_IAA31
MTLLSLLRFIWKVFPLEENSTFAYSQATRVY

SEQ ID NO: 304 - protein, 56 aa, synthetic construct
  REGION 1..56: note = altPEP_CYP705A18
MCCWQLIETKTQSIRSLGIISSRFLWTFSLGALIPRSKQHNGLWLRSLITLTFFRH

SEQ ID NO: 305 - protein, 10 aa, synthetic construct
  REGION 1..10: note = altPEP_AAE16
MLTSFLRPSV

SEQ ID NO: 306 - protein, 36 aa, synthetic construct
  REGION 1..36: note = altPEP_AAE16
MFRHSICLREVLSLLRKMRRGKLGLTLRSRKKRGKL

SEQ ID NO: 307 - protein, 39 aa, synthetic construct
  REGION 1..39: note = altPEP_CYP71B26
MSDGSSISSMVYKGGERKAFEILMRSMNKFLICIRKRRK

SEQ ID NO: 308 - protein, 31 aa, synthetic construct
  REGION 1..31: note = altPEP_CYP71B26
MNVSLLREVMDLVLEQSDLMQKPVALQMMGK

SEQ ID NO: 309 - protein, 31 aa, synthetic construct; identical to SEQ ID NO: 308
  REGION 1..31: note = altPEP_CYP71B26

SEQ ID NO: 310 - protein, 47 aa, synthetic construct
  REGION 1..47: note = altPEP_CYP71B26
MRTKRCRFLGILPLQQKEAENLAEKMLLSRRRLCSTNFFLRWKDKKR

SEQ ID NO: 311 - protein, 22 aa, synthetic construct
  REGION 1..22: note = altPEP_CYP71B26
MALFSILRKVYGRGTSYWIEIG

SEQ ID NO: 312 - protein, 37 aa, synthetic construct
  REGION 1..37: note = altPEP_CYP71B26
MATNVIPVSLLYSTRHAPTIRISPTLLKRVASFFIIQ

SEQ ID NO: 313 - protein, 76 aa, synthetic construct
  REGION 1..76: note = altPEP_KP1
MRFKISKAPLEFTAESDLSSKNKKICNQLWIILEKMVILSLTILSSRRKMHGKYFPSTKYLDRLSHKSKSTLTLNR

SEQ ID NO: 314 - protein, 19 aa, synthetic construct
  REGION 1..19: note = altPEP_KP1
MILLIGWISRLLALLSRDF

SEQ ID NO: 315 - protein, 40 aa, synthetic construct
  REGION 1..40: note = altPEP_PEN3
MGKLLFSQVRFDMSSTTNVFKTNLTGKFHPSEDACSEDFA

SEQ ID NO: 316 - protein, 53 aa, synthetic construct
  REGION 1..53: note = altPEP_PEN3
MLNLVTLKNIIFRLCCLLRILKDVVTLHSHVLIGPVVLVVILTRTMPLRSRRS

SEQ ID NO: 317 - protein, 38 aa, synthetic construct
  REGION 1..38: note = altPEP_HEXO1
MKLIQIAGKTQLMLKNGFRVATSRLRTHINTLYSERNR

SEQ ID NO: 318 - protein, 54 aa, synthetic construct
  REGION 1..54: note = altPEP_GDI2
MILTQCRSSYPRNSWPANRICMSSVVRTPTTLLPRENSLHLCLQMQRLITLKPN

SEQ ID NO: 319 - protein, 67 aa, synthetic construct
  REGION 1..67: note = altPEP_GDI2
MKILTLLSLMKILSLKIQSLLSLIKSTVLYVLCSSVGSRIFICGYMANKDIHCLLKFSLVRQLLNSH

SEQ ID NO: 320 - protein, 86 aa, synthetic construct
  REGION 1..86: note = altPEP_GDI2
MAILILRVSVTTSVLALLALLRSLALRLVPAVVLRLVISSVHRRVRCISSFALLLLHRRILKSLTRLHAATLSLTSRRGCLQAVVL

SEQ ID NO: 321 - protein, 29 aa, synthetic construct
  REGION 1..29: note = altPEP_GDI2
MILFLLSKRKSVKSMKRVSQVLETSLSPL

SEQ ID NO: 322 - protein, 28 aa, synthetic construct
  REGION 1..28: note = altPEP_GDI2
MLLIPLSRLRVHYRCLSLVICIALIRSH

SEQ ID NO: 323 - protein, 28 aa, synthetic construct
  REGION 1..28: note = altPEP_SFR6
MLLLVLPRPVPGFQAPRQIHLFGYQYIS

SEQ ID NO: 324 - protein, 96 aa, synthetic construct
  REGION 1..96: note = altPEP_CRK34
MSQVTTAFSTPSLVKLPIESSSMACASLGLNQRLVQIVLKVHQTRYQRVVLTKQMLIHGLIVVWYVIPTFLSLVHSLWNLLKLSIIREISKIQALI

SEQ ID NO: 325 - protein, 10 aa, synthetic construct
  REGION 1..10: note = altPEP_AAE15
MLTSFLRPSV

SEQ ID NO: 326 - protein, 42 aa, synthetic construct
  REGION 1..42: note = altPEP_CYP97B3
MEEFINLRLVQKPLLSSQIPLLQGMSSGKMLFLMTREFLLRS

SEQ ID NO: 327 - protein, 11 aa, synthetic construct
  REGION 1..11: note = altPEP_CYP97B3
MLSSLELKWLP

SEQ ID NO: 328 - protein, 39 aa, synthetic construct
  REGION 1..39: note = altPEP_CYP97B3
MTVGYVVVQQIVSGRRRRATSVLPRSIGAAVGFVDQRIG

SEQ ID NO: 329 - protein, 19 aa, synthetic construct
  REGION 1..19: note = altPEP_CYP97B3
MKLLEQNQRGLVWILLEHT

SEQ ID NO: 330 - protein, 11 aa, synthetic construct
  REGION 1..11: note = altPEP_AMB2726
MFRVLSLLDKR

SEQ ID NO: 331 - protein, 51 aa, synthetic construct
  REGION 1..51: note = altPEP_HSL1
MHLHCVRGSEETIQDAHAAEAEQRRGRTGKPAGAVTVRVQGRDRSGEHSSG

SEQ ID NO: 332 - protein, 67 aa, synthetic construct
  REGION 1..67: note = altPEP_HSL1
MSSSGCYLHSPARTHLRYLAGVWFPLSFRLLSLLSKCPRLFFFFSGGSDSTADSQRRRPSSSTIFSG

SEQ ID NO: 333 - protein, 103 aa, synthetic construct
  REGION 1..103: note = altPEP_HSL1
MMMWTEMVLVLQEDLLDRLIGILLERGMVKMLGIRVLRLVRLLEGILKMFWQGWTYKQLVLRLRLNRCLMGWMTLMVQKTFHLMSWLECKVQCFIWLRMPLLF

SEQ ID NO: 334 - protein, 46 aa, synthetic construct
  REGION 1..46: note = altPEP_ANAC076
MRKEMNGISSATKIRNIQQEQEQTERPWLGFGKPRAETRLFTTSQN

SEQ ID NO: 335 - protein, 15 aa, synthetic construct
  REGION 1..15: note = altPEP_STN8
MILIGGGKGLGRGQT

SEQ ID NO: 336 - protein, 15 aa, synthetic construct
  REGION 1..15: note = altPEP_EMB175
MEFSRAILGASRFGL

SEQ ID NO: 337 - protein, 27 aa, synthetic construct
  REGION 1..27: note = altPEP_EMB175
MESLMSLRLGSTMGYHTGLWTLICTSG

SEQ ID NO: 338 - protein, 20 aa, synthetic construct
  REGION 1..20: note = altPEP_EMB175
MMILRIGRRNLLSVYAKPGR

SEQ ID NO: 339 - protein, 32 aa, synthetic construct
  REGION 1..32: note = altPEP_EMB175
MLSLEGLRVTGSLESRSNCFTKCENQRLTRTR

SEQ ID NO: 340 - protein, 34 aa, synthetic construct
  REGION 1..34: note = altPEP_EMB175
MLMLPPNKPNEPRPCSRESLRVIVSLLVPSHTTR

SEQ ID NO: 341 - protein, 31 aa, synthetic construct
  REGION 1..31: note = altPEP_GCN2
MSTKARSSQSHTIDTSLNRLLTELLKSEMVV

SEQ ID NO: 342 - protein, 18 aa, synthetic construct
  REGION 1..18: note = altPEP_SPS1
MSLRIGLLNTKRKSFTSC

SEQ ID NO: 343 - protein, 26 aa, synthetic construct
  REGION 1..26: note = altPEP_SPS1
MLIPSILKVSSTVKLISKPSRRTYPL

SEQ ID NO: 344 - protein, 28 aa, synthetic construct
  REGION 1..28: note = altPEP_SPS1
MMSAKLSTEEQRMAIVNRTLWRRKMMRI

SEQ ID NO: 345 - protein, 107 aa, synthetic construct
  REGION 1..107: note = altPEP_SPS1
MTLKIVASGILLGMLPWLPLNPLLPEIEICFPIHPRFLIRIMFRPQIQILRLKSRKQAVRVQTSKMTAEKRLLEILQWKGRTSALVMTFSSCWSLVQCVLWRLCIKE

SEQ ID NO: 346 - protein, 19 aa, synthetic construct
  REGION 1..19: note = altPEP_BZO2H3
MVEPMNQNKPLLLLPKLHQ

SEQ ID NO: 347 - protein, 33 aa, synthetic construct
  REGION 1..33: note = altPEP_VIM3
MLPNVLLRQLQPRFIILSATKTVLRKHLLPSGQ

SEQ ID NO: 348 - protein, 31 aa, synthetic construct
  REGION 1..31: note = altPEP_EMB9
MENLSLLPLLMIFSIQSNRFLNSLFKRKMVL

SEQ ID NO: 349 - protein, 26 aa, synthetic construct
  REGION 1..26: note = altPEP_EMB9
MASEIGAKANVSDFLVPKIQVSWNFV

SEQ ID NO: 350 - protein, 52 aa, synthetic construct
  REGION 1..52: note = altPEP_RPT4A
MPVNISLVSYSWMKSMLLVGVVLVRELVLIVKFKERLWSYSTSLMGLTTSER

SEQ ID NO: 351 - protein, 23 aa, synthetic construct
  REGION 1..23: note = altPEP_TOPP6
MTRNQSSVSEAEVVLKLASLILK

SEQ ID NO: 352 - protein, 13 aa, synthetic construct
  REGION 1..13: note = altPEP_DUR3
MSSFRFLDQILRW

SEQ ID NO: 353 - protein, 28 aa, synthetic construct
  REGION 1..28: note = altPEP_DUR3
MEEPIWKTQLFFCSSRSETKTKAETRTN

SEQ ID NO: 354 - protein, 11 aa, synthetic construct
  REGION 1..11: note = altPEP_DUR3
MTFIGLEKLWK

SEQ ID NO: 355 - protein, 14 aa, synthetic construct
  REGION 1..14: note = altPEP_ARLB1
MQLAQPALKILNLL

SEQ ID NO: 356 - protein, 12 aa, synthetic construct
  REGION 1..12: note = altPEP_ARLB1
MIWVADRPLVQS

SEQ ID NO: 357 - protein, 15 aa, synthetic construct
  REGION 1..15: note = altPEP_HDA18
MDLLPFLRSSMKTWT

SEQ ID NO: 358 - protein, 36 aa, synthetic construct
  REGION 1..36: note = altPEP_HDA18
MSVEERAVALEVRVVVADKAVAEEKAAAEEQEQVIW

SEQ ID NO: 359 - protein, 58 aa, synthetic construct
  REGION 1..58: note = altPEP_HDA18
MVVRRVRKVALISVRHMVVGNVAHGEETGNARNSLEERAVYALRITVCHKIKLEARLV

SEQ ID NO: 360 - protein, 58 aa, synthetic construct
  REGION 1..58: note = altPEP_CESA6
MENRLWHVTNVHSLCVDLAMSTKDEKAIKLVHSAKPVSNVLKEVQELKVMKRKMTLMI

SEQ ID NO: 361 - protein, 31 aa, synthetic construct
  REGION 1..31: note = altPEP_FNR1
MKLRSLDLLARKCLCQKTPMPPSSCLEQELE

SEQ ID NO: 362 - protein, 10 aa, synthetic construct
  REGION 1..10: note = altPEP_FNR1
MKLAPGLIRF

SEQ ID NO: 363 - protein, 11 aa, synthetic construct
  REGION 1..11: note = altPEP_FNR1
MKSLESLLRIG

SEQ ID NO: 364 - protein, 13 aa, synthetic construct
  REGION 1..13: note = EIN2alt1
MGCKYRRRCSFRV

SEQ ID NO: 365 - protein, 23 aa, synthetic construct
  REGION 1..23: note = EIN2alt2
MTWWQLLCFSILPPSYANMLQLA

SEQ ID NO: 366 - protein, 13 aa, synthetic construct
  REGION 1..13: note = EIN2alt3
MPICCSSHKRCDW

SEQ ID NO: 367 - DNA, 20 bp, synthetic construct
  source 1..20: mol_type = other DNA; note = LUC q5
gccgttgttgttttggagca

SEQ ID NO: 368 - DNA, 20 bp, synthetic construct
  source 1..20: mol_type = other DNA; note = LUC q3
tgcgtcgagttttccggtaa

SEQ ID NO: 369 - DNA, 19 bp, synthetic construct
  source 1..19: mol_type = other DNA; note = Mtubi q5
gcagatagacacgctggga

SEQ ID NO: 370 - DNA, 20 bp, synthetic construct
  source 1..20: mol_type = other DNA; note = Mtubi q3
aactcttgggcaggcaataa

SEQ ID NO: 371 - DNA, 20 bp, synthetic construct
  source 1..20: mol_type = other DNA; note = MtNsp1 q5
attcaaccagttcggcattc

SEQ ID NO: 372 - DNA, 20 bp, synthetic construct
  source 1..20: mol_type = other DNA; note = MtNsp1 q3
ctgcaaaccctgcttctttc

SEQ ID NO: 373 - DNA, 20 bp, synthetic construct
  source 1..20: mol_type = other DNA; note = At-Actin q5
ggtaacattgtgctcagtgg

SEQ ID NO: 374 - DNA, 20 bp, synthetic construct
  source 1..20: mol_type = other DNA; note = At-Actin q3
ctcggccttggagatccaca

SEQ ID NO: 375 - RNA, 15 nt, synthetic construct
  source 1..15: mol_type = other RNA
atgccgacaagattc

SEQ ID NO: 376 - RNA, 15 nt, synthetic construct
  source 1..15: mol_type = other RNA
ccggctacggtataa

SEQ ID NO: 377 - RNA, 12 nt, synthetic construct
  source 1..12: mol_type = other RNA
ccgacaagattc

SEQ ID NO: 378 - RNA, 12 nt, synthetic construct
  source 1..12: mol_type = other RNA
tgccgacaagat

SEQ ID NO: 379 - RNA, 12 nt, synthetic construct
  source 1..12: mol_type = other RNA
gccgacaagatt

SEQ ID NO: 380 - protein, 11 aa, synthetic construct
  source 1..11: note = Peptide TAT
YGRKKRRQRRR

SEQ ID NO: 381 - DNA, 807 bp, Arabidopsis thaliana
  source 1..807: mol_type = other DNA; note = ORF of protein Magnesium dechelatase
atgtgtagtttgtcggcgattatgttgttaccaacgaagctgaaaccagcttattcagacaaacggagtaacagtagcagcagcagctcactcttcttcaacaatagaagatccaagaagaagaaccaatcgattgttcccgttgcaaggttgtttggaccggcgattttcgaatcatccaaattgaaagtactcttcttaggggttgatgagaagaagcatccttcaacgctccctaggacttacacactcactcacagtgacattacagctaaactaaccttagctatttctcaatccataaacaactctcagttgcaaggatgggcaaataggctataccgggatgaagttgtggcagaatggaagaaagtgaaagggaaaatgtcgcttcacgttcattgtcacataagcggtggccatttccttttagatctctttgcaaagtttcgatatttcatcttttgcaaagaactacctgtggtgttgaaggcttttgtgcatggagatgggaacttgttgaacaactatcctgagctacaagaagctcttgtttgggtctatttccattctaatgtcaatgagttcaacaaagtcgagtgttggggtccgctttgggaagctgtttcgcctgatggtcacaagactgagactcttcccgaggctcggtgtgcggacgagtgtagttgttgttttccaaccgttagctcgattccatggtctcatagtcttagtaatgaaggtgtaaatggttactctgggactcagactgagggaattgctactccaaatccggagaaactctag

SEQ ID NO: 382 - DNA, 1329 bp, Arabidopsis thaliana
  source 1..1329: mol_type = other DNA; note = ORF of protein ABA insensitive 5
atggtaactagagaaacgaagttgacgtcagagcgagaagtagagtcgtccatggcgcaagcgagacataatggaggaggtggtggtgagaatcatccgtttacttctttgggaagacaatcctctatctactcattgacccttgacgagttccaacatgctttatgtgagaacggcaagaactttgggtccatgaacatggacgagtttcttgtctctatttggaacgcagaggagaataataacaatcaacaacaagcagcagcagctgcaggttcacattctgttccggctaatcacaatggtttcaacaacaacaataacaatggaggcgagggtggtgttggtgtctttagtggtggttctagaggcaacgaagatgctaacaataagagagggatagcgaacgagtctagtcttcctcgacaaggctctttgacacttccagctccgctttgtaggaagactgttgatgaggtttggtctgagatacatagaggtggtggtagcggtaatggaggagacagcaatggacgtagtagtagtagtaatggacagaacaatgctcagaacggcggtgagactgcggctagacaaccgacttttggagagatgacacttgaggatttcttggtgaaggctggtgtggttagagaacatcccactaatcctaaacctaatccaaacccgaaccaaaaccaaaacccgtctagtgtaatacccgcagctgcacagcaacagctttatggtgtgtttcaaggaaccggtgatccttcattcccgggtcaagctatgggtgtgggtgacccatcaggttatgctaaaaggacaggaggaggagggtatcagcaggcgccaccagttcaggcaggtgtttgctatggaggtggcgttgggtttggagcgggtggacagcaaatgggaatggttggaccgttaagcccggtgtcttcagatggattaggacatggacaagtggataacataggaggtcagtatggagtagatatgggagggctaaggggaaggaaaagagtagtggatggtccagtggagaaagtagtggagagaagacagaggaggatgatcaagaaccgcgagtctgctgctagatctagagcaagaaaacaagcatatacagtggaattggaagctgaacttaaccagttgaaagaagagaatgcgcagctaaaacatgcattggcggagttggagaggaagaggaagcaacagtattttgagagtttgaagtcaagggcacaaccgaaattgccgaaatcgaacgggagattgcggacattgatgaggaacccgagttgtccactctaa

SEQ ID NO: 383 - DNA, 3284 bp, Arabidopsis thaliana
  source 1..3284: mol_type = other DNA; note = ORF of protein Heat shock protein 101
caaaaagagtggaattgacatttgtttctctcacactctctctcgaattctctggtagcttctagttctatgcaaaaaaacgacgatagttctctatctttccagatgaatctccttccatatacaaaagcagtcatgcctcctcgctctctcgcaattcacaaagtatccaaacatctaaagttatcaattttacaacattaccgctataatctgcttgattctctgcaaaaagagaagactttttaccgagaagaagtcctctggctcattgaagaaactcaacgaaacaaacccagttctcatatatcgttttaagggattacaaaagctaatcgaagatgaatccagagaaattcacacacaagacaaacgagacaattgctacagctcatgagctagctgtgaatgcaggacatgctcaattcactcctttgcatttagctggtgctttgatctctgatcccaccggtatatttcctcaagcaatctctagtgccggtggcgagaacgcagctcaatctgctgaaagagtgatcaatcaagccttgaagaagcttccttcacaatctcctccacctgatgatattccagcgagttctagtcttattaaggtcattcgtcgtgctcaagctgctcagaagtcacgaggtgatactcatttggctgttgaccagttgattatgggtcttcttgaagattctcaaatcagggatttgttgaacgaagtcggtgtagcgacggcgagggtaaagtctgaggttgagaagcttcgtgggaaagaagggaagaaagttgagagtgcttcaggggacacaaattttcaagctttaaagacttatggaagagatttggttgagcaagcagggaagcttgatcctgtgattggtcgtgatgaggagattagaagagtcgtgaggattctttcgaggagaacgaagaacaatcctgtgcttattggagagccaggagttggtaaaacagctgtggttgaaggtttagcacaaaggattgtgaaaggagatgtgcccaacagtcttactgatgtgagattaatttcgttggacatgggtgcgttagttgctggtgctaaataccgaggagagtttgaagaaaggttgaaatctgttttgaaagaagttgaggacgctgaaggcaaagtgattctctttattgatgagattcatttggttcttggtgctggcaaaactgaagggtcgatggatgcagctaatctgttcaagcccatgttagctagagggcagcttcgatgcattggtgctacaacgcttgaagaatacaggaaatatgttgagaaagatgctgcctttgagaggaggttccaacaagtctatgttgcggagccaagtgtgcctgacaccattagtatccttagaggactcaaggagaagtatgagggacatcatggtgtgcgaatccaagacagagctcttataaatgctgctcagctgtctgctcgttacataactggtcggcatttaccggataaagcaattgatttggttgatgaggcttgtgcgaatgtgagagtccagcttgatagtcaacctgaagagattgataaccttgaaaggaagaggatgcagctggaaattgaacttcacgccttggaaagggagaaggataaagccagcaaagctcgacttatagaggtgcggaaagagcttgatgacctgagagacaagcttcagcctctcacgatgaaatacagaaaggagaaagagagaattgatgagattcgaaggcttaaacagaaaagagaagagctcatgttttctttgcaggaggcagaacgaagatatgaccttgcaagagctgctgatctaagatatggcgcaattcaagaagtggaatctgcaattgcccaacttgaaggaacttcttctgaagagaatgtgatgctcacagaaaacgttgggcctgaacacattgctgaggttgtgagccgttggacagggattccagtgacgagacttggccaaaatgagaaggagaggttgattggtcttgctgataggttgcataagcgggttgtgggacagaatcaagcggtaaatgcagtttctgaggcaattctaaggtcaagggcaggacttggaaggccacaacagccaactggatcattcttattccttggaccaactggtgttggcaaaactgagctcgccaaggctcttgctgagcagctgtttgatgatgaaaacctcttagttcggattgatatgtcggaatatatggaacaacactctgtctctcgcctcattggggcaccaccagggtatgttggtcacgaggaaggtggacaactaactgaggctgtgaggaggcgaccttattgtgtcatactctttgatgaagtggagaaggctcatgttgctgtcttcaacactctgctccaagttttggatgatggtcgattgacagacgggcaaggcaggacagtcgatttcaggaactcggtgataatcatgacatcaaaccttggtgctgaacacctccttgcagggctaactgggaaagtaacaatggaagtggcccgggactgtgtgatgcgggaggtgaggaaacacttcagaccagagctcttgaacaggcttgacgagattgtggtgttcgaccccctttcacatgaccagttgaggaaagtagctcggcttcaaatgaaagacgttgctgtccggcttgctgaaagaggagttgctttggcagtcactgatgctgctttggactatatcttggcagagagttatgacccggtgtatggtgctaggcctataaggagatggatggagaagaaggtggtgacagaactgtcaaagatggttgtgcgtgaggaaatcgatgaaaactccactgtttacatagatgcaggcgctggtgatcttgtgtaccgggtagaaagtggaggtctagtggacgcttcaacaggcaagaagtcagatgtgctgattcatattgctaacgggccaaagagaagtgatgcagctcaggcggtgaagaagatgaggatcgaggaaatagaagatgacgataatgaggaaatgatcgaggattaaagtcttgacctccaacgtttgagttcttttgaggtgttgtgattgtatgacttgagctttcatgctctgttttggttttgactcttttggtagactataatggtgtggaactgtaaaaaagtctactttattatgttttcgtatatgtaaatcattttacataagaaatccattgtacgtcccataatcttttggggttttttcata

SEQ ID NO: 384 - DNA, 1254 bp, Medicago truncatula
  source 1..1254: mol_type = other DNA; note = ORF of protein ATP-dependent RNA helicase
atgaaatcatttacggatttgggcttatctgttcaattggtggaagcttgtgaaaaaatgcgttggtatagtccacttaagattcagacagaagtgattccccttgctctacaaggaaaagatgatgtgatcggaatttcaccaccgcgttctggtaaggccggggcttttgttcttcctatattgcaggccctcttagaagccggacccaacctaaacacgtcctttgcttgtgtactgtctccctcaagggaccttgtttttcgtattgctgagtattttcaagttctaggttctcaatttggtgtcaagtgtgctacccttgttgaagcgaacgacatcatagatcaaaccaaccagattttacagcaacctcatcttattgttggaacccttcgacaagtcttttatcacttaagactaacccaaggattttctcttgctagattaaaatacctggtcatacatgaggcagatctgctgttgaatgaccagttcgaggaacaacaactaaatgatattttatcaataattcctagtgagcggagaacttttcttttttcatctacaatgacagagaaggtccatatgatccaaagattgtctttaaggaaccctctaaagattgatgtatcttccaaatattctactgtggtcacacagctgcagcagagttgctttatgccagcaatgcttaaggattgctaccttgtatacattctcactgaaatgactggaagaaaatcaacggtattcactcagacttgtggttcagcgtttcttctggctttaattcttaagaatcttgatttcagagccatcccaattattagttatatgagccaggcaaagaaactaggagccctgaatgcgttcaagtctggaaagttcaatattctcctttgtagtgaagcagctaggaggggactagatattccagcggtagatatggtgattaactacaacatacctagagatcccaatgattatatgcatcgtgtgggatggactggtcatgttaatgctgccatttcttttgtgaacccatatgaagcgggacaacttgaaatgatagagagacatacaggcaagaagctaccggtgtatccggcaccatgtgagaatgttttgctttggagagggactgttagggaggccgaaagattagcacgaaaggaaatcaaagaatctggttggaagagtggaggatatttaggagacgaggaggtcaaaaagatctcaagtctgcgttga

SEQ ID NO: 385 - DNA, 909 bp, Arabidopsis thaliana
  source 1..909: mol_type = other DNA; note = ORF of protein WUSCHEL
ctttgtgaacaaaagtcgaatcaaacacacatggagccgccacagcatcagcatcatcatcatcaagccgaccaagaaagcggcaacaacaacaacaacaagtccggctctggtggttacacgtgtcgccagaccagcacgaggtggacaccgacgacggagcaaatcaaaatcctcaaagaactttactacaacaatgcaatccggtcaccaacagccgatcagatccagaagatcactgcaaggctgagacagttcggaaagattgagggcaagaacgtcttttactggttccagaaccataaggctcgtgagcgtcagaagaagagattcaacggaacaaacatgaccacaccatcttcatcacccaactcggttatgatggcggctaacgatcattatcatcctctacttcaccatcatcacggtgttcccatgcagagacctgctaattccgtcaacgttaaacttaaccaagaccatcatctctatcatcataacaagccatatcccagcttcaataacgggaatttaaatcatgcaagctcaggtactgaatgtggtgttgttaatgcttctaatggctacatgagtagccatgtctatggatctatggaacaagactgttctatgaattacaacaacgtaggtggaggatgggcaaacatggatcatcattactcatctgcaccttacaacttcttcgatagagcaaagcctctgtttggtctagaaggtcatcaagaagaagaagaatgtggtggcgatgcttatctggaacatcgacgtacgcttcctctcttccctatgcacggtgaagatcacatcaacggtggtagtggtgccatctggaagtatggccaatcggaagttcgcccttgcgcttctcttgagctacgtctgaactag

SEQ ID NO: 386 - protein, 19 aa, synthetic construct
  source 1..19: note = Scrambled cPEPluc-HA
RNYKNGRTHLYPYDVPDYA

SEQ ID NO: 387 - protein, 19 aa, synthetic construct
  source 1..19: note = cPEPluc-HA
KRYGLNTNHRYPYDVPDYA

SEQ ID NO: 388 - protein, 10 aa, synthetic construct
  source 1..10: note = cPEPluc 2 ORF1
DKYDLSNLHE

SEQ ID NO: 389 - protein, 10 aa, synthetic construct
  source 1..10: note = cPEPluc 3 ORF1
EHGKTMTEKE

SEQ ID NO: 390 - protein, 10 aa, synthetic construct
  source 1..10: note = cPEPluc 1 ORF2
RKARRHSIRW

SEQ ID NO: 391 - protein, 10 aa, synthetic construct
  source 1..10: note = cPEPluc 2 ORF2
RKSGKRLPRG

SEQ ID NO: 392 - protein, 9 aa, synthetic construct
  source 1..9: note = cPEPluc 3 ORF2
RPRRAERSP

SEQ ID NO: 393 - protein, 10 aa, synthetic construct
  source 1..10: note = cPEPluc 4 ORF2
NTILCQSPSI

SEQ ID NO: 394 - protein, 9 aa, synthetic construct
  source 1..9: note = cPEPluc 1 ORF3
KHKERPGAI

SEQ ID NO: 395 - protein, 10 aa, synthetic construct
  source 1..10: note = cPEPluc 2 ORF3
EVPSARYQAR

SEQ ID NO: 396 - protein, 10 aa, synthetic construct
  source 1..10: note = cPEPluc 3 ORF3
RKTRRKKNQR

SEQ ID NO: 397 - protein, 10 aa, synthetic construct
  source 1..10: note = cPEPluc 4 ORF3
IAQQYEHFAA

SEQ ID NO: 398 - protein, 5 aa, synthetic construct
  source 1..5: note = cPEPluc 5
NTNHR

SEQ ID NO: 399 - protein, 20 aa, synthetic construct
  source 1..20: note = cPEPluc 20
EMSVRLAEAMKRYGLNTNHR

SEQ ID NO: 400 - protein, 30 aa, synthetic construct
  source 1..30: note = cPEPluc 30
EVDITYAEYFEMSVRLAEAMKRYGLNTNHR

SEQ ID NO: 401 - protein, 40 aa, synthetic construct
  source 1..40: note = cPEPluc 40
GTIAFTDAHIEVDITYAEYFEMSVRLAEAMKRYGLNTNHR

SEQ ID NO: 402 - protein, 50 aa, synthetic construct
  source 1..50: note = cPEPluc 50
KAMKRYALVPGTIAFTDAHIEVDITYAEYFEMSVRLAEAMKRYGLNTNHR

SEQ ID NO: 403 - protein, 60 aa, synthetic construct
  source 1..60: note = cPEPluc 60
EDGTAGEQLHKAMKRYALVPGTIAFTDAHIEVDITYAEYFEMSVRLAEAMKRYGLNTNHR

SEQ ID NO: 404 - protein, 11 aa, synthetic construct
  source 1..11: note = cPEPhsp101
MQDMLNSLLCI

SEQ ID NO: 405 - protein, 11 aa, synthetic construct
  source 1..11: note = Scrambled cPEPhsp101
IDLMQNLMSCL

SEQ ID NO: 406 - protein, 10 aa, synthetic construct
  source 1..10: note = cPEPabi5
EKVVERRQRR

SEQ ID NO: 407 - protein, 10 aa, synthetic construct
  source 1..10: note = cPEPsgr1
EWKKVKGKMS

SEQ ID NO: 408 - protein, 10 aa, synthetic construct
  source 1..10: note = cPEPhsp101
EEYRKYVEKD

SEQ ID NO: 409 - protein, 9 aa, synthetic construct
  source 1..9: note = cPEPmrb1
TDGESETDE

SEQ ID NO: 410 - protein, 10 aa, synthetic construct
  source 1..10: note = cPEPshy2
DLKVYGGYTQ

SEQ ID NO: 411 - protein, 10 aa, synthetic construct
  source 1..10: note = cPEPskl
TETVGHEDAS

SEQ ID NO: 412 - protein, 10 aa, synthetic construct
  source 1..10: note = cPEPrh10
QRLSLRNPLK

SEQ ID NO: 413 - protein, 10 aa, synthetic construct
  source 1..10: note = cPEpjar1
AFRTFEQVWE

SEQ ID NO: 414 - protein, 10 aa, synthetic construct
  source 1..10: note = cPEPbak1
KRLKEERTQG

SEQ ID NO: 415 - protein, 10 aa, synthetic construct
  source 1..10: note = cPEPbri1
HQRSHGRRPA

SEQ ID NO: 416 - protein, 10 aa, synthetic construct
  source 1..10: note = cPEPwus
RWTPTTEQIK

SEQ ID NO: 417 - protein, 10 aa, synthetic construct
  source 1..10: note = cPEPein2
NPVERSRHRD

SEQ ID NO: 418 - protein, 11 aa, synthetic construct
  source 1..11: note = artificial cPEP/ irrelevant peptide
RTFNSKVSPKN